(19) United States
(12) Patent Application Publication
(10) Pub. No.: US 2026/0315604 A1
(43) Pub. Date: Oct. 15, 2026

(54) OTUB1 Small-Molecule Binders and OTUB1-recruiting Deubiquitinase-targeting Chimeras (DUBTACs)

(71) Applicants: ICAHN SCHOOL OF MEDICINE AT MOUNT SINAI, New York, NY (US); BETH ISRAEL DEACONESS MEDICAL CENTER, INC., Boston, MA (US)

(72) Inventors: Jian Jin, New York, NY (US); Wenyi Wei, Boston, MA (US); Yan Xiong, New York, NY (US); Xiangyang Song, New York, NY (US); Qiong Wu, New York, NY (US); Chao Qian, New York, NY (US); Zhijie Deng, New York, NY (US); Jing Liu, Boston, MA (US); Xufen Yu, New York, NY (US)

(21) Appl. No.: 19/476,898

(22) PCT Filed: Apr. 19, 2024
(86) PCT No.: PCT/US2024/025509

§ 371 Date: Oct. 20, 2025

Related U.S. Application Data
(60) Provisional application No. 63/497,651, filed on Apr. 21, 2023.

(51) Int. Cl. A61K 47/55; A61K 47/54

(52) U.S. Cl. CPC A61K 47/55; A61K 47/545; A61K 47/549

(57) ABSTRACT

Disclosed are compounds that are irreversible small-molecule binders of OTUB1 (also known as Otubain 1), and bivalent compounds (e.g. bi-functional small molecule compounds) which recruit OTUB1 through the irreversible small-molecule binders of OTUB1 to de-ubiquitinylate and stabilize proteins such as AMPK, cGAS and CFTR. Also disclosed are methods for the treatment of AMPK, cGAS and CFTR-mediated diseases in a subject in need thereof. Also disclosed are methods for designing such compounds including irreversible small-molecule binders of OTUB1 and related bivalent compounds as de-ubiquitinase-targeting chimeras (DUBTACs) for targeted protein stabilization.

### CROSS-REFERENCE TO RELATED APPLICATION

The present application claims priority to U.S. Provisional Application Ser. No. 63/497,651, filed on Apr. 21, 2023, the contents of which are hereby incorporated by reference in their entirety.

### STATEMENT REGARDING GOVERNMENT FUNDING

This invention was made with government support under R35CA253027 awarded by the National Institutes of Health. The government has certain rights in the invention.

### TECHNICAL FIELD

The present disclosure is directed to compounds that are irreversible small-molecule binders of OTUB1 (also known as Otubain 1), and bivalent compounds (e.g. bi-functional small molecule compounds) which recruit OTUB1 through the irreversible small-molecule binders of OTUB1 to de-ubiquitinylate and stabilize proteins such as AMPK, cGAS and CFTR. The present disclosure is also related to methods for the treatment of AMPK, cGAS and CFTR-mediated diseases in a subject in need thereof. The disclosure also relates to methods for designing such compounds including irreversible small-molecule binders of OTUB1 and related bivalent compounds as deubiquitinase-targeting chimeras (DUBTACs) for targeted protein stabilization (TPS).

### BACKGROUND

Protein ubiquitination plays a critical role in a variety of cellular process, like protein degradation, quality control, trafficking, and signaling. Therefore, controlling the ubiquitination level of disease related proteins can achieve substantial therapeutic effects. For example, inducing the ubiquitination of oncoproteins such as Myc and Ras to lead to their degradation would greatly benefit related cancer therapy. Similarly, deubiquitination and stabilization of proteins such as tumor suppressors would also be greatly beneficial for the treatment of relevant diseases including cancer.

5′ adenosine monophosphate-activated protein kinase (also known as AMPK) is an enzyme that plays key roles in cellular energy homeostasis. Recent studies identified AMPK as regulating diverse metabolic and physiological processes. However, it is downregulated in major chronic diseases, such as obesity, inflammation, diabetes and cancer. Therefore, stabilization of AMPK in the cellular context may enhance AMPK activation, and has the potential to provide a novel therapeutic strategy to treat such diseases (Jeon, 2016).

The protein stabilization strategy could also be applied to other targets, such as cyclic GMP-AMP (cGAMP) synthase (also known as cGAS). The cGAS-stimulator of interferon genes (STING) pathway is a component of the innate immune system. cGAS detects the presence of cytosolic DNA and, in response, triggers expression of inflammatory genes that can lead to senescence or to the activation of defense mechanisms. In tumor cells, the cGAS-STING pathway exerts its antitumor effects through the induction of a robust type I IFN response, which activates immune cells, particularly dendritic cells (DCs), in the tumor micro environment (TME) (Samson and Ablasser, 2022).

In addition to AMPK and cGAS, Cystic fibrosis transmembrane conductance regulator (CFTR) is another suitable protein target for stabilization. CFTR ΔF508 mutation destabilizes CFTR, and leads to the cystic fibrosis phenotype (Ward et al., 1995). Therefore, deubiquitination of ΔF508 mutant CFTR could be an effective strategy to prevent the cystic fibrosis process.

The targeted protein degradation (TPD) field has made great progress over the past 10 years. PROteolysis TArgeting Chimera (PROTAC) technology has been widely utilized in the drug discovery field (Dale et al., 2022). Deubiquitinase-targeting chimera (DUBTAC), which hijacks a cellular deubiquitinase to remove the polyubiquitin attached to the target protein by inducing close proximity between the target protein and deubiquitinase, recently emerged as an effective technology to improve target protein stability by reducing the proteasomal degradation of the target protein (Henning et al., 2022; Liu et al., 2022). DUBTACs provide a novel therapeutic approach for treating various diseases.

### SUMMARY

The present disclosure includes two parts: (A) small molecules that covalently bind the deubiquitinase OTUB1, and (B) heterobifunctional small molecules that stabilize AMPK, cGAS, or CFTR by recruiting OTUB1.

### A. OTUB1 Irreversible Small-Molecule Binders

OTUB1 (also known as OUT domain-containing ubiquitin aldehyde-binding protein1) is a K48 linkage-specific deubiquitinating enzyme. There are very few small molecules reported to bind OTUB1, including the covalent binder EN523 (Henning et al., 2022), and reversible inhibitor compound 61 (Tan et al., 2022), ML364 (WO2020223551A1). However, EN523 has low binding affinity to OTUB1, and compound 61 and ML364 inhibit the enzymatic activity of OTUB1. Therefore, development of more effective OTUB1 small-molecule binders that lead to effective DUBTACs is needed.

Described herein are small molecules that covalently modify OTUB1.

In one aspect, the present disclosure features small molecules that covalently modify OTUB1 of Formula (A-I) below:

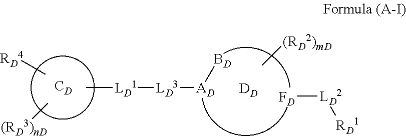

or a tautomer thereof, or a pharmaceutically acceptable salt of the compound or the tautomer,
wherein
A_{D} is selected from N, or CR_{D}².
B_{D} is selected from C═O, CH₂, CH R_{D}², or C(R_{D}²)₂;
Ring C_{D} is absent, or selected from C₃-C₁₂ cycloalkyl, 3-12-membered heterocyclic, C₆-C₁₀ aryl, and 5-10 membered heteroaryl;
Ring D_{D} is a saturated or partially unsaturated 4-12 membered heterocyclic;
F_{D} is selected from N, or CR_{D}²;
L_{D} is a bond, or a bivalent group selected from —O—, —NR_{D}⁵—, —C(O)—, —C(O)O—, —C(O)NR_{D}⁵—, —NR_{D}⁵C(O)—, —OC(O)NR_{D}⁵—, —NR_{D}⁵C(O)O—, —NR_{D}⁶C(O)NR_{D}⁵—, —S(O)—, —S(O)₂—, —S(O)NR_{D}⁵—, —S(O)₂NR_{D}⁵—, C₁-C₆ alkylene, C₁-C₆ haloalkylene, C₁-C₆ heteroalkylene, C₂-C₆ alkenylene, and C₂-C₆ alkynylene;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₆ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring C_{D}, optionally form fused rings or bridged rings;
R_{D}⁴ is R_{D}⁴a or R_{D}⁴b.
R_{D}^{4a} is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}^{4b} is a bivalent group that connect to the Linker moiety in Formula (B), and R_{D}⁴b is selected from a bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene;
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
m_{D} is an integer of 0-8; and
n_{D} is an integer of 0-8.

In some embodiments, L_{D}¹ is a bond, or a bivalent group selected from —C(O)—, —NR_{D}⁵C(O)—, and —C(O)NR_{D}⁵—. In some embodiments, L_{D}¹ is a bond. In some embodiments, L_{D}¹ is —C(O)—. In some embodiments, L_{D}¹ is —C(O)NR_{D}⁵—. In some embodiments, L_{D}¹ is —NR_{D}⁵C(O)—. In some embodiments, L_{D}³ is a bond, or a bivalent group selected from —CH₂—, or —CH₂CH₂—. In some embodiments, L_{D}³ is a bond. In some embodiments, L_{D}³ is a bond —CH₂—.

In some embodiments, the small molecules that covalently modify OTUB1 are selected from Formulae (A-I-a), (A-I-b), (A-I-c), or (A-I-d):

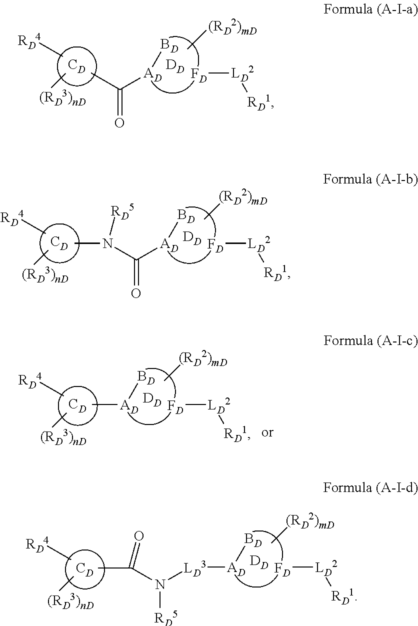

In some embodiments, A_{D} is N. In some embodiments, A_{D} is CR_{D}². In some embodiments, A_{D} in Formula (A-I-a) is N. In some embodiments, A_{D} in Formula (A-I-b) is CR_{D}². In some embodiments, A_{D} in Formula (A-I-c) is N. In some embodiments, A_{D} in Formula (A-I-d) is CR_{D}².

In some embodiments, the small molecules that covalently modify OTUB1 are selected from Formulae (A-I-a1), (A-I-b1), (A-I-c1), or (A-I-d1):

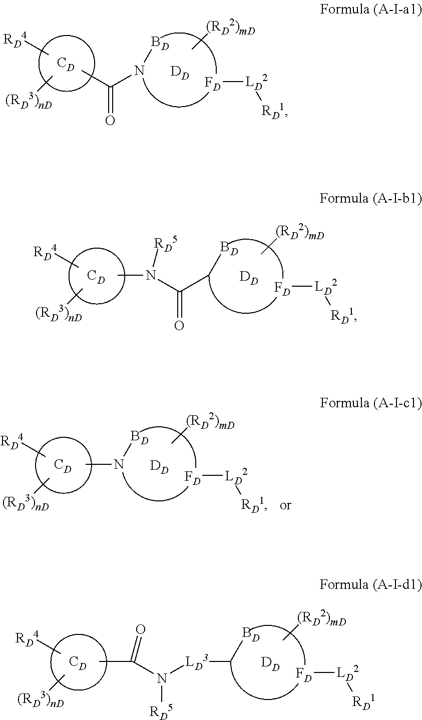

In some embodiments, the small molecules that covalently modify OTUB1 are selected from Formulae (A-I-a1), (A-I-b1), (A-I-c1), and (A-I-d1). In some embodiments, the small molecules that covalently modify OTUB1 comprise Formula (A-I-a1). In some embodiments, the small molecules that covalently modify OTUB1 comprise Formula (A-I-b1). In some embodiments, the small molecules that covalently modify OTUB1 comprise Formula (A-I-c1). In some embodiments, the small molecules that covalently modify OTUB1 comprise Formula (A-I-d1).

In some embodiments, Ring D_{D} in Formulae (A-I-a), (A-I-a1), (A-I-b1), (A-I-c1), and (A-I-d1) is selected from 6-8 membered monocyclic heterocyclic, 7-12 membered spiro heterocyclic, 7-12 membered bridged heterocyclic, and 6-12 membered fused heterocyclic.

In some embodiments, The small molecules that covalently modify OTUB1 are selected from Formulae (A-I-a2), (A-I-a3), (A-I-a4), (A-I-a5), (A-I-b2), (A-I-b3), (A-I-b4), (A-I-b5), (A-I-c2), (A-I-c3), (A-I-c4), (A-I-c5), (A-I-d2), (A-I-d3), (A-I-b4), and (A-I-d5):

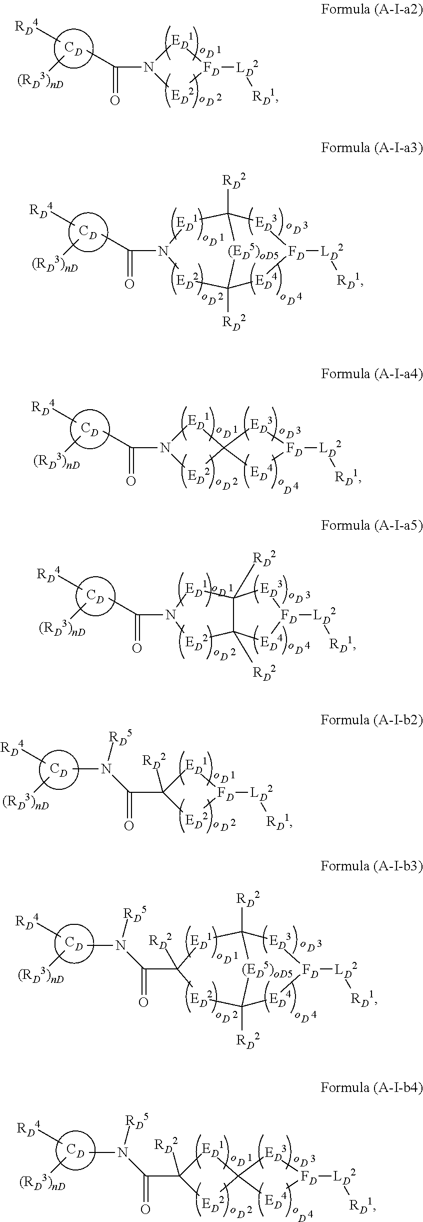

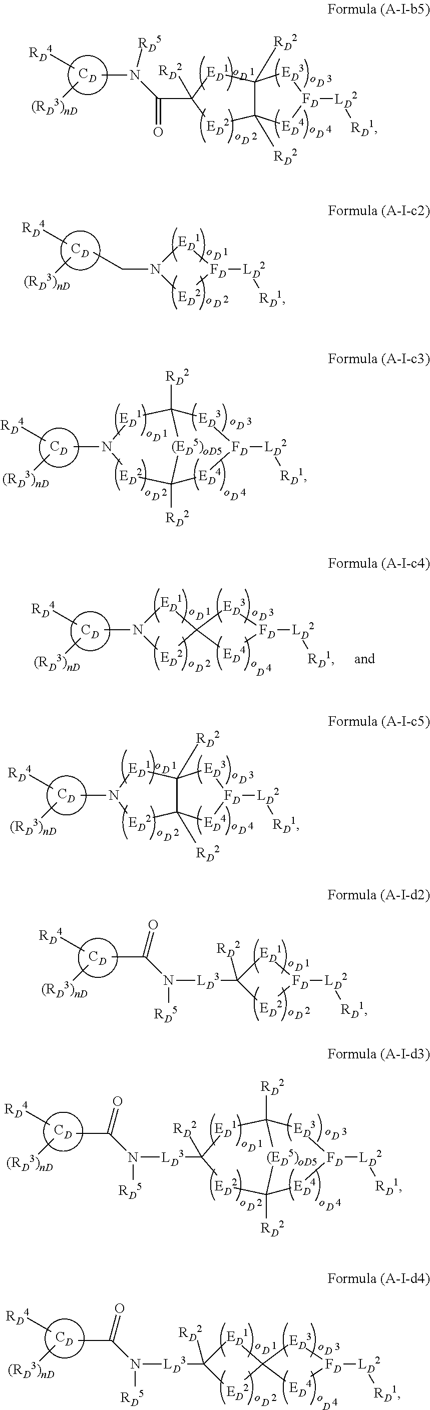

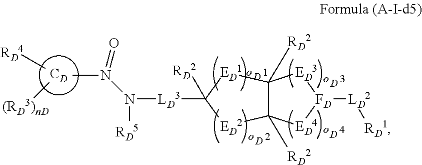

wherein
each E_{D}¹, each E_{D}², each E_{D}³, each E_{D}⁴, and eachED⁵ are independently selected from null, O, CO, SO, S(O)₂, NR_{D}², and CR_{D}²R_{D}², with the proviso that no two oxygen atoms are connected to each other;
o_{D}¹ is selected from an integer of 1-4; and
o_{D}², o_{D}³, o_{D}⁴, and o_{D}⁵ are independent selected from an integer of 0-4.

In some embodiments, the small molecules that covalently modify OTUB1 comprise Formulae (A-I-a3), (A-I-b2), and (A-I-c4). In some embodiments, the small molecules that covalently modify OTUB1 comprise Formula (A-I-a3). In some embodiments, the small molecules that covalently modify OTUB1 comprise Formula (A-I-b2). In some embodiments, the small molecules that covalently modify OTUB1 comprise Formula (A-I-c4).

In some embodiments, each E_{D}¹, each E_{D}², each E_{D}³, each E_{D}⁴, and each E_{D}⁵ are independently selected from null, CO, and CR_{D}²R_{D}². In some embodiments, each E_{D}¹, each E_{D}², each E_{D}³, each E_{D}⁴, and each E_{D}⁵ are independently selected from CO and CR_{D}²R_{D}².

In some embodiments, E_{D}¹, E_{D}², E_{D}³, E_{D}⁴, and E_{D}⁵ in Formulae (A-I-a2), (A-I-a3), (A-I-a4), (A-I-a5), (A-I-b2), (A-I-b3), (A-I-b4), (A-I-b5), (A-I-c2), (A-I-c3), (A-I-c4), (A-I-c5), (A-I-d2), (A-I-d3), (A-I-b4), and (A-I-d5) are CR_{D}²R_{D}².

In some embodiments, the small molecules that covalently modify OTUB1 are selected from Formulae (A-I-a6), (A-I-a7), (A-I-a8), (A-I-a9), (A-I-b6), (A-I-b7), (A-I-b8), (A-I-b9), (A-I-c6), (A-I-c7), (A-I-c8), (A-I-c9), (A-I-d6), (A-I-d7), (A-I-d8), and (A-I-d9):

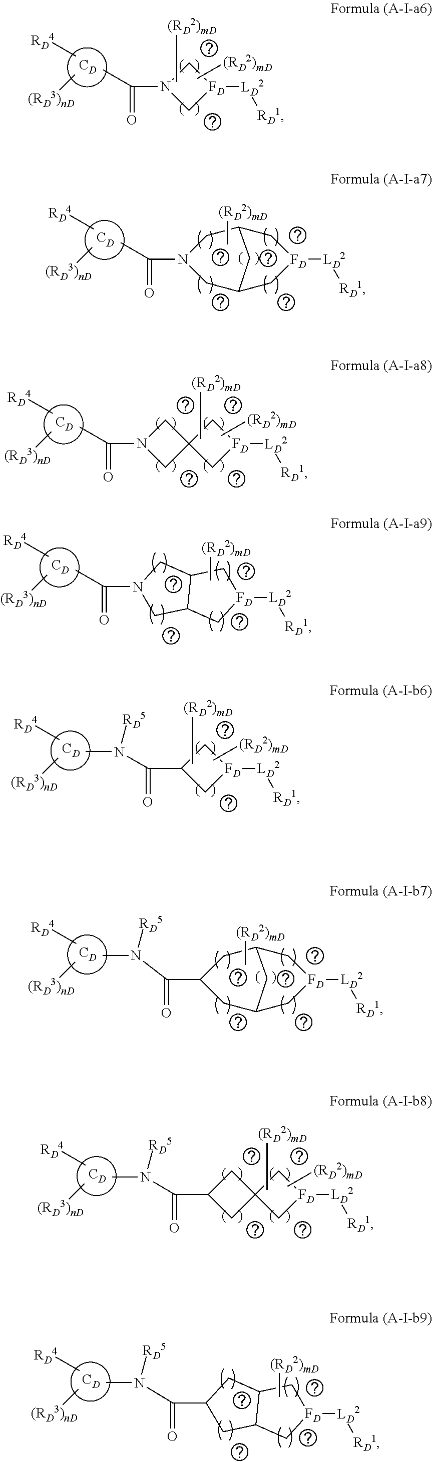

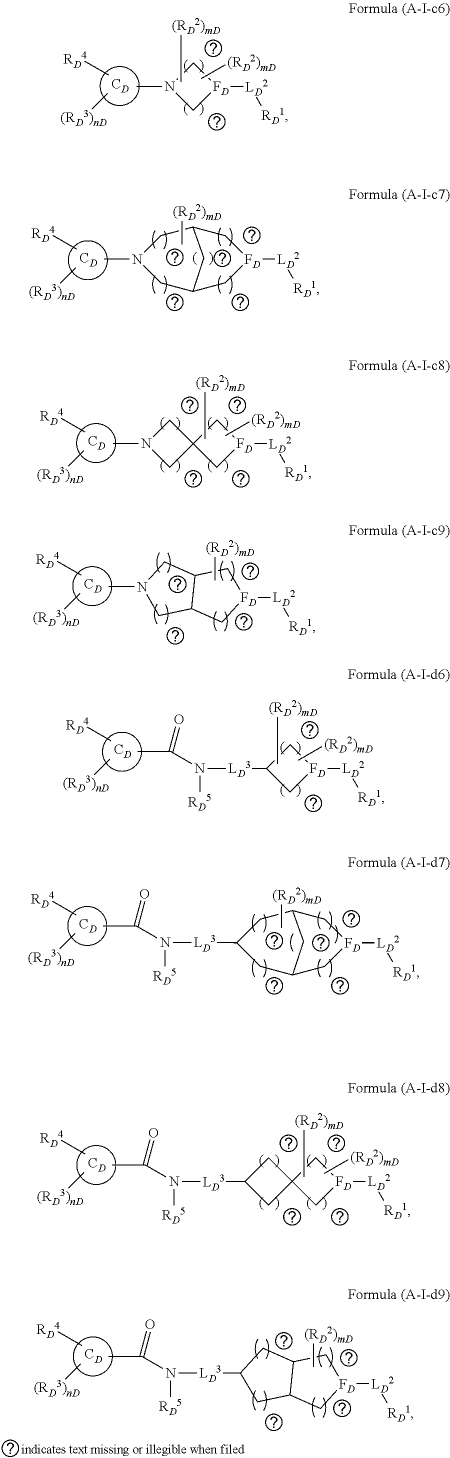

wherein
R_{D}² is a substituent that can attach to anywhere on the monocyclic and bicyclic rings.

In some embodiments, F_{D} is Selected from N, CH, or CMe. In some embodiments, F_{D} is selected from N. In some embodiments, F_{D} is selected from CH. In some embodiments, F_{D} is selected from CMe.

In some embodiments, L_{D}³ is selected from a bond or —CH₂—. In some embodiments, L_{D}³ is selected from a bond. In some embodiments, L_{D}³ is selected from —CH₂—.

In some embodiments, the small molecules that covalently modify OTUB1 comprise Formulae (A-I-a6), (A-I-a7), (A-I-b6), (A-I-c8), and (A-I-d6). In some embodiments, the small molecules that covalently modify OTUB1 comprise Formula (A-I-a6). In some embodiments, the small molecules that covalently modify OTUB1 comprise Formula (A-I-a7). In some embodiments, the small molecules that covalently modify OTUB1 comprise Formula (A-I-b6). In some embodiments, the small molecules that covalently modify OTUB1 comprise Formula (A-I-c8). In some embodiments, the small molecules that covalently modify OTUB1 comprise Formula (A-I-d6).

In some embodiments, B_{D} is selected from C═O and C(R_{D}²)₂. In some embodiments, B_{D} is C═O. In some embodiments, B_{D} is C(R_{D}²)₂.

In some embodiments, Ring D_{D} in Formulae (A-I), (A-I-a), (A-I-a1), (A-I-b), (A-I-b1), (A-I-c), (A-I-c1), (A-I-d), and (A-I-d1) is selected from Ring D_{D}^{a1}, Ring D_{D}^{a2}, Ring D_{D}^{b1}, Ring D_{D}^{b2} Ring D_{D}^{c1}, and Ring D_{D}^{c2}.

In some embodiments, Ring DD in Formulae (A-I-a), (A-I-a1), (A-I-c), and (A-I-c1) is Ring D_{D}^{a1} comprising R_{D}² substituted groups of:

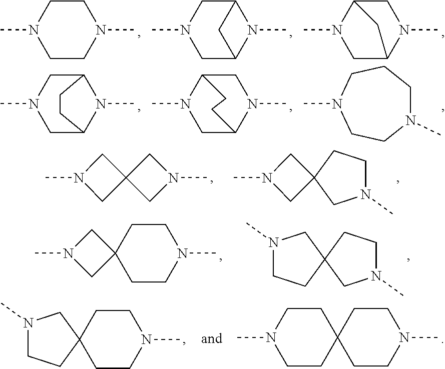

In some embodiments, Ring D_{D} in Formulae (A-I-a), (A-I-a1), (A-I-c), and (A-I-c1) is Ring D_{D}^{a2} comprising R_{D}² substituted groups of:

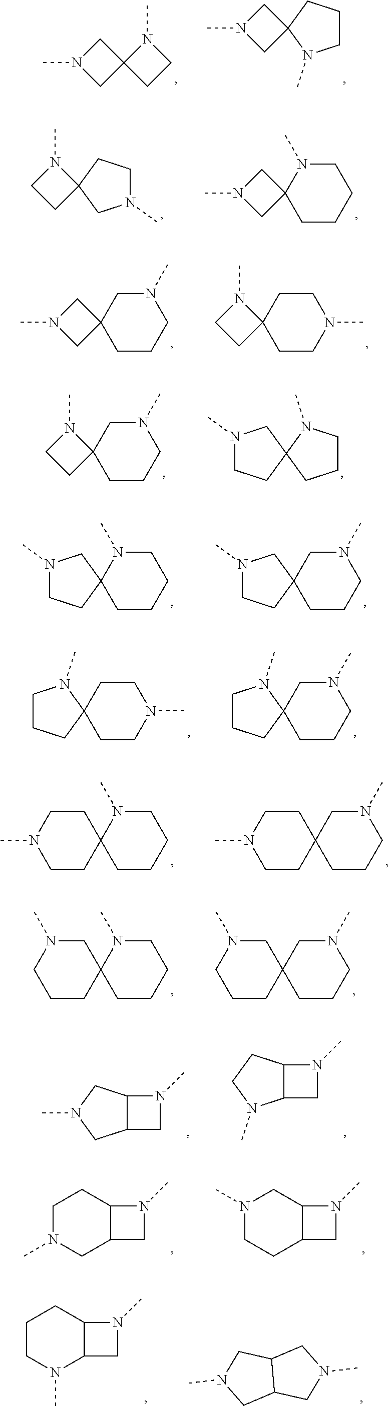

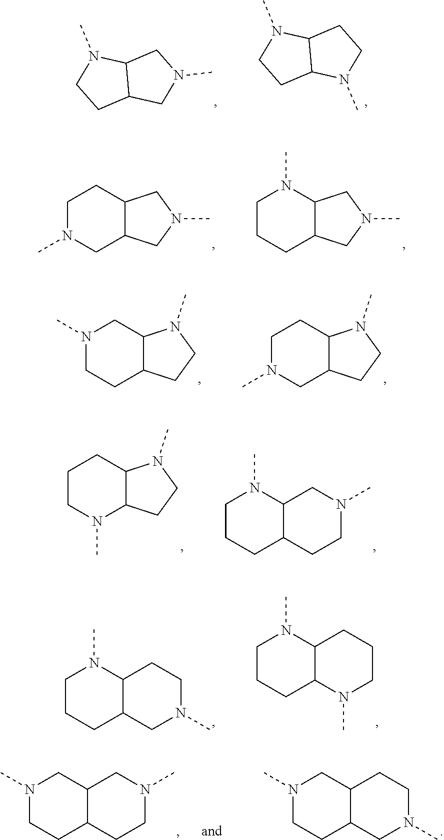

In some embodiments, Ring DD in Formulae (A-I-a) and (A-I-a1) is selected from

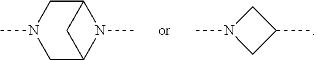

In some embodiments, Ring DD in Formulae (A-I-a) and (A-I-a1) is

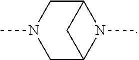

In some embodiments, Ring D_{D} in Formulae (A-I-a) and (A-I-a1) is

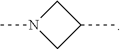

In some embodiments, the small molecules that covalently modify OTUB1 comprise Formula (A-I-a10):

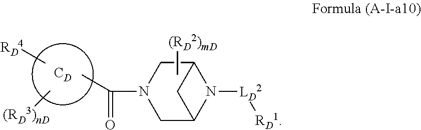

In some embodiments, the small molecules that covalently modify OTUB1 comprise Formula (A-I-a11):

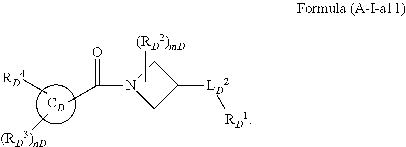

In some embodiments, Ring D_{D} in Formulae (A-I-c) and (A-I-c1) is

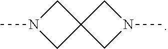

The small molecules that covalently modify OTUB1 comprise Formula (A-I-c10):

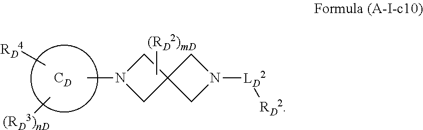

In some embodiments, Ring D_{D} in Formulae (A-I-b), (A-I-b1), (A-I-d), and (A-I-d1) is Ring D_{D}^{b1} comprising R_{D}² substituted groups of:

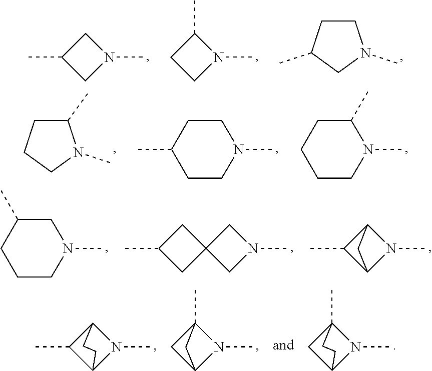

In some embodiments, Ring DD in Formulae (A-I-b), (A-I-b1), (A-I-d), and (A-I-d1), is Ring D_{D}^{b2} comprising R_{D}² substituted groups of:

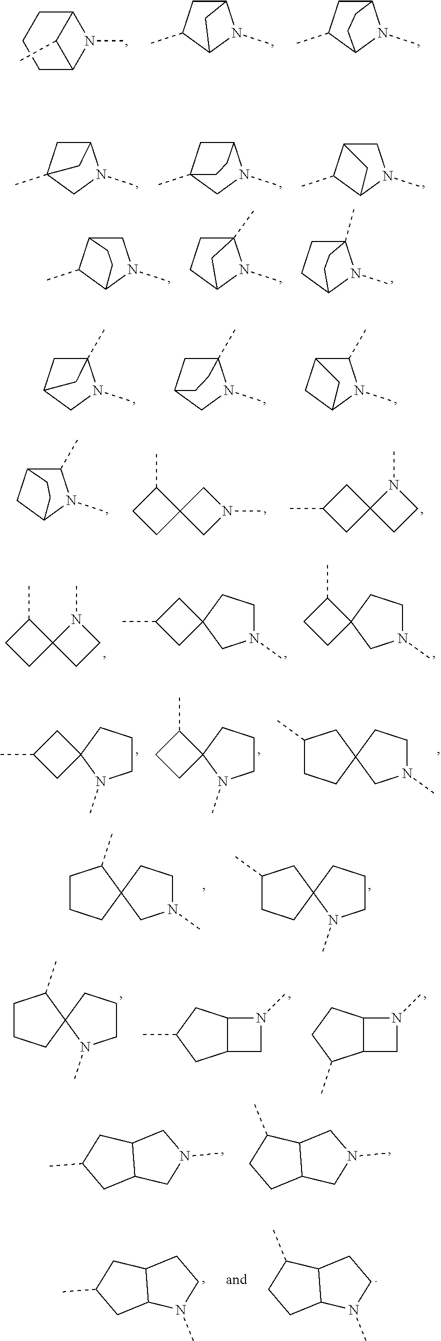

In some embodiments, Ring D_{D} in Formulae (A-I-b), (A-I-b1), (A-I-d), and (A-I-d1) is

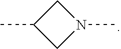

In some embodiments, the small molecules that covalently modify OTUB1 comprise Formula (A-I-b10):

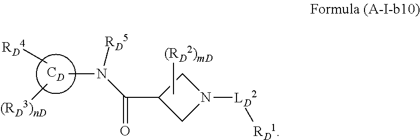

In some embodiments, the small molecules that covalently modify OTUB1 comprise Formula (A-I-d10):

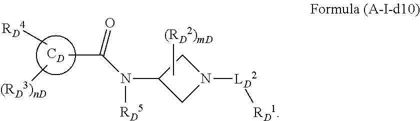

In some embodiments, Ring D_{D} in Formulae (A-I-b), (A-I-b1), (A-I-d), and (A-I-d1) is Ring D_{D}^{d1} comprising R_{D}² substituted groups of:

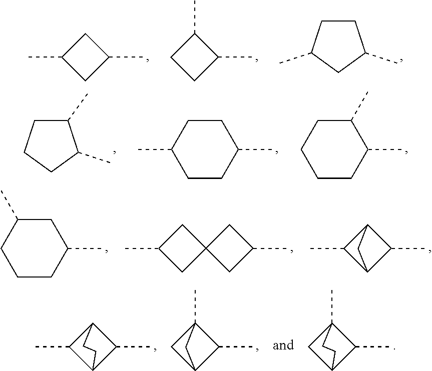

In some embodiments, Ring D_{D} in Formulae (A-I-b), (A-I-b1), (A-I-d), and (A-I-d1), is Ring D_{D}^{b2} comprising R_{D}² substituted groups of:

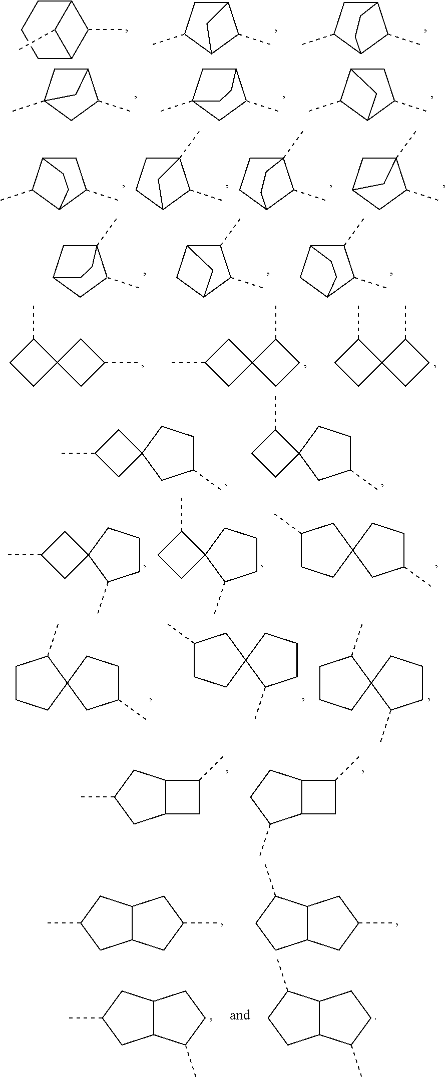

In some embodiments, Ring DD in Formulae (A-I-b), (A-I-b1), (A-I-d), and (A-I-d1) is

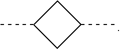

In some embodiments, the small molecules that covalently modify OTUB1 comprise Formula (A-I-b11):

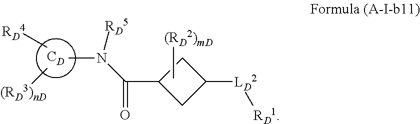

In some embodiments, the small molecules that covalently modify OTUB1 comprise Formula (A-I-d11):

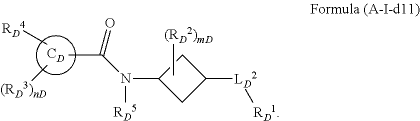

In some embodiments, E_{D}¹ in Formulae (A-I-c2), (A-I-c3), (A-I-c4), and (A-I-c5) that is adjacent to N atom is —C(O)—, and the rest of the E_{D}¹, E_{D}², E_{D}³, E_{D}⁴, and E_{D}⁵ are CR_{D}²R_{D}². The small molecules that covalently modify OTUB1 comprise Formulae (A-I-c11), (A-I-c12), (A-I-c13), and (A-I-c14):

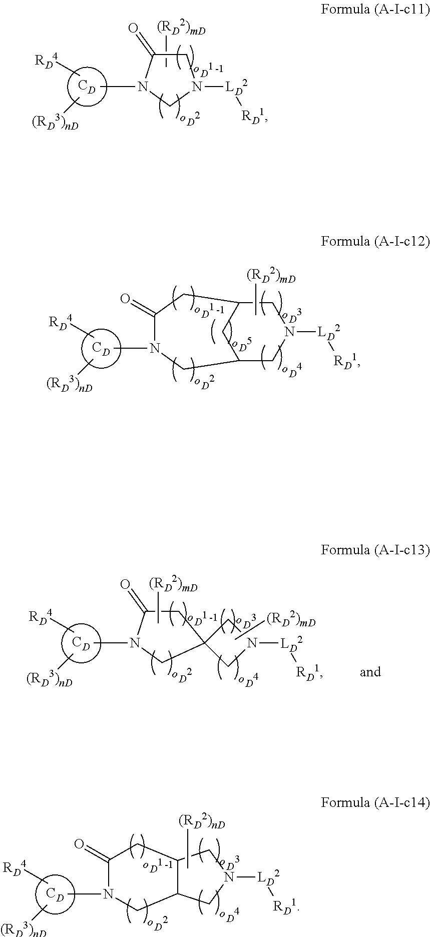

wherein
o_{D}¹-1 is selected from an integer of 0-3.

In some embodiments, the small molecules that covalently modify OTUB1 comprise Formulae (A-I-c11) and (A-I-c12). In some embodiments, the small molecules that covalently modify OTUB1 comprise Formulae (A-I-c11). In some embodiments, the small molecules that covalently modify OTUB1 comprise Formulae (A-I-c12).

In some embodiments, Ring D_{D} in Formulae (A-I-c) and (A-I-c1) is Ring D_{D}C₁ comprising R_{D}² substituted groups of:

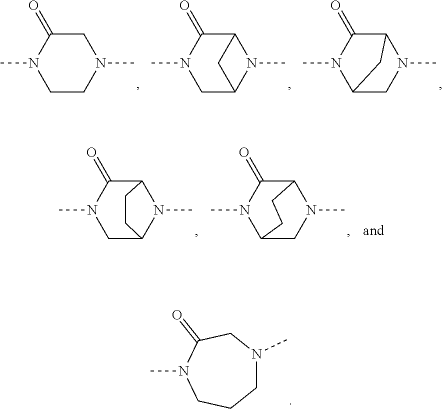

In some embodiments, Ring D_{D} in Formulae (A-I-c) and (A-I-c1) is Ring D_{D}^{c2} comprising R_{D}² substituted groups of

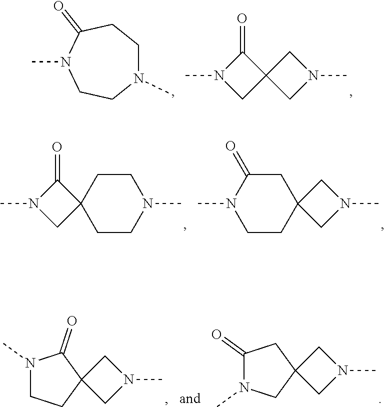

In some embodiments, Ring D_{D} in Formulae (A-I-c) and (A-I-c1) is

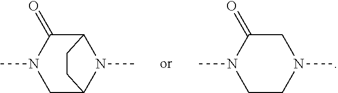

In some embodiments, the small molecules that covalently modify OTUB1 comprise Formulae (A-I-c15) and (A-I-c16):

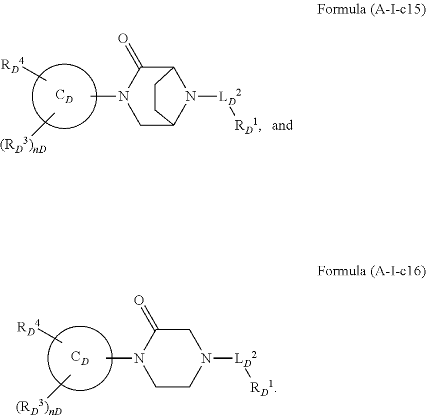

In some embodiments, Ring C_{D} is selected from C₃-C₁₂ cycloalkyl, 3-12-membered heterocyclic, C₆-C₁₀ aryl, and 5-10 membered heteroaryl. In some embodiments, Ring C_{D} is C₃-C₁₂ cycloalkyl. In some embodiments, Ring C_{D} is selected from cyclopropyl, cyclobutyl, cyclopentyl, cyclohexyl, and cycloheptyl. In some embodiments, Ring C_{D} is cyclopentyl. In some embodiments, Ring C_{D} is 3-12-membered heterocyclic. In some embodiments, Ring C_{D} is selected from azetidinyl, pyrrolidinyl, piperidinyl, and piperazinyl. In some embodiments, Ring C_{D} is C₆-C₁₀ aryl. In some embodiments, Ring C_{D} is phenyl. In some embodiments, Ring C_{D} is 5-10 membered heteroaryl. In some embodiments, Ring C_{D} is selected from thiophenyl, benzothiophenyl, tetrahydrobenzothiophenyl, thiazolyl, imidazolyl, furanyl, pyrrolyl, pyrazolyl, pyridinyl, pyrimidinyl, pyridazinyl, pyrazinyl, benzofuranyl, indolyl, and indazolyl. In some embodiments, Ring C_{D} is selected from thiophenyl, benzothiophenyl, tetrahydrobenzothiophenyl, benzofuranyl, and indolyl. In some embodiments, Ring C_{D} is thiophenyl. In some embodiments, Ring C_{D} is

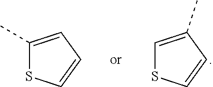

In some embodiments, Ring C_{D} is

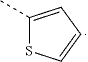

In some embodiments, Ring C_{D} is benzothiophenyl. In some embodiments, Ring C_{D} is

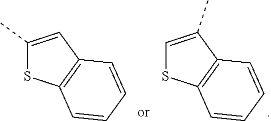

In some embodiments, Ring C_{D} is

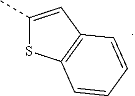

In some embodiments, Ring C_{D} is tetrahydrobenzothiophenyl. In some embodiments, Ring C_{D} is or

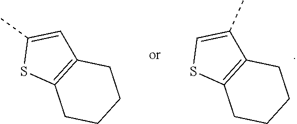

In some embodiments, Ring C_{D} is

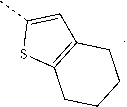

In some embodiments, Ring C_{D} is

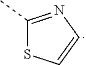

In some embodiments, Ring C_{D} is

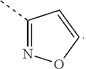

In some embodiments, L_{D}² is selected from —C(O)—, C₁-C₆ alkylene, and —NR_{D}⁵C(O)—. In some embodiments, L_{D}² is selected from —C(O)—, —NHC(O)—, and methylene. In some embodiments, L_{D}² is —C(O)—. In some embodiments, —NHC(O)—. L_{D}² is In some embodiments, L_{D}² is methylene.

In some embodiments, L_{D}² is —C(O)—; and R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}^{B}, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic. In some embodiments, L_{D}² is C₁-C₆ alkylene; and R_{D}¹ is selected from C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}^{B}, C(O)OR_{D}⁷, tri(C₁-C₃alkyl) silyl, C₁-C₆ alkyl, C₁-C₆ heteroalkyl, and 3-8 membered heterocyclic. In some embodiments, L_{D}² is methylene; and R_{D}¹ is selected from C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic.

In some embodiments, L_{D}² is —NHC(O)—; and R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic. In some embodiments, L_{D}² is C₁-C₆ alkylene; and R_{D}¹ is selected from C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic. In some embodiments, L_{D}² is methylene; and R_{D}¹ is selected from C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic.

In some embodiments, R_{D}¹ is C₁-C₆ haloalkyl. In some embodiments, R_{D}¹ is halomethyl. In some embodiments, R_{D}¹ is CH₂Cl or CH₂F. In some embodiments, R_{D}¹ is selected from C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic. In some embodiments, R_{D}¹ is selected from C₂-C₃ alkenyl, C₂-C₃ alkynyl, partially unsaturated C₄-C₆ cycloalkyl, and partially unsaturated 4-6 membered heterocyclic, where each said C₂-C₆ alkenyl, C₂-C₆ alkynyl, partially unsaturated C₄-C₆ cycloalkyl, and partially unsaturated 4-6 membered heterocyclic were optionally substituted with hydrogen, halogen, cyano, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic. In some embodiments, R_{D}¹ is selected from vinyl, propylenyl, ethynyl, propargyl, cyclobutenyl, cyclopentenyl, cyclohexenyl, dihydrofuranyl, dihydropyrrolyl, dihydropyranyl, and tertrahydopyridinyl, where each said vinyl, propylenyl, ethynyl, propargyl, cyclobutenyl, cyclopentenyl, cyclohexenyl, dihydrofuranyl, dihydropyrrolyl, dihydropyranyl, and tertrahydopyridinyl were optionally substituted with hydrogen, halogen, cyano, NR_{D}⁷R_{D}⁸, C(O)NR_{D}⁷R_{D}⁸, C(O)OR_{D}⁷, trimethyl silyl, C₁-C₃ alkyl, C₁-C₃ heteroalkyl, and 3-6 membered heterocyclic. In some embodiments, R_{D}¹ is selected from vinyl and propylenyl, where each said vinyl and propylenyl were optionally substituted with hydrogen, halogen, cyano, NR_{D}⁷R_{D}⁸, C(O)NR_{D}⁷R_{D}⁸, C(O)OR_{D}⁷, trimethyl silyl, C₁-C₃ alkyl, C₁-C₃ heteroalkyl, and 3-6 membered heterocyclic. In some embodiments, R_{D}¹ is selected from

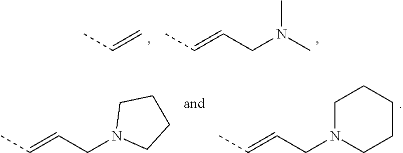

In some embodiments, R_{D}¹ is

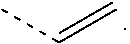

In some embodiments, R_{D}¹ is

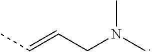

In some embodiments, -L_{D}²-R_{D}¹ is selected from

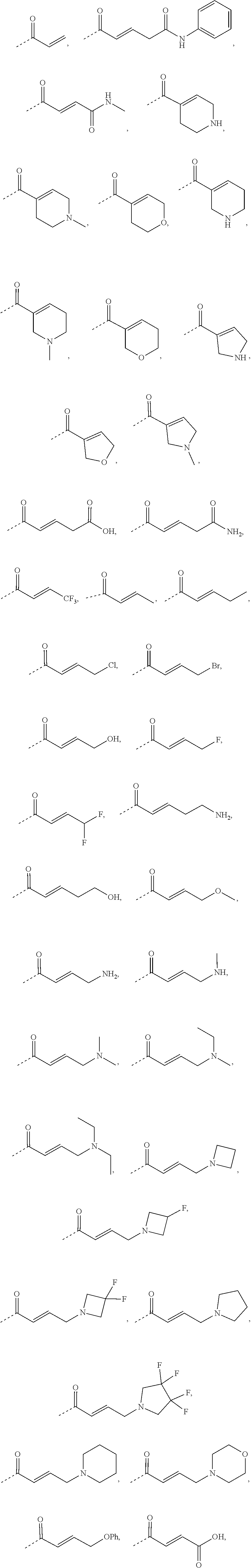

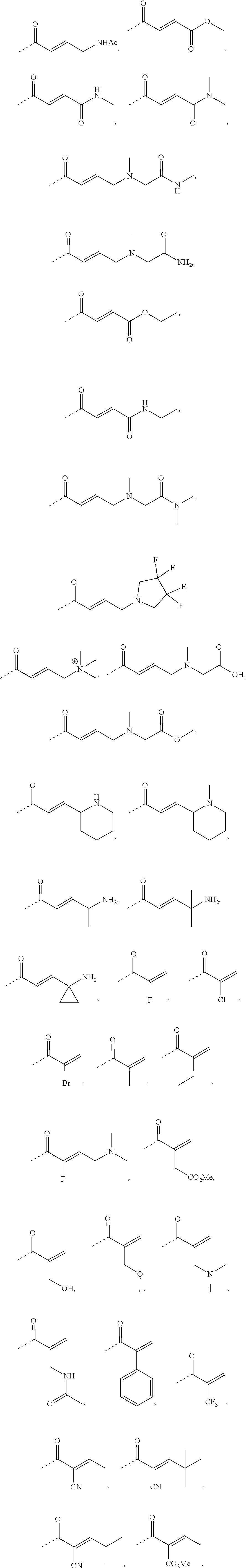

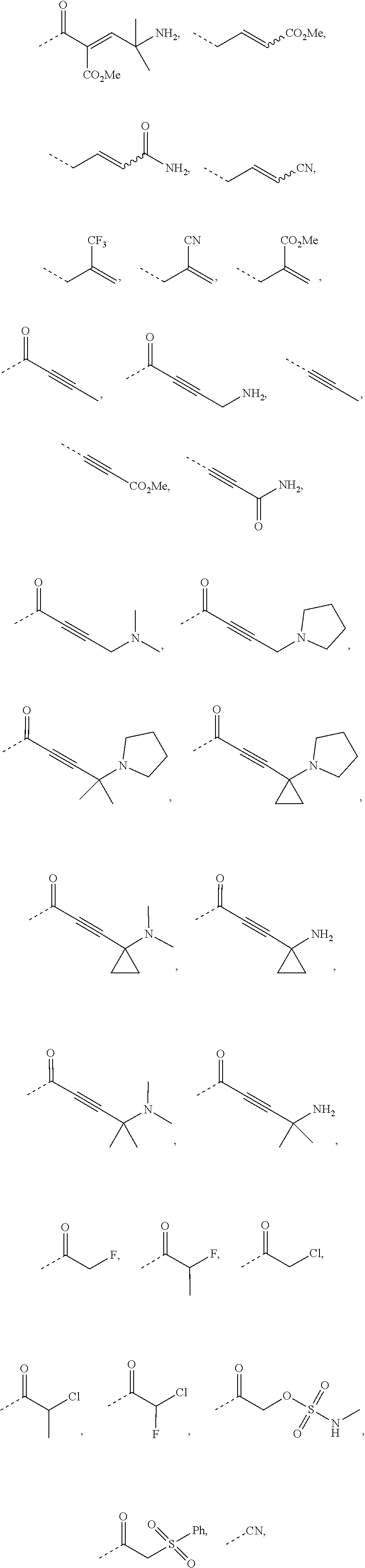

In some embodiments, -L_{D}²-R_{D}¹ is selected from

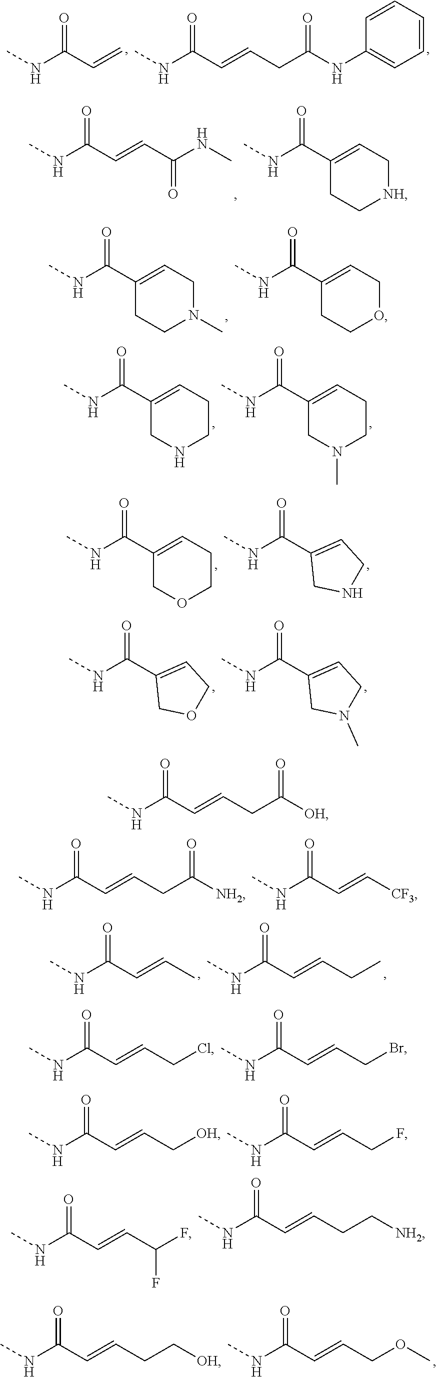

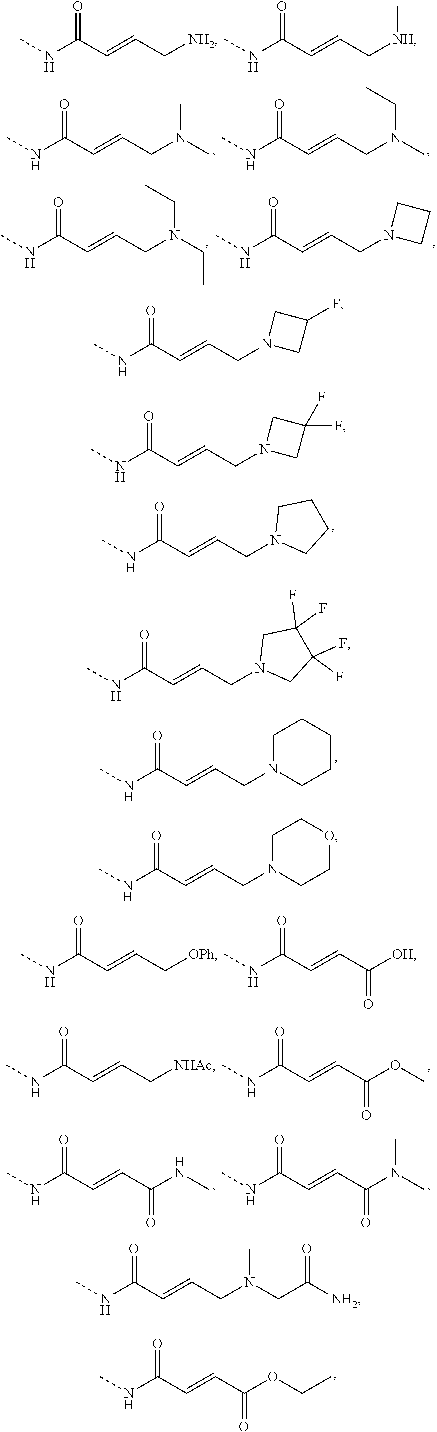

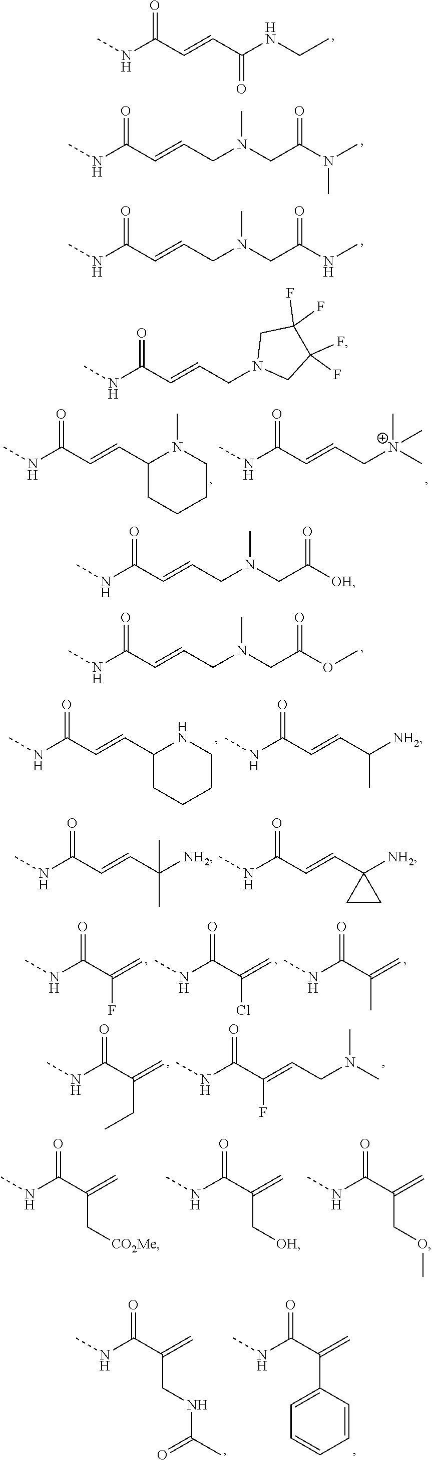

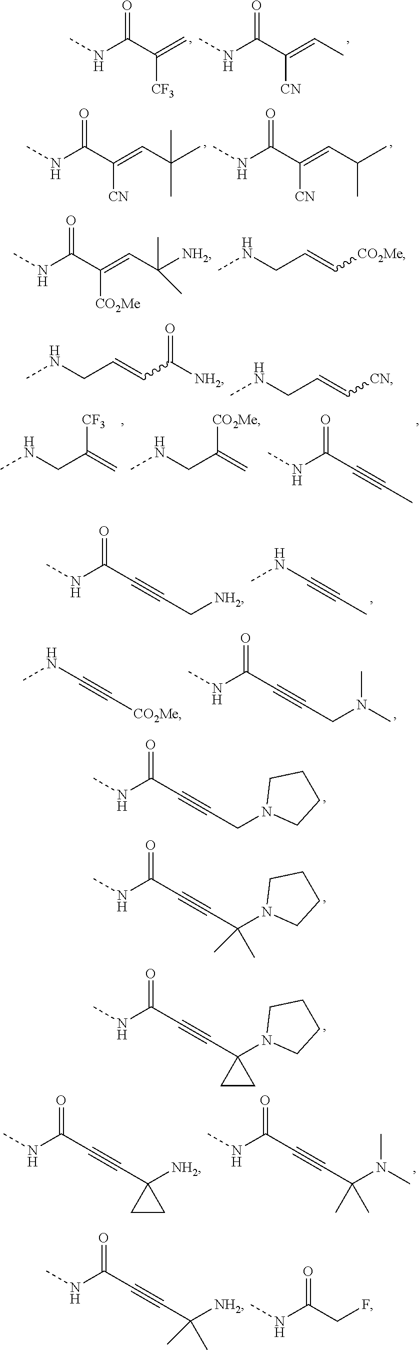

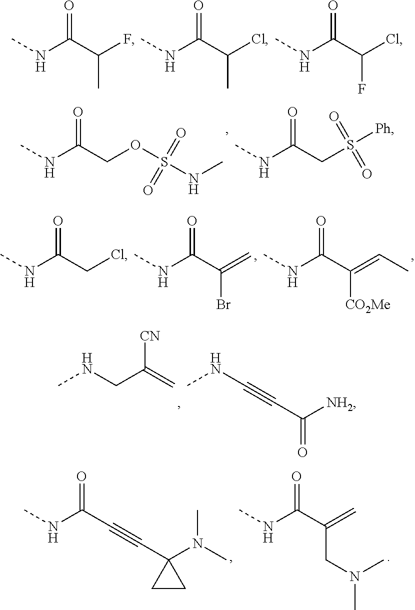

In some embodiments, -L_{D}²-R_{D}¹ is selected from

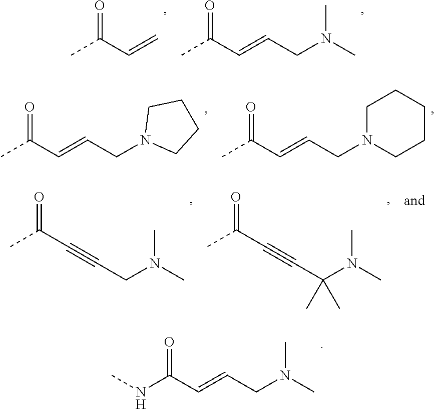

In some embodiments, -L_{D}²-R_{D}¹ is

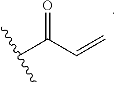 [SMILES: *C(=O)C=C]

In some embodiments, -L_{D}²-R_{D}¹ is

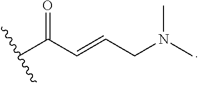 [SMILES: *C(=O)/C=C/CN(C)C]

In some embodiments, -L_{D}²-R_{D}¹ is

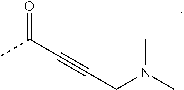

In some embodiments, -L_{D}²-R_{D}¹ is

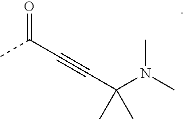

In some embodiments, -L_{D}²-R_{D}¹ is

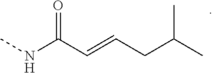

In some embodiments, each R_{D}² is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic. In some embodiments, each R_{D}² is selected from hydrogen, halogen, hydroxy, C₁-C₆ alkoxy, amino, C₁-C₆ alkyl amino, (C₁-C₆ alkyl)(C₁-C₆ alkyl)amino, C₁-C₆ alkyl, C₁-C₆ haloalkyl, C₁-C₆ heteroalkyl, C₃-C₈ cycloalkyl, 3-8 membered heterocyclic. In some embodiments, each R_{D}² is selected from hydrogen, halogen, C₁-C₆ alkyl, C₁-C₆ haloalkyl, C₃-C₈ cycloalkyl, 3-8 membered heterocyclic. In some embodiments, R_{D}² is hydrogen. In some embodiments, R_{D}² is halogen. In some embodiments, R_{D}² is C₁-C₆ alkyl.

In some embodiments, two R_{D}² groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring D_{D}, can optionally form fused rings or bridged rings. In some embodiments, two R_{D}² groups, together with the atom(s) to which they are connected and Ring D_{D}, optionally form a bridged 3-12-membered heterocyclic ring. In some embodiments, two R_{D}² groups, together with the atom(s) to which they are connected and Ring D_{D}, optionally form a bridged 7-membered heterocyclic ring. In some embodiments, two R_{D}² groups, together with the atom(s) to which they are connected and Ring D_{D}, optionally form a bridged 8-membered heterocyclic ring.

In some embodiments, each R_{D}³ is independently selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl. In some embodiments, each R_{D}³ is selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₃-C₁₂ cycloalkyl, 3-12-membered heterocyclic, C₆-C₁₀ aryl, and 5-10 membered heteroaryl. In some embodiments, each R_{D}³ is selected from methyl, ethyl, isopropyl, piperidinyl, piperazinyl, and phenyl.

In some embodiments, two R_{D}³ groups, together with the atom(s) to which they are connected, optionally form a C₃-C₁₂ cycloalkyl ring, or 3-12-membered heterocyclic ring, where each said C₃-C₁₂ cycloalkyl ring or 3-12-membered heterocyclic ring, together with Ring C_{D}, optionally form fused rings or bridged rings.

In some embodiments, R_{D}⁴ is R_{D}^{4a}. In some embodiments, R_{D}^{4a} is selected from hydrogen, OR_{D}⁷, NR_{D}⁷R_{D}⁸, C(O)NR_{D}⁷R_{D}⁸, S(O)₂NR_{D}⁷R_{D}⁸, C₁-C₆ alkylene, C₁-C₆ haloalkylene, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl. In some embodiments, R_{D}⁴ is R_{D}^{4b}. In some embodiments, R_{D}⁴ is a bivalent group that connect to the Linker moiety in Formula (B), and R_{D}^{4b} is selected from a bond, —O—, —N—, —C(O)—, —C(O)O—, —C(O)NR_{D}⁷—, —S(O)₂—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene. In some embodiments, R_{D}⁴ is a bond.

In some embodiments, each R_{D}⁵ and each R_{D}⁶ are independently selected from hydrogen, C₁-C₆ alkyl, C₁-C₆ haloalkyl, C₁-C₆ heteroalkyl, C₃-C₁₂ cycloalkyl, and 3-12 membered heterocyclic.

In some embodiments, each R_{D}⁷, each R_{D}⁸ and each R_{D}⁹ are independently selected from hydrogen, C₁-C₆ alkyl, C₁-C₆ haloalkyl, C₁-C₆ heteroalkyl, C₃-C₁₂ cycloalkyl, and 3-12 membered heterocyclic.

In some embodiments, R_{D}⁷ and R_{DR}, together with the atom(s) to which they are connected, optionally form 4-6-membered heterocyclic ring.

In some embodiments, m_{D} is an integer of 0-4. In some embodiments, m_{D} is an integer of 1-3. In some embodiments, o_{D}¹ is selected from an integer of 1-3. In some embodiments, o_{D}² is selected from an integer of 0-3. In some embodiments, o_{D}³ is selected from an integer of 0-3. In some embodiments, o_{D}⁴ is selected from an integer of 0-3. In some embodiments, o_{D}⁵ is selected from an integer of 0-3.

In some embodiments, the small molecules that covalently modify OTUB1 are selected from the structures of Formulae (A-I), (A-I-a), (A-I-a1), (A-I-a2), (A-I-a3), (A-I-a4), (A-I-a5), (A-I-a6), (A-I-a7), (A-I-a8), (A-I-a9), (A-I-a10), (A-I-a11), (A-I-b), (A-I-b1), (A-I-b2), (A-I-b3), (A-I-b4), (A-I-b5), (A-I-b6), (A-I-b7), (A-I-b8), (A-I-b9), (A-I-b10), (A-I-b11), (A-I-c), (A-I-c1), (A-I-c2), (A-I-c3), (A-I-c4), (A-I-c5), (A-I-c6), (A-I-c7), (A-I-c8), (A-I-c9), (A-I-c10), (A-I-c11), (A-I-c12), (A-I-c13), (A-I-c14), (A-I-c15), (A-I-c16), (A-I-d), (A-I-d1), (A-I-d2), (A-I-d3), (A-I-d4), (A-I-d5), (A-I-d6), (A-I-d7), (A-I-d8), (A-I-d9), (A-I-d10), and (A-I-d11), wherein R_{D}⁴ is R_{D}^{4a} In some embodiments, the small molecules that covalently modify OTUB1 are selected from the structures of Formulae (A-I), (A-I-a), (A-I-a1), (A-I-a2), (A-I-a3), (A-I-a4), (A-I-a5), (A-I-a6), (A-I-a7), (A-I-a8), (A-I-a9), (A-1-a10), (A-I-a11), (A-I-b), (A-I-b1), (A-I-b2), (A-I-b3), (A-I-b4), (A-I-b5), (A-I-b6), (A-I-b7), (A-I-b8), (A-I-b9), (A-I-b10), (A-I-b11), (A-I-c), (A-I-c1), (A-I-c2), (A-I-c3), (A-I-c4), (A-I-c5), (A-I-c6), (A-I-c7), (A-I-c8), (A-I-c9), (A-I-c10), (A-I-c11), (A-I-c12), (A-I-c13), (A-I-c14), (A-I-c15), (A-I-d), (A-I-d1), (A-I-d2), (A-I-d3), (A-I-d4), (A-I-b5), (A-I-b6), (A-I-d7), (A-I-d8), (A-I-d9), (A-I-b10), (A-I-d11),wherein R_{D}⁴ is R_{D}^{4a}; and exclude

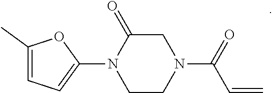 [SMILES: C=CC(=O)N1CCN(c2ccc(C)o2)C(=O)C1]

In some embodiments, the small molecules that covalently modify OTUB1 are selected from XS154-91, XS154-130, XS154-148, XS154-114, XS154-149, XS159-13, XS159-107, XS165-30, XS159-90, XS165-53, XS165-38, XS165-33, XS165-54, XS154-184, XS165-75, XS165-77, XS165-127, XS165-106, XS165-112, XS165-97, XS165-118, XS165-110, XS165-119, XS165-123, XS165-126, XS165-120, XS165-121, XS165-100, XS165-99, XS165-113, XS165-109, XS165-117, XS165-105, XS165-154, XS165-170, XS165-155, XS165-172, XS165-169, XS165-177, XS175-45, XS175-46, XS175-59, XS175-63, XS175-64, XS175-67, XS175-68, XS175-70, XS175-71, XS175-76, XS175-110, XS175-120, XS175-126, XS175-132, XS175-133, XS175-137, XS175-143, XS175-148, XS175-149, XS175-158, XS175-159, XS175-160, XS175-173, XS175-174, XS175-175, XS175-176, XS175-178, XS175-179, XS175-180, XS175-186, XS186-3, XS185-4, XS186-5, XS185-6, XS185-24, XS185-29, XS185-43, XS185-46, XS185-59, XS185-64, XS185-65, XS185-66, XS185-67, XS185-69, XS185-70, XS185-71, XS185-72, XS185-75, XS185-78, XS185-86, XS185-87, XS185-90, 91, XS185-96, XS185-97, XS185-101, XS185-109, XS185-113, XS185-114, XS185-116, XS185-118, XS185-122, XS185-131, XS185-134, XS185-135, XS185-138, XS185-140, XS185-147, XS185-149, XS185-150, XS185-171, XS190-9, XS190-27, XS190-38, XS190-44, XS190-45, XS190-46, XS190-47, XS190-48, XS190-49, XS190-50, XS190-59, XS190-68, XS190-69, XS190-70, XS190-75, XS190-75-2, XS190-77, XS190-80, XS190-81, XS190-126, XS190-127, XS190-128, XS190-129, XS190-130, XS190-137, XS190-138, XS190-144, XS190-157, XS190-158, XS190-170, XS190-181, XS190-182, XS190-183, XS190-186, XS197-3, XS197-4, XS197-5, XS197-6, XS197-14, XS197-29, XS197-38, XS197-49, XS197-50, XS197-70, XS197-71, XS197-72, XS197-73, XS197-74, XS197-85, XS197-93, XS197-94, XS197-96, XS197-133, XS197-145, XS197-176, XS197-185, XS197-186, XS209-21, XS209-22, XS209-23, XS209-24, XS209-25, XS209-26, XS209-27, XS209-28, XS209-30, XS209-38, XS209-39, XS209-40, XS209-53, XS209-54, XS209-55, XS209-57, XS209-60, XS209-62, XS209-65, XS209-74, XS209-75, XS209-92, XS209-99, XS209-100, XS209-101, XS209-122, XS209-139, XS209-140, XS209-165, XS209-166, XS209-167, XS209-168, XS209-174, XS209-175, XS209-176, XS209-184, XS209-185, XS224-6, XS224-106, XS224-107, XS224-108, XS224-109, XS224-110, XS224-111, XS224-116, XS224-117, XS224-118, XS224-119, XS224-143, XS224-144, XS224-145, XS224-147, XS224-148, XS224-149, XS224-150, XS224-154, XS224-155, XS224-156, XS224-157, XS224-158, XS224-159, or analogs thereof.

In some embodiments, the small molecules that covalently modify OTUB1 are

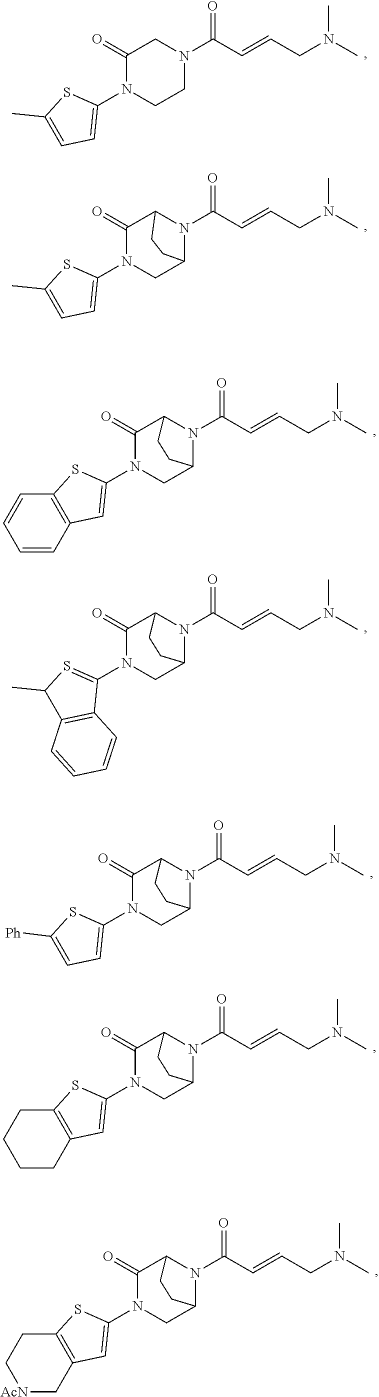

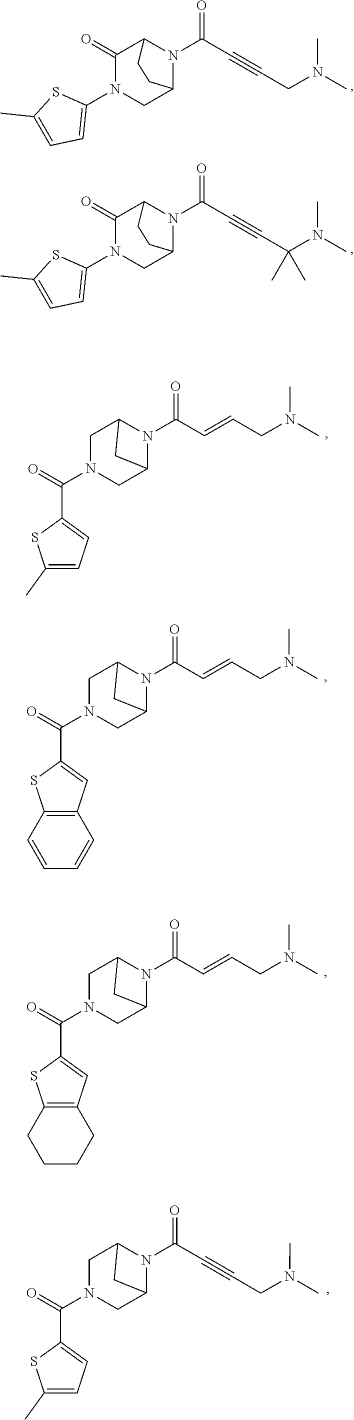

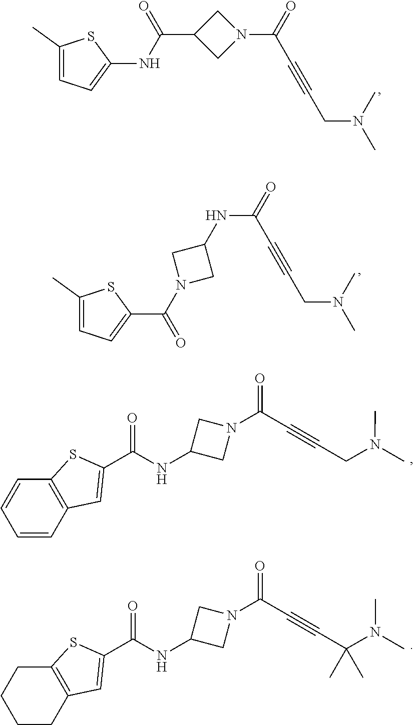

### B. OTUB1-Recruiting DUBTACs

The ubiquitination and degradation of many proteins is the cause of several classes of diseases. Therefore, de-ubiquitination and stabilization of these kinds of proteins may provide a novel therapeutic strategy.

In an aspect, this disclosure provides a method of treating AMVPK, cGAS, or ΔF508-CFTR mediated diseases, the method including administering one or more AMVPK, cGAS, or CFTR DUBTACs to a subject who has an AMPK, cGAS, or ΔF508-CF TR-mediated disease, the AMIPK, cGAS, or CFTR DUBTACs being bivalent compounds including an AMPK, cGAS, or CFTR ligand conjugated to a de-ubiquitination tag via a linker, which would stabilize AMIPK, cGAS, or CFTR. The AMPK, cGAS, or ΔF508-CFTR -mediated disease can be a disease resulting from AMPK, cGAS, or CFTR destabilization. The AMPK, cGAS, or ΔF508-CFTR-mediated disease can have reduced AMPK, cGAS, or CFTR expression relative to a wild-type tissue of the same species and tissue type.

More specifically, the present disclosure provides a bivalent compound including an AMPK, cGAS, or CFTR ligand conjugated to OTUB1 recruiter via a linker.

In some aspects, the AMPK, cGAS, or CFTR DUBTACs have the form “PI-Linker-OTUB1 Recruiter” as shown below.

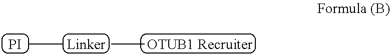

wherein PI (a ligand for a “protein of interest,” i.e., the protein to be subjected to de-ubiquitination) (e.g., a AMPK, cGAS, or CFTR ligand), and OTUB1 Recruiter comprises a de-ubiquitination tag (e.g., a ligand for OTUB1). Exemplary AMPK, cGAS, or CFFTR ligands (PI), exemplary OTUB1 binders (OTUB1 Recruiters), and exemplary linkers (Linker) are illustrated below:

### PI

### AMPK Ligand

In some embodiments, the AMPK ligand can be an AMPK activator or AMPK inhibitor which potently binds to AMPK. In some embodiments, the AMPK ligand comprises Metformin (Xiao et al., 2020), AICAR (Nakamaru et al., 2005), ZMP, AMP, A-769662 (Sanders et al., 2007), 991 (Ngoei et al., 2018), PF-06685249 (Edmonds et al., 2018), PXL770 (Cusi et al., 2021), MK-8722 (Myers et al., 2017), PF-739 (Aledavood et al., 2021), 4-azaindole, RSVA405 (Vingtdeux et al., 2011), COH—SR4 (Figarola and Rahbar, 2013), B10 (Sun et al., 2020), Dorsomorphin (Kim et al., 2011), BAY-3827 (Lemos et al., 2021), GSK-690693 (Rhodes et al., 2008) or a derivative thereof. In some embodiments, the AMPK ligand is a compound disclosed in one or more of WO2009124636, WO2009100130, WO2010036613, WO2011029855, WO2011080277, WO2011032320, WO2013116491, WO2014133008, WO2016008404, WO2016001224, each of which is incorporated by reference in its entirety.

In some embodiments, the AMPK ligand is an AMPK inhibitor. In some embodiments, the AMPK ligand is an ATP-competitive AMPK inhibitor. In some embodiments, the AMPK ligand is an AMPK activator. In some embodiments, the AMPK ligand is an AMPK activator, where the AMPK activator binds to an allosteric binding site. In some embodiments, the AMPK activator binds to an allosteric binding site located at the α/β subunit interface. In some embodiments, the AMPK activator has different AMPK isoform selectivity. In some embodiments, the AMPK activator is a pan-AMPK isoform activator.

In some embodiments, the AMPK ligand comprises the structure of Formula (B-I):

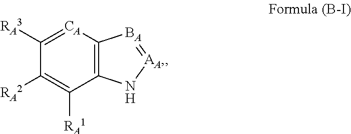

or a pharmaceutically acceptable salt of the compound or the tautomer thereof, wherein
A_{A} is selected from N, CR_{A}⁴, or

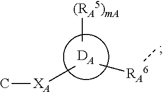

B_{A} is selected from N, CR_{A}⁴, or

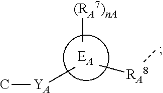

[symbol] denotes the point of attachment to Linker in Formula (B);
C_{A} is selected from N or CR_{A}4; Ring D_{A} and Ring E_{A} are independently selected from null, C₃-C₁₂ cycloalkyl, 3-12-membered heterocyclic, C₆-C₁₀ aryl, and 5-10 membered heteroaryl;
X_{A} and YA are independently selected from a bond, —O—, —S—, —NR_{A}⁹—, —C(O)—, —C(O)O—, —C(O)NR_{A}⁹—, —O—C(O)NR_{A}⁹—, —NR_{A}¹⁰C(O)NR_{A}⁹—, —S(O)—, —S(O)₂—, —S(O)NR_{A}⁹—, —S(O)₂NR_{A}⁹—, C₁-C₆ alkylene, C₁-C₆ haloalkylene, C₁-C₆ heteroalkylene, C₂-C₆ alkenylene, and C₂-C₆ alkynylene;
R_{A}¹, R_{A}², each R_{A}⁴, each R_{A}⁵, and each R_{A}⁷ are independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
R_{A}³ is selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{A}¹ and R_{A}², R_{A}² and R_{A}³, R_{A}³ and R_{A}⁴, two R_{A}⁵, or two R_{A}⁷, together with the atoms to which they are attached, optionally form C₄-C₆ cycloalkyl or 5-7 membered heterocyclic, C₆-C₁₀ aryl, and 5-10 membered heteroaryl;
R_{A}⁶ and R_{A}⁸ are independently selected from a bond, —O—, —NR_{A}³—, —C(O)—, —C(O)O—, —C(O)NR_{A}¹³—, —O—C(O)NR_{A}¹³—, —NR_{A}¹⁴C(O)NR_{A}¹³—, —S(O)—, —S(O)₂—, —S(O)NR_{A}¹³—, —S(O)₂NR_{A}¹³—, C₁-C₆ alkylene, C₁-C₆ haloalkylene, C₁-C₆ heteroalkylene, C₂-C₆ alkenylene, C₂-C₆ alkynylene, C₆-C₁₀ arylene, and 5-10 membered heteroarylene;
each R_{A}⁹, each R_{A}¹⁰, each R_{A}¹¹, each R_{A}¹², each R_{A}¹³, and each R_{A}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl; or
R_{A}¹¹ and R_{A}¹² together with the atoms to which they are attached, optionally form 3-12 membered heterocyclic; and
m_{A} and n_{A} are independently selected from 0, 1, 2, 3, and 4.

In some embodiments, A_{A} is N; and B_{A} is

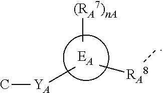

In some embodiments, A_{A} is CR_{A}⁴; and B_{A} is

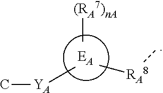

In some embodiments, A_{A} is

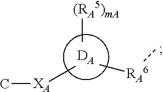

and B_{A} is N. In some embodiments, A_{A} is

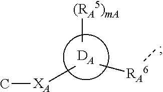

and B_{A} is CR_{A}⁴.

In some embodiments, the AMPK ligand comprises the structure of Formulae (B-I-a) and Formulae (B-I-b):

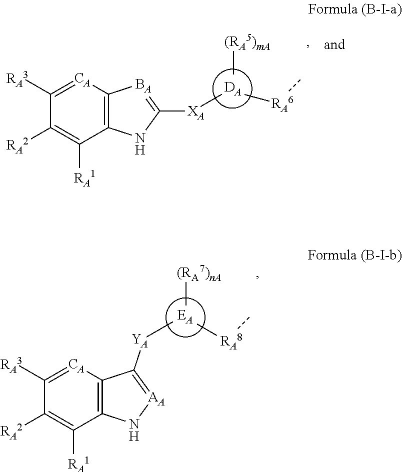

wherein
A_{A} and are B_{A} independently selected from N, CR_{A}⁴.

In some embodiments, X_{A} is selected from —O—, —S—, —NR_{A}⁹—, —C(O)—, C₁-C₆ alkylene, C₁-C₆haloalkylene, C₁-C₆ heteroalkylene, C₆-C₁₀ arylene, and 5-10 membered heteroarylene. In some embodiments, X_{A} is selected from —O—, —S—, —NR_{A}⁹—, —C(O)—, methylene, and halomethylene, In some embodiments, XA is selected from —O—, —S—, —NH—, —C(O)—, CH₂, and CF₂, In some embodiments, X_{A} is —O—.

In some embodiments, Y_{A} is selected from a bond, —C(O)—, —C(O)O—, —C(O)NR_{A}⁹—, —S(O)—, —S(O)₂—, —S(O)NR_{A}⁹—, —S(O)₂NR_{A}⁹—. In some embodiments, Y_{A} is selected from a bond, —C(O)—, —C(O)NR_{A}⁹—, and —S(O)₂NR_{A}⁹—. In some embodiments, Y_{A} is —C(O)NR_{A}⁹—.

In some embodiments, the AMPK ligand comprise the structure of Formulae (B-I-a1), (B-I-b1), and (B-I-b2):

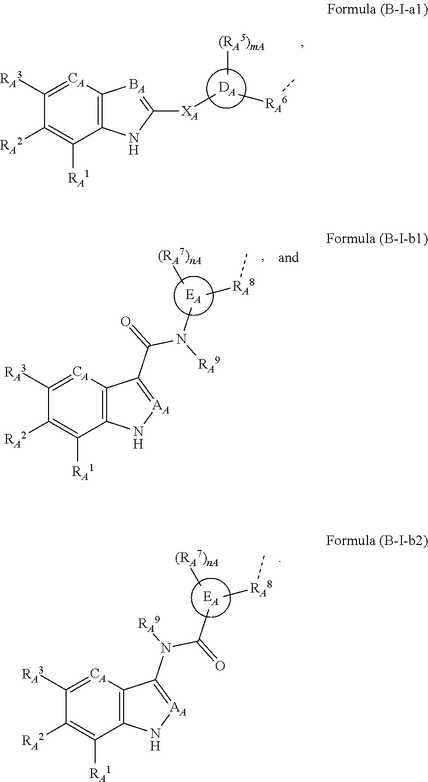

In some embodiments, A_{A} is N. In some embodiments, A_{A} is CR_{A}⁴. In some embodiments, B_{A} is N. In some embodiments, B_{A} is CR_{A}⁴. In some embodiments, C_{A} is N. In some embodiments, C_{A} is CR_{A}⁴.

In some embodiments, Ring D_{A} is selected from null, C₃-C₁₂ cycloalkyl, 3-12-membered heterocyclic, C₆-C₁₀ aryl, and 5-10 membered heteroaryl. In some embodiments, Ring D_{A} is C₃-C₁₂ cycloalkyl. In some embodiments, Ring D_{A} is selected from cyclobutyl, cyclopropyl, cyclobutyl, cyclopentyl, cyclohexyl, and cycloheptyl. In some embodiments, Ring D_{A} is 3-12-membered heterocyclic. In some embodiments, Ring D_{A} is selected from tetrahydrofuryl, hexahydrofuro[3,2-b]furyl, azetidinyl, pyrrolidinyl, piperidinyl, and piperazinyl. In some embodiments, Ring D_{A} is C₆-C₁₀ aryl. In some embodiments, Ring D_{A} is phenyl. In some embodiments, Ring D_{A} is 5-10 membered heteroaryl. In some embodiments, Ring D_{A} is selected from thiophenyl, benzothiophenyl, tetrahydrobenzothiophenyl, thiazolyl, imidazolyl, furanyl, pyrrolyl, pyrazolyl, pyridinyl, pyrimidinyl, pyridazinyl, pyrazinyl, benzofuranyl, indolyl, and indazolyl.

In some embodiments, Ring E_{A} selected from null, C₃-C₁₂ cycloalkyl, 3-12-membered heterocyclic, C₆-C₁₀ aryl, and 5-10 membered heteroaryl. In some embodiments, Ring E_{A} selected from null, C₆-C₁₀ aryl, and 5-10 membered heteroaryl. In some embodiments, Ring E_{A} selected from null. In some embodiments, Ring E_{A} is C₆-C₁₀ aryl. In some embodiments, Ring E_{A} is phenyl.. In some embodiments, Ring E_{A} is 5-10 membered heteroaryl. In some embodiments, Ring EA is selected from thiophenyl, benzothiophenyl, tetrahydrobenzothiophenyl, thiazolyl, imidazolyl, furanyl, pyrrolyl, pyrazolyl, pyridinyl, pyrimidinyl, pyridazinyl, pyrazinyl, benzofuranyl, indolyl, and indazolyl.

In some embodiments, the AMPK ligand comprises the structure of Formulae (B-I-a2), (B-I-a3), (B-I-a4), (B-I-b3), and (B-I-b4):

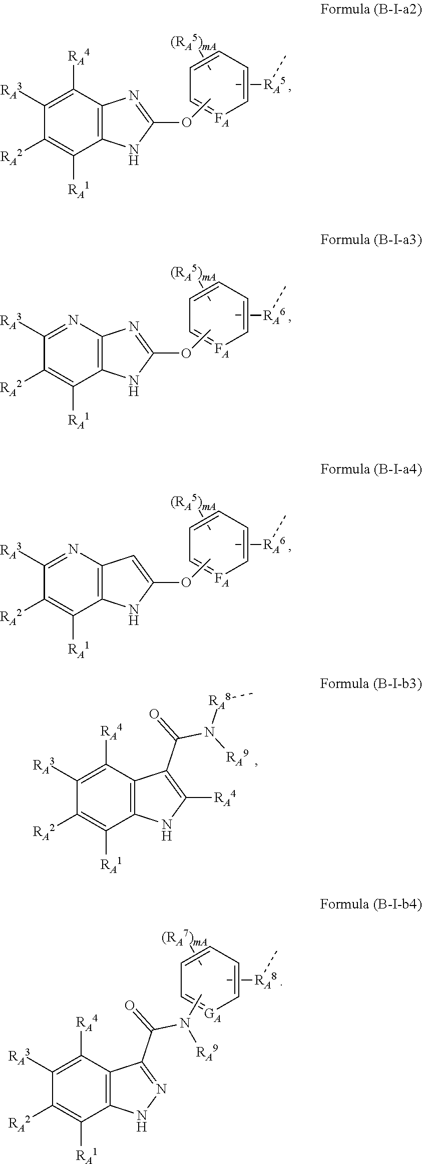

wherein
F_{A} is selected from N or CR_{A}⁵; and
G_{A} is selected from N or CR_{A}⁷.

In some embodiments, R_{A}³ is selected from C₃-C₁₂ cycloalkyl, 3-12 membered heterocyclic, C₆-C₁₀ aryl, and 5-10 membered heteroaryl. In some embodiments, R_{A}³ is selected from C₆-C₁₀ aryl and 5-10 membered heteroaryl.

In some embodiments, the AMPK ligand comprises the structure of Formulae (B-I-a5), (B-I-a6), (B-I-a7), (B-I-b5), and (B-I-b6):

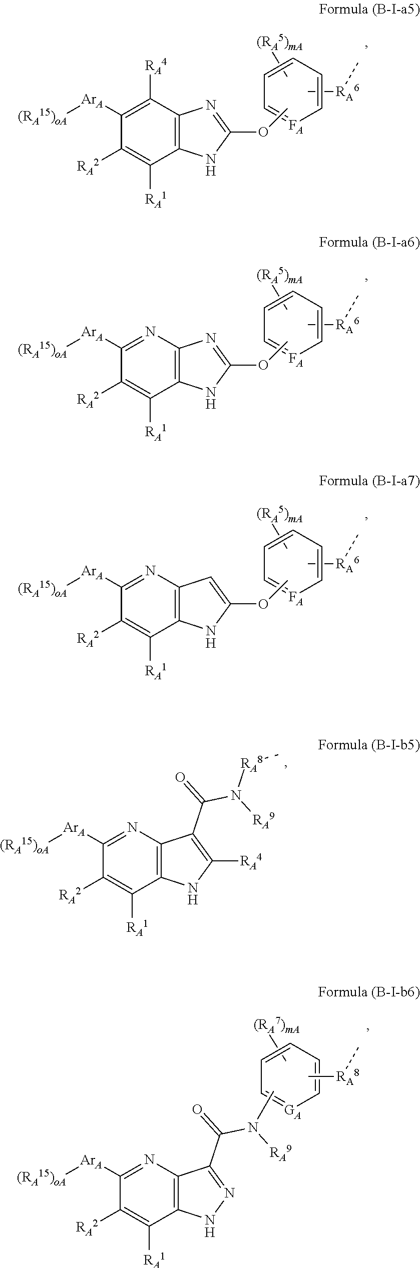

wherein
Ar_{A} is selected from C₆-C₁₀ aryl, or 5-10 membered heteroaryl;
each R_{A}¹⁵ is independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², —N═S(O)R_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl and 5-10 membered heteroaryl, or
two of R_{A}¹⁵, together with the atoms to which they are attached, optionally form C₃-C₁₂ cycloalkyl, 3-12 membered heterocyclic, C₆-C₁₀ aryl or 5-10 membered heteroaryl; and
o_{A} is selected from 0, 1, 2, 3, 4, 5, or 6.

In some embodiments, Ar_{A} is selected from C₆-C₁₀ aryl and 5-10 membered heteroaryl. In some embodiments, Ar_{A} is selected from phenyl, naphthalene, indane, 5,6,7,8-tetrahydronaphthalene, biphenyl, pyrazole, pyridine, pyrazine, pyrimidine, thiazole, thiophene, benzoimidazole, quinoline, isoquinoline, indole, indazole, carbazole, benzotriazole, benzofuran, benzothiazole, benzo[b]thiophene, benzo[d]isooxazole, 3,4-dihydro-2H-benzo[1,4]oxazine, benzo[1,3]dioxole, benzo[1,4]dioxane, 1H-pyrrolo[2,3-b]pyridine, [1,2,4]triazolo[4,3-a]pyridine, 3,4 dihydropyrido[3,2-b][1,4]oxazine, 3,4-dihydro-2H-1,4-benzoxazine, 2,3-dihydro-1H-indole, 2,3-dihydro-1H-isoindole, 2,3-dihydrobenzoimidazole, 1,2-dihydroquinoline, 1,2,3,4-tetrahydroisoqumoline, 1,2,3,4-tetrahydrocyclopenta[b]indole, 1,2,3,4-tetrahydroquinoxaHne, 1,2,3,6-tetrahydropyridine. In some embodiments, Ar_{A} is selected from phenyl, pyridine, biphenyl, and indole. In some embodiments, Ar_{A} is indole.

In some embodiments, R_{A}⁶ is selected from a bond, —O—, —NR_{A}¹³—, —C(O)—, —C(O)O—, —C(O)NR_{A}¹³—, —S(O)₂NR_{A}¹³—, C₁-C₆ alkylene, C₁-C₆ haloalkylene, and C₁-C₆ heteroalkylene. In some embodiments, R_{A}⁶ is selected from —C(O)—, —C(O)O—, and —C(O)NR_{A}¹³—. In some embodiments, R_{A}⁶ is —C(O)NR_{A}¹³—. In some embodiments, R_{A}⁶ is

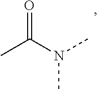

where the N atom of the amide attaches two Linker-OTUB1 Recruiter moieties.

In some embodiments, R_{A}⁸ is selected from a bond, —O—, —NR_{A}¹³—, —C(O)—, —C(O)O—, —C(O)NR_{A}¹³—, —S(O)₂NR_{A}¹³—, C₁-C₆ alkylene, C₁-C₆ haloalkylene, and C₁-C₆ heteroalkylene.

In some embodiments, R_{A1} is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}¹ is selected from H and CH₃. In some embodiments, R_{A1} is H. In some embodiments, R_{A1} is CH₃.

In some embodiments, R_{A}² is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}² is selected from halogen. In some embodiments, R_{A}² selected from F or C₁. In some embodiments, R_{A}² is F. In some embodiments, R_{A}² is Cl.

In some embodiments, each R_{A}⁴ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}⁴ is H.

In some embodiments, each R_{A}⁵ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, each R_{A}⁵ is selected from H, halogen, C₁-C₆ alkyl, C₁-C₆ haloalkyl, C₁-C₆ heteroalkyl, C₃-C₁₂ cycloalkyl. In some embodiments, R_{A}⁵ is H, CH₃, CH₂CH₃, and CH₂OH.

In some embodiments, each R_{A}⁷ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, each R_{A}⁷ is selected from H, halogen, C₁-C₆ alkyl, C₁-C₆ haloalkyl, C₁-C₆ heteroalkyl, C₃-C₁₂ cycloalkyl. In some embodiments, R_{A}⁷ is H, CH₃, and CH₂CH₃.

In some embodiments, each R_{A}⁹ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}⁹ is independently selected from H, C₁-C₆alkyl, C₁-C₆ haloalkyl, C₁-C₆ heteroalkyl, C₃-C₁₂ cycloalkyl, and 3-12 membered heterocyclic. In some embodiments, R_{A}⁹ is H.

In some embodiments, each R_{A}¹⁰ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹⁰ is independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}¹⁰ is H.

In some embodiments, each R_{A}¹¹ and each R_{A}¹² are independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹¹ and each R_{A}¹² are independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}¹¹ is selected from H and CH₃. In some embodiments, R_{A}¹² is selected from H and CH₃.

In some embodiments, R_{A}¹¹ and R_{A}¹² together with the atoms to which they are attached, optionally form 3-12 membered heterocyclic.

In some embodiments, each R_{A}³ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹³ is independently selected from H, C₁-C₆ alkyl, C₁-C₆ haloalkyl, C₁-C₆ heteroalkyl, C₃-C₁₂ cycloalkyl, and 3-12 membered heterocyclic.

In some embodiments, each R_{A}¹⁴ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹⁴ is independently selected from H, C₁-C₆ alkyl, C₁-C₆ haloalkyl, C₁-C₆ heteroalkyl, C₃-C₁₂ cycloalkyl, and 3-12 membered heterocyclic.

In some embodiments, each R_{A}¹⁵ is independently selected from each R_{A}¹⁵ is independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl and 5-10 membered heteroaryl.

In some embodiments, each R_{A}¹⁵ is independently selected from each R_{A}¹⁵ is independently selected from H, halogen, cyano, OR_{A}¹¹, NR_{A}¹¹R_{A}¹², S(O)₂NR_{A}¹¹R_{A}¹², —N═S(O)R_{A}¹R_{A}¹², C₁-C₆ alkyl, C₁-C₆ haloalkyl, C₁-C₆ heteroalkyl, C₃-C₁₂ cycloalkyl, 3-12 membered heterocyclic, C₆-C₁₀ aryl and 5-10 membered heteroaryl. In some embodiments, each R_{A}¹⁵ is independently selected from each R_{A}¹⁵ is independently selected from H, CN, CH₃, cyclopropyl, hydroxymethyl cyclopropyl, OCH₃, dimethyl amino, —N═S(O)(CH₃)₂, and -Ph-N═S(O)(CH₃)₂.

In some embodiments, m_{A} is selected from 0, 1, 2, 3, and 4. In some embodiments, m_{A} is selected from 0, 1, and 2. In some embodiments, n_{A} is selected from 0, 1, 2, 3, and 4. In some embodiments, n_{A} is selected from 0, 1, and 2. In some embodiments, o_{A} is selected from 0, 1, 2, 3, 4, and 5.

In some embodiments, the AMPK ligand comprise the structure of Formulae (B-I), (B-I-a), (B-I-a1), (B-I-a2) and (B-I-a5).

In some embodiments, the AMPK ligand comprise a derivative of following compounds:

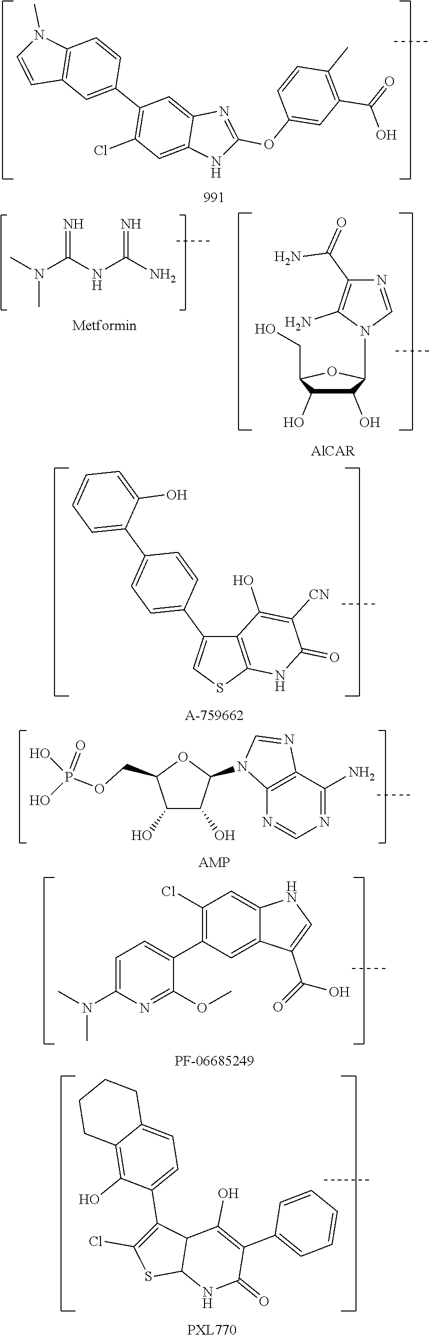

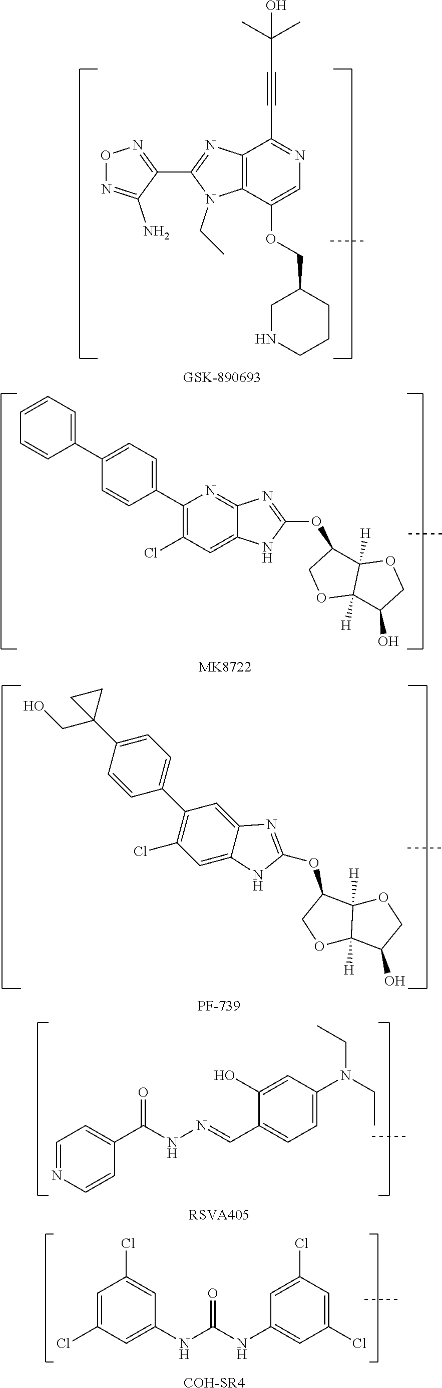

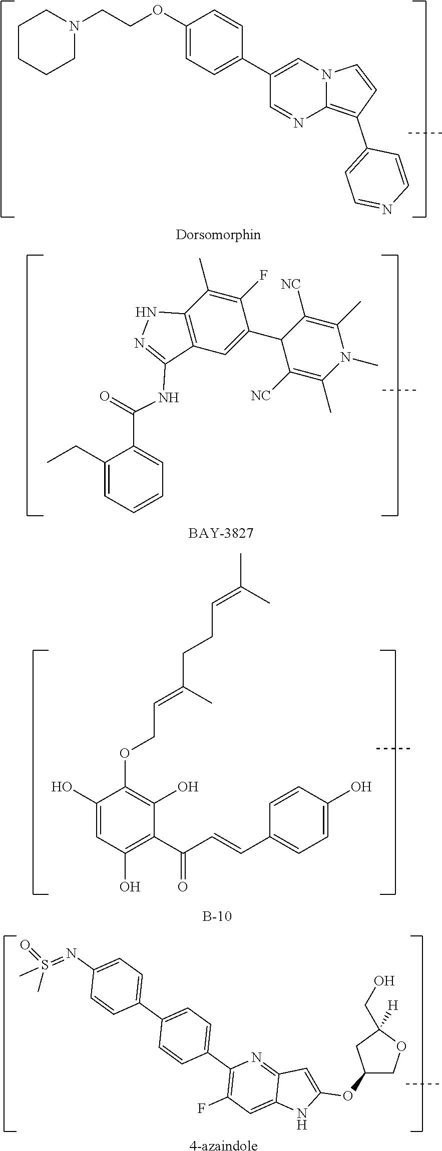

Wherein, [symbol] denotes the point of attachment to Linker.

cGAS Ligand

In some embodiments, the cGAS ligand is seleted from a cGAS activator or cGAS inhibitor which potently binds with cGAS. In some embodiments, the cGAS ligand is cGAS activator. In some embodiments, the cGAS ligand is cGAS inhibitor. In some embodiments, the cGAS ligand comprises hydroxychloroquinine (HCQ), Quinacrine (QC), X6, RU114757, RU191752, RU100840, RU.365, RU.521, J001, G001, G108, G150, G140, CPD-25, aspirin, CPD-C, PF-06928215(Zhao et al., 2022). In some embodiments, the cGAS ligand is a compound disclosed in one or more of U.S. 62/318,435, U.S. 62/355,403, U.S. 2018/0230115, WO2019241787, WO2019153002, WO2022140387, WO2023004437, each of which is incorporated herein by reference in its entirety.

In some embodiments, the cGAS ligand comprise the structure of Formula (C-I):

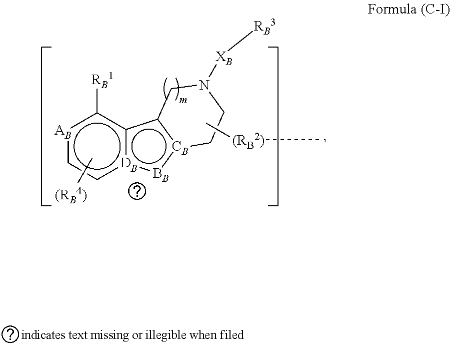

or a pharmaceutically acceptable salt of the compound or the tautomer thereof,
wherein
[symbol] denotes the point of attachment to Linker in Formula (B);
[symbol] connects to either R_{B}^{1b} or R_{B}^{3b}, with the proviso that when [symbol] connects to R_{B}^{1b}, R_{B}³ is R_{B}^{3a}; and when [symbol] connects to R_{B}^{3b}, R_{B}¹ is R_{B}^{1a};
A_{B} is selected from N and CR_{B}⁴;
B_{B} is selected from N, O, S, CR_{B}⁵ and NR_{B}⁵;
C_{B} is selected from N and C;
D_{B} is selected from N and C;
X_{B} is X_{B}^{a}—X_{B}^{b};
X_{B}^{a} is selected from a bond, —C(O)—, —C(O)O—, —C(O)NR_{B}⁶—, —S(O)—, —S(O)₂—, —S(O)NR_{B}⁶—, —S(O)₂NR_{B}⁶—;
X_{B}^{b} is selected from a bond, C₁-C₆ alkylene, C₁-C₆ haloalkylene, C₁-C₆ heteroalkylene, C₃-C₁₂ cycloalkylene, and 3-12 membered heterocyclicene, C₆-C₁₀ arylene, and 5-10 membered heteroarylene;
R_{B}¹ is selected from monovalent group R_{B}^{1a} and bivalent group R_{B}^{1b};
R_{B}^{1a} is selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
R_{B}^{1b} is connected to the Linker, and is selected from a bond, —O—, —NR_{B}⁷—, —C(O)O—, —C(O)NR_{B}⁷—, —S(O)₂NR_{B}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12 membered heterocyclicene, C₆-C₁₀ arylene, and 5-10 membered heteroarylene;
each R_{B}² is independently selected from H, halogen, C₁-C₆ alkyl, C₁-C₆ haloalkyl, C₁-C₆ heteroalkyl, C₃-C₁₂ cycloalkyl, or
two R_{B}², together with the atoms to which they are attached, optionally form C₃-C₁₂ cycloalkyl or 3-12 membered heterocyclic;
R_{B}³ is selected from monovalent group R_{B}^{3a} and bivalent group R_{B}^{3b};
R_{B}^{3a} is selected from H, halogen, OR_{B}⁹, NR_{B}⁹R_{B}¹⁰, C(O)OR_{B}⁹, C(O)NR_{B}⁹R_{B}¹⁰, S(O)₂NR_{B}⁹R_{B}¹⁰, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
R_{B}^{3b} is connected to the Linker, and is selected from a bond, —O—, —NR_{B}⁹—, —C(O)O—, —C(O)NR_{B}⁹—, —S(O)₂NR_{B}⁹—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, and 3-12 membered heterocyclicene;
each R_{B}⁴ is independently selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₃ alkyl, C₁-C₃ haloalkyl, C₁-C₃ heteroalkyl, C₂-C₄ alkenyl, C₂-C₄ alkynyl, C₃-C₆ cycloalkyl, and 3-6 membered heterocyclic, or
two R_{B}⁴, together with the atoms to which they are attached, optionally form partially unsaturated C₃-C₁₂ cycloalkyl, partially unsaturated 3-12 membered heterocyclic, C₆-C₁₀ arylene, or 5-10 membered heteroarylene;
R_{B}⁵ is selected from H, halogen, C₁-C₃ alkyl, C₁-C₃ haloalkyl, C₁-C₃ heteroalkyl, C₃-C₆ cycloalkyl, and 3-6 membered heterocyclic;
R_{B}⁶, each R_{B}⁷, each R_{B}⁸, each R_{B}⁹, and each R_{B}¹⁰, are independently selected from H, C₁-C₆ alkyl, C₁-C₆ haloalkyl, C₁-C₆ heteroalkyl, C₂-C₆alkenyl, C₂-C₆ alkynyl, C₃-C₁₂ cycloalkyl, and 3-12 membered heterocyclic, or
R_{B}⁷ and R_{B}⁸, and R_{B}⁹ and R_{B}¹⁰ together with the atoms to which they are attached, optionally form 3-12 membered heterocyclic;
m_{B} is selected from 1 and 2;
n_{B} is selected from 0, 1, 2 and 3; and
o_{B} is selected from 0, 1, 2, 3, 4, 5 and 6.

In some embodiments, [symbol] connects to R_{B}^{1b}, R_{B}³ is R_{B}^{3a}. In some embodiments, [symbol] connects to R_{B}^{3b}, R_{B}¹ is R_{B}^{1a}.

In some embodiments, the cGAS ligand comprises the structures of Formulae (C-I-a) and (C-I-b):

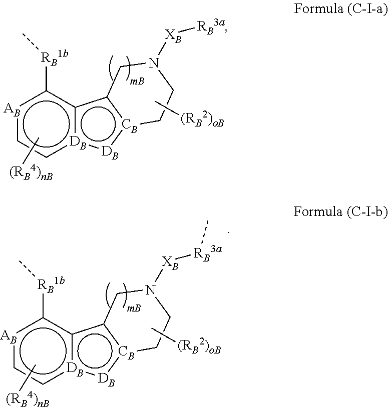

In some embodiments, A_{B} is CR_{B}⁴. In some embodiments, B_{B} is NR_{B}⁵. In some embodiments, C_{B} is C. In some embodiments, D_{B} is C.

In some embodiments, the cGAS ligand comprises the structures of Formulae (C-I-a1) and (C-I-b1):

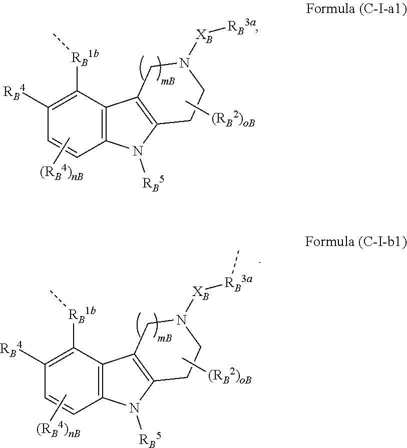

In some embodiments, R_{B}^{1a} is selected from H, halogen, C₃-C₁₂ cycloalkyl, 3-12 membered heterocyclic, C₆-C₁₀ aryl, and 5-10 membered heteroaryl. In some embodiments, R_{B}^{1a} is selected from C₃-C₁₂ cycloalkyl, 3-12 membered heterocyclic, C₆-C₁₀ aryl, and 5-10 membered heteroaryl. In some embodiments, R_{B}^{1a} is selected from C₆-C₁₀ aryl, and 5-10 membered heteroaryl. In some embodiments, R_{B}^{1a} is 5-10 membered heteroaryl. In some embodiments, R_{B}^{1a} is selected from furyl, thiophenyl, pyrrolyl, pyrazolyl, oxazolyl, oxadiazolyl, thiazolyl, isoxazolyl, isothiazolyl, imidazolyl, triazolyl, pyridinyl, pyrimidinyl, pyrazinyl, and pyridazinyl. In some embodiments, R_{B}^{1a} is pyrazolyl. In some embodiments, R_{B}^{1a} is =pyridinyl.

In some embodiments, R_{B}^{1b} is selected from C₃-C₁₂ cycloalkylene, 3-12 membered heterocyclicene, C₆-C₁₀ arylene, and 5-10 membered heteroarylene. In some embodiments, R_{B}^{1b} is selected from C₆-C₁₀ arylene, and 5-10 membered heteroarylene.

In some embodiments, the cGAS ligand comprises the structures of Formulae (C-I-a2) and (C-I-b2):

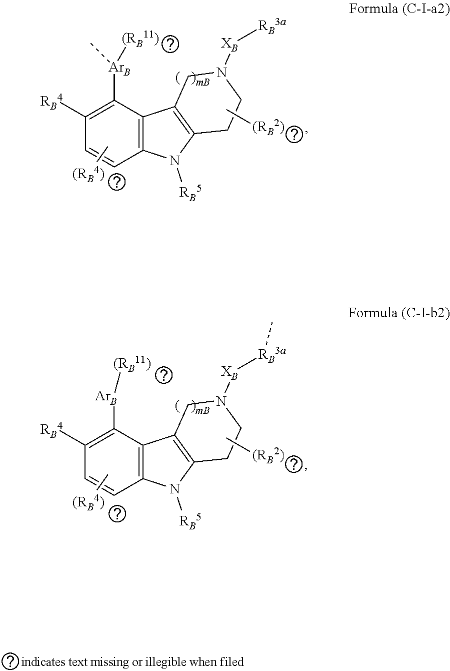

wherein
Ar_{B} is selected from C₆-C₁₀ aryl, and 5-10 membered heteroaryl;
each R_{B}¹¹ is independently selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₆ alkyl, C₁-C₆ haloalkyl, C₁-C₆ heteroalkyl, C₂-C₆ alkenyl, C₂—C₆ alkynyl, C₃-C₁₂ cycloalkyl, 3-12 membered heterocyclic, C₆-C₁₀ aryl, and 5-10 membered heteroaryl; or
two R_{B}¹¹, together with the atoms to which they are attached, optionally form partially unsaturated C₃-C₁₂ cycloalkyl, partially unsaturated 3-12 membered heterocyclic, C₆-C₁₀ aryl, or 5-10 membered heteroaryl;
p_{B} is selected from 0, 1, 2, 3, 4, and 5; and
p_{B-1} is selected from 0, 1, 2, 3, and 4.

In some embodiments, X_{Ba} is selected from —C(O)—, —S(O)—, and —S(O)₂—. In some embodiments, X_{B}^{a} is —C(O)—. In some embodiments, X_{B}^{b} is selected from C₁-C₆ alkylene, C₁-C₆ haloalkylene, C₁-C₆ heteroalkylene, C₃-C₁₂ cycloalkylene, and 3-12 membered heterocyclicene.

In some embodiments, the cGAS ligand comprises the structures of Formulae (C-I-a3) and (C-I-b3):

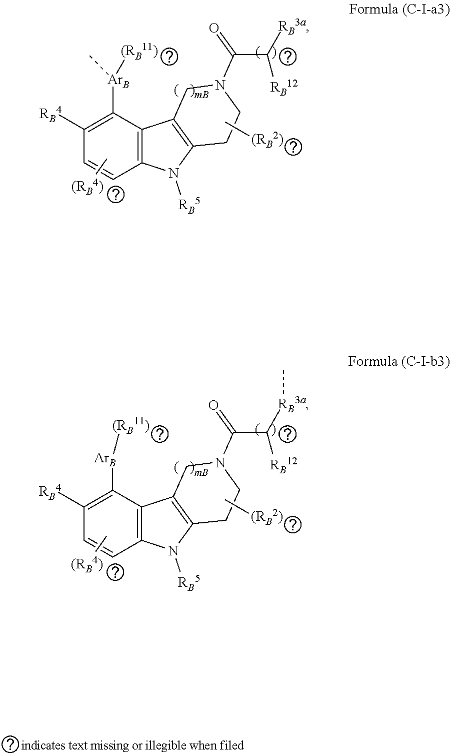

wherein
each R_{B}¹² is independently selected from H, halogen, OR_{B}⁷, NR_{B}⁷R_{B}⁸, C₁-C₆ alkyl, C₁-C₆ haloalkyl, C₁-C₆ heteroalkyl, C₃-C₁₂ cycloalkyl, and 3-12 membered heterocyclic; or
two R_{B}¹², R_{B}¹² and R_{B}^{3a}, and R_{B}¹² and R_{B}^{3b}, together with the atoms to which they are attached, optionally form C₃-C₁₂ cycloalkyl, 3-12 membered heterocyclic; and
q_{B} is slected from 0, 1, 2, 3, 4, and 5.

In some embodiments, Ar_{B} is 5-10 membered heteroaryl. In some embodiments, Ar_{B} is selected from furyl, thiophenyl, pyrrolyl, pyrazolyl, oxazolyl, oxadiazolyl, thiazolyl, isoxazolyl, isothiazolyl, imidazolyl, triazolyl, pyridinyl, pyrimidinyl, pyrazinyl, and pyridazinyl. In some embodiments, Ar_{B} is pyrazolyl. In some embodiments, Ar_{B} is pyridinyl.

In some embodiments, the cGAS ligand comprises the structures of Formulae (C-I-a4) and (C-I-b4):

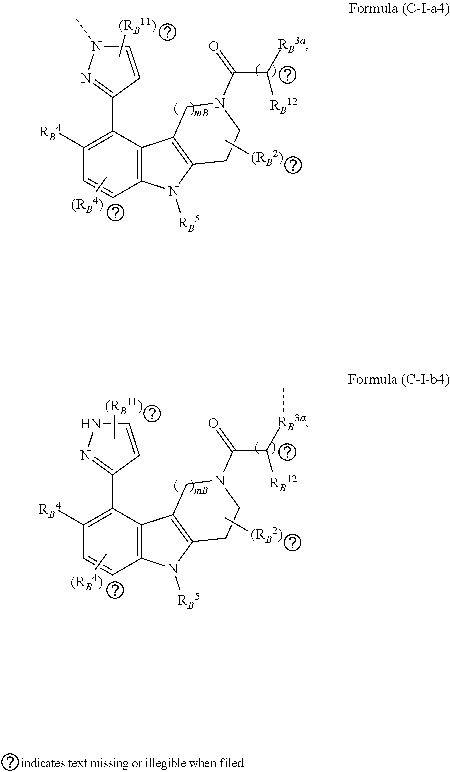

In some embodiments, each R_{B}² is independently selected from H, C₁-C₃ alkyl, C₁-C₃ haloalkyl, C₁-C₃ heteroalkyl, C₃-C₆ cycloalkyl. In some embodiments, R_{B}² is H. In some embodiments, R_{B}² is C₁-C₃ alkyl. In some embodiments, R_{B}² is CH₃. In some embodiments, two R_{B}², together with the atoms to which they are attached, optionally form C₃-C₆ cycloalkyl or 3-6 membered heterocyclic. In some embodiments, two R_{B}², together with the atoms to which they are attached, optionally form C₃-C₅ cycloalkyl.

In some embodiments, R_{B}^{3a} is selected from H, halogen, OR_{B}⁹, NR_{B}⁹R_{B}¹⁰, C₁-C₆ alkyl, C₁-C₆ haloalkyl, C₁-C₆ heteroalkyl, C₃-C₁₂ cycloalkyl, and 3-12 membered heterocyclic. In some embodiments, R_{B}^{3a} is selected from H, OR_{B}⁹ and NR_{B}⁹R_{B}¹⁰. In some embodiments, R_{B}^{3a} is selected from H, OH, OCH₃, OCH₂CH₂OCH₃, NH₂, NHCH₃, and NHC(O)CH₂NH₂. In some embodiments, R_{B}³a is OH.

In some embodiments, R_{B}^{3b} is selected from a bond, —O—, —NR_{B}⁹—, C₁-C₆ alkylene, C₁-C₆ haloalkylene, C₁-C₆ heteroalkylene, C₃-C₁₂ cycloalkylene, and 3-12 membered heterocyclicene. In some embodiments, R_{B}^{3b} is selected from a bond, —O—, —NR_{B}⁹—. In some embodiments, R_{B}^{3b} is selected from a bond, —O—, —NH—, and —N(CH₃)—. In some embodiments, R_{B}^{3b} is —O—.

In some embodiments, each R_{B}⁴ is independently selected from H, halogen, cyano, OR_{B}⁷, NR_{B}⁷R_{B}⁸, C₁-C₃ alkyl, C₁-C₃ haloalkyl, C₁-C₃ heteroalkyl, C₂-C₄ alkenyl, C₂-C₄ alkynyl, C₃-C₆ cycloalkyl, and 3-6 membered heterocyclic. In some embodiments, each R_{B}⁴ is independently selected from H. In some embodiments, each R_{B}⁴ is independently selected from cyano. In some embodiments, each R_{B}⁴ is independently selected from halogen. In some embodiments, each R_{B}⁴ is independently selected from F. In some embodiments, each R_{B}⁴ is independently selected from Cl. In some embodiments, each R_{B}⁴ is independently selected from methyl, ethyl, propyl, isopropyl, cyclopropyl, trifluoromethyl, difluoromethyl, methoxy, ethoxy, cyclopropoxy, and azetidinyl. In some embodiments, each R_{B}⁴ is independently selected from H. In some embodiments, each R_{B}⁴ is independently selected from cyano. In some embodiments, each R_{B}⁴ is independently selected from halogen. In some embodiments, each R_{B}⁴ is independently selected from F. In some embodiments, each R_{B}⁴ is independently selected from Cl. In some embodiments, each R_{B}⁴ is independently selected from Br.

In some embodiments, two R_{B}⁴, together with the atoms to which they are attached, optionally form partially unsaturated C₃-C₆ cycloalkyl, partially unsaturated 3-6 membered heterocyclic, C₆-C₁₀ aryl, or 5-10 membered heteroaryl.

In some embodiments, R_{B}⁵ is selected from H, C₁-C₃ alkyl, C₁-C₃ haloalkyl, C₁-C₃ heteroalkyl, C₃-C₆ cycloalkyl, and 3-6 membered heterocyclic. In some embodiments, R_{B}⁵ is selected from H and C₁-C₃ alkyl. In some embodiments, R_{B}⁵ is H. In some embodiments, R_{B}⁵ is CH₃.

In some embodiments, R_{B}⁶, each R_{B}⁷, each R_{B}⁸, each R_{B}⁹, and each R_{B}¹⁰, are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic. In some embodiments, R_{B}⁶, each R_{B}⁷, each R_{B}⁸, each R_{B}⁹, and each R_{B}¹⁰, are independently selected from H, methyl, ethyl, propyl, isopropyl, cyclopropyl, trifluoromethyl, and difluoromethyl. In some embodiments, R_{B}⁶, each R_{B}⁷, each RBS, each R_{B}⁹, and each R_{B}¹⁰, are H. In some embodiments, R_{B}⁶, each R_{B}⁷, each R_{B}, each R_{B}⁹, and each R_{B}¹⁰, are methyl.

In some embodiments, R_{B}⁷ and R_{B}⁸, and R_{B}⁹ and R_{B}¹⁰ together with the atoms to which they are attached, optionally form 3-6 membered heterocyclic.

In some embodiments, each R_{B}¹¹ is independently selected from H, halogen, cyano, OR_{B}⁷, NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic. In some embodiments, R_{B}¹¹ is H. In some embodiments, each R_{B}¹¹ is CH3.

In some embodiments, two R_{B}¹¹, together with the atoms to which they are attached, optionally form partially unsaturated C₃-C₁₂ cycloalkyl, 3-12 membered partially unsaturated heterocyclic, C₆-C₁₀ aryl, or 5-10 membered heteroaryl.

In some embodiments, each R_{B}¹² is independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, each R_{B}¹² is independently selected from H, and C₁-C₆ alkyl. In some embodiments, R_{B}¹² is H.

In some embodiments, two R_{B}¹², R_{B}¹² and R_{B}^{3a}, and R_{B}¹² and R_{B}^{3b}, together with the atoms to which they are attached, optionally form C₃-C₆ cycloalkyl, 3-6 membered heterocyclic.

In some embodiments, m_{B} is 1. In some embodiments, n_{B} is 1. In some embodiments, n_{B} is 2. In some embodiments, o_{B} is 0. In some embodiments, o_{B} is 1. In some embodiments, o_{B} is 2. In some embodiments, p_{B} is 0. In some embodiments, p_{B} is 1. In some embodiments, p_{B} is 2. In some embodiments, p_{B-1} is 0. In some embodiments, p_{B-1} is 1. In some embodiments, q_{B} is 0. In some embodiments, q_{B} is 1. In some embodiments, qn is 2.

In some embodiments, the cGAS ligand comprises the structures of Formulae (C-I), (C-I-a), (C-I-a1), (C-I-a2), (C-I-a3), and (C-I-a4).

In some embodiments, the cGAS ligand may be a derivative of following compounds:

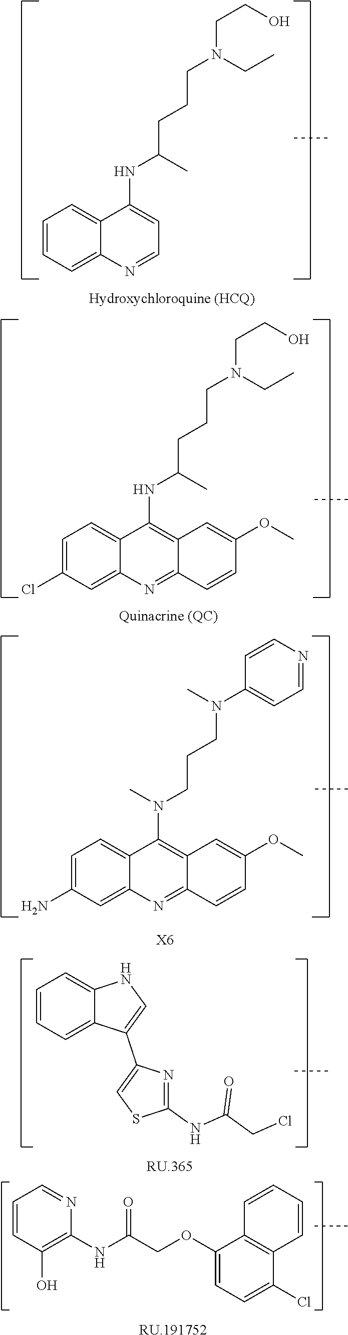

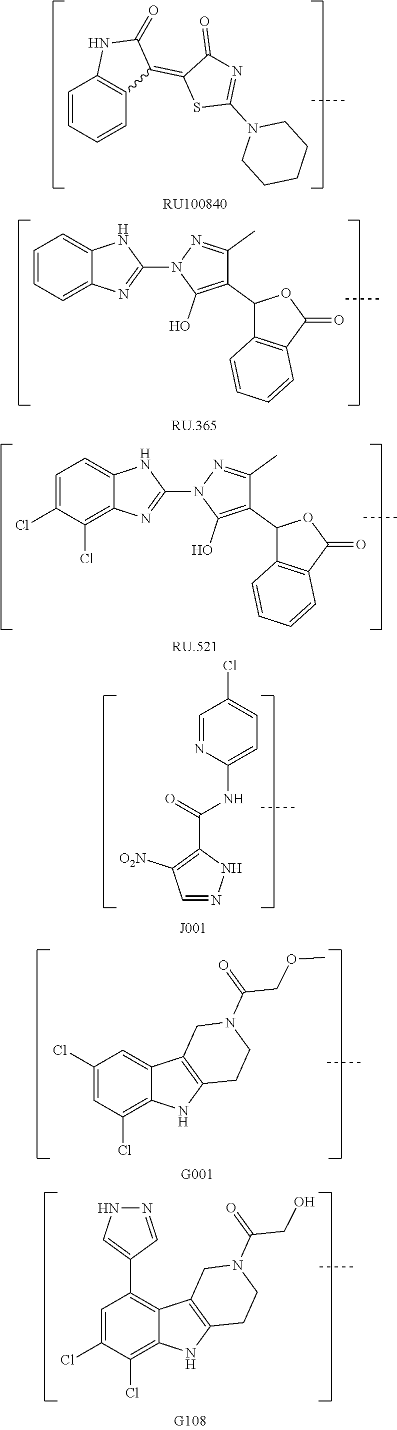

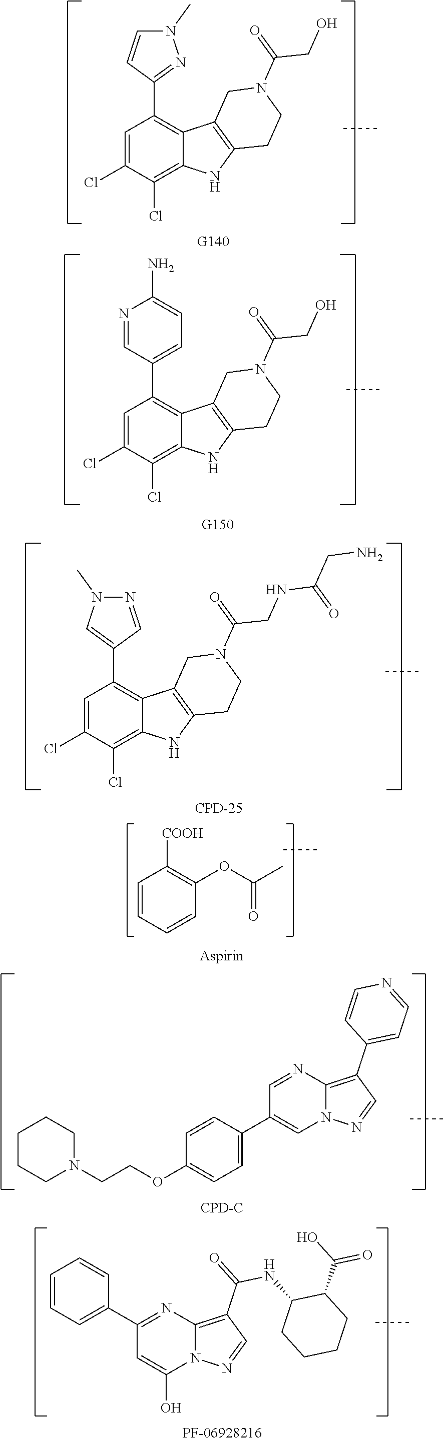

Wherein, [symbol] denotes the point of attachment to Linker.

### CFTR Ligand

In some embodiments, the CFTR ligand can be a CFTR potentiator which potently binds with either wild type CFTR or mutant CFTR. In some embodiments, the CFTR ligand comprises ivacftor, lumacaftor, tezacaftor, elexacafor, or icenticaftor, or derivatives thereof. In some embodiments, the CFTR ligand is a compound disclosed in one or more of U.S. Pat. Nos. 7,999,113; 8,247,436; 8,410,274; WO 2011/133953; and WO2018/037350, each of which is incorporated by reference in its entirety.

In some embodiments, the CFTR ligand comprises the structures of Formula (D-I):

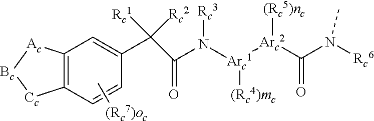

Formula (D-I), or a tautomer thereof, or a pharmaceutically acceptable salt of the compound or the tautomer,
wherein
[symbol] denotes the point of attachment to Linker in Formula (B).
A_{C} and C_{C} are independently selected from O, S, or C(R_{C}⁸)(R_{C}⁹);
B_{C} is C(R_{C}¹⁰)(R_{C}¹¹) or NR_{C}¹²;
Ar_{C}¹ and Ar_{C}² are independently selected from null, C₆-C₁₀ aryl and 5-10 membered heteroaryl;
R_{C}¹ and R_{C}² are independently selected from H, halo, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{C}¹ and R_{C}², together with the atoms to which they are attached, optionally form C₃-C₁₂ cycloalkyl;
R_{C}³ and R_{C}⁶ are independently selected from H, C₁-C₆ alkyl, C₁-C₆ haloalkyl, C₁-C₆ heteroalkyl, C₂-C₆ alkenyl, C₂-C₆ alkynyl, C₃-Cᵤ cycloalkyl, and 3-12 membered heterocyclic;
each R_{C}⁴, each R_{C}⁵, and each R_{C}⁷ are independently selected from H, halogen, cyano, OR_{C}¹³ NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic,
two R_{C}⁴, two R_{C}⁵, or two R_{C}⁷, together with the atoms to which they are attached, optionally form partially unsaturated C₃-C₁₂ cycloalkyl, partially unsaturated 3-12 membered heterocyclic, C₆-C₁₀ aryl and 5-10 membered heteroaryl;
R_{C}⁸, R_{C}⁹, R_{C}¹⁰ and R_{C}¹¹ are independently selected from H, halogen, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₃-C₁₂ cycloalkyl, and 3-12 membered heterocyclic, or
R_{C}⁸ and R_{C}⁹, and R_{C}¹⁰ and R_{C}¹¹, together with the atoms to which they are attached, optionally form C₃-Cᵤ cycloalkyl and 3-12 membered heterocyclic;
R_{C}¹² is selected from H, C₁-C₆ alkyl, C₁-C₆ haloalkyl, C₁-C₆ heteroalkyl, C₃-C₁₂ cycloalkyl, and 3-12 membered heterocyclic; R_{C}¹³ and R_{C}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic, or
R_{C}¹³ and R_{C}¹⁴ together with the atoms to which they are attached, optionally form 3-12 membered heterocyclic;
m_{C} is 0, 1, 2, or 3;
n_{C} is 0, 1, 2, or 3; and
o_{C} is 0, 1, 2, or 3.

In some embodiments, Ar_{C}¹ is 5-10 membered heteroaryl. In some embodiments, Ar_{C}¹ is selected from furyl, thiophenyl, pyrrolyl, pyrazolyl, oxazolyl, oxadiazolyl, thiazolyl, isoxazolyl, isothiazolyl, imidazolyl, triazolyl, pyridinyl, pyrimidinyl, pyrazinyl, and pyridazinyl. In some embodiments, Ar_{B} is pyridinyl. In some embodiments, Ar_{C}² is C₆-C₁₀ aryl. In some embodiments, Ar_{C}² is phenyl.

In some embodiments, the CFTR ligand comprises the structures of Formula (D-I-a):

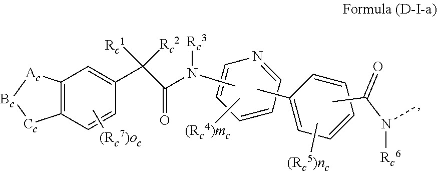

or a tautomer thereof, or a pharmaceutically acceptable salt of the compound or the tautomer.

In some embodiments, the CFTR ligand comprises the structures of Formula (D-I-a1):

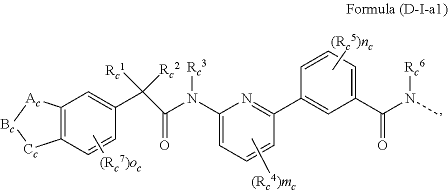

or a tautomer thereof, or a pharmaceutically acceptable salt of the compound or the tautomer.

In some embodiments, A_{C} is O. In some embodiments, B_{C} is C(R_{C}¹⁰)(R_{C}¹¹). In some embodiments, C_{C} is O.

In some embodiments, the CFTR ligand comprises the structures of Formula (D-I-a2):

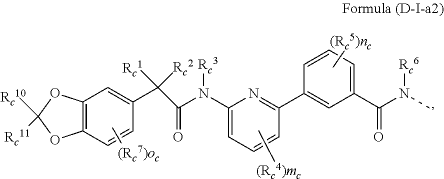

or a tautomer thereof, or a pharmaceutically acceptable salt of the compound or the tautomer.

In some embodiments, R_{C}¹ is selected from H, C₁-C₃ alkyl, C₂-C₃ alkenyl, C₂-C₃ alkynyl, C₃-C₅ cycloalkyl, and 3-5 membered heterocyclic. In some embodiments, R_{C}² is selected from H, C₁-C₃ alkyl, C₂-C₃ alkenyl, C₂-C₃ alkynyl, C₃-C₅ cycloalkyl, and 3-5 membered heterocyclic. In some embodiments, R_{C}¹ and R_{C}², together with the atoms to which they are attached, optionally form C₃-C₆ cycloalkyl. In some embodiments, R_{C}¹ and R_{C}², together with the atoms to which they are attached, optionally form]cyclopropyl.

In some embodiments, R_{C}³ is selected from H, C₁-C₆ alkyl, C₁-C₆ haloalkyl, and C₁-C₆ heteroalkyl. In some embodiments, R_{C}³ is H.

In some embodiments, each R_{C}⁴ is independently selected from H, halo, cyano, OR_{C}¹³, NR_{C}¹³R_{C}¹⁴, C₁-C₆ alkyl, C₂-C₆ alkenyl, C₂-C₆ alkynyl, C₃-C₁₂ cycloalkyl, and 3-12 membered heterocyclic. In some embodiments, each R_{C}⁴ is independetly selected from H and C₁-C₆ alkyl. In some embodiments, each R_{C}⁴ is independently selected from H and CH₃.

In some embodiments, each R_{C}⁵ is independently selected from H, halo, cyano, OR_{C}¹³, NR_{C}¹³R_{C}¹⁴, C₁-C₆ alkyl, C₂-C₆ alkenyl, C₂-C₆ alkynyl, C₃-C₁₂ cycloalkyl, and 3-12 membered heterocyclic. In some embodiments, each R_{C}⁵ is independently selected from H and C₁-C₆ alkyl. In some embodiments, R_{C}⁵ is H.

In some embodiments, R_{C}⁶ is selected from H, C₁-C₆ alkyl, C₁-C₆ haloalkyl, and C₁-C₆ heteroalkyl. In some embodiments, R_{C}⁶ is H. In some embodiments, R_{C}³ is CH₃.

In some embodiments, each R_{C}⁷ is independently selected from H, halogen, cyano, OR_{C}¹, NR_{C}¹³R_{C}¹⁴, C₁-C₆ alkyl, C₂-C₆ alkenyl, C₂-C₆ alkynyl, C₃-C₁₂ cycloalkyl, and 3-12 membered heterocyclic. In some embodiments, each R_{C}⁷ is independently selected from H and C₁-C₆ alkyl.

In some embodiments, R_{C}⁷ is H.

In some embodiments, R_{C}⁸ and R_{C}⁹ are independently selected from H, halogen, C₁-C₆ alkyl, C₁-C₆ haloalkyl, C₁-C₆ heteroalkyl, C₃-C₁₂ cycloalkyl, and 3-12 membered heterocyclic. In some embodiments, R_{C}⁸ and R_{C}⁹ are independently selected from H and C₁-C₆ alkyl,

In some embodiments, R_{C}¹⁰ and R_{C}¹¹ are independently selected from H, halogen, C₁-C₆ alkyl, C₁-C₆ haloalkyl, C₁-C₆ heteroalkyl, C₃-Cᵤ cycloalkyl, and 3-12 membered heterocyclic. In some embodiments, R_{C}¹⁰ and R_{C}¹¹ are independently selected from H, halogen and C₁-C₆ alkyl. In some embodiments. R_{C}¹⁰ and R_{C}¹¹ are independently selected from H, F, Cl, CH₃, and CF₃. In some embodiments, both R_{C}¹⁰ and R_{C}¹¹ are F.

In some embodiments, R_{C}¹² is selected from H, C₁-C₆ alkyl, C₁-C₆ haloalkyl, C₁-C₆ heteroalkyl, C₃-C₁₂ cycloalkyl, and 3-12 membered heterocyclic.

In some embodiments, R_{C}¹³ and R_{C}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{C}¹³ and R_{C}¹⁴ are independently selected from H and C₁-C₆ alkyl. In some embodiments, R_{C}¹³ and R_{C}¹⁴ are independently selected from H and CH₃.

In some embodiments, R_{C}¹³ and R_{C}¹⁴ together with the atoms to which they are attached, optionally form 3-6 membered heterocyclic.

In some embodiments, me is 1. In some embodiments, n_{C} is 0. In some embodiments, o_{C} is 0.

In some embodiments, the CFTR ligand comprises the structures of Formulae (D-I-a3) and (D-I-a4):

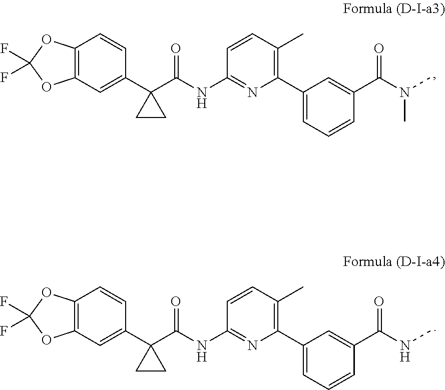

In some embodiments, the CFTR ligand may be a derivative of following compounds:

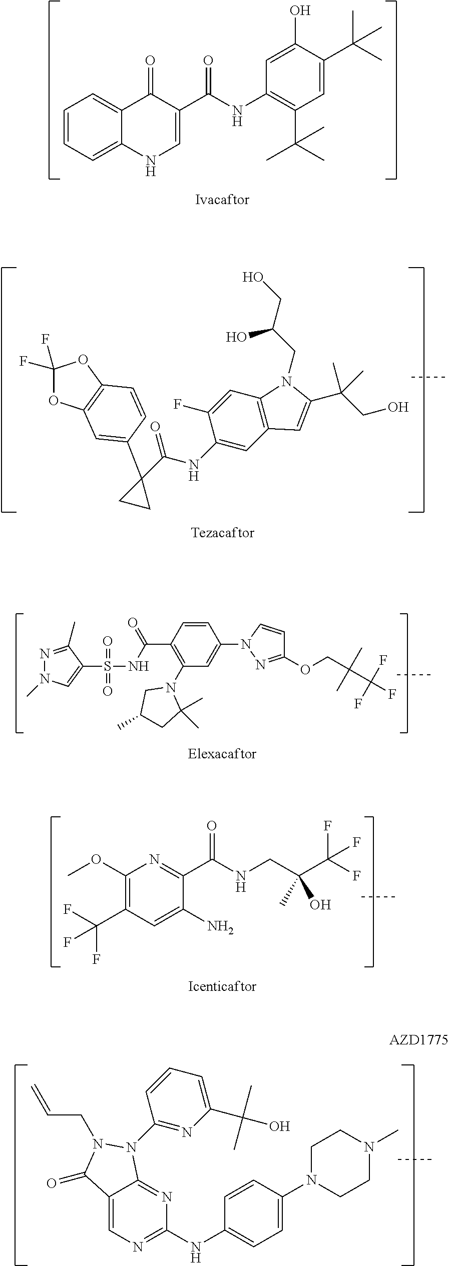

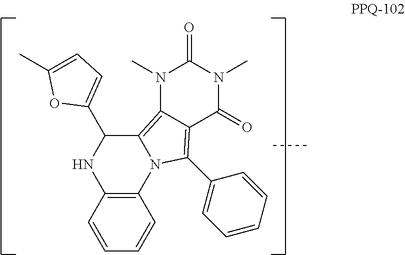

Wherein, [symbol] denotes the point of attachment to Linker.

### Linkers

In all of the above-described compounds, the AMPK, cGAS or CFTR ligand is conjugated to the de-ubiquitination tag through a linker. The linker can include, for example, acyclic or cyclic saturated or unsaturated carbon, ethylene glycol, amide, amino, ether, urea, carbamate, aromatic, heteroaromatic, heterocyclic and/or carbonyl containing groups with different lengths.

In some embodiments, the linker can be a moiety of Formula (E):

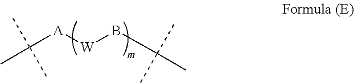

wherein
A, W and B, at each occurrence, are independently selected from null, or bivalent moiety selected from R′-R″, R′COR″, R′CO₂R″, R′C(O)N(R¹)R″, R′C(S)N(R¹)R″, R′OR″, R′OC(O)R″, R′OC(O)OR—, R′OCON(R¹)R′, R′SR″, R′SOR″, R′SO₂R″, R′SO₂N(R¹)R″, RN(R¹)R′, R′NR¹COR″, R′NR¹C(O)OR″, R′NR¹CON(R²)R″, R′NR¹C(S)R″, R′NR¹S(O)R″, R′NR¹S(O)₂R″, and R′NR¹S(O)₂N(R²)R″, wherein
R′ and R″ are independently selected from a bond, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R is selected from optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclic, optionally substituted 4-10 membered heterocyclic, optionally substituted aryl, and optionally substituted heteroaryl;
R′ and R″, R¹ and R², R′ and R¹, R′ and R², R″ and R¹, R′ and R² together with the atom to which they are connected form a 3-20 membered cycloalkyl or 4-20 membered heterocyclic ring; and
m is 0 to 15.

In one embodiment, the linker moiety is of Formula (E-1)

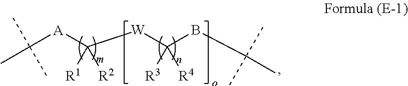

wherein
R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclic, optionally substituted 3-8 membered cycloalkoxy, optionally substituted 3-10 membered carbocyclicamino, optionally substituted 4-8 membered membered heterocyclic, optionally substituted aryl, and optionally substituted heteroaryl, or
R¹ and R², R³ and R⁴ together with the atom to which they are connected form a 3-20 membered cycloalkyl or 4-20 membered heterocyclic ring;
A, W and B, at each occurrence, are independently selected from null, or bivalent moiety selected from R′-R″, R′COR″, R′CO₂R′, R′C(O)N(R⁵)R″, R′C(S)N(R⁵)R″, ROR″, R′OC(O)R″, R′OC(O)OR″, R′OCONR⁵R″, R′SR″, R′SOR″, R′SO₂R″, R′SO₂N(R⁵)R″, R′N(R⁵)R″, R′NR⁵COR″, R′NR⁵C(O)OR′, R′NR⁵CON(R⁶)R″, R′NR⁵C(S)R″, R′NR⁵S(O)R″, R′NR⁵S(O)₂R″, and R′NR⁵S(O)₂N(R⁶)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclic, optionally substituted 4-10 membered heterocyclic, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclic ring;
m is 0 to 15;
n, at each occurrence, is 0 to 15; and
o is 0 to 15.

In another embodiment, the linker moiety is of Formula (E-2)

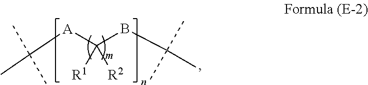

wherein
R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-CsalkylaminoC₁-C₈alkyl, optionally substituted 3-10 membered carbocyclic, optionally substituted 3-8 membered cycloalkoxy, optionally substituted 3-10 membered carbocyclicamino, optionally substituted 4-10 membered heterocyclic, optionally substituted aryl, and optionally substituted heteroaryl, or
R¹ and R² together with the atom to which they are connected form a 3-20 membered cycloalkyl or 4-20 membered heterocyclic ring;
A and B, at each occurrence, are independently selected from null, or bivalent moiety selected from R′—R″, R′COR″, R′CO₂R″, R′C(O)NR³R″, R′C(S)NR³R″, R′OR″, R′OC(O)R″, R′OC(O)OR″, R′OCON(R³)R″, R′SR″, R′SOR″, R′SO₂R″, R′SO₂N(R³)R″, R′N(R³)R″, R′NR³COR″, R′NR³C(O)OR″, R′NR³CON(R⁴)R′, R′NR³C(S)R″, R′NR³S(O)R″, R′NR³S(O)₂R″, and R′NR³S(O)₂N(R⁴)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclic, optionally substituted 4-10 membered heterocyclic, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclic ring;
m, at each occurrence, is 0 to 15; and
n is 0 to 15.

In another embodiment, the linker moiety is of Formula (E-3):

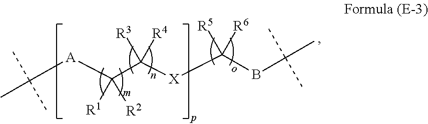

wherein
X is selected from O, NH, and NR⁷;
R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclic, optionally substituted 3-8 membered cycloalkoxy, optionally substituted 4-10 membered heterocyclic, optionally substituted aryl, and optionally substituted heteroaryl;
A and B are independently selected from null, or bivalent moiety selected from R′—R″, R′COR″, R′CO₂R″, R′C(O)N(R⁸)R″, R′C(S)N(R⁸)R″, R′OR″, R′OC(O)R″, R′OC(O)OR″, R′OCON(R⁸)R′, R′SR″, R′SOR″, R′SO₂R″, R′SO₂N(R′)R″, R′N(R⁸)R″, R′NR⁸COR″, R′NR^{B}C(O)OR″, R′NR⁸CON(R⁹)R″, R′NR⁸C(S)R″, R′NR⁸S(O)R″, R′NR⁸S(O)₂R″, and R′NR^{B}S(O)₂N(R⁹)R′, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclic, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclic, optionally substituted 4-10 membered heterocyclic, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclic ring;
m, at each occurrence, is 0 to 15;
n, at each occurrence, is 0 to 15;
o is 0 to 15; and
p is 0 to 15.

In some embodiments, in Formular (E-3), m and n is 0 or 1, and p is 0 to 15;

In some embodiments, in Formular (E-3), X is selected from O and NH;

In some embodiments, in Formular (E-3), R¹, R², R³, R⁴, R⁵, and R⁶, are independently selected from hydrogen, and optionally substituted C₁-C₆ alkyl.

In some embodiments, Formulae E, E-1, E-2, and E-3, the linker moiety comprises a ring selected from the group consisting of a 3 to 13 membered ring, a 3 to 13 membered fused ring, a 3 to 13 membered bridged ring, and a 3 to 13 membered spiro ring.

In some aspects of Formulae E, E-1, E-2, and E-3, the linker moiety comprises a ring selected from the group consisting of Formula F1, F2, F3, F4 and F5:

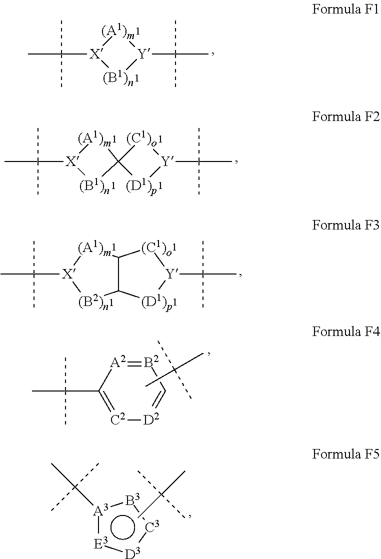

### Wherein

X′ and Y′ are independently selected from N and CR^{b};
A¹, B¹, C¹ and D¹, at each occurrence, are independently selected from null, O, CO, SO, SO₂, NR^{b}, and CR^{b}R^{c};
A², B², C² and D², at each occurrence, are independently selected from N and CR^{b};
A³, B³, C³, D³, and E³ at each occurrence, are independently selected from N, O, S, NR^{b}, and CR^{b};
R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈alkylamino, optionally substituted C₁-C₈ alkylaminoC₁-C₈ alkyl, optionally substituted 3-10 membered carbocyclic, optionally substituted 3-10 membered cycloalkoxy, optionally substituted 3-10 membered carbocyclicamino, optionally substituted 3-8 membered heterocyclic, optionally substituted aryl, and optionally substituted heteroaryl;
R^{b} and R^{b}, or R^{b} and R^{c} together with the atom to which they are connected form a 3-8 membered carbocyclic or 3-8 membered heterocyclic ring; and
m¹, n¹, o¹ and p¹ are independently selected from 0, 1, 2, 3, 4, and 5.

In one embodiment, A, B, and W, at each occurrence, are independently selected from null, optionally substituted —(CH₂)₀₋₈—, optionally substituted —(CH₂)₀₋₈—CO—(CH₂)₀₋₈—, optionally substituted —(CH₂)₀₋₈—NH—(CH₂)₀₋₈—, optionally substituted —(CH₂)₀₋₈—NH—CO—(CH₂)₀₋₈, optionally substituted —(CH₂)₀₋₈—CO—NH—(CH₂)₀₋₈—, optionally substituted —(CH₂)₀₋₃—NH—(CH₂)₀₋₃—CO—NH—(CH₂)₀₋₈—, optionally substituted —(CH₂)₀₋₃—NH—(CH₂)₁₋₃—NH—CO—(CH₂)₀₋₈—, optionally substituted —(CH₂)₀₋₈—CO—NH—(CH₂)₁₋₃—NH—(CH₂)₀₋₃—, optionally substituted —(CH₂)₀₋₈R—(CH₂)₀₋₈—, optionally substituted —(CH₂)₀₋₃—CO—(CH₂)₀₋₃—R—(CH₂)₀₋₃—, —(CH₂)₀₋₈R—(CH₂)₀₋₈—, optionally substituted —(CH₂)₀₋₃—NH—CO—(CH₂)₀₋₃—R^{r}—(CH₂)₀₋₃—, optionally substituted —(CH₂)₀₋₃—NH—(CH₂)₀₋₃—R^{r}—(CH₂)₀₋₃—.

In one embodiment, R^{r} is of Formula F1, F2, F3, F4, or F5.

In one embodiment, R^{r} is selected from

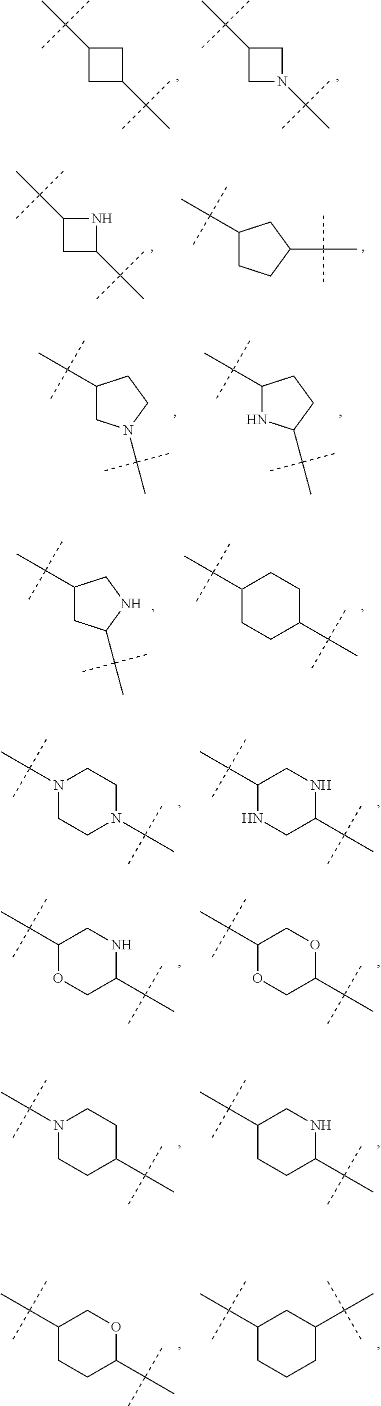

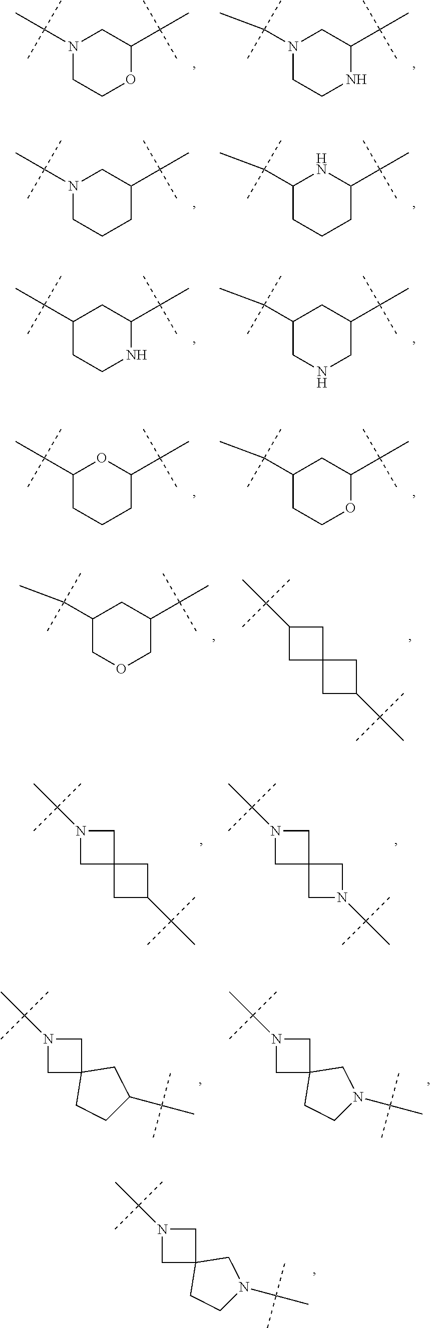

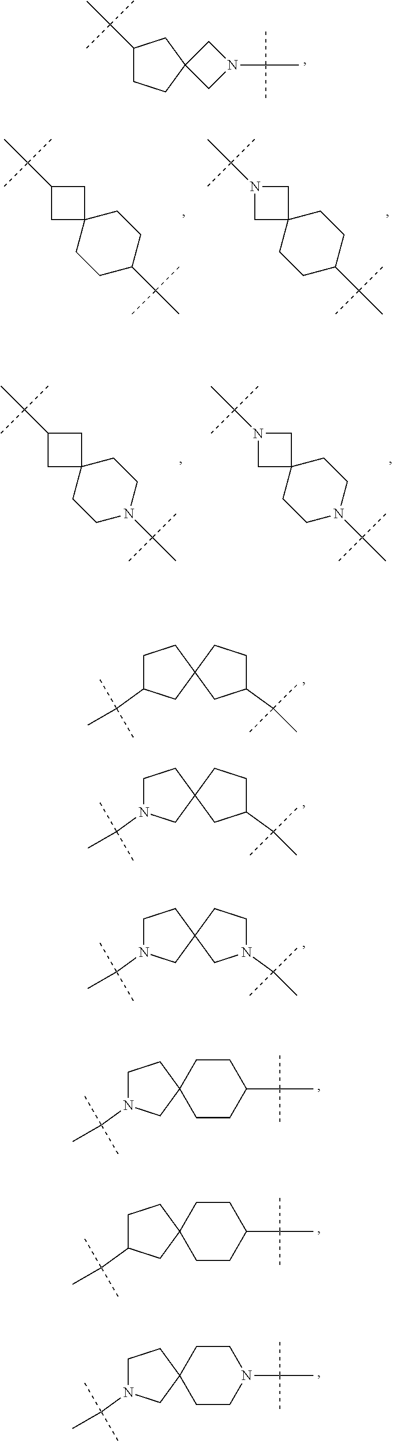

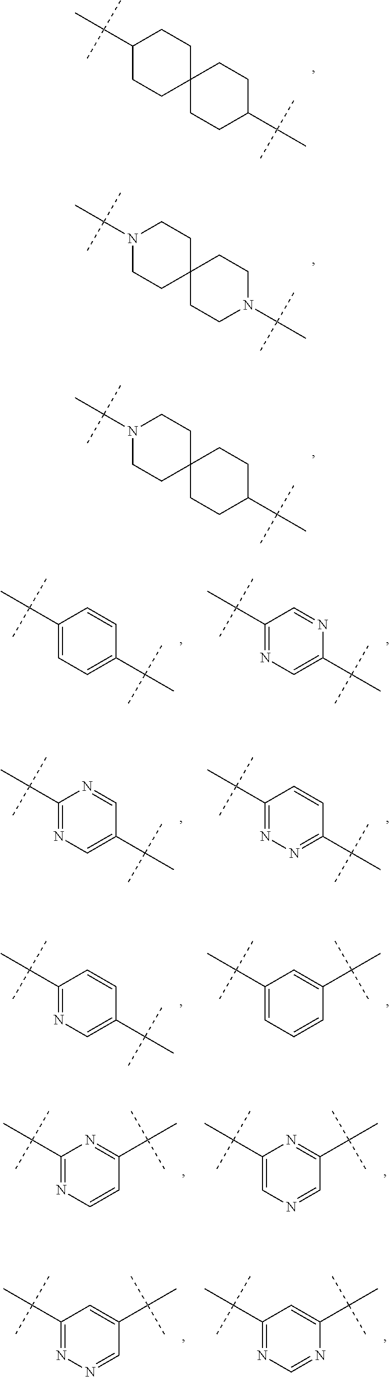

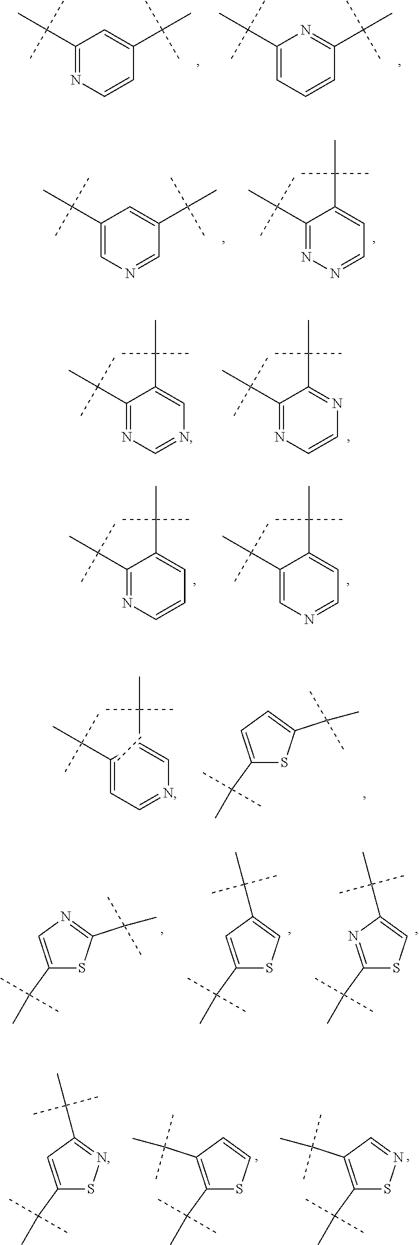

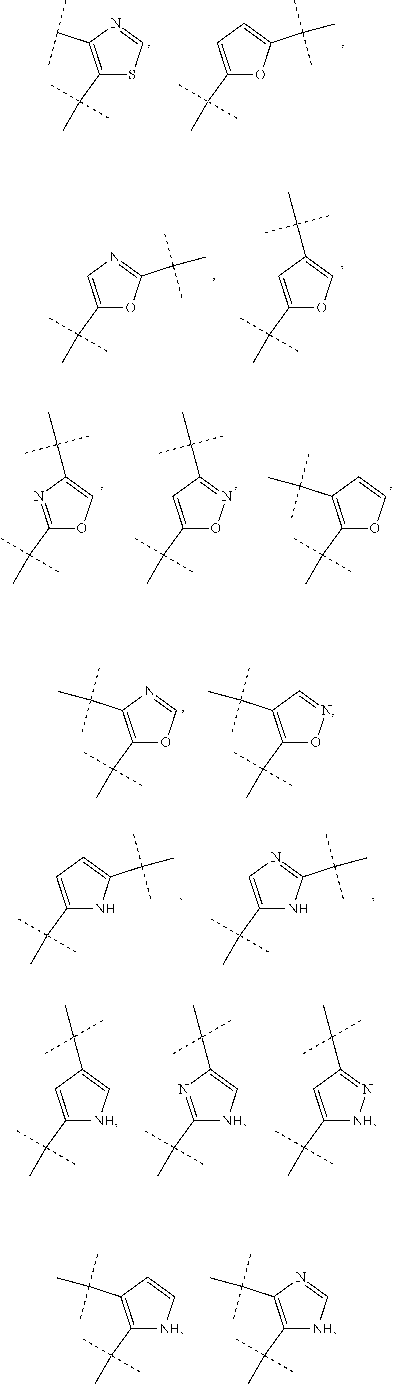

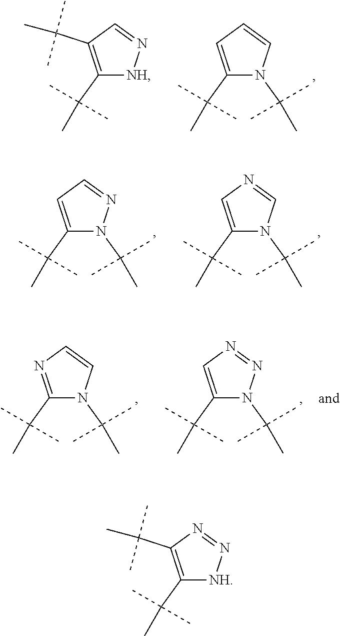

In another embodiment, the length of the linker is 0 to 40 atoms.

In another embodiment, the length of the linker is 0 to 20 atoms.

In another embodiment, the length of the linker is 0 to 10 atoms.

In another embodiment, the length of the linker is 0 to 40 atoms.

In another embodiment, the linker is selected from null, optionally substituted —CO—(CH₂)₀₋₁₀—, optionally substituted —(CH₂)₀₋₁₀—, optionally substituted —(CH₂)₁₋₂—(CO)NH—(CH₂)₀₋₁₀—, optionally substituted —(CH₂)₁₋₂—(CO)NH—(CH₂)₁₋₃—(OCH₂CH₂)₁₋₇—, optionally substituted —(CH₂)₀₋₁—CO—(CH₂)₁₋₃—(OCH₂CH₂)₁₋₇—, optionally substituted —CO—(CH₂)₀₋₃-(alkenylene)-(CH₂)₀₋₃—, optionally substituted —CO—(CH₂)₀₋₃-(alkynylene)-(CH₂)₀₋₃—, optionally substituted —CO—(CH₂)₀₋₃-(3-8 membered carbocyclic)-(CH₂)₀₋₃—, optionally substituted —CO—(CH₂)₀₋₃-(3-8 membered heterocyclic)-(CH₂)₀₋₃—, optionally substituted —(CH₂)₀₋₃-(alkenylene)-(CH₂)₀₋₃—, optionally substituted —(CH₂)₀₋₃-(alkynylene)-(CH₂)₀₋₃—, optionally substituted —(CH₂)₀₋₃-(3-8 membered carbocyclic)-(CH₂)₀₋₃—, optionally substituted —(CH₂)₀₋₃-(3-8 membered heterocyclic)-(CH₂)₀₋₃—, optionally substituted —(CH₂)₀₋₈—R^{r}—(CH₂)₀₋₈—, optionally substituted —(CH₂)₀₋₈—R—(CO)—(CH₂)₁₋₈—, optionally substituted —(CH₂)₀₋₈—R^{r}—(CH₂)₁₋₂—(CO)—NH—(CH₂)₂₋₉—, optionally substituted —(CH₂)₀₋₈—R^{r}—(CH₂)₁₋₂—(CO)—NH—(CH₂)₁₋₃—(OCH₂CH₂)₁₋₇—, optionally substituted —(CH₂)₀₋₈R^{r}—(CH₂)₀₋₁—(CO)—(CH₂)₁₋₃—(OCH₂CH₂)₁₋₇—, optionally substituted —(CH₂)₀₋₈—R—(CO)—(CH₂)₀₋₃-(alkenylene)-(CH₂)₀₋₃—, optionally substituted —(CH₂)₀₋₈—R—(CO)—(CH₂)₀₋₃-(alkynylene)-(CH₂)₀₋₃—, optionally substituted —(CH₂)₀₋₈—R^{r}—(CO)—(CH₂)₀₋₃-(3-8 membered carbocyclic)-(CH₂)₀₋₃—, optionally substituted —(CH₂)₀₋₈—R^{r}—(CO)—(CH₂)₀₋₃-(3-8 heterocyclic)-(CH₂)₀₋₃—, optionally substituted —(CH₂)₀₋₈—R^{r}—(CH₂)₀₋₃-(alkenylene)-(CH₂)₀₋₃—, optionally substituted —(CH₂)₀₋₈—R—(CH₂)₀₋₃-(alkynylene)-(CH₂)₀₋₃—, optionally substituted —(CH₂)₀₋₈—R^{r}—(CH₂)₀₋₃-(3-8 membered carbocyclcyl)-(CH₂)₀₋₃—, and optionally substituted —(CH₂)₀₋₈—R^{r}—(CH₂)₀₋₃-(heterocyclic)-(CH₂)₀₋₃—.

In some embodiments, the linker can also be a moiety of:

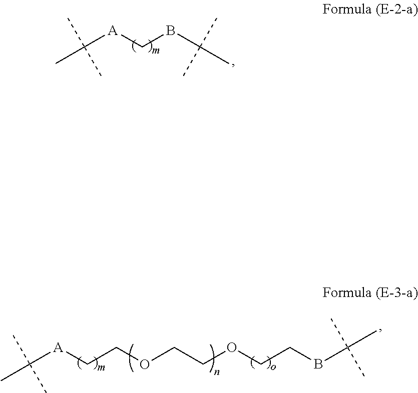

wherein
A is C═O or CH₂,
B is C═O or CH₂, and
m is 0-16,
n is 0-6,and
o is 0-6.

In some embodiments, the linker can also be a moiety of:

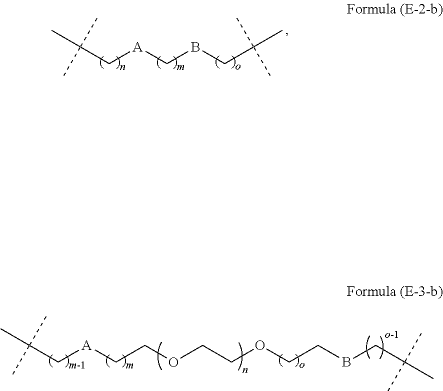

A is —C(O)N—,
B is —C(O)N—,
m is 0-16,
n is 0-6,
o is 0-6,
n-1 is 0-6, and
o-1 is 0-6.

### DUB Recruiter

In some aspects, the DUB recruiter comprises the structures of Formula (A-I):

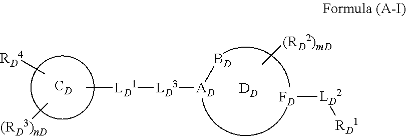

or a tautomer thereof, or a pharmaceutically acceptable salt of the compound or the tautomer,
wherein
R_{D}⁴ is R_{D}^{4b}.

In some embodiments, the DUB recruiters comprise the structures of Formulae (A-I-a), (A-I-a1), (A-I-a2), (A-I-a3), (A-I-a4), (A-I-a5), (A-I-a6), (A-I-a7), (A-I-a8), (A-I-a9), (A-I-a10), (A-I-a1 l), (A-I-b), (A-I-b1), (A-I-b2), (A-I-b3), (A-I-b4), (A-I-b5), (A-I-b6), (A-I-b7), (A-I-b8), (A-I-b9), (A-I-b10), (A-I-b11), (A-I-c), (A-I-c1), (A-I-c2), (A-I-c3), (A-I-c4), (A-I-c5), (A-I-c6), (A-I-c7), (A-I-c8), (A-I-c9), (A-I-c10), (A-I-c11), (A-I-c12), (A-I-c13), (A-I-c14), (A-I-c15), (A-I-c16), (A-I-d), (A-I-d1), (A-I-d2), (A-I-d3), (A-I-d4), (A-I-d5), (A-I-d6), (A-I-d7), (A-I-d8), (A-I-d9), (A-I-d10), and (A-I-b11), wherein R_{D}⁴ is R_{D}^{4b}.

In some embodiments, R_{D}^{4b} is a bond.

In some embodiments, the DUB recruiters comprise:

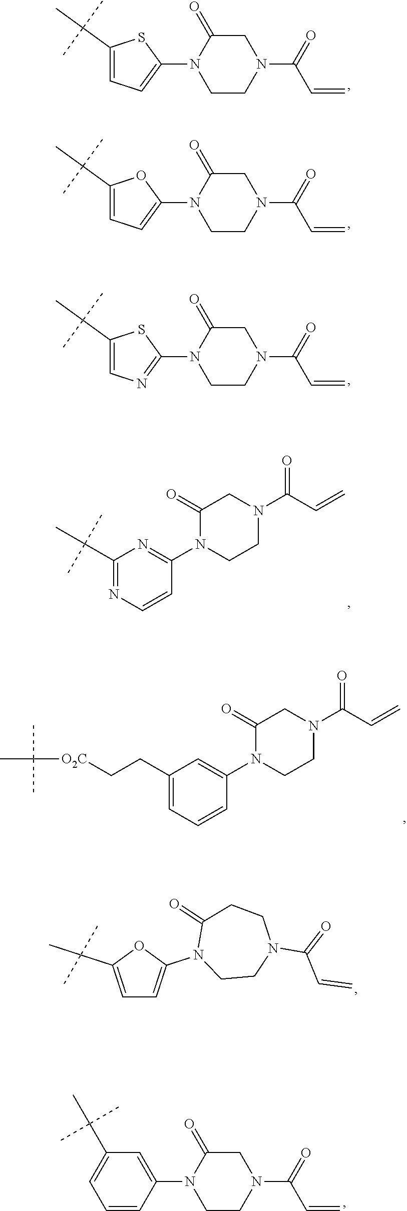

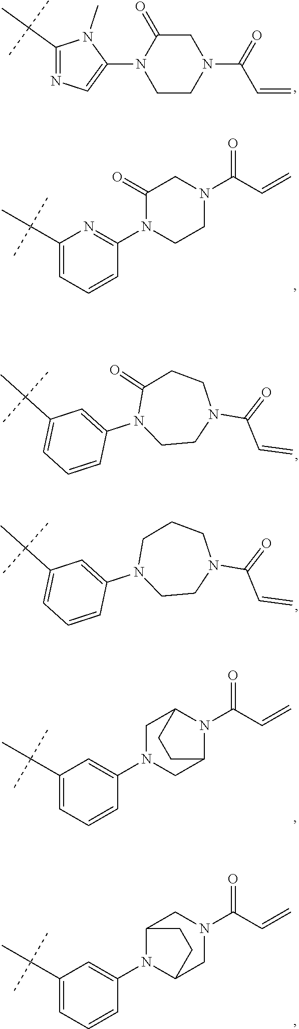

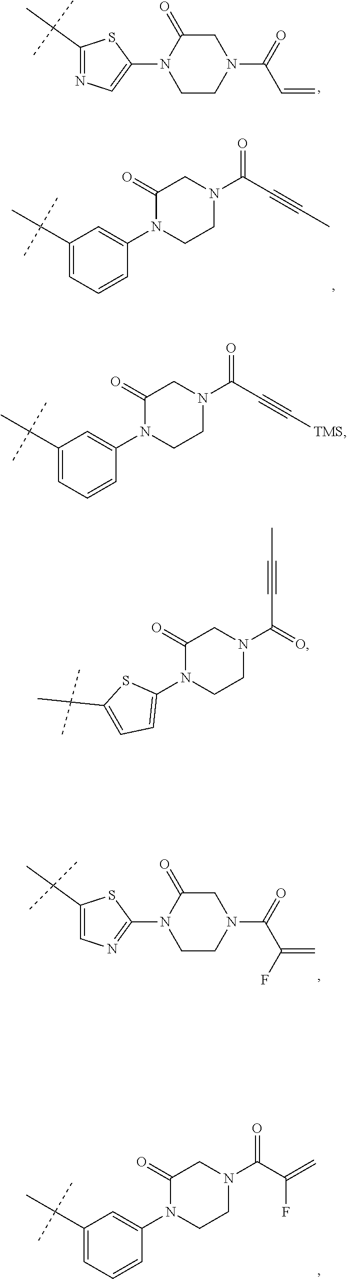

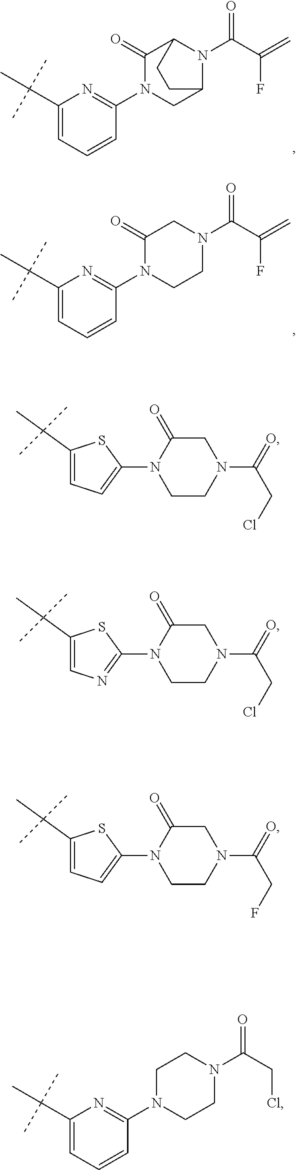

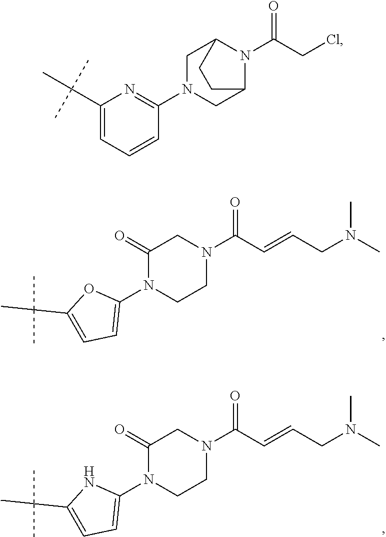

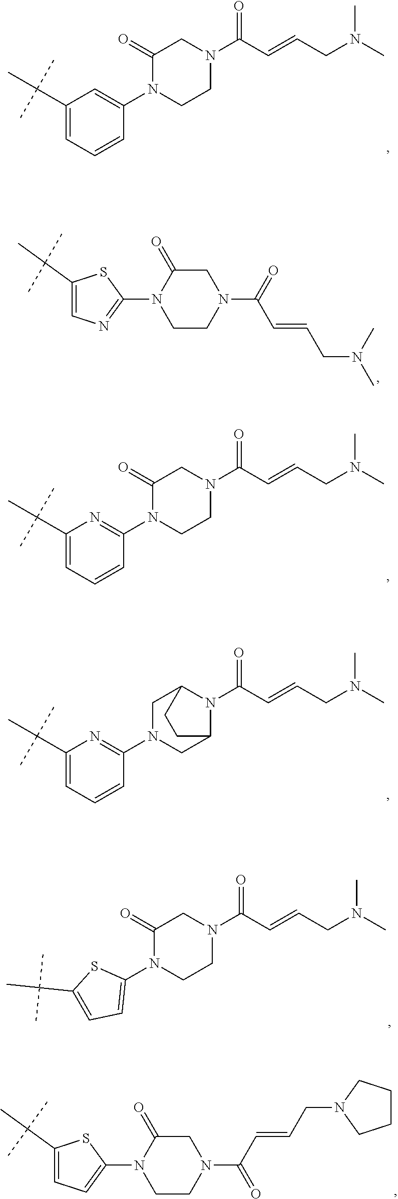

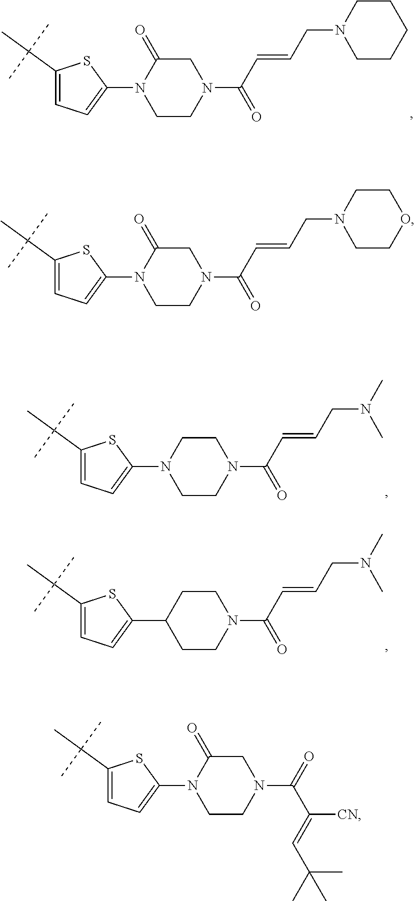

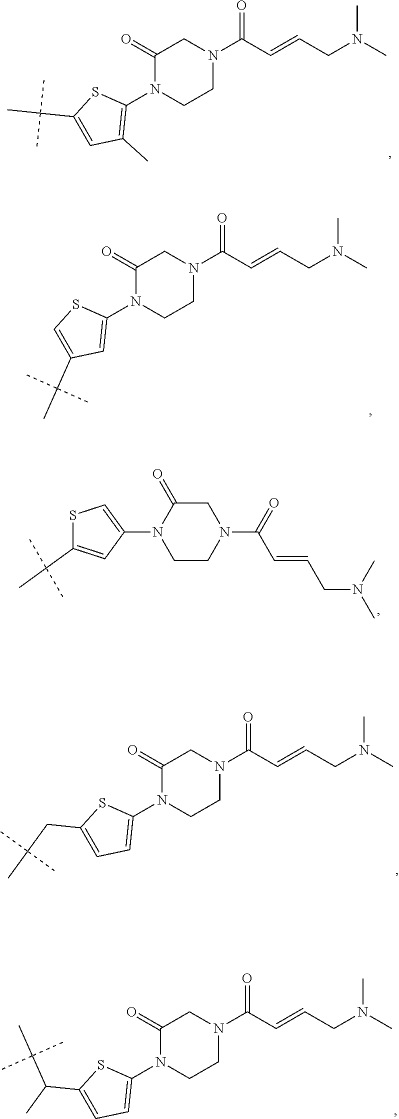

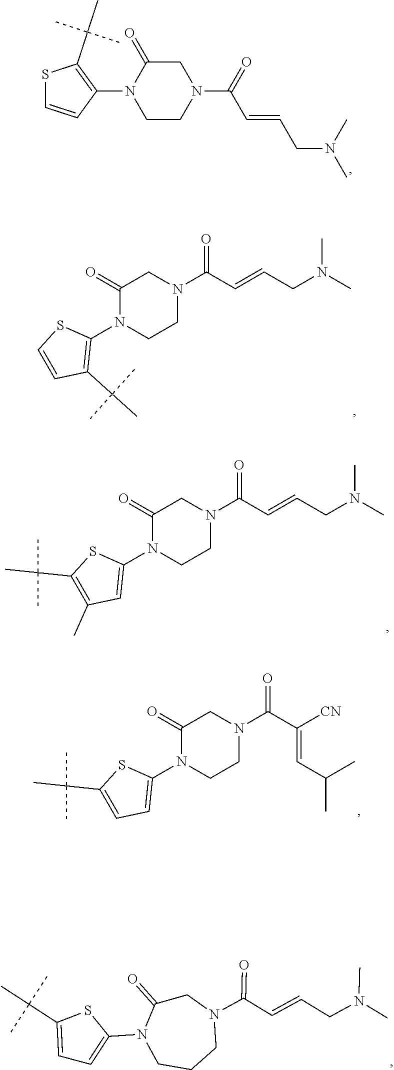

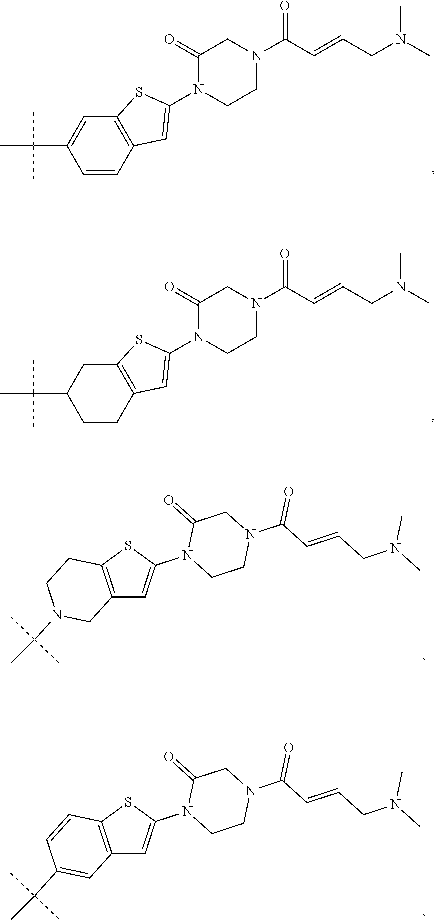

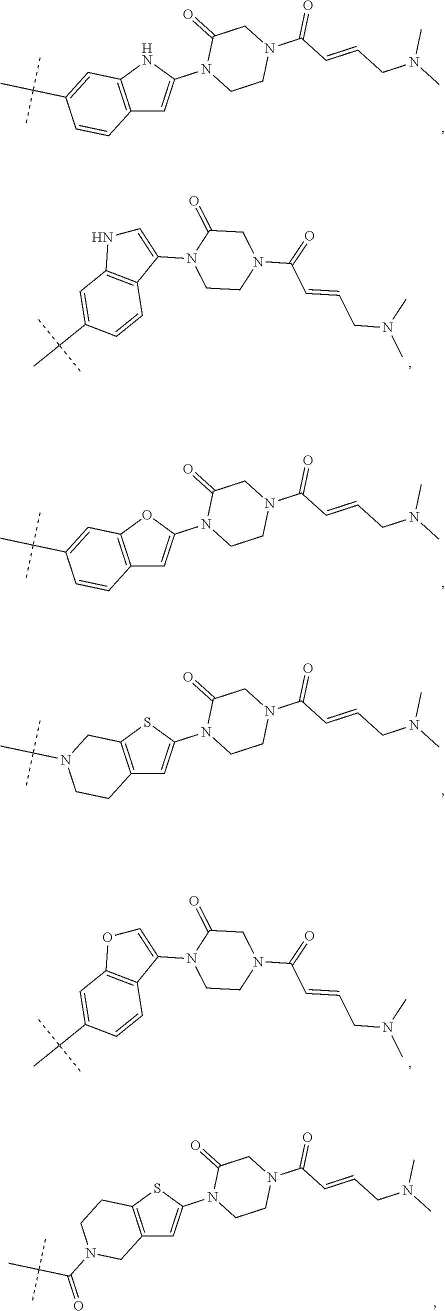

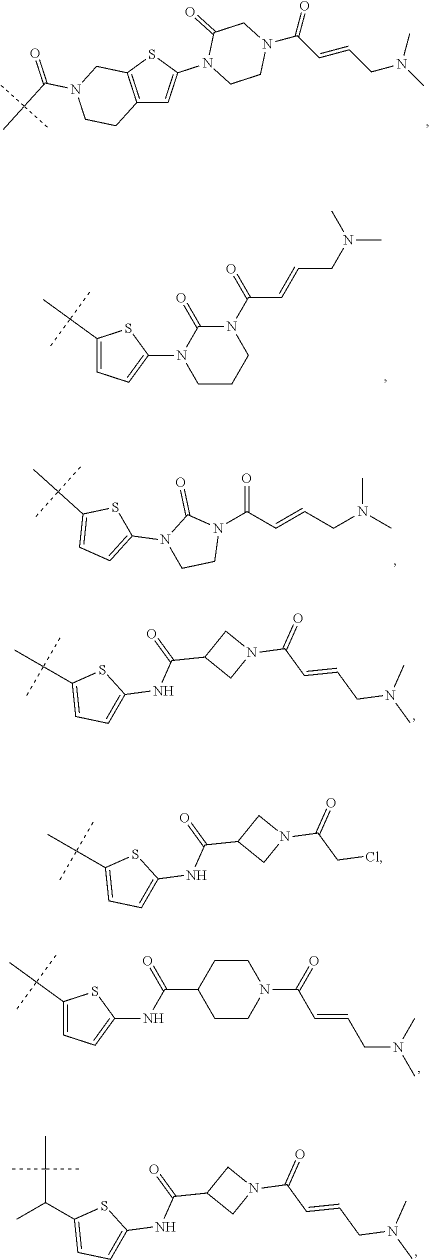

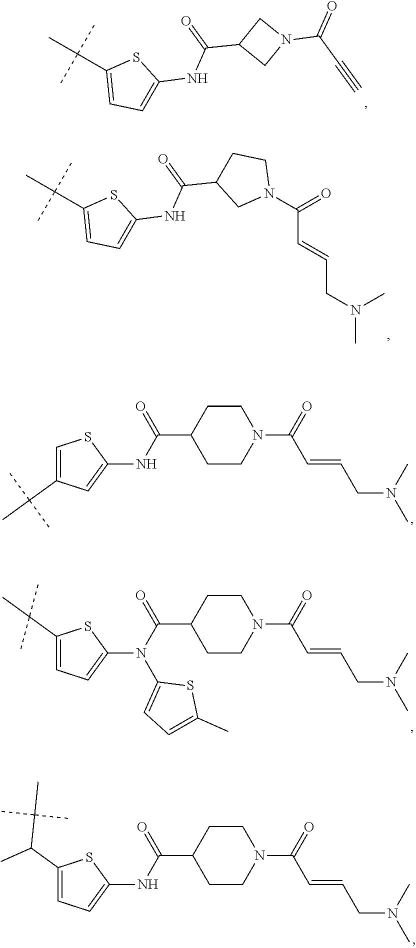

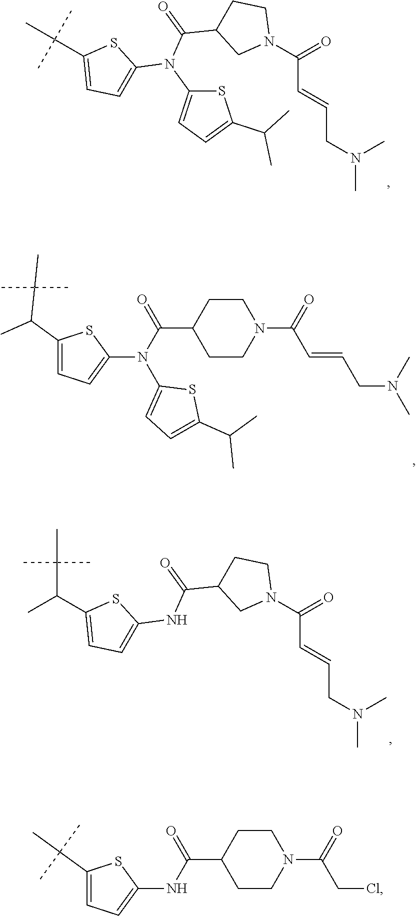

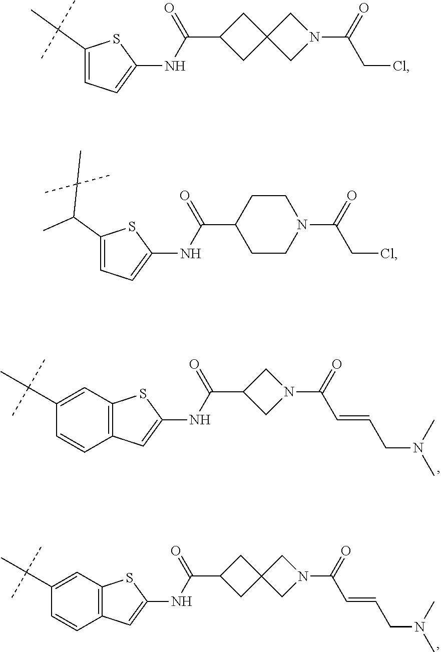

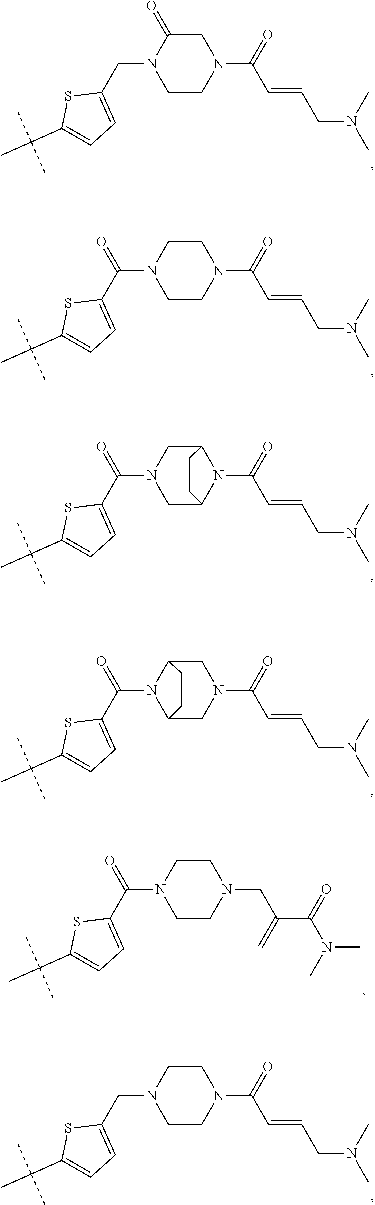

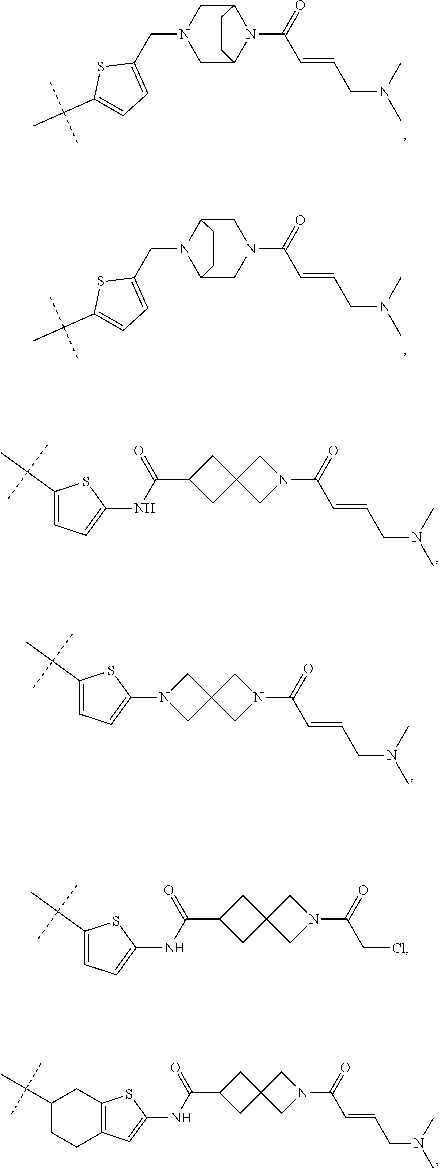

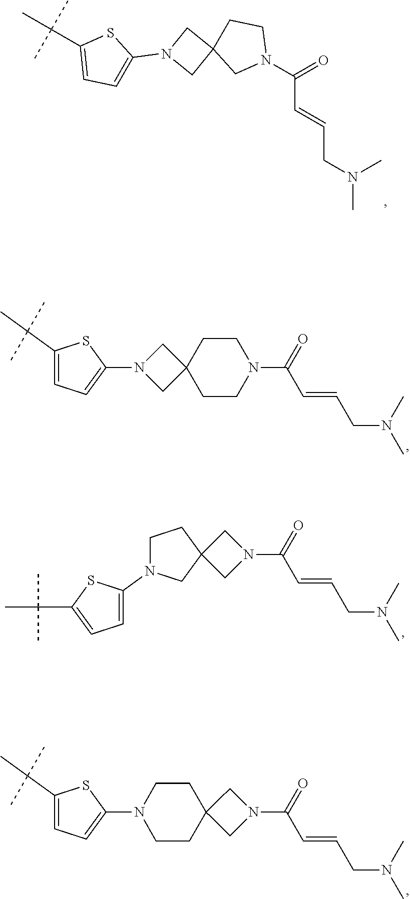

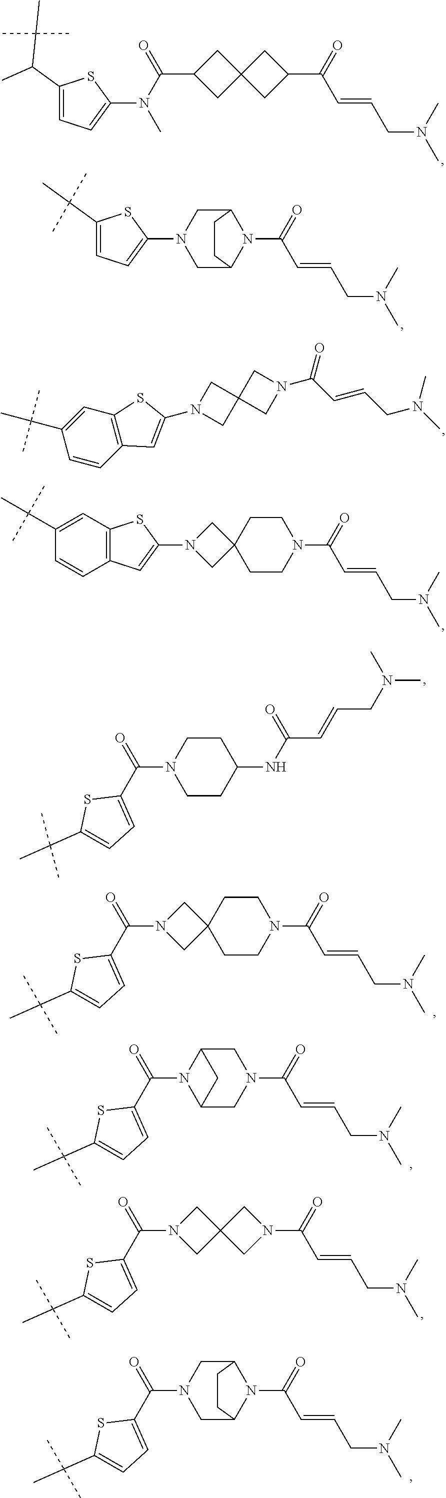

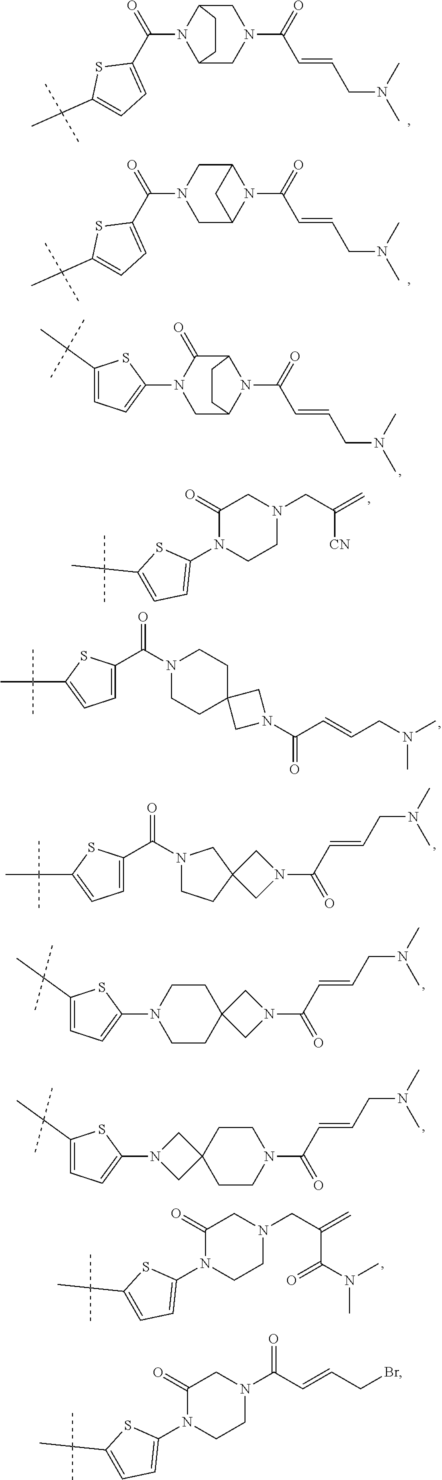

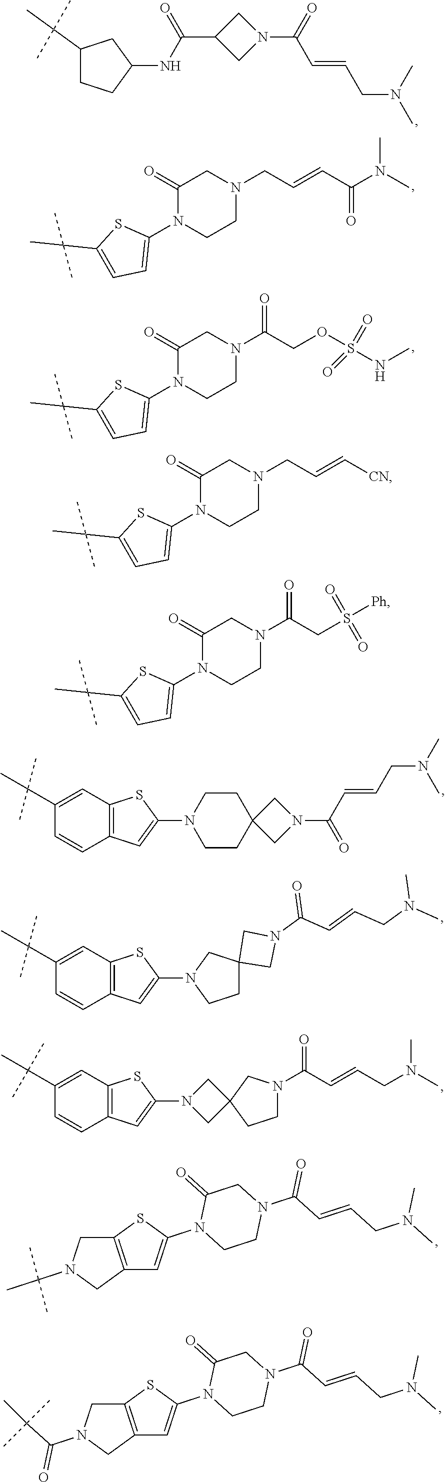

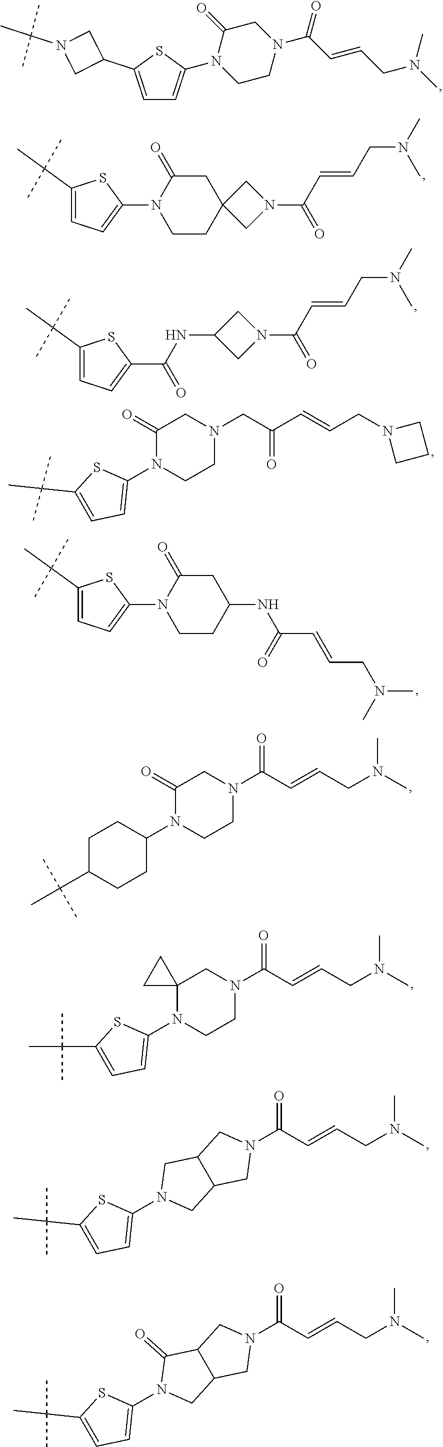

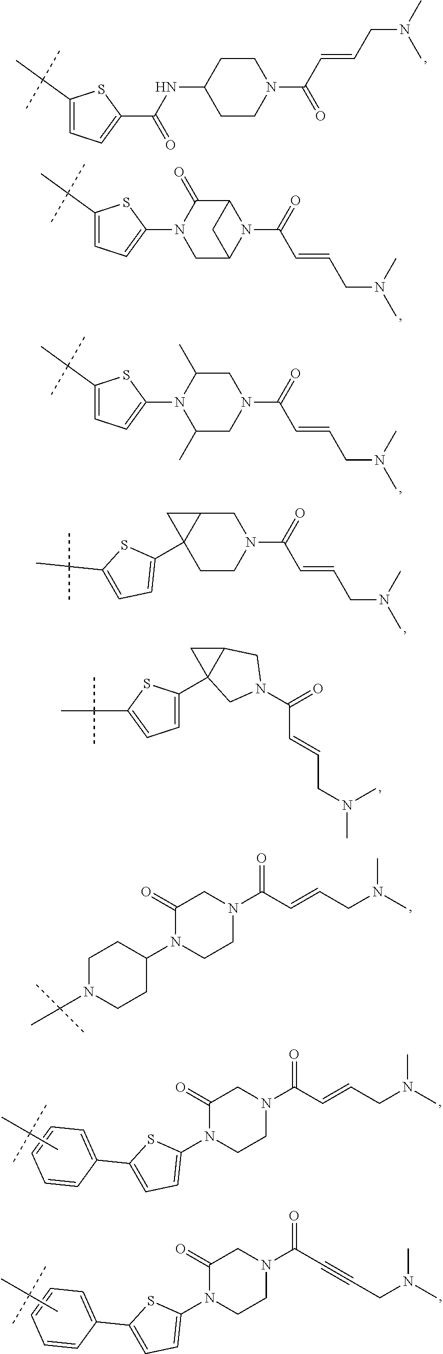

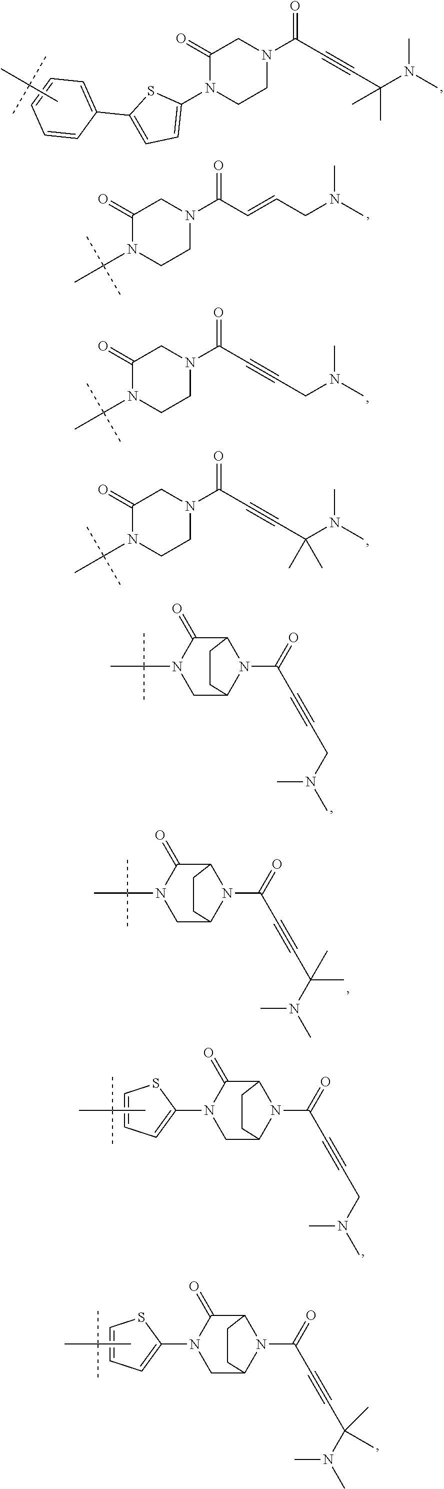

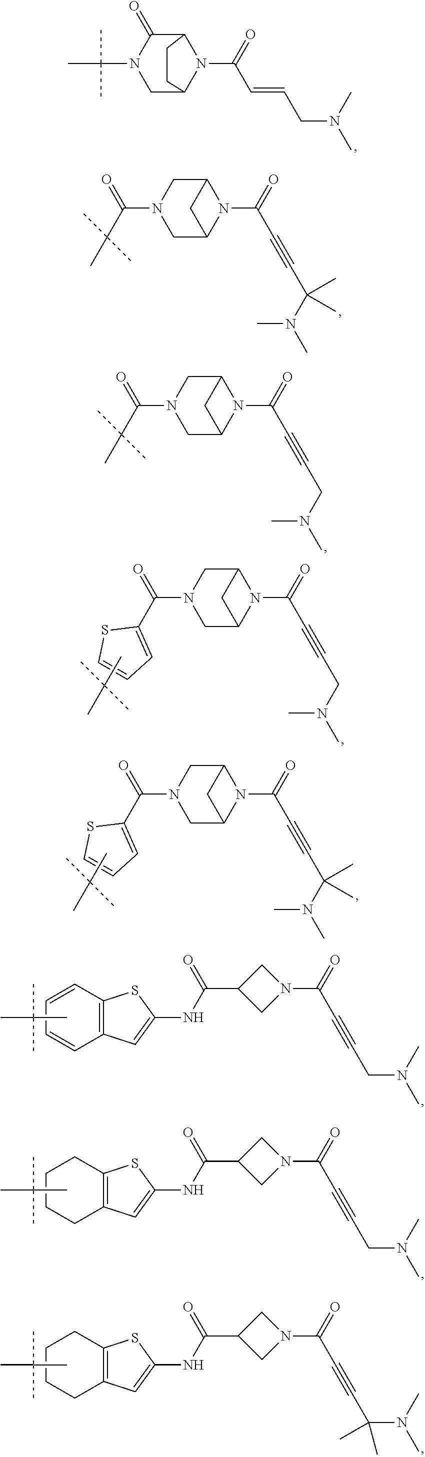

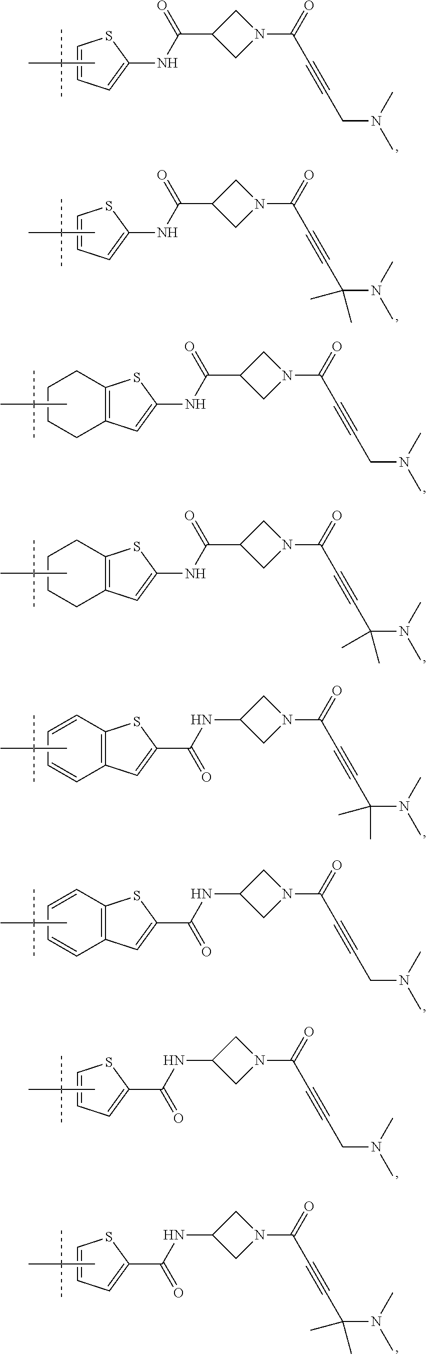

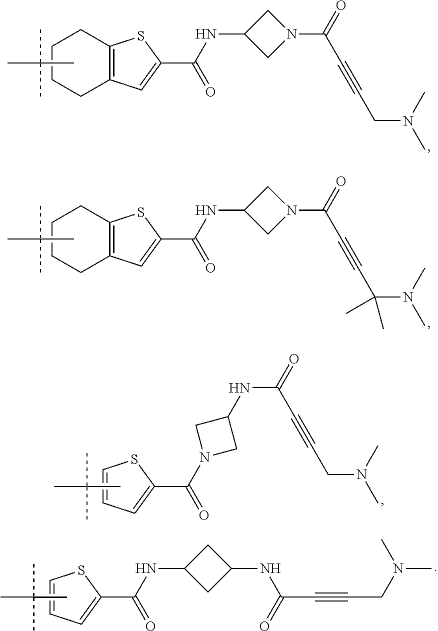

In some embodiments, the AMPK based bivalent compound is a compound selected from the following compounds, as identified in Table 3 below: XF137-81, XF137-82, XF137-83, XF137-84, XF137-85, XF137-86, XF137-87, XF137-88, XF137-89, XF137-90, XF137-91, XF137-92, XF137-93, XF137-94, QC179-047, QC179-048, QC137-049, QC179-050, QC179-051, QC179-052, QC179-053, QC179-054, QC137-055, and examples 367-380, or analogs thereof.

In some embodiments, the cGAS based bivalent compound is a compound selected from the following compounds, as identified in table 4 below: ZD178-22, ZD178-23, ZD178-24, ZD178-25, ZD178-26, ZD178-27, ZD178-28, ZD178-29, ZD178-30, ZD178-31, ZD178-32, ZD178-33, ZD178-34, ZD178-35, ZD178-58-1, ZD178-58-2, ZD178-58-3, ZD178-58-4, ZD178-58-5, ZD178-58-6, ZD178-58-7, ZD178-58-8, ZD178-58-9, ZD178-58-10, ZD178-58-11, ZD178-58-12, ZD178-58-13, ZD178-58-14, ZD178-63-1, ZD178-63-2, ZD178-63-3, ZD178-63-4, ZD178-63-5, ZD178-63-6, ZD178-63-78, ZD178-63-8, ZD178-63-9, ZD178-63-10, ZD178-63-11, ZD178-63-12, ZD178-63-13, ZD178-63-14, ZD178-63-15, ZD178-69-1, ZD178-69-2, ZD178-69-3, ZD178-69-4, ZD178-69-5, ZD178-69-6, ZD178-69-7, ZD178-69-8, ZD178-69-9, ZD178-69-10, ZD178-69-11, ZD178-69-12, ZD178-69-13, ZD178-69-14, ZD178-69-15, and examples 381-388, or analogs thereof.

In some embodiments, the CFTR based bivalent compound is a compound selected from the following compounds, as identified in Table 5 below: QC166-130, QC166-131, QC166-132, QC166-133, QC166-134, QC166-135, QC166-136, QC166-137, QC166-138, QC166-139, QC166-140, QC166-141, QC166-142, QC166-143, QC166-167, QC166-174, QC166-175, QC166-176, QC166-177, QC166-178, QC166-179, QC166-181, QC166-182, QC166-183, QC166-184, QC166-185, QC179-104, QC179-105, QC179-106, QC179-107, QC179-108, QC179-109, QC179-110, QC179-111, QC179-112, QC179-113, QC179-137, QC179-138, QC179-139, QC179-140, QC179-141, QC179-154, QC179-155, QC179-156, QC179-157, QC179-158, QC192-153, QC192-154, QC192-155, QC192-156, QC192-157, QC192-184, QC192-178, QC192-179, QC192-180, QC192-181, QC192-182, or analogs thereof.

In some embodiments, this disclosure provides a method of treating AMPK, cGAS or CFTR-mediated disease, the method including administering to a subject in need thereof one or more bivalent compounds including a AMPK, cGAS, or CFTR ligand conjugated to a OTUB1 binder via a linker. The AMPK, cGAS, CFTR-mediated disease can be a disease resulting from AMPK, cGAS or CFTR degradation. The AMPK, cGAS or CFTR-mediated disease can have decrease CFTR expression relative to a wild-type tissue of the same species and tissue type.

In some embodiments, the methods described herein, the bivalent compounds can be administered, e.g., orally, parenterally, intradermally, subcutaneously, topically, and/or rectally.

This disclosure additionally provides a method for identifying a bivalent compound which mediates de-ubiquitination/stabilization of AMPK, cGAS, or CFTR, the method including providing a heterobifunctional test compound including a AMPK, cGAS, or CFTR ligand conjugated to a de-ubiquitination tag via a linker, contacting the heterobifunctional test compound with a cell (e.g., a cell such as a AMPK, cGAS or CFTR-mediated disease cell) including a deubiquitinase (e.g., OTUB1) and AMPK, cGAS or CFTR protein.

As used herein, the terms “about” and “approximately” are defined as being within plus or minus 10% of a given value or state, preferably within plus or minus 5% of said value or state. The terms “bivalent” and “bi-functional” are used interchangeably herein. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1. Effect of exemplary compounds on modifying OTUB1 in mass spectrometry-based assays.

FIG. 2. Discovery of improved OTUB1 ligand MS5105.

FIG. 3. Effect of exemplary AMPK based bivalent compounds in stabilization AMPK protein levels in Hela and HEK293T cells

FIG. 4. Effect of exemplary AMPK based bivalent compounds in stabilization AMPK protein level in HEK293T cells in multiple concentrations.

FIG. 5. Effect of exemplary cGAS based bivalent compounds in stabilization cGAS protein level in Hela cells.

FIG. 6. Discovery of first-in-class cGAS DUBTACs.

FIG. 7. cGAS DUBTACs MS7829 and mS8588 activate the cGAS/STING/IRF3 signaling.

FIG. 8. The MS5105-based DUBTAC MS6178 increases CFTR-ΔF508 protein level much more effectively than the EN523-based CFTR DUBTAC NJH-2-057.

FIG. 9. Effect of exemplary CFTR based bivalent compounds on stabilizing CFTR protein levels in human cystic fibrosis bronchial epithelial cells.

FIG. 10. Effect of exemplary CFTR or cGAS based bivalent compounds on modifying OTUB1 mass spectrometry-based assays.

### Testing of Exemplary Compounds

The activity of novel synthesized covalent OTUB1 binder and bivalent AMPK-OTUB1, cGAS-OTUB1, CFTR-OTUB1 compounds can be assessed using standard biophysical assays (e.g., isothermal titration calorimetry (ITC), surface plasmon resonance (SPR)) and mass spectrometry-based assays. Cellular assays can then be used to assess the bivalent CFTR-OTUB1 compound's ability to stabilize AMPK, cGAS or CFTR proteins level. Assays suitable for use in any or all of these steps are known in the art, and include, e.g., Western blotting, quantitative mass spectrometry (MS) analysis, flow cytometry, enzymatic inhibition, ITC, SPR, cell growth inhibition and xenograft and PDX models.

By way of non-limiting example, detailed synthesis protocols are described in the Examples for specific exemplary covalent OTUB1 binders, bivalent AMPK-OTUB1, cGAS-OTUB1 and CFTR-OTUB1 compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²p ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

### Definition of Terms

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkylene” refers to a bivalent saturated aliphatic radical (such as ethylene) regarded as derived from an alkene by opening of the double bond or from an alkane by removal of two hydrogen atoms from different carbon atoms.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “alkenylene” refers to linear or branched-chain divalent hydrocarbon radical of two to eight carbon atoms (C₂-C₈) with at least one site of unsaturation, i.e., a carbon-carbon, SP2 double bond, wherein the alkenylene radical may be optionally substituted independently with one or more substituents described herein, and includes radicals having “cis” and “trans” orientations, or alternatively, “E” and “Z” orientations. Examples include, but are not limited to, ethylenylene or vinylene (—CH═CH—), allyl (—CH2CH═CH—), and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkynylene” refers to a linear or branched divalent hydrocarbon radical of two to eight carbon atoms (C₂-C₈) with at least one site of unsaturation, i.e., a carbon-carbon, sp triple bond, wherein the alkynylene radical may be optionally substituted independently with one or more substituents described herein. Examples include, but are not limited to,ethynylene (—C≡C—), propynylene (propargylene, —CH2C≡C—), and the like.

The term “alkoxy”, as used herein, means an alkyl group as defined herein witch is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

“Heteroalkyl” refers to a substituted or unsubstituted alkyl group which has one or more skeletal chain atoms selected from an atom other than carbon, Exemplary skeletal chain atoms selected from an atom other tha n carbon include, e.g., O, N, P, Si, S, or combinations thereof, wherein the nitrogen, phosphorus, and sulfur atoms may optionally be oxidized and the nitrogen heteroatom may optionally be quaternized. If given, a numberical range refers to the chain length in total. For example, a 3- to 8-membered heteroalkyl has a chain length of 3 to 8 atoms. Connection to the rest of the molecule may be through either a heteroatom or a carbon in the heteroalkyl chain. Unless stated otherwise specifically in the specification, a heteroalkyl group is optionally substituted by one or more substituents such as those substituents described herein.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hickel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “arylene” means a divalent aromatic hydrocarbon radical of 6-20 carbon atoms (C6-C20) derived by the removal of two hydrogen atom from two carbon atoms of a parent aromatic ring system. Some arylene groups are represented in the exemplary structures as “Ar”. Arylene includes bicyclic radicals comprising an aromatic ring fused to a saturated, partially unsaturated ring, or aromatic carbocyclic ring. Typical arylene groups include, but not limited to, radicals derived from benzene (phenylene), substituted benzenes, naphthalene, anthracene, biphenylene indenylene, indaylene, 1,2-dihydronaphthalene, 1,2,3,4,-tetrahydronaphthyl, and the like. Arylene groups are optionally substituted with one or more substituents described herein.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Huckel theory. Heteroaryl includes fused or bridged ring systems. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl,pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclic”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclic group may include fused, bridged or spirocyclic ring systems. In certain embodiments, a heterocyclic group comprises 3 to 10 ring atoms (3-10 membered heterocyclic). In certain embodiments, a heterocyclic group comprises 3 to 8 ring atoms (3-8 membered heterocyclic). In certain embodiments, a heterocyclic group comprises 4 to 8 ring atoms (4-8 membered heterocyclic). In certain embodiments, a heterocyclic group comprises 3 to 6 ring atoms (3-6 membered heterocyclic). A heterocyclic group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclic group contains a sulfur atom, said sulfur atom may be oxidized with one or two oxygen atoms to afford either a sulfoxide or sulfone. An example of a 4 membered heterocyclic group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclic groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclic” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” or “carbocyclicene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂ alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

As used herein, the same symbol in a different FORMULA may have a different definition, for example, the definition of R¹ in FORMULA 1 is as defined with respect to FORMULA 1 and the definition of R¹ in FORMULA 6 is as defined with respect to FORMULA 6.

As used herein, when m (or n or u or v or w) is defined by a range, for example, “m is 0 to 15” or “m=0-3” means that m is an integer from 0 to 15 (i.e. m is 0, 1, 2, 3, 4, 5, 6, 7, 8, 9, 10, 11, 12, 13, 14, or 15) or m is an integer from 0 to 3(i.e. m is 0, 1,2, or 3) or is any integer in the defined range.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art. When co-administered, OTUB1 covalent binders or AMPK, cGAS or CFTR based bivalent compounds disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the OTUB1 covalent binders or AMPK, cGAS or CFTR based bivalent compounds or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as a-, p-, and y-cyclodextrin, may also be advantageously used to enhance delivery of compounds of the formulae described herein.

As used herein, the OTUB1 covalent binders or AMPK, cGAS or CFTR based bivalent compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The OTUB1 covalent binders or AMPK, cGAS or CFTR based bivalent compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In particular, pharmaceutically acceptable salts of the OTUB1 covalent binders or AMPK, cGAS or CFTR based bivalent compounds disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium and N-(alkyl)4+ salts. The invention also envisions the quaternization of any basic nitrogen-containing groups of the OTUB1 covalent binders or AMPK, cGAS and CFTR based bivalent compounds disclosed herein. Water or oil-soluble or dispersible products can be obtained by such quaternization.

In some aspects, the pharmaceutical compositions disclosed herein can include an effective amount of one or more OTUB1 covalent binders or AMPK, cGAS and CFTR based bivalent compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., an OTUB1 covalent binder or an AMPK, cGAS or CFTR based bivalent compound) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising an OTUB1 covalent binder or AMPK, cGAS or CFTR based bivalent compound, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., AMPK, cGAS or CFTR-related disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. Exemplary AMPK, cGAS or CFTR-mediated diseases that can be treated with AMPK, cGAS or CFTR based DUBTACs include, for example, cystic fibrosis, breast cancer, ovarian cancer, prostate cancer, colon cancer, pancreatic cancer, bladder cancer, liver cancer and cervical cancer.

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Procedures for the Synthesis of OTUB1 Covalent Binders

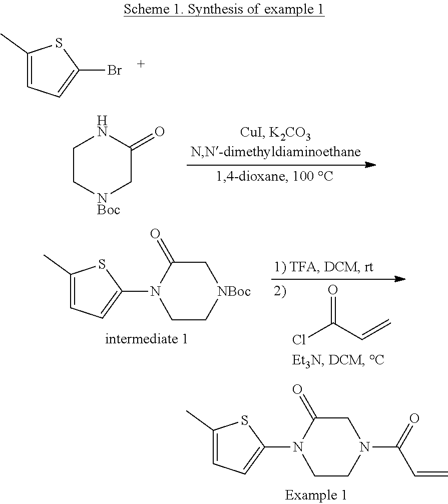

Example 1: 4-acryloyl-1-(5-methylthiophen-2-yl)piperazin-2-one (XS154-91) To a solution of 2-bromo-5-methylthiophene (50.0 mg, 0.28 mmol) dissolved in dioxane (1 mL), N,N′-dimethylethylenediamine (9 μL, 0.0846 mmol, 0.3 eq), K₂CO₃ (116.9 mg, 0.846 mmol, 3.0 eq), CuI (5.4 mg, 0.028 mmol, 0.1 eq) followed by tert-butyl 3-oxopiperazine-1-carboxylate (84.7 mg, 0.42 mmol, 1.5 eq). The reaction mixture was stirred at 100° C. under nitrogen atmosphere overnight. After cooling down to rt, resulting crude mixtures were purified via silica gel column chromatography to yield intermediate 1.

To a solution of intermediate 1 (20.4 mg, 0.069 mmol) dissolved in DCM (0.5 mL), and the TFA(0.5 mL) were added. The reaction mixture stirred at rt 1 h. Excess TFA was removed, resulting crude product was re-dissovled in DCM (1 mL), Et₃N (13 μL, 0.089 mmol, 2.0 eq) was added. At 0° C. the acryloyl chloride (4.8 μL, 0.053 mmol, 1.2 eq.) was added, the reaction mixture stirred at 0° C. for 30 mins. Resulting crude mixtures were purified via prep-HPLC to yield title compound (White solid, 55% yield) ¹H NMR (400 MHz, Methanol-d₄) δ 6.92-6.68 (m, 1H), 6.68-6.54 (m, 2H), 6.31 (d, J=16.7 Hz, 1H), 5.90-5.76 (m, 1H), 4.48 (d, J=28.0 Hz, 2H), 4.15-3.81 (m, 4H), 2.42 (d, J=2.2 Hz, 3H). MS (ESI) [M+H]⁺=251.0.

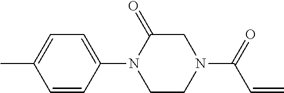 [SMILES: C=CC(=O)N1CCN(c2ccc(C)cc2)C(=O)C1]

Example 2: 4-acryloyl-1-(p-tolyl)piperazin-2-one (XS154-130). Example 2 was synthesized following similar procedure for preparing example 1. White solid, 32% yield. ¹H NMR (400 MHz, Methanol-d4) δ 7.31-7.20 (m, 4H), 6.92-6.69 (m, 1H), 6.33 (dt, J=16.7, 2.4 Hz, 1H), 5.85 (d, J=10.6 Hz, 1H), 4.54-4.30 (m, 2H), 4.12-3.97 (m, 2H), 3.88-3.74 (m, 2H), 2.37 (d, J=2.2 Hz, 3H). MS (ESI) [M+H]⁺=245.4.

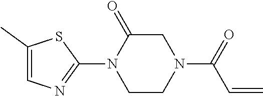 [SMILES: C=CC(=O)N1CCN(c2ncc(C)s2)C(=O)C1]

Example 3: 4-acryloyl-1-(5-methylthiazol-2-yl)piperazin-2-one (XS154-148). Example 3 was synthesized following similar procedure for preparing example 1. White solid, 26% yield. ¹H NMR (400 MHz, Chloroform-d) δ 8.19 (s, 1H), 6.70-6.49 (m, 1H), 6.48-6.33 (m, 1H), 5.98-5.73 (m, 1H), 4.73-4.45 (m, 2H), 4.30 (s, 2H), 4.14-3.91 (m, 2H), 2.44 (d, J=2.3 Hz, 3H). MS (ESI) [M+H]⁺=252.1.

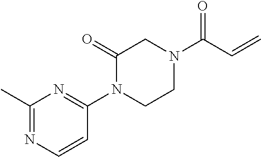 [SMILES: C=CC(=O)N1CCN(c2ccnc(C)n2)C(=O)C1]

Example 4: 4-acryloyl-1-(2-methylpyrimidin-4-yl)piperazin-2-one (XS154-149). Example 4 was synthesized following similar procedure for preparing example 1. White solid, 46% yield. ¹H NMR (400 MHz, Chloroform-d) δ 8.68 (d, J=6.3 Hz, 1H), 8.35 (s, 1H), 6.54 (t, J=13.2 Hz, 1H), 6.44 (d, J=16.4 Hz, 1H), 5.85 (dd, J=10.0, 2.6 Hz, 1H), 4.64-4.45 (m, 2H), 4.44-4.26 (m, 2H), 4.05-3.87 (m, 2H), 2.77 (d, J=2.2 Hz, 3H). MS (ESI) [M+H]=247.3.

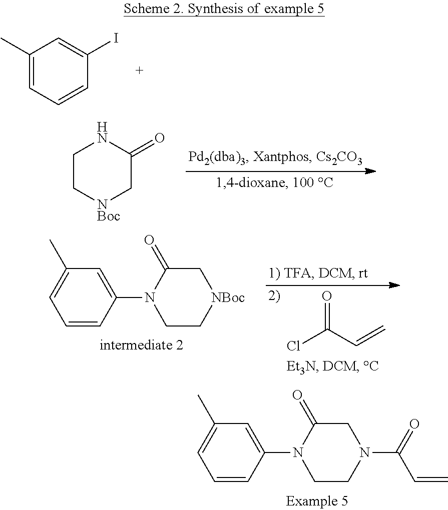

Example 5: 1-(4-(m-tolyl)piperazin-1-yl)prop-2-en-1-one (XS159-19). To a solution of the 1-iodo-3-methylbenzene (218.0 mg, 1 mmol) dissolved in dioxane (3 mL), Xantphos (173 mg, 0.3 mmol, 0.3 eq), Cs₂CO₃ (655.6 mg, 2.0 eq), Pd₂(dba)₃ (91.6 mg, 0.1 mmol, 0.1 eq), followed by tert-butyl 3-oxopiperazine-1-carboxylate (279.6 mg, 1.5 mmol, 1.5 eq). The reaction mixture was stirred at 100° C. under nitrogen atmosphere overnight. After cooling down to rt, resulting crude mixtures were purified via silica gel column chromatography to yield intermediate 2.

To a solution of intermediate 2 (260 mg, 0.94 mmol) dissolved in DCM (1 mL), and the TFA(1 mL) were added. The reaction mixture stirred at rt 1 h. Excess TFA was removed, resulting crude product was re-dissovled in DCM (1 mL), Et₃N (380 μL, 2.68 mmol, 3.0 eq) was added. At 0° C. the acryloyl chloride (87 μL, 1.07 mmol, 1.2 eq.) was added, the reaction mixture stirred at 0° C. for 30 mins. Resulting crude mixtures were purified via prep-HPLC to yield title compound (White solid, 17% yield) ¹H NMR (400 MHz, DMSO-d₆) δ 7.21-7.07 (m, 1H), 6.91-6.76 (m, 3H), 6.71 (d, J=7.6 Hz, 1H), 6.14 (dd, J=16.6, 3.1 Hz, 1H), 5.71 (dd, J=10.4, 3.0 Hz, 1H), 3.78-3.65 (m, 4H), 3.17 (s, 4H), 2.26 (d, J=2.3 Hz, 3H). MS (ESI) [M+H]⁺=231.1

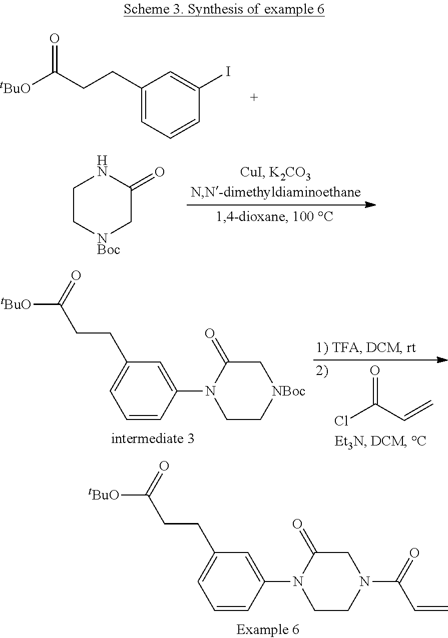

Example 6: 3-(3-(4-acryloyl-2-oxopiperazin-1-yl)phenyl)propanoic acid (XS159-13). To a solution of tert-butyl 3-(3-iodophenyl)propanoate (290.1 mg, 1 mmol) dissolved in dioxane (3 mL), N,N′-dimethylethylenediamine (32 μL, 0.3 mmol, 0.3 eq), K₂CO₃(414.6 mg, 3.0 mmol, 3.0 eq), CuI (19.1 mg, 0.1 mmol, 0.1 eq) followed by tert-butyl 3-oxopiperazine-1-carboxylate (300.3 mg, 1.5 mmol, 1.5 eq). The reaction mixture was stirred at 100° C. under nitrogen atmosphere overnight. After cooling down to rt, resulting crude mixtures were purified via silica gel column chromatography to yield intermediate 3.

To a solution of intermediate 3 (130 mg, 0.36 mmol) dissolved in DCM (0.5 mL), and the TFA(0.5 mL) were added. The reaction mixture stirred at rt 1 h. Excess TFA was removed, resulting crude product was re-dissovled in DCM (1 mL), Et₃N (68 μL, 0.483 mmol, 3.0 eq) was added. At 0° C. the acryloyl chloride (16 μL, 0.194 mmol, 1.2 eq.) was added, the reaction mixture stirred at 0° C. for 30 mins. Resulting crude mixtures were purified via prep-HPLC to yield title compound (White solid, 37% yield) ¹H NMR (400 MHz, Methanol-d₄) δ 7.35 (t, J=6.3 Hz, 1H), 7.24-7.16 (m, 3H), 6.90-6.66 (m, 1H), 6.32 (dd, J=16.9, 3.7 Hz, 1H), 5.84 (d, J=10.5 Hz, 1H), 4.43 (d, J=25.3 Hz, 2H), 4.04 (t, J=5.9 Hz, 2H), 3.90-3.77 (m, 2H), 2.95 (q, J=7.1, 6.5 Hz, 2H), 2.63 (q, J=7.5, 5.6 Hz, 2H). MS (ESI) [M+H]⁺=303.0.

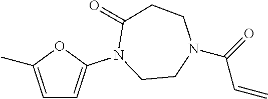 [SMILES: C=CC(=O)N1CCC(=O)N(c2ccc(C)o2)CC1]

Example 7: 1-acryloyl-4-(5-methylfuran-2-yl)-1,4-diazepan-5-one (XS159-107). Example 7 was synthesized following similar procedure for preparing example 1. White solid, 27% yield. ¹H NMR (400 MHz, Methanol-d₄) δ 7.43-6.67 (m, 2H), 6.23 (d, J=17.0 Hz, 1H), 6.03 (d, J=13.7 Hz, 1H), 5.79 (s, 1H), 3.92-3.76 (m, 6H), 2.73-2.63 (m, 2H), 2.25 (s, 3H). MS (ESI) [M+H]⁺=249.2

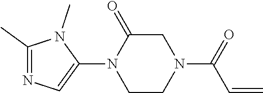 [SMILES: C=CC(=O)N1CCN(c2cnc(C)n2C)C(=O)C1]

Example 8: 4-acryloyl-1-(1,2-dimethyl-1H-imidazol-5-yl)piperazin-2-one (XS165-30). Example 8 was synthesized following similar procedure for preparing example 1. White solid, 19% yield. ¹H NMR (400 MHz, Chloroform-d) δ 7.38 (d, J=20.7 Hz, 1H), 6.65-6.41 (m, 1H), 6.34 (d, J=16.6 Hz, 1H), 5.79 (d, J=10.4 Hz, 1H), 4.41 (s, 2H), 3.98 (d, J=19.4 Hz, 4H), 3.69 (s, 3H), 2.58 (s, 3H). MS (ESI) [M+H]⁺=235.3.

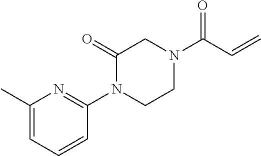 [SMILES: C=CC(=O)N1CCN(c2cccc(C)n2)C(=O)C1]

Example 9: 4-acryloyl-1-(6-methylpyridin-2-yl)piperazin-2-one (XS159-90). Example 9 was synthesized following similar procedure for preparing example 1. White solid, 53% yield. ¹H NMR (400 MHz, Chloroform-d) δ 7.82-7.65 (m, 1H), 7.60 (t, J=7.8 Hz, 1H), 6.98 (d, J=7.5 Hz, 1H), 6.63-6.46 (m, 1H), 6.40 (dd, J=16.7, 1.9 Hz, 1H), 5.89-5.69 (m, 1H), 4.44 (d, J=27.3 Hz, 2H), 4.20 (d, J=29.6 Hz, 2H), 3.94 (d, J=25.4 Hz, 2H), 2.49 (s, 3H). MS (ESI) [M+H]⁺=246.4.

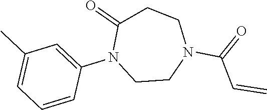 [SMILES: C=CC(=O)N1CCC(=O)N(c2cccc(C)c2)CC1]

Example 10: 1-acryloyl-4-(m-tolyl)-1,4-diazepan-5-one(XS165-53). Example 10 was synthesized following similar procedure for preparing example 1. White solid, 58% yield. ¹H NMR (400 MHz, Methanol-d4) δ 7.25 (t, J=7.4 Hz, 1H), 7.09 (d, J=7.7 Hz, 1H), 7.02 (s, 1H), 6.98 (d, J=8.0 Hz, 1H), 6.88-6.66 (m, 1H), 6.22 (dt, J=16.8, 1.9 Hz, 1H), 5.76 (t, J=8.5 Hz, 1H), 3.95-3.79 (m, 6H), 2.95-2.78 (m, 2H), 2.32 (s, 3H). MS (ESI) [M+H]⁺=259.2.

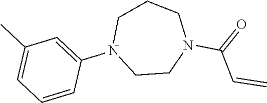 [SMILES: C=CC(=O)N1CCCN(c2cccc(C)c2)CC1]

Example 11: 1-(4-(m-tolyl)-1,4-diazepan-1-yl)prop-2-en-1-one(XS165-38). Example 11 was synthesized following similar procedure for preparing example 5. White solid, 15% yield. ¹H NMR (400 MHz, Chloroform-d) δ 7.21 (t, J=8.7 Hz, 1H), 6.89-6.47 (m, 4H), 6.35 (dd, J=59.9, 16.7 Hz, 1H), 5.76 (dd, J=29.7, 10.5 Hz, 1H), 3.87 (d, J=50.3 Hz, 2H), 3.71-3.40 (m, 6H), 2.35 (d, J=6.0 Hz, 3H), 2.17 (q, J=6.7 Hz, 2H). MS (ESI) [M+H]⁺=245.6.

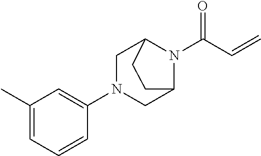 [SMILES: C=CC(=O)N1C2CCC1CN(c1cccc(C)c1)C2]

Example 12: 1-(3-(m-tolyl)-3,8-diazabicyclo[3.2.1]octan-8-yl)prop-2-en-1-one(XS165-33). Example 12 was synthesized following similar procedure for preparing example 5. White solid, 13% yield. ¹H NMR (400 MHz, DMSO-d₆) δ 7.05 (t, J=7.8 Hz, 1H), 6.80-6.70 (m, 1H), 6.69-6.60 (m, 2H), 6.56 (d, J=7.5 Hz, 1H), 6.17 (dt, J=16.5, 2.3 Hz, 1H), 5.69 (dt, J=10.2, 2.3 Hz, 1H), 4.73-4.50 (m, 2H), 3.52 (t, J=13.1 Hz, 2H), 2.71 (dd, J=25.4, 11.2 Hz, 2H), 2.54-2.45 (m, 4H), 2.22 (s, 3H), 1.88 (dd, J=17.7, 7.5 Hz, 2H), 1.79 (d, J=8.3 Hz, 2H). MS (ESI) [M+H]⁻=257.5.

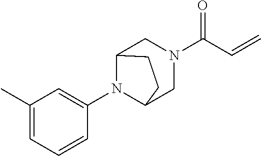 [SMILES: C=CC(=O)N1CC2CCC(C1)N2c1cccc(C)c1]

Example 13: 1-(8-(m-tolyl)-3,8-diazabicyclo[3.2.1]octan-3-yl)prop-2-en-1-one (XS165-54). Example 13 was synthesized following similar procedure for preparing example 5. White solid, 17% yield. ¹H NMR (400 MHz, Methanol-d₄) δ 7.15-7.06 (m, 1H), 6.82 (s, 1H), 6.77 (d, J=8.3 Hz, 1H), 6.69-6.55 (m, 2H), 6.15 (dt, J=16.8, 2.0 Hz, 1H), 5.68 (dd, J=10.6, 2.0 Hz, 1H), 4.31 (d, J=5.3 Hz, 2H), 4.21 (d, J=13.5 Hz, 1H), 3.75 (d, J=13.0 Hz, 1H), 3.53 (d, J=13.1 Hz, 1H), 3.10 (d, J=13.4 Hz, 1H), 2.25 (s, 3H), 1.96 (t, J=6.3 Hz, 2H), 1.77-1.63 (m, 2H). MS (ESI) [M+H]⁺=257.4.

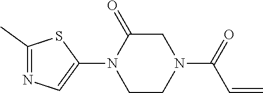 [SMILES: C=CC(=O)N1CCN(c2cnc(C)s2)C(=O)C1]

Example 14: 4-acryloyl-1-(2-methylthiazol-5-yl)piperazin-2-one (XS154-184). Example 13 was synthesized following similar procedure for preparing example 1. White solid, 43% yield. ¹H NMR (400 MHz, Methanol-d₄) δ 7.66 (d, J=44.8 Hz, 1H), 6.94-6.68 (m, 1H), 6.32 (d, J=16.6 Hz, 1H), 5.85 (d, J=9.2 Hz, 1H), 4.74-4.48 (m, 3H), 4.16-3.93 (m, 4H), 2.67 (s, 3H). MS (ESI) [M+H]⁺=252.0

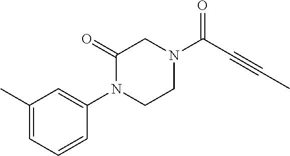 [SMILES: CC#CC(=O)N1CCN(c2cccc(C)c2)C(=O)C1]

Example 15: 4-(but-2-ynoyl)-1-(m-tolyl)piperazin-2-one (XS165-75). Example 15 was synthesized following similar procedure for preparing example 1. Colorless oil, 19% yield. ¹H NMR (400 MHz, Methanol-d4) δ 7.36-7.25 (m, 1H), 7.20-7.03 (m, 3H), 4.54 (d, J=1.9 Hz, 1H), 4.32 (d, J=1.8 Hz, 1H), 4.16 (td, J=5.6, 1.9 Hz, 1H), 4.00-3.90 (m, 1H), 3.83-3.79 (m, 1H), 3.78-3.72 (m, 1H), 2.36 (d, J=1.8 Hz, 3H), 2.07 (dd, J=8.9, 1.8 Hz, 3H). MS (ESI) [M+H]⁺=252.5.

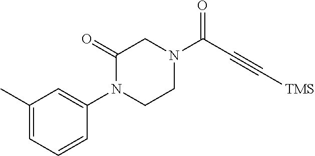 [SMILES: Cc1cccc(N2CCN(C(=O)C#C[Si](C)(C)C)CC2=O)c1]

Example 16: 1-(m-tolyl)-4-(3-(trimethylsilyl)propioloyl)piperazin-2-one (XS165-77). Example 16 was synthesized following similar procedure for preparing example 1. Colorless oil, 14% yield. ¹H NMR (400 MHz, DMSO-d₆) δ 7.29 (t, J=7.7 Hz, 1H), 7.12 (dd, J=22.6, 9.5 Hz, 3H), 4.42 (s, 1H), 4.17 (s, 1H), 4.03 (t, J=5.4 Hz, 1H), 3.84-3.73 (m, 2H), 3.68 (t, J=5.5 Hz, 1H), 2.31 (s, 3H), 0.25 (d, J=3.5 Hz, 9H). MS (ESI) [M+H]⁺=243.5.

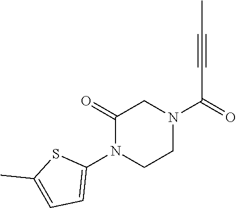 [SMILES: CC#CC(=O)N1CCN(c2ccc(C)s2)C(=O)C1]

Example 17: 4-(but-2-ynoyl)-1-(5-methylthiophen-2-yl)piperazin-2-one (XS165-127). Example 17 was synthesized following similar procedure for preparing example 1. Colorless oil, 14% yield. ¹H NMR (400 MHz, Chloroform-d) δ 6.55 (q, J=3.6, 3.2 Hz, 1H), 6.47 (dt, J=8.8, 2.8 Hz, 1H), 4.59 (d, J=2.1 Hz, 1H), 4.43 (d, J=2.0 Hz, 1H), 4.18-4.07 (m, 1H), 4.04-3.93 (m, 1H), 3.91-3.84 (m, 1H), 3.84-3.75 (m, 1H), 2.42 (d, J=2.4 Hz, 3H), 2.04 (dd, J=5.0, 2.1 Hz, 3H). MS (ESI) [M+H]⁺=263.1.

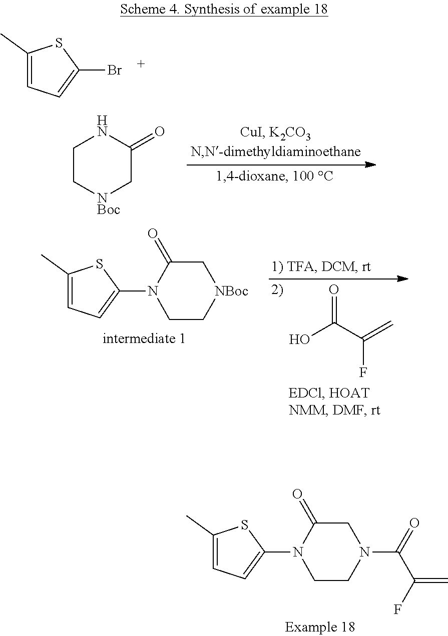

Example 18: 4-(2-fluoroacryloyl)-1-(5-methylthiophen-2-yl)piperazin-2-one (XS165-106). To a solution of 2-bromo-5-methylthiophene (50.0 mg, 0.28 mmol) dissolved in dioxane (1 mL), N,N′-dimethylethylenediamine (9 μL, 0.0846 mmol, 0.3 eq), K₂CO₃ (116.9 mg, 0.846 mmol, 3.0 eq), CuI (5.4 mg, 0.028 mmol, 0.1 eq) followed by tert-butyl 3-oxopiperazine-1-carboxylate (84.7 mg, 0.42 mmol, 1.5 eq). The reaction mixture was stirred at 100° C. under nitrogen atmosphere overnight. After cooling down to rt, resulting crude mixtures were purified via silica gel column chromatography to yield intermediate 1.

To a solution of intermediate 1 (40 mg, 0.135 mmol) dissolved in DCM (0.5 mL), and the TFA(0.5 mL) were added. The reaction mixture stirred at rt 1 h. Excess TFA was removed, resulting crude product was re-dissovled in DMF (1 mL), EDCI (58.8 mg, 0.306 mmol, 2.0 eq) and HOAT (41.7 mg, 0.306 mmol, 2.0 eq) were added. At rt the 2-fluoroacrylic acid (13.8 mg, 0.153 mmol, 1.0 eq.) was added, the reaction mixture stirred at rt for 2 h. Resulting crude mixtures were purified via prep-HPLC to yield title compound (White solid, 39% yield)¹H NMR (400 MHz, Methanol-d₄) δ 6.67-6.62 (m, 1H), 6.60 (d, J=4.0 Hz, 1H), 5.50-5.35 (m, 1H), 5.35-5.29 (m, 1H), 4.44 (s, 2H), 4.05 (t, J=5.5 Hz, 2H), 3.93 (t, J=5.5 Hz, 2H), 2.41 (s, 3H). MS (ESI) [2(M−1)+H]⁺=535.4.

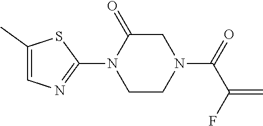 [SMILES: C=C(F)C(=O)N1CCN(c2ncc(C)s2)C(=O)C1]

Example 19: 4-(2-fluoroacryloyl)-1-(5-methylthiazol-2-yl)piperazin-2-one (XS165-112). Example 19 was synthesized following similar procedure for preparing example 18. White solid, 40% yield. ¹H NMR (400 MHz, Methanol-d₄) δ 7.15 (s, 1H), 5.44-5.29 (m, 1H), 5.29-5.24 (m, 1H), 4.47 (s, 2H), 4.19 (d, J=5.7 Hz, 2H), 4.00 (t, J=5.7 Hz, 2H), 2.36 (d, J=2.0 Hz, 3H). MS (ESI) [M+H]⁺=309.1.

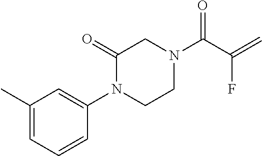 [SMILES: C=C(F)C(=O)N1CCN(c2cccc(C)c2)C(=O)C1]

Example 20: 4-(2-fluoroacryloyl)-1-(m-tolyl)piperazin-2-one (XS165-97). Example 20 was synthesized following similar procedure for preparing example 18. White solid, 56% yield. ¹H NMR (400 MHz, Chloroform-d) δ 7.34-7.28 (m, 1H), 7.15-7.09 (m, 2H), 7.06 (d, J=8.0 Hz, 1H), 5.57-5.35 (m, 1H), 5.34-5.14 (m, 1H), 4.44 (s, 2H), 3.98 (t, J=5.7 Hz, 2H), 3.78 (t, J=4.8 Hz, 2H), 2.37 (s, 3H). MS (ESI) [M+H]⁺=263.7.

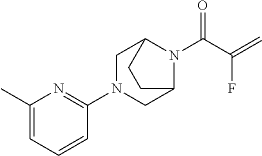 [SMILES: C=C(F)C(=O)N1C2CCC1CN(c1cccc(C)n1)C2]

Example 21: 2-fluoro-1-(3-(6-methylpyridin-2-yl)-3,8-diazabicyclo[3.2.1]octan-8-yl)prop-2-en-1-one (XS165-118). Example 21 was synthesized following similar procedure for preparing example 18. White solid, 45% yield. ¹H NMR (400 MHz, Methanol-d4) δ 8.01-7.87 (m, 1H), 7.17 (d, J=9.2 Hz, 1H), 6.90 (d, J=7.3 Hz, 1H), 5.49 (dt, J=47.5, 2.9 Hz, 1H), 5.31 (dt, J=16.9, 2.9 Hz, 1H), 4.87 (s, 16H), 4.02 (d, J=12.1 Hz, 2H), 3.51-3.39 (m, 2H), 2.58 (d, J=2.1 Hz, 3H), 2.08 (s, 2H), 1.93 (d, J=8.7 Hz, 2H). MS (ESI) [M+H]⁺=276.2.

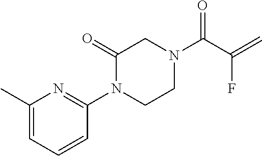 [SMILES: C=C(F)C(=O)N1CCN(c2cccc(C)n2)C(=O)C1]

Example 22: 4-(2-fluoroacryloyl)-1-(6-methylpyridin-2-yl)piperazin-2-one (XS165-110). Example 22 was synthesized following similar procedure for preparing example 18. White solid, 49% yield. ¹H NMR (400 MHz, Methanol-d₄) δ 7.80 (td, J=8.0, 1.8 Hz, 1H), 7.59 (d, J=8.1 Hz, 1H), 7.20 (dd, J=7.6, 2.1 Hz, 1H), 5.51-5.25 (m, 2H), 4.42 (s, 2H), 4.16-4.05 (m, 2H), 3.99 (s, 2H), 2.52 (d, J=2.1 Hz, 3H). MS (ESI) [M+H]⁺=264.1.

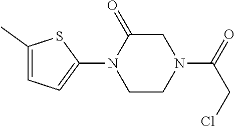 [SMILES: Cc1ccc(N2CCN(C(=O)CCl)CC2=O)s1]

Example 23: 4-(2-chloroacetyl)-1-(5-methylthiophen-2-yl)piperazin-2-one (XS165-119). Example 23 was synthesized following similar procedure for preparing example 1. White solid, 32% yield. ¹H NMR (400 MHz, Methanol-d₄) δ 6.64 (d, J=3.9 Hz, 1H), 6.60 (d, J=3.9 Hz, 1H), 4.48-4.30 (m, 4H), 4.08-3.84 (m, 4H), 2.41 (s, 3H). MS (ESI) [M+H]⁺=273.1.

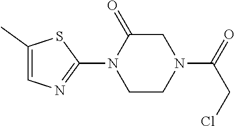 [SMILES: Cc1cnc(N2CCN(C(=O)CCl)CC2=O)s1]

Example 24: 4-(2-chloroacetyl)-1-(5-methylthiazol-2-yl)piperazin-2-one (XS165-123) Example 24 was synthesized following similar procedure for preparing example 1. White solid, 26% yield. ¹H NMR (400 MHz, Chloroform-d) δ 7.16 (s, 1H), 4.48 (d, J=6.3 Hz, 2H), 4.34 (d, J=5.7 Hz, 1H), 4.24 (d, J=5.4 Hz, 1H), 4.12 (d, J=8.6 Hz, 2H), 4.04-3.88 (m, 2H), 2.41 (s, 3H). MS (ESI) [M+H]⁺=273.4.

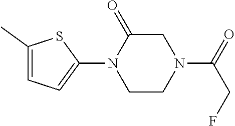 [SMILES: Cc1ccc(N2CCN(C(=O)CF)CC2=O)s1]

Example 25: 4-(2-fluoroacetyl)-1-(5-methylthiophen-2-yl)piperazin-2-one (XS165-126). Example 25 was synthesized following similar procedure for preparing example 18. White solid, 40% yield. ¹H NMR (400 MHz, Chloroform-d) δ 6.56 (d, J=3.7 Hz, 1H), 6.49 (d, J=5.5 Hz, 1H), 5.10 (d, J=10.6 Hz, 1H), 4.99 (d, J=10.2 Hz, 1H), 4.40 (d, J=34.5 Hz, 2H), 4.09-3.77 (m, 4H), 2.42 (d, J=2.1 Hz, 3H). MS (ESI) [M+H]⁺=257.1.

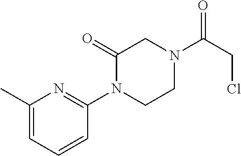 [SMILES: Cc1cccc(N2CCN(C(=O)CCl)CC2=O)n1]

Example 26: 4-(2-chloroacetyl)-1-(6-methylpyridin-2-yl)piperazin-2-one (XS165-120). Example 26 was synthesized following similar procedure for preparing example 18. White solid, 35% yield. ¹H NMR (400 MHz, Chloroform-d) δ 7.72 (dd, J=23.9, 8.2 Hz, 1H), 7.61 (t, J=7.9 Hz, 1H), 6.99 (t, J=6.7 Hz, 1H), 4.40 (d, J=9.7 Hz, 2H), 4.30-4.23 (m, 1H), 4.17 (d, J=5.7 Hz, 1H), 4.12 (d, J=2.9 Hz, 2H), 3.97-3.84 (m, 2H), 2.50 (s, 3H). MS (ESI) [M+H]⁺=268.1.

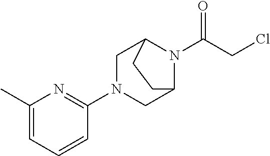 [SMILES: Cc1cccc(N2CC3CCC(C2)N3C(=O)CCl)n1]

Example 27: 2-chloro-1-(3-(6-methylpyridin-2-yl)-3,8-diazabicyclo[3.2.1]octan-8-yl)ethan-1-one (XS165-121). Example 27 was synthesized following similar procedure for preparing example 18. White solid, 47% yield. ¹H NMR (400 MHz, Chloroform-d) δ 7.44-7.30 (m, 1H), 6.52 (d, J=7.3 Hz, 1H), 6.35 (d, J=8.5 Hz, 1H), 4.82 (d, J=6.4 Hz, 1H), 4.37 (d, J=6.5 Hz, 1H), 4.20 (d, J=12.2 Hz, 1H), 4.13-4.01 (m, 2H), 3.92 (d, J=12.0 Hz, 1H), 3.08 (dd, J=12.1, 2.9 Hz, 2H), 2.39 (s, 3H), 2.10-1.77 (m, 4H). MS (ESI) [M+H]⁺=280.1.

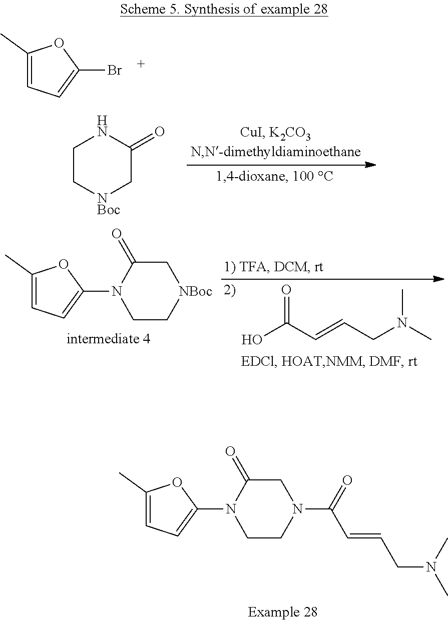

Example 28: (E)-4-(4-(dimethylamino)but-2-enoyl)-1-(5-methylfuran-2-yl)piperazin-2-one (XS165-100). To a solution of 2-bromo-5-methylfuran(50.0 mg, 0.28 mmol) dissolved in dioxane (1 mL), N,N′-dimethylethylenediamine (9 μL, 0.0846 mmol, 0.3 eq), K₂CO₃ (116.9 mg, 0.846 mmol, 3.0 eq), CuI (5.4 mg, 0.028 mmol, 0.1 eq) followed by tert-butyl 3-oxopiperazine-1-carboxylate (84.7 mg, 0.42 mmol, 1.5 eq). The reaction mixture was stirred at 100° C. under nitrogen atmosphere overnight. After cooling down to rt, resulting crude mixtures were purified via silica gel column chromatography to yield intermediate 5.

To a solution of intermediate 1 (30 mg, 0.107 mmol) dissolved in DCM (0.5 mL), and the TFA(0.5 mL) were added. The reaction mixture stirred at rt 1 h. Excess TFA was removed, resulting crude product was re-dissovled in DMF (1 mL), EDCI (41.1 mg, 0.214 mmol, 2.0 eq) and HOAT (29.2 mg, 0.214 mmol, 2.0 eq) were added. At rt the (E)-4-(dimethylamino)but-2-enoic acid (17.7 mg, 0.107 mmol, 1.0 eq.) was added, the reaction mixture stirred at rt for 2 h. Resulting crude mixtures were purified via prep-HPLC to yield title compound (White solid, 31% yield) ¹H NMR (400 MHz, Methanol-d₄) δ 7.02-6.87 (m, 1H), 6.78 (q, J=9.6, 8.5 Hz, 1H), 6.22 (t, J=2.7 Hz, 1H), 6.08-6.00 (m, 1H), 4.44 (d, J=28.2 Hz, 2H), 4.05-3.96 (m, 4H), 3.93-3.83 (m, 2H), 2.93 (d, J=2.1 Hz, 6H), 2.26 (d, J=1.8 Hz, 3H). MS (ESI) [M+H]⁺=292.2.

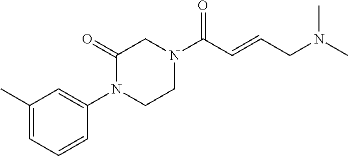

Example 29: (E)-4-(4-(dimethylamino)but-2-enoyl)-1-(n-tolyl)piperazin-2-one (XS165-99). Example 29 was synthesized following similar procedure for preparing example 28. White solid, 54% yield. ¹H NMR (400 MHz, Methanol-d₄) δ 7.36-7.24 (m, 1H), 7.12 (d, J=5.7 Hz, 2H), 7.08 (d, J=8.0 Hz, 1H), 6.92 (dd, J=22.6, 15.1 Hz, 1H), 6.75 (dt, J=14.8, 7.1 Hz, 1H), 4.40 (d, J=26.9 Hz, 2H), 4.00 (p, J=5.2 Hz, 2H), 3.94 (d, J=7.2 Hz, 2H), 3.88-3.72 (m, 2H), 2.88 (d, J=1.8 Hz, 6H), 2.33 (d, J=1.8 Hz, 3H). MS (ESI) [M+H]⁺=302.1.

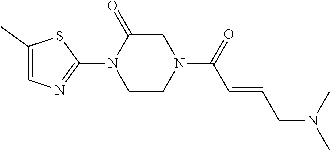

Example 30: (E)-4-(4-(dimethylamino)but-2-enoyl)-1-(5-methylthiazol-2-yl)piperazin-2-one (XS165-113). Example 30 was synthesized following similar procedure for preparing example 28. White solid, 42% yield. ¹H NMR (400 MHz, Methanol-d₄) δ 7.09 (s, 1H), 6.87 (dd, J=35.8, 15.1 Hz, 1H), 6.74-6.60 (m, 1H), 4.46 (d, J=35.7 Hz, 2H), 4.23-4.08 (m, 2H), 4.05-3.93 (m, 2H), 3.89 (d, J=7.1 Hz, 2H), 2.83 (s, 6H), 2.30 (d, J=2.0 Hz, 3H). MS (ESI) [M+H]⁺=270.1.

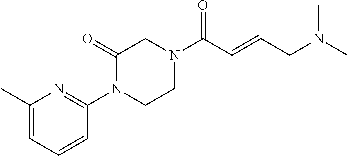

Example 31: (E)-4-(4-(dimethylamino)but-2-enoyl)-1-(6-methylpyridin-2-yl)piperazin-2-one (XS165-109). Example 31 was synthesized following similar procedure for preparing example 28. White solid, 47% yield. ¹H NMR (400 MHz, Methanol-d₄) δ 7.74 (t, J=7.9 Hz, 1H), 7.59 (t, J=9.7 Hz, 1H), 7.16 (d, J=7.5 Hz, 1H), 6.95 (t, J=16.4 Hz, 1H), 6.85-6.72 (m, 1H), 4.48 (d, J=27.1 Hz, 2H), 4.16 (d, J=5.6 Hz, 1H), 4.09 (t, J=5.0 Hz, 1H), 4.06-3.93 (m, 4H), 2.93 (d, J=2.0 Hz, 6H), 2.52 (d, J=2.1 Hz, 3H). MS (ESI) [M+H]⁺=303.2.

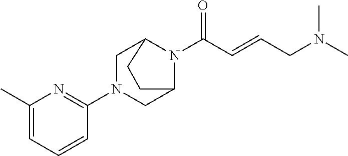

Example 32: (E)-4-(dimethylamino)-1-(3-(6-methylpyridin-2-yl)-3,8-diazabicyclo[3.2.1]octan-8-yl)but-2-en-1-one (XS165-117). Example 32 was synthesized following similar procedure for preparing example 5. White solid, 23% yield. ¹H NMR (400 MHz, Methanol-d₄) δ 7.92 (ddd, J=9.2, 7.0, 1.9 Hz, 1H), 7.14 (d, J=9.2 Hz, 1H), 6.95-6.85 (m, 2H), 6.85-6.74 (m, 1H), 4.09-3.87 (m, 4H), 3.38 (t, J=13.2 Hz, 2H), 2.88 (d, J=1.9 Hz, 6H), 2.55 (d, J=2.0 Hz, 3H), 2.12 (dt, J=16.7, 8.2 Hz, 1H), 2.06-1.80 (m, 3H). MS (ESI) [M+H]⁺=315.2.

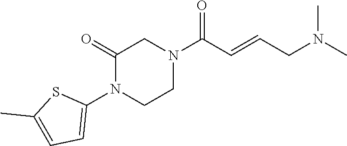

Example 33: (E)-4-(4-(dimethylamino)but-2-enoyl)-1-(5-methylthiophen-2-yl)piperazin-2-one (XS165-105). Example 33 was synthesized following similar procedure for preparing example 28. White solid, 56% yield. ¹H NMR (400 MHz, Methanol-d₄) δ 6.92 (dd, J=35.8, 15.2 Hz, 1H), 6.75 (dt, J=14.9, 7.2 Hz, 1H), 6.62-6.48 (m, 2H), 4.43 (d, J=29.9 Hz, 2H), 4.09-3.97 (m, 2H), 3.99-3.77 (m, 4H), 2.89 (d, J=2.1 Hz, 6H), 2.36 (d, J=2.1 Hz, 3H). MS (ESI) [M+H]⁺=308.4.

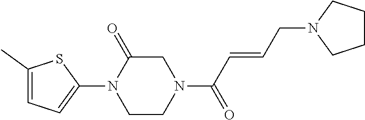 [SMILES: Cc1ccc(N2CCN(C(=O)/C=C/CN3CCCC3)CC2=O)s1]

Example 34: (E)-1-(5-methylthiophen-2-yl)-4-(4-(pyrrolidin-1-yl)but-2-enoyl)piperazin-2-one (XS165-154). Example 34 was synthesized following similar procedure for preparing example 28. White solid, 58% yield. ¹H NMR (400 MHz, Methanol-d4) δ 6.90 (dd, J=36.2, 15.2 Hz, 1H), 6.81-6.68 (m, 1H), 6.65-6.44 (m, 2H), 4.43 (d, J=30.7 Hz, 2H), 4.00 (d, J=7.1 Hz, 4H), 3.94-3.78 (m, 2H), 3.62 (s, 2H), 3.12 (s, 2H), 2.37 (d, J=2.3 Hz, 3H), 2.08 (d, J=48.5 Hz, 4H). MS (ESI) [M+H]⁺=334.2.

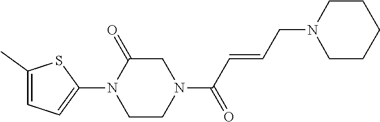 [SMILES: Cc1ccc(N2CCN(C(=O)/C=C/CN3CCCCC3)CC2=O)s1]

Example 35: (E)-1-(5-methylthiophen-2-yl)-4-(4-(piperidin-1-yl)but-2-enoyl)piperazin-2-one (XS165-170). Example 35 was synthesized following similar procedure for preparing example 28. White solid, 55% yield. ¹H NMR (400 MHz, Methanol-d₄) δ 6.91 (dd, J=34.6, 15.2 Hz, 1H), 6.82-6.67 (m, 1H), 6.57 (dt, J=11.4, 3.5 Hz, 2H), 4.43 (d, J=29.1 Hz, 2H), 4.02 (q, J=8.4, 6.8 Hz, 2H), 3.88 (dd, J=25.4, 6.4 Hz, 4H), 3.51 (d, J=12.4 Hz, 2H), 2.94 (t, J=12.5 Hz, 2H), 2.37 (d, J=2.3 Hz, 3H), 1.95 (d, J=14.4 Hz, 2H), 1.88-1.63 (m, 3H), 1.59-1.40 (m, 1H). MS (ESI) [M+H]⁺=348.3.

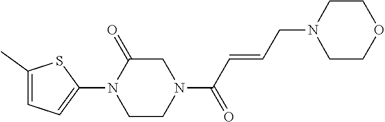 [SMILES: Cc1ccc(N2CCN(C(=O)/C=C/CN3CCOCC3)CC2=O)s1]

Example 36: (E)-1-(5-methylthiophen-2-yl)-4-(4-morpholinobut-2-enoyl)piperazin-2-one (XS165-155). Example 36 was synthesized following similar procedure for preparing example 28. White solid, 48% yield. ¹H NMR (400 MHz, Methanol-d₄) δ 6.95 (dd, J=34.0, 15.1 Hz, 1H), 6.84-6.68 (m, 1H), 6.65-6.45 (m, 2H), 4.44 (d, J=30.6 Hz, 2H), 4.13-3.63 (m, 10H), 3.44 (s, 2H), 3.20 (s, 2H), 2.37 (s, 3H). MS (ESI) [M+H]⁺=350.2.

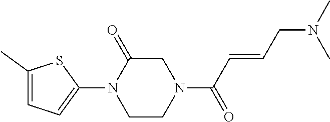

Example 37: (E)-4-(dimethylamino)-1-(4-(5-methylthiophen-2-yl)piperazin-1-yl)but-2-en-1-one (XS165-172). Example 37 was synthesized following similar procedure for preparing example 28. White solid, 33% yield. ¹H NMR (400 MHz, Methanol-d₄) δ 7.00-6.72 (m, 2H), 6.73-6.30 (m, 2H), 4.12-3.84 (m, 8H), 3.76-3.69 (m, 2H), 2.89-2.85 (m, 9H). MS (ESI) [M+H]⁺=294.4.

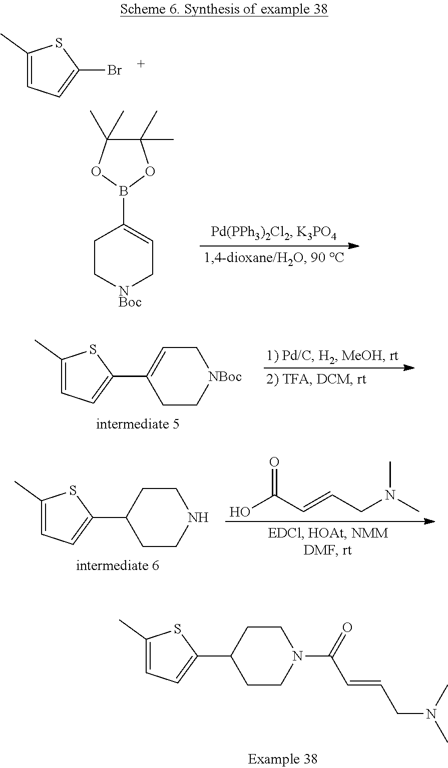

Example 38: (E)-4-(dimethylamino)-1-(4-(5-methylthiophen-2-yl)piperidin-1-yl)but-2-en-1-one (XS165-169). ). To a solution of the 2-bromo-5-methylthiophene (177.1 mg, 1 mmol) dissolved in dioxane/H₂O=4:1 (3 mL), K₃PO₄ (424.6 mg, 2 mmol, 2.0 eq), Pd(PPh₃)₂Cl₂ (70.2 mg, 0.1 mmol, 0.1 eq), followed by tert-butyl 4-(4,4,5,5-tetramethyl-1,3,2-dioxaborolan-2-yl)-3,6-dihydropyridine-1(2H)-carboxylate (618.4 mg, 2.0 mmol, 2.0 eq). The reaction mixture was stirred at 90° C. under nitrogen atmosphere overnight. After cooling down to rt, resulting crude mixtures were purified via silica gel column chromatography to yield intermediate 5.

To a solution of intermediate 5 (200 mg, 0.72 mmol) dissolved in MeOH (3 mL), and the Pd/C(20 mg) were added. The reaction mixture stirred at hydrogen atmosphere. MeOH was removed, resulting crude product was re-dissovled in DCM (1 mL), TFA (1 mL) was added. The reaction mixture stirred at rt for 1 h. Excess TFA was removed, resulting crude product was re-dissovled in DMF (1 mL), EDCI (272 mg, 1.42 mmol, 2.0 eq) and HOAT (187.4 mg, 1.42 mmol, 2.0 eq) were added. At rt the (E)-4-(dimethylamino)but-2-enoic acid (117.6 mg, 0.71 mmol, 1.0 eq.) was added, the reaction mixture stirred at rt for 2 h. Resulting crude mixtures were purified via prep-HPLC to yield title compound (White solid, 43% yield)¹H NMR (400 MHz, Methanol-d₄) δ 6.95 (d, J=15.1 Hz, 1H), 6.72-6.58 (m, 2H), 6.55 (d, J=2.8 Hz, 1H), 4.59 (d, J=13.4 Hz, 1H), 4.13 (d, J=13.9 Hz, 1H), 3.92 (dd, J=7.3, 2.2 Hz, 2H), 3.24 (d, J=13.1 Hz, 1H), 3.14-3.00 (m, 1H), 2.95-2.76 (m, 7H), 2.38 (d, J=2.3 Hz, 3H), 2.13-1.96 (m, 2H), 1.74-1.42 (m, 2H). MS (ESI) [M+H]⁺=293.4.

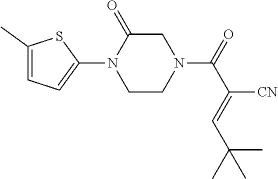 [SMILES: Cc1ccc(N2CCN(C(=O)/C(C#N)=C/C(C)(C)C)CC2=O)s1]

Example 39: (E)-4,4-dimethyl-2-(4-(5-methylthiophen-2-yl)-3-oxopiperazine-1-carbonyl)pent-2-enenitrile (XS165-177). Example 39 was synthesized following similar procedure for preparing example 28. White solid, 47% yield. ¹H NMR (400 MHz, Methanol-d₄) δ 7.12-6.97 (m, 1H), 6.67-6.43 (m, 2H), 4.38 (s, 2H), 4.08-3.76 (m, 4H), 2.39 (s, 3H), 1.32 (s, 9H). MS (ESI) [M+H]⁺=332.2.

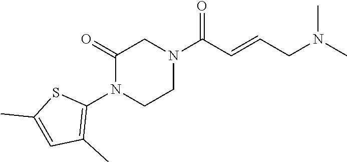

Example 40: (E)-4-(4-(dimethylamino)but-2-enoyl)-1-(3,5-dimethylthiophen-2-yl)piperazin-2-one (XS175-45). Example 40 was synthesized following similar procedure for preparing example 28. White solid, 49% yield. ¹H NMR (400 MHz, Methanol-d₄) δ 6.75 (dd, J=24.0, 15.0 Hz, 1H), 6.65-6.49 (m, 1H), 6.32 (d, J=2.8 Hz, 1H), 4.36-4.11 (m, 2H), 3.84 (d, J=7.1 Hz, 2H), 3.79-3.69 (m, 2H), 3.62-3.42 (m, 2H), 2.71 (s, 6H), 2.19 (s, 3H), 1.78 (s, 3H). MS (ESI) [M+H]⁺=322.2.

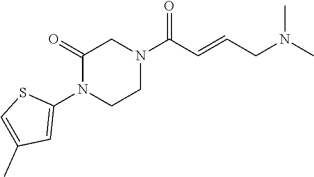 [SMILES: Cc1csc(N2CCN(C(=O)/C=C/CN(C)C)CC2=O)c1]

Example 41: (E)-4-(4-(dimethylamino)but-2-enoyl)-1-(4-methylthiophen-2-yl)piperazin-2-one (XS175-46). Example 41 was synthesized following similar procedure for preparing example 28. White solid, 59% yield. ¹H NMR (400 MHz, Methanol-d₄) δ 6.91 (dd, J=37.4, 15.2 Hz, 1H), 6.79-6.67 (m, 1H), 6.60 (dd, J=5.2, 2.7 Hz, 2H), 4.42 (d, J=30.8 Hz, 2H), 4.02 (d, J=7.6 Hz, 2H), 3.97-3.77 (m, 4H), 2.88 (s, 6H), 2.17 (s, 3H). MS (ESI) [M+H]⁺=308.3.

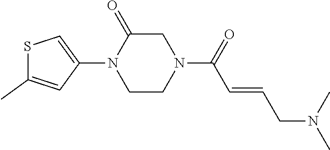

Example 42: (E)-4-(4-(dimethylamino)but-2-enoyl)-1-(5-methylthiophen-3-yl)piperazin-2-one (XS175-59). Example 42 was synthesized following similar procedure for preparing example 28. White solid, 53% yield. ¹H NMR (400 MHz, Methanol-d₄) δ 7.12 (s, 1H), 6.90 (d, J=36.9 Hz, 2H), 6.80-6.64 (m, 1H), 4.49-4.27 (m, 2H), 4.09-3.91 (m, 4H), 3.91-3.72 (m, 2H), 2.89 (s, 6H), 2.42 (s, 3H). MS (ESI) [M+H]⁺=309.1.

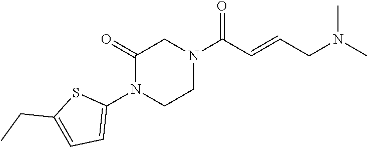 [SMILES: CCc1ccc(N2CCN(C(=O)/C=C/CN(C)C)CC2=O)s1]

Example 43: (E)-4-(4-(dimethylamino)but-2-enoyl)-1-(5-ethylthiophen-2-yl)piperazin-2-one (XS175-63). Example 43 was synthesized following similar procedure for preparing example 28. White solid, 49% yield. ¹H NMR (400 MHz, Methanol-d₄) δ 6.92 (dd, J=35.5, 15.2 Hz, 1H), 6.81-6.68 (m, 1H), 6.59 (q, J=3.5 Hz, 2H), 4.43 (d, J=29.9 Hz, 2H), 4.02 (s, 2H), 3.97-3.78 (m, 4H), 2.88 (s, 6H), 2.73 (q, J=7.2 Hz, 2H), 1.24 (t, J=6.9 Hz, 3H). MS (ESI) [M+H]⁺=322.2.

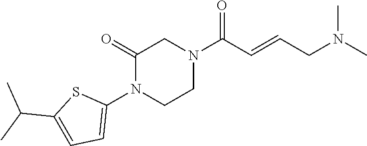 [SMILES: CC(C)c1ccc(N2CCN(C(=O)/C=C/CN(C)C)CC2=O)s1]

Example 44: (E)-4-(4-(dimethylamino)but-2-enoyl)-1-(5-isopropylthiophen-2-yl)piperazin-2-one (XS175-64). Example 44 was synthesized following similar procedure for preparing example 28. White solid, 39% yield. ¹H NMR (400 MHz, Methanol-d₄) δ 6.94 (dd, J=35.2, 15.2 Hz, 1H), 6.82-6.69 (m, 1H), 6.62 (s, 2H), 4.56-4.33 (m, 2H), 4.03 (d, J=8.5 Hz, 2H), 3.99-3.82 (m, 4H), 3.13-3.00 (m, 1H), 2.90 (s, 6H), 1.29 (d, J=6.6 Hz, 6H). MS (ESI) [M+H]⁺=336.2.

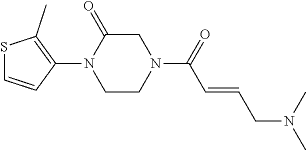 [SMILES: Cc1sccc1N1CCN(C(=O)/C=C/CN(C)C)CC1=O]

Example 45: (E)-4-(4-(dimethylamino)but-2-enoyl)-1-(2-methylthiophen-3-yl)piperazin-2-one (XS175-67). Example 45 was synthesized following similar procedure for preparing example 28. White solid, 49% yield. ¹H NMR (400 MHz, Methanol-d4) δ 7.19 (dd, J=6.1, 2.7 Hz, 1H), 7.02-6.83 (m, 2H), 6.82-6.66 (m, 1H), 4.54-4.33 (m, 2H), 4.01 (d, J=7.0 Hz, 2H), 3.98-3.87 (m, 2H), 3.78-3.57 (m, 2H), 2.89 (s, 6H), 2.26 (s, 3H). MS (ESI) [M+H]⁺=308.3.

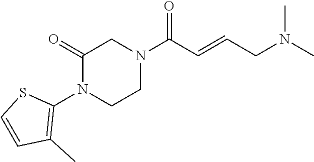 [SMILES: Cc1ccsc1N1CCN(C(=O)/C=C/CN(C)C)CC1=O]

Example 46: (E)-4-(4-(dimethylamino)but-2-enoyl)-1-(3-methylthiophen-2-yl)piperazin-2-one (XS175-68). Example 46 was synthesized following similar procedure for preparing example 28. White solid, 51% yield. ¹H NMR (400 MHz, Methanol-d4) δ 7.23 (d, J=5.3 Hz, 1H), 7.03-6.87 (m, 1H), 6.84 (d, J=5.4 Hz, 1H), 6.77 (dt, J=14.8, 7.2 Hz, 1H), 4.46 (d, J=30.5 Hz, 2H), 4.04 (q, J=5.8 Hz, 2H), 4.00-3.87 (m, 2H), 3.82-3.64 (m, 2H), 2.90 (s, 6H), 2.06 (s, 3H). MS (ESI) [M+H]⁺=308.2.

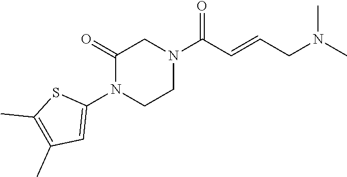

Example 47: (E)-4-(4-(dimethylamino)but-2-enoyl)-1-(4,5-dimethylthiophen-2-yl)piperazin-2-one (XS175-70). Example 47 was synthesized following similar procedure for preparing example 28. White solid, 53% yield. ¹H NMR (400 MHz, Methanol-d₄) δ 6.93 (dd, J=36.6, 15.2 Hz, 1H), 6.76 (dt, J=15.0, 7.4 Hz, 1H), 6.50 (s, 1H), 4.43 (d, J=29.5 Hz, 2H), 4.03 (s, 2H), 3.95 (d, J=7.3 Hz, 2H), 3.93-3.77 (m, 2H), 2.90 (s, 6H), 2.24 (s, 3H), 2.07 (s, 3H). MS (ESI) [M+H]⁻=322.2.

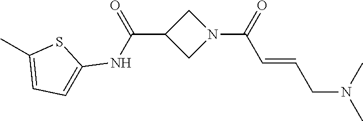

Example 48: (E)-1-(4-(dimethylamino)but-2-enoyl)-N-(5-methylthiophen-2-yl)azetidine-3-carboxamide (XS175-71). Example 48 was synthesized following similar procedure for preparing example 28. White solid, 24% yield. ¹H NMR (400 MHz, Methanol-d₄) δ 6.80-6.62 (m, 1H), 6.53-6.37 (m, 3H), 4.54-4.39 (m, 2H), 4.29-4.20 (m, 1H), 4.19-4.11 (m, 1H), 3.95-3.85 (m, 2H), 3.63-3.51 (m, 1H), 2.87 (s, 6H), 2.35 (s, 3H). MS (ESI) [M+H]⁺=308.3

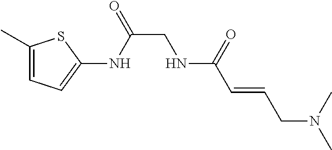

Example 49: (E)-4-(dimethylamino)-N-(2-((5-methylthiophen-2-yl)amino)-2-oxoethyl)but-2-enamide (XS175-76). Example 49 was synthesized following similar procedure for preparing example 28. White solid, 20% yield. ¹H NMR (400 MHz, Methanol-d₄) δ 6.74-6.56 (m, 1H), 6.46-6.32 (m, 3H), 4.04-3.95 (m, 2H), 3.90-3.81 (m, 2H), 2.81 (s, 6H), 2.28 (s, 3H). MS (ESI) [M+H]⁺=282.5.

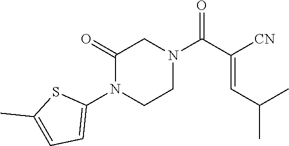 [SMILES: Cc1ccc(N2CCN(C(=O)/C(C#N)=C/C(C)C)CC2=O)s1]

Example 50: (E)-4-methyl-2-(4-(5-methylthiophen-2-yl)-3-oxopiperazine-1-carbonyl)pent-2-enenitrile (XS175-110). Example 50 was synthesized following similar procedure for preparing example 28. White solid, 57% yield. ¹H NMR (400 MHz, Methanol-d₄) δ 7.03 (d, J=10.1 Hz, 1H), 6.68-6.59 (m, 2H), 4.43 (s, 2H), 4.08-3.91 (m, 5H), 3.06-2.84 (m, 1H), 2.42 (s, 3H), 1.22-1.16 (m, 6H). MS (ESI) [M+H]⁺=248.3.

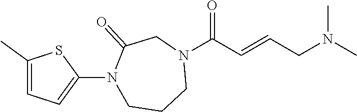

Example 51: (E)-4-(4-(dimethylamino)but-2-enoyl)-1-(5-methylthiophen-2-yl)-1,4-diazepan-2-one (XS175-120). Example 51 was synthesized following similar procedure for preparing example 28. White solid, 47% yield. ¹H NMR (400 MHz, Methanol-d₄) δ 7.04-6.88 (m, 1H), 6.83-6.64 (m, 1H), 6.63-6.50 (m, 2H), 4.62-4.47 (m, 2H), 4.18-4.01 (m, 2H), 4.00-3.81 (m, 4H), 2.89 (s, 6H), 2.38 (s, 3H), 2.12-1.87 (m, 2H). MS (ESI) [M+H]⁺=322.3.

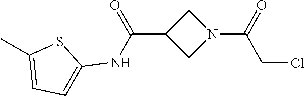 [SMILES: Cc1ccc(NC(=O)C2CN(C(=O)CCl)C2)s1]

Example 52: 1-(2-chloroacetyl)-N-(5-methylthiophen-2-yl)azetidine-3-carboxamide (XS175-126). Example 52 was synthesized following similar procedure for preparing example 28. White solid, 33% yield. ¹H NMR (400 MHz, Methanol-d₄) δ 6.56-6.37 (m, 2H), 4.55-4.40 (m, 2H), 4.29-4.12 (m, 2H), 4.09-4.00 (m, 2H), 3.66-3.53 (m, 1H), 2.38 (s, 3H). MS (ESI) [M+H]⁺=273.1.

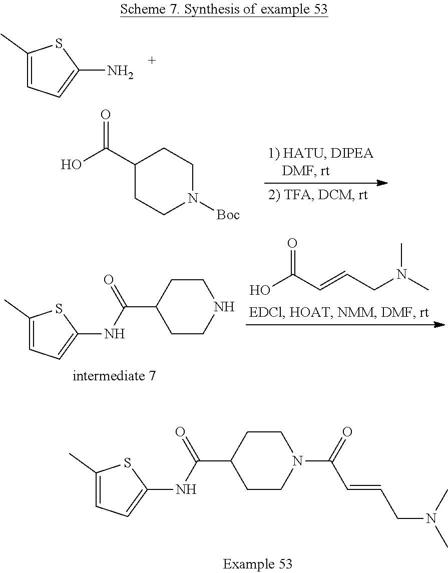

Example 53: (E)-1-(4-(dimethylamino)but-2-enoyl)-N-(5-methylthiophen-2-yl)piperidine-4-carboxamide (XS175-132). ). To a solution of the 5-methylthiophen-2-amine (113.2 mg, 1 mmol) dissolved in DMF (2 mL), HATU (418.2 mg, 1.1 mmol, 1.1 eq), DIPEA (0.18 mL, 2.0 mmol, 2.0 eq), followed by 1-(tert-butoxycarbonyl)piperidine-4-carboxylic acid (229.3 mg, 1.0 mmol, 1.0 eq). The reaction mixture was stirred at rt 3 h. The resulting crude mixtures were purified via silica gel column chromatography. The resulting crude product was re-dissovled in DCM (1 mL), TFA (1 mL) was added. The reaction mixture stirred at rt for 1 h. Excess TFA was removed, resulting crude product was were purified via silica gel column chromatography to yield intermediate 7.

To a solution of intermediate 7 (25 mg, 0.116 mmol) dissolved in DMF (1 mL), HATU (50.9 mg, 0.14 mmol, 1.2 eq), DIPEA (0.043 mL, 0.24 mmol, 2.0 eq), followed by (E)-4-(dimethylamino)but-2-enoic acid (18.5 mg, 0.116 mmol, 1.0 eq) the reaction mixture stirred at rt for 2 h. Resulting crude mixtures were purified via prep-HPLC to yield title compound (White solid, 43% yield)¹H NMR (400 MHz, Methanol-d₄) δ 6.88 (d, J=15.1 Hz, 1H), 6.65-6.50 (m, 1H), 6.41 (s, 2H), 4.49 (d, J=12.9 Hz, 1H), 4.07 (d, J=13.5 Hz, 1H), 3.85 (d, J=6.7 Hz, 2H), 3.16 (t, J=12.9 Hz, 1H), 2.82 (s, 7H), 2.59 (t, J=9.8 Hz, 1H), 2.28 (s, 3H), 1.84 (d, J=12.4 Hz, 2H), 1.61 (p, J=13.0 Hz, 2H). MS (ESI) [M+H]⁺=336.2.

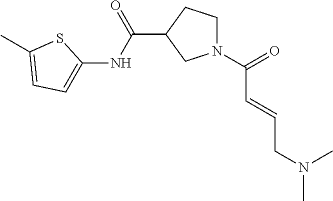

Example 54: (E)-1-(4-(dimethylamino)but-2-enoyl)-N-(5-methylthiophen-2-yl)pyrrolidine-3-carboxamide (XS175-133). Example 54 was synthesized following similar procedure for preparing example 53. White solid, 29% yield. ¹H NMR (400 MHz, Methanol-d₄) δ 6.73-6.56 (m, 2H), 6.40 (s, 2H), 3.85 (s, 2H), 3.83-3.37 (m, 4H), 3.20-3.04 (m, 1H), 2.80 (s, 6H), 2.27 (s, 3H), 2.25-1.94 (m, 2H). MS (ESI) [M+H]=322.

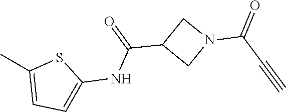 [SMILES: C#CC(=O)N1CC(C(=O)Nc2ccc(C)s2)C1]

Example 55: N-(5-methylthiophen-2-yl)-1-propioloylazetidine-3-carboxamide (XS175-137). Example 55 was synthesized following similar procedure for preparing example 53. White solid, 19% yield. ¹H NMR (400 MHz, Methanol-d₄) δ 6.50 (s, 2H), 4.42 (p, J=9.1, 8.3 Hz, 2H), 4.28-4.08 (m, 2H), 3.92 (s, 1H), 3.69-3.50 (m, 1H), 2.39 (s, 3H). MS (ESI) [M+H]⁺=321.1.

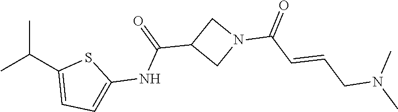 [SMILES: CC(C)c1ccc(NC(=O)C2CN(C(=O)/C=C/CN(C)C)C2)s1]

Example 56: (E)-1-(4-(dimethylamino)but-2-enoyl)-N-(5-isopropylthiophen-2-yl)azetidine-3-carboxamide (XS175-143). Example 56 was synthesized following similar procedure for preparing example 53. White solid, 37% yield. ¹H NMR (400 MHz, Methanol-d₄) δ 6.75-6.59 (m, 1H), 6.57-6.40 (m, 3H), 4.56-4.36 (m, 2H), 4.24 (t, J=9.4 Hz, 1H), 4.19-4.09 (m, 1H), 3.90 (d, J=6.7 Hz, 2H), 3.63-3.50 (m, 1H), 3.03 (h, J=6.5, 5.6 Hz, 1H), 2.86 (s, 6H), 1.25 (d, J=6.5 Hz, 6H). MS (ESI) [M+H]⁺=336.2.

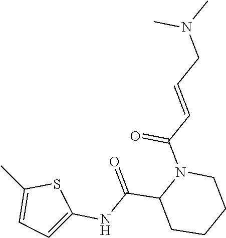 [SMILES: Cc1ccc(NC(=O)C2CCCCN2C(=O)/C=C/CN(C)C)s1]

Example 57: (E)-1-(4-(dimethylamino)but-2-enoyl)-N-(5-methylthiophen-2-yl)piperidine-2-carboxamide (XS175-148). Example 57 was synthesized following similar procedure for preparing example 53. White solid, 25% yield. ¹H NMR (400 MHz, Methanol-d₄) δ 7.07-6.79 (m, 1H), 6.72-6.43 (m, 3H), 5.23 (s, 1H), 4.10-3.82 (m, 3H), 3.61-3.44 (m, 1H), 2.89 (s, 6H), 2.36 (s, 3H), 2.29-2.14 (m, 1H), 1.87-1.64 (m, 3H), 1.63-1.41 (m, 2H). MS (ESI) [M+H]⁻=336.2.

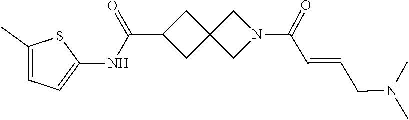

Example 58: (E)-2-(4-(dimethylamino)but-2-enoyl)-N-(5-methylthiophen-2-yl)-2-azaspiro[3.3]heptane-6-carboxamide (XS175-149). Example 58 was synthesized following similar procedure for preparing example 53. White solid, 16% yield. ¹H NMR (400 MHz, Methanol-d4) δ 6.70-6.53 (m, 1H), 6.49-6.28 (m, 3H), 4.39-4.18 (m, 2H), 4.09-3.95 (m, 2H), 3.94-3.80 (m, 2H), 3.13-2.97 (m, 1H), 2.84 (s, 6H), 2.44 (d, J=7.9 Hz, 4H), 2.31 (s, 3H). MS (ESI) [M+H]⁺=348.3.

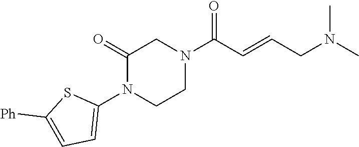 [SMILES: CN(C)C/C=C/C(=O)N1CCN(c2ccc(-c3ccccc3)s2)C(=O)C1]

Example 59: (E)-4-(4-(dimethylamino)but-2-enoyl)-1-(5-phenylthiophen-2-yl)piperazin-2-one (XS175-158). Example 59 was synthesized following similar procedure for preparing example 28. White solid, 50% yield. ¹H NMR (400 MHz, Methanol-d₄) δ 7.57 (d, J=7.5 Hz, 2H), 7.34 (t, J=7.1 Hz, 2H), 7.27-7.16 (m, 2H), 6.95 (dd, J=33.3, 14.9 Hz, 1H), 6.84-6.68 (m, 2H), 4.50 (d, J=30.7 Hz, 2H), 4.07 (s, 2H), 3.99 (s, 4H), 2.91 (s, 6H). MS (ESI) [M+H]=370.2.

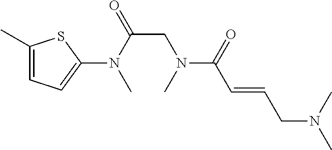

Example 60: (E)-4-(dimethylamino)-N-methyl-N-(2-(methyl(5-methylthiophen-2-yl)amino)-2-oxoethyl)but-2-enamide (XS175-159). Example 60 was synthesized following similar procedure for preparing example 28. White solid, 26% yield. ¹H NMR (400 MHz, Methanol-d₄) δ 6.93-6.75 (m, 2H), 6.69 (s, 1H), 6.51 (d, J=15.2 Hz, 1H), 4.14 (d, J=7.3 Hz, 2H), 4.03 (s, 2H), 3.26 (s, 3H), 3.16 (s, 9H), 2.48 (s, 3H). MS (ESI) [M+H]⁺=310.2.

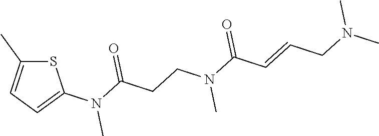

Example 61: (E)-4-(dimethylamino)-N-methyl-N-(3-(methyl(5-methylthiophen-2-yl)amino)-3-oxopropyl)but-2-enamide (XS175-160). Example 61 was synthesized following similar procedure for preparing example 28. White solid, 23% yield. ¹H NMR (400 MHz, Methanol-d₄) δ 6.86-6.70 (m, 2H), 6.64 (s, 1H), 6.40 (d, J=15.4 Hz, 1H), 4.12 (d, J=7.2 Hz, 2H), 3.52-3.43 (m, 2H), 3.23 (s, 3H), 3.15 (s, 9H), 2.45 (s, 3H). MS (ESI) [M+H]⁺=324.2.

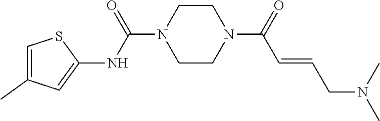

Example 62: (E)-1-(4-(dimethylamino)but-2-enoyl)-N-(4-methylthiophen-2-yl)piperidine-4-carboxamide (XS175-173). Example 62 was synthesized following similar procedure for preparing example 28. White solid, 46% yield. ¹H NMR (400 MHz, Methanol-d₄) δ 6.93 (d, J=15.1 Hz, 1H), 6.62 (dt, J=14.6, 6.8 Hz, 1H), 6.51 (s, 1H), 4.54 (d, J=12.9 Hz, 1H), 4.12 (d, J=13.4 Hz, 1H), 3.90 (d, J=6.8 Hz, 2H), 3.21 (t, J=12.9 Hz, 1H), 2.87 (s, 7H), 2.70-2.58 (m, 1H), 2.13 (s, 3H), 1.89 (d, J=12.5 Hz, 2H), 1.78-1.52 (m, 2H). MS (ESI) [M+H]⁺=336.2.

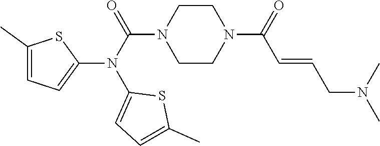

Example 63: (E)-1-(4-(dimethylamino)but-2-enoyl)-N,N-bis(5-methylthiophen-2-yl)piperidine-4-carboxamide (XS175-174). Example 63 was synthesized following similar procedure for preparing example 28. White solid, 13% yield. ¹H NMR (400 MHz, Methanol-d₄) δ 7.17-6.88 (m, 2H), 6.77 (s, 1H), 6.70-6.58 (m, 1H), 6.47 (s, 1H), 6.21 (s, 1H), 4.53 (d, J=13.1 Hz, 1H), 4.09 (d, J=13.5 Hz, 1H), 3.93 (d, J=7.0 Hz, 2H), 3.06 (t, J=12.8 Hz, 1H), 3.01-2.94 (m, 1H), 2.94-2.86 (m, 6H), 2.66 (t, J=12.9 Hz, 1H), 2.45 (d, J=49.1 Hz, 6H), 1.89 (d, J=13.0 Hz, 2H), 1.82-1.59 (m, 2H). MS (ESI) [M+H]⁺=432.2.

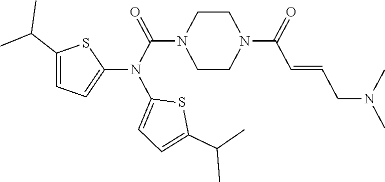 [SMILES: CC(C)c1ccc(N(C(=O)N2CCN(C(=O)/C=C/CN(C)C)CC2)c2ccc(C(C)C)s2)s1]

Example 64: (E)-1-(4-(dimethylamino)but-2-enoyl)-N,N-bis(5-isopropylthiophen-2-yl)piperidine-4-carboxamide (XS175-175). Example 64 was synthesized following similar procedure for preparing example 28. White solid, 19% yield. ¹H NMR (400 MHz, Methanol-d₄) δ 7.18-6.89 (m, 2H), 6.81 (d, J=18.9 Hz, 1H), 6.70-6.60 (m, 1H), 6.60-6.38 (m, 1H), 6.26 (s, 1H), 4.54 (d, J=13.4 Hz, 1H), 4.10 (d, J=13.9 Hz, 1H), 3.98-3.86 (m, 2H), 3.29-3.02 (m, 4H), 2.92 (s, 7H), 2.76-2.57 (m, 1H), 1.91 (d, J=13.3 Hz, 2H), 1.84-1.64 (m, 2H), 1.34 (s, 12H). MS (ESI) [M+H]⁺=488.3.

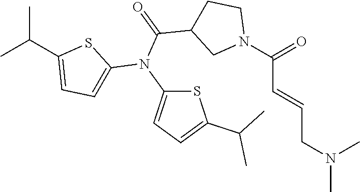 [SMILES: CC(C)c1ccc(N(C(=O)C2CCN(C(=O)/C=C/CN(C)C)C2)c2ccc(C(C)C)s2)s1]

Example 65: (E)-1-(4-(dimethylamino)but-2-enoyl)-N,N-bis(5-isopropylthiophen-2-yl)pyrrolidine-3-carboxamide (XS175-176). Example 65 was synthesized following similar procedure for preparing example 28. White solid, 20% yield. ¹H NMR (400 MHz, Methanol-d₄) δ 7.14-6.94 (m, 1H), 6.83 (s, 1H), 6.73 (s, 2H), 6.64-6.46 (m, 1H), 6.28 (s, 1H), 3.95 (s, 2H), 3.90-3.72 (m, 2H), 3.66 (t, J=7.5 Hz, 1H), 3.57-3.37 (m, 2H), 3.27-3.02 (m, 2H), 2.92 (s, 6H), 2.40-2.09 (m, 2H), 1.38-1.28 (m, 12H). MS (ESI) [M+H]⁺=474.3.

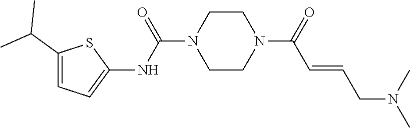 [SMILES: CC(C)c1ccc(NC(=O)N2CCN(C(=O)/C=C/CN(C)C)CC2)s1]

Example 66: (E)-1-(4-(dimethylamino)but-2-enoyl)-N-(5-isopropylthiophen-2-yl)piperidine-4-carboxamide (XS175-178). Example 66 was synthesized following similar procedure for preparing example 28. White solid, 49% yield. ¹H NMR (400 MHz, Methanol-d₄) δ 6.95 (d, J=15.0 Hz, 1H), 6.71-6.57 (m, 1H), 6.51 (d, J=9.7 Hz, 2H), 4.57 (d, J=12.9 Hz, 1H), 4.14 (d, J=13.4 Hz, 1H), 3.93 (d, J=6.8 Hz, 2H), 3.23 (d, J=12.9 Hz, 1H), 3.11-2.99 (m, 1H), 2.90 (s, 7H), 2.74-2.56 (m, 1H), 1.92 (d, J=13.5 Hz, 2H), 1.80-1.57 (m, 2H). MS (ESI) [M+H]⁺=364.7.

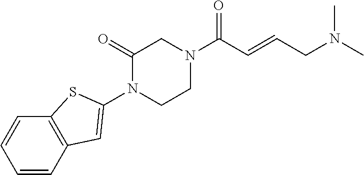 [SMILES: CN(C)C/C=C/C(=O)N1CCN(c2cc3ccccc3s2)C(=O)C1]

Example 67: (E)-1-(benzo[b]thiophen-2-yl)-4-(4-(dimethylamino)but-2-enoyl)piperazin-2-one (XS175-179). Example 67 was synthesized following similar procedure for preparing example 28. White solid, 46% yield. ¹H NMR (400 MHz, Methanol-d₄) δ 7.83-7.60 (m, 2H), 7.40-7.20 (m, 2H), 7.11-6.91 (m, 2H), 6.79 (d, J=14.6 Hz, 1H), 4.54 (d, J=30.3 Hz, 2H), 4.23-3.86 (m, 6H), 2.93 (s, 6H). MS (ESI) [M+H]⁺=344.4.

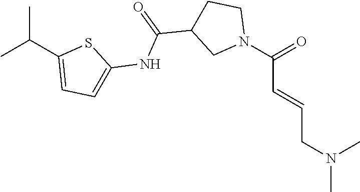 [SMILES: CC(C)c1ccc(NC(=O)C2CCN(C(=O)/C=C/CN(C)C)C2)s1]

Example 68: (E)-1-(4-(dimethylamino)but-2-enoyl)-N-(5-isopropylthiophen-2-yl)pyrrolidine-3-carboxamide (XS175-180). Example 68 was synthesized following similar procedure for preparing example 28. White solid, 37% yield. ¹H NMR (400 MHz, Methanol-d₄) δ 6.76 (t, J=9.3 Hz, 2H), 6.60-6.45 (m, 2H), 3.95 (s, 2H), 3.91-3.49 (m, 4H), 3.28-3.00 (m, 2H), 2.93-2.86 (m, 6H), 2.37-2.08 (m, 2H), 1.28 (d, J=5.4 Hz, 6H). MS (ESI) [M+H]⁺=350.3.

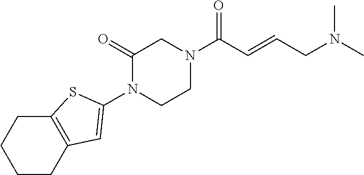 [SMILES: CN(C)C/C=C/C(=O)N1CCN(c2cc3c(s2)CCCC3)C(=O)C1]

Example 69: (E)-4-(4-(dimethylamino)but-2-enoyl)-1-(4,5,6,7-tetrahydrobenzo[b]thiophen-2-yl)piperazin-2-one (XS175-186). Example 69 was synthesized following similar procedure for preparing example 28. White solid, 44% yield. ¹H NMR (400 MHz, Methanol-d₄) δ 7.08-6.84 (m, 1H), 6.77 (q, J=8.0 Hz, 1H), 6.45 (s, 1H), 4.43 (d, J=29.8 Hz, 2H), 4.08-3.78 (m, 6H), 2.90 (s, 6H), 2.64 (s, 2H), 2.52 (s, 2H), 1.87-1.66 (m, 4H). MS (ESI) [M+H]⁺=348.2.

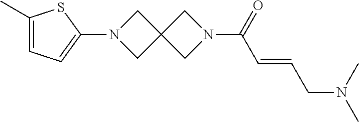

Example 70: (E)-4-(dimethylamino)-1-(6-(5-methylthiophen-2-yl)-2,6-diazaspiro[3.3]heptan-2-yl)but-2-en-1-one (XS186-3). Example 70 was synthesized following similar procedure for preparing example 28. White solid, 23% yield. ¹H NMR (400 MHz, Methanol-d4) δ 6.69 (s, 2H), 6.55-6.32 (m, 2H), 4.50 (s, 2H), 4.22 (d, J=3.2 Hz, 2H), 4.01-3.91 (m, 6H), 2.90-2.88 (m, 6H), 2.32 (d, J=3.4 Hz, 3H). MS (ESI) [M+H]⁺=306.2.

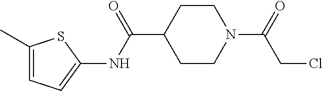 [SMILES: Cc1ccc(NC(=O)C2CCN(C(=O)CCl)CC2)s1]

Example 71: 1-(2-chloroacetyl)-N-(5-methylthiophen-2-yl)piperidine-4-carboxamide (XS185-4). Example 71 was synthesized following similar procedure for preparing example 53. White solid, 29% yield. ¹H NMR (400 MHz, Methanol-d₄) δ 6.50 (s, 2H), 4.52 (d, J=13.3 Hz, 1H), 4.37-4.20 (m, 2H), 4.03 (d, J=13.7 Hz, 1H), 3.25 (t, J=13.2 Hz, 1H), 2.83 (t, J=13.1 Hz, 1H), 2.72-2.57 (m, 1H), 2.42-2.32 (m, 3H), 1.99-1.77 (m, 3H), 1.76-1.59 (m, 1H). MS (ESI) [M+H]⁺=301.1.

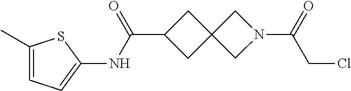 [SMILES: Cc1ccc(NC(=O)C2CC3(C2)CN(C(=O)CCl)C3)s1]

Example 72: 2-(2-chloroacetyl)-N-(5-methylthiophen-2-yl)-2-azaspiro[3.3]heptane-6-carboxamide (XS186-5). Example 72 was synthesized following similar procedure for preparing example 53. White solid, 25% yield. ¹H NMR (400 MHz, Methanol-d₄) δ 6.48 (d, J=9.4 Hz, 2H), 4.32 (d, J=28.4 Hz, 2H), 4.05 (d, J=24.1 Hz, 4H), 3.11 (p, J=7.4 Hz, 1H), 2.50 (d, J=7.6 Hz, 4H), 2.39 (s, 3H). MS (ESI) [M+H]⁺=313.1.

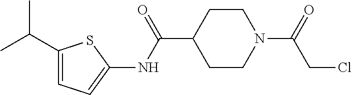 [SMILES: CC(C)c1ccc(NC(=O)C2CCN(C(=O)CCl)CC2)s1]

Example 73: 1-(2-chloroacetyl)-N-(5-isopropylthiophen-2-yl)piperidine-4-carboxamide (XS185-6). Example 73 was synthesized following similar procedure for preparing example 28. White solid, 30% yield. ¹H NMR (400 MHz, Methanol-d₄) δ 6.59-6.45 (m, 2H), 4.60-4.43 (m, 1H), 4.36-4.18 (m, 2H), 4.08-3.95 (m, 1H), 3.25 (t, J=13.1 Hz, 1H), 3.14-3.00 (m, 1H), 2.83 (t, J=12.7 Hz, 1H), 2.71-2.58 (m, 1H), 2.02-1.77 (m, 3H), 1.76-1.62 (m, 1H), 1.34-1.23 (m, 6H). MS (ESI) [M+H]⁺=329.1.

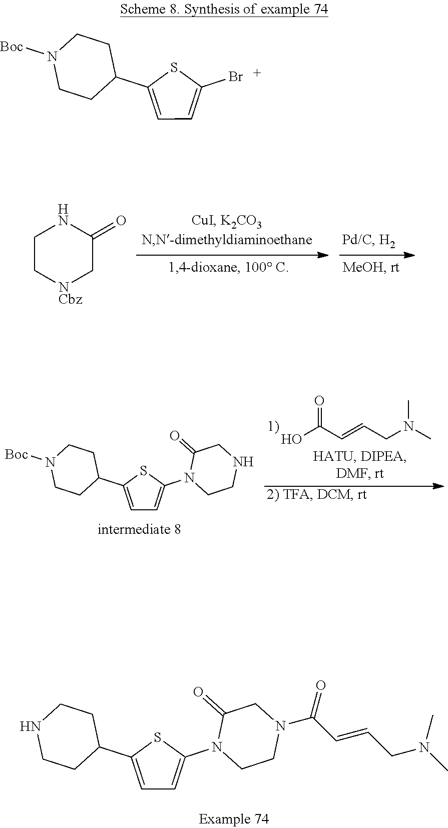

Example 74: (E)-4-(4-(dimethylamino)but-2-enoyl)-1-(5-(piperidin-4-yl)thiophen-2-yl)piperazin-2-one (XS185-24) To a solution of tert-butyl 4-(5-bromothiophen-2-yl)piperidine-1-carboxylate (123.0 mg, 0.35 mmol) dissolved in dioxane (2 mL), N,N′-dimethylethylenediamine (9 μL, 0.105 mmol, 0.3 eq), K₂CO₃(147.2 mg, 1.05 mmol, 3.0 eq), CuI (6.8 mg, 0.035 mmol, 0.1 eq) followed by benzyl 3-oxopiperazine-1-carboxylate (125.1 mg, 0.54 mmol, 1.5 eq). The reaction mixture was stirred at 100° C. under nitrogen atmosphere overnight. After cooling down to rt, resulting crude mixtures were purified via silica gel column then the product dissolved in MeOH (3 mL), and the Pd/C(20 mg) were added. The reaction mixture stirred at hydrogen atmosphere. MeOH was removed then purified via silica gel column chromatography to yield intermediate 8.

To a solution of intermediate 8 was dissovled in DMF (1 mL), HATU (59.3 mg, 0.144 mmol, 1.1 eq) and DIPEA (0.1 mL, 2.0 eq) were added. At rt the (E)-4-(dimethylamino)but-2-enoic acid (17.7 mg, 0.107 mmol, 1.0 eq.) was added, the reaction mixture stirred at rt for 2 h. Resulting crude mixtures were purified via prep-HPLC then re-dissolved in DCM (0.5 mL), and the TFA(0.5 mL) were added. The reaction mixture stirred at rt 1 h. Excess TFA was removed, resulting crude product was purified via prep-HPLC to yield title compound (White solid, 19% yield)¹H NMR (400 MHz, Methanol-d₄) δ 6.95 (dd, J=38.8, 15.4 Hz, 1H), 6.86-6.62 (m, 3H), 4.48 (d, J=29.9 Hz, 2H), 4.16-4.01 (m, 2H), 4.01-3.85 (m, 4H), 3.46 (d, J=13.0 Hz, 2H), 3.12 (t, J=12.5 Hz, 4H), 2.91 (s, 6H), 2.22 (d, J=14.2 Hz, 2H), 1.89 (q, J=13.1 Hz, 2H), 1.45-1.12 (m, 1H). MS (ESI) [M+H]⁺=477.3.

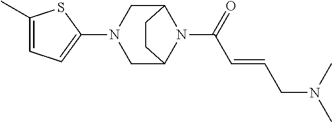

Example 75: (E)-4-(dimethylamino)-1-(3-(5-methylthiophen-2-yl)-3,8-diazabicyclo[3.2.1]octan-8-yl)but-2-en-1-one (XS185-29). Example 75 was synthesized following similar procedure for preparing example 28. White solid, 19% yield. ¹H NMR (400 MHz, Methanol-d₄) δ 6.94-6.74 (m, 3H), 6.36 (s, 1H), 4.78-4.40 (m, 3H), 3.99-3.95 (m, 4H), 2.92 (m, 8H), 2.31 (s, 3H), 2.09-2.03 (m, 2H), 1.98 (td, J=14.5, 12.6, 8.6 Hz, 3H). MS (ESI) [M+H]⁺=320.2.

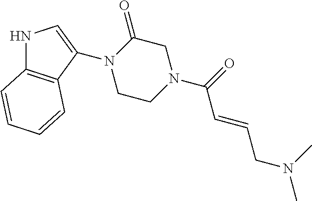 [SMILES: CN(C)C/C=C/C(=O)N1CCN(c2c[nH]c3ccccc23)C(=O)C1]

Example 76: (E)-4-(4-(dimethylamino)but-2-enoyl)-1-(1H-indol-3-yl)piperazin-2-one (XS185-43). Example 76 was synthesized following similar procedure for preparing example 28. White solid, 32% yield. ¹H NMR (400 MHz, Methanol-d4) δ 7.40 (t, J=9.8 Hz, 2H), 7.32 (d, J=3.1 Hz, 1H), 7.14 (d, J=9.3 Hz, 1H), 7.07 (d, J=8.6 Hz, 1H), 7.02-6.87 (m, 1H), 6.81-6.64 (m, 1H), 4.64-4.38 (m, 2H), 4.09 (t, J=5.4 Hz, 2H), 4.02-3.79 (m, 4H), 2.88 (s, 6H). MS (ESI) [M+H]⁺=327.2.

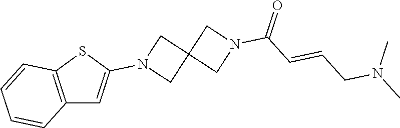 [SMILES: CN(C)C/C=C/C(=O)N1CC2(C1)CN(c1cc3ccccc3s1)C2]

Example 77: (E)-1-(6-(benzo[b]thiophen-2-yl)-2,6-diazaspiro[3.3]heptan-2-yl)-4-(dimethylamino)but-2-en-1-one (XS185-46). Example 77 was synthesized following similar procedure for preparing example 28. White solid, 17% yield. ¹H NMR (400 MHz, Methanol-d₄) δ 7.57 (t, J=6.2 Hz, 1H), 7.43 (t, J=6.1 Hz, 1H), 7.19 (q, J=6.8, 6.2 Hz, 1H), 7.05 (t, J=6.6 Hz, 1H), 6.78-6.39 (m, 3H), 4.53 (d, J=4.7 Hz, 2H), 4.32-4.21 (m, 4H), 4.11 (t, J=3.0 Hz, 3H), 3.94 (t, J=5.7 Hz, 2H), 2.90 (s, 6H). MS (ESI) [M+H]⁺=342.4.

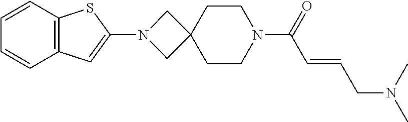 [SMILES: CN(C)C/C=C/C(=O)N1CCC2(CC1)CN(c1cc3ccccc3s1)C2]

Example 78: (E)-1-(2-(benzo[b]thiophen-2-yl)-2,7-diazaspiro[3.5]nonan-7-yl)-4-(dimethylamino)but-2-en-1-one (XS185-59). Example 78 was synthesized following similar procedure for preparing example 28. White solid, 19% yield. ¹H NMR (400 MHz, Methanol-d₄) δ 7.59-7.49 (m, 1H), 7.44-7.34 (m, 1H), 7.17 (d, J=9.1 Hz, 1H), 7.06-6.89 (m, 2H), 6.76-6.49 (m, 2H), 3.94 (t, J=4.2 Hz, 4H), 3.78-3.77 (m, 2H), 3.72-3.62 (m, 4H), 2.91 (s, 7H), 1.94-1.85 (m, 4H). MS (ESI) [M+H]⁺=370.2.

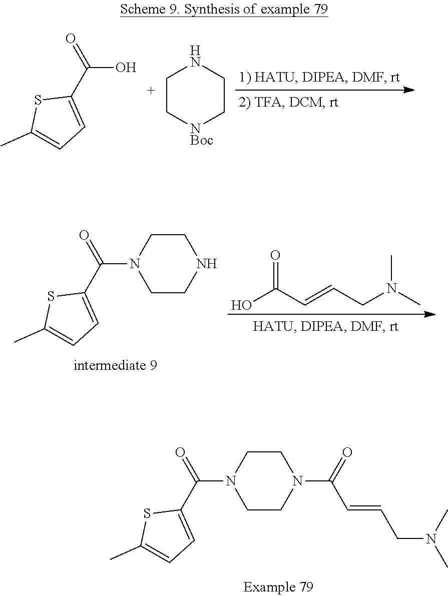

Example 79: (E)-4-(dimethylamino)-1-(4-(5-methylthiophene-2-carbonyl)piperazin-1-yl)but-2-en-1-one (XS185-64). To a solution of 5-methylthiophene-2-carboxylic acid(75 mg, 0.5 mmol, 1.0 eq) was dissovled in DMF (1 mL), HATU (209 mg, 0.55 mmol, 1.1 eq) and DIPEA (0.18 mL, 2.0 eq) were added. At rt the tert-butyl piperazine-1-carboxylate (93.3 mg, 0.5 mmol, 1.0 eq.) was added, the reaction mixture stirred at rt for 2 h. Resulting crude mixtures were purified via prep-HPLC then re-dissolved in DCM (0.5 mL), and the TFA(0.5 mL) were added. The reaction mixture stirred at rt 1 h. Excess TFA was removed, resulting crude product was purified via prep-HPLC to yield intermediate 9.

To a solution of intermediate 9(20 mg, 0.095 mmol) was dissovled in DMF (1 mL), HATU (39.8 mg, 0.1 mmol, 1.1 eq) and DIPEA (0.1 mL, 2.0 eq) were added. At rt the (E)-4-(dimethylamino)but-2-enoic acid (15.8 mg, 0.095 mmol, 1.0 eq.) was added, the reaction mixture stirred at rt for 2 h. Resulting crude mixtures were purified via prep-HPLC to yield title compound (White solid, 30% yield) ¹H NMR (400 MHz, Methanol-d₄) δ 7.28-7.18 (m, 1H), 7.01-6.86 (m, 1H), 6.78 (d, J=4.5 Hz, 1H), 6.74-6.60 (m, 1H), 4.02-3.86 (m, 2H), 3.86-3.75 (m, 4H), 3.5-3.62 (m, 4H), 2.87(s 6H), 2.48 (s, 3H), MS (ESI) [M+H]⁺=322.2.

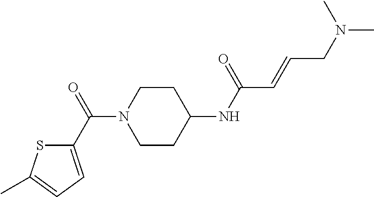

Example 80: (E)-4-(dimethylamino)-N-(1-(5-methylthiophene-2-carbonyl)piperidin-4-yl)but-2-enamide (XS185-65). Example 80 was synthesized following similar procedure for preparing example 79. White solid, 46% yield. ¹H NMR (400 MHz, Methanol-d₄) δ 7.11 (t, J=4.0 Hz, 1H), 6.72 (d, J=4.8 Hz, 1H), 6.69-6.56 (m, 1H), 6.35-6.19 (m, 1H), 4.28 (d, J=13.5 Hz, 2H), 4.06-3.89 (m, 1H), 3.84 (t, J=5.5 Hz, 2H), 3.20-3.03 (m, 2H), 2.82 (s, 6H), 2.42 (s, 3H), 1.90 (d, J=12.9 Hz, 2H), 1.54-1.35 (m, 2H). MS (ESI) [M+H]⁺=336.2.

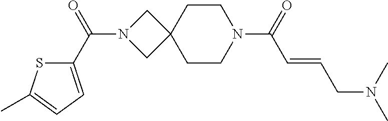

Example 81: (E)-4-(dimethylamino)-1-(2-(5-methylthiophene-2-carbonyl)-2,7-diazaspiro[3.5]nonan-7-yl)but-2-en-1-one (XS185-66). Example 81 was synthesized following similar procedure for preparing example 79. White solid, 31% yield. ¹H NMR (400 MHz, Methanol-d4) δ 7.35-7.27 (m, 1H), 6.96-6.85 (m, 1H), 6.77 (d, J=4.5 Hz, 1H), 6.67-6.49 (m, 1H), 4.20 (s, 2H), 3.95-3.73 (m, 4H), 3.71-3.47 (m, 4H), 2.82 (s, 6H), 2.43 (s, 3H), 1.88-1.61 (m, 4H). MS (ESI) [M+H]⁺=362.2.

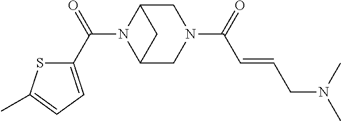

Example 82: (E)-4-(dimethylamino)-1-(6-(5-methylthiophene-2-carbonyl)-3,6-diazabicyclo[3.1.1]heptan-3-yl)but-2-en-1-one (XS185-67). Example 82 was synthesized following similar procedure for preparing example 79. White solid, 57% yield. ¹H NMR (400 MHz, Methanol-d₄) δ 7.48-7.31 (m, 1H), 6.88-6.73 (m, 2H), 6.73-6.60 (m, 1H), 5.12-4.89 (m, 1H), 4.71-4.42 (m, 1H), 4.35-4.00 (m, 1H), 3.96-3.75 (m, 4H), 3.73-3.60 (m, 1H), 2.88-2.76 (m, 7H), 2.46 (s, 3H), 1.69-1.57 (m, 1H). MS (ESI) [M+H]⁺=334.2.

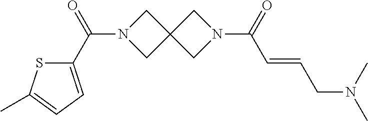

Example 83: (E)-4-(dimethylamino)-1-(6-(5-methylthiophene-2-carbonyl)-2,6-diazaspiro[3.3]heptan-2-yl)but-2-en-1-one (XS185-69). Example 83 was synthesized following similar procedure for preparing example 79. White solid, 19% yield. ¹H NMR (400 MHz, Methanol-d₄) δ 7.27 (d, J=4.1 Hz, 1H), 6.75 (d, J=4.6 Hz, 1H), 6.73-6.54 (m, 1H), 6.49-6.33 (m, 1H), 4.59 (s, 2H), 4.50-4.39 (m, 2H), 4.32-4.12 (m, 4H), 3.85 (t, J=5.4 Hz, 2H), 2.81 (s, 6H), 2.42 (s, 3H). MS (ESI) [M+H]⁺=334.2.

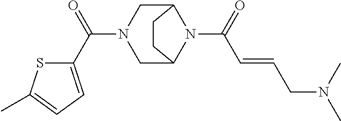

Example 84: (E)-4-(dimethylamino)-1-(3-(5-methylthiophene-2-carbonyl)-3,8-diazabicyclo[3.2.1]octan-8-yl)but-2-en-1-one (XS185-70). Example 84 was synthesized following similar procedure for preparing example 79. White solid, 46% yield. ¹H NMR (400 MHz, Methanol-d₄) δ 7.30-7.17 (m, 1H), 7.01-6.88 (m, 1H), 6.88-6.72 (m, 2H), 4.76 (s, 1H), 4.61 (t, J=4.8 Hz, 1H), 4.38 (d, J=15.7 Hz, 2H), 3.99 (t, J=5.1 Hz, 2H), 3.34 (s, 3H), 2.94 (s, 6H), 2.53 (s, 3H), 2.22-1.70 (m, 4H). MS (ESI) [M+H]⁺=348.2.

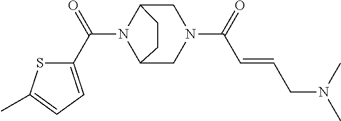

Example 85: (E)-4-(dimethylamino)-1-(8-(5-methylthiophene-2-carbonyl)-3,8-diazabicyclo[3.2.1]octan-3-yl)but-2-en-1-one (XS185-71). Example 85 was synthesized following similar procedure for preparing example 79. White solid, 49% yield. ¹H NMR (400 MHz, Methanol-d₄) δ 7.38-7.29 (m, 1H), 6.94-6.82 (m, 1H), 6.80 (d, J=4.6 Hz, 1H), 6.76-6.57 (m, 1H), 4.74 (s, 2H), 4.48-4.30 (m, 1H), 4.00-3.82 (m, 3H), 3.56-3.41 (m, 1H), 3.09-2.98 (m, 1H), 2.86 (s, 6H), 2.47 (s, 3H), 2.05-1.83 (m, 2H), 1.83-1.59 (m, 2H). MS (ESI) [M+H]⁺=348.2.

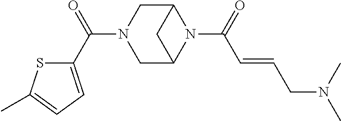

Example 86: (E)-4-(dimethylamino)-1-(3-(5-methylthiophene-2-carbonyl)-3,6-diazabicyclo[3.1.1]heptan-6-yl)but-2-en-1-one (XS185-72). White solid, 54% yield. Example 86 was synthesized following similar procedure for preparing example 79. ¹H NMR (400 MHz, Methanol-d₄) δ 7.35 (d, J=3.8 Hz, 1H), 6.75 (s, 1H), 6.73-6.63 (m, 1H), 6.60-6.44 (m, 1H), 4.73 (s, 1H), 4.49 (s, 1H), 4.43-3.74 (m, 6H), 2.82 (s, 7H), 2.43 (s, 3H), 1.77-1.54 (m, 1H). MS (ESI) [M+H]⁺=334.6.

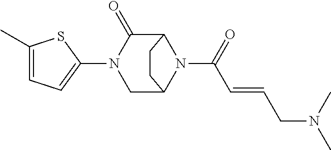

Example 87: (E)-8-(4-(dimethylamino)but-2-enoyl)-3-(5-methylthiophen-2-yl)-3,8-diazabicyclo[3.2.1]octan-2-one (XS185-75). Example 87 was synthesized following similar procedure for preparing example 28. White solid, 39% yield. ¹H NMR (400 MHz, Methanol-d₄) δ 7.05-6.73 (m, 2H), 6.55 (d, J=8.4 Hz, 2H), 5.07 (d, J=37.4 Hz, 2H), 4.21-3.90 (m, 3H), 3.84-3.59 (m, 1H), 2.93 (s, 6H), 2.53-1.88 (m, 7H). MS (ESI) [M+H]⁺=334.2.

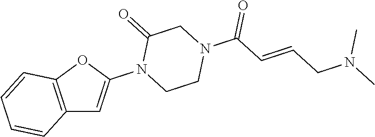 [SMILES: CN(C)C/C=C/C(=O)N1CCN(c2cc3ccccc3o2)C(=O)C1]

Example 88: (E)-1-(benzofuran-2-yl)-4-(4-(dimethylamino)but-2-enoyl)piperazin-2-one (XS185-78) Example 88 was synthesized following similar procedure for preparing example 28. White solid, 36% yield. ¹H NMR (400 MHz, Methanol-d4) δ 7.55 (d, J=5.3 Hz, 1H), 7.44 (s, 1H), 7.32-7.20 (m, 2H), 6.91 (d, J=3.0 Hz, 1H), 6.78 (s, 2H), 4.54 (d, J=28.2 Hz, 2H), 4.14 (d, J=37.0 Hz, 4H), 3.99 (d, J=8.4 Hz, 3H), 3.87-3.73 (m, 1H), 2.93 (s, 6H). MS (ESI) [M+H]⁻=328.2.

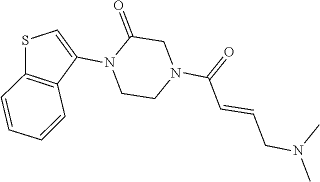 [SMILES: CN(C)C/C=C/C(=O)N1CCN(c2csc3ccccc23)C(=O)C1]

Example 89: (E)-1-(benzo[b]thiophen-3-yl)-4-(4-(dimethylamino)but-2-enoyl)piperazin-2-one (XS185-86). Example 89 was synthesized following similar procedure for preparing example 28. White solid, 40% yield. ¹H NMR (400 MHz, Methanol-d₄) δ 7.93 (dt, J=6.2, 3.8 Hz, 1H), 7.78-7.58 (m, 2H), 7.52-7.37 (m, 2H), 7.02 (t, J=17.5 Hz, 1H), 6.89-6.75 (m, 1H), 4.57 (d, J=26.5 Hz, 2H), 4.16 (t, J=5.3 Hz, 2H), 4.06-3.97 (m, 2H), 3.95-3.80 (m, 2H), 2.94 (s, 6H). MS (ESI) [M+H]⁺=344.1

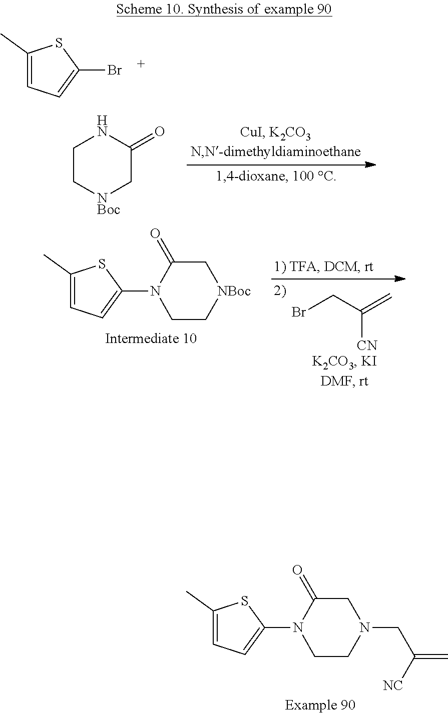

Example 90: 2-((4-(5-methylthiophen-2-yl)-3-oxopiperazin-1-yl)methyl)acrylonitrile (XS185-87). To a solution of 2-bromo-5-methylthiophene (50.0 mg, 0.28 mmol) dissolved in dioxane (1 mL), N,N′-dimethyl ethylenediamine (9 μL, 0.0846 mmol, 0.3 eq), K₂CO₃(116.9 mg, 0.846 mmol, 3.0 eq), CuI (5.4 mg, 0.028 mmol, 0.1 eq) followed by tert-butyl 3-oxopiperazine-1-carboxylate (84.7 mg, 0.42 mmol, 1.5 eq). The reaction mixture was stirred at 100° C. under nitrogen atmosphere overnight. After cooling down to rt, resulting crude mixtures were purified via silica gel column chromatography to yield intermediate 1.

To a solution of intermediate 1 (20 mg, 0.102 mmol) dissolved in DCM (0.5 mL), and the TFA(0.5 mL) were added. The reaction mixture stirred at rt 1 h. Excess TFA was removed, resulting crude product was re-dissolved in DMF (1 mL), K₂CO₃ (28.2 mg, 0.2 mmol, 2.0 eq) and KI (16.9 mg, 1.0 eq) were added. At rt the 2-(bromomethyl)acrylonitrile (17.9 mg, 0.124 mmol, 1.2 eq.) was added, the reaction mixture stirred at rt for 5 h. Resulting crude mixtures were purified via prep-HPLC to yield title compound (White solid, 24% yield)¹H NMR (400 MHz, Methanol-d₄) δ 6.62 (d, J=14.6 Hz, 2H), 6.36-6.13 (m, 2H), 3.92 (d, J=5.2 Hz, 2H), 3.55 (t, J=5.1 Hz, 4H), 3.15 (d, J=5.4 Hz, 2H), 2.41 (s, 3H). MS (ESI) [M+H]⁺=262.1.

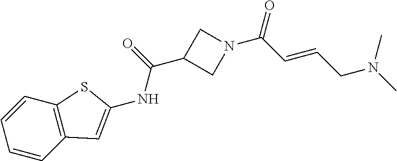 [SMILES: CN(C)C/C=C/C(=O)N1CC(C(=O)Nc2cc3ccccc3s2)C1]

Example 91: (E)-N-(benzo[b]thiophen-2-yl)-1-(4-(dimethylamino)but-2-enoyl)azetidine-3-carboxamide (XS185-90). Example 91 was synthesized following similar procedure for preparing example 53. White solid, 34% yield. ¹H NMR (400 MHz, Methanol-d₄) δ 7.67-7.57 (m, 1H), 7.56-7.44 (m, 1H), 7.25-7.15 (m, 1H), 7.16-7.06 (m, 1H), 6.87 (t, J=2.8 Hz, 1H), 6.73-6.55 (m, 1H), 6.51-6.35 (m, 1H), 4.56-4.34 (m, 2H), 4.30-4.07 (m, 2H), 3.85 (t, J=5.3 Hz, 2H), 3.65-3.51 (m, 1H), 2.79 (s, 6H). MS (ESI) [M+H]⁺=344.1.

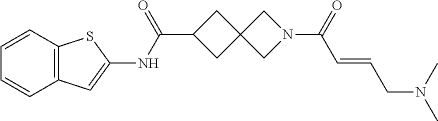 [SMILES: CN(C)C/C=C/C(=O)N1CC2(CC(C(=O)Nc3cc4ccccc4s3)C2)C1]

Example 92: (E)-N-(benzo[b]thiophen-2-yl)-2-(4-(dimethylamino)but-2-enoyl)-2-azaspiro[3.3]heptane-6-carboxamide (XS185-91). Example 92 was synthesized following similar procedure for preparing example 53. White solid, 48% yield. ¹H NMR (400 MHz, Methanol-d₄) δ 7.74-7.64 (m, 1H), 7.63-7.50 (m, 1H), 7.24 (d, J=8.4 Hz, 1H), 7.18 (d, J=8.5 Hz, 1H), 6.88 (d, J=3.6 Hz, 1H), 6.74-6.59 (m, 1H), 6.51-6.36 (m, 1H), 4.41-4.26 (m, 2H), 4.14-4.01 (m, 2H), 3.99-3.85 (m, 2H), 3.24-3.09 (m, 1H), 2.86 (s, 6H), 2.51 (d, J=5.0 Hz, 4H). MS (ESI) [M+H]⁺=384.2.

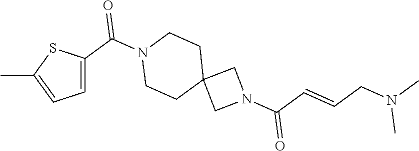

Example 93: (E)-4-(dimethylamino)-1-(7-(5-methylthiophene-2-carbonyl)-2,7-diazaspiro[3.5]nonan-2-yl)but-2-en-1-one (XS185-96). Example 93 was synthesized following similar procedure for preparing example 79. White solid, 16% yield. ¹H NMR (400 MHz, Methanol-d₄) δ 7.15 (d, J=3.7 Hz, 1H), 6.78-6.73 (m, 1H), 6.73-6.63 (m, 1H), 6.46 (d, J=15.3 Hz, 1H), 4.06 (s, 2H), 3.96-3.87 (m, 2H), 3.81 (s, 2H), 3.74-3.58 (m, 4H), 2.85 (s, 6H), 2.50-2.42 (m, 3H), 1.89-1.71 (m, 4H). MS (ESI) [M+H]⁺=362.2.

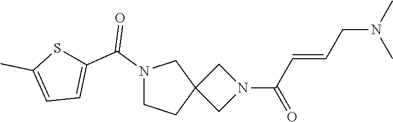

Example 94: (E)-4-(dimethylamino)-1-(6-(5-methylthiophene-2-carbonyl)-2,6-diazaspiro[3.4]octan-2-yl)but-2-en-1-one (XS185-97). Example 94 was synthesized following similar procedure for preparing example 79. White solid, 12% yield. ¹H NMR (400 MHz, Methanol-d₄) δ 7.40 (d, J=3.8 Hz, 1H), 6.85-6.76 (m, 1H), 6.76-6.59 (m, 1H), 6.56-6.31 (m, 1H), 4.39-4.15 (m, 2H), 4.13-3.95 (m, 3H), 3.88 (t, J=10.4 Hz, 3H), 3.69 (d, J=53.8 Hz, 2H), 2.85 (s, 6H), 2.51-2.43 (m, 3H), 2.32-2.08 (m, 2H). MS (ESI) [M+H]⁺=348.2.

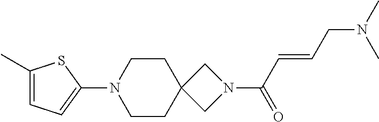

Example 95: (E)-4-(dimethylamino)-1-(7-(5-methylthiophen-2-yl)-2,7-diazaspiro[3.5]nonan-2-yl)but-2-en-1-one (XS185-101). Example 95 was synthesized following similar procedure for preparing example 28. White solid, 18% yield. ¹H NMR (400 MHz, Methanol-d₄) δ 6.82-6.68 (m, 1H), 6.64-6.58 (m, 1H), 6.58-6.47 (m, 2H), 4.84 (s, 2H), 4.60-4.50 (m, 1H), 4.41-4.27 (m, 2H), 4.09 (d, J=10.5 Hz, 1H), 4.01-3.94 (m, 2H), 3.93-3.84 (m, 2H), 2.93 (s, 6H), 2.64-2.57 (m, 2H), 2.43 (s, 3H). MS (ESI) [M+H]⁺=334.2.

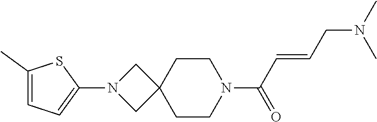

Example 96: (E)-4-(dimethylamino)-1-(2-(5-methylthiophen-2-yl)-2,7-diazaspiro[3.5]nonan-7-yl)but-2-en-1-one (XS185-109). Example 96 was synthesized following similar procedure for preparing example 28. White solid, 20% yield. ¹H NMR (400 MHz, Methanol-d₄) δ 7.64 (s, 1H), 6.95 (d, J=15.1 Hz, 1H), 6.71-6.55 (m, 1H), 4.53 (d, J=2.3 Hz, 1H), 4.33 (s, 2H), 3.92 (d, J=7.3 Hz, 3H), 3.72-3.53 (m, 4H), 2.89 (s, 6H), 2.08-1.90 (m, 4H). MS (ESI) [M+H]⁺=334.2.

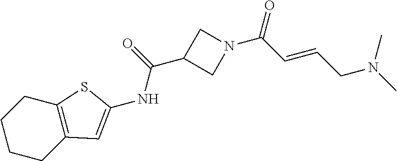 [SMILES: CN(C)C/C=C/C(=O)N1CC(C(=O)Nc2cc3c(s2)CCCC3)C1]

Example 97: (E)-4-(dimethylamino)-1-(2-(5-methylthiophen-2-yl)-2,7-diazaspiro[3.5]nonan-7-yl)but-2-en-1-one (XS185-113). Example 97 was synthesized following similar procedure for preparing example 53. White solid, 43% yield. ¹H NMR (400 MHz, Methanol-d₄) δ 6.76-6.61 (m, 1H), 6.50-6.39 (m, 1H), 6.34 (s, 1H), 4.51-4.37 (m, 2H), 4.22 (t, J=9.6 Hz, 1H), 4.18-4.08 (m, 1H), 3.96-3.83 (m, 2H), 3.61-3.49 (m, 1H), 2.86 (s, 6H), 2.60 (t, J=5.9 Hz, 2H), 2.53-2.40 ((m, 2H), 1.85-1.63 (m, 4H). MS (ESI) [M+H]⁺=348.2.

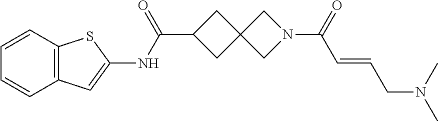 [SMILES: CN(C)C/C=C/C(=O)N1CC2(CC(C(=O)Nc3cc4ccccc4s3)C2)C1]

Example 98: (E)-2-(4-(dimethylamino)but-2-enoyl)-N-(4,5,6,7-tetrahydrobenzo[b]thiophen-2-yl)-2-azaspiro[3.3]heptane-6-carboxamide (XS185-114). Example 98 was synthesized following similar procedure for preparing example 53. White solid, 49% yield. ¹H NMR (400 MHz, Methanol-d₄) δ 6.68-6.51 (m, 1H), 6.40-6.30 (m, 1H), 6.23 (s, 1H), 4.22 (d, J=26.3 Hz, 2H), 3.96 (d, 0.1=22.1 Hz, 2H), 3.89-3.79 (m, 2H), 3.12-2.94 (m, 1H), 2.79 (s, 6H), 2.54 (d, J=5.7 Hz, 2H), 2.46-2.25 (m, 6H), 1.81-1.58 (m, 4H). MS (ESI) [M+H]⁺=388.2.

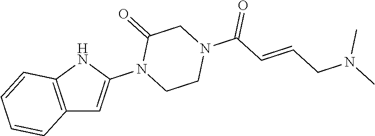 [SMILES: CN(C)C/C=C/C(=O)N1CCN(c2cc3ccccc3[nH]2)C(=O)C1]

Example 99: (E)-4-(4-(dimethylamino)but-2-enoyl)-1-(1H-indol-2-yl)piperazin-2-one (XS185-116). Example 99 was synthesized following similar procedure for preparing example 28. White solid, 37% yield. ¹H NMR (400 MHz, Methanol-d₄) δ 7.47-7.36 (m, 1H), 7.32 (d, J=8.0 Hz, 1H), 7.22-7.00 (m, 2H), 7.00-6.91 (m, 1H), 6.91-6.62 (m, 2H), 4.54-4.14 (m, 2H), 4.03 (q, J=7.1, 6.2 Hz, 1H), 3.99-3.83 (m, 3H), 3.83-3.65 (m, 2H), 2.88-2.78 (m, 6H). MS (ESI) [M+H]⁺=327.2.

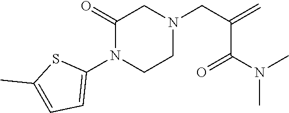

Example 100: N,N-dimethyl-2-((4-(5-methylthiophen-2-yl)-3-oxopiperazin-1-yl)methyl)acrylamide (XS185-118). Example 100 was synthesized following similar procedure for preparing example 90. White solid, 23% yield. ¹H NMR (400 MHz, Methanol-d₄) δ 6.64 (d, J=3.9 Hz, 1H), 6.60-6.53 (m, 1H), 5.94 (s, 1H), 5.77 (s, 1H), 4.14-3.89 (m, 6H), 3.64 (t, J=5.7 Hz, 2H), 3.12 (s, 3H), 2.98 (s, 3H), 2.37 (s, 3H). MS (ESI) [M+H]⁺=308.2.

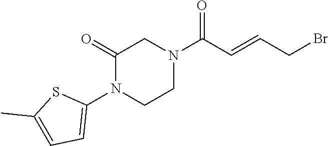 [SMILES: Cc1ccc(N2CCN(C(=O)/C=C/CBr)CC2=O)s1]

Example 101: (E)-4-(4-bromobut-2-enoyl)-1-(5-methylthiophen-2-yl)piperazin-2-one (XS185-122). Example 101 was synthesized following similar procedure for preparing example 28. White solid, 28% yield. ¹H NMR (400 MHz, Methanol-d₄) δ 6.64 (d, J=3.9 Hz, 1H), 6.63-6.57 (m, 1H), 6.56-6.48 (m, 1H), 6.48-6.38 (m, 1H), 4.41 (d, J=26.2 Hz, 2H), 4.03-3.92 (m, 4H), 3.88 (t, J=5.5 Hz, 1H), 3.52-3.38 (m, 2H), 2.41 (s, 3H). MS (ESI) [M+H]⁺=345.0.

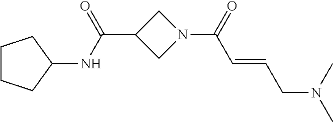 [SMILES: CN(C)C/C=C/C(=O)N1CC(C(=O)NC2CCCC2)C1]

Example 102: (E)-N-cyclopentyl-1-(4-(dimethylamino)but-2-enoyl)azetidine-3-carboxamide (XS185-131). Example 102 was synthesized following similar procedure for preparing example 53. White solid, 21% yield. ¹H NMR (400 MHz, Methanol-d₄) δ 6.78-6.65 (m, 1H), 6.52-6.43 (m, 1H), 4.50-4.31 (m, 2H), 4.25-4.04 (m, 3H), 3.98-3.87 (m, 2H), 3.49-3.34 (m, 1H), 2.89 (s, 6H), 2.02-1.84 (m, 2H), 1.81-1.53 (m, 4H), 1.52-1.39 (m, 2H). MS (ESI) [M+H]⁺=280.2.

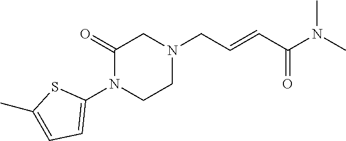

Example 103: (E)-N,N-dimethyl-4-(4-(5-methylthiophen-2-yl)-3-oxopiperazin-1-yl)but-2-enamide (XS185-134). Example 103 was synthesized following similar procedure for preparing example 90. White solid, 19% yield. ¹H NMR (400 MHz, Methanol-d₄) δ 6.98 (d, J=15.0 Hz, 1H), 6.74-6.59 (m, 3H), 4.16-4.02 (m, 6H), 3.72 (t, J=5.7 Hz, 2H), 3.17 (s, 3H), 3.03 (s, 3H), 2.41 (s, 3H). MS (ESI) [M+H]⁺=308.2.

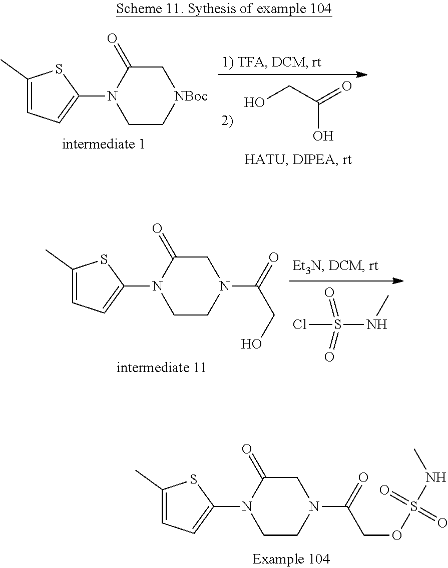

Example 104: (2-(4-(5-methylthiophen-2-yl)-3-oxopiperazin-1-yl)-2-oxoethyl methylsulfamate (XS185-135). To a solution of intermediate 1 (20 mg, 0.102 mmol) dissolved in DMF (1 mL), and the HATU(42.7 mg, 0.112 mmol, 1.1 eq), DIPEA(0.1 mL), 2-hydroxyacetic acid(9.4 mg, 0.102 mmol, 1.0 eq) were added. The reaction mixture stirred at rt 1 h, the crude mixtures were purified by HPLC yield the intermediate 10.

Then the intermediate 10 was re-dissovled in DCM (1 mL), Et₃N (0.1 mL, 2.0 eq) and methylsulfamoyl chloride (9.3 mg, 1.2 eq) were added. The reaction mixture stirred at rt for 5 h. Resulting crude mixtures were purified by HPLC yield the title example 104. White solid, 29% yield. ¹H NMR (400 MHz, Methanol-d4) δ 6.75-6.51 (m, 2H), 4.91 (s, 2H), 4.38 (s, 2H), 3.95 (m, 4H), 2.75 (s, 3H), 2.41 (s, 3H). MS (ESI) [M+H]⁺=348.1.

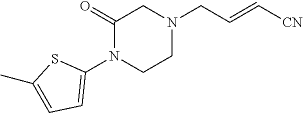 [SMILES: Cc1ccc(N2CCN(C/C=C/C#N)CC2=O)s1]

Example 105: (E)-4-(4-(5-methylthiophen-2-yl)-3-oxopiperazin-1-yl)but-2-enenitrile (XS185-138). Example 105 was synthesized following similar procedure for preparing example 90. White solid, 30% yield. ¹H NMR (400 MHz, Methanol-d₄) δ 6.84-6.61 (m, 1H), 6.57 (d, J=3.9 Hz, 1H), 6.55-6.48 (m, 1H), 5.99-5.77 (m, 1H), 3.93-3.86 (m, 2H), 3.80-3.72 (m, 1H), 3.66 (d, J=6.5 Hz, 2H), 3.64-3.59 (m, 1H), 3.34-3.24 (m, 2H), 2.34 (s, 3H). MS (ESI) [M+H]⁻=262.1.

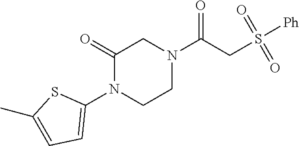 [SMILES: Cc1ccc(N2CCN(C(=O)CS(=O)(=O)c3ccccc3)CC2=O)s1]

Example 106: 1-(5-methylthiophen-2-yl)-4-(2-(phenylsulfonyl)acetyl)piperazin-2-one (XS185-140). Example 106 was synthesized following similar procedure for preparing example 104. White solid, 35% yield. ¹H NMR (400 MHz, Methanol-d₄) δ 8.03-7.90 (m, 2H), 7.82-7.53 (m, 3H), 6.68-6.51 (m, 2H), 4.58 (d, J=18.3 Hz, 2H), 4.36 (d, J=22.2 Hz, 2H), 4.06 (t, J=5.4 Hz, 1H), 4.00-3.89 (m, 2H), 3.78 (t, J=5.5 Hz, 1H), 2.42 (s, 3H). MS (ESI) [M+H]⁺=380.1.

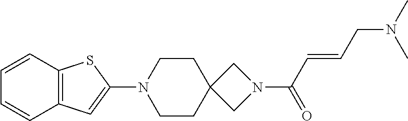 [SMILES: CN(C)C/C=C/C(=O)N1CC2(CCN(c3cc4ccccc4s3)CC2)C1]

Example 107: (E)-1-(7-(benzo[b]thiophen-2-yl)-2,7-diazaspiro[3.5]nonan-2-yl)-4-(dimethylamino)but-2-en-1-one (XS185-147). Example 107 was synthesized following similar procedure for preparing example 28. White solid, 41% yield. ¹H NMR (400 MHz, Methanol-d₄) δ 7.54 (d, J=7.9 Hz, 1H), 7.41 (d, J=7.9 Hz, 1H), 7.16 (t, J=7.6 Hz, 1H), 7.07-6.95 (m, 1H), 6.79-6.63 (m, 1H), 6.55-6.40 (m, 1H), 4.06 (s, 2H), 3.94-3.89 (m, 2H), 3.81 (s, 2H), 3.26-3.16 (m, 4H), 2.88 (s, 7H), 1.93 (t, J=5.6 Hz, 4H). MS (ESI) [M+H]⁺=370.2.

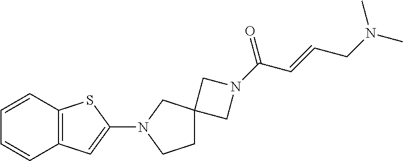 [SMILES: CN(C)C/C=C/C(=O)N1CC2(CCN(c3cc4ccccc4s3)C2)C1]

Example 108: (E)-1-(6-(benzo[b]thiophen-2-yl)-2,6-diazaspiro[3.4]octan-2-yl)-4-(dimethylamino)but-2-en-1-one (XS185-149). Example 108 was synthesized following similar procedure for preparing example 28. White solid, 30% yield. ¹H NMR (400 MHz, Methanol-d₄) δ 7.51 (d, J=7.9 Hz, 1H), 7.35 (d, J=7.9 Hz, 1H), 7.13 (t, J=7.6 Hz, 1H), 6.93 (t, J=7.6 Hz, 1H), 6.79-6.37 (m, 3H), 4.35-4.21 (m, 2H), 4.15-3.96 (m, 2H), 3.96-3.85 (m, 2H), 3.58-3.47 (m, 2H), 3.40 (t, J=7.0 Hz, 2H), 2.86 (s, 7H), 2.27 (t, J=6.8 Hz, 2H).MS (ESI) [M+H]⁻=356.3.

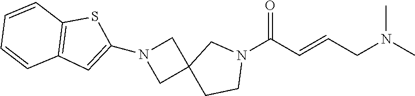 [SMILES: CN(C)C/C=C/C(=O)N1CCC2(C1)CN(c1cc3ccccc3s1)C2]

Example 109: (E)-1-(2-(benzo[b]thiophen-2-yl)-2,6-diazaspiro[3.4]octan-6-yl)-4-(dimethylamino)but-2-en-1-one (XS185-150). Example 109 was synthesized following similar procedure for preparing example 28. White solid, 30% yield. ¹H NMR (400 MHz, Methanol-d₄) δ 7.56 (d, J=8.0 Hz, 1H), 7.42 (d, J=7.9 Hz, 1H), 7.23-7.14 (m, 1H), 7.03 (t, J=7.6 Hz, 1H), 6.83-6.64 (m, 2H), 4.24-4.01 (m, 2H), 3.99-3.86 (m, 5H), 3.79-3.68 (m, 2H), 3.65-3.53 (m, 1H), 2.95-2.84 (m, 6H), 2.38-2.15 (m, 2H).MS (ESI) [M+H]⁺=356.3.

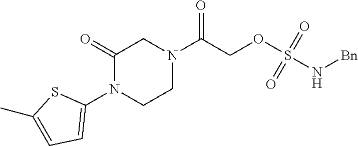 [SMILES: Cc1ccc(N2CCN(C(=O)COS(=O)(=O)NCc3ccccc3)CC2=O)s1]

Example 110: (E)-N-(1-(4-(dimethylamino)but-2-enoyl)azetidin-3-yl)-5-methylthiophene-2-carboxamide (XS185-171). Example 110 was synthesized following similar procedure for preparing example 104. White solid, 15% yield ¹H NMR (400 MHz, DMSO-d₆) δ 8.54-8.35 (m, 1H), 7.34 (d, J=4.1 Hz, 4H), 7.29-7.20 (m, 1H), 6.67-6.49 (m, 2H), 4.81 (d, J=21.1 Hz, 2H), 4.25 (s, 2H), 4.19 (d, J=4.8 Hz, 2H), 3.91-3.82 (m, 2H), 3.82-3.69 (m, 2H), 2.36 (s, 3H).MS (ESI) [M+H]⁺=424.2.

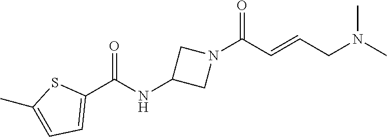

Example 111: (E)-N-(1-(4-(dimethylamino)but-2-enoyl)azetidin-3-yl)-5-methylthiophene-2-carboxamide (XS190-9). Example 111 was synthesized following similar procedure for preparing example 79. White solid, 56% yield ¹H NMR (400 MHz, Methanol-d₄) δ 7.47 (d, J=3.7 Hz, 1H), 6.74 (d, J=3.0 Hz, 1H), 6.71-6.57 (m, 1H), 6.44 (d, J=15.3 Hz, 1H), 4.76-4.63 (m, 1H), 4.58 (t, J=8.6 Hz, 1H), 4.36-4.28 (m, 1H), 4.27-4.18 (m, 1H), 4.07-3.96 (m, 1H), 3.87 (d, J=7.1 Hz, 2H), 2.83 (s, 6H), 2.43 (s, 3H).MS (ESI) [M+H]⁺=307.4.

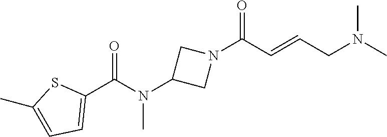

Example 112: (E)-N-(1-(4-(dimethylamino)but-2-enoyl)azetidin-3-yl)-N,5-dimethylthiophene-2-carboxamide (XS190-27). Example 112 was synthesized following similar procedure for preparing example 79. White solid, 23% yield ¹H NMR (400 MHz, Methanol-d₄) δ 7.54 (d, J=3.7 Hz, 1H), 6.89-6.77 (m, 2H), 6.57 (d, J=15.2 Hz, 1H), 4.81-4.70 (m, 1H), 4.67 (t, J=8.5 Hz, 1H), 4.45-4.35 (m, 1H), 4.34-4.24 (m, 1H), 4.14 (d, J=7.2 Hz, 3H), 3.16 (s, 9H), 2.51 (s, 3H).MS (ESI) [M+H]⁺=322.2.

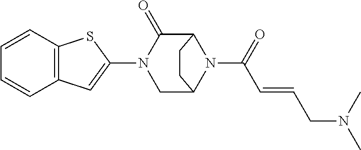 [SMILES: CN(C)C/C=C/C(=O)N1C2CCC1C(=O)N(c1cc3ccccc3s1)C2]

Example 113: (E)-3-(benzo[b]thiophen-2-yl)-8-(4-(dimethylamino)but-2-enoyl)-3,8-diazabicyclo[3.2.1]octan-2-one (XS190-38). Example 113 was synthesized following similar procedure for preparing example 28. White solid, 25% yield ¹H NMR (400 MHz, Methanol-d₄) δ 7.72 (d, J=7.8 Hz, 1H), 7.65 (d, J=7.6 Hz, 1H), 7.34-7.19 (m, 2H), 7.03-6.69 (m, 3H), 5.07 (t, J=35.5 Hz, 2H), 4.12 (t, J=11.3 Hz, 1H), 3.96 (s, 2H), 3.89-3.71 (m, 1H), 2.89 (s, 6H), 2.51-2.16 (m, 3H), 2.16-1.95 (m, 1H).MS (ESI) [M+H]⁺=370.2.

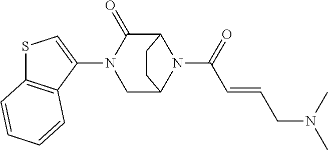 [SMILES: CN(C)C/C=C/C(=O)N1C2CCC1C(=O)N(c1csc3ccccc13)C2]

Example 114: (E)-3-(benzo[b]thiophen-3-yl)-8-(4-(dimethylamino)but-2-enoyl)-3,8-diazabicyclo[3.2.1]octan-2-one (XS190-44). Example 114 was synthesized following similar procedure for preparing example 28. ¹H NMR (400 MHz, Methanol-d₄) δ 7.84 (d, J=4.2 Hz, 1H), 7.58 (s, 1H), 7.41-7.28 (m, 3H), 6.97-6.75 (m, 2H), 5.05-4.88 (m, 2H), 4.08-3.80 (m, 3H), 3.57-3.39 (m, 1H), 2.83 (s, 6H), 2.52-2.11 (m, 4H). White solid, 15% yield MS (ESI) [M+H]⁺=370.2.

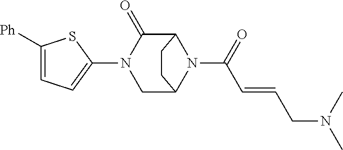 [SMILES: CN(C)C/C=C/C(=O)N1C2CCC1C(=O)N(c1ccc(-c3ccccc3)s1)C2]

Example 115: (E)-8-(4-(dimethylamino)but-2-enoyl)-3-(5-phenylthiophen-2-yl)-3,8-diazabicyclo[3.2.1]octan-2-one (XS190-45). Example 115 was synthesized following similar procedure for preparing example 28. White solid, 18% yield ¹H NMR (400 MHz, Methanol-d₄) δ 7.49-7.41 (m, 2H), 7.23 (t, J=7.7 Hz, 2H), 7.12 (t, J=7.4 Hz, 1H), 7.05 (d, J=4.1 Hz, 1H), 6.91-6.64 (m, 2H), 6.63-6.55 (m, 1H), 5.10-4.89 (m, 2H), 4.05-3.89 (m, 1H), 3.89-3.80 (m, 2H), 3.64 (dd, J=25.2, 11.4 Hz, 1H), 2.80 (s, 3H), 2.78 (s, 3H), 2.38-2.02 (m, 3H), 2.04-1.81 (m, 1H).MS (ESI) [M+H]⁺=396.3.

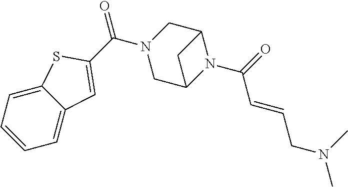 [SMILES: CN(C)C/C=C/C(=O)N1C2CC1CN(C(=O)c1cc3ccccc3s1)C2]

Example 116: (E)-1-(3-(benzo[b]thiophene-2-carbonyl)-3,6-diazabicyclo[3.1.1]heptan-6-yl)-4-(dimethylamino)but-2-en-1-one (XS190-46). Example 116 was synthesized following similar procedure for preparing example 79. White solid, 40% yield ¹H NMR (400 MHz, Methanol-d₄) δ 7.79 (d, J=7.4 Hz, 2H), 7.72 (s, 1H), 7.32 (p, J=7.0 Hz, 2H), 6.74-6.57 (m, 1H), 6.56-6.36 (m, 1H), 4.69 (d, J=17.5 Hz, 1H), 4.47 (d, J=23.4 Hz, 1H), 4.13 (t, J=8.2 Hz, 1H), 4.07-3.96 (m, 1H), 3.94-3.74 (m, 4H), 2.80 (s, 3H), 2.78-2.67 (m, 4H), 1.65 (d, J=9.2 Hz, 1H). MS (ESI) [M+H]⁺=370.2.

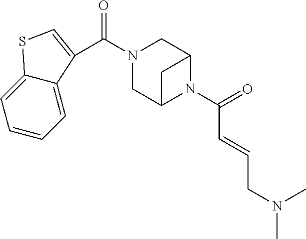 [SMILES: CN(C)C/C=C/C(=O)N1C2CC1CN(C(=O)c1csc3ccccc13)C2]

Example 117: (E)-1-(3-(benzo[b]thiophene-3-carbonyl)-3,6-diazabicyclo[3.1.1]heptan-6-yl)-4-(dimethylamino)but-2-en-1-one (XS190-47). Example 117 was synthesized following similar procedure for preparing example 79. White solid, 50% yield ¹H NMR (400 MHz, Methanol-d₄) δ 7.91-7.81 (m, 1H), 7.75 (d, J=6.4 Hz, 1H), 7.64 (d, J=5.6 Hz, 1H), 7.38-7.26 (m, 2H), 6.75-6.57 (m, 1H), 6.59-6.28 (m, 1H), 4.73 (s, 1H), 4.50 (s, 1H), 4.31-4.06 (m, 1H), 4.01-3.81 (m, 3H), 3.78-3.61 (m, 2H), 3.54 (d, J=11.8 Hz, 1H), 2.81 (s, 3H), 2.76-2.61 (m, 4H), 1.65 (d, J=9.2 Hz, 1H).MS (ESI) [M+H]⁺=370.2.

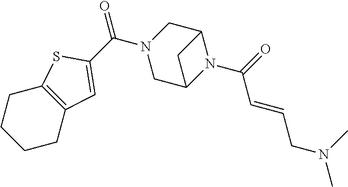 [SMILES: CN(C)C/C=C/C(=O)N1C2CC1CN(C(=O)c1cc3c(s1)CCCC3)C2]

Example 118: (E)-4-(dimethylamino)-1-(3-(4,5,6,7-tetrahydrobenzo[b]thiophene-2-carbonyl)-3,6-diazabicyclo[3.1.1]heptan-6-yl)but-2-en-1-one (XS190-48). Example 118 was synthesized following similar procedure for preparing example 79. White solid, 46% yield ¹H NMR (400 MHz, Methanol-d4) δ 7.15 (s, 1H), 6.70-6.56 (m, 1H), 6.46 (d, J=15.2 Hz, 1H), 4.66 (s, 1H), 4.42 (s, 1H), 4.31-3.69 (m, 6H), 2.83-2.67 (m, 7H), 2.67-2.60 (m, 2H), 2.54-2.42 (m, 2H), 1.70 (dt, J=10.4, 5.4 Hz, 4H), 1.58 (d, J=9.2 Hz, 1H).MS (ESI) [M+H]⁺=374.2.

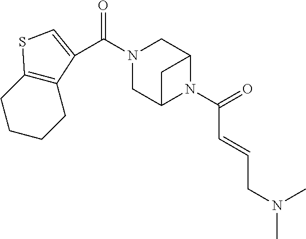 [SMILES: CN(C)C/C=C/C(=O)N1C2CC1CN(C(=O)c1csc3c1CCCC3)C2]

Example 119: (E)-4-(dimethylamino)-1-(3-(4,5,6,7-tetrahydrobenzo[b]thiophene-3-carbonyl)-3,6-diazabicyclo[3.1.1]heptan-6-yl)but-2-en-1-one (XS190-49). Example 119 was synthesized following similar procedure for preparing example 79. White solid, 39% yield ¹H NMR (400 MHz, Methanol-d₄) δ 7.15 (d, J=4.1 Hz, 1H), 6.69-6.54 (m, 1H), 6.52-6.30 (m, 1H), 4.59 (d, J=51.2 Hz, 1H), 4.36 (d, J=73.5 Hz, 1H), 3.88-3.73 (m, 4H), 3.67-3.52 (m, 1H), 3.45 (d, J=12.0 Hz, 1H), 2.79-2.70 (m, 6H), 2.70-2.58 (m, 3H), 2.39-2.28 (m, 2H), 1.77-1.59 (m, 4H), 1.59-1.49 (m, 1H).MS (ESI) [M+H]⁺=374.2.

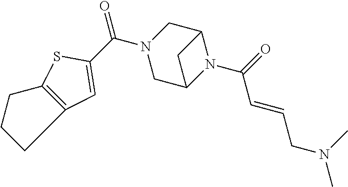 [SMILES: CN(C)C/C=C/C(=O)N1C2CC1CN(C(=O)c1cc3c(s1)CCC3)C2]

Example 120: (E)-1-(3-(5,6-dihydro-4H-cyclopenta[b]thiophene-2-carbonyl)-3,6-diazabicyclo[3.1.1]heptan-6-yl)-4-(dimethylamino)but-2-en-1-one (XS190-50). Example 120 was synthesized following similar procedure for preparing example 79. White solid, 55% yield ¹H NMR (400 MHz, Methanol-d₄) δ 7.26 (s, 1H), 6.72-6.56 (m, 1H), 6.49 (d, J=15.1 Hz, 1H), 4.70 (s, 1H), 4.46 (s, 1H), 4.36-3.72 (m, 6H), 2.86-2.69 (m, 9H), 2.69-2.57 (m, 2H), 2.41-2.24 (m, 2H), 1.62 (d, J=9.2 Hz, 1H).MS ESI [M+H]⁺=360.3.

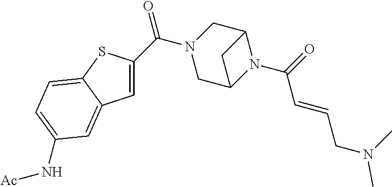 [SMILES: CC(=O)Nc1ccc2sc(C(=O)N3CC4CC(C3)N4C(=O)/C=C/CN(C)C)cc2c1]

Example 121: (E)-N-(2-(6-(4-(dimethylamino)but-2-enoyl)-3,6-diazabicyclo[3.1.1]heptane-3-carbonyl)benzo[b]thiophen-5-yl)acetamide (XS190-59). Example 121 was synthesized following similar procedure for preparing example 79. White solid, 26% yield ¹H NMR (400 MHz, Methanol-d₄)¹H NMR (400 MHz, Methanol-d₄) δ 8.22 (s, 1H), 7.74 (d, 0.1=8.7 Hz, 1H), 7.69 (s, 1H), 7.39 (t, J=9.9 Hz, 1H), 6.75-6.62 (m, 1H), 6.58-6.41 (m, 1H), 4.72 (d, J=17.8 Hz, 1H), 4.50 (d, J=23.8 Hz, 1H), 4.38-3.75 (m, 6H), 2.83 (s, 3H), 2.80-2.71 (m, 4H), 2.08 (s, 3H), 1.68 (d, J=9.2 Hz, 1H).MS (ESI) [M+H]⁺=427.3.

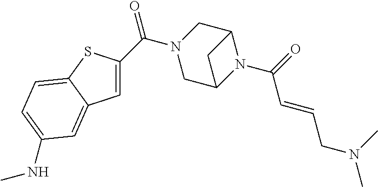

Example 122: (E)-4-(dimethylamino)-1-(3-(5-(methylamino)benzo[b]thiophene-2-carbonyl)-3,6-diazabicyclo[3.1.1]heptan-6-yl)but-2-en-1-one (XS190-68). Example 122 was synthesized following similar procedure for preparing example 79. White solid, 33% yield ¹H NMR (400 MHz, Methanol-d₄) δ 8.06-7.98 (m, 1H), 7.91 (d, J=7.4 Hz, 1H), 7.83 (d, J=6.2 Hz, 1H), 7.42 (d, J=8.6 Hz, 1H), 6.76-6.64 (m, 1H), 6.58-6.44 (m, 1H), 4.73 (d, J=18.3 Hz, 1H), 4.50 (d, J=27.1 Hz, 1H), 4.41-3.81 (m, 6H), 3.07-3.00 (m, 3H), 2.84-2.74 (m, 7H), 1.69 (d, J=9.2 Hz, 1H).MS (ESI) [M+H]⁺=399.2.

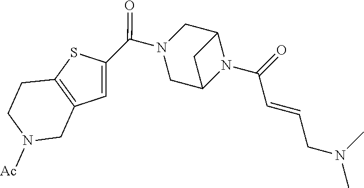 [SMILES: CC(=O)N1CCc2sc(C(=O)N3CC4CC(C3)N4C(=O)/C=C/CN(C)C)cc2C1]

Example 123: (E)-1-(3-(5-acetyl-4,5,6,7-tetrahydrothieno[3,2-c]pyridine-2-carbonyl)-3,6-diazabicyclo[3.1.1]heptan-6-yl)-4-(dimethylamino)but-2-en-1-one (XS190-69). Example 123 was synthesized following similar procedure for preparing example 79. White solid, 22% yield ¹H NMR (400 MHz, Methanol-d₄) δ 7.26 (d, J=10.6 Hz, 1H), 6.67-6.53 (m, 1H), 6.46 (dd, J=14.7, 6.7 Hz, 1H), 4.66 (s, 1H), 4.53-4.36 (m, 3H), 4.31-3.94 (m, 2H), 3.93-3.64 (m, 6H), 2.84-2.78 (m, 1H), 2.78-2.62 (m, 8H), 2.14-1.99 (m, 3H), 1.58 (d, J=9.2 Hz, 1H).MS (ESI) [M+H]⁺=417.3.

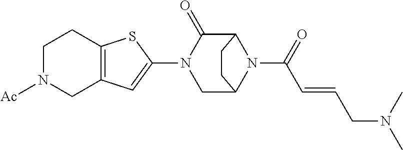 [SMILES: CC(=O)N1CCc2sc(N3CC4CCC(C3=O)N4C(=O)/C=C/CN(C)C)cc2C1]

Example 124: (E)-3-(5-acetyl-4,5,6,7-tetrahydrothieno[3,2-c]pyridin-2-yl)-8-(4-(dimethylamino)but-2-enoyl)-3,8-diazabicyclo[3.2.1]octan-2-one (XS190-70). Example 124 was synthesized following similar procedure for preparing example 79. White solid, 43% yield ¹H NMR (400 MHz, Methanol-d₄) δ 6.94-6.63 (m, 2H), 6.47-6.32 (m, 1H), 5.10-4.94 (m, 1H), 4.46 (s, 2H), 4.03-3.83 (m, 3H), 3.81-3.75 (m, 1H), 3.75-3.69 (m, 1H), 3.69-3.53 (m, 2H), 2.85-2.78 (m, 6H), 2.77-2.60 (m, 2H), 2.40-2.15 (m, 2H), 2.11 (s, 3H), 2.07 (s, 1H), 2.03-1.86 (m, 1H).MS (ESI) [M+H]⁺=417.3.

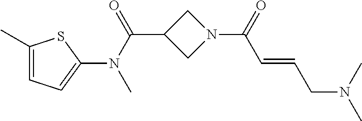

Example 125: (E)-1-(4-(dimethylamino)but-2-enoyl)-N-methyl-N-(5-methylthiophen-2-yl)azetidine-3-carboxamide (XS190-75). Example 125 was synthesized following similar procedure for preparing example 79. White solid, 26% yield ¹H NMR (400 MHz, Methanol-d₄) δ 6.84-6.70 (m, 1H), 6.51 (d, J=15.1 Hz, 1H), 6.44 (s, 2H), 4.52-4.39 (m, 2H), 4.23 (t, J=9.7 Hz, 1H), 4.19-4.04 (m, 3H), 3.60-3.48 (m, 1H), 3.10 (s, 9H), 2.32 (s, 3H).MS (ESI) [M+H]⁺=322.4.

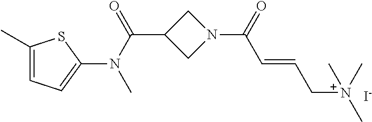

Example 126: (E)-N,N,N-trimethyl-4-(3-(methyl(5-methylthiophen-2-yl)carbamoyl)azetidin-1-yl)-4-oxobut-2-en-1-aminium (XS190-75-2). Example 126was synthesized following similar procedure for preparing example 79. White solid, 15% yield ¹H NMR (400 MHz, Methanol-d₄) δ 6.79-6.68 (m, 1H), 6.67 (d, 1=3.6 Hz, 1H), 6.60-6.55 (m, 1H), 6.43 (d, J=15.1 Hz, 1H), 4.41-4.33 (m, 1H), 4.17 (t, J=8.9 Hz, 1H), 4.11-4.02 (m, 3H), 3.88 (t, J=9.7 Hz, 1H), 3.71-3.59 (m, 1H), 3.18 (s, 3H), 3.06 (s, 9H), 2.39 (s, 3H).MS (ESI) [M+H]⁺=322.4.

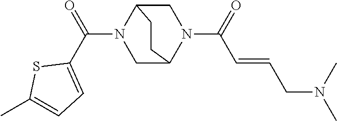

Example 127: (E)-4-(dimethylamino)-1-(5-(5-methylthiophene-2-carbonyl)-2,5-diazabicyclo[2.2.2]octan-2-yl)but-2-en-1-one (XS190-77). Example 127 was synthesized following similar procedure for preparing example 79. White solid, 46% yield ¹H NMR (400 MHz, Methanol-d₄) δ 7.24 (d, J=75.4 Hz, 1H), 6.91-6.54 (m, 3H), 4.73 (d, J=24.1 Hz, 1H), 4.62-4.33 (m, 1H), 4.11-3.71 (m, 4H), 3.69-3.49 (m, 2H), 2.81 (s, 6H), 2.41 (s, 3H), 2.16-1.70 (m, 4H).MS (ESI) [M+H]⁻=348.4.

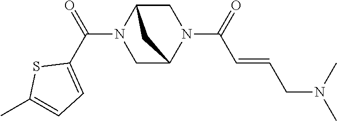

Example 128: (E)-4-(dimethylamino)-1-((1S,4S)-5-(5-methylthiophene-2-carbonyl)-2,5-diazabicyclo[2.2.1]heptan-2-yl)but-2-en-1-one (XS190-80). Example 128 was synthesized following similar procedure for preparing example 79. White solid, 42% yield ¹H NMR (400 MHz, Methanol-d₄) δ 7.31-7.14 (m, 1H), 6.80-6.34 (m, 3H), 4.86-4.78 (m, 2H), 3.80 (h, J=18.8, 15.5 Hz, 3H), 3.67-3.31 (m, 3H), 2.73 (d, J=7.4 Hz, 6H), 2.34 (s, 3H), 2.03-1.77 (m, 2H).MS (ESI) [M+H]⁺=334.1.

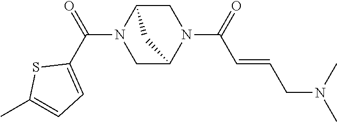

Example 129: (E)-4-(dimethylamino)-1-((1R,4R)-5-(5-methylthiophene-2-carbonyl)-2,5-diazabicyclo[2.2.1]heptan-2-yl)but-2-en-1-one (XS190-81). Example 129 was synthesized following similar procedure for preparing example 79. White solid, 59% yield ¹H NMR (400 MHz, Methanol-d4) δ 7.35-7.18 (m, 1H), 6.90-6.40 (m, 3H), 4.87-4.80 (m, 2H), 3.98-3.71 (m, 3H), 3.72-3.32 (m, 3H), 2.82-2.70 (m, 6H), 2.36 (s, 3H), 2.03-1.78 (m, 2H).MS (ESI) [M+H]⁻=334.1.

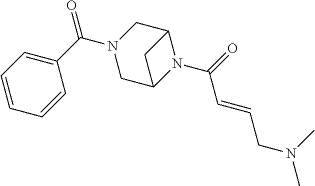 [SMILES: CN(C)C/C=C/C(=O)N1C2CC1CN(C(=O)c1ccccc1)C2]

Example 130: (E)-1-(3-benzoyl-3,6-diazabicyclo[3.1.1]heptan-6-yl)-4-(dimethylamino)but-2-en-1-one (XS190-126). Example 130 was synthesized following similar procedure for preparing example 79. White solid, 57% yield ¹H NMR (400 MHz, Methanol-d₄) δ 7.50-7.33 (m, 5H), 6.83-6.66 (m, 1H), 6.65-6.40 (m, 1H), 4.81-4.29 (m, 2H), 4.11-3.55 (m, 6H), 2.92-2.83 (m, 6H), 2.81-2.71 (m, 1H), 1.71 (t, J=8.2 Hz, 1H).MS (ESI) [M+H]⁺=314.2.

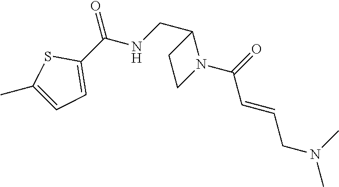

Example 131 (E)-N-((1-(4-(dimethylamino)but-2-enoyl)azetidin-2-yl)methyl)-5-methylthiophene-2-carboxamide (XS190-127). Example 131 was synthesized following similar procedure for preparing example 79. White solid, 34% yield ¹H NMR (400 MHz, Methanol-d₄) δ 7.55-7.42 (m, 1H), 6.85-6.63 (m, 2H), 6.59-6.41 (m, 1H), 4.78-4.57 (m, 1H), 4.24 (t, J=7.8 Hz, 1H), 4.02-3.89 (m, 2H), 3.88-3.73 (m, 2H), 3.69-3.50 (m, 1H), 2.88 (d, J=11.0 Hz, 6H), 2.59-2.41 (m, 4H), 2.24-2.08 (m, 1H).MS (ESI) [M+H]⁺=322.2.

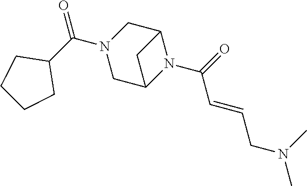 [SMILES: CN(C)C/C=C/C(=O)N1C2CC1CN(C(=O)C1CCCC1)C2]

Example 132 (E)-1-(3-(cyclopentanecarbonyl)-3,6-diazabicyclo[3.1.1]heptan-6-yl)-4-(dimethylamino)but-2-en-1-one (XS190-128). Example 132 was synthesized following similar procedure for preparing example 79. White solid, 34% yield ¹H NMR (400 MHz, Methanol-d₄) δ 6.78-6.63 (m, 1H), 6.61-6.48 (m, 1H), 4.75 (s, 1H), 4.52 (s, 1H), 4.20-3.66 (m, 6H), 3.05-2.94 (m, 1H), 2.88 (d, J=2.7 Hz, 6H), 2.83-2.74 (m, 1H), 1.95-1.80 (m, 2H), 1.80-1.55 (m, 7H).MS (ESI) [M+H]⁻=306.2.

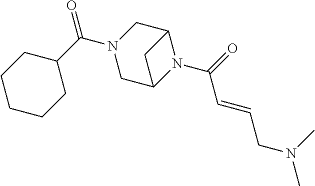 [SMILES: CN(C)C/C=C/C(=O)N1C2CC1CN(C(=O)C1CCCCC1)C2]

Example 133 (E)-1-(3-(cyclohexanecarbonyl)-3,6-diazabicyclo[3.1.1]heptan-6-yl)-4-(dimethylamino)but-2-en-1-one (XS190-129). Example 133 was synthesized following similar procedure for preparing example 79. White solid, 45% yield ¹H NMR (400 MHz, Methanol-d₄) δ 6.84-6.66 (m, 1H), 6.62-6.46 (m, 1H), 4.75 (s, 1H), 4.52 (s, 1H), 4.24-3.59 (m, 6H), 2.89 (d, J=5.2 Hz, 6H), 2.84-2.74 (m, 1H), 2.63-2.47 (m, 1H), 1.86-1.58 (m, 6H), 1.54-1.13 (m, 5H).MS (ESI) [M+H]⁻=320.2.

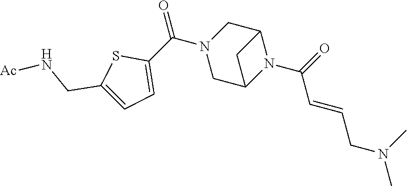 [SMILES: CC(=O)NCc1ccc(C(=O)N2CC3CC(C2)N3C(=O)/C=C/CN(C)C)s1]

Example 134 (E)-N-((5-(6-(4-(dimethylamino)but-2-enoyl)-3,6-diazabicyclo[3.1.1]heptane-3-carbonyl)thiophen-2-yl)methyl)acetamide (XS190-130). Example 134 was synthesized following similar procedure for preparing example 79. White solid, 22% yield ¹H NMR (400 MHz, Methanol-d₄) δ 7.53-7.40 (m, 1H), 7.01 (d, J=3.3 Hz, 1H), 6.84-6.70 (m, 1H), 6.59 (d, J=15.1 Hz, 1H), 4.80 (s, 1H), 4.54 (s, 3H), 4.47-3.86 (m, 6H), 2.98-2.76 (m, 7H), 1.99 (s, 3H), 1.74 (d, J=9.2 Hz, 1H).MS (ESI) [M+H]⁺=391.2.

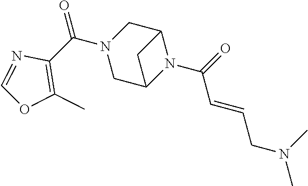 [SMILES: Cc1ocnc1C(=O)N1CC2CC(C1)N2C(=O)/C=C/CN(C)C]

Example 135 (E)-4-(dimethylamino)-1-(3-(5-methyloxazole-4-carbonyl)-3,6-diazabicyclo[3.1.1]heptan-6-yl)but-2-en-1-one (XS190-137). Example 135 was synthesized following similar procedure for preparing example 79. White solid, 47% yield ¹H NMR (400 MHz, Methanol-d₄) δ 8.05 (d, J=3.4 Hz, 1H), 6.85-6.73 (m, 1H), 6.67-6.54 (m, 1H), 4.77 (s, 1H), 4.58 (d, J=31.8 Hz, 1H), 4.49-4.23 (m, 2H), 4.09-3.88 (m, 4H), 2.93 (d, J=3.0 Hz, 6H), 2.85 (d, J=5.8 Hz, 1H), 2.54 (s, 3H), 1.80-1.71 (m, 1H).MS (ESI) [M+H]⁺=319.2.

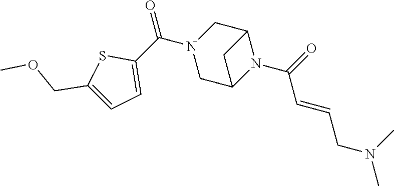

Example 136 (E)-4-(dimethylamino)-1-(3-(5-(methoxymethyl)thiophene-2-carbonyl)-3,6-diazabicyclo[3.1.1]heptan-6-yl)but-2-en-1-one (XS190-138). Example 136 was synthesized following similar procedure for preparing example 79. White solid, 38% yield ¹H NMR (400 MHz, Methanol-d₄) δ 7.49 (d, J=3.8 Hz, 1H), 7.05 (d, J=3.8 Hz, 1H), 6.83-6.71 (m, 1H), 6.59 (d, J=15.2 Hz, 1H), 4.81 (s, 1H), 4.63 (s, 3H), 4.47-3.82 (m, 6H), 3.39 (s, 3H), 2.97-2.78 (m, 7H), 1.74 (d, J=9.2 Hz, 1H).MS (ESI) [M+H]⁺=364.2.

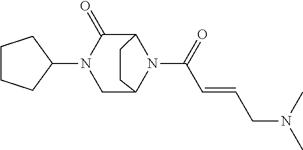 [SMILES: CN(C)C/C=C/C(=O)N1C2CCC1C(=O)N(C1CCCC1)C2]

Example 137 (E)-3-cyclopentyl-8-(4-(dimethylamino)but-2-enoyl)-3,8-diazabicyclo[3.2.1]octan-2-one (XS190-144). Example 137 was synthesized following similar procedure for preparing example 28. White solid, 19% yield ¹H NMR (400 MHz, Methanol-d₄) δ 6.81-6.44 (m, 2H), 4.75-4.28 (m, 3H), 3.78-3.63 (m, 2H), 3.41-3.25 (m, 1H), 2.95-2.80 (m, 1H), 2.66 (s, 6H), 2.15-1.75 (m, 3H), 1.70-1.54 (m, 2H), 1.47 (s, 3H), 1.41-1.21 (m, 4H).MS (ESI) [M+H]⁻=306.2.

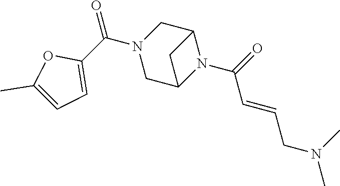

Example 138 (E)-4-(dimethylamino)-1-(3-(5-methylfuran-2-carbonyl)-3,6-diazabicyclo[3.1.1]heptan-6-yl)but-2-en-1-one (XS190-157). Example 138 was synthesized following similar procedure for preparing example 104. White solid, 35% yield ¹H NMR (400 MHz, Methanol-d₄) δ 7.04 (d, J=3.5 Hz, 1H), 6.78-6.65 (m, 1H), 6.58 (d, J=15.3 Hz, 1H), 6.20 (s, 1H), 4.79 (s, 1H), 4.56 (s, 1H), 4.47-3.79 (m, 6H), 2.90-2.75 (m, 7H), 2.34 (s, 3H), 1.69 (d, J=9.2 Hz, 1H).MS (ESI) [M+H]⁺=318.2.

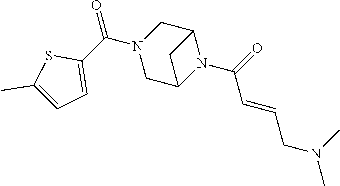

Example 139 (E)-4-(dimethylamino)-1-(3-((5-methylthiophen-2-yl)methyl)-3,6-diazabicyclo[3.1.1]heptan-6-yl)but-2-en-1-one (XS190-158). Example 139 was synthesized following similar procedure for preparing example 79. White solid, 23% yield ¹H NMR (400 MHz, Methanol-d₄) δ 7.14 (d, J=3.5 Hz, 1H), 6.85-6.70 (m, 2H), 6.53 (d, J=15.2 Hz, 1H), 4.82-4.53 (m, 4H), 3.96 (d, J=7.1 Hz, 2H), 3.90-3.61 (m, 4H), 2.83 (s, 2H), 2.52 (s, 3H), 2.07 (d, J=10.2 Hz, 1H).MS (ESI) [M+H]⁺=320.2.

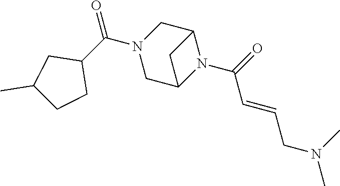

Example 140 (E)-4-(dimethylamino)-1-(3-(3-methylcyclopentane-1-carbonyl)-3,6-diazabicyclo[3.1.1]heptan-6-yl)but-2-en-1-one (XS190-170). Example 40 was synthesized following similar procedure for preparing example 79. White solid, 25% yield ¹H NMR (400 MHz, Methanol-d₄) δ 6.66-6.54 (m, 1H), 6.47-6.36 (m, 1H), 4.62 (s, 1H), 4.38 (s, 1H), 4.02 (d, J=11.4 Hz, 1H), 3.86-3.69 (m, 4H), 3.57 (d, J=10.1 Hz, 1H), 2.77-2.73 (m, 6H), 2.70-2.61 (m, 1H), 2.02-1.59 (m, 6H), 1.49 (d, J=9.4 Hz, 1H), 1.24-1.03 (m, 2H), 0.92-0.86 (m, 3H).MS (ESI) [M+H]⁺=320.2.

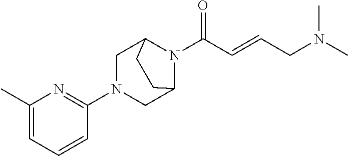

Example 141 (E)-8-(4-(dimethylamino)but-2-enoyl)-3-(6-methylpyridin-2-yl)-3,8-diazabicyclo[3.2.1]octan-2-one (XS190-181). Example 141 was synthesized following similar procedure for preparing example 28. White solid, 29% yield ¹H NMR (400 MHz, Methanol-d₄) δ 7.84-7.60 (m, 1H), 7.46-7.07 (m, 1H), 6.99-6.72 (m, 2H), 6.72-6.47 (m, 1H), 4.97 (dd, J=42.5, 7.7 Hz, 1H), 4.84-4.70 (m, 1H), 3.96-3.83 (m, 2H), 3.81-3.70 (m, 1H), 2.91-2.75 (m, 6H), 2.47 (d, J=37.5 Hz, 3H), 2.39-2.15 (m, 3H), 2.14-1.79 (m, 1H).MS (ESI) [M+H]⁺=329.2.

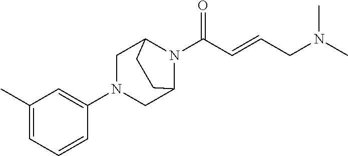

Example 142 (E)-8-(4-(dimethylamino)but-2-enoyl)-3-(m-tolyl)-3,8-diazabicyclo[3.2.1]octan-2-one (XS190-182). Example 142 was synthesized following similar procedure for preparing example 28. White solid, 34% yield ¹H NMR (400 MHz, Methanol-d₄) δ 7.20 (t, J=7.7 Hz, 1H), 7.05 (d, J=7.5 Hz, 1H), 6.98 (s, 1H), 6.93 (d, J=7.8 Hz, 1H), 6.90-6.66 (m, 2H), 4.98-4.88 (m, 1H), 4.83-4.70 (m, 1H), 3.95-3.75 (m, 3H), 3.39 (dd, J=21.6, 11.8 Hz, 1H), 2.81 (s, 6H), 2.43-1.96 (m, 7H).MS (ESI) [M+H]⁺=328.2.

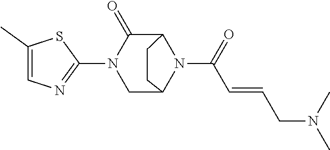

Example 143 (E)-8-(4-(dimethylamino)but-2-enoyl)-3-(5-methylthiazol-2-yl)-3,8-diazabicyclo[3.2.1]octan-2-one (XS190-183). Example 143 was synthesized following similar procedure for preparing example 28. White solid, 33% yield ¹H NMR (400 MHz, Methanol-d₄) δ 6.87 (s, 1H), 6.77-6.46 (m, 2H), 4.84-4.70 (m, 2H), 3.90-3.60 (m, 4H), 2.65 (s, 6H), 2.22-1.91 (m, 6H), 1.85-1.58 (m, 1H).MS (ESI) [M+H]⁺=335.2.

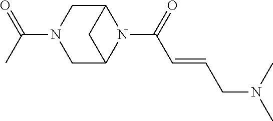 [SMILES: CC(=O)N1CC2CC(C1)N2C(=O)/C=C/CN(C)C]

Example 144 (E)-1-(3-acetyl-3,6-diazabicyclo[3.1.1]heptan-6-yl)-4-(dimethylamino)but-2-en-1-one (XS190-186). Example 144 was synthesized following similar procedure for preparing example 79. White solid, 19% yield ¹H NMR (400 MHz, Methanol-d₄) δ 6.67-6.54 (m, 1H), 6.46-6.33 (m, 1H), 4.61 (s, 1H), 4.38 (s, 1H), 3.84-3.68 (m, 4H), 3.66-3.52 (m, 2H), 2.75 (d, J=5.3 Hz, 6H), 2.70-2.58 (m, 1H), 1.94 (s, 3H), 1.57-1.45 (m, 1H).MS (ESI) [M+H]⁺=252.2.

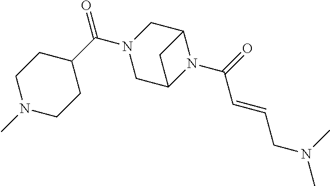

Example 145 (E)-4-(dimethylamino)-1-(3-(1-methylpiperidine-4-carbonyl)-3,6-diazabicyclo[3.1.1]heptan-6-yl)but-2-en-1-one (XS197-3). Example 145 was synthesized following similar procedure for preparing example 79. White solid, 17% yield ¹H NMR (400 MHz, Methanol-d4) δ 6.76-6.60 (m, 1H), 6.51 (d, J=15.4 Hz, 1H), 4.78-4.34 (m, 3H), 4.10-3.80 (m, 5H), 3.78-3.34 (m, 4H), 2.90-2.87 (m, 6H), 2.82-2.76 (m, 9H), 1.85 (d, J=9.5 Hz, 1H).MS (ESI) [M+H]⁺=335.2.

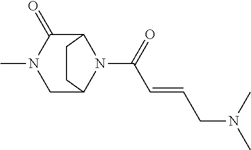

Example 146 (E)-8-(4-(dimethylamino)but-2-enoyl)-3-methyl-3,8-diazabicyclo[3.2.1]octan-2-one (XS197-4). Example 146 was synthesized following similar procedure for preparing example 28. White solid, 13% yield ¹H NMR (400 MHz, Methanol-d₄) δ 6.88-6.62 (m, 2H), 4.82-4.49 (m, 2H), 3.84 (d, J=7.4 Hz, 2H), 3.66-3.41 (m, 2H), 2.77 (d, J=6.3 Hz, 6H), 2.72 (s, 3H), 2.28-1.70 (m, 4H).MS (ESI) [M+H]⁺=252.2.

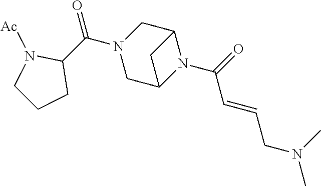 [SMILES: CC(=O)N1CCCC1C(=O)N1CC2CC(C1)N2C(=O)/C=C/CN(C)C]

Example 147 (E)-8-(4-(dimethylamino)but-2-enoyl)-3-methyl-3,8-diazabicyclo[3.2.1]octan-2-one (XS197-5). Example 147 was synthesized following similar procedure for preparing example 79. White solid, 25% yield ¹H NMR (400 MHz, Methanol-d₄) δ 6.73-6.56 (m, 1H), 6.54-6.35 (m, 1H), 4.71-4.51 (m, 2H), 4.42 (s, 1H), 4.12-3.71 (m, 4H), 3.70-3.38 (m, 4H), 2.83-2.73 (m, 6H), 2.73-2.63 (m, 1H), 2.25-2.08 (m, 1H), 2.05-1.79 (m, 5H), 1.78-1.46 (m, 2H).MS (ESI) [M+H]⁺=349.2.

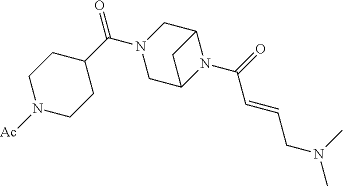 [SMILES: CC(=O)N1CCC(C(=O)N2CC3CC(C2)N3C(=O)/C=C/CN(C)C)CC1]

Example 148 (E)-1-(3-(1-acetylpiperidine-4-carbonyl)-3,6-diazabicyclo[3.1.1]heptan-6-yl)-4-(dimethylamino)but-2-en-1-one (XS197-6). Example 148 was synthesized following similar procedure for preparing example 79. White solid, 23% yield ¹H NMR (400 MHz, Methanol-d4) δ 6.68-6.53 (m, 1H), 6.47-6.35 (m, 1H), 4.62 (d, J=12.9 Hz, 1H), 4.38 (s, 2H), 4.11-3.63 (m, 6H), 3.63-3.51 (m, 3H), 2.74 (d, J=6.9 Hz, 6H), 2.68-2.48 (m, 2H), 2.01 (d, J=48.1 Hz, 3H), 1.73-1.28 (m, 5H).MS (ESI) [M+H]⁺=363.2.

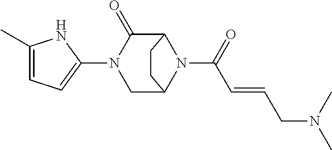

Example 149 (E)-8-(4-(dimethylamino)but-2-enoyl)-3-(5-methyl-1H-pyrrol-2-yl)-3,8-diazabicyclo[3.2.1]octan-2-one (XS197-14). Example 149 was synthesized following similar procedure for preparing example 28. White solid, 34% yield ¹H NMR (400 MHz, Methanol-d₄) δ 7.55-6.56 (m, 2H), 6.49 (d, J=15.2 Hz, 2H), 5.83 (s, 1H), 4.71 (s, 1H), 4.48 (s, 1H), 3.83 (d, J=6.5 Hz, 4H), 2.84-2.68 (m, 8H), 2.15 (s, 3H), 1.59 (d, J=9.1 Hz, 1H), 1.32-1.16 (m, 1H).MS (ESI) [M+H]⁺=317.2.

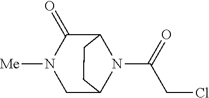 [SMILES: CN1CC2CCC(C1=O)N2C(=O)CCl]

Example 150 (XS197-29). 8-(2-chloroacetyl)-3-methyl-3,8-diazabicyclo[3.2.1]octan-2-one Example 150 was synthesized following similar procedure for preparing example 18. White solid, 33% yield ¹H NMR (400 MHz, Methanol-d₄) δ 4.30-4.06 (m, 2H), 3.75-3.52 (m, 2H), 3.20-3.00 (m, 2H), 2.80 (s, 3H), 2.34-1.76 (m, 4H).MS (ESI) [M+H]⁺=217.1.

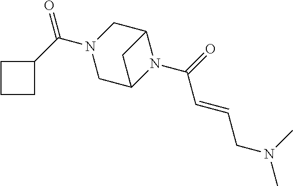 [SMILES: CN(C)C/C=C/C(=O)N1C2CC1CN(C(=O)C1CCC1)C2]

Example 151 (E)-1-(3-(cyclobutanecarbonyl)-3,6-diazabicyclo[3.1.1]heptan-6-yl)-4-(dimethylamino)but-2-en-1-one (XS197-38). Example 151 was synthesized following similar procedure for preparing example 79. White solid, 23% yield ¹H NMR (400 MHz, Methanol-d₄) δ 6.55 (dt, J=14.7, 7.1 Hz, 1H), 6.41 (d, J=15.2 Hz, 1H), 4.64-4.52 (m, 1H), 4.40-4.27 (m, 1H), 3.90-3.72 (m, 3H), 3.67-3.44 (m, 3H), 3.32-3.22 (m, 1H), 2.72 (s, 6H), 2.61 (t, J=7.6 Hz, 1H), 2.14-1.96 (m, 4H), 1.91-1.81 (m, 1H), 1.73-1.57 (m, 1H), 1.46 (d, J=9.2 Hz, 1H).MS (ESI) [M+H]⁺=292.2.

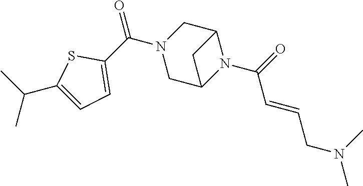 [SMILES: CC(C)c1ccc(C(=O)N2CC3CC(C2)N3C(=O)/C=C/CN(C)C)s1]

Example 152 (E)-4-(dimethylamino)-1-(3-(5-isopropylthiophene-2-carbonyl)-3,6-diazabicyclo[3.1.1]heptan-6-yl)but-2-en-1-one (XS197-49). Example 152 was synthesized following similar procedure for preparing example 79. White solid, 46% yield ¹H NMR (400 MHz, Methanol-d₄) δ 7.48 (d, J=3.9 Hz, 1H), 6.91 (d, J=3.9 Hz, 1H), 6.85-6.72 (m, 1H), 6.62 (d, J=15.3 Hz, 1H), 4.82 (s, 1H), 4.65-4.51 (m, 1H), 4.48-3.91 (m, 6H), 3.22 (p, J=6.9 Hz, 1H), 2.98-2.78 (m, 7H), 1.74 (d, J=9.2 Hz, 1H), 1.35 (d, J=6.9 Hz, 6H).MS (ESI) [M+H]⁻=362.2.

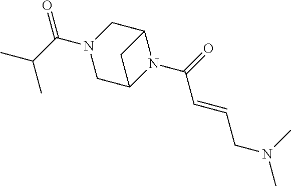 [SMILES: CC(C)C(=O)N1CC2CC(C1)N2C(=O)/C=C/CN(C)C]

Example 153 (E)-4-(dimethylamino)-1-(3-isobutyryl-3,6-diazabicyclo[3.1.1]heptan-6-yl)but-2-en-1-one (XS197-50). Example 153 was synthesized following similar procedure for preparing example 79. White solid, 27% yield ¹H NMR (400 MHz, Methanol-d₄) δ 6.71-6.58 (m, 1H), 6.55-6.41 (m, 1H), 4.68 (s, 1H), 4.44 (s, 1H), 3.91-3.81 (m, 3H), 3.70-3.56 (m, 3H), 3.14 (q, J=7.4 Hz, 1H), 2.81 (s, 6H), 2.74-2.68 (m, 1H), 1.59-1.53 (m, 1H), 1.08-0.96 (m, 6H).MS (ESI) [M+H]⁺=280.2.

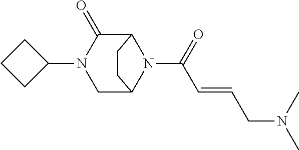 [SMILES: CN(C)C/C=C/C(=O)N1C2CCC1C(=O)N(C1CCC1)C2]

Example 154 (E)-3-cyclobutyl-8-(4-(dimethylamino)but-2-enoyl)-3,8-diazabicyclo[3.2.1]octan-2-one (XS197-70). Example 154 was synthesized following similar procedure for preparing example 79. White solid, 21% yield ¹H NMR (400 MHz, Methanol-d4) δ 6.83-6.55 (m, 2H), 4.81-4.43 (m, 3H), 3.88-3.70 (m, 2H), 3.52-3.37 (m, 1H), 3.15-3.07 (m, 1H), 2.75 (d, J=6.0 Hz, 6H), 2.25-1.83 (m, 7H), 1.83-1.65 (m, 1H), 1.62-1.46 (m, 2H).MS (ESI) [M+H]⁺=292.2.

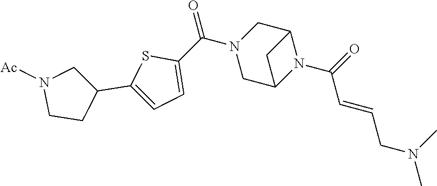 [SMILES: CC(=O)N1CCC(c2ccc(C(=O)N3CC4CC(C3)N4C(=O)/C=C/CN(C)C)s2)C1]

Example 155 (E)-1-(3-(5-(1-acetylpyrrolidin-3-yl)thiophene-2-carbonyl)-3,6-diazabicyclo[3.1.1]heptan-6-yl)-4-(dimethylamino)but-2-en-1-one (XS197-71). Example 155 was synthesized following similar procedure for preparing example 79. White solid, 26% yield H NMR (400 MHz, Methanol-d₄) δ 7.41 (s, 1H), 6.98-6.88 (m, 1H), 6.74-6.61 (m, 1H), 6.55-6.42 (m, 1H), 4.72 (s, 1H), 4.47 (s, 1H), 4.11-3.79 (m, 6H), 3.71-3.52 (m, 3H), 3.40-3.29 (m, 1H), 3.14 (q, J=7.4 Hz, 1H), 2.81 (s, 6H), 2.78-2.74 (m, 1H), 2.43-2.25 (m, 1H), 2.13-1.93 (m, 4H), 1.65 (d, J=9.2 Hz, 1H).MS (ESI) [M+H]⁺=431.2.

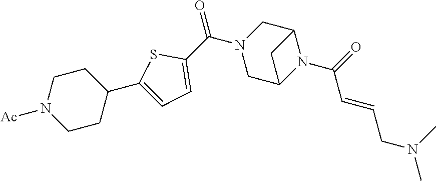 [SMILES: CC(=O)N1CCC(c2ccc(C(=O)N3CC4CC(C3)N4C(=O)/C=C/CN(C)C)s2)CC1]

Example 156 (E)-1-(3-(5-(1-acetylpiperidin-4-yl)thiophene-2-carbonyl)-3,6-diazabicyclo[3.1.1]heptan-6-yl)-4-(dimethylamino)but-2-en-1-one (XS197-72). Example 156 was synthesized following similar procedure for preparing example 79. White solid, 25% yield ¹H NMR (400 MHz, Methanol-d₄) δ 7.37 (d, J=3.9 Hz, 1H), 6.83 (d, J=4.0 Hz, 1H), 6.70-6.58 (m, 1H), 6.48 (d, J=15.3 Hz, 1H), 4.69 (s, 1H), 4.47 (t, J=14.4 Hz, 2H), 4.35-4.07 (m, 1H), 4.02-3.73 (m, 6H), 3.18-3.00 (m, 2H), 2.78 (s, 6H), 2.73-2.59 (m, 2H), 2.05-1.90 (m, 5H), 1.64-1.38 (m, 3H).MS (ESI) [M+H]⁺=445.2.

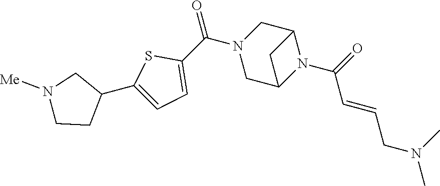 [SMILES: CN(C)C/C=C/C(=O)N1C2CC1CN(C(=O)c1ccc(C3CCN(C)C3)s1)C2]

Example 157 (E)-4-(dimethylamino)-1-(3-(5-(1-methylpyrrolidin-3-yl)thiophene-2-carbonyl)-3,6-diazabicyclo[3.1.1]heptan-6-yl)but-2-en-1-one (XS197-73). Example 157 was synthesized following similar procedure for preparing example 79. White solid, 20% yield ¹H NMR (400 MHz, Methanol-d₄) δ 7.46 (d, J=4.3 Hz, 1H), 7.02 (s, 1H), 6.77-6.64 (m, 1H), 6.53 (d, J=15.0 Hz, 1H), 4.74 (s, 1H), 4.51 (d, J=19.5 Hz, 1H), 4.39-4.12 (m, 1H), 4.09-3.95 (m, 2H), 3.93-3.72 (m, 5H), 3.68-3.49 (m, 1H), 3.21-3.08 (m, 1H), 2.95 (s, 3H), 2.83 (s, 6H), 2.81-2.70 (m, 2H), 2.67-2.45 (m, 1H), 2.35-2.07 (m, 1H), 1.67 (d, J=9.2 Hz, 1H).MS (ESI) [M+H]⁺=403.2.

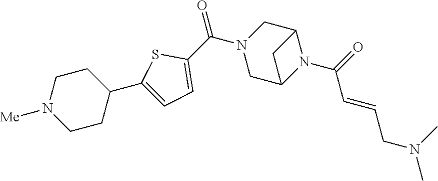 [SMILES: CN(C)C/C=C/C(=O)N1C2CC1CN(C(=O)c1ccc(C3CCN(C)CC3)s1)C2]

Example 158 (E)-4-(dimethylamino)-1-(3-(5-(1-methylpiperidin-4-yl)thiophene-2-carbonyl)-3,6-diazabicyclo[3.1.1]heptan-6-yl)but-2-en-1-one (XS197-74). Example 158 was synthesized following similar procedure for preparing example 79. White solid, 17% yield ¹H NMR (400 MHz, Methanol-d₄) δ 7.43 (d, J=4.1 Hz, 1H), 6.91 (d, J=3.9 Hz, 1H), 6.76-6.62 (m, 1H), 6.51 (d, J=15.1 Hz, 1H), 4.72 (s, 1H), 4.49 (d, J=18.1 Hz, 1H), 4.32-4.07 (m, 1H), 4.01 (d, J=11.4 Hz, 1H), 3.88-3.82 (m, 3H), 3.58-3.47 (m, 2H), 3.20-3.02 (m, 3H), 2.85-2.80 (m, 8H), 2.76-2.70 (m, 3H), 2.21 (d, J=14.4 Hz, 2H), 1.96-1.78 (m, 2H), 1.65 (d, J=9.2 Hz, 1H).MS (ESI) [M+H]⁺=417.2.

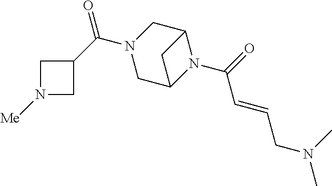 [SMILES: CN(C)C/C=C/C(=O)N1C2CC1CN(C(=O)C1CN(C)C1)C2]

Example 159 (E)-4-(dimethylamino)-1-(3-(1-methylazetidine-3-carbonyl)-3,6-diazabicyclo[3.1.1]heptan-6-yl)but-2-en-1-one (XS197-85). Example 159 was synthesized following similar procedure for preparing example 79. White solid, 19% yield ¹H NMR (400 MHz, Methanol-d₄) δ 6.70-6.56 (m, 1H), 6.48-6.36 (m, 1H), 4.70-4.61 (m, 1H), 4.47-4.32 (m, 3H), 4.11-3.98 (m, 2H), 3.94-3.79 (m, 4H), 3.75-3.65 (m, 2H), 2.86-2.79 (m, 4H), 2.79-2.75 (m, 6H), 2.73-2.64 (m, 1H), 1.57 (d, J=9.2 Hz, 1H).MS (ESI) [M+H]⁺=307.2.

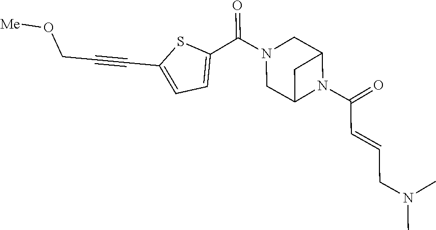 [SMILES: COCC#Cc1ccc(C(=O)N2CC3CC(C2)N3C(=O)/C=C/CN(C)C)s1]

Example 160 (E)-4-(dimethylamino)-1-(3-(5-(3-methoxyprop-1-yn-1-yl)thiophene-2-carbonyl)-3,6-diazabicyclo[3.1.1]heptan-6-yl)but-2-en-1-one (XS197-93). Example 160 was synthesized following similar procedure for preparing example 79. White solid, 44% yield ¹H NMR (400 MHz, Methanol-d₄) δ 7.39 (d, J=4.0 Hz, 1H), 7.12 (d, J=4.1 Hz, 1H), 6.69-6.58 (m, 1H), 6.53-6.39 (m, 1H), 4.69 (s, 1H), 4.46 (d, J=16.4 Hz, 1H), 4.34-4.18 (m, 3H), 4.10-3.92 (m, 2H), 3.87-3.79 (m, 3H), 3.29 (s, 3H), 2.84-2.65 (m, 7H), 1.62 (d, J=9.3 Hz, 1H).MS (ESI) [M+H]⁺=388.2.

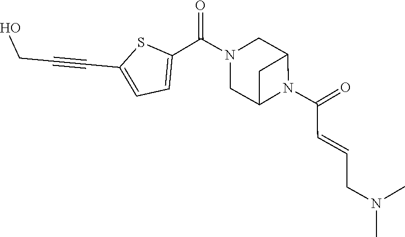 [SMILES: CN(C)C/C=C/C(=O)N1C2CC1CN(C(=O)c1ccc(C#CCO)s1)C2]

Example 161 (E)-4-(dimethylamino)-1-(3-(5-(3-hydroxyprop-1-yn-1-yl)thiophene-2-carbonyl)-3,6-diazabicyclo[3.1.1]heptan-6-yl)but-2-en-1-one (XS197-94). Example 161 was synthesized following similar procedure for preparing example 79. White solid, 28% yield ¹H NMR (400 MHz, Methanol-d4) δ 7.24 (d, J=3.9 Hz, 1H), 6.94 (d, J=4.0 Hz, 1H), 6.58-6.44 (m, 1H), 6.37-6.23 (m, 1H), 4.54 (s, 1H), 4.31 (d, J=14.2 Hz, 1H), 4.20-4.07 (m, 2H), 3.97-3.77 (m, 2H), 3.77-3.58 (m, 4H), 2.67-2.52 (m, 7H), 1.47 (d, J=9.2 Hz, 1H).MS (ESI) [M+H]⁻=374.1.

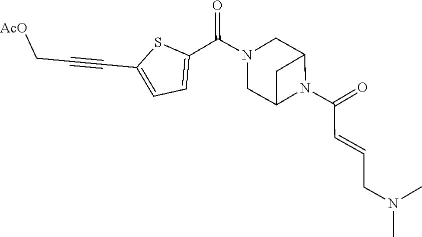 [SMILES: CC(=O)OCC#Cc1ccc(C(=O)N2CC3CC(C2)N3C(=O)/C=C/CN(C)C)s1]

Example 162 (E)-3-(5-(6-(4-(dimethylamino)but-2-enoyl)-3,6-diazabicyclo[3.1.1]heptane-3-carbonyl)thiophen-2-yl)prop-2-yn-1-yl acetate (XS197-96). Example 162 was synthesized following similar procedure for preparing example 79. White solid, 29% yield ¹H NMR (400 MHz, Methanol-d4) δ 7.45 (d, J=4.7 Hz, 1H), 7.19 (d, J=3.9 Hz, 1H), 6.75-6.64 (m, 1H), 6.51 (t, J=14.1 Hz, 1H), 4.92-4.88 (m, 1H), 4.73 (s, 1H), 4.51 (d, J=17.4 Hz, 1H), 4.33 (d, J=11.9 Hz, 1H), 4.19-3.98 (m, 2H), 3.96-3.80 (m, 4H), 2.90-2.71 (m, 7H), 2.03 (s, 3H), 1.67 (d, J=9.2 Hz, 1H).MS (ESI) [M+H]⁺=416.2.

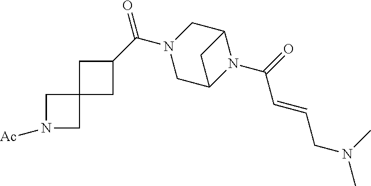 [SMILES: CC(=O)N1CC2(CC(C(=O)N3CC4CC(C3)N4C(=O)/C=C/CN(C)C)C2)C1]

Example 163 (E)-1-(3-(2-acetyl-2-azaspiro[3.3]heptane-6-carbonyl)-3,6-diazabicyclo[3.1.1]heptan-6-yl)-4-(dimethylamino)but-2-en-1-one (XS197-133). Example 163 was synthesized following similar procedure for preparing example 79. White solid, 22% yield ¹H NMR (400 MHz, Methanol-d₄) δ 6.73-6.61 (m, 1H), 6.48 (d, J=15.3 Hz, 1H), 4.67 (s, 1H), 4.46 (d, J=11.9 Hz, 1H), 4.18 (s, 1H), 4.05 (d, J=4.1 Hz, 1H), 3.99-3.93 (m, 2H), 3.87 (d, J=7.2 Hz, 2H), 3.80 (d, J=8.3 Hz, 1H), 3.76-3.57 (m, 3H), 2.83 (d, J=3.3 Hz, 6H), 2.78-2.68 (m, 2H), 2.45-2.27 (m, 4H), 1.77 (d, J=6.7 Hz, 3H), 1.61-1.54 (m, 1H).MS (ESI) [M+H]⁺=375.2.

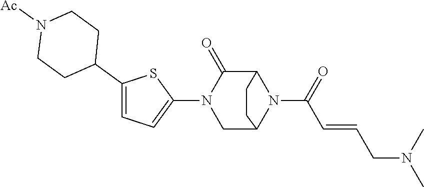 [SMILES: CC(=O)N1CCC(c2ccc(N3CC4CCC(C3=O)N4C(=O)/C=C/CN(C)C)s2)CC1]

Example 164 (E)-3-(5-(1-acetylpiperidin-4-yl)thiophen-2-yl)-8-(4-(dimethylamino)but-2-enoyl)-3,8-diazabicyclo[3.2.1]octan-2-one (XS197-145). Example 164 was synthesized following similar procedure for preparing example 28. White solid, 25% yield ¹H NMR (400 MHz, Methanol-d4) δ 6.95-6.65 (m, 2H), 6.61 (d, J=3.9 Hz, 1H), 6.57-6.46 (m, 1H), 5.01 (d, J=38.0 Hz, 2H), 4.51 (d, J=13.2 Hz, 1H), 4.06-3.83 (m, 4H), 3.64 (dd, J=20.1, 11.7 Hz, 1H), 3.23-3.10 (m, 1H), 2.97 (t, J=13.4 Hz, 1H), 2.84 (d, J=5.2 Hz, 6H), 2.74-2.61 (m, 1H), 2.40-2.10 (m, 3H), 2.06 (s, 3H), 2.03-1.85 (m, 3H), 1.70-1.41 (m, 2H).MS (ESI) [M+H]⁺=445.2.

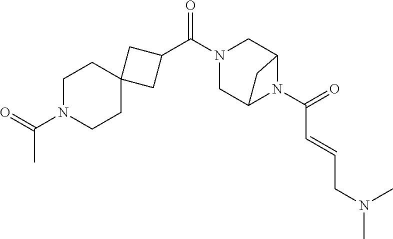 [SMILES: CC(=O)N1CCC2(CC1)CC(C(=O)N1CC3CC(C1)N3C(=O)/C=C/CN(C)C)C2]

Example 165 (E)-1-(3-(7-acetyl-7-azaspiro[3.5]nonane-2-carbonyl)-3,6-diazabicyclo[3.1.1]heptan-6-yl)-4-(dimethylamino)but-2-en-1-one (XS197-176). Example 165 was synthesized following similar procedure for preparing example 79. White solid, 18% yield ¹H NMR (400 MHz, Methanol-d₄) δ 6.81-6.68 (m, 1H), 6.56 (d, J=15.2 Hz, 1H), 4.75 (s, 1H), 4.53 (d, J=13.5 Hz, 1H), 4.07-3.86 (m, 3H), 3.73-3.67 (m, 2H), 3.59-3.52 (m, 1H), 3.52-3.43 (m, 2H), 3.43-3.37 (m, 2H), 2.90 (s, 6H), 2.82 (s, 3H), 2.15-2.05 (m, 6H), 1.73 (t, J=5.5 Hz, 1H), 1.69-1.62 (m, 2H), 1.58 (t, J=5.6 Hz, 1H), 1.50 (t, J=5.8 Hz, 1H).MS (ESI) [M+H]⁺=403.3.

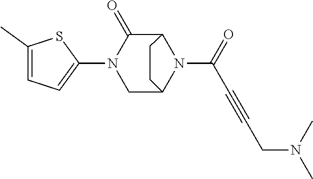

Example 166 8-(4-(dimethylamino)but-2-ynoyl)-3-(5-methylthiophen-2-yl)-3,8-diazabicyclo[3.2.1]octan-2-one (XS197-185). Example 166 was synthesized following similar procedure for preparing example 28. White solid, 39% yield ¹H NMR (400 MHz, Methanol-d₄) δ 6.59 (d, J=2.0 Hz, 2H), 5.12-4.91 (m, 2H), 4.41-4.34 (m, 2H), 4.14-4.00 (m, 1H), 3.81-3.65 (m, 1H), 3.11-3.01 (m, 6H), 2.41 (s, 3H), 2.40-2.32 (m, 1H), 2.31-2.16 (m, 2H), 2.12-2.00 (m, 1H).MS (ESI) [M+H]⁺=332.1.

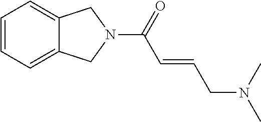 [SMILES: CN(C)C/C=C/C(=O)N1Cc2ccccc2C1]

Example 167 (E)-4-(dimethylamino)-1-(isoindolin-2-yl)but-2-en-1-one (XS197-186). Example 167 was synthesized following similar procedure for preparing example 79. White solid, 60% yield ¹H NMR (400 MHz, Methanol-d₄) δ 7.33-7.20 (m, 4H), 6.86-6.67 (m, 2H), 4.94 (s, 2H), 4.75 (s, 2H), 3.94 (d, J=6.2 Hz, 2H), 2.87 (s, 6H).MS (ESI) [M+H]⁺=231.2.

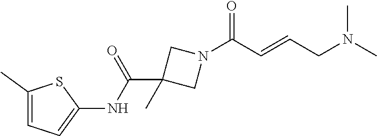

Example 168 (E)-1-(4-(dimethylamino)but-2-enoyl)-3-methyl-N-(5-methylthiophen-2-yl)azetidine-3-carboxamide (XS209-21). Example 168 was synthesized following similar procedure for preparing example 53. White solid, 43% yield ¹H NMR (400 MHz, Methanol-d₄) δ 6.77-6.65 (m, 1H), 6.55 (d, J=3.5 Hz, 1H), 6.52-6.42 (m, 2H), 4.65 (d, J=8.9 Hz, 1H), 4.35 (d, J=10.6 Hz, 1H), 4.12 (d, J=9.0 Hz, 1H), 3.99-3.85 (m, 3H), 2.89 (s, 6H), 2.37 (s, 3H), 1.65 (s, 3H).MS (ESI) [M+H]⁺=322.2.

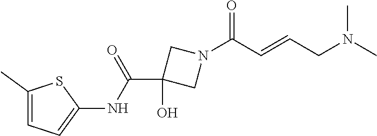

Example 169 (E)-1-(4-(dimethylamino)but-2-enoyl)-3-hydroxy-N-(5-methylthiophen-2-yl)azetidine-3-carboxamide (XS209-22). Example 169 was synthesized following similar procedure for preparing example 53. White solid, 29% yield ¹H NMR (400 MHz, Methanol-d₄) δ 6.84-6.71 (m, 1H), 6.68 (d, J=3.6 Hz, 1H), 6.55 (d, J=15.2 Hz, 2H), 4.71 (d, J=9.4 Hz, 1H), 4.46 (d, J=10.9 Hz, 1H), 4.29 (d, J=9.4 Hz, 1H), 4.05 (d, J=10.9 Hz, 1H), 3.97 (d, J 7.1 Hz, 2H), 2.92 (s, 6H), 2.41 (s, 3H).MS (ESI) [M+H]⁺=324.1.

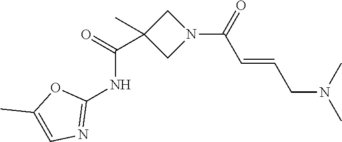

Example 170 (E)-1-(4-(dimethylamino)but-2-enoyl)-3-methyl-N-(5-methyloxazol-2-yl)azetidine-3-carboxamide (XS209-23). Example 170 was synthesized following similar procedure for preparing example 53. White solid, 38% yield ¹H NMR (400 MHz, Methanol-d₄) δ 6.81 (s, 1H), 6.79-6.66 (m, 1H), 6.50 (d, J=15.2 Hz, 1H), 4.71 (d, J=9.0 Hz, 1H), 4.39 (d, J=10.6 Hz, 1H), 4.14 (d, J=9.1 Hz, 1H), 4.00-3.89 (m, 3H), 2.91 (s, 6H), 2.32 (s, 3H), 1.68 (s, 3H).MS (ESI) [M+H]⁻=307.2.

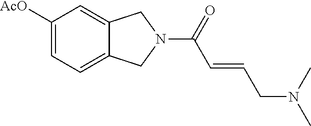 [SMILES: CC(=O)Oc1ccc2c(c1)CN(C(=O)/C=C/CN(C)C)C2]

Example 171 (E)-2-(4-(dimethylamino)but-2-enoyl)isoindolin-5-yl acetate (XS209-24). Example 171 was synthesized following similar procedure for preparing example 79. White solid, 55% yield ¹H NMR (400 MHz, Methanol-d4) δ 7.29 (t, J=9.3 Hz, 1H), 7.08-6.96 (m, 2H), 6.85-6.66 (m, 2H), 4.98-4.92 (m, 2H), 4.76 (d, J=3.8 Hz, 2H), 3.94 (d, J=5.6 Hz, 2H), 2.88 (s, 6H), 2.22 (s, 3H).MS (ESI) [M+H]⁺-289.1.

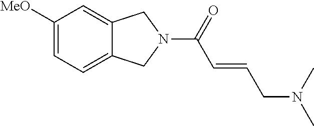 [SMILES: COc1ccc2c(c1)CN(C(=O)/C=C/CN(C)C)C2]

Example 172 (E)-4-(dimethylamino)-1-(5-methoxyisoindolin-2-yl)but-2-en-1-one (XS209-25). Example 172 was synthesized following similar procedure for preparing example 79. White solid, 45% yield ¹H NMR (400 MHz, Methanol-d₄) δ 7.12 (t, J=9.0 Hz, 1H), 6.96-6.82 (m, 2H), 6.77-6.68 (m, 2H), 4.96-4.89 (m, 2H), 4.76-4.69 (m, 2H), 4.24-3.94 (m, 2H), 3.24-2.80 (m, 9H).MS (ESI) [M+H]⁻=261.1.

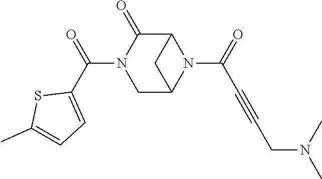

Example 173 4-(dimethylamino)-1-(3-(5-methylthiophene-2-carbonyl)-3,6-diazabicyclo[3.1.1]heptan-6-yl)but-2-yn-1-one (XS209-26). Example 174 was synthesized following similar procedure for preparing example 79. White solid, 29% yield ¹H NMR (400 MHz, Methanol-d₄) δ 7.43 (d, J=4.0 Hz, 1H), 6.82 (d, J=3.8 Hz, 1H), 4.72 (s, 1H), 4.59 (s, 1H), 4.31 (s, 3H), 4.25-3.83 (m, 3H), 2.97 (s, 6H), 2.87-2.74 (m, 1H), 2.49 (s, 3H), 1.73 (d, J=9.2 Hz, 1H).MS (ESI) [M+H]⁺=332.1.

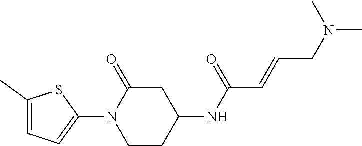

Example 174 (E)-4-(dimethylamino)-N-(1-(5-methylthiophen-2-yl)-2-oxopiperidin-4-yl)but-2-enamide (XS209-27). Example 174 was synthesized following similar procedure for preparing example 28. White solid, 27% yield ¹H NMR (400 MHz, Methanol-d₄) δ 6.81-6.66 (m, 1H), 6.63-6.51 (m, 2H), 6.37 (d, J=15.3 Hz, 1H), 4.37-4.24 (m, 1H), 3.96-3.76 (m, 4H), 2.89 (s, 7H), 2.58-2.46 (m, 1H), 2.38 (s, 3H), 2.32-2.21 (m, 1H), 2.11-2.00 (m, 1H).MS (ESI) [M+H]⁻=322.2.

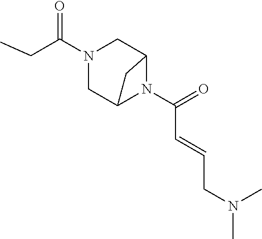 [SMILES: CCC(=O)N1CC2CC(C1)N2C(=O)/C=C/CN(C)C]

Example 175 (E)-4-(dimethylamino)-1-(3-propionyl-3,6-diazabicyclo[3.1.1]heptan-6-yl)but-2-en-1-one (XS209-28). Example 175 was synthesized following similar procedure for preparing example 79. White solid, 21% yield ¹H NMR (400 MHz, Methanol-d₄) δ 6.76-6.62 (m, 1H), 6.53 (d, J=15.2 Hz, 1H), 4.76-4.69 (m, 1H), 4.54-4.43 (m, 1H), 4.10-3.76 (m, 4H), 3.72 (d, J=5.4 Hz, 1H), 3.66 (d, J=6.9 Hz, 1H), 2.86 (s, 6H), 2.80-2.70 (m, 1H), 2.46-2.23 (m, 2H), 1.61 (d, J=9.1 Hz, 1H), 1.06 (t, J=7.4 Hz, 3H).MS (ESI) [M+H]⁺=266.2.

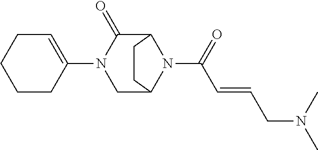 [SMILES: CN(C)C/C=C/C(=O)N1C2CCC1C(=O)N(C1=CCCCC1)C2]

Example 176 (E)-3-(cyclohex-1-en-1-yl)-8-(4-(dimethylamino)but-2-enoyl)-3,8-diazabicyclo[3.2.1]octan-2-one (XS209-30). Example 176 was synthesized following similar procedure for preparing example 28. White solid, 11% yield ¹H NMR (400 MHz, Methanol-d₄) δ 6.95-6.71 (m, 2H), 5.64 (s, 1H), 4.97-4.88 (m, 1H), 4.82-4.64 (m, 1H), 4.00-3.90 (m, 2H), 3.80-3.62 (m, 1H), 3.27-3.06 (m, 1H), 2.90 (s, 6H), 2.42-2.04 (m, 6H), 2.03-1.92 (m, 1H), 1.75-1.64 (m, 2H), 1.64-1.52 (m, 2H), 1.52-1.32 (m, 1H).MS (ESI) [M+H]⁺=318.2.

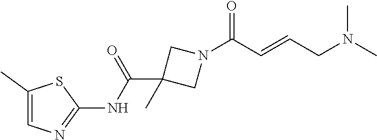

Example 177 (E)-1-(4-(dimethylamino)but-2-enoyl)-3-methyl-N-(5-methylthiazol-2-yl)azetidine-3-carboxamide (XS209-38). Example 177 was synthesized following similar procedure for preparing example 53. White solid, 44% yield ¹H NMR (400 MHz, Methanol-d₄) δ 7.15 (s, 1H), 6.72-6.56 (m, 1H), 6.43 (d, J=15.3 Hz, 1H), 4.65 (d, J=9.1 Hz, 1H), 4.35 (d, J=10.7 Hz, 1H), 4.12 (d, J=9.2 Hz, 1H), 3.87 (d, J=8.2 Hz, 3H), 2.83 (s, 6H), 2.35 (s, 3H), 1.62 (s, 3H).MS (ESI) [M+H]⁻=323.1.

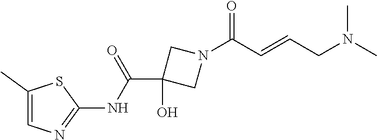

Example 178 (E)-1-(4-(dimethylamino)but-2-enoyl)-3-hydroxy-N-(5-methylthiazol-2-yl)azetidine-3-carboxamide (XS209-39). Example 178 was synthesized following similar procedure for preparing example 53. White solid, 27% yield ¹H NMR (400 MHz, Methanol-d₄) δ 7.13 (s, 1H), 6.81-6.66 (m, 1H), 6.49 (d, J=15.3 Hz, 1H), 4.68 (d, J=9.5 Hz, 1H), 4.43 (d, J=11.0 Hz, 1H), 4.27 (d, J=9.5 Hz, 1H), 4.03 (d, J=11.0 Hz, 1H), 3.91 (d, J=7.1 Hz, 2H), 2.87 (s, 6H), 2.37 (s, 3H).MS (ESI) [M+H]⁺=325.1.

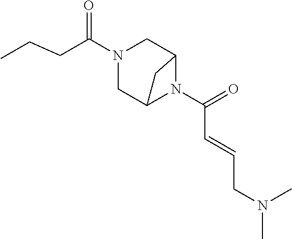 [SMILES: CCCC(=O)N1CC2CC(C1)N2C(=O)/C=C/CN(C)C]

Example 179 (E)-1-(3-butyryl-3,6-diazabicyclo[3.1.1]heptan-6-yl)-4-(dimethylamino)but-2-en-1-one (XS209-40). Example 179 was synthesized following similar procedure for preparing example 79. White solid, 19% yield ¹H NMR (400 MHz, Methanol-d₄) δ 6.84-6.69 (m, 1H), 6.57 (d, J=15.3 Hz, 1H), 4.77 (s, 1H), 4.54 (s, 1H), 4.13 (d, J=11.5 Hz, 1H), 4.02-3.87 (m, 3H), 3.81-3.69 (m, 2H), 2.91 (d, J=4.4 Hz, 6H), 2.82 (q, J=6.6 Hz, 1H), 2.47-2.23 (m, 2H), 1.72-1.58 (m, 3H), 0.98 (t, J=7.3 Hz, 3H).MS (ESI) [M+H]⁺=280.2.

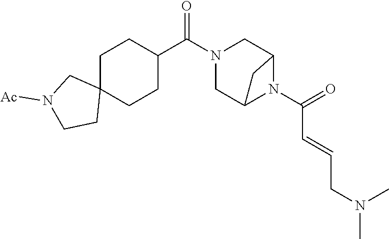 [SMILES: CC(=O)N1CCC2(CCC(C(=O)N3CC4CC(C3)N4C(=O)/C=C/CN(C)C)CC2)C1]

Example 180 (E)-1-(3-(2-acetyl-2-azaspiro[4.5]decane-8-carbonyl)-3,6-diazabicyclo[3.1.1]heptan-6-yl)-4-(dimethylamino)but-2-en-1-one (XS209-53). Example 180 was synthesized following similar procedure for preparing example 79. White solid, 18% yield ¹H NMR (400 MHz, Methanol-d₄) δ 6.67-6.54 (m, 1H), 6.47 (d, J=15.2 Hz, 1H), 4.66 (s, 1H), 4.42 (s, 1H), 4.12-3.91 (m, 1H), 3.87-3.77 (m, 3H), 3.68-3.51 (m, 4H), 3.51-3.40 (m, 1H), 3.15 (d, J=5.7 Hz, 1H), 2.78 (s, 6H), 2.74 (s, 3H), 2.71-2.65 (m, 1H), 2.59-2.46 (m, 1H), 2.13-2.02 (m, 3H), 1.90-1.81 (m, 1H), 1.63-1.50 (m, 5H), 1.46-1.37 (m, 2H).MS (ESI) [M+H]⁺=417.3.

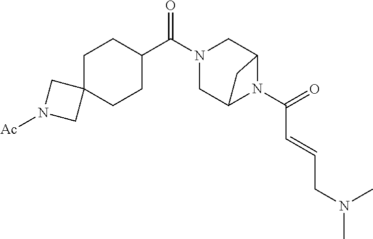 [SMILES: CC(=O)N1CC2(CCC(C(=O)N3CC4CC(C3)N4C(=O)/C=C/CN(C)C)CC2)C1]

Example 181 (E)-1-(3-(2-acetyl-2-azaspiro[3.5]nonane-7-carbonyl)-3,6-diazabicyclo[3.1.1]heptan-6-yl)-4-(dimethylamino)but-2-en-1-one (XS209-54). Example 181 was synthesized following similar procedure for preparing example 79. White solid, 21% yield ¹H NMR (400 MHz, Methanol-d₄) δ 6.71-6.60 (m, 1H), 6.51 (d, J=15.2 Hz, 1H), 4.75-4.64 (m, 1H), 4.49-4.41 (m, 1H), 4.16-3.92 (m, 2H), 3.90-3.81 (m, 4H), 3.70 (d, J=9.3 Hz, 2H), 3.63-3.55 (m, 2H), 2.82 (s, 6H), 2.77-2.69 (m, 1H), 2.54 (t, J=11.2 Hz, 1H), 1.98-1.83 (m, 5H), 1.71 (d, J=13.3 Hz, 1H) 1.65-1.49 (m, 4H), 1.47-1.34 (m, 2H).MS (ESI) [M+H]⁺=403.3.

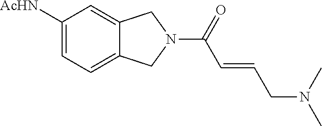 [SMILES: CC(=O)Nc1ccc2c(c1)CN(C(=O)/C=C/CN(C)C)C2]

Example 182 (E)-N-(2-(4-(dimethylamino)but-2-enoyl)isoindolin-5-yl)acetamide (XS209-55). Example 182 was synthesized following similar procedure for preparing example 79. White solid, 39% yield ¹H NMR (400 MHz, Methanol-d4) δ 7.55 (d, J=21.6 Hz, 1H), 7.33-7.21 (m, 1H), 7.15 (t, J=8.1 Hz, 1H), 6.81-6.61 (m, 2H), 4.85 (s, 2H), 4.66 (d, J=7.2 Hz, 2H), 3.89 (d, J=6.5 Hz, 2H), 2.82 (s, 6H), 2.01 (s, 3H).MS (ESI) [M+H]⁺=288.2.

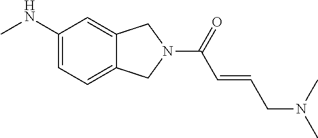

Example 183 (E)-4-(dimethylamino)-1-(5-(methylamino)isoindolin-2-yl)but-2-en-1-one (XS209-57). Example 183 was synthesized following similar procedure for preparing example 79. White solid, 55% yield ¹H NMR (400 MHz, Methanol-d₄) δ 7.49 (t, J=9.0 Hz, 1H), 7.42 (d, J=7.1 Hz, 1H), 7.36 (d, J=8.1 Hz, 1H), 6.84 (d, J=15.2 Hz, 1H), 6.80-6.66 (m, 1H), 5.04 (d, J=10.6 Hz, 2H), 4.83 (d, J=8.7 Hz, 2H), 3.95 (d, J=6.8 Hz, 2H), 3.03 (s, 3H), 2.87 (s, 6H).MS (ESI) [M+H]⁺=260.2.

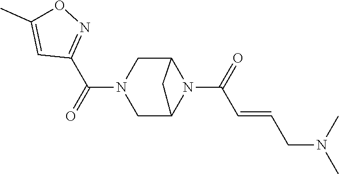

Example 184 (E)-4-(dimethylamino)-1-(5-(methylamino)isoindolin-2-yl)but-2-en-1-one (XS209-60). Example 184 was synthesized following similar procedure for preparing example 79. White solid, 36% yield ¹H NMR (400 MHz, Methanol-d4) δ 6.75-6.62 (m, 1H), 6.60-6.47 (m, 1H), 6.34 (s, 1H), 4.74 (d, J=35.5 Hz, 1H), 4.50 (d, J=33.5 Hz, 1H), 4.29-3.94 (m, 3H), 3.91-3.82 (m, 3H), 2.87-2.78 (m, 7H), 2.42 (d, J=3.3 Hz, 3H), 1.68 (d, J=7.1 Hz, 1H).MS (ESI) [M+H]⁺=319.2.

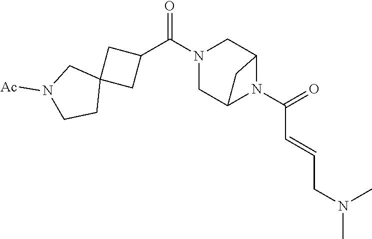 [SMILES: CC(=O)N1CCC2(CC(C(=O)N3CC4CC(C3)N4C(=O)/C=C/CN(C)C)C2)C1]

Example 185 (E)-1-(3-(6-acetyl-6-azaspiro[3.4]octane-2-carbonyl)-3,6-diazabicyclo[3.1.1]heptan-6-yl)-4-(dimethylamino)but-2-en-1-one (XS209-62). Example 185 was synthesized following similar procedure for preparing example 79. White solid, 19% yield ¹H NMR (400 MHz, Methanol-d₄) δ 6.72-6.58 (m, 1H), 6.47 (d, J=15.2 Hz, 1H), 4.77-4.60 (m, 1H), 4.58-4.38 (m, 1H), 4.03-3.67 (m, 5H), 3.53-3.24 (m, 5H), 2.80 (s, 6H), 2.75-2.65 (m, 1H), 2.27-2.05 (m, 4H), 2.00-1.90 (m, 3H), 1.89-1.82 (m, 1H), 1.80-1.74 (m, 1H), 1.55 (d, J=8.8 Hz, 1H), 1.46-1.40 (m, 1H).MS (ESI) [M+H]⁺=389.2.

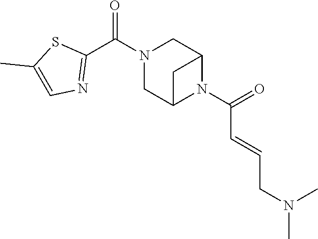

Example 186 (E)-4-(dimethylamino)-1-(3-(5-methylthiazole-2-carbonyl)-3,6-diazabicyclo[3.1.1]heptan-6-yl)but-2-en-1-one (XS209-65). Example 186 was synthesized following similar procedure for preparing example 79. White solid, 44% yield ¹H NMR (400 MHz, Methanol-d₄) δ 7.46 (d, J=9.5 Hz, 1H), 6.65-6.50 (m, 1H), 6.49-6.34 (m, 1H), 4.65 (d, J=14.6 Hz, 1H), 4.50-4.24 (m, 3H), 3.99-3.71 (m, 4H), 2.75-2.60 (m, 7H), 2.35 (s, 3H), 1.57 (d, J=9.2 Hz, 1H).MS (ESI) [M+H]⁺=335.1.

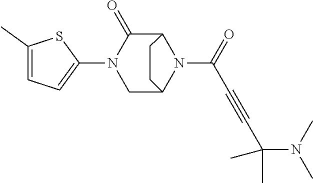

Example 187 8-(4-(dimethylamino)-4-methylpent-2-ynoyl)-3-(5-methylthiophen-2-yl)-3,8-diazabicyclo[3.2.1]octan-2-one (XS209-74). Example 187 was synthesized following similar procedure for preparing example 28. White solid, 29% yield ¹H NMR (400 MHz, Methanol-d₄) δ 6.49 (s, 2H), 4.99 (t, J=5.5 Hz, 1H), 4.78 (d, J=6.5 Hz, 1H), 4.04-3.90 (m, 1H), 3.73-3.54 (m, 1H), 2.97-2.88 (m, 6H), 2.40-2.20 (m, 5H), 2.18-2.08 (m, 1H), 2.04-1.89 (m, 1H), 1.77-1.64 (m, 6H).MS (ESI) [M+H]⁻=360.2.

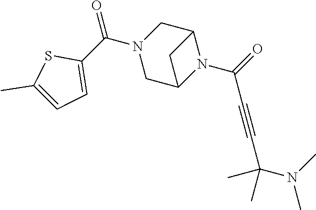

Example 188 4-(dimethylamino)-4-methyl-1-(3-(5-methylthiophene-2-carbonyl)-3,6-diazabicyclo[3.1.1]heptan-6-yl)pent-2-yn-1-one (XS209-75). Example 188 was synthesized following similar procedure for preparing example 79. White solid, 34% yield ¹H NMR (400 MHz, Methanol-d₄) δ 7.43-7.28 (m, 1H), 6.75 (t, J=4.5 Hz, 1H), 4.71-4.47 (m, 2H), 4.40-4.20 (m, 1H), 4.20-4.06 (m, 1H), 4.06-3.78 (m, 2H), 3.13-3.00 (m, 1H), 2.92-2.85 (m, 8H), 2.74 (q, J=7.1 Hz, 1H), 2.41 (s, 3H), 1.73-1.55 (m, 4H).MS (ESI) [M+H]⁺=360.1.

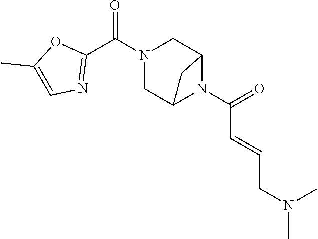

Example 189 (E)-4-(dimethylamino)-1-(3-(5-methyloxazole-2-carbonyl)-3,6-diazabicyclo[3.1.1]heptan-6-yl)but-2-en-1-one (XS209-92). Example 189 was synthesized following similar procedure for preparing example 79. White solid, 29% yield ¹H NMR (400 MHz, Methanol-d₄) δ 6.89 (s, 1H), 6.66-6.54 (m, 1H), 6.52-6.38 (m, 1H), 4.73-4.63 (m, 1H), 4.48-4.22 (m, 3H), 3.94-3.72 (m, 4H), 2.77-2.66 (m, 7H), 2.26 (s, 3H), 1.59 (d, J=9.2 Hz, 1H).MS (ESI) [M+H]⁻=319.2.

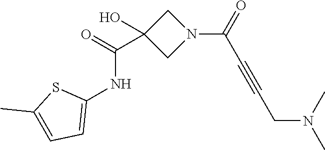

Example 190 1-(4-(dimethylamino)but-2-ynoyl)-3-hydroxy-N-(5-methylthiophen-2-yl)azetidine-3-carboxamide (XS209-99). Example 190 was synthesized following similar procedure for preparing example 53. White solid, 29% yield ¹H NMR (400 MHz, Methanol-d₄) δ 6.55 (d, J=3.4 Hz, 1H), 6.41 (d, J=4.2 Hz, 1H), 4.55 (d, J=9.7 Hz, 1H), 4.31 (d, J=11.0 Hz, 1H), 4.23 (s, 2H), 4.13 (d, J=9.7 Hz, 1H), 3.90 (d, J=10.9 Hz, 1H), 2.88 (s, 6H), 2.28 (s, 3H). MS (ESI) [M+H]⁺=322.1.

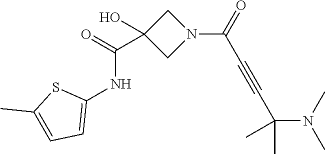

Example 191 1-(4-(dimethylamino)-4-methylpent-2-ynoyl)-3-hydroxy-N-(5-methylthiophen-2-yl)azetidine-3-carboxamide (XS209-100). Example 191 was synthesized following similar procedure for preparing example 53. White solid, 38% yield ¹H NMR (400 MHz, Methanol-d₄) δ 6.65-6.20 (m, 2H), 4.62-4.43 (m, 1H), 4.34 (d, 0.1=11.0 Hz, 1H), 4.20-4.02 (m, 1H), 3.97-3.62 (m, 1H), 2.92 (s, 6H), 2.31 (s, 3H), 1.69 (s, 6H). MS (ESI) [M+H]⁺=350.2.

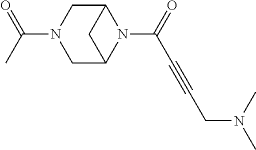 [SMILES: CC(=O)N1CC2CC(C1)N2C(=O)C#CCN(C)C]

Example 192 1-(3-acetyl-3,6-diazabicyclo[3.1.1]heptan-6-yl)-4-(dimethylamino)but-2-yn-1-one (XS209-101). Example 192 was synthesized following similar procedure for preparing example 79. White solid, 21% yield ¹H NMR (400 MHz, Methanol-d₄) δ 4.65-4.55 (m, 1H), 4.54-4.43 (m, 1H), 4.34-4.18 (m, 2H), 4.05-3.91 (m, 1H), 3.85-3.75 (m, 1H), 3.71-3.59 (m, 2H), 2.92 (d, J=4.1 Hz, 7H), 2.80-2.68 (m, 1H), 2.07-1.98 (m, 3H), 1.69-1.55 (m, 1H). MS (ESI) [M+H]⁺=250.2.

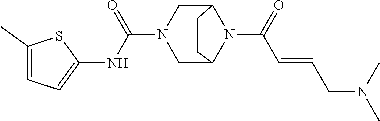

Example 193 (E)-8-(4-(dimethylamino)but-2-enoyl)-N-(5-methylthiophen-2-yl)-8-azabicyclo[3.2.1]octane-3-carboxamide (XS209-122). Example 193 was synthesized following similar procedure for preparing example 53. White solid ¹H NMR (400 MHz, Methanol-d₄) δ 6.79 (d, J=15.2 Hz, 1H), 6.72-6.60 (m, 1H), 6.38 (s, 2H), 4.62 (d, J=6.6 Hz, 1H), 4.52-4.40 (m, 1H), 3.86 (d, J=7.0 Hz, 2H), 3.03-2.90 (m, 1H), 2.82 (s, 6H), 2.26 (s, 3H), 2.13-1.99 (m, 1H), 1.98-1.59 (m, 7H). MS (ESI) [M+H]⁺=362.2.

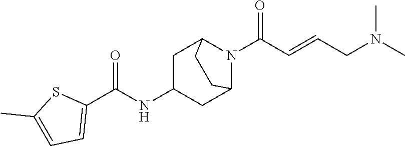

Example 194 (E)-N-(8-(4-(dimethylamino)but-2-enoyl)-8-azabicyclo[3.2.1]octan-3-yl)-5-methylthiophene-2-carboxamide (XS209-139). Example 194 was synthesized following similar procedure for preparing example 79. White solid, 45% yield ¹H NMR (400 MHz, Methanol-d4) δ 7.34 (d, J=3.2 Hz, 1H), 6.80-6.51 (m, 3H), 4.51 (s, 1H), 4.37 (s, 1H), 3.89 (s, 1H), 3.81 (d, J=6.8 Hz, 2H), 2.76 (s, 6H), 2.35 (s, 3H), 2.15-2.05 (m, 1H), 2.05-1.91 (m, 5H), 1.90-1.76 (m, 2H). MS (ESI) [M+H]⁺=362.2.

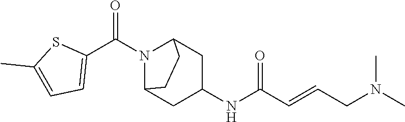

Example 195 (E)-4-(dimethylamino)-N-(8-(5-methylthiophene-2-carbonyl)-8-azabicyclo[3.2.1]octan-3-yl)but-2-enamide (XS209-140). Example 195 was synthesized following similar procedure for preparing example 79. White solid, 36% yield ¹H NMR (400 MHz, Methanol-d4) δ 7.20 (d, J=3.4 Hz, 1H), 6.72-6.63 (m, 1H), 6.62-6.49 (m, 1H), 6.35 (d, J=15.3 Hz, 1H), 4.56 (s, 2H), 3.95 (t, J=5.4 Hz, 1H), 3.79 (d, J=7.1 Hz, 2H), 2.76 (s, 6H), 2.37 (s, 3H) 2.26-2.10 (m, 2H), 2.01-1.85 (m, 4), 1.80 (d, J 14.4 Hz, 2H). MS (ESI) [M+H]⁺=362.2.

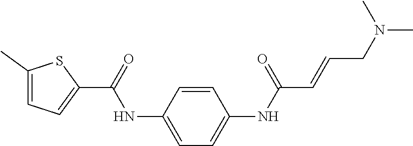

Example 196 (E)-N-(4-(4-(dimethylamino)but-2-enamido)phenyl)-5-methylthiophene-2-carboxamide (XS209-165). Example 196 was synthesized following similar procedure for preparing example 79. White solid, 33% yield ¹H NMR (400 MHz, Methanol-d₄) δ 7.71 (d, J=3.5 Hz, 1H), 7.65 (s, 4H), 6.93-6.76 (m, 2H), 6.56 (d, J=15.2 Hz, 1H), 4.00 (d, J=7.2 Hz, 2H), 2.94 (s, 6H), 2.55 (s, 3H). MS (ESI) [M+H]⁺=344.1.

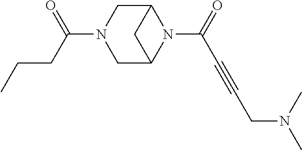 [SMILES: CCCC(=O)N1CC2CC(C1)N2C(=O)C#CCN(C)C]

Example 197 1-(3-butyryl-3,6-diazabicyclo[3.1.1]heptan-6-yl)-4-(dimethylamino)but-2-yn-1-one (XS209-166). Example 197 was synthesized following similar procedure for preparing example 79. White solid, 18% yield ¹H NMR (400 MHz, Methanol-d₄) δ 4.62 (s, 1H), 4.59-4.47 (m, 1H), 4.27 (s, 2H), 4.04-3.93 (m, 1H), 3.87-3.77 (m, 1H), 3.77-3.62 (m, 2H), 2.94-2.90 (m, 6H), 2.79-2.64 (m, 1H), 2.40-2.20 (m, 2H), 1.64-1.52 (m, 3H), 0.97-0.88 (m, 3H).MS (ESI) [M+H]⁺=278.2.

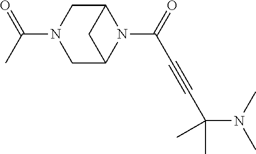 [SMILES: CC(=O)N1CC2CC(C1)N2C(=O)C#CC(C)(C)N(C)C]

Example 198 1-(3-acetyl-3,6-diazabicyclo[3.1.1]heptan-6-yl)-4-(dimethylamino)-4-methylpent-2-yn-1-one (XS209-167). Example 198 was synthesized following similar procedure for preparing example 79. White solid, 19% yield ¹H NMR (400 MHz, Methanol-d₄) δ 4.66 (d, J=6.2 Hz, 2H), 4.10 (d, J=12.0 Hz, 1H), 3.94 (d, J=13.6 Hz, 1H), 3.86 (d, J=12.1 Hz, 1H), 3.75 (d, J=13.6 Hz, 1H), 3.15-3.04 (m, 1H), 2.93 (s, 6H), 2.92 (s, 6H), 2.12 (s, 3H), 1.81 (d, J=9.0 Hz, 1H). MS (ESI) [M+H]⁺=278.2.

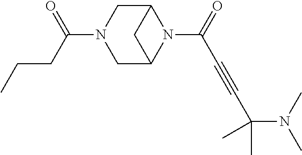 [SMILES: CCCC(=O)N1CC2CC(C1)N2C(=O)C#CC(C)(C)N(C)C]

Example 199 1-(3-butyryl-3,6-diazabicyclo[3.1.1]heptan-6-yl)-4-(dimethylamino)-4-methylpent-2-yn-1-one (XS209-168). Example 199 was synthesized following similar procedure for preparing example 79. White solid, 20% yield ¹H NMR (400 MHz, Methanol-d₄) δ 4.64 (d, J=5.8 Hz, 2H), 4.06 (d, J=11.9 Hz, 1H), 3.91 (d, J=13.5 Hz, 1H), 3.84 (d, J=12.1 Hz, 1H), 3.72 (d, J=13.4 Hz, 1H), 3.07 (q, J=7.4 Hz, 1H), 2.92-2.86 (m, 12H), 2.40-2.29 (m, 2H), 1.76 (d, J=7.9 Hz, 1H), 1.63-1.56 (m, 2H), 0.92 (t, J=7.3 Hz, 3H). MS (ESI) [M+H]⁺=306.2.

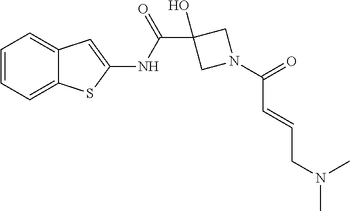 [SMILES: CN(C)C/C=C/C(=O)N1CC(O)(C(=O)Nc2cc3ccccc3s2)C1]

Example 200: 1-(3-butyryl-3,6-diazabicyclo[3.1.1]heptan-6-yl)-4-(dimethylamino)-4-methylpent-2-yn-1-one (XS209-174). Example 200 was synthesized following similar procedure for preparing example 53. White solid, 45% yield ¹H NMR (400 MHz, Methanol-d₄) δ 7.66 (d, J=7.8 Hz, 1H), 7.55 (d, J=7.8 Hz, 1H), 7.21 (t, J=7.5 Hz, 1H), 7.14 (t, J=7.4 Hz, 1H), 7.06 (s, 1H), 6.74-6.61 (m, 1H), 6.47 (d, J=15.2 Hz, 1H), 4.66 (d, J=9.5 Hz, 1H), 4.40 (d, J=11.0 Hz, 1H), 4.23 (d, J=9.5 Hz, 1H), 3.98 (d, J=11.0 Hz, 1H), 3.87 (d, J=7.1 Hz, 2H), 2.82 (s, 6H). MS (ESI) [M+H]⁺=360.1.

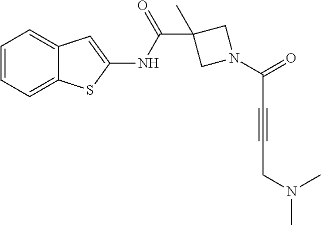 [SMILES: CN(C)CC#CC(=O)N1CC(C)(C(=O)Nc2cc3ccccc3s2)C1]

Example 201: N-(benzo[b]thiophen-2-yl)-1-(4-(dimethylamino)but-2-ynoyl)-3-methylazetidine-3-carboxamide (XS209-175). Example 201 was synthesized following similar procedure for preparing example 53. White solid, 44% yield ¹H NMR (400 MHz, Methanol-d₄) δ 7.67 (d, J=7.9 Hz, 1H), 7.55 (d, J=7.8 Hz, 1H), 7.22 (t, J=7.4 Hz, 1H), 7.15 (t, J=7.4 Hz, 1H), 6.94 (s, 1H), 4.61 (d, J=9.2 Hz, 1H), 4.32 (d, J=10.6 Hz, 1H), 4.27 (s, 2H), 4.05 (d, J=9.2 Hz, 1H), 3.87 (d, J=10.6 Hz, 1H), 2.93 (s, 6H), 1.63 (s, 3H).MS (ESI) [M+H]⁻=345.2.

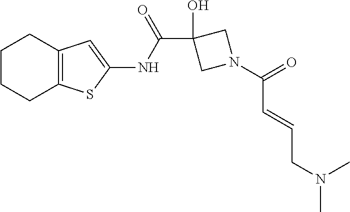 [SMILES: CN(C)C/C=C/C(=O)N1CC(O)(C(=O)Nc2cc3c(s2)CCCC3)C1]

Example 202: (E)-1-(4-(dimethylamino)but-2-enoyl)-3-hydroxy-N-(4,5,6,7-tetrahydrobenzo[b]thiophen-2-yl)azetidine-3-carboxamide (XS209-176). Example 202 was synthesized following similar procedure for preparing example 53. White solid, 39% yield ¹H NMR (400 MHz, Methanol-d₄) δ 6.70-6.55 (m, 1H), 6.49-6.36 (m, 2H), 4.58 (d, J=9.4 Hz, 1H), 4.32 (d, J=10.9 Hz, 1H), 4.16 (d, J=9.4 Hz, 1H), 3.91 (d, J=10.9 Hz, 1H), 3.84 (d, J=7.1 Hz, 2H), 2.79 (s, 6H), 2.54 (t, J=6.2 Hz, 2H), 2.40 (t, J=5.9 Hz, 2H), 1.79-1.59 (m, 4H). MS (ESI) [M+H]⁺=364.2.

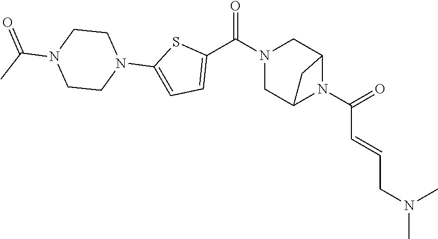 [SMILES: CC(=O)N1CCN(c2ccc(C(=O)N3CC4CC(C3)N4C(=O)/C=C/CN(C)C)s2)CC1]

Example 203: (E)-1-(3-(5-(4-acetylpiperazin-1-yl)thiophene-2-carbonyl)-3,6-diazabicyclo[3.1.1]heptan-6-yl)-4-(dimethylamino)but-2-en-1-one (XS209-184). Example 203 was synthesized following similar procedure for preparing example 79. White solid, 28% yield ¹H NMR (400 MHz, Methanol-d₄) δ 7.46 (s, 1H), 6.84-6.69 (m, 1H), 6.60 (d, J=15.3 Hz, 1H), 6.25 (d, J=4.1 Hz, 1H), 4.82 (s, 1H), 4.60 (s, 1H), 4.02-3.87 (m, 3H), 3.79-3.63 (m, 4H), 3.38-3.30 (m, 7H), 2.91 (s, 6H), 2.87-2.79 (m, 1H), 2.17 (s, 3H), 1.74 (d, J=9.1 Hz, 1H). MS (ESI) [M+H]⁺=446.2

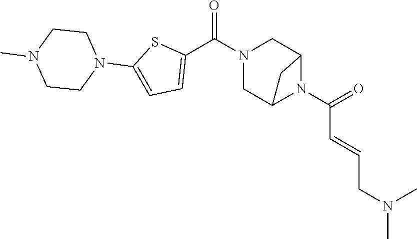

Example 204: (E)-4-(dimethylamino)-1-(3-(5-(4-methylpiperazin-1-yl)thiophene-2-carbonyl)-3,6-diazabicyclo[3.1.1]heptan-6-yl)but-2-en-1-one (XS209-185). Example 204 was synthesized following similar procedure for preparing example 79. White solid, 23% yield ¹H NMR (400 MHz, Methanol-d₄) δ 7.32 (s, 1H), 6.70-6.58 (m, 1H), 6.47 (d, J=15.3 Hz, 1H), 6.20 (d, J=4.1 Hz, 1H), 4.69 (s, 1H), 4.46 (s, 1H), 4.37-3.89 (m, 3H), 3.83 (d, J=7.0 Hz, 3H), 3.73 (d, J=9.8 Hz, 2H), 3.57-3.39 (m, 2H), 3.19-3.07 (m, 4H), 2.85 (s, 3H), 2.77 (s, 6H), 2.74-2.65 (m, 1H), 1.60 (d, J=9.1 Hz, 1H).MS (ESI) [M+H]⁺=418.2

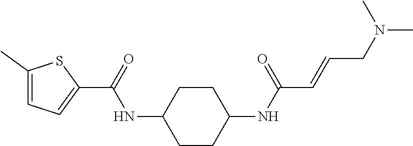

Example 205: (E)-N-(4-(4-(dimethylamino)but-2-enamido)cyclohexyl)-5-methylthiophene-2-carboxamide (XS224-6). Example 205 was synthesized following similar procedure for preparing example 79. White solid, 25% yield ¹H NMR (400 MHz, Methanol-d₄) δ 7.53-7.31 (m, 1H), 6.73 (s, 1H), 6.70-6.53 (m, 1H), 6.29 (d, J=15.3 Hz, 1H), 3.87 (d, J=6.7 Hz, 2H), 3.84-3.62 (m, 2H), 2.84 (s, 6H), 2.44 (s, 3H), 1.94 (d, J=9.3 Hz, 4H), 1.57-1.22 (m, 4H).MS (ESI) [M+H]⁺=350.2.

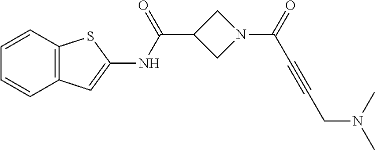 [SMILES: CN(C)CC#CC(=O)N1CC(C(=O)Nc2cc3ccccc3s2)C1]

Example 206: N-(benzo[b]thiophen-2-yl)-1-(4-(dimethylamino)but-2-ynoyl)azetidine-3-carboxamide (XS224-106). Example 206 was synthesized following similar procedure for preparing example 53. White solid, 44% yield ¹H NMR (400 MHz, Methanol-d₄) δ 7.72 (d, J=7.8 Hz, 1H), 7.61 (d, J=7.8 Hz, 1H), 7.28 (t, J=7.5 Hz, 1H), 7.21 (t, J=7.5 Hz, 1H), 6.95 (s, 1H), 4.49 (p, J=9.3 Hz, 2H), 4.31 (d, J=13.3 Hz, 3H), 4.25-4.17 (m, 1H), 3.67 (p, J=7.1, 6.0 Hz, 1H), 2.98 (s, 6H). MS (ESI) [M+H]⁺=342.1.

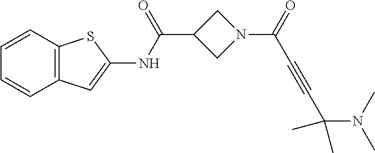 [SMILES: CN(C)C(C)(C)C#CC(=O)N1CC(C(=O)Nc2cc3ccccc3s2)C1]

Example 207: N-(benzo[b]thiophen-2-yl)-1-(4-(dimethylamino)-4-methylpent-2-ynoyl)azetidine-3-carboxamide (XS224-107). Example 207 was synthesized following similar procedure for preparing example 53. White solid, 39% yield ¹H NMR (400 MHz, Methanol-d₄) δ 7.72 (d, J=7.8 Hz, 1H), 7.60 (d, J=7.8 Hz, 1H), 7.28 (t, J=7.5 Hz, 1H), 7.20 (t, J=7.4 Hz, 1H), 6.95 (s, 1H), 4.48 (p, J=8.7 Hz, 2H), 4.29 (t, J=9.6 Hz, 1H), 4.24-4.16 (m, 1H), 3.67 (p, J=7.2, 6.0 Hz, 1H), 2.98 (s, 6H), 1.75 (s, 6H). MS (ESI) [M+H]⁺=370.2.

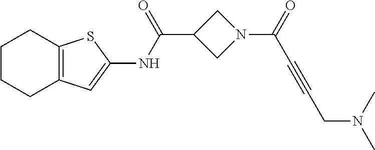 [SMILES: CN(C)CC#CC(=O)N1CC(C(=O)Nc2cc3c(s2)CCCC3)C1]

Example 208: 1-(4-(dimethylamino)but-2-ynoyl)-N-(4,5,6,7-tetrahydrobenzo[b]thiophen-2-yl)azetidine-3-carboxamide (XS224-108). Example 208 was synthesized following similar procedure for preparing example 53. White solid, 38% yield ¹H NMR (400 MHz, Methanol-d₄) δ 6.33 (s, 1H), 4.48-4.35 (m, 2H), 4.29 (s, 2H), 4.25-4.17 (m, 1H), 4.14-4.08 (m, 1H), 3.62-3.51 (m, 1H), 2.95 (s, 6H), 2.60 (t, J=6.3 Hz, 2H), 2.45 (t, J=5.7 Hz, 2H), 1.85-1.67 (m, 4H). MS (ESI) [M+H]⁺=346.2.

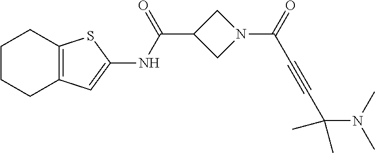 [SMILES: CN(C)C(C)(C)C#CC(=O)N1CC(C(=O)Nc2cc3c(s2)CCCC3)C1]

Example 209: 1-(4-(dimethylamino)-4-methylpent-2-ynoyl)-N-(4,5,6,7-tetrahydrobenzo[b]thiophen-2-yl)azetidine-3-carboxamide (XS224-109). Example 209 was synthesized following similar procedure for preparing example 53. White solid, 32% yield ¹H NMR (400 MHz, Methanol-d₄) δ 6.34 (s, 1H), 4.41 (p, J=8.9 Hz, 2H), 4.22 (t, J=9.7 Hz, 1H), 4.16-4.07 (m, 1H), 3.63-3.49 (m, 1H), 2.96 (s, 6H), 2.60 (t, J=6.2 Hz, 2H), 2.46 (t, J=5.7 Hz, 2H), 1.84-1.67 (m, 10H).MS (ESI) [M+H]⁺=374.2.

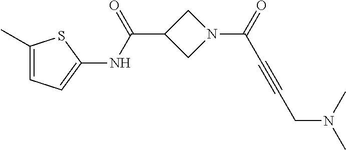

Example 210: 1-(4-(dimethylamino)but-2-ynoyl)-N-(5-methylthiophen-2-yl)azetidine-3-carboxamide (XS224-110). Example 210 was synthesized following similar procedure for preparing example 53. White solid, 29% yield ¹H NMR (400 MHz, Methanol-d₄) δ 6.47 (s, 2H), 4.42 (p, J=9.1 Hz, 2H), 4.31 (s, 2H), 4.23 (t, J=9.7 Hz, 1H), 4.17-4.09 (m, 1H), 3.64-3.50 (m, 1H), 2.96 (s, 6H), 2.35 (s, 3H).MS (ESI) [M+H]⁺=306.1.

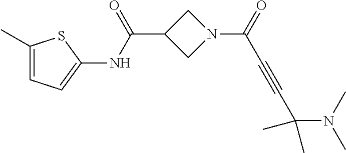

Example 211: 1-(4-(dimethylamino)-4-methylpent-2-ynoyl)-N-(5-methylthiophen-2-yl)azetidine-3-carboxamide (XS224-111). Example 211 was synthesized following similar procedure for preparing example 53. White solid, 29% yield ¹H NMR (400 MHz, Methanol-d₄) δ 6.48 (s, 2H), 4.49-4.34 (m, 2H), 4.24 (t, J=9.6 Hz, 1H), 4.15 (d, J=5.6 Hz, 1H), 3.64-3.54 (m, 1H), 2.97 (s, 6H), 2.36 (s, 3H), 1.74 (s, 6H). MS (ESI) [M+H]⁺=334.2.

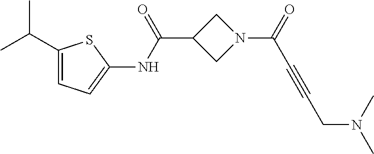 [SMILES: CC(C)c1ccc(NC(=O)C2CN(C(=O)C#CCN(C)C)C2)s1]

Example 212: 1-(4-(dimethylamino)but-2-ynoyl)-N-(5-isopropylthiophen-2-yl)azetidine-3-carboxamide (XS224-116). Example 212 was synthesized following similar procedure for preparing example 53. White solid, 35% yield ¹H NMR (400 MHz, Methanol-d₄) δ 6.51 (d, J=5.6 Hz, 2H), 4.43 (p, J=8.9 Hz, 2H), 4.31 (s, 2H), 4.24 (t, J=9.6 Hz, 1H), 4.14 (d, J=15.8 Hz, 1H), 3.58 (p, J=7.0, 6.2 Hz, 1H), 3.09-3.00 (m, 1H), 2.96 (s, 6H), 1.27 (d, J=6.8 Hz, 6H). MS (ESI) [M+H]⁺=334.2.

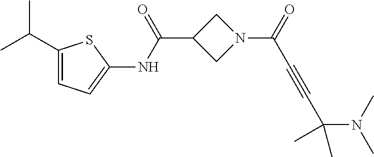 [SMILES: CC(C)c1ccc(NC(=O)C2CN(C(=O)C#CC(C)(C)N(C)C)C2)s1]

Example 213: 1-(4-(dimethylamino)-4-methylpent-2-ynoyl)-N-(5-isopropylthiophen-2-yl)azetidine-3-carboxamide (XS224-117). Example 213 was synthesized following similar procedure for preparing example 53. White solid, 25% yield ¹H NMR (400 MHz, Methanol-d₄) δ 6.51 (d, J=5.9 Hz, 2H), 4.50-4.34 (m, 2H), 4.24 (t, J=9.6 Hz, 1H), 4.18-4.11 (m, 1H), 3.58 (p, J=7.2, 6.0 Hz, 1H), 3.11-3.00 (m, 1H), 2.97 (s, 6H), 1.74 (s, 6H), 1.27 (d, J=6.8 Hz, 6H). MS (ESI) [M+H]⁺=362.1.

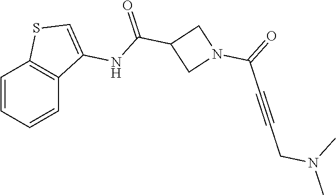 [SMILES: CN(C)CC#CC(=O)N1CC(C(=O)Nc2csc3ccccc23)C1]

Example 214: N-(benzo[b]thiophen-3-yl)-1-(4-(dimethylamino)but-2-ynoyl)azetidine-3-carboxamide (XS224-118). Example 214 was synthesized following similar procedure for preparing example 53. White solid, 44% yield ¹H NMR (400 MHz, Methanol-d₄) δ 7.97-7.88 (m, 2H), 7.83 (d, J=7.2 Hz, 1H), 7.37 (p, J=6.9 Hz, 2H), 4.50 (d, J=6.6 Hz, 2H), 4.29 (d, J=14.2 Hz, 3H), 4.25-4.16 (m, 1H), 3.82 (p, J=7.0, 6.5 Hz, 1H), 2.97 (s, 6H). MS (ESI) [M+H]⁺=342.1.

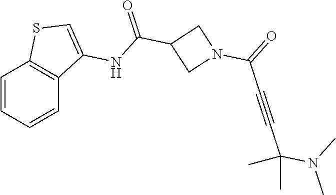 [SMILES: CN(C)C(C)(C)C#CC(=O)N1CC(C(=O)Nc2csc3ccccc23)C1]

Example 215: N-(benzo[b]thiophen-3-yl)-1-(4-(dimethylamino)-4-methylpent-2-ynoyl)azetidine-3-carboxamide (XS224-119). Example 215was synthesized following similar procedure for preparing example 53. White solid, 37% yield ¹H NMR (400 MHz, Methanol-d₄) δ 7.99-7.87 (m, 2H), 7.83 (d, J=7.9 Hz, 1H), 7.37 (p, J=7.0 Hz, 2H), 4.49 (d, J=6.3 Hz, 2H), 4.30 (t, J=9.6 Hz, 1H), 4.25-4.16 (m, 1H), 3.82 (p, J=7.0, 6.1 Hz, 1H), 2.97 (s, 6H), 1.74 (s, 6H). MS (ESI) [M+H]⁺=370.2.

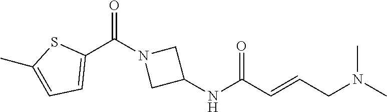

Example 216:(E)-4-(dimethylamino)-N-(1-(5-methylthiophene-2-carbonyl)azetidin-3-yl)but-2-enamide (XS224-143). Example 216 was synthesized following similar procedure for preparing example 79. White solid, 33% yield ¹H NMR (400 MHz, Methanol-d₄) δ 7.29 (d, J=3.1 Hz, 1H), 6.78 (s, 1H), 6.74-6.59 (m, 1H), 6.31 (d, J=15.3 Hz, 1H), 4.82-4.61 (m, 2H), 4.35 (d, J=38.0 Hz, 2H), 3.98 (s, 1H), 3.88 (d, J=7.2 Hz, 2H), 2.83 (s, 6H), 2.45 (s, 3H). MS (ESI) [M+H]⁺=308.2.

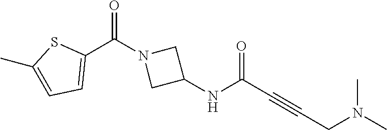

Example 217: 4-(dimethylamino)-N-(1-(5-methylthiophene-2-carbonyl)azetidin-3-yl)but-2-ynamide (XS224-144). Example 217 was synthesized following similar procedure for preparing example 79. White solid, 29% yield ¹H NMR (400 MHz, Methanol-d₄) δ 7.29 (d, J=3.2 Hz, 1H), 6.79 (s, 1H), 4.81-4.60 (m, 2H), 4.47-4.20 (m, 4H), 3.99 (s, 1H), 2.94 (s, 6H), 2.45 (s, 3H). MS (ESI) [M+H]⁺=306.2.

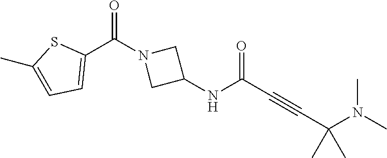

Example 218: 4-(dimethylamino)-4-methyl-N-(1-(5-methylthiophene-2-carbonyl)azetidin-3-yl)pent-2-ynamide (XS224-145). Example 218 was synthesized following similar procedure for preparing example 79. White solid, 29% yield ¹H NMR (400 MHz, Methanol-d₄) δ 7.28 (d, J=3.0 Hz, 1H), 6.78 (s, 1H), 4.81-4.59 (m, 2H), 4.45-4.23 (m, 2H), 3.97 (s, 1H), 2.93 (s, 6H), 2.44 (s, 3H), 1.68 (s, 6H). MS (ESI) [M+H]⁺=334.2.

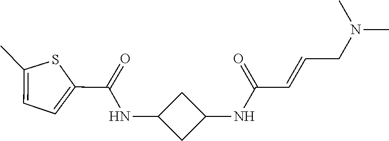

Example 219: (E)-N-(3-(4-(dimethylamino)but-2-enamido)cyclobutyl)-5-methylthiophene-2-carboxamide (XS224-147). Example 219 was synthesized following similar procedure for preparing example 79. White solid, 45% yield ¹H NMR (400 MHz, Methanol-d₄) δ 7.48-7.35 (m, 1H), 6.71 (s, 1H), 6.69-6.54 (m, 1H), 6.34-6.20 (m, 1H), 4.46 (p, J=6.9, 6.4 Hz, 1H), 4.38-4.26 (m, 1H), 3.89-3.77 (m, 2H), 2.80 (s, 6H), 2.73-2.61 (m, 1H), 2.47-2.35 (m, 4H), 2.35-2.25 (m, 1H), 2.08-1.91 (m, 1H). MS (ESI) [M+H]⁺=322.1.

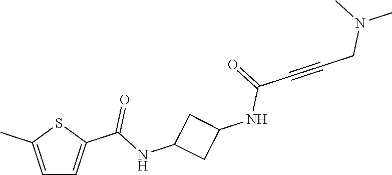

Example 220: N-(3-(4-(dimethylamino)but-2-ynamido)cyclobutyl)-5-methylthiophene-2-carboxamide (XS224-148). Example 220 was synthesized following similar procedure for preparing example 79. White solid, 34% yield ¹H NMR (400 MHz, Methanol-d₄) δ 7.48 (d, J=3.0 Hz, 1H), 6.75 (s, 1H), 4.56-4.43 (m, 1H), 4.35 (dd, J=21.3, 14.8 Hz, 1H), 4.25 (d, J=6.2 Hz, 2H), 2.96 (s, 6H), 2.50-2.40 (m, 5H), 2.40-2.29 (m, 2H). MS (ESI) [M+H]⁺=320.4.

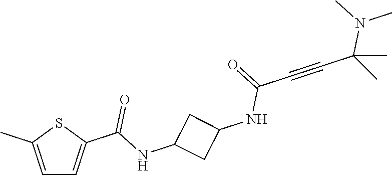

Example 221: N-(3-(4-(dimethylamino)-4-methylpent-2-ynamido)cyclobutyl)-5-methylthiophene-2-carboxamide (XS224-149). Example 221 was synthesized following similar procedure for preparing example 79. White solid, 29% yield ¹H NMR (400 MHz, Methanol-d₄) δ 7.46 (d, J=13.8 Hz, 1H), 6.75 (s, 1H), 4.56-3.99 (m, 2H), 3.00-2.89 (m, 9H), 2.62-2.52 (m, 1H), 2.51-2.39 (m, 5H), 2.39-2.29 (m, 1H), 1.71 (s, 3H). MS (ESI) [M+H]⁺=348.3.

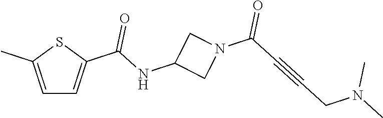

Example 222: N-(1-(4-(dimethylamino)but-2-ynoyl)azetidin-3-yl)-5-methylthiophene-2-carboxamide (XS224-150). Example 222 was synthesized following similar procedure for preparing example 79. White solid, 34% yield ¹H NMR (400 MHz, Methanol-d₄) δ 7.51 (s, 1H), 6.80 (s, 1H), 4.80-4.68 (m, 1H), 4.59 (t, J=8.7 Hz, 1H), 4.40-4.32 (m, 1H), 4.31 (s, 2H), 4.29-4.23 (m, 1H), 4.13-4.04 (m, 1H), 2.97 (s, 6H), 2.48 (s, 3H). MS (ESI) [M+H]⁺=306.2.

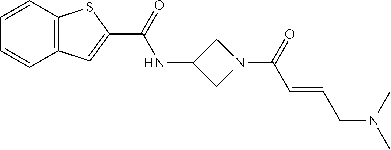 [SMILES: CN(C)C/C=C/C(=O)N1CC(NC(=O)c2cc3ccccc3s2)C1]

Example 223: (E)-N-(1-(4-(dimethylamino)but-2-enoyl)azetidin-3-yl)benzo[b]thiophene-2-carboxamide (XS224-154). Example 223 was synthesized following similar procedure for preparing example 79. White solid, 36% yield ¹H NMR (400 MHz, Methanol-d₄) δ 7.98 (s, 1H), 7.88 (t, J=6.9 Hz, 2H), 7.42 (p, J=6.9 Hz, 2H), 6.77-6.62 (m, 1H), 6.50 (d, J=15.3 Hz, 1H), 4.83-4.76 (m, 1H), 4.67 (t, J=8.5 Hz, 1H), 4.42 (t, J=9.5 Hz, 1H), 4.37-4.29 (m, 1H), 4.19-4.11 (m, 1H), 3.93 (d, J=7.0 Hz, 2H), 2.88 (s, 6H). MS (ESI) [M+H]⁺=344.2.

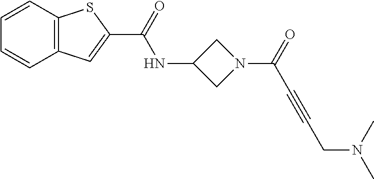 [SMILES: CN(C)CC#CC(=O)N1CC(NC(=O)c2cc3ccccc3s2)C1]

Example 224: A-(1-(4-(dimethylamino) but-2-ynoyl)azetidin-3-yl)benzo[b]thiophene-2-carboxamide (XS224-155). Example 224 was synthesized following similar procedure for preparing example 79. White solid, 29% yield ¹H NMR (400 MHz, Methanol-d₄) δ 7.99 (s, 1H), 7.90 (t, J=6.4 Hz, 2H), 7.43 (p, J=7.0 Hz, 2H), 4.84-4.78 (m, 1H), 4.66 (t, J=8.7 Hz, 1H), 4.42 (t, J=9.5 Hz, 1H), 4.37-4.29 (m, 3H), 4.20-4.08 (m, 1H), 2.99 (s, 6H). MS (ESI) [M+H]⁺=342.2.

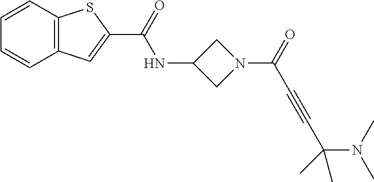 [SMILES: CN(C)C(C)(C)C#CC(=O)N1CC(NC(=O)c2cc3ccccc3s2)C1]

Example 225: N-(1-(4-(dimethylamino)-4-methylpent-2-ynoyl)azetidin-3-yl)benzo[b]thiophene-2-carboxamide (XS224-156). Example 225 was synthesized following similar procedure for preparing example 79. White solid, 26% yield ¹H NMR (400 MHz, Methanol-d₄) δ 7.99 (s, 1H), 7.90 (t, J=6.6 Hz, 2H), 7.43 (p, J=6.7 Hz, 2H), 4.83-4.76 (m, 1H), 4.65 (t, J=8.7 Hz, 1H), 4.42 (t, J=9.5 Hz, 1H), 4.36-4.30 (m, 1H), 4.20-4.09 (m, 1H), 2.99 (s, 6H), 1.76 (s, 6H). MS (ESI) [M+H]⁺=370.2.

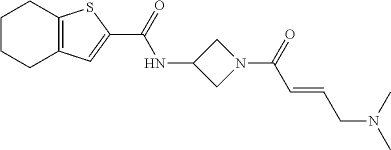 [SMILES: CN(C)C/C=C/C(=O)N1CC(NC(=O)c2cc3c(s2)CCCC3)C1]

Example 226: (E)-N-(1-(4-(dimethylamino)but-2-enoyl)azetidin-3-yl)-4,5,6,7-tetrahydrobenzo[b]thiophene-2-carboxamide (XS224-157). Example 226 was synthesized following similar procedure for preparing example 79. White solid, 25% yield ¹H NMR (400 MHz, Methanol-d₄) δ 7.31 (s, 1H), 6.71-6.57 (m, 1H), 6.43 (d, J=15.3 Hz, 1H), 4.73-4.62 (m, 1H), 4.58 (t, J=8.5 Hz, 1H), 4.32 (t, J=9.4 Hz, 1H), 4.26-4.16 (m, 1H), 4.09-3.97 (m, 1H), 3.87 (d, J=7.0 Hz, 2H), 2.82 (s, 6H), 2.69 (t, J=6.3 Hz, 2H), 2.53 (t, J=5.8 Hz, 2H), 1.81-1.64 (m, 4H). MS (ESI) [M+H]⁺=348.3.

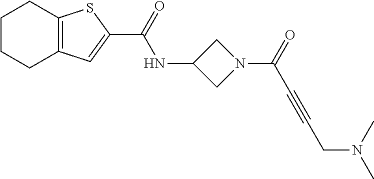 [SMILES: CN(C)CC#CC(=O)N1CC(NC(=O)c2cc3c(s2)CCCC3)C1]

Example 227: N-(1-(4-(dimethylamino)but-2-ynoyl)azetidin-3-yl)-4,5,6,7-tetrahydrobenzo[b]thiophene-2-carboxamide (XS224-158). Example 227 was synthesized following similar procedure for preparing example 79. White solid, 23% yield ¹H NMR (400 MHz, Methanol-d₄) δ 7.37 (s, 1H), 4.79-4.68 (m, 1H), 4.60 (t, 0.1=8.7 Hz, 1H), 4.40-4.30 (m, 3H), 4.30-4.21 (m, 1H), 4.14-4.02 (m, 1H), 2.98 (s, 6H), 2.77 (t, J=6.0 Hz, 2H), 2.61 (t, J=6.0 Hz, 2H), 1.93-1.72 (m, 4H). MS (ESI) [M+H]⁺=346.2.

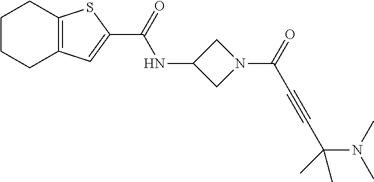 [SMILES: CN(C)C(C)(C)C#CC(=O)N1CC(NC(=O)c2cc3c(s2)CCCC3)C1]

Example 228: N-(1-(4-(dimethylamino)-4-methylpent-2-ynoyl)azetidin-3-yl)-4,5,6,7-tetrahydrobenzo[b]thiophene-2-carboxamide (XS224-159). Example 228 was synthesized following similar procedure for preparing example 79. White solid, 29% yield ¹H NMR (400 MHz, Methanol-d4) δ 7.36 (s, 1H), 4.74 (p, J=6.5, 5.9 Hz, 1H), 4.59 (t, J=8.6 Hz, 1H), 4.36 (t, J=9.5 Hz, 1H), 4.29-4.20 (m, 1H), 4.12-4.03 (m, 1H), 2.98 (s, 6H), 2.75 (t, J=6.4 Hz, 2H), 2.60 (t, J=6.0 Hz, 2H), 1.89-1.77 (m, 4H), 1.75 (s, 6H). MS (ESI) [M+H]⁺=374.2.

### Procedures for the Synthesis of AMPK Based Bivalent Compounds

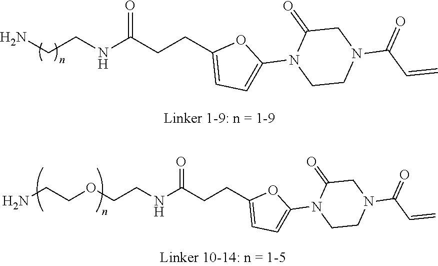

The linkers 1-14 were synthesized following the reported procedures (Liu et al., 2022)

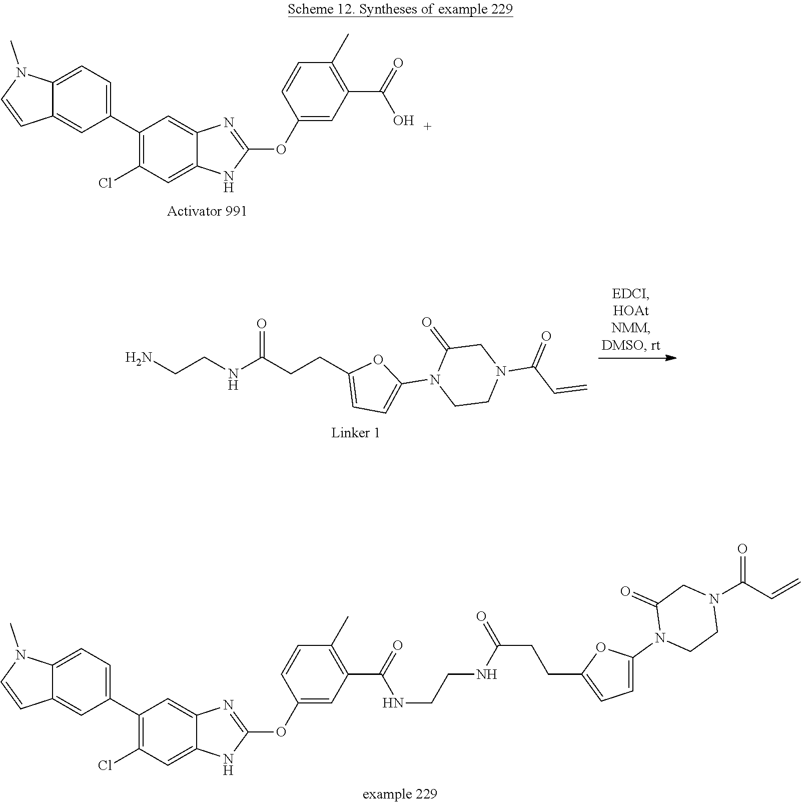 [SMILES: Cc1ccc(Oc2nc3cc(-c4ccc5c(ccn5C)c4)c(Cl)cc3[nH]2)cc1C(=O)O ; C=CC(=O)N1CCN(c2ccc(CCC(=O)NCCN)o2)C(=O)C1 ; C=CC(=O)N1CCN(c2ccc(CCC(=O)NCCNC(=O)c3cc(Oc4nc5cc(-c6ccc7c(ccn7C)c6)c(Cl)cc5[nH]4)ccc3C)o2)C(=O)C1]

Example 229: N-(2-(3-(5-(4-acryloyl-2-oxopiperazin-1-yl)furan-2-yl)propanamido)ethyl)-5-((6-chloro-5-(1-methyl-1H-indol-5-yl)-1H-benzo[d]imidazol-2-yl)oxy)-2-methylbenzamide(XF137-81). To a solution of activator 991 (prepared according to previous published paper) (Ngoei et al., 2018) (21 mg, 0.048 mmol) in DMSO (1 mL) were added linker 1 (16.3 mg, 0.048 mmol, 1.0 equiv), EDCI (14 mg, 0.072 mmol, 1.5 equiv), HOAt (10 mg, 0.072 mmol, 1.5 equiv), and NMM (15 mg, 0.14 mmol, 3.0 equiv). After being stirred for 3 h at rt, the resulting mixture was purified by preparative HPLC to afford titled compound as brown solid. (6.7 mg, yield 19%). ¹H NMR (400 MHz, CD₃OD) δ 7.62-7.48 (m, 3H), 7.48-7.35 (m, 5H), 7.27-7.18 (m, 2H), 6.75-6.59 (m, 1H), 6.30-6.25 (m, 1H), 6.16 (d, J=3.1 Hz, 1H), 6.00 (d, J=3.2 Hz, 1H), 5.80 (d, J=10.5 Hz, 1H), 4.42-4.21 (m, 2H), 3.91-3.82 (m, 5H), 3.81-3.69 (m, 2H), 3.53-3.38 (m, 4H), 2.80 (t, J=7.5 Hz, 2H), 2.47 (s, 5H). MS (ESI) [M+H]⁺=748.3.

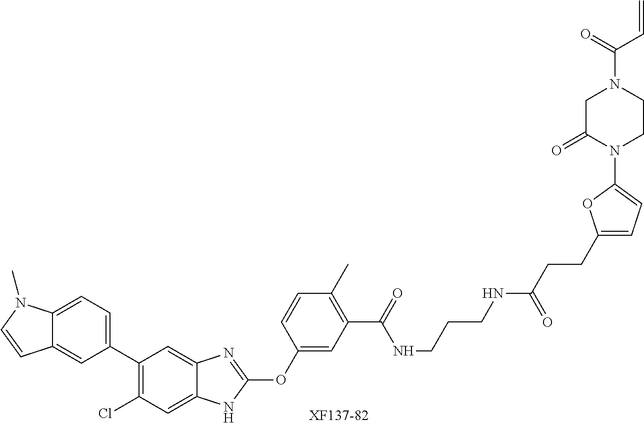 [SMILES: C=CC(=O)N1CCN(c2ccc(CCC(=O)NCCCNC(=O)c3cc(Oc4nc5cc(-c6ccc7c(ccn7C)c6)c(Cl)cc5[nH]4)ccc3C)o2)C(=O)C1]

Example 230: N-(3-(3-(5-(4-acryloyl-2-oxopiperazin-1-yl)furan-2-yl)propanamido)propyl)-5-((6-chloro-5-(1-methyl-1H-indol-5-yl)-1H-benzo[d]imidazol-2-yl)oxy)-2-methylbenzamide (XF137-82). Example 230 was synthesized following the standard procedure for preparing example 229 from activator 991 (28 mg, 0.06 mmol) and linker 2 (23 mg, 0.06 mmol, 1.0 equiv). Brown solid (15.4 mg, yield 34%). ¹H NMR (400 MHz, CD₃OD) δ 7.61-7.47 (m, 3H), 7.45-7.34 (m, 5H), 7.28-7.14 (m, 2H), 6.83-6.61 (m, 1H), 6.28 (dd, J=16.8, 1.9 Hz, 1H), 6.21 (d, J=3.2 Hz, 1H), 6.09 (d, J=3.2 Hz, 1H), 5.80 (dd, J=10.5, 1.9 Hz, 1H), 4.45-4.27 (m, 2H), 3.93 (t, J=5.3 Hz, 2H), 3.84 (d, J=9.3 Hz, 5H), 3.38-3.34 (m, 2H), 3.27 (d, J=6.8 Hz, 2H), 2.88 (t, J=7.3 Hz, 2H), 2.48 (d, J=11.3 Hz, 5H), 1.76 (t, J=6.8 Hz, 2H). MS (ESI) [M+H]⁺=762.3.

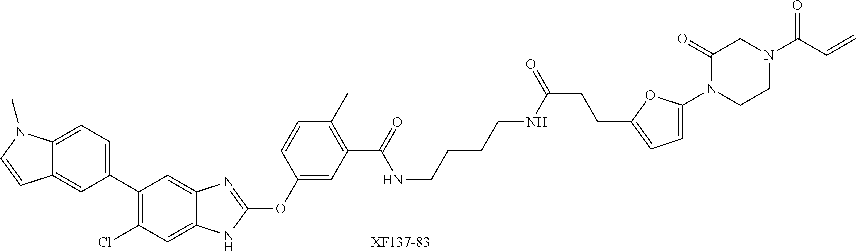 [SMILES: C=CC(=O)N1CCN(c2ccc(CCC(=O)NCCCCNC(=O)c3cc(Oc4nc5cc(-c6ccc7c(ccn7C)c6)c(Cl)cc5[nH]4)ccc3C)o2)C(=O)C1]

Example 231: N-(4-(3-(5-(4-acryloyl-2-oxopiperazin-1-yl)furan-2-yl)propanamido)butyl)-5-((6-chloro-5-(1-methyl-1H-indol-5-yl)-1H-benzo[d]imidazol-2-yl)oxy)-2-methylbenzamide (XF137-83). Example 231 was synthesized following the standard procedure for preparing example 229 from activator 991 (21 mg, 0.048 mmol) and liner 3 (17 mg, 0.048 mmol, 1.0 equiv). Brown solid (7.6 mg, yield 20%). ¹H NMR (400 MHz, CD₃OD) δ 7.57 (d, J=2.5 Hz, 3H), 7.51-7.32 (m, 5H), 7.23 (d, J=8.3 Hz, 2H), 6.84-6.57 (m, 1H), 6.29 (dd, J=16.8, 1.9 Hz, 1H), 6.20 (d, J=3.2 Hz, 1H), 6.08 (d, J=3.2 Hz, 1H), 5.81 (dd, J=10.6, 1.9 Hz, 1H), 4.44-4.24 (m, 2H), 3.95-3.89 (m, 2H), 3.87-3.75 (m, 5H), 3.47-3.35 (m, 2H), 3.21 (t, J=6.4 Hz, 2H), 2.87 (t, J=7.3 Hz, 2H), 2.47 (s, 5H), 1.73-1.47 (m, 4H). MS (ESI) [M+H]⁺=776.3.

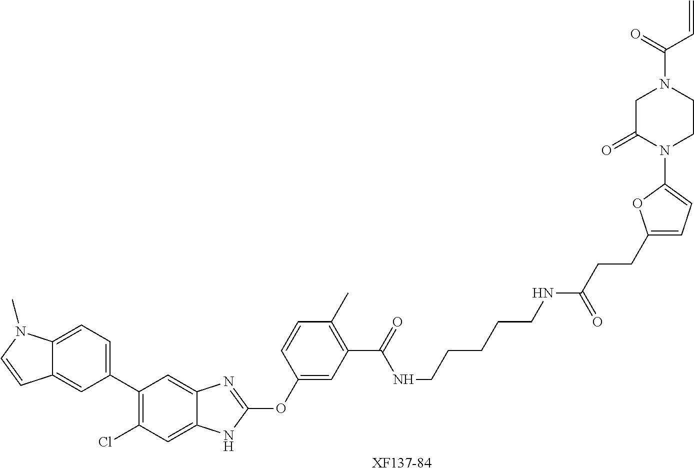 [SMILES: C=CC(=O)N1CCN(c2ccc(CCC(=O)NCCCCCNC(=O)c3cc(Oc4nc5cc(-c6ccc7c(ccn7C)c6)c(Cl)cc5[nH]4)ccc3C)o2)C(=O)C1]

Example 232: N-(5-(3-(5-(4-acryloyl-2-oxopiperazin-1-yl)furan-2-yl)propanamido)pentyl)-5-((6-chloro-5-(1-methyl-1H-indol-5-yl)-1H-benzo[d]imidazol-2-yl)oxy)-2-methylbenzamide (XF137-84). Example 232 was synthesized following the standard procedure for preparing example 229 from activator 991 (21 mg, 0.048 mmol) and liner 4 (18 mg, 0.048 mmol, 1.0 equiv). Brown solid (11.1 mg, yield 29%). ¹H NMR (400 MHz, CD₃OD) δ7.62-7.52 (m, 2H), 7.50-7.34 (m, 6H), 7.27-7.17 (m, 2H), 6.84-6.60 (m, 1H), 6.29 (dd, J=16.8, 1.8 Hz, 1H), 6.23 (d, J=3.1 Hz, 1H), 6.08 (d, J=3.0 Hz, 1H), 5.88-5.72 (m, 1H), 4.47-4.28 (m, 2H), 4.03-3.90 (m, 2H), 3.86 (d, J=1.1 Hz, 5H), 3.47-3.35 (m, 2H), 3.17 (t, J=6.8 Hz, 2H), 2.87 (t, J=7.3 Hz, 2H), 2.46 (s, 5H), 1.70-1.57 (m, 2H), 1.57-1.47 (m, 2H), 1.47-1.30 (m, 2H). MS (ESI) [M+H]⁺=790.3.

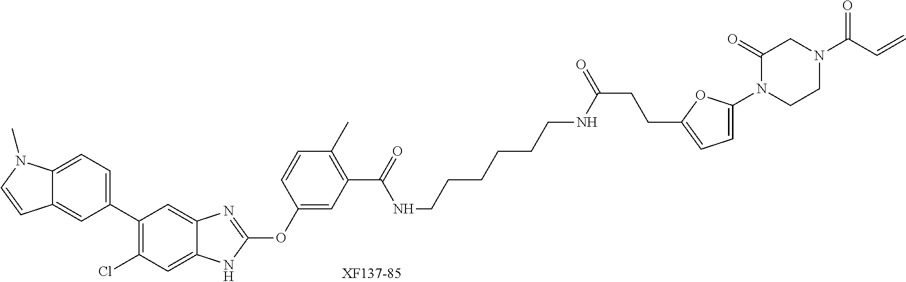 [SMILES: C=CC(=O)N1CCN(c2ccc(CCC(=O)NCCCCCCNC(=O)c3cc(Oc4nc5cc(-c6ccc7c(ccn7C)c6)c(Cl)cc5[nH]4)ccc3C)o2)C(=O)C1]

Example 233: N-(6-(3-(5-(4-acryloyl-2-oxopiperazin-1-yl)furan-2-yl)propanamido)hexyl)-5-((6-chloro-5-(1-methyl-1H-indol-5-yl)-1H-benzo[d]imidazol-2-yl)oxy)-2-methylbenzamide (XF137-85). Example 233 was synthesized following the standard procedure for preparing example 229 from activator 991 (21 mg, 0.048 mmol) and linker 5 (18.7 mg, 0.048 mmol, 1.0 equiv). Compound XF137-85 was obtained as brown solid (9.1 mg, yield 24%). ¹H NMR (400 MHz, CD₃OD) δ 7.58 (d, J=3.0 Hz, 2H), 7.52-7.32 (m, 6H), 7.23 (d, J=8.3 Hz, 2H), 6.85-6.61 (m, 1H), 6.29 (dd, J=16.7, 2.0 Hz, 1H), 6.22 (d, J=3.2 Hz, 1H), 6.08 (d, J=3.2 Hz, 1H), 5.82 (dd, J=10.5, 1.9 Hz, 1H), 4.47-4.26 (m, 2H), 4.03-3.89 (m, 2H), 3.89-3.74 (m, 5H), 3.43-3.34 (m, 2H), 3.15 (t, J=6.8 Hz, 2H), 2.88 (t, J=7.3 Hz, 2H), 2.47 (s, 5H), 1.69-1.55 (m, 2H), 1.54-1.27 (m, 6H). MS (ESI) [M+H]⁺=804.3.

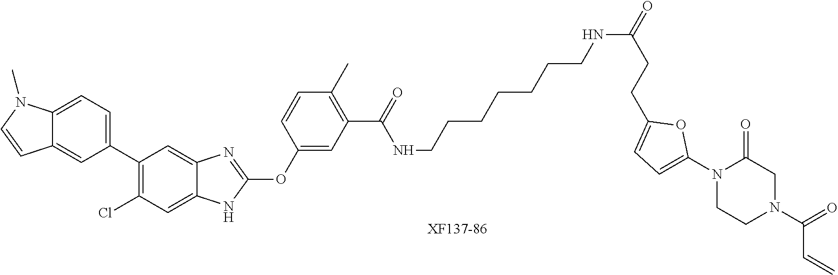 [SMILES: C=CC(=O)N1CCN(c2ccc(CCC(=O)NCCCCCCCNC(=O)c3cc(Oc4nc5cc(-c6ccc7c(ccn7C)c6)c(Cl)cc5[nH]4)ccc3C)o2)C(=O)C1]

Example 234: N-(7-(3-(5-(4-acryloyl-2-oxopiperazin-1-yl)furan-2-yl)propanamido)heptyl)-5-((6-chloro-5-(1-methyl-1H-indol-5-yl)-1H-benzo[d]imidazol-2-yl)oxy)-2-methylbenzamide (XF137-86). Example 234 was synthesized following the standard procedure for preparing example 229 from activator 991 (21 mg, 0.048 mmol) and linker 6 (19 mg, 0.048 mmol, 1.0 equiv). Brown solid (12.6 mg, yield 32%). ¹H NMR (400 MHz, CD₃OD) δ 7.63-7.52 (m, 2H), 7.49-7.31 (m, 6H), 7.28-7.16 (m, 2H), 6.88-6.64 (m, 1H), 6.30 (dd, J=16.8, 1.9 Hz, 1H), 6.22 (d, J=3.3 Hz, 1H), 6.08 (d, J=3.2 Hz, 1H), 5.82 (dd, J=10.6, 1.9 Hz, 1H), 4.47-4.31 (m, 2H), 4.06-3.94 (m, 2H), 3.86 (s, 5H), 3.41-3.36 (m, 2H), 3.14 (t, J=6.9 Hz, 2H), 2.89 (t, J=7.3 Hz, 2H), 2.52-2.40 (m, 5H), 1.62 (q, J=7.1 Hz, 2H), 1.52-1.21 (m, 8H). MS (ESI) [M+H]⁺=818.4.

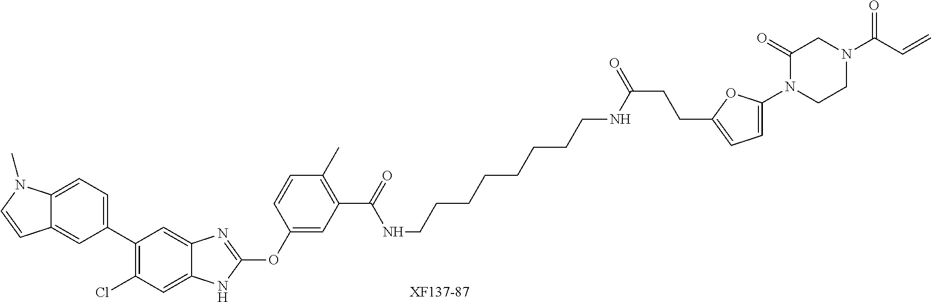 [SMILES: C=CC(=O)N1CCN(c2ccc(CCC(=O)NCCCCCCCCNC(=O)c3cc(Oc4nc5cc(-c6ccc7c(ccn7C)c6)c(Cl)cc5[nH]4)ccc3C)o2)C(=O)C1]

Example 235: N-(8-(3-(5-(4-acryloyl-2-oxopiperazin-1-yl)furan-2-yl)propanamido)octyl)-5-((6-chloro-5-(1-methyl-1H-indol-5-yl)-1H-benzo[d]imidazol-2-yl)oxy)-2-methylbenzamide (XF137-87). Example 235 was synthesized following the standard procedure for preparing Example 229 from activator 991 (21 mg, 0.048 mmol) and linker 7 (20 mg, 0.048 mmol, 1.0 equiv). Brown solid (9.6 mg, yield 24%). ¹H NMR (400 MHz, CD₃OD) δ ¹H NMR (400 MHz, MeOD) δ 7.62-7.52 (m, 2H), 7.49-7.36 (m, 6H), 7.28-7.18 (m, 2H), 6.82-6.64 (m, 1H), 6.35-6.26 (m, 1H), 6.25-6.21 (m, 1H), 6.08 (d, J=3.2 Hz, 1H), 5.82 (dt, J=10.6, 1.3 Hz, 1H), 4.40 (d, J=26.3 Hz, 2H), 4.02-3.92 (m, 2H), 3.89-3.78 (m, 5H), 3.42-3.35 (m, 2H), 3.13 (t, J=7.0 Hz, 2H), 2.89 (t, J=7.3 Hz, 2H), 2.55-2.39 (m, 5H), 1.62 (q, J=7.2 Hz, 2H), 1.52-1.24 (m, 10H). MS (ESI) [M+H]⁺=832.2.

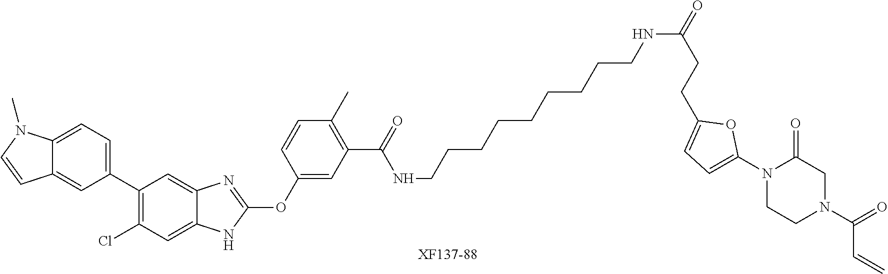 [SMILES: C=CC(=O)N1CCN(c2ccc(CCC(=O)NCCCCCCCCCNC(=O)c3cc(Oc4nc5cc(-c6ccc7c(ccn7C)c6)c(Cl)cc5[nH]4)ccc3C)o2)C(=O)C1]

Example 236: N-(9-(3-(5-(4-acryloyl-2-oxopiperazin-1-yl)furan-2-yl)propanamido)nonyl)-5-((6-chloro-5-(1-methyl-1H-indol-5-yl)-1H-benzo[d]imidazol-2-yl)oxy)-2-methylbenzamide (XF137-88). Example 236 was synthesized following the standard procedure for preparing example 229 from activator 991 (21 mg, 0.048 mmol) and linker 8 (21 mg, 0.048 mmol, 1.0 equiv). Brown solid (12.9 mg, yield 32%). ¹H NMR (400 MHz, CD₃OD) δ 7.54 (d, J=17.1 Hz, 2H), 7.43-7.28 (m, 6H), 7.28-7.10 (m, 2H), 6.73 (d, J=10.8 Hz, 1H), 6.30-6.28 (m, 1H), 6.22 (d, J=3.2 Hz, 1H), 6.11-5.98 (m, 1H), 5.81 (dd, J=10.5, 2.0 Hz, 1H), 4.46-4.26 (m, 2H), 3.93 (t, J=5.4 Hz, 2H), 3.87-3.76 (m, 5H), 3.41-3.34 (m, 2H), 3.12 (t, J=7.0 Hz, 2H), 2.88 (t, J=7.3 Hz, 2H), 2.51-2.38 (m, 5H), 1.61 (q, J=7.2 Hz, 2H), 1.50-1.22 (m, 12H). MS (ESI) [M+H]⁺=846.4.

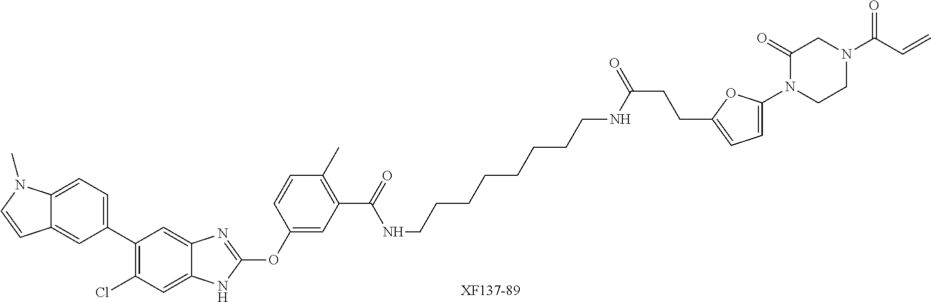 [SMILES: C=CC(=O)N1CCN(c2ccc(CCC(=O)NCCCCCCCCNC(=O)c3cc(Oc4nc5cc(-c6ccc7c(ccn7C)c6)c(Cl)cc5[nH]4)ccc3C)o2)C(=O)C1]

Example 237: N-(10-(3-(5-(4-acryloyl-2-oxopiperazin-1-yl)furan-2-yl)propanamido)decyl)-5-((6-chloro-5-(1-methyl-1H-indol-5-yl)-1H-benzo[d]imidazol-2-yl)oxy)-2-methylbenzamide (XF137-89). Example 237 was synthesized following the standard procedure for preparing example 229 from activator 991 (21 mg, 0.048 mmol) and linker 9 (21 mg, 0.048 mmol, 1.0 equiv). Brown solid (12.8 mg, yield 31%). ¹H NMR (400 MHz, CD₃OD) δ 7.62-7.47 (m, 2H), 7.47-7.29 (m, 6H), 7.27-7.19 (m, 2H), 6.82-6.65 (m, 1H), 6.35-6.25 (m, 1H), 6.24-6.17 (m, 1H), 6.08 (d, J=3.2 Hz, 1H), 5.88-5.67 (m, 1H), 4.48-4.28 (m, 2H), 3.94 (t, J=5.1 Hz, 2H), 3.84 (d, J=1.4 Hz, 5H), 3.40-3.34 (m, 2H), 3.12 (t, J=7.0 Hz, 2H), 2.89 (t, J=7.3 Hz, 2H), 2.46 (d, J=7.8 Hz, 5H), 1.62 (q, J=7.1 Hz, 2H), 1.50-1.17 (m, 14H). MS (ESI) [M+H]⁺=860.3.

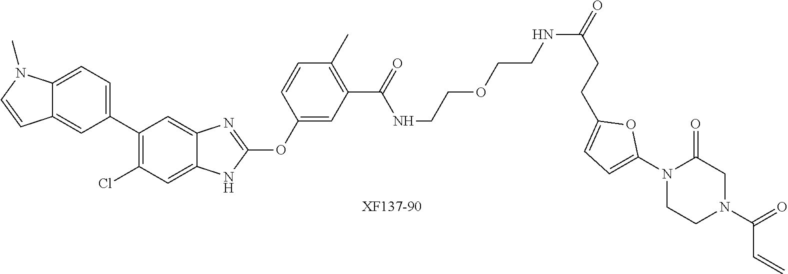 [SMILES: C=CC(=O)N1CCN(c2ccc(CCC(=O)NCCOCCNC(=O)c3cc(Oc4nc5cc(-c6ccc7c(ccn7C)c6)c(Cl)cc5[nH]4)ccc3C)o2)C(=O)C1]

Example 238: N-(2-(2-(3-(5-(4-acryloyl-2-oxopiperazin-1-yl)furan-2-yl)propanamido)ethoxy)ethyl)-5-((6-chloro-5-(1-methyl-1H-indol-5-yl)-1H-benzo[d]imidazol-2-yl)oxy)-2-methylbenzamide (XF137-90). Example 238 was synthesized following the standard procedure for preparing example 229 from activator 991 (21 mg, 0.048 mmol) and linker 10 (18.1 mg, 0.048 mmol, 1.0 equiv). Brown solid (16 mg, yield 42%). ¹H NMR (400 MHz, CD₃OD) δ 7.60-7.52 (m, 2H), 7.51-7.36 (m, 6H), 7.26-7.18 (m, 2H), 6.82-6.59 (m, 1H), 6.28 (dd, J=16.8, 1.9 Hz, 1H), 6.20 (d, J=3.2 Hz, 1H), 6.03 (d, J=3.2 Hz, 1H), 5.80 (dd, J=10.6, 1.9 Hz, 1H), 4.41-4.26 (m, 2H), 3.90 (t, J=5.4 Hz, 2H), 3.85 (s, 3H), 3.78 (s, 2H), 3.67-3.49 (m, 6H), 3.38-3.35 (m, 2H), 2.79 (t, J=7.4 Hz, 2H), 2.47 (s, 3H), 2.42 (t, J=7.4 Hz, 2H). MS (ESI) [M+H]⁺=792.2.

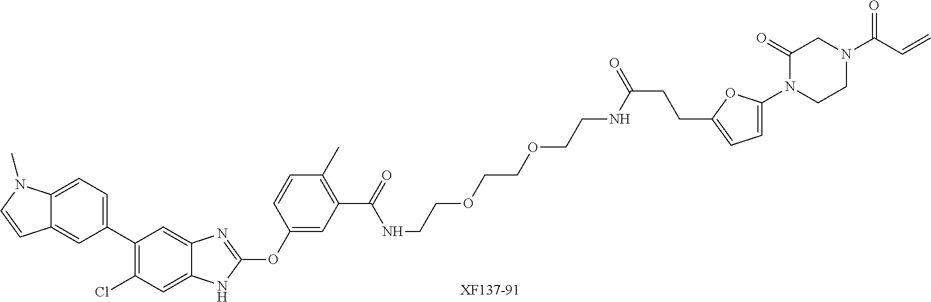 [SMILES: C=CC(=O)N1CCN(c2ccc(CCC(=O)NCCOCCOCCNC(=O)c3cc(Oc4nc5cc(-c6ccc7c(ccn7C)c6)c(Cl)cc5[nH]4)ccc3C)o2)C(=O)C1]

Example 239: N-(2-(2-(2-(3-(5-(4-acryloyl-2-oxopiperazin-1-yl)furan-2-yl)propanamido)ethoxy)ethoxy)ethyl)-5-((6-chloro-5-(1-methyl-1H-indol-5-yl)-1H-benzo[d]imidazol-2-yl)oxy)-2-methylbenzamide (XF137-91). Example 239 was synthesized following the standard procedure for preparing example 229 from activator 991 (21 mg, 0.048 mmol) and linker 11 (20.2 mg, 0.048 mmol, 1.0 equiv). Brown solid (21.8 mg, yield 54%). ¹H NMR (400 MHz, CD₃OD) δ 7.64-7.53 (m, 2H), 7.52-7.33 (m, 6H), 7.23 (d, J=8.7 Hz, 2H), 6.80-6.61 (m, 1H), 6.28 (dd, J=16.8, 1.9 Hz, 1H), 6.22-6.13 (m, 1H), 6.07 (d, J=3.2 Hz, 1H), 5.81 (dt, J=10.6, 1.6 Hz, 1H), 4.37 (d, J=25.5 Hz, 2H), 3.92 (t, J=5.4 Hz, 2H), 3.87-3.74 (m, 5H), 3.70-3.52 (m, 8H), 3.47 (t, J=5.5 Hz, 2H), 3.31-3.26 (m, 2H), 2.85 (t, J=7.4 Hz, 2H), 2.50-2.39 (m, 5H). MS (ESI) [M+H]⁺=836.3.

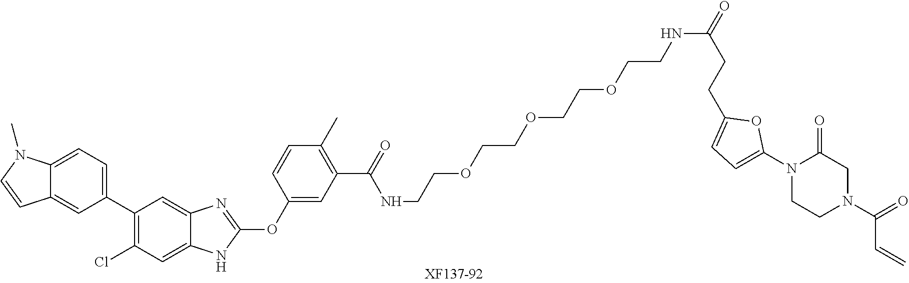 [SMILES: C=CC(=O)N1CCN(c2ccc(CCC(=O)NCCOCCOCCOCCNC(=O)c3cc(Oc4nc5cc(-c6ccc7c(ccn7C)c6)c(Cl)cc5[nH]4)ccc3C)o2)C(=O)C1]

Example 240: N-(15-(5-(4-acryloyl-2-oxopiperazin-1-yl)furan-2-yl)-13-oxo-3,6,9-trioxa-12-azapentadecyl)-5-((6-chloro-5-(1-methyl-1H-indol-5-yl)-1H-benzo[d]imidazol-2-yl)oxy)-2-methylbenzamide (XF137-92). Example 240 was synthesized following the standard procedure for preparing example 229 from activator 991 (21 mg, 0.048 mmol) and linker 12 (22 mg, 0.048 mmol, 1.0 equiv). Brown solid (22.9 mg, yield 61%). ¹H NMR (400 MHz, CD₃OD) δ 7.59 (d, J=7.6 Hz, 2H), 7.53-7.35 (m, 6H), 7.27-7.19 (m, 2H), 6.80-6.64 (m, 1H), 6.29 (dd, J=16.7, 1.9 Hz, 1H), 6.22 (d, J=3.3 Hz, 1H), 6.08 (d, J=3.2 Hz, 1H), 5.81 (dd, J=10.7, 1.9 Hz, 1H), 4.44-4.30 (m, 2H), 3.97-3.88 (m, 2H), 3.87-3.75 (m, 5H), 3.70-3.49 (m, 12H), 3.45 (t, J=5.4 Hz, 2H), 3.30 (d, J=5.5 Hz, 2H), 2.88 (t, J=7.4 Hz, 2H), 2.51-2.44 (m, 5H). MS (ESI) [M+H]⁺=880.3.

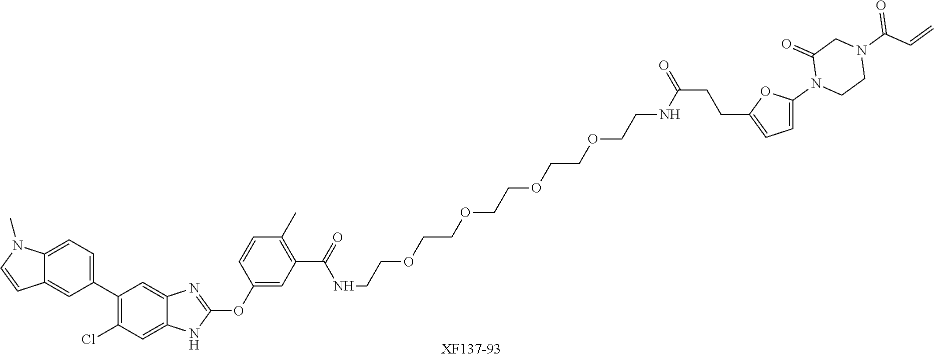 [SMILES: C=CC(=O)N1CCN(c2ccc(CCC(=O)NCCOCCOCCOCCOCCNC(=O)c3cc(Oc4nc5cc(-c6ccc7c(ccn7C)c6)c(Cl)cc5[nH]4)ccc3C)o2)C(=O)C1]

Example 241: N-(18-(5-(4-acryloyl-2-oxopiperazin-1-yl)furan-2-yl)-16-oxo-3,6,9,12-tetraoxa-15-azaoctadecyl)-5-((6-chloro-5-(1-methyl-1H-indol-5-yl)-1H-benzo[d]imidazol-2-yl)oxy)-2-methylbenzamide (XF137-93). Example 241 was synthesized following the standard procedure for preparing example 229 from activator 991 (21 mg, 0.048 mmol) and linker 13 (24 mg, 0.048 mmol, 1.0 equiv). Brown solid (19.8 mg, yield 45%). ¹H NMR (400 MHz, CD₃OD) δ 7.58 (d, J=5.8 Hz, 2H), 7.51-7.40 (m, 6H), 7.28-7.18 (m, 2H), 6.83-6.65 (m, 1H), 6.29 (dd, J=16.8, 2.0 Hz, 1H), 6.22 (d, J=3.2 Hz, 1H), 6.09 (d, J=3.2 Hz, 1H), 5.81 (dd, J=10.6, 1.9 Hz, 1H), 4.47-4.31 (m, 2H), 3.97-3.88 (m, 2H), 3.88-3.75 (m, 3H), 3.70-3.51 (m, 18H), 3.47 (t, J=5.5 Hz, 2H), 3.38-3.36 (m, 2H), 2.89 (t, J=7.4 Hz, 2H), 2.55-2.40 (m, 5H). MS (ESI) [M+H]⁺=924.4.

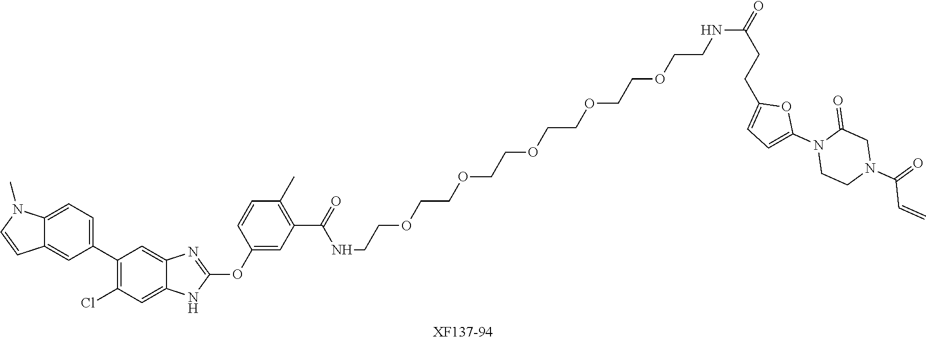 [SMILES: C=CC(=O)N1CCN(c2ccc(CCC(=O)NCCOCCOCCOCCOCCOCCNC(=O)c3cc(Oc4nc5cc(-c6ccc7c(ccn7C)c6)c(Cl)cc5[nH]4)ccc3C)o2)C(=O)C1]

Example 242: N-(21-(5-(4-acryloyl-2-oxopiperazin-1-yl)furan-2-yl)-19-oxo-3,6,9,12,15-pentaoxa-18-azahenicosyl)-5-((6-chloro-5-(1-methyl-1H-indol-5-yl)-1H-benzo[d]imidazol-2-yl)oxy)-2-methylbenzamide (XF137-94). Example 242 was synthesized following the standard procedure for preparing example 229 from activator 991 (21 mg, 0.048 mmol) and linker 14 (27 mg, 0.048 mmol, 1.0 equiv). Brown solid (16.8 mg, yield 36%). ¹H NMR (400 MHz, CD₃OD) δ 7.59 (d, J=5.8 Hz, 2H), 7.52-7.41 (m, 6H), 7.28-7.18 (m, 2H), 6.82-6.66 (m, 1H), 6.29 (dd, J=16.7, 2.0 Hz, 1H), 6.23 (d, J=3.2 Hz, 1H), 6.10 (d, J=3.2 Hz, 1H), 5.82 (dd, J=10.6, 1.9 Hz, 1H), 4.48-4.32 (m, 2H), 3.97-3.88 (m, 2H), 3.86-3.74 (m, 5H), 3.70-3.51 (m, 20H), 3.48 (t, J=5.5 Hz, 2H), 3.39-3.36 (m, 2H), 2.90 (t, J=7.4 Hz, 2H), 2.55-2.42 (m, 5H). MS (ESI) [M+H]⁺=968.5.

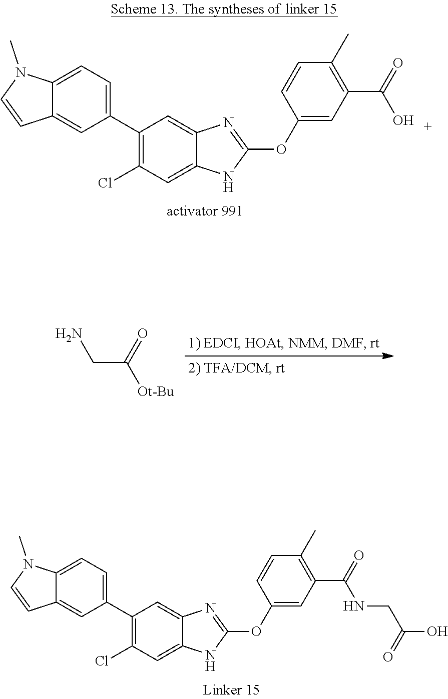 [SMILES: Cc1ccc(Oc2nc3cc(-c4ccc5c(ccn5C)c4)c(Cl)cc3[nH]2)cc1C(=O)O ; CC(C)(C)OC(=O)CN ; Cc1ccc(Oc2nc3cc(-c4ccc5c(ccn5C)c4)c(Cl)cc3[nH]2)cc1C(=O)NCC(=O)O]

Linker 15: (5-((6-chloro-5-(1-methyl-1H-indol-5-yl)-1H-benzo[d]imidazol-2-yl)oxy)-2-methylbenzoyl)glycine To a solution of activator 991 (50 mg, 0.12 mmol) in DMF (1 mL) were added tert-butyl (2-aminoethyl)carbamate (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred for overnight at rt, the resulting mixture was purified by prep-HPLC to afford crude product. The crude product was dissolved in DCM (1 mL) and TFA (1 mL). After being stirred at rt for 30 min, the resulting mixture was purified by prep-HPLC to afford compound titled compound as yellow solid (57 mg, yield 81%). MS (ESI) [M+H]⁺=489.3. Linker 16-27 were synthesized following the same procedure for preparing linker 15.

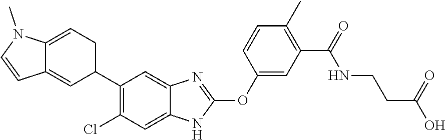 [SMILES: Cc1ccc(Oc2nc3cc(C4C=c5ccn(C)c5=CC4)c(Cl)cc3[nH]2)cc1C(=O)NCCC(=O)O]

Linker 16: 3-(5-((6-chloro-5-(1-methyl-1H-indol-5-yl)-1H-benzo[d]imidazol-2-yl)oxy)-2-methylbenzamido)propanoic acid MS (ESI) [M+H]⁺=503.2.

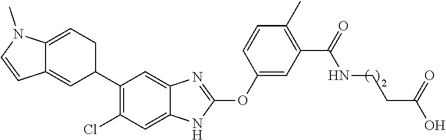 [SMILES: Cc1ccc(Oc2nc3cc(C4C=c5ccn(C)c5=CC4)c(Cl)cc3[nH]2)cc1C(=O)NCCC(=O)O]

Linker 17: 4-(5-((6-chloro-5-(1-methyl-1H-indol-5-yl)-1H-benzo[d]imidazol-2-yl)oxy)-2-methylbenzamido)butanoic acid MS (ESI) [M+H]⁺=517.4.

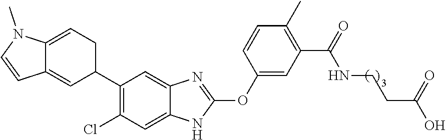 [SMILES: Cc1ccc(Oc2nc3cc(C4C=c5ccn(C)c5=CC4)c(Cl)cc3[nH]2)cc1C(=O)NCCC(=O)O]

Linker 18: 5-(5-((6-chloro-5-(1-methyl-1H-indol-5-yl)-1H-benzo[d]imidazol-2-yl)oxy)-2-methylbenzamido)pentanoic acid MS (ESI) [M+H]⁺=531.3.

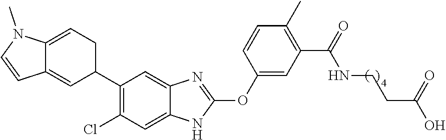 [SMILES: Cc1ccc(Oc2nc3cc(C4C=c5ccn(C)c5=CC4)c(Cl)cc3[nH]2)cc1C(=O)NCCC(=O)O]

Linker 19: 6-(5-((6-chloro-5-(1-methyl-1H-indol-5-yl)-1H-benzo[d]imidazol-2-yl)oxy)-2-methylbenzamido)hexanoic acid MS (ESI) [M+H]⁺=545.3.

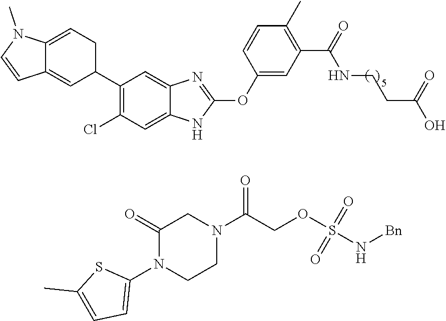 [SMILES: Cc1ccc(Oc2nc3cc(C4C=c5ccn(C)c5=CC4)c(Cl)cc3[nH]2)cc1C(=O)NCCC(=O)O ; Cc1ccc(N2CCN(C(=O)COS(=O)(=O)NCc3ccccc3)CC2=O)s1]

Example 110: (E)-N-(1-(4-(dimethylamino)but-2-enoyl)azetidin-3-yl)-5-methylthiophene-2-carboxamide (XS185-171). Example 110 was synthesized following similar procedure for preparing example 104. White solid, 15% yield ¹H NMR (400 MHz, DMSO-d₆) δ 8.54-8.35 (m, 1H), 7.34 (d, J=4.1 Hz, 4H), 7.29-7.20 (m, 1H), 6.67-6.49 (m, 2H), 4.81 (d, J=21.1 Hz, 2H), 4.25 (s, 2H), 4.19 (d, J=4.8 Hz, 2H), 3.91-3.82 (m, 2H), 3.82-3.69 (m, 2H), 2.36 (s, 3H).MS (ESI) [M+H]⁺=424.2

Linker 20: 7-(5-((6-chloro-5-(1-methyl-1H-indol-5-yl)-1H-benzo[d]imidazol-2-yl)oxy)-2-methylbenzamido)heptanoic acid MS (ESI) [M+H]⁺=559.4.

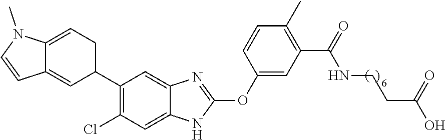 [SMILES: Cc1ccc(Oc2nc3cc(C4C=c5ccn(C)c5=CC4)c(Cl)cc3[nH]2)cc1C(=O)NCCC(=O)O]

Linker 21: 8-(5-((6-chloro-5-(1-methyl-1H-indol-5-yl)-1H-benzo[d]imidazol-2-yl)oxy)-2-methylbenzamido)octanoic acid MS (ESI) [M+H]⁺=573.3.

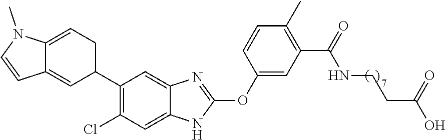 [SMILES: Cc1ccc(Oc2nc3cc(C4C=c5ccn(C)c5=CC4)c(Cl)cc3[nH]2)cc1C(=O)NCCC(=O)O]

Linker 22: 9-(5-((6-chloro-5-(1-methyl-1H-indol-5-yl)-1H-benzo[d]imidazol-2-yl)oxy)-2-methylbenzamido)nonanoic acid MS (ESI) [M+H]⁺=587.5.

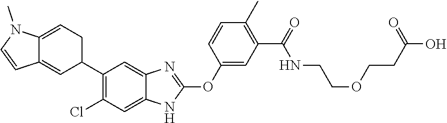 [SMILES: Cc1ccc(Oc2nc3cc(C4C=c5ccn(C)c5=CC4)c(Cl)cc3[nH]2)cc1C(=O)NCCOCCC(=O)O]

Linker 23: 3-(2-(5-((6-chloro-5-(1-methyl-1H-indol-5-yl)-1H-benzo[d]imidazol-2-yl)oxy)-2-methylbenzamido)ethoxy)propanoic acid MS (ESI) [M+H]⁺=548.6.

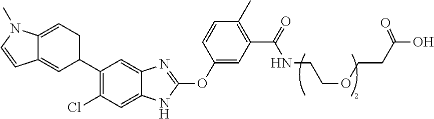

Linker 24: 3-(2-(2-(5-((6-chloro-5-(1-methyl-1H-indol-5-yl)-1H-benzo[d]imidazol-2-yl)oxy)-2-methylbenzamido)ethoxy)ethoxy)propanoic acid MS (ESI) [M+H]⁺=591.4.

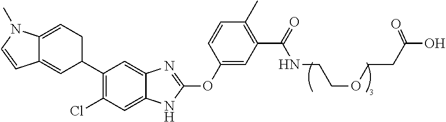

Linker 25: 1-(5-((6-chloro-5-(1-methyl-1H-indol-5-yl)-1H-benzo[d]imidazol-2-yl)oxy)-2-methylphenyl)-1-oxo-5,8,11-trioxa-2-azatetradecan-14-oic acid MS (ESI) [M+H]⁺=635.4.

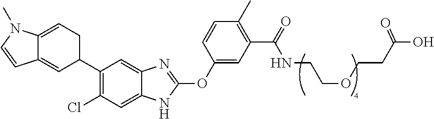

Linker 26: 1-(5-((6-chloro-5-(1-methyl-1H-indol-5-yl)-1H-benzo[d]imidazol-2-yl)oxy)-2-methylphenyl)-1-oxo-5,8,11,14-tetraoxa-2-azaheptadecan-17-oic acid MS (ESI) [M+H]⁺=679.4.

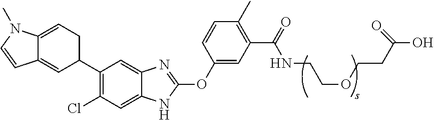

Linker 27: 1-(5-((6-chloro-5-(1-methyl-1H-indol-5-yl)-1H-benzo[d]imidazol-2-yl)oxy)-2-methylphenyl)-1-oxo-5,8,11,14,17-pentaoxa-2-azaicosan-20-oic acid MS (ESI) [M+H]⁺=723.3.

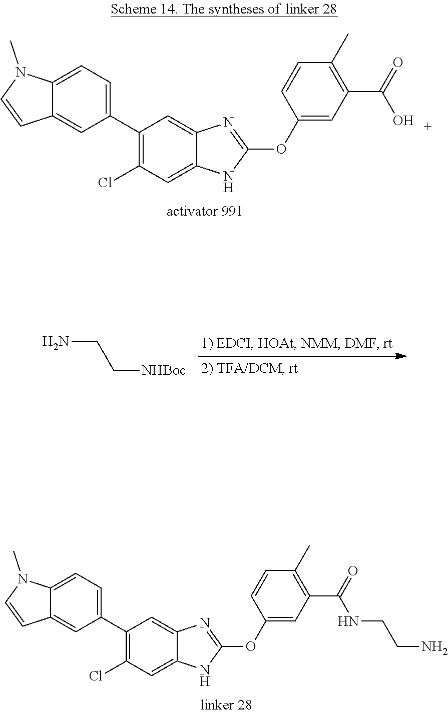 [SMILES: Cc1ccc(Oc2nc3cc(-c4ccc5c(ccn5C)c4)c(Cl)cc3[nH]2)cc1C(=O)O ; CC(C)(C)OC(=O)NCCN ; Cc1ccc(Oc2nc3cc(-c4ccc5c(ccn5C)c4)c(Cl)cc3[nH]2)cc1C(=O)NCCN]

Linker 28: N-(2-aminoethyl)-5-((6-chloro-5-(1-methyl-1H-indol-5-yl)-1H-benzo[d]imidazol-2-yl)oxy)-2-methylbenzamide (XH168-164). To a solution of activator 991 (50 mg, 0.12 mmol) in DMF (1 mL) were added tert-butyl (2-aminoethyl)carbamate (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred for overnight at rt, the resulting mixture was purified by preparative HPLC (10%-100% acetonitrile/0.1% TFA in H₂O) to afford crude product. The crude product was dissolved in DCM (1 mL) and TFA (1 mL). After being stirred at rt for 30 min, the resulting mixture was purified by preparative HPLC (10%-100% acetonitrile/0.1% TFA in H₂O) to afford compound titled compound as yellow solid (57 mg, yield 81%). MS (ESI) m/z 474.4 [M+H]⁺.

Linker 29-41 was synthesized following the same procedure for preparing linker 28.

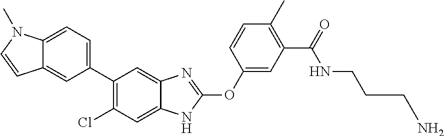 [SMILES: Cc1ccc(Oc2nc3cc(-c4ccc5c(ccn5C)c4)c(Cl)cc3[nH]2)cc1C(=O)NCCCN]

Linker 29: N-(3-aminopropyl)-5-((6-chloro-5-(1-methyl-1H-indol-5-yl)-1H-benzo[d]imidazol-2-yl)oxy)-2-methylbenzamide (XH168-165). Pink solid, 52 mg, 61% yield. MS (ESI) [M+H]⁺=488.3.

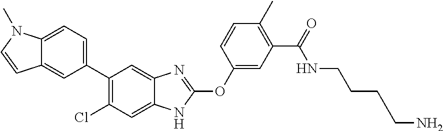 [SMILES: Cc1ccc(Oc2nc3cc(-c4ccc5c(ccn5C)c4)c(Cl)cc3[nH]2)cc1C(=O)NCCCCN]

Linker 30: N-(4-aminobutyl)-5-((6-chloro-5-(1-methyl-1H-indol-5-yl)-1H-benzo[d]imidazol-2-yl)oxy)-2-methylbenzamide (XH168-166). Pink solid, 59 mg, 67% yield. MS (ESI) [M+H]⁺=502.4.

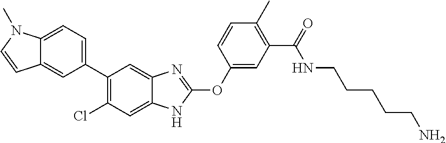 [SMILES: Cc1ccc(Oc2nc3cc(-c4ccc5c(ccn5C)c4)c(Cl)cc3[nH]2)cc1C(=O)NCCCCCN]

Linker 31: N-(5-aminopentyl)-5-((6-chloro-5-(1-methyl-1H-indol-5-yl)-1H-benzo[d]imidazol-2-yl)oxy)-2-methylbenzamide (XH168-167). Yellow solid, 64 mg, 72% yield. MS (ESI) [M+H]⁺=516.9.

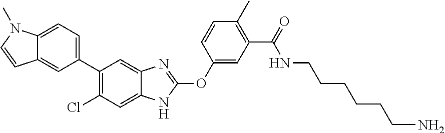 [SMILES: Cc1ccc(Oc2nc3cc(-c4ccc5c(ccn5C)c4)c(Cl)cc3[nH]2)cc1C(=O)NCCCCCCN]

Linker 32: N-(6-aminohexyl)-5-((6-chloro-5-(1-methyl-1H-indol-5-yl)-1H-benzo[d]imidazol-2-yl)oxy)-2-methylbenzamide (XH168-168). Yellow solid, 72 mg, 79% yield. MS (ESI) [M+H]⁺=530.1.

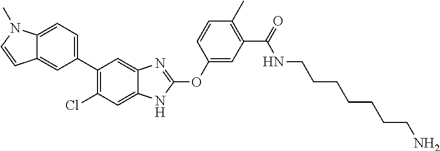 [SMILES: Cc1ccc(Oc2nc3cc(-c4ccc5c(ccn5C)c4)c(Cl)cc3[nH]2)cc1C(=O)NCCCCCCCN]

Linker 33: N-(7-aminoheptyl)-5-((6-chloro-5-(1-methyl-1H-indol-5-yl)-1H-benzo[d]imidazol-2-yl)oxy)-2-methylbenzamide (XH168-169). Yellow solid, 73 mg, 79% yield. MS (ESI) [M+H]⁺=544.5.

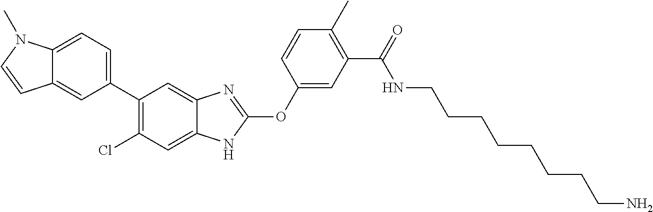 [SMILES: Cc1ccc(Oc2nc3cc(-c4ccc5c(ccn5C)c4)c(Cl)cc3[nH]2)cc1C(=O)NCCCCCCCCN]

Linker 34: N-(8-aminooctyl)-5-((6-chloro-5-(1-methyl-1H-indol-5-yl)-1H-benzo[d1]imidazol-2-yl)oxy)-2-methylbenzamide (XH168-170). Yellow solid, 69 mg, 73% yield. MS (ESI) [M+H]⁺=558.4

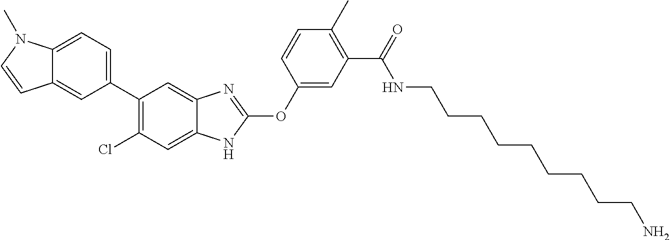 [SMILES: Cc1ccc(Oc2nc3cc(-c4ccc5c(ccn5C)c4)c(Cl)cc3[nH]2)cc1C(=O)NCCCCCCCCCN]

Linker 35: N-(9-aminononyl)-5-((6-chloro-5-(1-methyl-1H-indol-5-yl)-1H-benzo[d1]imidazol-2-yl)oxy)-2-methylbenzamide (XH168-171). Yellow solid, 78 mg, 81% yield. MS (ESI) [M+H]⁺=572.4.

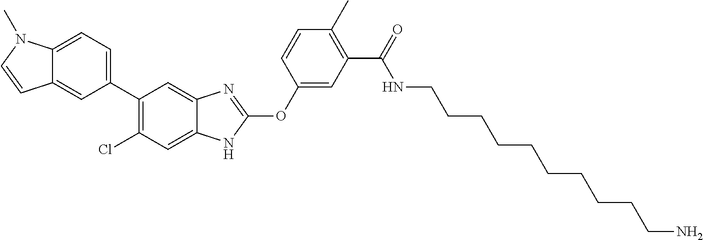 [SMILES: Cc1ccc(Oc2nc3cc(-c4ccc5c(ccn5C)c4)c(Cl)cc3[nH]2)cc1C(=O)NCCCCCCCCCCN]

Linker 36: N-(10-aminodecyl)-5-((6-chloro-5-(1-methyl-1H-indol-5-yl)-1H-benzo[d]imidazol-2-yl)oxy)-2-methylbenzamide (XH168-172) Yellow solid, 75 mg, 87% yield. MS (ESI) [M+H]⁺=586.5.

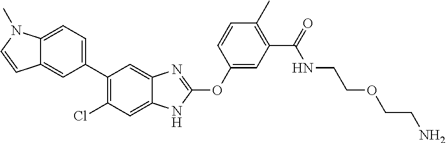 [SMILES: Cc1ccc(Oc2nc3cc(-c4ccc5c(ccn5C)c4)c(Cl)cc3[nH]2)cc1C(=O)NCCOCCN]

Linker 37: N-(2-(2-aminoethoxy)ethyl)-5-((6-chloro-5-(1-methyl-1H-indol-5-yl)-1H-benzo[d]imidazol-2-yl)oxy)-2-methylbenzamide (XH168-173). Yellow solid, 65 mg, 73% yield. MS (ESI)[M+H]⁺=518.1

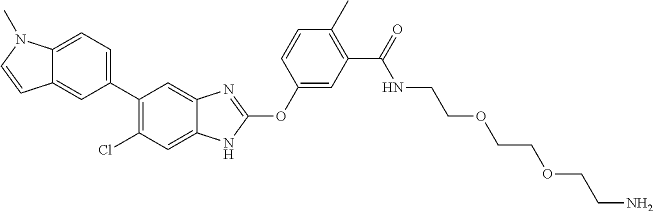 [SMILES: Cc1ccc(Oc2nc3cc(-c4ccc5c(ccn5C)c4)c(Cl)cc3[nH]2)cc1C(=O)NCCOCCOCCN]

Linker 38: N-(2-(2-(2-aminoethoxy)ethoxy)ethyl)-5-((6-chloro-5-(1-methyl-1H-indol-5-yl)-1H-benzo[d]imidazol-2-yl)oxy)-2-methylbenzamide (XH168-174). Yellow solid, 79 mg, 81% yield. MS (ESI) [M+H]⁺=562.5.

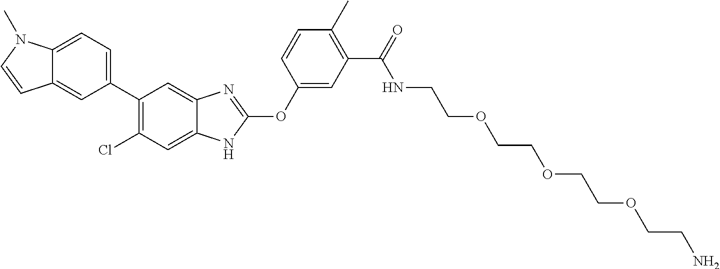 [SMILES: Cc1ccc(Oc2nc3cc(-c4ccc5c(ccn5C)c4)c(Cl)cc3[nH]2)cc1C(=O)NCCOCCOCCOCCN]

Linker 39: N-(2-(2-(2-(2-aminoethoxy)ethoxy)ethoxy)ethyl)-5-((6-chloro-5-(1-methyl-1H-indol-5-yl)-1H-benzo[d]imidazol-2-yl)oxy)-2-methylbenzamide (XH168-175). Yellow solid, 76 mg, 76% yield. MS (ESI) [M+H]⁺=606.3.

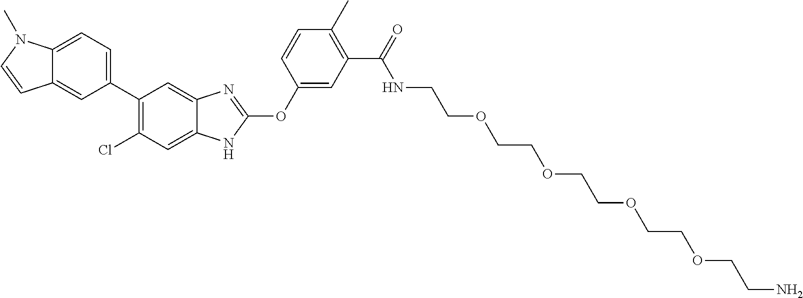 [SMILES: Cc1ccc(Oc2nc3cc(-c4ccc5c(ccn5C)c4)c(Cl)cc3[nH]2)cc1C(=O)NCCOCCOCCOCCOCCN]

Linker 40: N-(14-amino-3,6,9,12-tetraoxatetradecyl)-5-((6-chloro-5-(1-methyl-1H-indol-5-yl)-1H-benzo[d]imidazol-2-yl)oxy)-2-methylbenzamide (XH168-176). Yellow solid, 86 mg, 81% yield. MS (ESI) [M+H]⁺=650.2.

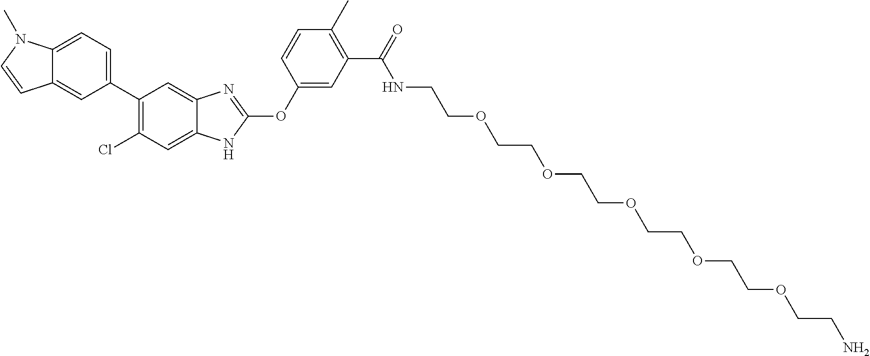 [SMILES: Cc1ccc(Oc2nc3cc(-c4ccc5c(ccn5C)c4)c(Cl)cc3[nH]2)cc1C(=O)NCCOCCOCCOCCOCCOCCN]

Linker 41: N-(17-amino-3,6,9,12,15-pentaoxaheptadecyl)-5-((6-chloro-5-(1-methyl-1H-indol-5-yl)-1H-benzo[d]imidazol-2-yl)oxy)-2-methylbenzamide (XH168-177). Yellow solid, 89 mg, 81% yield. MS (ESI) [M+H]⁻=694.5.

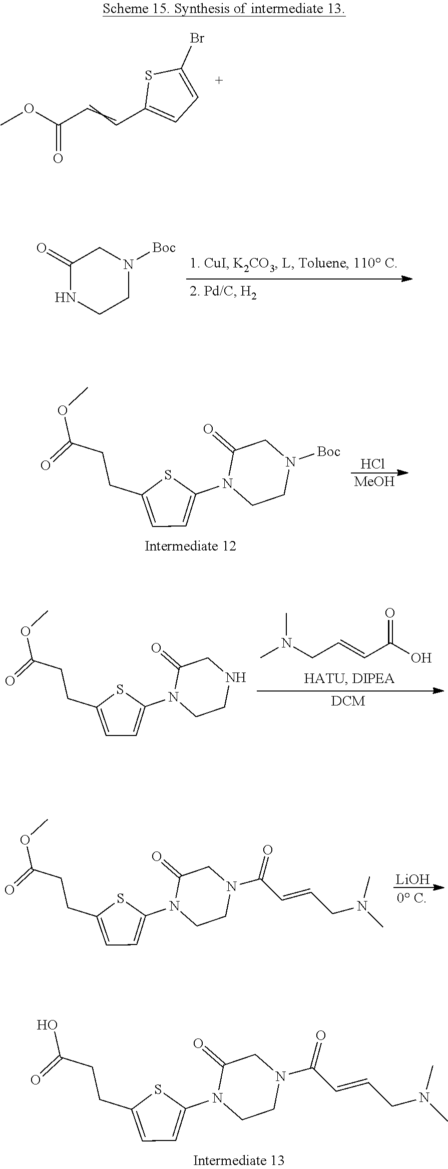

(E)-3-(5-(4-(4-(dimethylamino)but-2-enoyl)-2-oxopiperazin-1-yl)thiophen-2-yl)propanoic acid (Intermediate 13) methyl 3-(5-bromothiophen-2-yl)acrylate (246 mg, 1.0 mmol, 1.0 eq), tert-butyl 3-oxopiperazine-1-carboxylate (300 mg, 1.5 mmol, 1.5 eq), CuI (85 mg, 0.5 mmol), N¹,N²-dimethylethane-1,2-diamine (88 mg, 1.0 mmol, 1.0 eq), K₂CO₃ (276 mg, 2.0 mmol, 2.0 eq) were added Toluene (5 mL). The mixture was stirred at 110° C. under N₂ for 6 h. The mixture was purified by ISCO. The obtained product was then dissolved in MeOH (5 mL) followed by Pd/C (60 mg) ed. The mixture was stirred at room temperature under 1 atm H₂ for 1 h then filtered to yield intermediate 12 as a white solid (200 mg, 55%). ¹H NMR (400 MHz, CDCl₃) δ 6.62 (s, 1H), 6.48 (s, 1H), 4.28 (s, 2H), 3.81 (s, 4H), 3.68 (s, 3H), 3.08 (t, J=7.7 Hz, 2H), 2.68 (d, J=7.7 Hz, 2H), 1.48 (s, 9H). MS (ESI) [M+H]⁺=369.2.

Intermediate 13 (184 mg, 0.5 mmol, 1.0 eq) was dissolved in MeOH (2 mL) and HCl (4M in dioxane, 2 mL, 8 mmol), the mixture was stirred at room temperature for 3 h, then removed all the volatiles. The resulted residue (71 mg, 0.55 mmol, 1.1 eq), HATU (285 mg, 0.75 mmol, 1.5 eq), DIPEA (194 mg, 1.5 mmol, 3.0 eq) were stirred in DCM (5 mL) at room temperature for 30 min. Then removed all the volatiles, and the mixture was purified via reverse-ISCO. The purified compound (120 mg, 0.33 mmol, 1.0 eq) was dissolved in MeOH (2 mL). LiOH aq. (2M, 0.5 mL, 1 mmol) was added dropwise at 0° C. The mixture was stirred at 0° C. for 20 min, then was purified via reverse-ISCO to yield title compound as a brown solid (100 mg, 83%).¹H NMR (400 MHz, CD₃OD) δ 7.08-6.64 (m, 4H), 4.13-3.89 (m, 6H), 3.15-3.04 (m, 2H), 2.98-2.87 (m, 8H), 2.73-2.62 (m, 2H). MS (ESI) [M+H]⁺=366.3.

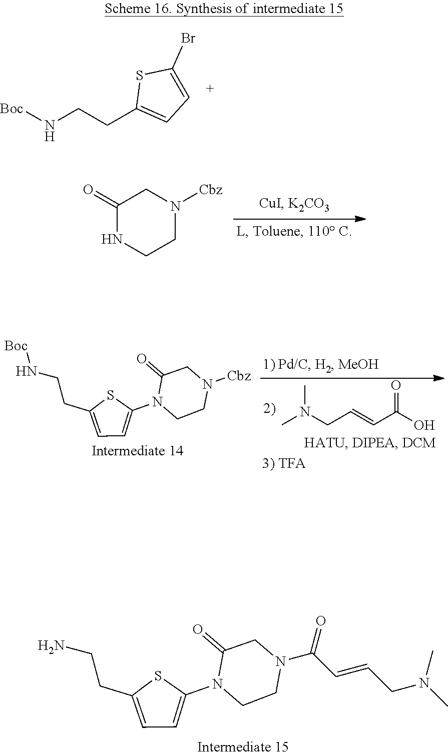

(E)-1-(5-(2-aminoethyl)thiophen-2-yl)-4-(4-(dimethylamino)but-2-enoyl)piperazin-2-one (Intermediate 15) tert-butyl (2-(5-bromothiophen-2-yl)ethyl)carbamate (306 mg, 1.0 mmol, 1.0 eq), benzyl 3-oxopiperazine-1-carboxylate (350 mg, 1.5 mmol, 1.5 eq), CuI (85 mg, 0.5 mmol), N¹,N²-dimethylethane-1,2-diamine (88 mg, 1.0 mmol, 1.0 eq), K₂CO₃ (276 mg, 2.0 mmol, 2.0 eq) were added Toluene (5 mL). The mixture was stirred at 110° C. under N₂ for 6 h. The mixture was purified ISCO to yield intermediate 14 as a white solid (278 mg, 61%). ¹H NMR (400 MHz, CD₃OD) δ 7.47-7.32 (m, 5H), 6.69 (s, 2H), 5.20 (s, 2H), 4.33 (s, 2H), 3.91 (s, 4H), 3.33-3.25 (m, 2H), 2.94-2.87 (m, 2H), 1.45 (s, 9H). MS (ESI) [M+H]⁺=460.4.

Intermediate 14 (459 mg, 1.0 mmol, 1.0 eq), Pd/C (120 mg), Pd(OH)₂ (120 mg) were added to MeOH (5 mL), the mixture was stirred under 10 atm H₂ at 60° C. for 2 h. The mixture was filtered and the product was used without further purification. MS (ESI) [M+H]⁺=326.3. The obtained crude product (160 mg, 0.5 mmol, 1.0 eq), (E)-4-(dimethylamino)but-2-enoic acid (71 mg, 0.55 mmol, 1.1 eq), HATU (285 mg, 0.75 mmol, 1.5 eq), DIPEA (194 mg, 1.5 mmol, 3.0 eq) were stirred in DCM (5 mL) at room temperature for 30 min. Then removed all the volatiles, and the mixture was purified via reverse-ISCO. The obtained product was stirred with DCM/TFA (1 mL/1 mL) for 20 min. Then removed all the volatiles, and the mixture was purified via reverse-ISCO to yield title compound as a white solid (150 mg, 98%). ¹H NMR (400 MHz, CD₃OD) δ 7.17-6.74 (m, 4H), 4.19-3.93 (m, 6H), 3.26-3.19 (m, 2H), 3.18-3.11 (m, 2H), 3.01-2.90 (m, 8H). MS (ESI) [M+H]⁺=337.2.

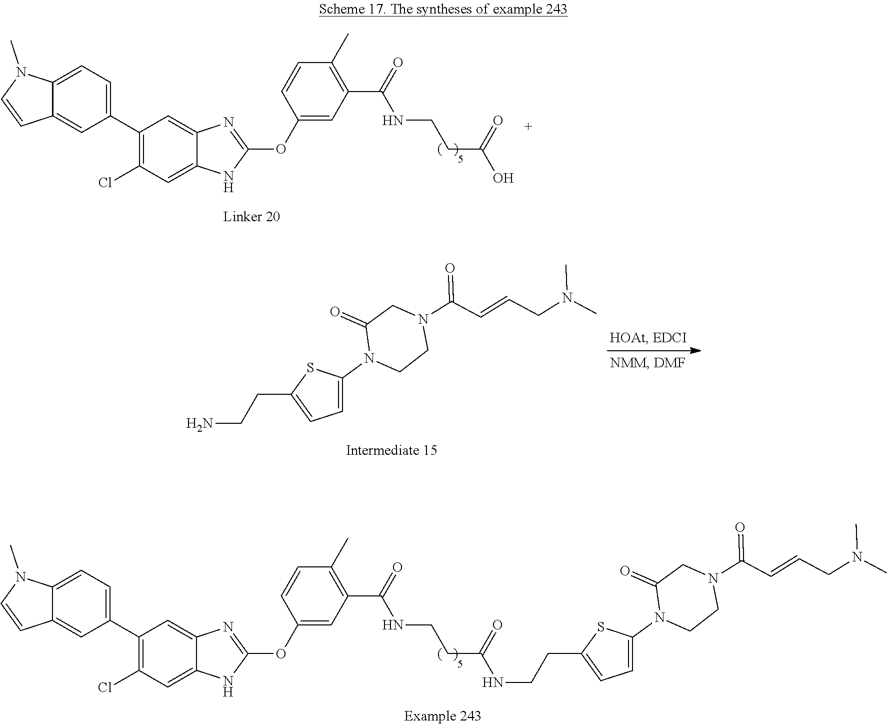

Example 243: (E)-5-((6-chloro-5-(1-methyl-1H-indol-5-yl)-1H-benzo[d]imidazol-2-yl)oxy)-N-(7-((2-(5-(4-(4-(dimethylamino)but-2-enoyl)-2-oxopiperazin-1-yl)thiophen-2-yl)ethyl)amino)-7-oxoheptyl)-2-methylbenzamide (QC179-047) Linker 20 (12 mg, 0.025 mmol, 1.0 eq), intermediate 15 (10 mg, 0.028 mmol, 1.1 eq), EDC HCl (7 mg, 0.038 mmol, 1.5 eq), HOAt (5 mg, 0.038 mmol, 1.5 eq), NMM (8 mg, 0.075 mmol, 3.0 eq) were stirred in 2 mL DMF at room temperature for 2 h. Then the mixture was purified via Prep-HPLC to yield titled compound as a white solid (8 mg, 46% yield). ¹H NMR (400 MHz, CD₃OD) δ 7.58 (s, 1H), 7.51 (s, 1H), 7.46-7.32 (m, 5H), 7.27-7.21 (m, 2H), 6.94 (dd, J=32.4, 15.1 Hz, 1H), 6.77 (br, 1H), 6.70-6.64 (m, 2H), 6.49 (s, 1H), 4.50 (s, 1H), 4.43 (s, 1H), 4.04 (s, 2H), 3.95 (s, 3H), 3.87 (s, 4H), 3.44-3.36 (m, 4H), 2.92 (s, 8H), 2.46 (s, 3H), 2.24-2.13 (m, 2H), 1.64 (s, 4H), 1.47-1.30 (m, 4H). MS (ESI) [M+H]⁺=877.5.

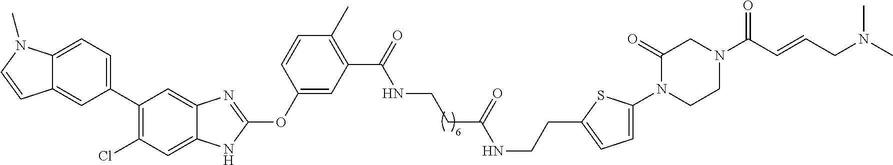

Example 244: (E)-5-((6-chloro-5-(1-methyl-1H-indol-5-yl)-1H-benzo[d]imidazol-2-yl)oxy)-N-(8-((2-(5-(4-(4-(dimethylamino)but-2-enoyl)-2-oxopiperazin-1-yl)thiophen-2-yl)ethyl)amino)-8-oxooctyl)-2-methylbenzamide (QC179-048) Example 244 was synthesized following the same procedure for preparing example 243 from linker 21. White solid, 9 mg, 50% yield. ¹H NMR (400 MHz, CD₃OD) δ 7.67-7.11 (m, 10H), 7.02-6.85 (m, 1H), 6.78 (br, 1H), 6.68-6.56 (m, 2H), 4.52-4.39 (m, 2H), 4.13-3.86 (m, 9H), 3.40 (s, 4H), 2.91 (s, 8H), 2.45 (s, 3H), 2.15 (s, 2H), 1.61 (s, 4H), 1.46-1.30 (s, 6H). MS (ESI) [M+H]⁺=891.5.

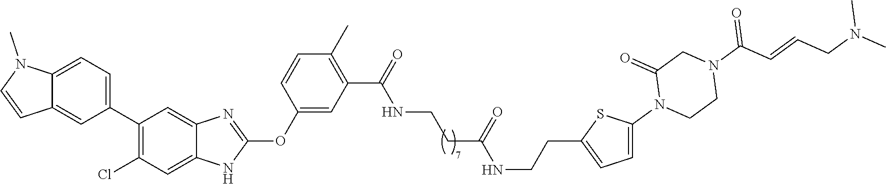

Example 245: (E)-5-((6-chloro-5-(1-methyl-1H-indol-5-yl)-1H-benzo[d]imidazol-2-yl)oxy)-N-(9-((2-(5-(4-(4-(dimethylamino)but-2-enoyl)-2-oxopiperazin-1-yl)thiophen-2-yl)ethyl)amino)-9-oxononyl)-2-methylbenzamide (QC179-049) Example 245 was synthesized following the same procedure for preparing example 243 from linker 22. White solid, 9 mg, 45% yield. ¹H NMR (400 MHz, CD₃OD) δ 7.58 (s, 1H), 7.51 (s, 1H), 7.44-7.36 (m, 5H), 7.29-7.20 (m, 2H), 6.95 (dd, J=33.6, 15.2 Hz, 1H), 6.77 (br, 1H), 6.67 (s, 2H), 6.49 (s, 1H), 4.50 (s, 1H), 4.44 (s, 1H), 4.04 (s, 2H), 3.98-3.92 (m, 3H), 3.91-3.95 (m, 4H), 3.38 (s, 4H), 2.92 (s, 8H), 2.46 (s, 3H), 2.20-2.11 (m, 2H), 1.68-1.54 (m, 4H), 1.48-1.24 (m, 8H). MS (ESI) [M+H]⁺=905.6.

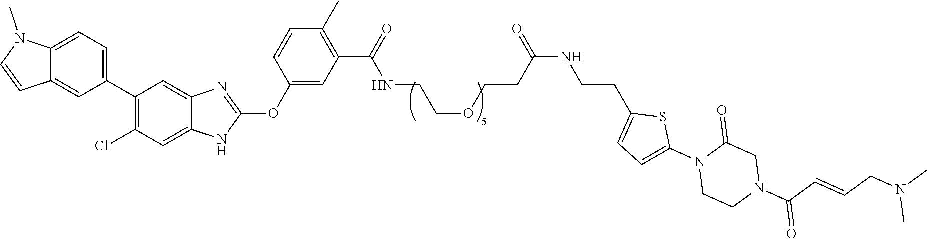

Example 246: (E)-1-(5-((6-chloro-5-(1-methyl-1H-indol-5-yl)-1H-benzo[d]imidazol-2-yl)oxy)-2-methylbenzamido)-N-(2-(5-(4-(4-(dimethylamino)but-2-enoyl)-2-oxopiperazin-1-yl)thiophen-2-yl)ethyl)-3,6,9,12,15-pentaoxaoctadecan-18-amide (QC179-050) Example 246was synthesized following the same procedure for preparing example 243 from linker 27. White solid, 8 mg, 40% yield. ¹H NMR (400 MHz, CD₃OD) δ 7.58 (s, 1H), 7.52 (s, 1H), 7.45-7.37 (m, 5H), 7.27-7.21 (m, 2H), 7.00-6.87 (m, 1H), 6.77 (s, 1H), 6.69 (s, 1H), 6.65 (s, 1H), 6.49 (s, 1H), 4.49 (s, 1H), 4.43 (s, 1H), 4.03 (s, 2H), 3.98-3.93 (m, 3H), 3.87 (s, 4H), 3.66-3.50 (m, 26H), 2.92 (s, 8H), 2.47 (s, 3H). MS (ESI) [M+H]⁺=1141.6.

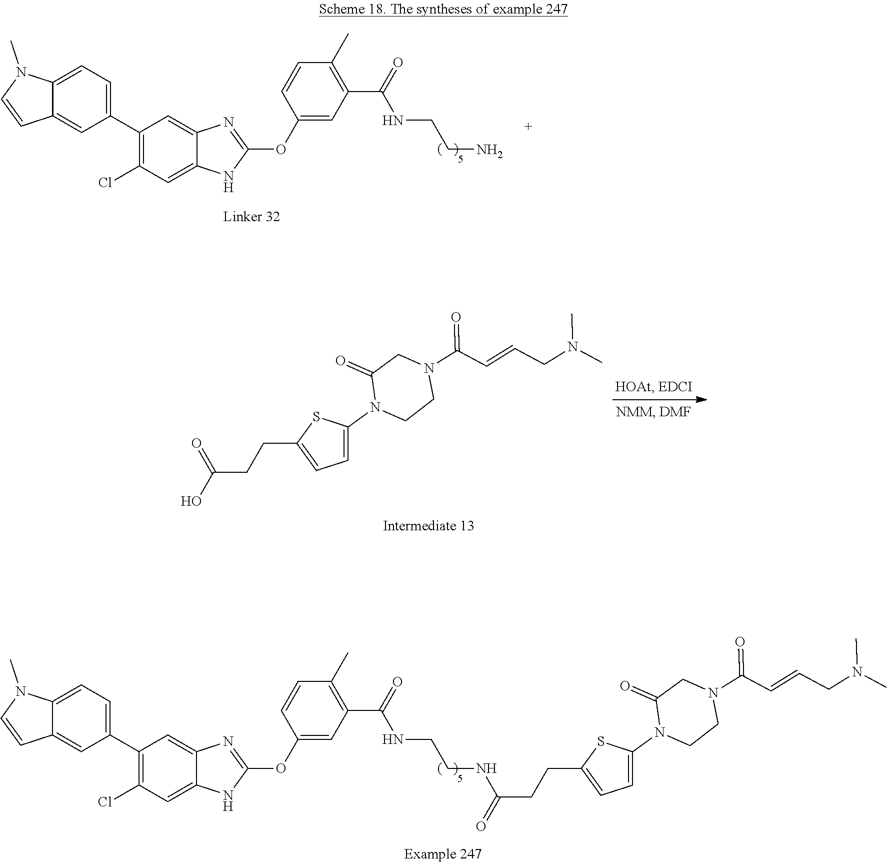

Example 247: (L)-N-(2-((5-((6-chloro-5-(1-methyl-1H-indol-5-yl)-1H-benzo[d]imidazol-2-yl)oxy)-2-methylbenzoyl)tetramethyl-1⁷-azaneyl)ethyl)-3-(5-(4-(4-(dimethylamino)but-2-enoyl)-2-oxopiperazin-1-yl)thiophen-2-yl)propenamide (QC179-051) Linker 32 (14 mg, 0.020 mmol, 1.0 eq), intermediate 13 (11 mg, 0.022 mmol, 1.1 eq), EDC HCl (6 mg, 0.030 mmol, 1.5 eq), HOAt (4 mg, 0.030 mmol, 1.5 eq), NMM (6 mg, 0.060 mmol, 3.0 eq) were stirred in 1 mL DMF at room temperature for 2 h. Then the mixture was purified via Prep-HPLC to yield titled compound as a white solid (8 mg, 40% yield). ¹H NMR (400 MHz, CD₃OD) δ 7.57 (s, 1H), 7.51 (s, 1H), 7.45-7.36 (m, 5H), 7.27-7.21 (m, 2H), 6.93 (dd, J=31.9, 14.9 Hz, 1H), 6.76 (s, 1H), 6.63 (s, 2H), 6.49 (s, 1H), 4.49 (s, 1H), 4.43 (s, 1H), 4.03 (s, 2H), 3.99-3.93 (m, 3H), 3.87 (s, 4H), 3.15 (s, 2H), 3.08-3.00 (m, 2H), 2.92 (s, 6H), 2.54-2.44 (m, 5H), 1.61 (s, 2H), 1.53-1.28 (m, 8H). MS (ESI) [M+H]⁺=877.4.

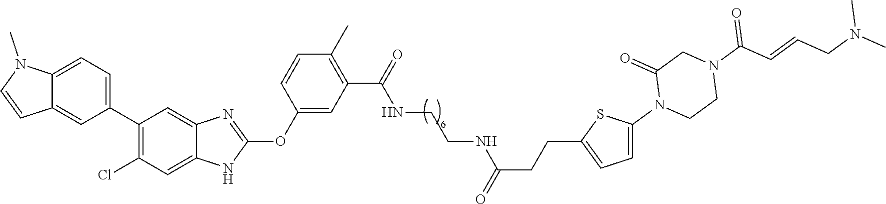

Example 248: (E)-N-(2-((5-((6-chloro-5-(1-methyl-1H-indol-5-yl)-1H-benzo[d]imidazol-2-yl)oxy)-2-methylbenzoyl)pentamethyl-I⁸-azaneyl)ethyl)-3-(5-(4-(4-(dimethylamino)but-2-enoyl)-2-oxopiperazin-1-yl)thiophen-2-yl)propenamide (QC179-052) Example 248 was synthesized following the same procedure for preparing example 247 from linker 33. White solid 7 mg, 43% yield. ¹H NMR (400 MHz, CD₃OD) δ 7.58 (s, 1H), 7.51 (s, 1H), 7.43-7.36 (m, 5H), 7.27-7.21 (m, 2H), 7.01-7.88 (m, 1H), 6.76 (s, 1H), 6.64 (s, 2H), 6.49 (s, 1H), 4.51-4.37 (m, 2H), 4.08-4.02 (m, 2H), 3.98-3.93 (m, 3H), 3.87 (s, 4H), 3.18-3.11 (m, 2H), 3.07-3.01 (m, 2H), 2.92 (s, 6H), 2.54-2.43 (m, 5H), 1.62-1.32 (m, 12H). MS (ESI) [M+H]⁺=891.4.

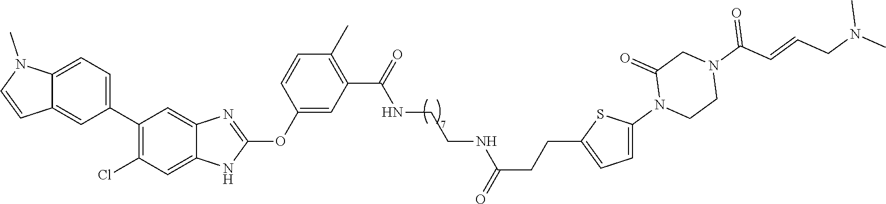

Example 249: (E)-N-(2-((5-((6-chloro-5-(1-methyl-1H-indol-5-yl)-1H-benzo[d]imidazol-2-yl)oxy)-2-methylbenzoyl)hexamethyl-I⁹-azaneyl)ethyl)-3-(5-(4-(4-(dimethylamino)but-2-enoyl)-2-oxopiperazin-1-yl)thiophen-2-yl)propenamide (QC179-053) Example 249 was synthesized following the same procedure for preparing example 247 from linker 34. White solid, 10 mg, 48% yield. ¹H NMR (400 MHz, CD₃OD) δ 7.58 (s, 1H), 7.51 (s, 1H), 7.44-7.35 (m, 5H), 7.27-7.21 (m, 2H), 6.95 (dd, J=35.2, 15.7 Hz, 1H), 6.77 (s, 1H), 6.67-6.47 (m, 3H), 4.51-4.38 (m, 2H), 4.05-3.85 (m, 9H), 3.17-3.05 (m, 4H), 2.92 (s, 6H), 2.52-2.44 (m, 5H), 1.62 (s, 2H), 1.43-1.23 (m, 12H). MS (ESI) [M+H]⁺==905.4.

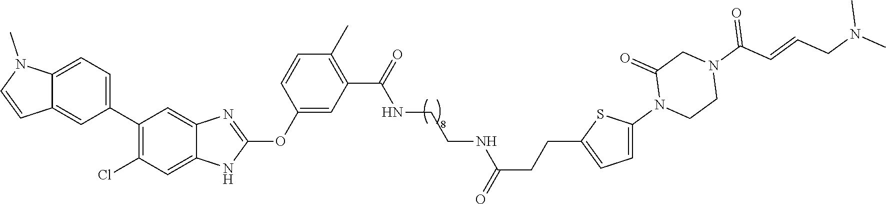

Example 250: (E)-N-(2-((5-((6-chloro-5-(1-methyl-1H-indol-5-yl)-1H-benzo[d]imidazol-2-yl)oxy)-2-methylbenzoyl)heptamethyl-I¹⁰-azaneyl)ethyl)-3-(5-(4-(4-(dimethylamino)but-2-enoyl)-2-oxopiperazin-1-yl)thiophen-2-yl)propenamide (QC179-054) Example 250 was synthesized following the same procedure for preparing example 247 from linker 35. White solid 9 mg, 50% yield. ¹H NMR (400 MHz, CD₃OD) δ 7.58-7.14 (m, 10H), 7.02-6.88 (m, 1H), 6.77 (s, 1H), 6.66-6.49 (m, 2H), 4.41 (s, 2H), 4.03-3.71 (m, 9H), 3.13-2.99 (m, 4H), 2.91 (s, 6H), 2.53-2.44 (m, 5H), 1.60 (s, 2H), 1.41-1.23 (m, 14H). MS (ESI) [M+H]⁺=919.3.

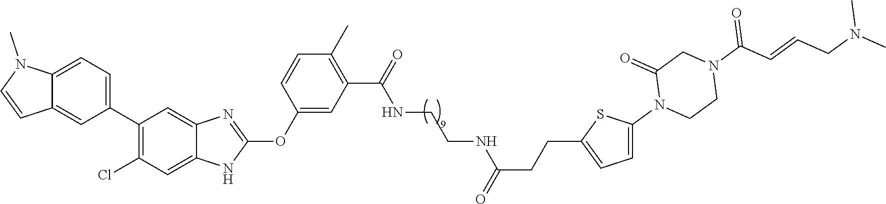

Example 251: (E)-N-(2-((5-((6-chloro-5-(1-methyl-H-indol-5-yl)-1H-benzo[d]imidazol-2-yl)oxy)-2-methylbenzoyl)octamethyl-I¹¹-azaneyl)ethyl)-3-(5-(4-(4-(dimethylamino)but-2-enoyl)-2-oxopiperazin-1-yl)thiophen-2-yl)propenamide (QC179-055) Example 251 was synthesized following the same procedure for preparing example 247 from linker 36. White solid 7 mg, 40% yield. ¹H NMR (400 MHz, CD₃OD) δ 7.58 (s, 1H), 7.52 (s, 1H), 7.45-7.34 (m, 5H), 7.28-7.21 (m, 2H), 6.94 (dd, J=34.8, 15.2 Hz, 1H), 6.78 (s, 1H), 6.67-6.60 (m, 2H), 6.49 (s, 1H), 4.49 (s, 1H), 4.43 (s, 1H), 4.04 (s, 2H), 3.99-3.85 (m, 7H), 3.16-3.03 (m, 4H), 2.92 (s, 6H), 2.55-2.42 (m, 5H), 1.64 (s, 2H), 1.47-1.22 (m, 16H). MS (ESI) [M+H]⁺=933.5.

Procedures for the Synthesis of cGAS Based Bivalent Compounds

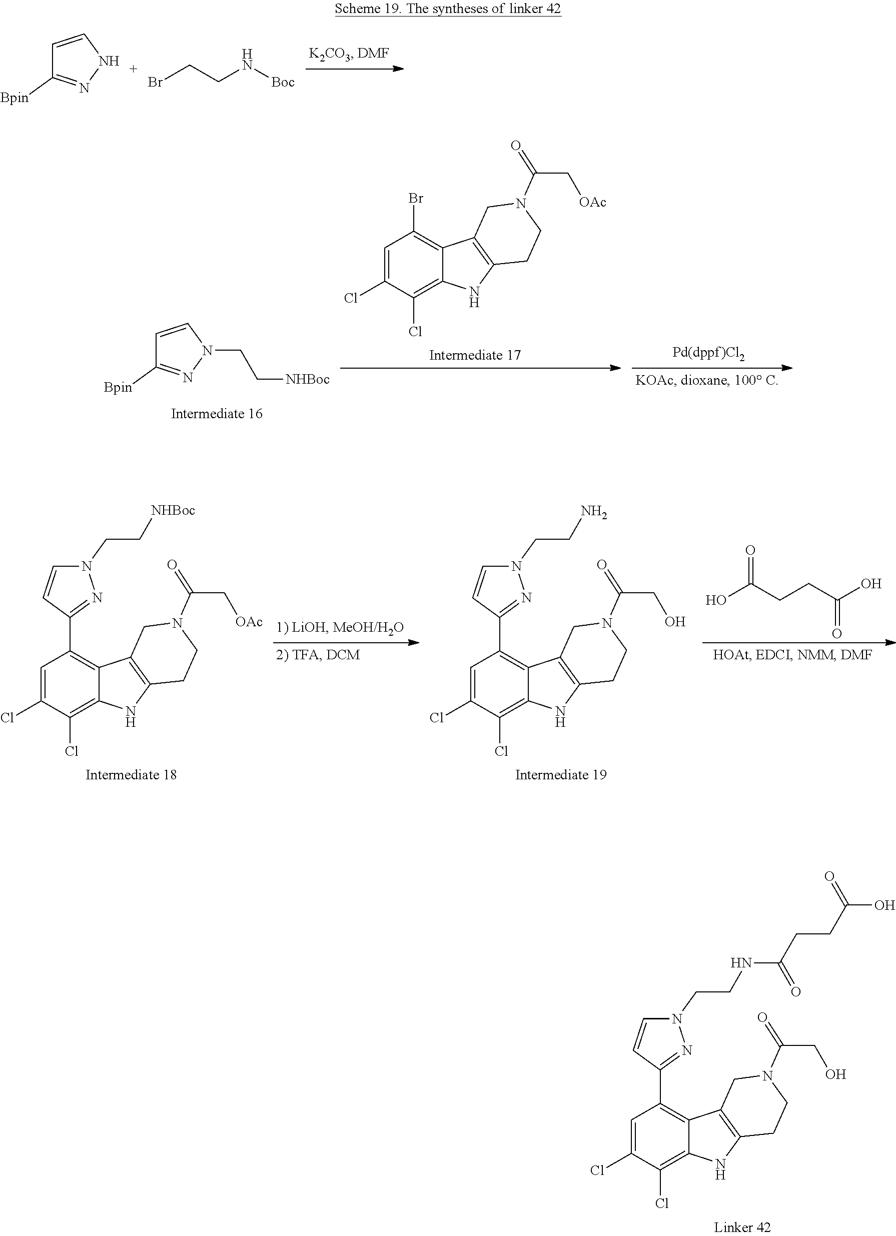

Linker 42: 2-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)acetamide To a solution of 3-(4,4,5,5-tetramethyl-1,3,2-dioxaborolan-2-yl)-1H-pyrazole (7.3 g, 37.6 mmol, 1.0 eq) in MeCN (30 ml) was added K₂CO₃ (15.6 g, 112.9 mmol, 3.0 eq) and tert-butyl (2-bromoethyl)carbamate (10.1 g, 45.1 mmol, 1.2 eq). The mixture was stirred at 80° C. for 6 h. After that, water was added and the mixture was extracted with ethyl acetate. The organic layer was collected, and excess solvent was removed to get intermediate 16 and used for the next step directly without further purification.

To a solution of intermediate 17 (1.05 g, 2.5 mmol, 1 eq) (prepared following previous reported lit.) (Lama et al., 2019) and intermediate 16 (3.2 g, 9.5 mmol, 3.8 eq) in dioxane (40 ml) were added KOAc (981 mg, 10.0 mmol, 4.0 eq) and Pd(dppf)Cl₂(408 mg, 0.5 mmol, 0.2 eq). The mixture was stirred at 100° C. for 6 h under N₂ followed concentrated under reduced pressure. The resulted residue was purified by column chromatography (SiO₂, DCM/MeOH=10:1) to give intermediate 18 as a brown oil (894 mg, 65% yield). ¹H NMR (400 MHz, DMSO-d₆) δ 7.88 (s, 1H), 7.32 (d, J=9.7 Hz, 1H), 6.70 (dd, J=27.9, 2.3 Hz, 1H), 4.82 (d, J=61.8 Hz, 2H), 4.68-4.36 (m, 4H), 3.76 (dt, J=45.2, 5.9 Hz, 2H), 2.89 (dt, J=46.7, 5.8 Hz, 2H), 2.07 (d, J=1.9 Hz, 5H), 1.11 (s, 9H).

To a solution of intermediate 18 (420 mg, 0.76 mmol, 1 eq) in MeOH/H₂O (10:1, 4.4 ml) was added LiOH·H₂O (91 mg, 2.3 mmol, 3 eq). The mixture was stirred at rt for 10 min followed by concentrated and diluted with H₂O. After that, the pH value of the solution was adjusted to 6 with HCl aq. solution (1M) followed by concentration. The residue was re-dissolved in DCM/TFA (2:1, 7.5 mL). After stirring at rt for 1 h, excess solvent was removed and the residue was purified by prep-HPLC to give intermediate 19 as a yellow solid, (178.6 mg, 45% yield). ¹H NMR (400 MHz, CD₃OD) δ 7.77 (d, J=13.4 Hz, 1H), 7.23 (dd, J=9.9, 3.0 Hz, 1H), 6.56 (d, J=9.3 Hz, 1H), 4.73-4.47 (m, 4H), 4.40-4.13 (m, 2H), 3.97-3.46 (m, 4H), 3.07-2.81 (m, 2H).

To a solution of succinic acid (35.4 mg, 0.3 mmol, 2.0 eq) in DMF (0.75 mL) and DCM (18 mL) was added NMM (75.9 mg, 0.75 mmol, 5.0 eq), intermediate 18 (78.3 mg, 0.15 mmol, 1.0 eq), HOAt (24.5 mg, 0.18 mmol, 1.2 eq), and EDCI (34.5 mg, 0.18 mmol, 1.2eq) at 0° C. The resulted reaction solution was stirred at 0° C. for 6 h before being stirred at room temperature overnight followed by concentrated. The resulted residue was purified by reverse-phase chromatography to yield the titled product (38.1 mg, 50% yield). ¹H NMR (400 MHz, CD₃OD) δ 7.78-7.65 (m, 1H), 7.21 (d, J=18.1 Hz, 1H), 6.49 (dd, J=23.8, 2.3 Hz, 1H), 4.55 (d, J=7.0 Hz, 2H), 4.42-4.16 (m, 4H), 3.99-3.61 (m, 4H), 2.94 (dt, J=22.2, 5.8 Hz, 2H), 2.59 (t, J=6.8 Hz, 2H), 2.46 (dt, J=10.8, 6.7 Hz, 2H).

Linker 43-55 were synthesized following the same procedure for preparing linker 42.

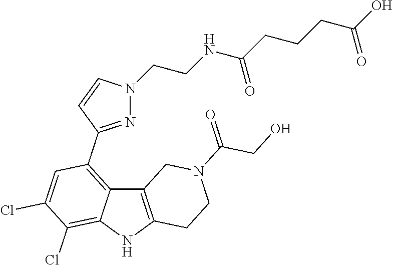 [SMILES: O=C(O)CCCC(=O)NCCn1ccc(-c2cc(Cl)c(Cl)c3[nH]c4c(c23)CN(C(=O)CO)CC4)n1]

Linker 43: 5-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-5-oxopentanoic acid. Brown solid, yield 45%. 1H NMR (400 MHz, CD₃OD) δ 7.67 (dd, J=16.3, 2.3 Hz, 1H), 7.21 (d, J=19.1 Hz, 1H), 6.48 (dd, J=26.0, 2.3 Hz, 1H), 4.68-4.14 (m, 6H), 3.99-3.63 (m, 4H), 3.06-2.84 (m, 2H), 2.42-2.13 (m, 4H), 1.85 (d, J=29.4 Hz, 2H).

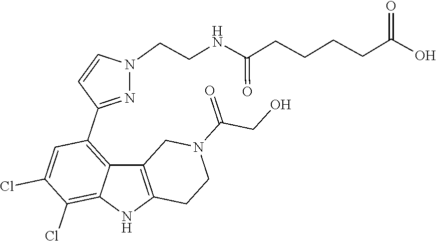 [SMILES: O=C(O)CCCCC(=O)NCCn1ccc(-c2cc(Cl)c(Cl)c3[nH]c4c(c23)CN(C(=O)CO)CC4)n1]

Linker 44: 6-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-6-oxohexanoic acid. Brown solid, yield 47%. 1H NMR (400 MHz, CD₃OD) δ 7.69 (dd, J=17.0, 2.2 Hz, 1H), 7.23 (d, J=18.8 Hz, 1H), 6.51 (dd, J=26.7, 2.3 Hz, 1H), 4.72-4.19 (m, 6H), 4.00-3.59 (m, 4H), 3.05-2.86 (m, 2H), 2.21 (d, J=53.8 Hz, 4H), 1.58 (d, J=63.0 Hz, 4H).

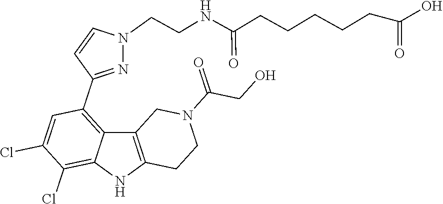 [SMILES: O=C(O)CCCCCC(=O)NCCn1ccc(-c2cc(Cl)c(Cl)c3[nH]c4c(c23)CN(C(=O)CO)CC4)n1]

Linker 45: 7-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-7-oxoheptanoic acid. Brown solid, yield 53%. 1H NMR (400 MHz, CD₃OD) δ 7.66 (dd, J=17.2, 2.3 Hz, 1H), 7.20 (d, J=18.0 Hz, 1H), 6.49 (dd, J=25.4, 2.3 Hz, 1H), 4.65-4.17 (m, 6H), 3.79-3.56 (m, 4H), 3.02-2.74 (m, 2H), 2.40-2.03 (m, 4H), 1.40 (s, 6H).

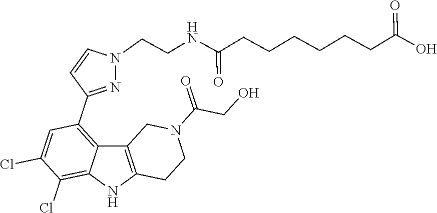 [SMILES: O=C(O)CCCCCCC(=O)NCCn1ccc(-c2cc(Cl)c(Cl)c3[nH]c4c(c23)CN(C(=O)CO)CC4)n1]

Linker 46: 8-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-8-oxooctanoic acid. Brown solid, yield 58%. ¹H NMR (400 MHz, CD₃OD) δ 7.98-7.47 (m, 1H), 7.20 (d, J=14.3 Hz, 1H), 6.50 (dd, J=23.5, 2.3 Hz, 1H), 4.70-4.18 (m, 6H), 3.99-3.88 (m, 1H), 3.78-3.57 (m, 3H), 3.01-2.74 (m, 4H), 2.34-2.05 (m, 2H), 1.73-1.13 (m, 8H).

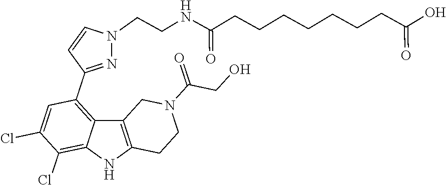 [SMILES: O=C(O)CCCCCCCC(=O)NCCn1ccc(-c2cc(Cl)c(Cl)c3[nH]c4c(c23)CN(C(=O)CO)CC4)n1]

Linker 47: 9-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-9-oxononanoic acid. Brown solid, yield 56%. ¹H NMR (400 MHz, CD₃OD) δ 7.73-7.56 (m, 1H), 7.20 (d, J=14.1 Hz, 1H), 6.49 (dd, J=24.0, 2.3 Hz, 1H), 4.68-4.19 (m, 6H), 4.00-3.61 (m, 4H), 3.06-2.76 (m, 4H), 2.35-2.06 (m, 2H), 1.73-1.03 (m, 10H).

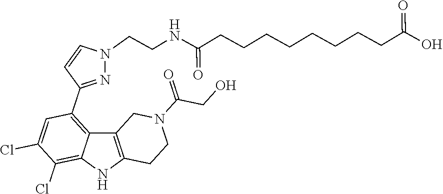 [SMILES: O=C(O)CCCCCCCCC(=O)NCCn1ccc(-c2cc(Cl)c(Cl)c3[nH]c4c(c23)CN(C(=O)CO)CC4)n1]

Linker 48: 10-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-10-oxodecanoic acid. Brown solid, yield 60%. ¹H NMR (400 MHz, CD₃OD) δ 7.65 (dd, J=20.7, 2.3 Hz, 1H), 7.20 (d, J=14.7 Hz, 1H), 6.48 (dd, J=24.8, 2.3 Hz, 1H), 4.65-4.17 (m, 6H), 3.97-3.56 (m, 4H), 2.92 (d, J=27.6 Hz, 2H), 2.18 (t, J=59.4 Hz, 4H), 1.67-1.04 (m, 12H).

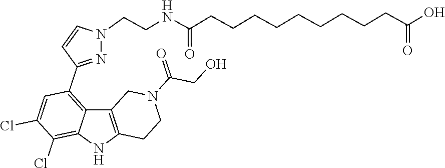 [SMILES: O=C(O)CCCCCCCCCC(=O)NCCn1ccc(-c2cc(Cl)c(Cl)c3[nH]c4c(c23)CN(C(=O)CO)CC4)n1]

Linker 49: 11-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-11-oxoundecanoic acid Brown solid, yield 47%. ¹H NMR (400 MHz, CD₃OD) δ 7.65 (dd, J=20.6, 2.3 Hz, 1H), 7.21 (d, J=13.3 Hz, 1H), 6.49 (dd, J=24.3, 2.3 Hz, 1H), 4.70-4.10 (m, 6H), 3.97-3.59 (m, 1H), 3.05-2.80 (m, 2H), 2.30-2.03 (m, 4H), 1.64-0.92 (m, 14H).

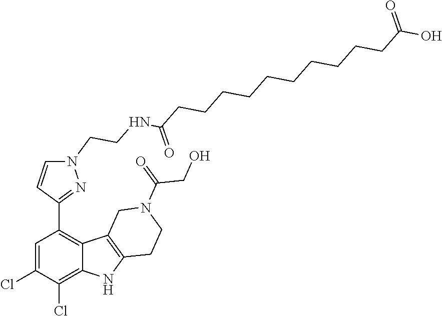 [SMILES: O=C(O)CCCCCCCCCCC(=O)NCCn1ccc(-c2cc(Cl)c(Cl)c3[nH]c4c(c23)CN(C(=O)CO)CC4)n1]

Linker 50: 12-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-12-oxododecanoic acid. Brown solid, yield 45%. ¹H NMR (400 MHz, CD₃OD) δ 7.65 (dd, J=21.4, 2.0 Hz, 1H), 7.21 (d, J=13.7 Hz, 1H), 6.48 (dd, J=25.4, 2.2 Hz, 1H), 4.70-4.18 (m, 6H), 4.00-3.56 (m, 4H), 3.09-2.81 (m, 2H), 2.38-2.02 (m, 4H), 1.64-0.93 (m, 16H).

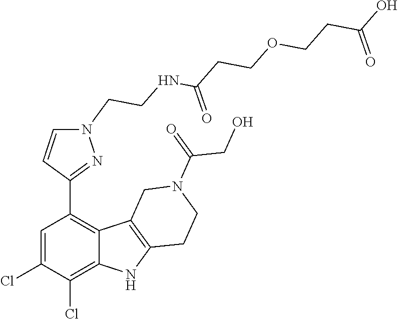 [SMILES: O=C(O)CCOCCC(=O)NCCn1ccc(-c2cc(Cl)c(Cl)c3[nH]c4c(c23)CN(C(=O)CO)CC4)n1]

Linker 51: 3-(3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropoxy)propanoic acid. Brown solid, yield 46%. ¹H NMR (400 MHz, CD₃OD) δ 7.69 (dd, J=15.3, 2.3 Hz, 1H), 7.20 (d, J=17.8 Hz, 1H), 6.49 (dd, J=23.4, 2.3 Hz, 1H), 4.62-4.17 (m, 6H), 3.97-3.56 (m, 8H), 3.02-2.82 (m, 2H), 2.57-2.31 (m, 4H).

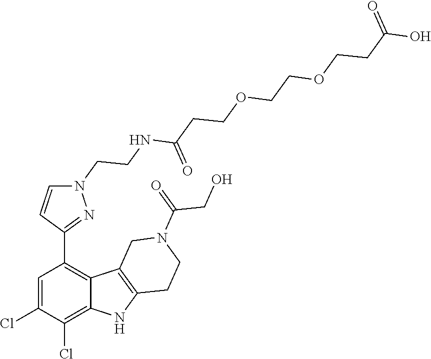 [SMILES: O=C(O)CCOCCOCCC(=O)NCCn1ccc(-c2cc(Cl)c(Cl)c3[nH]c4c(c23)CN(C(=O)CO)CC4)n1]

Linker 52: 3-(2-(3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropoxy)ethoxy)propanoic acid. Brown solid, yield 49%. ¹H NMR (400 MHz, CD₃OD) δ 7.70 (dd, J=17.0, 2.2 Hz, 1H), 7.20 (d, J=16.8 Hz, 1H), 6.48 (dd, J=24.2, 2.3 Hz, 1H), 4.62-4.15 (m, 6H), 3.97-3.42 (m, 12H), 3.01-2.82 (m, 2H), 2.54-2.32 (m, 4H).

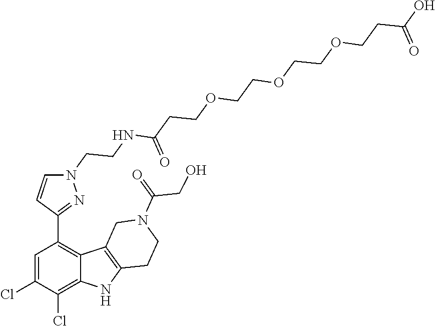 [SMILES: O=C(O)CCOCCOCCOCCC(=O)NCCn1ccc(-c2cc(Cl)c(Cl)c3[nH]c4c(c23)CN(C(=O)CO)CC4)n1]

Linker 53: 1-(3-(6,7-dichloro-2-(2-hydroxyacetyl)-2,3,4,5-tetrahydro-1H-pyrido[4,3-b]indol-9-yl)-1H-pyrazol-1-yl)-4-oxo-7,10,13-trioxa-3-azahexadecan-16-oic acid. Brown solid, yield 52%. ¹H NMR (400 MHz, CD₃OD) δ 7.81-7.62 (m, 1H), 7.30-7.15 (m, 1H), 6.57-6.40 (m, 1H), 4.64-4.19 (m, 6H), 3.98-3.45 (m, 16H), 3.04-2.84 (m, 2H), 2.58-2.31 (m, 4H).

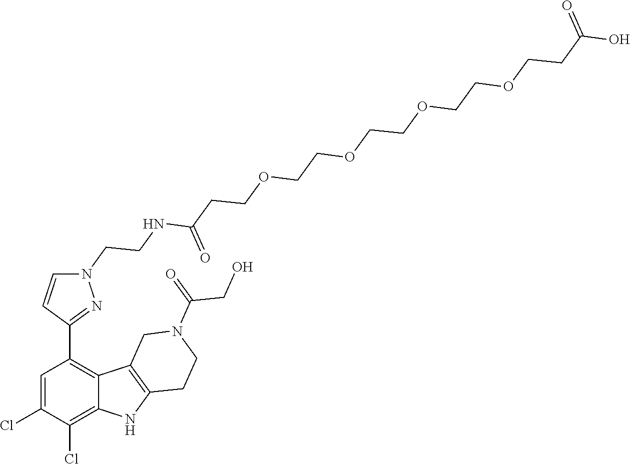 [SMILES: O=C(O)CCOCCOCCOCCOCCC(=O)NCCn1ccc(-c2cc(Cl)c(Cl)c3[nH]c4c(c23)CN(C(=O)CO)CC4)n1]

Linker 54: 1-(3-(6,7-dichloro-2-(2-hydroxyacetyl)-2,3,4,5-tetrahydro-1H-pyrido[4,3-b]indol-9-yl)-1H-pyrazol-1-yl)-4-oxo-7,10,13,16-tetraoxa-3-azanonadecan-19-oic acid. Brown solid, yield 53%. ¹H NMR (400 MHz, CD₃OD) δ 7.80-7.66 (m, 1H), 7.23 (d, J=17.3 Hz, 1H), 6.52 (dd, J=24.5, 2.3 Hz, 1H), 4.63-4.17 (m, 6H), 3.79-3.47 (m, 20H), 3.01-2.86 (m, 2H), 2.63-2.28 (m, 4H).

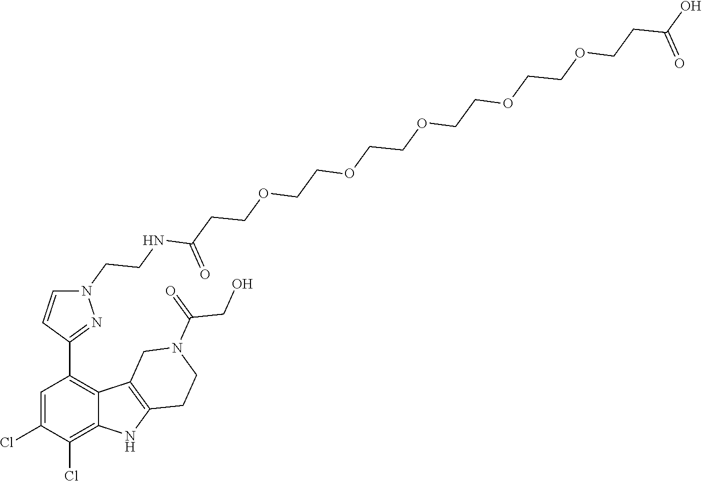 [SMILES: O=C(O)CCOCCOCCOCCOCCOCCC(=O)NCCn1ccc(-c2cc(Cl)c(Cl)c3[nH]c4c(c23)CN(C(=O)CO)CC4)n1]

Linker 55: 1-(3-(6,7-dichloro-2-(2-hydroxyacetyl)-2,3,4,5-tetrahydro-1H-pyrido[4,3-b]indol-9-yl)-1H-pyrazol-1-yl)-4-oxo-7,10,13,16,19-pentaoxa-3-azadocosan-22-oic acid. Brown solid, yield 47%. ¹H NMR (400 MHz, CD₃OD) δ 7.79-7.49 (m, 1H), 7.11 (d, J=17.3 Hz, 1H), 6.40 (dd, J=24.3, 2.3 Hz, 1H), 4.54-4.10 (m, 6H), 3.73-3.38 (m, 24H), 2.92-2.76 (m, 2H), 2.51-2.14 (m, 4H).

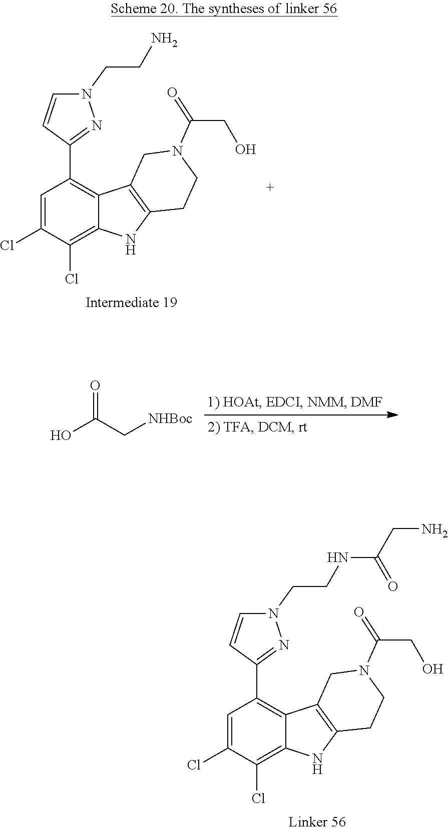 [SMILES: NCCn1ccc(-c2cc(Cl)c(Cl)c3[nH]c4c(c23)CN(C(=O)CO)CC4)n1 ; CC(C)(C)OC(=O)NCC(=O)O ; NCC(=O)NCCn1ccc(-c2cc(Cl)c(Cl)c3[nH]c4c(c23)CN(C(=O)CO)CC4)n1]

Linker 56: 2-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)acetamide To a solution of intermediate 19 (65 mg, 0.12 mmol) in DMF (0.5 mL) were added N-Boc glycine (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred overnight at rt, the resulting mixture was purified by prep-HPLC to afford crude product. The crude product was dissolved in DCM/TFA (1:1, 2 mL) followed by stirred at rt for 30 min. The resulting mixture was purified by prep-HPLC to afford title compound as yellow solid (44 mg, 63% yield). ¹H NMR (400 MHz, CD₃OD) δ 7.78-7.65 (m, 1H), 7.21 (d, J=17.2 Hz, 1H), 6.51 (dd, J=26.0, 2.0 Hz, 1H), 4.60-4.50 (m, 2H), 4.44-4.30 (m, 4H), 3.00-2.87 (m, 6H), 7.78-7.65 (m, 2H).

Linker 57-70 were synthesized following the same procedure for preparing linker 56.

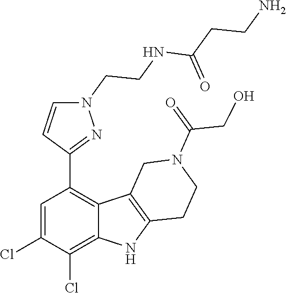 [SMILES: NCCC(=O)NCCn1ccc(-c2cc(Cl)c(Cl)c3[nH]c4c(c23)CN(C(=O)CO)CC4)n1]

Linker 57: 3-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide. Brown solid, yield 42%. ¹H NMR (400 MHz, CD₃OD) δ 7.67 (d, J=10.4 Hz, 1H), 7.17 (d, J=21.1 Hz, 1H), 6.48 (d, J=29.4 Hz, 1H), 4.53-4.31 (m, 6H), 3.73-3.70 (m, 4H), 3.21-3.07 (m, 2H), 2.94-2.86 (m, 2H), 2.62-2.56 (m, 2H).

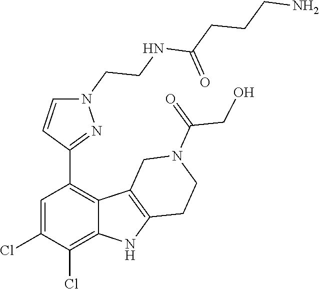 [SMILES: NCCCC(=O)NCCn1ccc(-c2cc(Cl)c(Cl)c3[nH]c4c(c23)CN(C(=O)CO)CC4)n1]

Linker 58: 4-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)butanamide. Brown solid, yield 41%. ¹H NMR (400 MHz, CD₃OD) δ 7.67 (dd, J=18.2, 2.3 Hz, 1H), 7.20 (d, J=17.4 Hz, 1H), 6.49 (dd, J=28.0, 2.3 Hz, 1H), 4.61-4.30 (m, 6H), 3.81-3.58 (m, 4H), 3.03-2.77 (m, 4H), 2.35 (t, J=7.1 Hz, 2H), 2.01-1.80 (m, 2H).

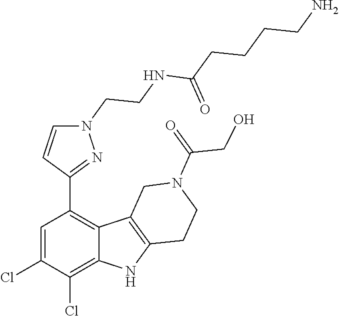 [SMILES: NCCCCC(=O)NCCn1ccc(-c2cc(Cl)c(Cl)c3[nH]c4c(c23)CN(C(=O)CO)CC4)n1]

Linker 59: 5-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)pentanamide. Brown solid, yield 44%. ¹H NMR (400 MHz, CD₃OD) δ 7.78-7.58 (m, 1H), 7.21 (d, J=14.0 Hz, 1H), 6.67-6.29 (m, 1H), 4.65-4.18 (m, 6H), 4.02-3.61 (m, 4H), 4.02-3.62 (m, 4H), 2.29-2.21 (m, 2H), 1.72-1.59 (m, 4H).

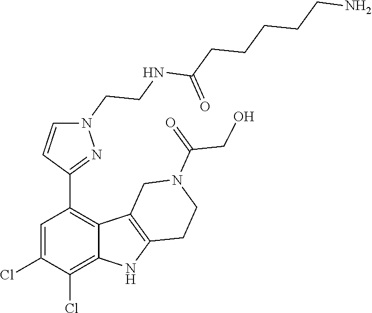 [SMILES: NCCCCCC(=O)NCCn1ccc(-c2cc(Cl)c(Cl)c3[nH]c4c(c23)CN(C(=O)CO)CC4)n1]

Linker 60: 6-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)hexanamide. Brown solid, yield 47%. ¹H NMR (400 MHz, CD₃OD) δ 7.68 (dd, J=20.8, 2.3 Hz, 1H), 7.20 (d, J=14.7 Hz, 1H), 6.50 (dd, J=24.4, 2.3 Hz, 1H), 4.65-4.30 (m, 6H), 3.75-3.66 (m, 4H), 2.98-2.84 (m, 4H), 2.30-2.02 (m, 2H), 1.65-1.53 (m, 4H), 1.44-1.24 (m, 2H).

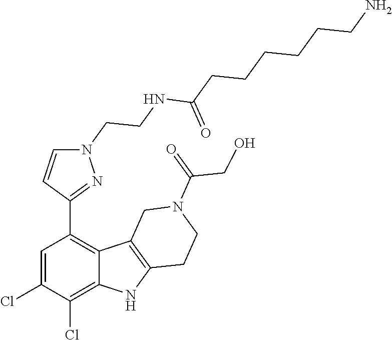 [SMILES: NCCCCCCC(=O)NCCn1ccc(-c2cc(Cl)c(Cl)c3[nH]c4c(c23)CN(C(=O)CO)CC4)n1]

Linker 61: 7-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)heptanamide. Brown solid, yield 43%. ¹H NMR (400 MHz, CD₃OD) δ 7.70-7.60 (m, 1H), 7.20 (d, J=14.3 Hz, 1H), 6.50 (dd, J=23.5, 2.3 Hz, 1H), 4.70-4.29 (m, 6H), 3.82-3.60 (m, 4H), 3.04-2.73 (m, 4H), 2.29-2.06 (m, 2H), 1.78-1.16 (m, 8H).

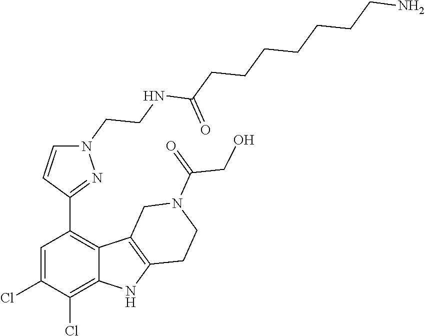 [SMILES: NCCCCCCCC(=O)NCCn1ccc(-c2cc(Cl)c(Cl)c3[nH]c4c(c23)CN(C(=O)CO)CC4)n1]

Linker 62: 8-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)octanamide. Brown solid, yield 45%. ¹H NMR (400 MHz, CD₃OD) δ 7.70-7.58 (m, 1H), 7.20 (d, J=14.1 Hz, 111), 6.49 (dd, J=24.0, 2.3 Hz, 1H), 4.70-4.29 (m, 6H), 3.78-3.62 (m, 4H), 3.05-2.76 (m, 4H), 2.32-2.05 (m, 2H), 1.69-1.12 (m, 10H).

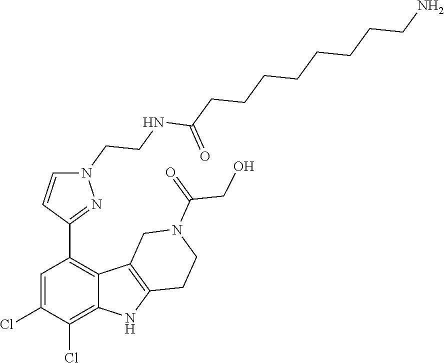 [SMILES: NCCCCCCCCC(=O)NCCn1ccc(-c2cc(Cl)c(Cl)c3[nH]c4c(c23)CN(C(=O)CO)CC4)n1]

Linker 63: 9-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)nonanamide. Brown solid, yield 46%. ¹H NMR (400 MHz, CD₃OD) δ 7.66 (dd, J=20.3, 2.3 Hz, 1H), 7.20 (d, J=13.2 Hz, 1H), 6.49 (dd, J=24.0, 2.3 Hz, 1H), 4.69-4.16 (m, 6H), 3.98-3.61 (m, 4H), 3.01-2.74 (m, 4H), 2.26-2.08 (m, 2H), 1.77-1.48 (m, 4H), 1.44-1.15 (m, 8H).

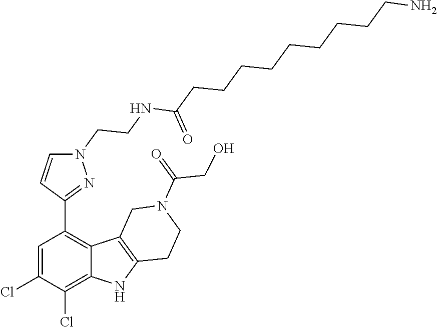 [SMILES: NCCCCCCCCCC(=O)NCCn1ccc(-c2cc(Cl)c(Cl)c3[nH]c4c(c23)CN(C(=O)CO)CC4)n1]

Linker 64: 10-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)decanamide. Brown solid, yield 49%. ¹H NMR (400 MHz, CD₃OD) δ 7.66 (dd, J=21.3, 2.3 Hz, 1H), 7.21 (d, J=12.8 Hz, 1H), 6.49 (dd, J=24.3, 2.3 Hz, 1H), 4.56-4.16 (m, 6H), 3.88-3.49 (m, 4H), 2.81 (dq, J=28.2, 7.7, 6.7 Hz, 4H), 2.05 (dt, J=16.3, 7.5 Hz, OH), 1.60-1.03 (m, 14H).

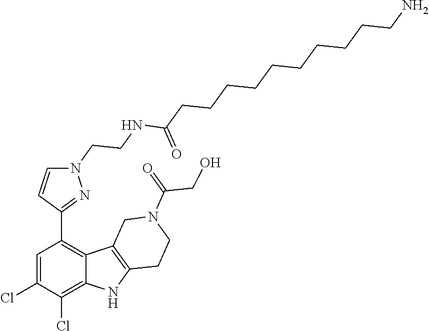 [SMILES: NCCCCCCCCCCC(=O)NCCn1ccc(-c2cc(Cl)c(Cl)c3[nH]c4c(c23)CN(C(=O)CO)CC4)n1]

Linker 65: 11-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)undecanamide. Brown solid, yield 44%. 1H NMR (400 MHz, CD₃OD) δ 7.83-7.54 (m, 1H), 7.34-7.07 (m, 1H), 6.49 (dd, J=24.6, 2.3 Hz, 1H), 4.56-4.16 (m, 6H), 3.88-3.49 (m, 4H), 2.94-2.67 (m, 4H), 2.15-1.93 (m, 2H), 1.60-1.03 (m, 16H).

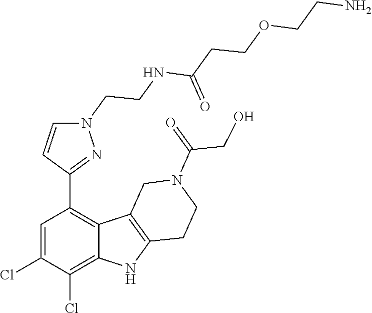 [SMILES: NCCOCCC(=O)NCCn1ccc(-c2cc(Cl)c(Cl)c3[nH]c4c(c23)CN(C(=O)CO)CC4)n1]

Linker 66: 3-(2-aminoethoxy)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide. Brown solid, yield 42%. 1H NMR (400 MHz, CD₃OD) δ 7.69 (dd, J=17.1, 2.3 Hz, 1H), 7.21 (d, J=16.7 Hz, 1H), 6.51 (dd, J=26.1, 2.3 Hz, 1H), 4.62-4.29 (m, 6H), 3.83-3.55 (m, 8H), 3.18-2.85 (m, 4H), 2.61-2.23 (m, 2H).

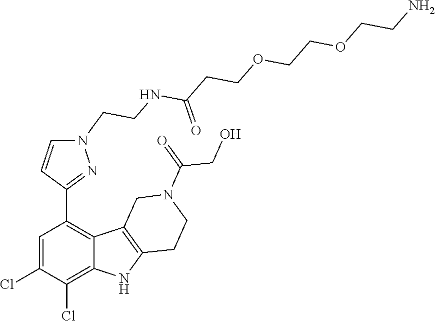 [SMILES: NCCOCCOCCC(=O)NCCn1ccc(-c2cc(Cl)c(Cl)c3[nH]c4c(c23)CN(C(=O)CO)CC4)n1]

Linker 67: 3-(2-(2-aminoethoxy)ethoxy)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide. Brown solid, yield 45%. ¹H NMR (400 MHz, CD₃OD) δ 7.73-7.67 (m, 1H), 7.21 (d, J=16.0 Hz, 1H), 6.51 (dd, J=24.7, 2.3 Hz, 1H), 4.65-4.30 (m, 6H), 3.80-3.44 (m, 12H), 3.13-2.85 (m, 4H), 2.54-2.34 (m, 2H).

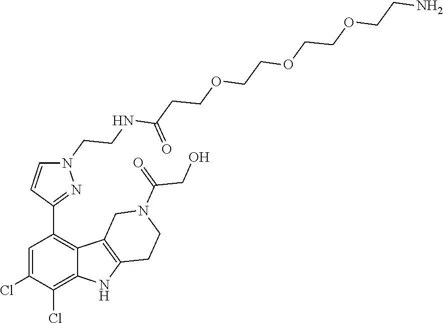 [SMILES: NCCOCCOCCOCCC(=O)NCCn1ccc(-c2cc(Cl)c(Cl)c3[nH]c4c(c23)CN(C(=O)CO)CC4)n1]

Linker 68: 3-(2-(2-(2-aminoethoxy)ethoxy)ethoxy)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide. Brown solid, yield 46%. ¹H NMR (400 MHz, CD₃OD) δ 7.73-7.68 (m, 1H), 7.22 (d, J=15.6 Hz, 1H), 6.51 (dd, J=25.8, 2.3 Hz, 1H), 4.65-4.31 (m, 6H), 3.78-3.48 (m, 16H), 3.18-2.87 (m, 4H), 2.52-2.28 (m, 2H).

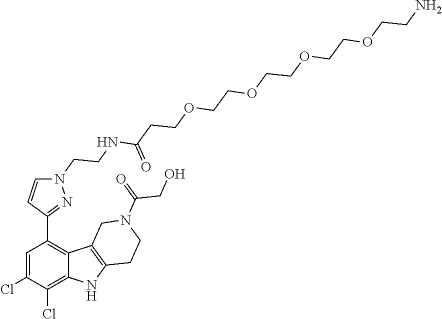 [SMILES: NCCOCCOCCOCCOCCC(=O)NCCn1ccc(-c2cc(Cl)c(Cl)c3[nH]c4c(c23)CN(C(=O)CO)CC4)n1]

Linker 69: 1-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3,6,9,12-tetraoxapentadecan-15-amide. Brown solid, yield 42%. ¹H NMR (400 MHz, CD₃OD) δ 7.68 (dd, J=12.8, 2.3 Hz, 1H), 7.22 (d, J=16.5 Hz, 1H), 6.51 (dd, J=25.3, 2.3 Hz, 1H), 4.65-4.31 (m, 6H), 3.78-3.51 (m, 20H), 3.17-2.82 (m, 4H), 2.57-2.31 (m, 2H).

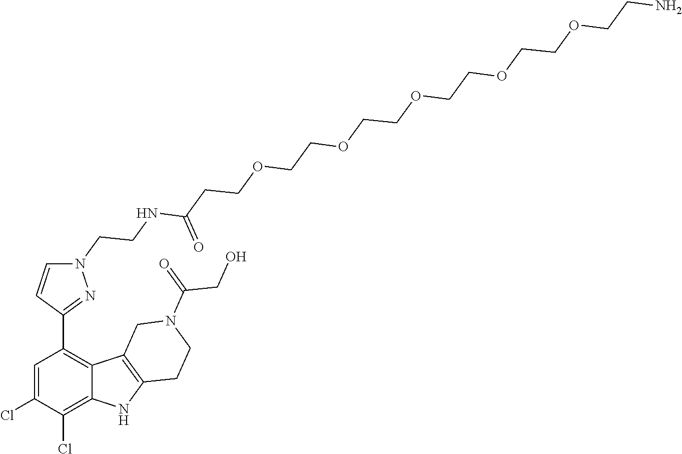 [SMILES: NCCOCCOCCOCCOCCOCCC(=O)NCCn1ccc(-c2cc(Cl)c(Cl)c3[nH]c4c(c23)CN(C(=O)CO)CC4)n1]

Linker 70: 1-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3,6,9,12,15-pentaoxaoctadecan-18-amide. Brown solid, yield 44%. ¹H NMR (400 MHz, CD₃OD) δ 7.74-7.66 (m, 1H), 7.22 (d, J=17.0 Hz, 1H), 6.51 (dd, J=25.1, 2.3 Hz, 1H), 4.65-4.30 (m, 6H), 3.79-3.48 (m, 24H), 3.18-3.04 (m, 4H), 2.55-2.34 (m, 2H).

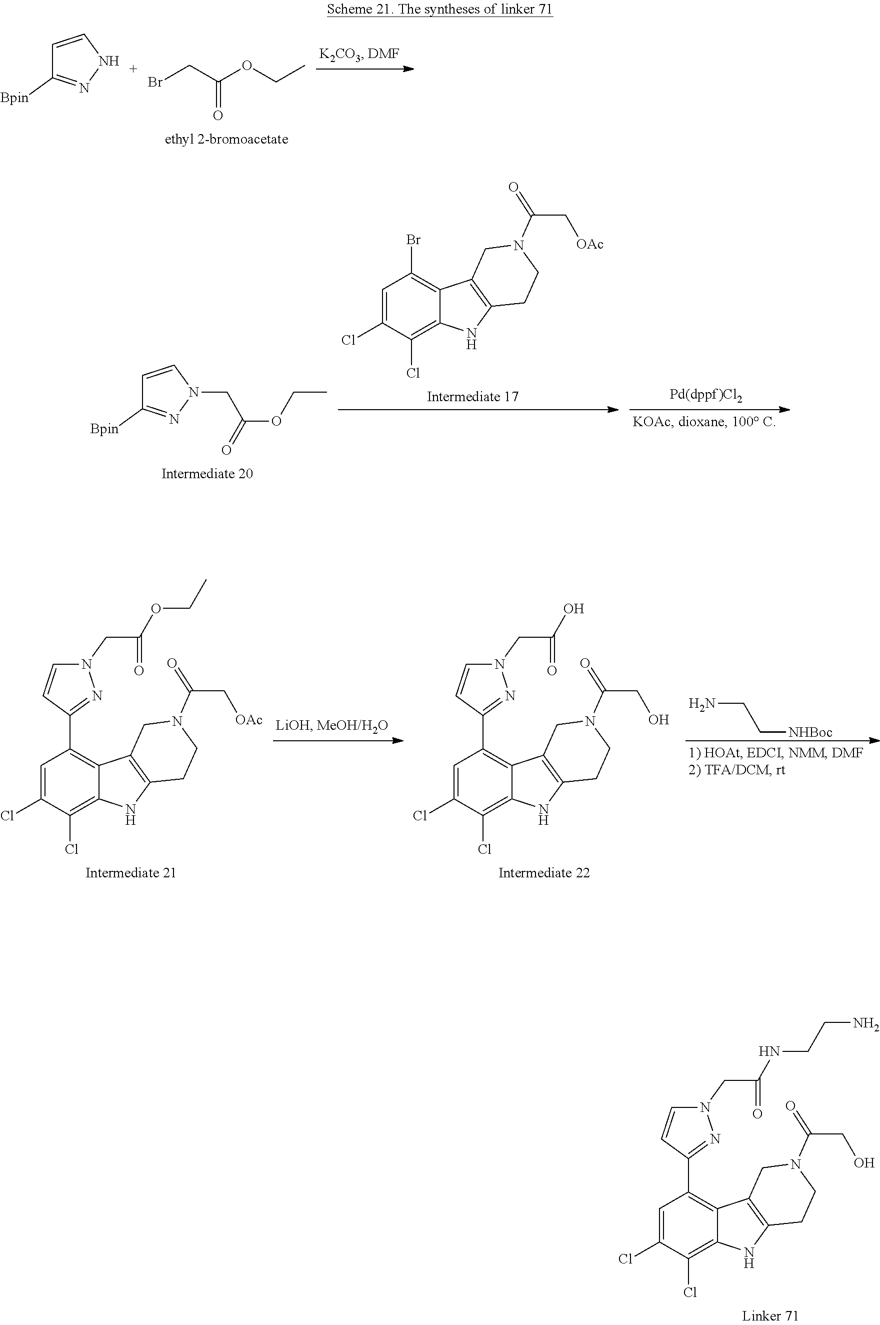

Linker 71: N-(2-aminoethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (XH168-108) To a solution of 3-(4,4,5,5-tetramethyl-1,3,2-dioxaborolan-2-yl)-1H-pyrazole (7.3 g, 37.6 mmol, 1.0 eq) in MeCN (30 ml) was added K₂CO₃ (15.6 g, 112.9 mmol, 3.0 eq) and tert-butyl 2-bromoacetate (7.5 g, 45.1 mmol, 1.2 eq). The mixture was stirred at 80° C. for 6 h. After that, water was added and the mixture was extracted with ethyl acetate. The organic layer was collected, and excess solvent was removed to get intermediate 20 and used for the next step directly without further purification.

To a solution of intermediate 17 (1.05 g, 2.5 mmol, 1 eq) and intermediate 20 (2.7 g, 9.5 mmol, 3.8 eq) in dioxane (40 ml) was added KOAc (981.4 mg, 10.0 mmol, 4.0 eq) and Pd(dppf)Cl₂—CH₂Cl₂ (408 mg, 0.5 mmol, 0.2 eq). The mixture was stirred at 100° C. for 6 h under N₂. Ater cooling down to room temperature, the reaction mixture was concentrated under reduced pressure and purified by column chromatography (SiO₂, DCM/MeOH=10:1) to give intermediate 21 as a brown oil, 68% yield.

To a solution of intermediate 21 (840 mg, 1.7 mmol, 1 eq) in MeOH (20 ml) and H₂O (5 ml) was added LiOH (214 mg, 5.1 mmol, 3 eq). After stirred at rt for 10 min, excess MeOH was removed, resulted residue was diluted with H₂O (2 mL) and the pH value was adjusted to 6 with HCl aq. (1M) followed by purified by prep-HPLC to yield intermediate 22 as a yellow solid (456 mg, 33% in 2 steps). ¹H NMR (400 MHz, CD₃OD) δ 7.72 (d, J=2.3 Hz, 1H), 7.24 (s, 1H), 6.53 (d, J=2.3 Hz, 1H), 4.89-4.84 (m, 2H), 4.63-4.49 (m, 2H), 4.37-4.26 (m, 2H), 4.01-3.70 (m, 2H), 3.01-2.85 (m, 2H).

To a solution of intermediate 22 (65 mg, 0.12 mmol) in DMF (0.5 mL) were added tert-butyl (2-aminoethyl)carbamate (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred for overnight at rt, the resulting mixture was purified by prep-HPLC to afford crude product. The crude product was dissolved in DCM/TFA (1:1, 2 mL) followed by stirred at rt for 30 min. The resulting mixture was purified by prep-HPLC to afford titled compound as yellow solid (44 mg, 63% yield). ¹H NMR (400 MHz, CD₃OD) δ 7.77 (s, 1H), 7.14 (s, 1H), 6.51 (s, 1H), 5.02-4.97 (m, 2H), 4.58 (s, 1H), 4.32 (s, 1H), 4.17 (s, 1H), 3.92 (s, 1H), 3.64-3.53 (m, 1H), 3.52-3.49 (m, 2H),), 3.29-3.23 (m, 1H), 3.06 (s, 2H), 2.89-2.81 (m, 2H).

Linker 72-84 were synthesized following the same procedure for preparing linker 71.

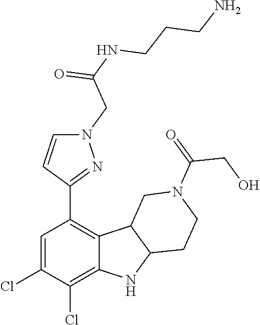 [SMILES: NCCCNC(=O)Cn1ccc(-c2cc(Cl)c(Cl)c3c2C2CN(C(=O)CO)CCC2N3)n1]

Linker 72: N-(3-aminopropyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-109). Yellow solid, 53% yield. ¹H NMR (400 MHz, CD₃OD) δ 7.74 (s, 1H), 7.19 (s, 1H), 6.54 (s, 1H), 4.96 (s, 2H), 4.64 (s, 1H), 4.41 (s, 1H), 4.35 (s, 1H), 4.21 (s, 1H), 3.91 (s, 1H), 3.67 (s, 1H), 3.34 (s, 3H), 2.97-2.87 (m, 3H), 1.92-1.75 (m, 2H).

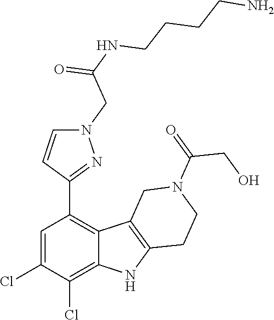 [SMILES: NCCCCNC(=O)Cn1ccc(-c2cc(Cl)c(Cl)c3[nH]c4c(c23)CN(C(=O)CO)CC4)n1]

Linker 73: N-(4-aminobutyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-112). Yellow solid, 37% yield. 1H NMR (400 MHz, CD₃OD) δ 7.77 (s, 1H), 7.22 (s, 1H), 6.58 (s, 1H), 4.73 (s, 2H), 4.58-4.44 (m, 1H), 4.10-3.96 (m, 1H), 3.93-3.80 (m, 1H), 3.68-3.56 (m, 1H), 3.33-3.18 (m, 3H), 2.86 (s, 3H), 1.60-1.52 (m, 4H), 1.26-1.22 (m, 2H).

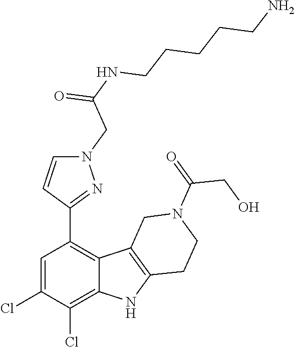 [SMILES: NCCCCCNC(=O)Cn1ccc(-c2cc(Cl)c(Cl)c3[nH]c4c(c23)CN(C(=O)CO)CC4)n1]

Linker 74: N-(5-aminopentyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-113). Yellow solid, yield 32% ¹H NMR (400 MHz, CD₃OD) δ 7.73 (s, 1H), 7.20 (s, 1H), 6.54 (s, 1H), 4.93 (s, 2H), 4.69-4.59 (m, 1H), 4.45-4.37 (m, 1H), 4.35-4.30 (m, 1H), 4.26-4.20 (m, 1H), 3.94-3.90 (m, 1H), 3.79-3.64 (m, 1H), 3.25-3.18 (m, 2H), 2.93-2.86 (m, 4H), 1.64-1.54 (m, 4H), 1.41-1.37 (m, 2H).

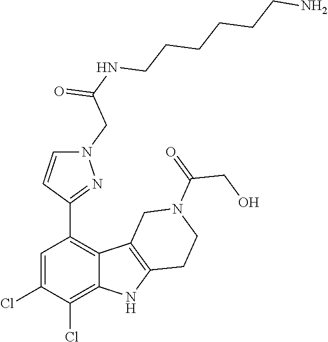 [SMILES: NCCCCCCNC(=O)Cn1ccc(-c2cc(Cl)c(Cl)c3[nH]c4c(c23)CN(C(=O)CO)CC4)n1]

Linker 75:N-(6-aminohexyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-114). Yellow solid, 38% yield. 1H NMR (400 MHz, CD₃OD) δ 7.73 (s, 1H), 7.20 (s, 1H), 6.54 (s, 1H), 4.93 (s, 2H), 4.71-4.57 (m, 1H), 4.47-4.37 (m, 1H), 4.35-4.28 (m, 1H), 4.28-4.16 (m, 1H), 4.00-3.79 (m, 1H), 3.70-3.67 (m, 1H), 3.30-3.21 (m, 2H), 2.92-2.85 (m, 4H), 1.62-1.51 (m, 4H), 1.38-1.33 (m, 4H).

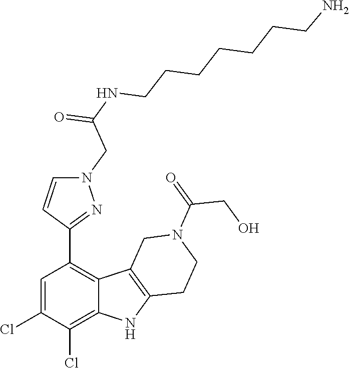 [SMILES: NCCCCCCCNC(=O)Cn1ccc(-c2cc(Cl)c(Cl)c3[nH]c4c(c23)CN(C(=O)CO)CC4)n1]

Linker 76: N-(7-aminoheptyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-115). Yellow solid, 35% yield. ¹H NMR (400 MHz, CD₃OD) δ 7.74 (s, 1H), 7.20 (s, 1H), 6.54 (s, 1H), 4.92 (s, 2H), 4.68-4.56 (m, 1H), 4.45-4.34 (m, 1H), 4.35-4.28 (m, 1H), 4.26-4.18 (m, 1H), 3.92-3.89 (m, 1H), 3.69-3.66 (m, 1H), 3.23-3.18 (m, 2H), 2.93-2.82 (m, 4H), 1.62-1.47 (m, 4H), 1.32-1.29 (m, 6H).

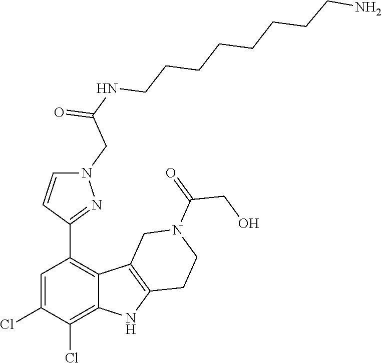 [SMILES: NCCCCCCCCNC(=O)Cn1ccc(-c2cc(Cl)c(Cl)c3[nH]c4c(c23)CN(C(=O)CO)CC4)n1]

Linker 77: N-(8-aminooctyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-116). Yellow solid, 33% yield. 1H NMR (400 MHz, CD₃OD) δ 7.73 (s, 1H), 7.19 (s, 1H), 6.53 (s, 1H), 4.92 (s, 2H), 4.67-4.60 (m, 1H), 4.43-4.37 (m, 1H), 4.36-4.28 (m, 1H), 4.26-4.18 (m, 1H), 3.99-3.83 (m, 1H), 3.75-3.62 (m, 1H), 3.27-3.16 (m, 2H), 2.96-2.74 (m, 4H), 1.71-1.42 (m, 4H), 1.41-1.16 (m, 8H).

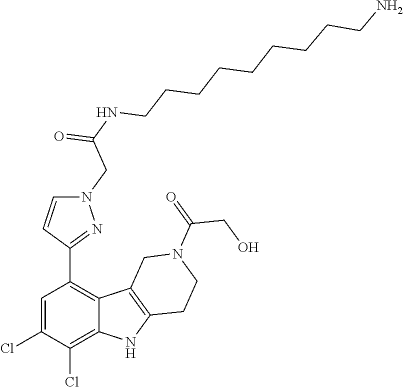 [SMILES: NCCCCCCCCCNC(=O)Cn1ccc(-c2cc(Cl)c(Cl)c3[nH]c4c(c23)CN(C(=O)CO)CC4)n1]

Linker 78: N-(9-aminononyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-117). Yellow solid, 79% yield. 1H NMR (400 MHz, CD₃OD) δ 7.72 (s, 1H), 7.17 (s, 1H), 6.52 (s, 1H), 4.92 (s, 2H), 4.74-4.56 (m, 1H), 4.50-4.38 (m, 1H), 4.31-4.21 (m, 2H), 4.00-3.84 (m, 1H), 3.75-3.59 (m, 1H), 3.27-3.09 (m, 2H), 3.03-2.68 (m, 4H), 1.61-1.46 (m, 4H), 1.33-1.20 (m, 10H).

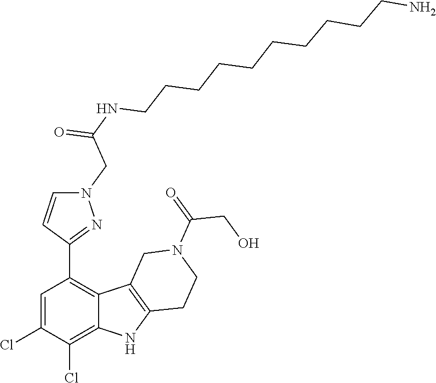 [SMILES: NCCCCCCCCCCNC(=O)Cn1ccc(-c2cc(Cl)c(Cl)c3[nH]c4c(c23)CN(C(=O)CO)CC4)n1]

Linker 79: N-(10-aminodecyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-118). Yellow solid, 39 mg, 47% yield. ¹H NMR (400 MHz, CD₃OD) δ 7.73 (s, 1H), 7.20 (s, 1H), 6.54 (s, 1H), 4.92 (s, 2H), 4.71-4.60 (m, 1H), 4.49-4.40 (m, 1H), 4.38-4.31 (m, 1H), 4.31-4.18 (m, 1H), 3.97-3.91 (m, 1H), 3.76-3.62 (m, 1H), 3.28-3.11 (m, 2H), 3.03-2.79 (m, 4H), 1.63-1.45 (m, 4H), 1.35-1.21 (m, 12H).

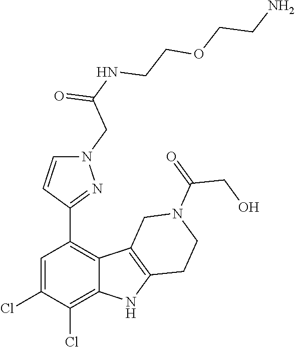 [SMILES: NCCOCCNC(=O)Cn1ccc(-c2cc(Cl)c(Cl)c3[nH]c4c(c23)CN(C(=O)CO)CC4)n1]

Linker 80: N-(2-(2-aminoethoxy)ethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-119). Yellow solid, 53% yield. ¹H NMR (400 MHz, CD₃OD) δ 7.72 (s, 1H), 7.18 (s, 1H), 6.52 (s, 1H), 4.97-4.96 (m, 2H), 4.72-4.60 (m, 1H), 4.41-4.35 (m, 2H), 4.29-4.14 (m, 1H), 3.91-3.88 (m, 1H), 3.66-3.63 (m, 3H), 3.60-3.51 (m, 2H), 3.51-3.39 (m, 2H), 3.16-3.00 (m, 2H), 2.99-2.78 (m, 2H).

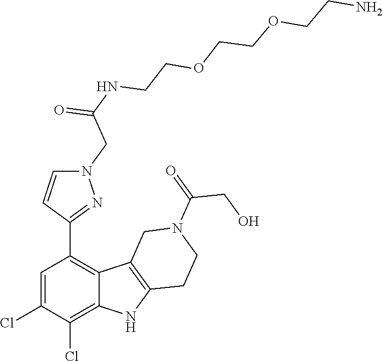 [SMILES: NCCOCCOCCNC(=O)Cn1ccc(-c2cc(Cl)c(Cl)c3[nH]c4c(c23)CN(C(=O)CO)CC4)n1]

Linker 81: N-(2-(2-(2-aminoethoxy)ethoxy)ethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-120). Yellow solid, 46% yield. ¹H NMR (400 MHz, CD₃OD) δ 7.72 (s, 1H), 7.17 (s, 1H), 6.51 (s, 1H), 4.96-4.94 (m, 2H), 4.68-4.58 (m, 1H), 4.50-4.39 (m, 1H), 4.39-4.31 (m, 1H), 4.30-4.22 (m, 1H), 3.91-3.88 (m, 1H), 3.69-3.48 (m, 9H), 3.47-3.36 (m, 2H), 3.12-2.97 (m, 2H), 2.95-2.79 (m, 2H).

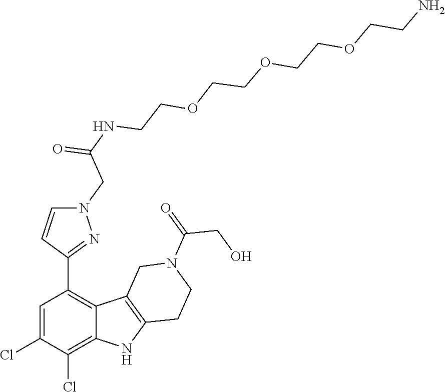 [SMILES: NCCOCCOCCOCCNC(=O)Cn1ccc(-c2cc(Cl)c(Cl)c3[nH]c4c(c23)CN(C(=O)CO)CC4)n1]

Linker 82: N-(2-(2-(2-(2-aminoethoxy)ethoxy)ethoxy)ethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-121). Yellow solid, 30% yield. ¹H NMR (400 MHz, CD₃OD) δ 7.73 (s, 1H), 7.18 (s, 1H), 6.53 (s, 1H), 4.96-4.90 (m, 2H), 4.68-4.60 (m, 1H), 4.54-4.43 (m, 1H), 4.38-4.30 (m, 1H), 4.30-4.19 (m, 1H), 4.00-3.84 (m, 1H), 3.73-3.45 (m, 13H), 3.43-3.35 (m, 2H), 3.07-3.03 (m, 2H), 2.92-2.85 (m, 2H).

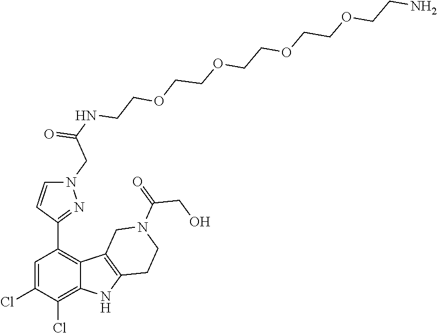 [SMILES: NCCOCCOCCOCCOCCNC(=O)Cn1ccc(-c2cc(Cl)c(Cl)c3[nH]c4c(c23)CN(C(=O)CO)CC4)n1]

Linker 83: N-(14-amino-3,6,9,12-tetraoxatetradecyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-122). Yellow solid, 29% yield. ¹H NMR (400 MHz, CD₃OD) δ 7.75 (s, 1H), 7.22 (s, 1H), 6.56 (s, 1H), 4.92 (s, 2H), 4.70-4.62 (m, 1H), 4.48-4.46 (m, 1H), 4.39-4.32 (m, 1H), 4.29-4.16 (m, 1H), 4.00-3.89 (m, 1H), 3.70-3.40 (m, 17H), 3.44-3.35 (m, 2H), 3.13-3.02 (m, 2H), 2.94-2.88 (m, 2H).

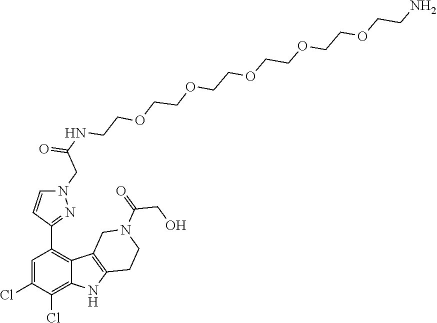 [SMILES: NCCOCCOCCOCCOCCOCCNC(=O)Cn1ccc(-c2cc(Cl)c(Cl)c3[nH]c4c(c23)CN(C(=O)CO)CC4)n1]

Linker 84: N-(17-amino-3,6,9,12,15-pentaoxaheptadecyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-123) Yellow solid, 32% yield. ¹H NMR (400 MHz, CD₃OD) δ 7.76 (s, 1H), 7.23 (s, 1H), 6.57 (s, 1H), 4.92 (s, 2H), 4.71-4.61 (m, 1H), 4.51-4.41 (m, 1H), 4.38-4.29 (m, 1H), 4.29-4.18 (m, 1H), 3.99-3.89 (m, 1H), 3.71-3.48 (m, 21H), 3.46-3.36 (m, 2H), 3.12-2.98 (m, 2H), 2.98-2.83 (m, 2H).

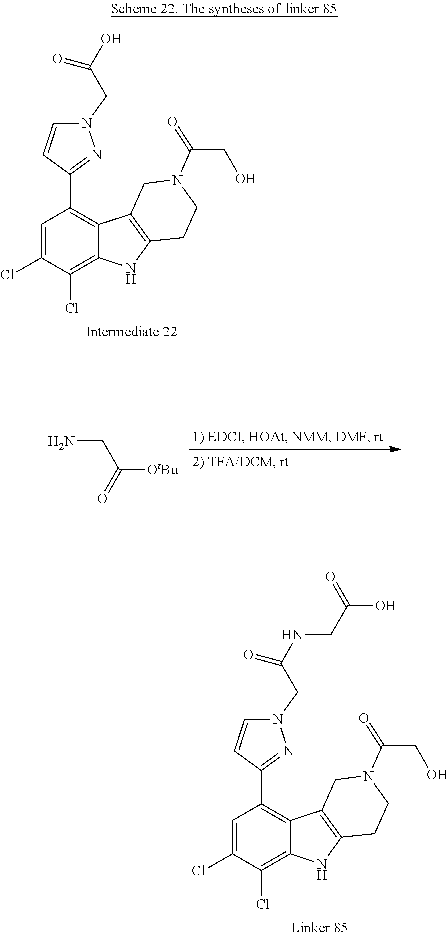 [SMILES: O=C(O)Cn1ccc(-c2cc(Cl)c(Cl)c3[nH]c4c(c23)CN(C(=O)CO)CC4)n1 ; CC(C)(C)OC(=O)CN ; O=C(O)CNC(=O)Cn1ccc(-c2cc(Cl)c(Cl)c3[nH]c4c(c23)CN(C(=O)CO)CC4)n1]

Linker 85: N-(2-aminoethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (XH168-108). To a solution of Intermediate 22 (50 mg, 0.1 mmol) in DMF (1 mL) were added tert-butyl glycinate (13.1 mg, 0.1 mmol, 1.0 equiv), 1-ethyl-3-(3-dimethylaminopropyl)carbodiimide (EDCI, 28.7 mg, 0.15 mmol, 1.5 equiv), 1-hydroxy-7-azabenzo-triazole (HOAt, 20.4 mg, 0.15 mmol, 1.5 equiv), and N-methylmorpholine (NMM, 30.3 mg, 0.3 mmol, 3.0 equiv). After being stirred at rt overnight, the resulting mixture was purified by prep-HPLC to afford crude product. The crude product was dissolved in DCM/TFA (1:1, 2 mL) followed by stirred at rt for 30 min. The resulting mixture was purified by prep-HPLC to afford titled compound as white solid (16 mg, 32% yield). ¹H NMR (400 MHz, CD₃OD) δ 7.76 (s, 1H), 7.21 (s, 1H), 6.55 (s, 1H), 5.07-4.98 (m, 2H), 4.68-4.56 (m, 1H), 4.49-4.39 (m, 1H), 4.35-4.24 (m, 1H), 4.25-4.14 (m, 1H), 4.04-3.86 (m, 3H), 3.81-3.63 (m, 1H), 3.03-2.75 (m, 2H).

Linkers 86-99 were synthesized following the same procedure for preparing linker 85.

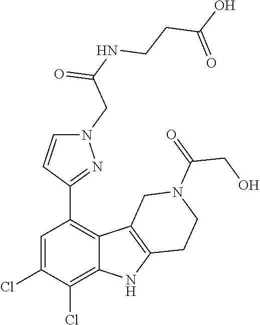 [SMILES: O=C(O)CCNC(=O)Cn1ccc(-c2cc(Cl)c(Cl)c3[nH]c4c(c23)CN(C(=O)CO)CC4)n1]

Linker 86: 3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)propanoic acid (XH181-71). White solid, 49% yield. White solid, 49%. ¹H NMR (400 MHz, CD₃OD) δ 7.75 (s, 1H), 7.23 (s, 1H), 6.55 (s, 1H), 4.94 (s, 2H), 4.69-4.59 (m, 1H), 4.49-4.39 (m, 1H), 4.36-4.26 (m, 1H), 4.26-4.17 (m, 1H), 4.03-3.87 (m, 1H), 3.83-3.64 (m, 1H), 3.58-3.40 (m, 2H), 3.02-2.76 (m, 2H), 2.60-2.45 (m, 2H).

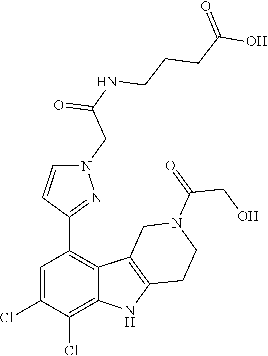 [SMILES: O=C(O)CCCNC(=O)Cn1ccc(-c2cc(Cl)c(Cl)c3[nH]c4c(c23)CN(C(=O)CO)CC4)n1]

Linker 87: 4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)butanoic acid (XH181-74). White solid, 70% yield. ¹H NMR (400 MHz, CD₃OD) δ 7.76 (s, 1H), 7.22 (s, 1H), 6.55 (s, 1H), 4.97-4.93 (m, 2H), 4.69-4.59 (m, 1H), 4.49-4.39 (m, 1H), 4.36-4.26 (m, 1H), 4.25-4.15 (m, 1H), 4.00-3.88 (m, 1H), 3.79-3.69 (m, 1H), 3.23-3.17 (m, 2H), 3.01-2.75 (m, 2H), 2.41-2.25 (m, 2H), 1.69-1.43 (m, 2H).

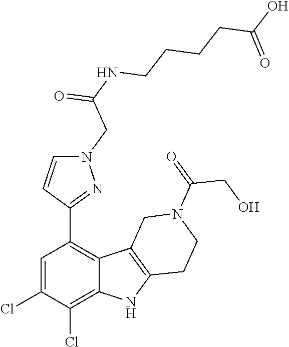 [SMILES: O=C(O)CCCCNC(=O)Cn1ccc(-c2cc(Cl)c(Cl)c3[nH]c4c(c23)CN(C(=O)CO)CC4)n1]

Linker 88: 5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)pentanoic acid (XH181-75). White solid, 47% yield. ¹H NMR (400 MHz, CD₃OD) δ 7.75 (s, 1H), 7.22 (s, 1H), 6.55 (s, 1H), 5.01-4.90 (m, 2H), 4.73-4.61 (m, 1H), 4.48-4.38 (m, 1H), 4.37-4.28 (m, 1H), 4.26-4.14 (m, 1H), 4.00-3.88 (m, 1H), 3.81-3.69 (m, 1H), 3.27-3.19 (m, 2H), 3.01-2.78 (m, 2H), 2.41-2.24 (m, 2H), 1.72-1.45 (m, 4H).

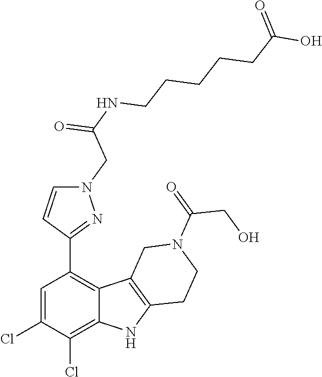 [SMILES: O=C(O)CCCCCNC(=O)Cn1ccc(-c2cc(Cl)c(Cl)c3[nH]c4c(c23)CN(C(=O)CO)CC4)n1]

Linker 89: 6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)hexanoic acid (XH181-151). Yellow solid, 25% yield. ¹H NMR (400 MHz, CD₃OD) δ 7.74 (s, 1H), 7.21 (s, 1H), 6.55 (s, 1H), 4.99-4.91 (m, 2H), 4.71-4.60 (m, 1H), 4.50-4.37 (m, 1H), 4.37-4.26 (m, 1H), 4.27-4.14 (m, 1H), 4.02-3.85 (m, 1H), 3.79-3.64 (m, 1H), 3.28-3.15 (m, 2H), 3.03-2.79 (m, 2H), 2.33-2.14 (m, 2H), 1.69-1.43 (m, 4H), 1.43-1.24 (m, 2H).

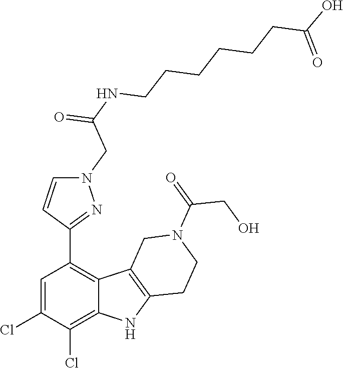 [SMILES: O=C(O)CCCCCCNC(=O)Cn1ccc(-c2cc(Cl)c(Cl)c3[nH]c4c(c23)CN(C(=O)CO)CC4)n1]

Linker 90: 7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)heptanoic acid (XH181-152). Yellow solid, 27% yield. Yellow solid, 27%. ¹H NMR (400 MHz, CD₃OD) δ 7.74 (s, 1H), 7.21 (s, 1H), 6.54 (s, 1H), 4.99-4.90 (m, 2H), 4.72-4.62 (m, 1H), 4.49-4.39 (m, 1H), 4.37-4.27 (m, 1H), 4.26-4.12 (m, 1H), 4.03-3.89 (m, 1H), 3.82-3.65 (m, 1H), 3.27-3.14 (m, 2H), 3.01-2.81 (m, 2H), 2.33-2.15 (m, 2H), 1.66-1.42 (m, 4H), 1.41-1.22 (m, 4H).

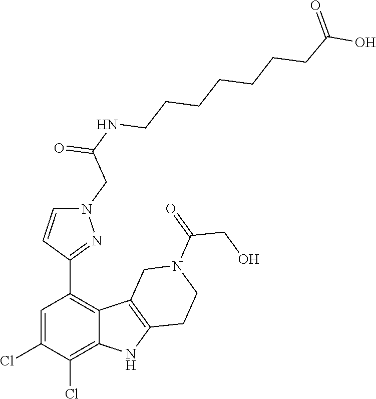 [SMILES: O=C(O)CCCCCCCNC(=O)Cn1ccc(-c2cc(Cl)c(Cl)c3[nH]c4c(c23)CN(C(=O)CO)CC4)n1]

Linker 91: 8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)octanoic acid (XH181-81). White solid, 67% yield. ¹H NMR (400 MHz, CD₃OD) δ 7.75 (s, 1H), 7.23 (s, 1H), 6.55 (s, 1H), 4.99-4.89 (m, 2H), 4.71-4.59 (m, 1H), 4.48-4.36 (m, 1H), 4.35-4.26 (m, 1H), 4.24-4.15 (m, 1H), 4.00-3.87 (m, 1H), 3.82-3.65 (m, 1H), 3.26-3.16 (m, 2H), 3.01-2.77 (m, 2H), 2.36-2.18 (m, 2H), 1.67-1.42 (m, 4H), 1.39-1.26 (m, 6H).

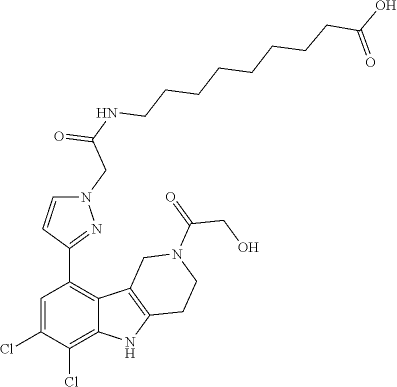 [SMILES: O=C(O)CCCCCCCCNC(=O)Cn1ccc(-c2cc(Cl)c(Cl)c3[nH]c4c(c23)CN(C(=O)CO)CC4)n1]

Linker 92: 9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)nonanoic acid (XH181-82). White solid, 54% yield. ¹H NMR (400 MHz, CD₃OD) δ 7.74 (s, 1H), 7.21 (s, 1H), 6.54 (s, 1H), 5.03-4.90 (m, 2H), 4.71-4.53 (m, 1H), 4.49-4.38 (m, 1H), 4.37-4.26 (m, 1H), 4.26-4.12 (m, 1H), 4.02-3.87 (m, 1H), 3.78-3.63 (m, 1H), 3.27-3.15 (m, 2H), 2.99-2.81 (m, 2H), 2.26-2.16 (m, 2H), 1.62-1.43 (m 4H), 1.42-1.19 (m, 8H).

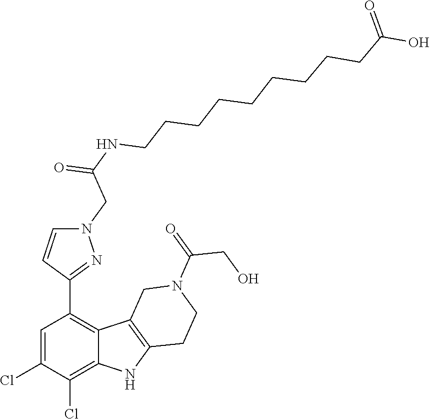 [SMILES: O=C(O)CCCCCCCCCNC(=O)Cn1ccc(-c2cc(Cl)c(Cl)c3[nH]c4c(c23)CN(C(=O)CO)CC4)n1]

Linker 93: 10-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)decanoic acid Yellow solid, 50%. ¹H NMR (400 MHz, CD₃OD) δ 7.77 (s, 1H), 7.24 (s, 1H), 6.57 (s, 1H), 5.06-4.93 (m, 2H), 4.74-4.60 (m, 1H), 4.48-4.38 (m, 1H), 4.39-4.30 (m, 1H), 4.28-4.14 (m, 1H), 3.99-3.87 (m, 1H), 3.81-3.69 (m, 1H), 3.28-3.14 (m, 2H), 3.08-2.78 (m, 2H), 2.38-2.09 (m, 2H), 1.72-1.40 (m, 4H), 1.39-1.07 (m, 10H).

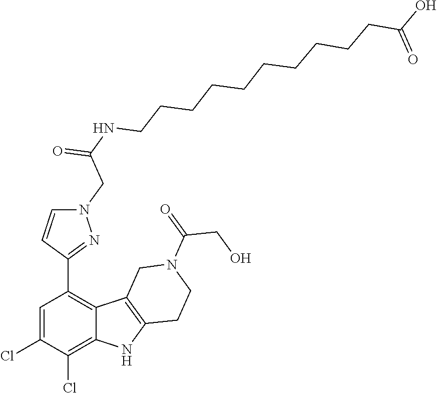 [SMILES: O=C(O)CCCCCCCCCCNC(=O)Cn1ccc(-c2cc(Cl)c(Cl)c3[nH]c4c(c23)CN(C(=O)CO)CC4)n1]

Linker 94: 11-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)undecanoic acid Yellow solid, 52%. ¹H NMR (400 MHz, CD₃OD) δ 7.76 (s, 1H), 7.23 (s, 1H), 6.56 (s, 1H), 5.05-4.92 (m, 2H), 4.75-4.60 (m, 1H), 4.47-4.37 (m, 1H), 4.35-4.25 (m, 1H), 4.24-4.10 (m, 1H), 4.00-4.87 (m, 1H), 3.79-3.70 (m, 1H), 3.27-3.15 (m, 2H), 2.92 (m, 2H), 2.26 (m, 2H), 1.53 (m, 4H), 1.35-1.14 (m, 12H).

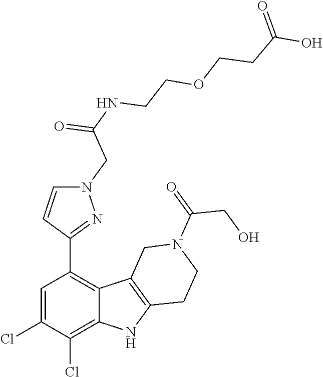 [SMILES: O=C(O)CCOCCNC(=O)Cn1ccc(-c2cc(Cl)c(Cl)c3[nH]c4c(c23)CN(C(=O)CO)CC4)n1]

Linker 95: 3-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)propanoic acid (XH181-153). White solid, 35% yield. ¹H NMR (400 MHz, CD₃OD) δ 7.75 (s, 1H), 7.21 (s, 1H), 6.55 (s, 1H), 5.01-4.92 (m, 2H), 4.68-4.58 (m, 1H), 4.46-4.39 (m, 1H), 4.34-4.27 (m, 1H), 4.26-4.11 (m, 1H), 3.96-3.87 (m, 1H), 3.77-3.56 (m, 3H), 3.59-3.47 (m, 2H), 3.46-3.36 (m, 2H), 3.01-2.79 (m, 2H), 2.63-2.38 (m, 2H).

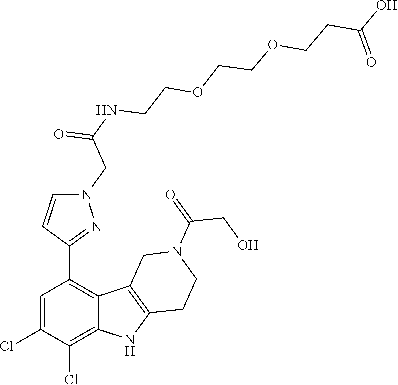 [SMILES: O=C(O)CCOCCOCCNC(=O)Cn1ccc(-c2cc(Cl)c(Cl)c3[nH]c4c(c23)CN(C(=O)CO)CC4)n1]

Linker 96: 3-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethoxy)propanoic acid (XH181-154). Yellow solid, 47% yield. ¹H NMR (400 MHz, CD₃OD) δ 7.75 (s, 1H), 7.22 (s, 1H), 6.55 (s, 1H), 5.04-4.93 (m, 2H), 4.70-4.56 (m, 1H), 4.50-4.40 (m, 1H), 4.37-4.26 (m, 1H), 4.24-4.16 (m, 1H), 4.00-3.89 (m, 1H), 3.76-3.60 (m, 3H), 3.60-3.45 (m, 6H), 3.45-3.36 (m, 2H), 3.01-2.80 (m, 2H), 2.62-2.40 (m, 2H).

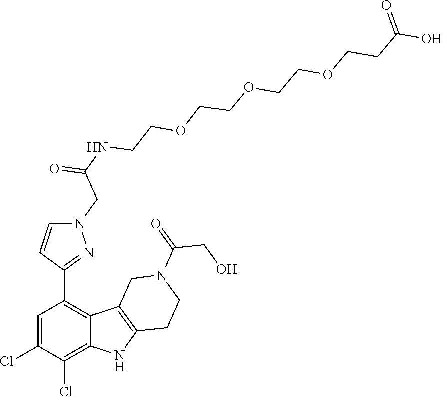 [SMILES: O=C(O)CCOCCOCCOCCNC(=O)Cn1ccc(-c2cc(Cl)c(Cl)c3[nH]c4c(c23)CN(C(=O)CO)CC4)n1]

Linker 97: 1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12-trioxa-3-azapentadecan-15-oic acid (XH181-155). Yellow solid, 71% yield. ¹H NMR (400 MHz, CD₃OD) δ 7.74 (s, 1H), 7.20 (s, 1H), 6.54 (s, 1H), 5.03-4.95 (m, 2H), 4.79-4.58 (m, 1H), 4.53-4.36 (m, 1H), 4.35-4.24 (m, 1H), 4.24-4.11 (m, 1H), 4.01-3.83 (m, 1H), 3.75-3.60 (m, 3H), 3.60-3.33 (m, 12H), 3.00-2.72 (m, 2H), 2.60-2.44 (m, 2H).

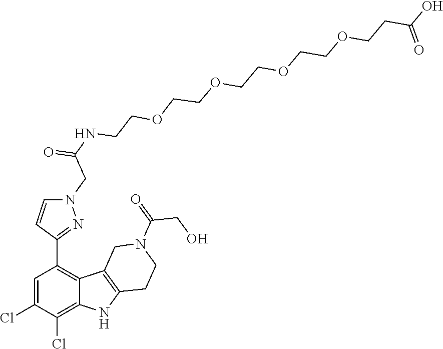 [SMILES: O=C(O)CCOCCOCCOCCOCCNC(=O)Cn1ccc(-c2cc(Cl)c(Cl)c3[nH]c4c(c23)CN(C(=O)CO)CC4)n1]

Linker 98: 1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12,15-tetraoxa-3-azaoctadecan-18-oic acid (XH181-156). Yellow solid, 61% yield. ¹H NMR (400 MHz, CD₃OD) δ 7.74 (s, 1H), 7.21 (s, 1H), 6.55 (s, 1H), 4.98-4.91 (m, 2H), 4.72-4.60 (m, 1H), 4.52-4.40 (m, 1H), 4.38-4.27 (m, 1H), 4.25-4.17 (m, 1H), 3.99-3.86 (m, 1H), 3.79-3.61 (m, 3H), 3.61-3.31 (m, 16H), 2.97-2.82 (m, 2H), 2.64-2.38 (m, 2H).

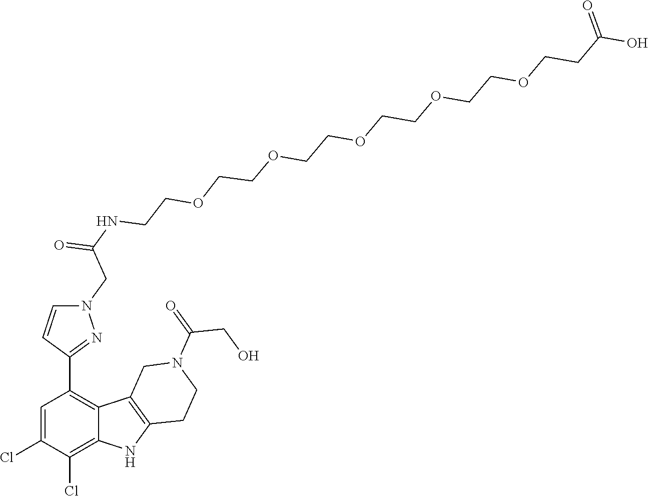 [SMILES: O=C(O)CCOCCOCCOCCOCCOCCNC(=O)Cn1ccc(-c2cc(Cl)c(Cl)c3[nH]c4c(c23)CN(C(=O)CO)CC4)n1]

Linker 99: 1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12,15,18-pentaoxa-3-azahenicosan-21-oic acid (XH181-157). Yellow solid, 63% yield. Yellow solid, 71%. ¹H NMR (400 MHz, CD₃OD) δ 7.75 (s, 1H), 7.21 (s, 1H), 6.55 (s, 1H), 5.01-4.93 (m, 2H), 4.70-4.61 (m, 1H), 4.52-4.42 (m, 1H), 4.38-4.26 (m, 1H), 4.26-4.13 (m, 1H), 4.00-3.85 (m, 1H), 3.78-3.64 (m, 3H), 3.62-3.35 (m, 20H), 2.98-2.77 (m, 2H), 2.65-2.37 (m, 2H).

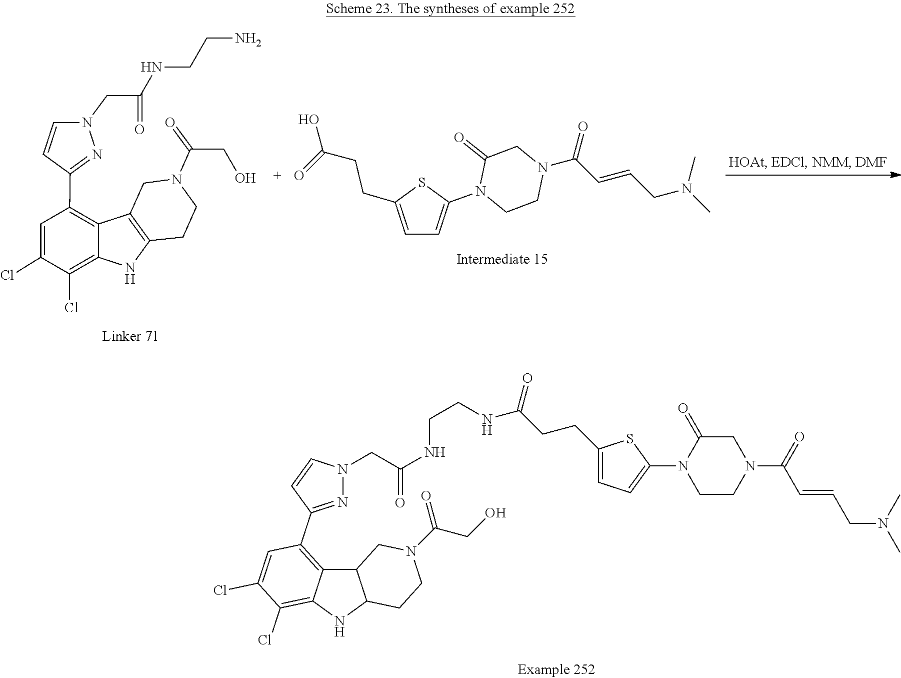 [SMILES: NCCNC(=O)Cn1ccc(-c2cc(Cl)c(Cl)c3[nH]c4c(c23)CN(C(=O)CO)CC4)n1 ; CN(C)C/C=C/C(=O)N1CCN(c2ccc(CCC(=O)O)s2)C(=O)C1 ; CN(C)C/C=C/C(=O)N1CCN(c2ccc(CCC(=O)NCCNC(=O)Cn3ccc(-c4cc(Cl)c(Cl)c5c4C4CN(C(=O)CO)CCC4N5)n3)s2)C(=O)C1]

Example 252: (E)-N-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethyl)-3-(5-(4-(4-(dimethylamino)but-2-enoyl)-2-oxopiperazin-1-yl)thiophen-2-yl)propenamide (ZD178-22). To a solution of Linker 71 (0.015 mmol, 1.0 eq) and Intermediate 15 (0.018 mmol, 1.2 eq) in dimethylformamide (1.0 mL) was added HOAt (0.03 mmol, 2.0 eq), NMM (0.15 mmol, 10.0 eq), and EDCI (0.03 mmol, 2.0 eq). The resulting reaction solution was stirred at rt for 12 h. Then the resulting residue was purified by reverse-phase chromatography to yield the desired product. Brown solid, 50% yield. ¹H NMR (400 MHz, CD₃OD) δ 7.83-7.71 (m, 1H), 7.34-7.18 (m, 1H), 7.04-6.69 (m, 2H), 6.60 (d, J=18.4 Hz, 3H), 5.05-4.92 (m, 2H), 4.71-4.17 (m, 6H), 4.08-3.65 (m, 8H), 3.65-3.43 (m, 4H), 3.09-2.81 (m, 10H), 2.63-2.41 (m, 2H). MS (ESI) [M+H]⁺=812.4.

Examples 253-265 were synthesized following the same procedure for preparing example 252 from related linkers and intermediate 15.

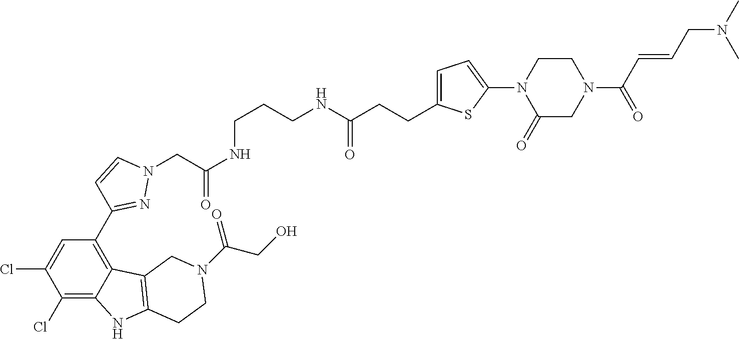 [SMILES: CN(C)C/C=C/C(=O)N1CCN(c2ccc(CCC(=O)NCCCNC(=O)Cn3ccc(-c4cc(Cl)c(Cl)c5[nH]c6c(c45)CN(C(=O)CO)CC6)n3)s2)C(=O)C1]

Example 253: (E)-N-(3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)propyl)-3-(5-(4-(4-(dimethylamino)but-2-enoyl)-2-oxopiperazin-1-yl)thiophen-2-yl)propenamide(ZD178-23) Brown solid, 50% yield. ¹H NMR (400 MHz, CD₃OD) δ 7.92-7.71 (m, 1H), 7.38-7.12 (m, 1H), 6.99-6.67 (m, 2H), 6.66-6.51 (m, 3H), 5.09-4.90 (m, 2H), 4.72-4.15 (m, 6H), 4.02-3.63 (m, 8H), 3.25-2.81 (m, 14H), 2.60-2.41 (m, 2H), 1.96-1.44 (m, 2H). MS (ESI) [M+H]⁺=826.1.

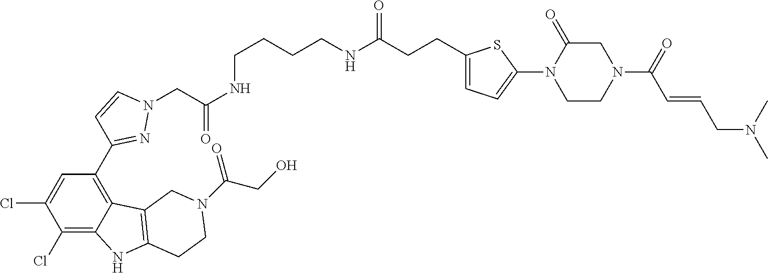 [SMILES: CN(C)C/C=C/C(=O)N1CCN(c2ccc(CCC(=O)NCCCCNC(=O)Cn3ccc(-c4cc(Cl)c(Cl)c5[nH]c6c(c45)CN(C(=O)CO)CC6)n3)s2)C(=O)C1]

Example 254: (E)-N-(4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)butyl)-3-(5-(4-(4-(dimethylamino)but-2-enoyl)-2-oxopiperazin-1-yl)thiophen-2-yl)propenamide (ZD178-24) Brown solid, 48% yield. ¹H NMR (400 MHz, CD₃OD) δ 7.82-7.72 (m, 1H), 7.40-7.17 (m, 1H), 7.01-6.64 (m, 2H), 6.68-6.50 (m, 3H), 4.98-4.90 (m, 2H), 4.72-4.14 (m, 6H), 4.03-3.63 (m, 8H), 3.25-2.84 (m, 14H), 2.60-2.36 (m, 2H), 1.93-1.27 (m, 4H). MS (ESI) [M+H]⁺=840.3.

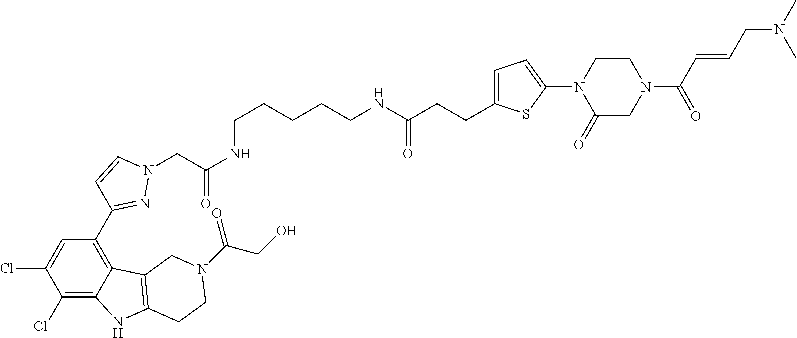 [SMILES: CN(C)C/C=C/C(=O)N1CCN(c2ccc(CCC(=O)NCCCCCNC(=O)Cn3ccc(-c4cc(Cl)c(Cl)c5[nH]c6c(c45)CN(C(=O)CO)CC6)n3)s2)C(=O)C1]

Example 255: (E)-N-(5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)pentyl)-3-(5-(4-(4-(dimethylamino)but-2-enoyl)-2-oxopiperazin-1-yl)thiophen-2-yl)propanamide (ZD178-25) Brown solid, 48% yield. ¹H NMR (400 MHz, CD₃OD) δ 7.95-7.69 (m, 1H), 7.48-7.12 (m, 1H), 7.03-6.67 (m, 2H), 6.71-6.44 (m, 3H), 5.05-4.90 (m, 2H), 4.74-4.15 (m, 6H), 4.06-3.65 (m, 8H), 3.25-2.74 (m, 14H), 2.57-2.39 (m, 2H), 1.64-1.13 (m, 6H). MS (ESI) [M+H]⁺=854.3.

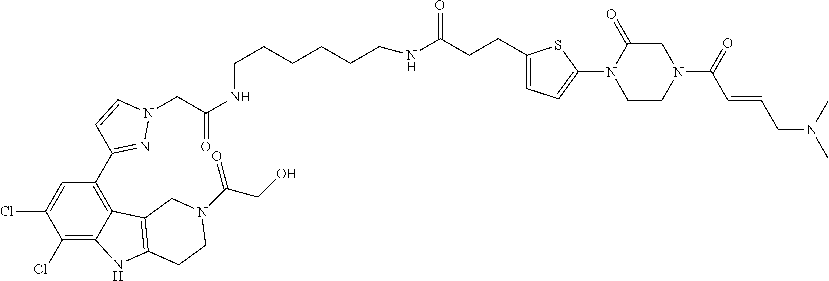 [SMILES: CN(C)C/C=C/C(=O)N1CCN(c2ccc(CCC(=O)NCCCCCCNC(=O)Cn3ccc(-c4cc(Cl)c(Cl)c5[nH]c6c(c45)CN(C(=O)CO)CC6)n3)s2)C(=O)C1]

Example 256: (E)-N-(6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)hexyl)-3-(5-(4-(4-(dimethylamino)but-2-enoyl)-2-oxopiperazin-1-yl)thiophen-2-yl)propenamide (ZD178-26) Brown solid, 52% yield. ¹H NMR (400 MHz, CD₃OD) δ 8.03-7.69 (m, 1H), 7.42-7.15 (m, 1H), 7.02-6.65 (m, 2H), 6.67-6.49 (m, 3H), 5.13-4.92 (m, 2H), 4.75-4.15 (m, 6H), 4.06-3.57 (m, 8H), 3.26-2.69 (m, 14H), 2.60-2.26 (m, 2H), 1.74-0.96 (m, 8H). MS (ESI) [M+H]⁺=868.4.

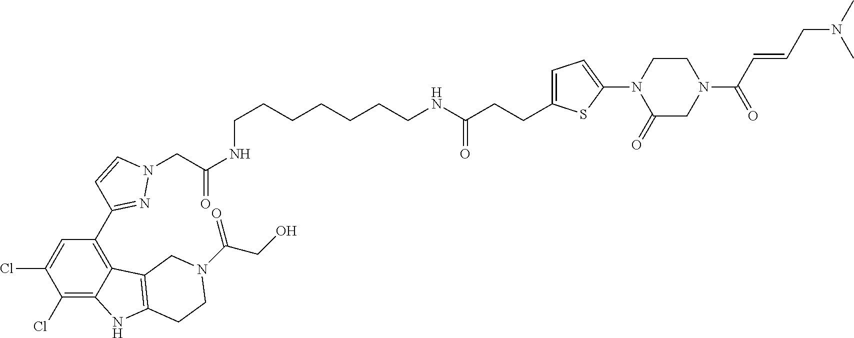 [SMILES: CN(C)C/C=C/C(=O)N1CCN(c2ccc(CCC(=O)NCCCCCCCNC(=O)Cn3ccc(-c4cc(Cl)c(Cl)c5[nH]c6c(c45)CN(C(=O)CO)CC6)n3)s2)C(=O)C1]

Example 257 (E)-N-(7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)heptyl)-3-(5-(4-(4-(dimethylamino)but-2-enoyl)-2-oxopiperazin-1-yl)thiophen-2-yl)propenamide (ZD178-27) Brown solid, 52% yield. ¹H NMR (400 MHz, CD₃OD) δ 7.89-7.67 (m, 1H), 7.38-7.12 (m, 1H), 7.07-6.67 (m, 2H), 6.73-6.44 (m, 3H), 5.30-4.90 (m, 2H), 4.77-4.17 (m, 6H), 4.10-3.61 (m, 8H), 3.27-2.82 (m, 14H), 2.63-2.35 (m, 2H), 1.62-1.05 (m, 10H). MS (ESI) [M+H]⁺=882.6.

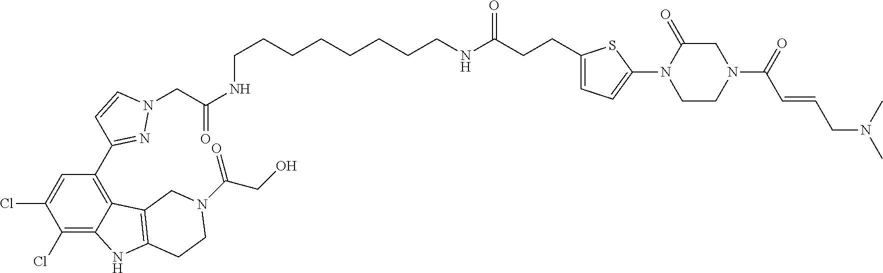 [SMILES: CN(C)C/C=C/C(=O)N1CCN(c2ccc(CCC(=O)NCCCCCCCCNC(=O)Cn3ccc(-c4cc(Cl)c(Cl)c5[nH]c6c(c45)CN(C(=O)CO)CC6)n3)s2)C(=O)C1]

Example 258: (E)-N-(8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)octyl)-3-(5-(4-(4-(dimethylamino)but-2-enoyl)-2-oxopiperazin-1-yl)thiophen-2-yl)propenamide (ZD178-28) Brown solid, 45% yield. ¹H NMR (400 MHz, CD₃OD) δ 7.87-7.59 (m, 1H), 7.42-7.10 (m, 1H), 7.03-6.69 (m, 2H), 6.69-6.48 (m, 3H), 5.07-4.90 (m, 2H), 4.74-4.17 (m, 6H), 4.14-3.57 (m, 8H), 3.27-2.71 (m, 14H), 2.59-2.33 (m, 2H), 1.71-0.92 (m, 12H). MS (ESI) [M+H]⁺=896.3.

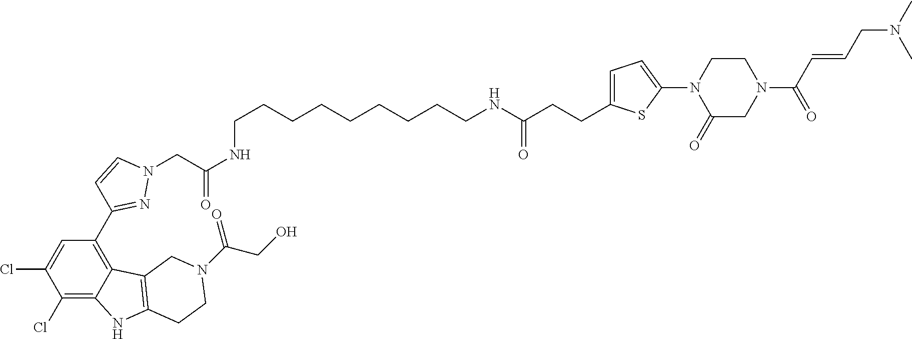 [SMILES: CN(C)C/C=C/C(=O)N1CCN(c2ccc(CCC(=O)NCCCCCCCCCNC(=O)Cn3ccc(-c4cc(Cl)c(Cl)c5[nH]c6c(c45)CN(C(=O)CO)CC6)n3)s2)C(=O)C1]

Example 259 (E)-N-(9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)nonyl)-3-(5-(4-(4-(dimethylamino)but-2-enoyl)-2-oxopiperazin-1-yl)thiophen-2-yl)propenamide (ZD178-29, MS7829) Brown solid, 45% yield. ¹H NMR (400 MHz, CD₃OD) δ 7.89-7.61 (m, 1H), 7.33-7.13 (m, 1H), 7.03-6.67 (m, 2H), 6.67-6.51 (m, 3H), 4.98-4.91 (m, 2H), 4.70-4.65 (m, 1H), 4.50-4.41 (m, 2H), 4.41-4.31 (m, 2H), 4.25-4.20 (m, 1H), 4.07-3.60 (m, 8H), 3.27-3.17 (m, 2H), 3.15-3.07 (m, 2H), 3.07-3.00 (m, 2H), 2.95-2.84 (m, 8H), 2.60-2.40 (m, 2H), 1.70-0.99 (m, 14H). MS (ESI) [M+H]⁺=910.4.

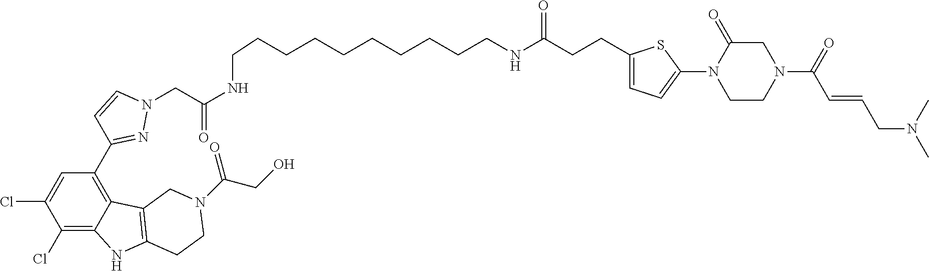 [SMILES: CN(C)C/C=C/C(=O)N1CCN(c2ccc(CCC(=O)NCCCCCCCCCCNC(=O)Cn3ccc(-c4cc(Cl)c(Cl)c5[nH]c6c(c45)CN(C(=O)CO)CC6)n3)s2)C(=O)C1]

Example 260: (E)-N-(10-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)decyl)-3-(5-(4-(4-(dimethylamino)but-2-enoyl)-2-oxopiperazin-1-yl)thiophen-2-yl)propenamide (ZD178-30) Brown solid, 47% yield. ¹H NMR (400 MHz, CD₃OD) δ 8.00-7.57 (m, 1H), 7.34-7.14 (m, 1H), 7.05-6.69 (m, 2H), 6.71-6.44 (m, 3H), 5.02-4.92 (m, 2H), 4.75-4.17 (m, 6H), 4.10-3.63 (m, 8H), 3.27-2.80 (m, 14H), 2.65-2.42 (m, 2H), 1.77-0.96 (m, 16H). MS (ESI) [M+H]⁺=924.5.

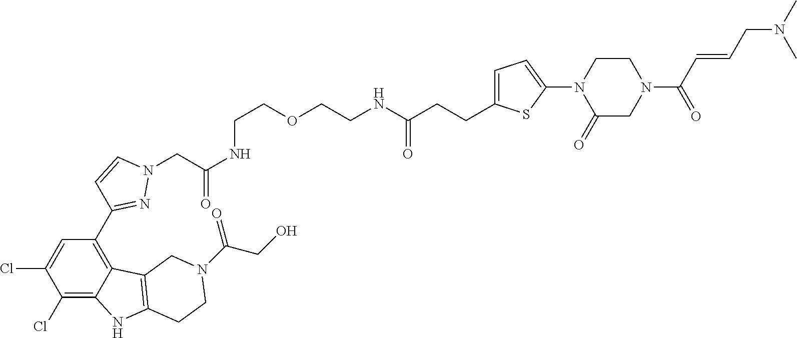 [SMILES: CN(C)C/C=C/C(=O)N1CCN(c2ccc(CCC(=O)NCCOCCNC(=O)Cn3ccc(-c4cc(Cl)c(Cl)c5[nH]c6c(c45)CN(C(=O)CO)CC6)n3)s2)C(=O)C1]

Example 261: (E)-N-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethyl)-3-(5-(4-(4-(dimethylamino)but-2-enoyl)-2-oxopiperazin-1-yl)thiophen-2-yl)propenamide (ZD178-31) Brown solid, 47% yield. ¹H NMR (400 MHz, CD₃OD) δ 7.85-7.68 (m, 1H), 7.48-7.14 (m, 1H), 7.05-6.65 (m, 2H), 6.67-6.40 (m, 3H), 5.09-4.90 (m, 2H), 4.77-4.17 (m, 6H), 4.10-3.63 (m, 8H), 3.61-3.36 (m, 4H), 3.27-3.23 (m, 4H), 3.12-2.71 (m, 10H), 2.59-2.33 (m, 2H). MS (ESI) [M+H]⁺=856.4

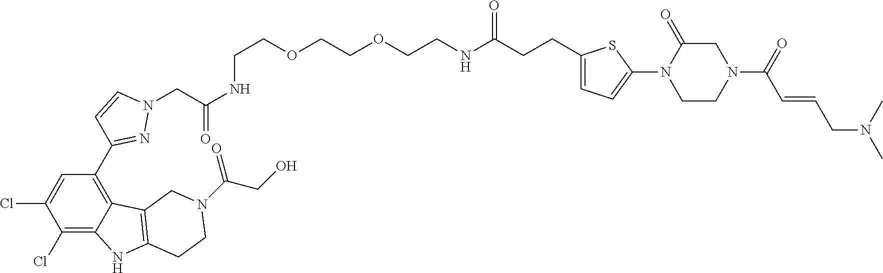 [SMILES: CN(C)C/C=C/C(=O)N1CCN(c2ccc(CCC(=O)NCCOCCOCCNC(=O)Cn3ccc(-c4cc(Cl)c(Cl)c5[nH]c6c(c45)CN(C(=O)CO)CC6)n3)s2)C(=O)C1]

Example 262: (E)-N-(2-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethoxy)ethyl)-3-(5-(4-(4-(dimethylamino)but-2-enoyl)-2-oxopiperazin-1-yl)thiophen-2-yl)propenamide (ZD178-32) Brown solid, 53% yield. ¹H NMR (400 MHz, CD₃OD) δ 7.89-7.70 (m, 1H), 7.30-7.14 (m, 1H), 7.00-6.69 (m, 2H), 6.67-6.49 (m, 3H), 5.10-4.92 (m, 2H), 4.72-4.15 (m, 6H), 4.05-3.65 (m, 8H), 3.62-3.35 (m, 10H), 3.29-3.24 (m, 2H), 3.10-2.83 (m, 10H), 2.57-2.40 (m, 2H). MS (ESI) [M+H]⁺=900.2.

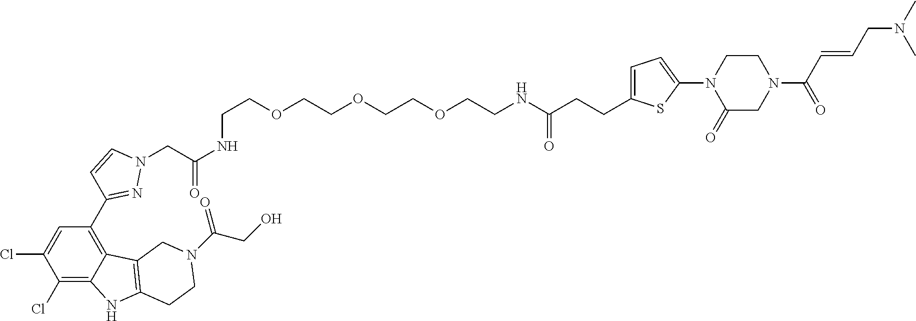 [SMILES: CN(C)C/C=C/C(=O)N1CCN(c2ccc(CCC(=O)NCCOCCOCCOCCNC(=O)Cn3ccc(-c4cc(Cl)c(Cl)c5[nH]c6c(c45)CN(C(=O)CO)CC6)n3)s2)C(=O)C1]

Example 263 (E)-N-(l-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12-trioxa-3-azatetradecan-14-yl)-3-(5-(4-(4-(dimethylamino)but-2-enoyl)-2-oxopiperazin-1-yl)thiophen-2-yl)propenamide (ZD178-33) Brown solid, 53% yield. ¹H NMR (400 MHz, CD₃OD) δ 8.08-7.65 (m, 1H), 7.36-7.12 (m, 1H), 7.03-6.69 (m, 2H), 6.68-6.50 (m, 3H), 5.12-4.91 (m, 2H), 4.76-4.13 (m, 6H), 4.13-3.68 (m, 8H), 3.63-3.35 (m, 16H), 3.07-2.80 (m, 10H), 2.59-2.40 (m, 2H). MS (ESI) [M+H]⁺=944.1.

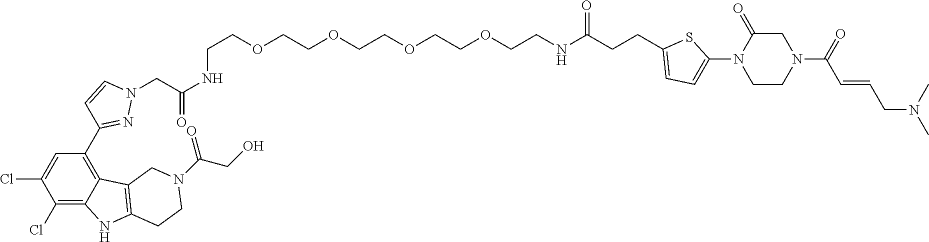 [SMILES: CN(C)C/C=C/C(=O)N1CCN(c2ccc(CCC(=O)NCCOCCOCCOCCOCCNC(=O)Cn3ccc(-c4cc(Cl)c(Cl)c5[nH]c6c(c45)CN(C(=O)CO)CC6)n3)s2)C(=O)C1]

Example 264 (E)-N-(1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12,15-tetraoxa-3-azaheptadecan-17-yl)-3-(5-(4-(4-(dimethylamino)but-2-enoyl)-2-oxopiperazin-1-yl)thiophen-2-yl)propenamide (ZD178-34) Brown solid, 51% yield. ¹H NMR (400 MHz, CD₃OD) δ 7.92-7.57 (m, 1H), 7.37-7.09 (m, 1H), 7.03-6.68 (m, 2H), 6.68-6.52 (m, 3H), 5.10-4.93 (m, 2H), 4.73-4.16 (m, 6H), 4.11-3.64 (m, 8H), 3.62-3.37 (m, 20H), 3.21-2.81 (m, 10H), 2.62-2.30 (m, 2H). MS (ESI) [M+H]⁺=988.3.

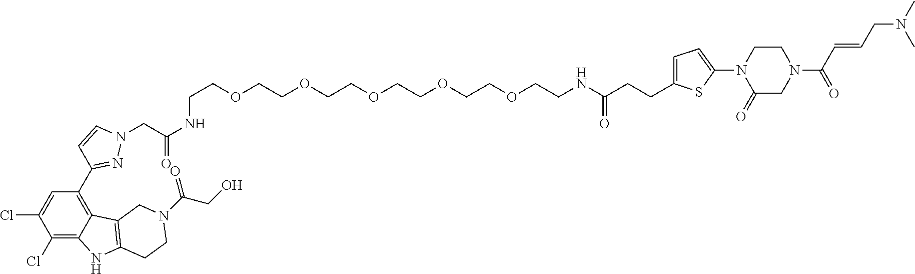 [SMILES: CN(C)C/C=C/C(=O)N1CCN(c2ccc(CCC(=O)NCCOCCOCCOCCOCCOCCNC(=O)Cn3ccc(-c4cc(Cl)c(Cl)c5[nH]c6c(c45)CN(C(=O)CO)CC6)n3)s2)C(=O)C1]

Example 265: (E)-N-(1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12,15,18-pentaoxa-3-azaicosan-20-yl)-3-(5-(4-(4-(dimethylamino)but-2-enoyl)-2-oxopiperazin-1-yl)thiophen-2-yl)propenamide (ZD178-35) Brown solid, 51% yield. ¹H NMR (400 MHz, CD₃OD) δ 7.91-7.58 (m, 1H), 7.37-7.07 (m, 1H), 7.05-6.70 (m, 2H), 6.72-6.47 (m, 3H), 5.16-4.93 (m, 2H), 4.78-4.14 (m, 6H), 4.10-3.66 (m, 8H), 3.61-3.36 (m, 24H), 3.18-2.82 (m, 10H), 2.70-2.40 (m, 2H). MS (ESI) [M+H]⁺=1032.3.

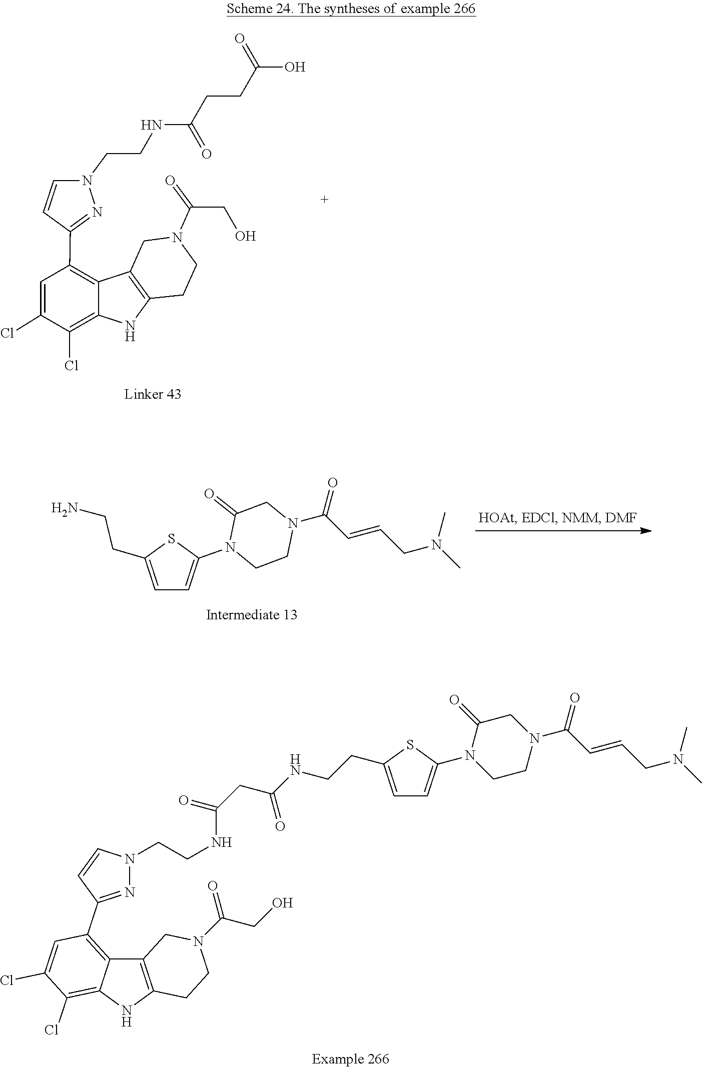 [SMILES: O=C(O)CCC(=O)NCCn1ccc(-c2cc(Cl)c(Cl)c3[nH]c4c(c23)CN(C(=O)CO)CC4)n1 ; CN(C)C/C=C/C(=O)N1CCN(c2ccc(CCN)s2)C(=O)C1 ; CN(C)C/C=C/C(=O)N1CCN(c2ccc(CCNC(=O)CC(=O)NCCn3ccc(-c4cc(Cl)c(Cl)c5[nH]c6c(c45)CN(C(=O)CO)CC6)n3)s2)C(=O)C1]

Example 266 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N4-(2-(5-(4-(4-(dimethylamino)but-2-enoyl)-2-oxopiperazin-1-yl)thiophen-2-yl)ethyl)succinamide (ZD178-58-1) To a solution of Linker 43 (0.015 mmol, 1.0 eq) and Intermediate 13 (0.018 mmol, 1.2 eq) in dimethylformamide (1.0 mL) was added HOAt (0.03 mmol, 2.0 eq), NMM (0.15 mmol, 10.0 eq), and EDCI (0.03 mmol, 2.0 eq). The resulting reaction solution was stirred at rt for 12 h. Then the resulting residue was purified by reverse-phase chromatography to yield the desired product. Brown solid, 55% yield. ¹H NMR (400 MHz, CD₃OD) δ 7.81-7.62 (m, 1H), 7.30-7.11 (m, 1H), 7.03-6.69 (m, 2H), 6.69-6.39 (m, 3H), 4.64-4.11 (m, 8H), 4.08-3.59 (m, 10H), 3.44-3.34 (m, 2H), 3.22-3.14 (m, 2H), 3.02-2.84 (m, 8H), 2.58-2.34 (m, 4H). MS (ESI) [M+H]⁺=826.3.

Example 267-279 were synthesized following the same procedure for preparing example 266 from related linkers and intermediate 13.

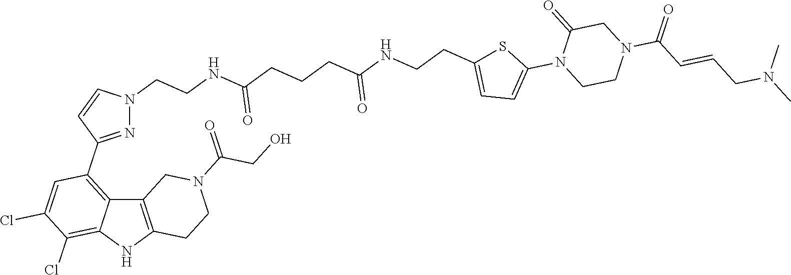 [SMILES: CN(C)C/C=C/C(=O)N1CCN(c2ccc(CCNC(=O)CCCC(=O)NCCn3ccc(-c4cc(Cl)c(Cl)c5[nH]c6c(c45)CN(C(=O)CO)CC6)n3)s2)C(=O)C1]

Example 267 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N5-(2-(5-(4-(4-(dimethylamino)but-2-enoyl)-2-oxopiperazin-1-yl)thiophen-2-yl)ethyl)glutaramide (ZD178-58-2) Brown solid, 55% yield. ¹H NIR (400 MHz, CD₃OD) δ 7.81-7.60 (m, 1H), 7.35-7.11 (m, 1H), 7.05-6.72 (m, 2H), 6.68-6.32 (m, 3H), 4.67-4.08 (m, 8H), 4.12-3.58 (m, 10H), 3.23-3.10 (m, 2H), 3.02-2.72 (m, 10H), 2.47-2.05 (m, 4H), 2.01-1.64 (m, 2H). MS (ESI) [M+H]⁺=840.2.

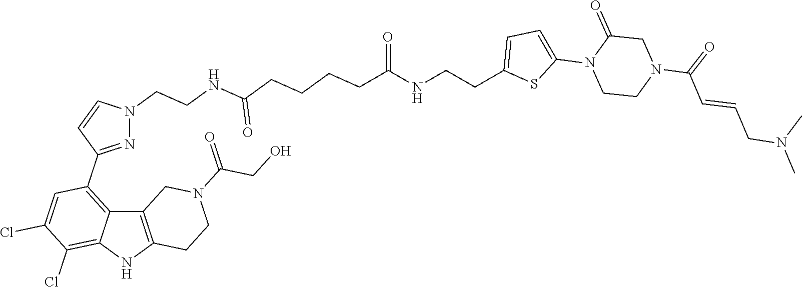 [SMILES: CN(C)C/C=C/C(=O)N1CCN(c2ccc(CCNC(=O)CCCCC(=O)NCCn3ccc(-c4cc(Cl)c(Cl)c5[nH]c6c(c45)CN(C(=O)CO)CC6)n3)s2)C(=O)C1]

Example 268 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N6-(2-(5-(4-(4-(dimethylamino)but-2-enoyl)-2-oxopiperazin-1-yl)thiophen-2-yl)ethyl)adipamide (ZD178-58-3) Brown solid, 49% yield. ¹H NMR (400 MHz, CD₃OD) δ 7.89-7.55 (m, 1H), 7.41-7.09 (m, 1H), 7.07-6.68 (m, 2H), 6.72-6.32 (m, 3H), 4.62-4.09 (m, 8H), 4.09-3.63 (m, 10H), 3.24-3.09 (m, 2H), 3.03-2.74 (m, 10H), 2.30-2.05 (m, 4H), 1.68-1.30 (m, 4H). MS (ESI) [M+H]⁻=854.9.

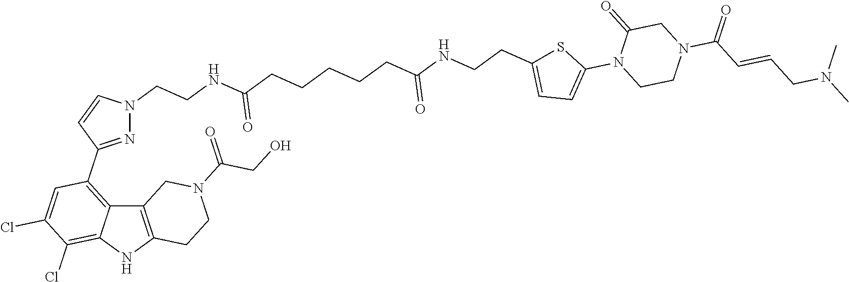 [SMILES: CN(C)C/C=C/C(=O)N1CCN(c2ccc(CCNC(=O)CCCCCC(=O)NCCn3ccc(-c4cc(Cl)c(Cl)c5[nH]c6c(c45)CN(C(=O)CO)CC6)n3)s2)C(=O)C1]

Example 269 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N7-(2-(5-(4-(4-(dimethylamino)but-2-enoyl)-2-oxopiperazin-1-yl)thiophen-2-yl)ethyl)heptanediamide (ZD178-58-4) Brown solid, 48% yield. ¹H NMR (400 MHz, CD₃OD) δ 7.81-7.57 (m, 1H), 7.32-7.14 (m, 1H), 7.09-6.70 (m, 2H), 6.72-6.32 (m, 3H), 4.69-4.10 (m, 8H), 4.10-3.62 (m, 10H), 3.44-3.34 (m, 2H), 3.21-2.80 (m, 10H), 2.28-2.04 (m, 4H), 1.71-1.09 (m, 6H). MS (ESI) [M+H]⁺=868.3.

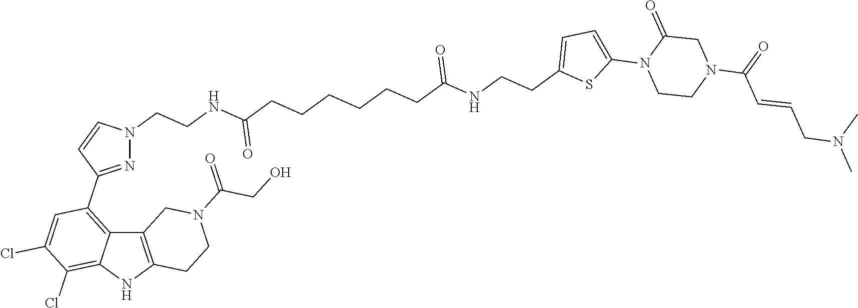 [SMILES: CN(C)C/C=C/C(=O)N1CCN(c2ccc(CCNC(=O)CCCCCCC(=O)NCCn3ccc(-c4cc(Cl)c(Cl)c5[nH]c6c(c45)CN(C(=O)CO)CC6)n3)s2)C(=O)C1]

Example 270: (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N8-(2-(5-(4-(4-(dimethylamino)but-2-enoyl)-2-oxopiperazin-1-yl)thiophen-2-yl)ethyl)octanediamide (ZD178-58-5) Brown solid, 50% yield. ¹H NMR (400 MHz, CD₃OD) δ 7.79-7.51 (m, 1H), 7.37-7.12 (m, 1H), 7.07-6.72 (m, 2H), 6.72-6.30 (m, 3H), 4.71-4.10 (m, 8H), 4.13-3.58 (m, 10H), 3.46-3.35 (m, 2H), 3.23-2.74 (m, 10H), 2.37-2.03 (m, 4H), 1.66-0.90 (m, 8H).MS (ESI) [M+H]⁺=882.5.

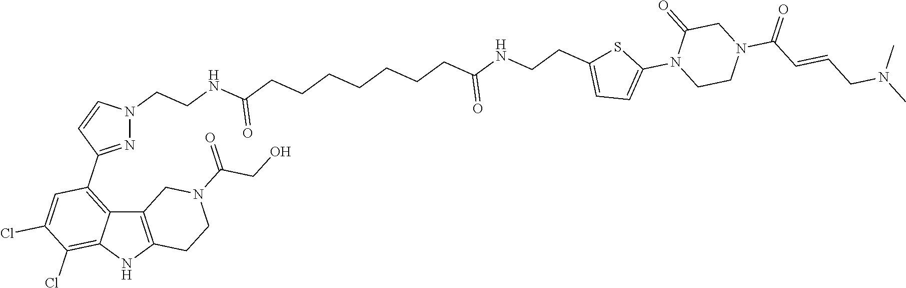 [SMILES: CN(C)C/C=C/C(=O)N1CCN(c2ccc(CCNC(=O)CCCCCCCC(=O)NCCn3ccc(-c4cc(Cl)c(Cl)c5[nH]c6c(c45)CN(C(=O)CO)CC6)n3)s2)C(=O)C1]

Example 271: (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N9-(2-(5-(4-(4-(dimethylamino)but-2-enoyl)-2-oxopiperazin-1-yl)thiophen-2-yl)ethyl)nonanediamide (ZD178-58-6) Brown solid, 48% yield. ¹H NMR (400 MHz, CD₃OD) δ 7.83-7.56 (m, 1H), 7.33-7.10 (m, 1H), 7.04-6.71 (m, 2H), 6.75-6.29 (m, 3H), 4.68-4.11 (m, 8H), 4.13-3.58 (m, 10H), 3.45-3.35 (m, 2H), 3.23-2.74 (m, 10H), 2.29-2.00 (m, 4H), 1.68-1.12 (m, 10H). MS (ESI) [M+H]⁺=896.4.

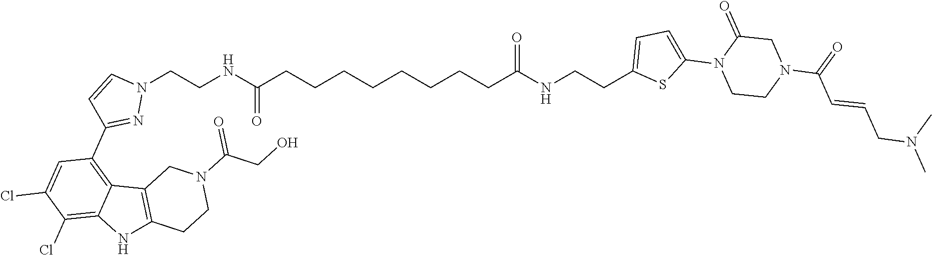 [SMILES: CN(C)C/C=C/C(=O)N1CCN(c2ccc(CCNC(=O)CCCCCCCCC(=O)NCCn3ccc(-c4cc(Cl)c(Cl)c5[nH]c6c(c45)CN(C(=O)CO)CC6)n3)s2)C(=O)C1]

Example 272 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N10-(2-(5-(4-(4-(dimethylamino)but-2-enoyl)-2-oxopiperazin-1-yl)thiophen-2-yl)ethyl)decanediamide (ZD178-58-7) Brown solid, 51% yield. ¹H NMR (400 MHz, CD₃OD) δ 7.91-7.58 (m, 1H), 7.37-7.14 (m, 1H), 7.08-6.71 (m, 2H), 6.73-6.36 (m, 3H), 4.70-4.11 (m, 8H), 4.14-3.54 (m, 10H), 3.49-3.35 (m, 2H), 3.27-2.70 (m, 10H), 2.31-1.86 (m, 4H), 1.62-1.10 (m, 12H). MS (ESI) [M+H]⁺=910.5.

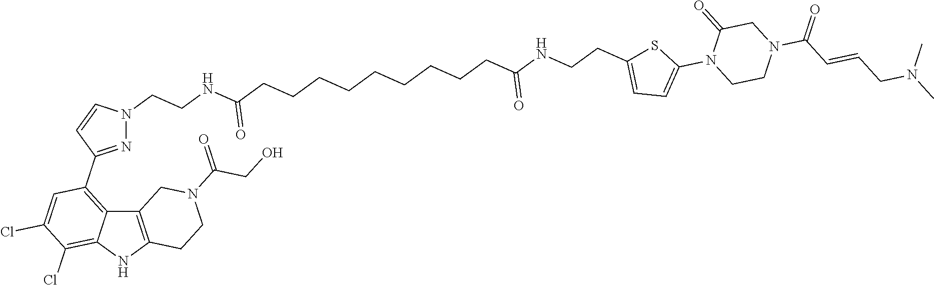 [SMILES: CN(C)C/C=C/C(=O)N1CCN(c2ccc(CCNC(=O)CCCCCCCCCC(=O)NCCn3ccc(-c4cc(Cl)c(Cl)c5[nH]c6c(c45)CN(C(=O)CO)CC6)n3)s2)C(=O)C1]

Example 273 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N11-(2-(5-(4-(4-(dimethylamino)but-2-enoyl)-2-oxopiperazin-1-yl)thiophen-2-yl)ethyl)undecanediamide (ZD178-58-8, MS8588) Brown solid, 5100 yield. ¹H NMR (400 MHz, CD₃OD) δ 7.73-7.57 (m, 1H), 7.27-7.11 (m, 1H), 7.04-6.69 (m, 2H), 6.66-6.24 (m, 3H), 4.71-4.20 (m, 8H), 4.07-3.57 (in, 10H), 3.49-3.35 (m, 2H), 3.07-2.80 (m, 10H), 2.26-2.02 (m, 4H), 1.62-1.06 (m, 14H). MS (ESI) [M+H]⁺=924.3.

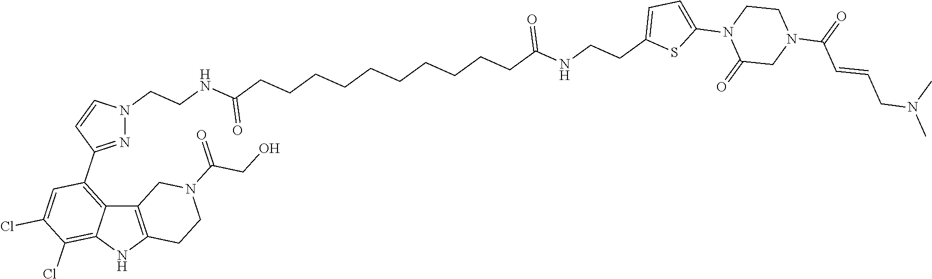 [SMILES: CN(C)C/C=C/C(=O)N1CCN(c2ccc(CCNC(=O)CCCCCCCCCCC(=O)NCCn3ccc(-c4cc(Cl)c(Cl)c5[nH]c6c(c45)CN(C(=O)CO)CC6)n3)s2)C(=O)C1]

Example 274 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N12-(2-(5-(4-(4-(dimethylamino)but-2-enoyl)-2-oxopiperazin-1-yl)thiophen-2-yl)ethyl)dodecanediamide (ZD178-58-9) Brown solid, 46% yield. ¹H NMR (400 MHz, CD₃OD) δ 7.81-7.56 (m, 1H), 7.33-7.14 (m, 1H), 7.06-6.75 (m, 2H), 6.75-6.35 (m, 3H), 4.75-4.10 (m, 8H), 4.08-3.61 (m, 10H), 3.54-3.36 (m, 2H), 3.23-2.71 (m, 10H), 2.38-2.04 (m, 4H), 1.73-0.95 (m, 16H). MS (ESI) [M+H]⁺=938.1.

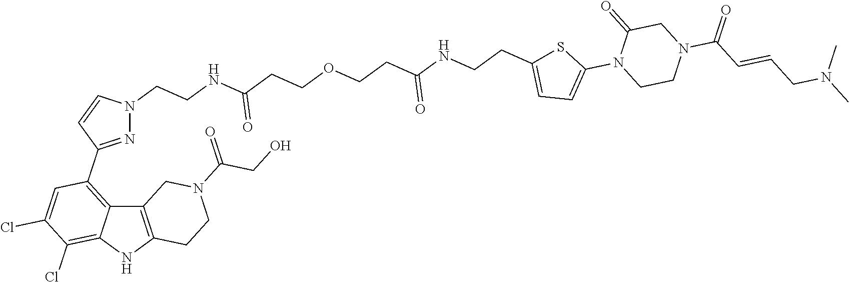 [SMILES: CN(C)C/C=C/C(=O)N1CCN(c2ccc(CCNC(=O)CCOCCC(=O)NCCn3ccc(-c4cc(Cl)c(Cl)c5[nH]c6c(c45)CN(C(=O)CO)CC6)n3)s2)C(=O)C1]

Example 275: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(3-((2-(5-(4-(4-(dimethylamino)but-2-enoyl)-2-oxopiperazin-1-yl)thiophen-2-yl)ethyl)amino)-3-oxopropoxy)propenamide (ZD178-58-10) Brown solid, 46% yield. ¹H NMR (400 MHz, CD₃OD) δ 7.87-7.57 (m, 1H), 7.32-7.10 (m, 1H), 7.06-6.71 (m, 2H), 6.66-6.39 (m, 3H), 4.69-4.09 (m, 8H), 4.07-3.55 (m, 14H), 3.41-3.35 (m, 2H), 3.20-2.78 (m, 10H), 2.49-2.29 (m, 4H). MS (ESI) [M+H]⁺=870.3.

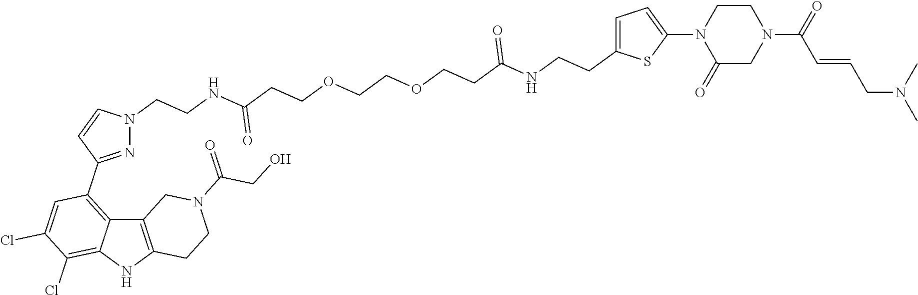 [SMILES: CN(C)C/C=C/C(=O)N1CCN(c2ccc(CCNC(=O)CCOCCOCCC(=O)NCCn3ccc(-c4cc(Cl)c(Cl)c5[nH]c6c(c45)CN(C(=O)CO)CC6)n3)s2)C(=O)C1]

Example 276: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(3-((2-(5-(4-(4-(dimethylamino)but-2-enoyl)-2-oxopiperazin-1-yl)thiophen-2-yl)ethyl)amino)-3-oxopropoxy)ethoxy)propenamide (ZD178-58-11) Brown solid, 53% yield. ¹H NMR (400 MHz, CD₃OD) δ 7.82-7.55 (m, 1H), 7.34-7.14 (m, 1H), 7.03-6.69 (m, 2H), 6.69-6.31 (m, 3H), 4.69-4.09 (m, 8H), 4.05-3.59 (m, 14H), 3.56-3.45 (m, 4H), 3.40-3.34 (m, 2H), 3.24-2.77 (m, 10H), 2.53-2.25 (m, 4H).MS (ESI) [M+H]⁺=914.3.

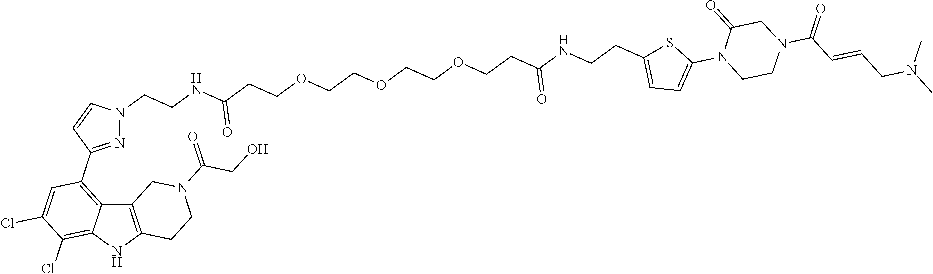 [SMILES: CN(C)C/C=C/C(=O)N1CCN(c2ccc(CCNC(=O)CCOCCOCCOCCC(=O)NCCn3ccc(-c4cc(Cl)c(Cl)c5[nH]c6c(c45)CN(C(=O)CO)CC6)n3)s2)C(=O)C1]

Example 277 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(2-(3-((2-(5-(4-(4-(dimethylamino)but-2-enoyl)-2-oxopiperazin-1-yl)thiophen-2-yl)ethyl)amino)-3-oxopropoxy)ethoxy)ethoxy)propenamide (ZD178-58-12) Brown solid, 53% yield. ¹H NMR (400 MHz, CD₃OD) δ 7.95-7.61 (m, 1H), 7.36-7.12 (m, 1H), 7.08-6.71 (m, 2H), 6.71-6.33 (m, 3H), 4.73-4.11 (m, 8H), 4.06-3.60 (m, 14H), 3.58-3.46 (m, 8H), 3.40-3.34 (m, 2H), 3.23-2.82 (m, 10H), 2.52-2.33 (m, 4H). MS (ESI) [M+H]⁺=958.4.

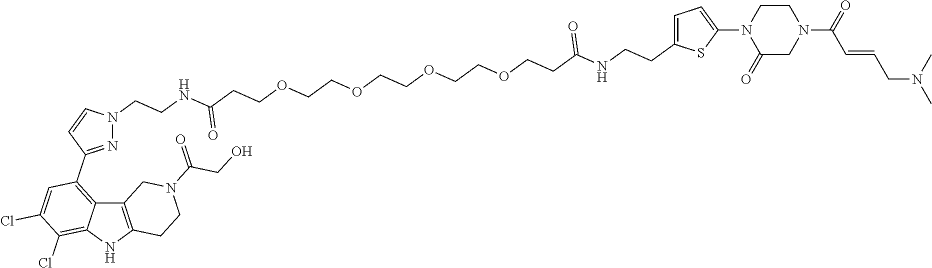 [SMILES: CN(C)C/C=C/C(=O)N1CCN(c2ccc(CCNC(=O)CCOCCOCCOCCOCCC(=O)NCCn3ccc(-c4cc(Cl)c(Cl)c5[nH]c6c(c45)CN(C(=O)CO)CC6)n3)s2)C(=O)C1]

Example 278 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N16-(2-(5-(4-(4-(dimethylamino)but-2-enoyl)-2-oxopiperazin-1-yl)thiophen-2-yl)ethyl)-4,7,10,13-tetraoxahexadecanediamide (ZD178-58-13) Brown solid, 57% yield. ¹H NMR (400 MHz, CD₃OD) δ 7.80-7.65 (m, 1H), 7.31-7.15 (m, 1H), 7.06-6.70 (m, 2H), 6.69-6.42 (m, 3H), 4.68-4.11 (m, 8H), 4.09-3.59 (m, 14H), 3.60-3.46 (m, 12H), 3.44-3.35 (m, 2H), 3.21-2.75 (m, 10H), 2.53-2.27 (m, 4H). MS (ESI) [M+H]⁺=1002.4.

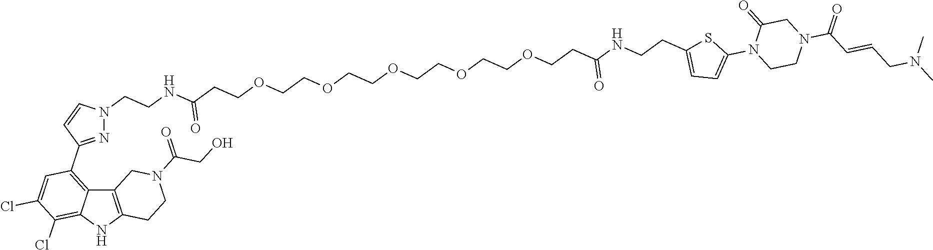 [SMILES: CN(C)C/C=C/C(=O)N1CCN(c2ccc(CCNC(=O)CCOCCOCCOCCOCCOCCC(=O)NCCn3ccc(-c4cc(Cl)c(Cl)c5[nH]c6c(c45)CN(C(=O)CO)CC6)n3)s2)C(=O)C1]

Example 279 (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N19-(2-(5-(4-(4-(dimethylamino)but-2-enoyl)-2-oxopiperazin-1-yl)thiophen-2-yl)ethyl)-4,7,10,13,16-pentaoxanonadecanediamide (ZD178-58-14) Brown solid, 50% yield. ¹H NMR (400 MHz, CD₃OD) δ 7.79-7.54 (m, 1H), 7.32-7.12 (m, 1H), 7.05-6.71 (m, 2H), 6.70-6.41 (m, 3H), 4.68-4.10 (m, 8H), 4.07-3.62 (m, 14H), 3.61-3.46 (m, 16H), 3.44-3.36 (m, 2H), 3.23-2.79 (m, 10H), 2.60-2.27 (m, 4H). MS (ESI) [M+H]⁺=1046.5.

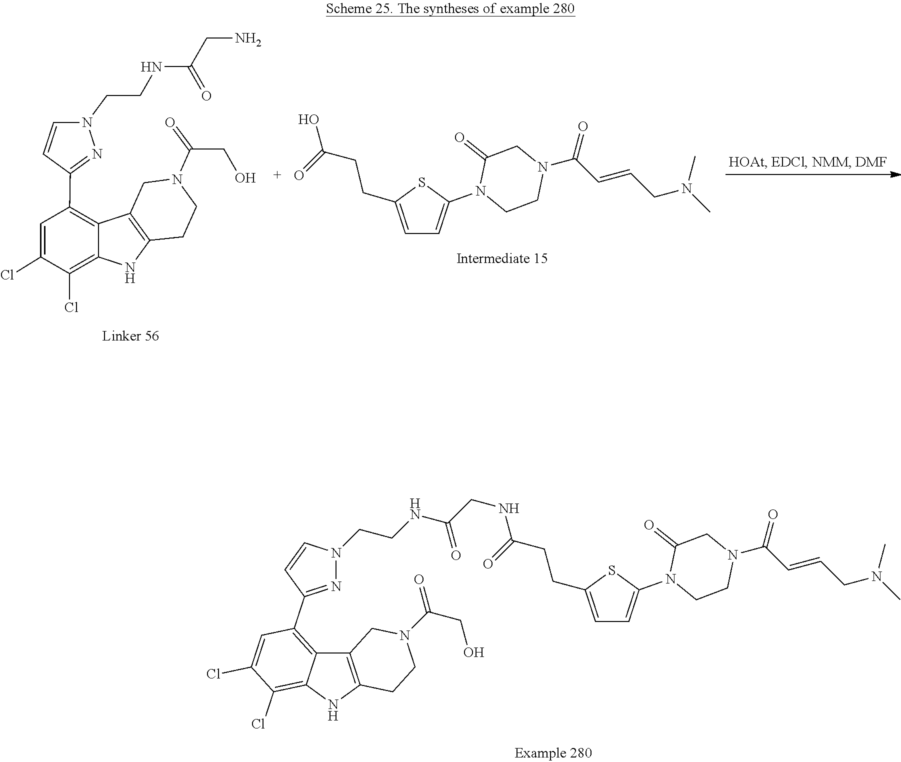 [SMILES: NCC(=O)NCCn1ccc(-c2cc(Cl)c(Cl)c3[nH]c4c(c23)CN(C(=O)CO)CC4)n1 ; CN(C)C/C=C/C(=O)N1CCN(c2ccc(CCC(=O)O)s2)C(=O)C1 ; CN(C)C/C=C/C(=O)N1CCN(c2ccc(CCC(=O)NCC(=O)NCCn3ccc(-c4cc(Cl)c(Cl)c5[nH]c6c(c45)CN(C(=O)CO)CC6)n3)s2)C(=O)C1]

Example 280. (E)-N-(2-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-2-oxoethyl)-3-(5-(4-(4-(dimethylamino)but-2-enoyl)-2-oxopiperazin-1-yl)thiophen-2-yl)propenamide (ZD178-63-1) To a solution of Linker 56 (0.015 mmol, 1.0 eq) and Intermediate 15 (0.018 mmol, 1.2 eq) in dimethylformamide (1.0 mL) was added HOAt (0.03 mmol, 2.0 eq), NMM (0.15 mmol, 10.0 eq), and EDCI (0.03 mmol, 2.0 eq). The resulting reaction solution was stirred at rt for 12 h. Then the resulting residue was purified by reverse-phase chromatography to yield the desired product.. Brown solid, 54% yield. ¹H NMR (400 MHz, CD₃OD) δ 7.73-7.59 (m, 1H), 7.25-7.15 (m, 1H), 7.01-6.69 (m, 2H), 6.64-6.40 (m, 3H), 4.60-4.17 (m, 8H), 4.05-3.59 (m, 12H), 3.07-2.81 (m, 10H), 2.65-2.36 (m, 2H). MS (ESI) [M+H]⁺=812.4.

Example 281-294 were synthesized following the same procedure for preparing example 280 from related linkers and intermediate 15.

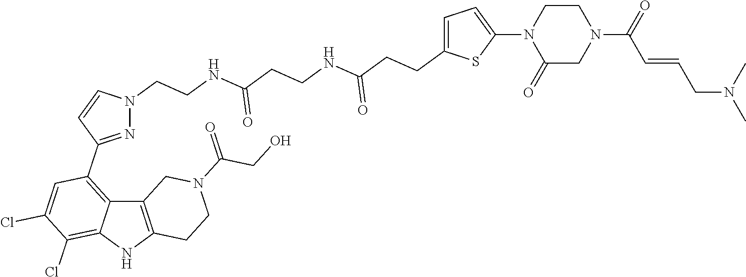 [SMILES: CN(C)C/C=C/C(=O)N1CCN(c2ccc(CCC(=O)NCCC(=O)NCCn3ccc(-c4cc(Cl)c(Cl)c5[nH]c6c(c45)CN(C(=O)CO)CC6)n3)s2)C(=O)C1]

Example 281: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(3-(5-(4-(4-(dimethylamino)but-2-enoyl)-2-oxopiperazin-1-yl)thiophen-2-yl)propanamido)propenamide (ZD178-63-2) Brown solid, 54% yield. ¹H NMR (400 MHz, CD₃OD) δ 7.76-7.61 (m, 1H), 7.29-7.09 (m, 1H), 7.00-6.69 (m, 2H), 6.62-6.39 (m, 3H), 4.64-4.15 (m, 8H), 4.05-3.59 (m, 10H), 3.45-3.34 (m, 2H), 3.13-2.76 (m, 10H), 2.50-2.25 (m, 4H). MS (ESI) [M+H]⁺=826.6.

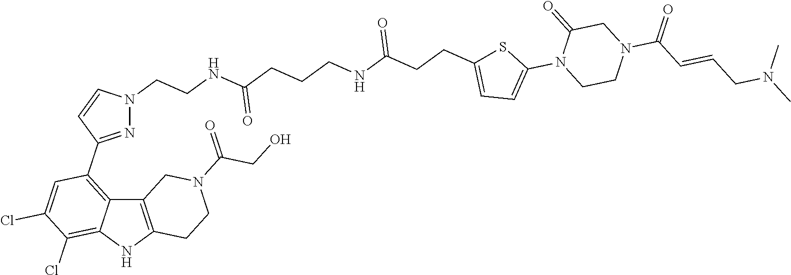 [SMILES: CN(C)C/C=C/C(=O)N1CCN(c2ccc(CCC(=O)NCCCC(=O)NCCn3ccc(-c4cc(Cl)c(Cl)c5[nH]c6c(c45)CN(C(=O)CO)CC6)n3)s2)C(=O)C1]

Example 282: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-4-(3-(5-(4-(4-(dimethylamino)but-2-enoyl)-2-oxopiperazin-1-yl)thiophen-2-yl)propanamido)butanamide (ZD178-63-3) Brown solid, 485 yield. ¹H NMR (400 MHz, CD₃OD) δ 7.76-7.62 (m, 1H), 7.28-7.15 (m, 1H), 7.00-6.67 (m, 2H), 6.64-6.35 (m, 3H), 4.68-4.15 (m, 8H), 4.07-3.51 (m, 10H), 3.19-2.78 (m, 12H), 2.55-2.36 (m, 2H), 2.19-2.03 (, 2H), 1.77-1.60 (n, 2H). MS (ESI) [M+H]⁺=840.7.

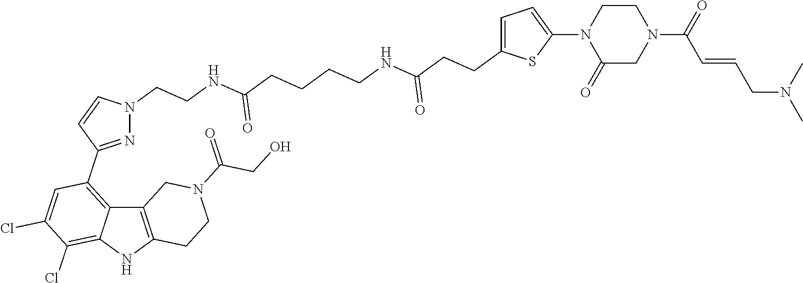 [SMILES: CN(C)C/C=C/C(=O)N1CCN(c2ccc(CCC(=O)NCCCCC(=O)NCCn3ccc(-c4cc(Cl)c(Cl)c5[nH]c6c(c45)CN(C(=O)CO)CC6)n3)s2)C(=O)C1]

Example 283: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-5-(3-(5-(4-(4-(dimethylamino)but-2-enoyl)-2-oxopiperazin-1-yl)thiophen-2-yl)propanamido)pentanamide (ZD178-63-4) Brown solid, 43% yield. ¹H NMR (400 MHz, CD₃OD) δ δ 7.71-7.57 (m, 1H), 7.29-7.12 (m, 1H), 7.01-6.66 (m, 2H), 6.63-6.42 (m, 3H), 4.63-4.20 (m, 8H), 4.07-3.62 (m, 10H), 3.15-2.83 (m, 12H), 2.55-2.33 (m, 2H), 2.15 (s, 2H), 1.59-1.25 (m, 4H). MS (ESI) [M+H]⁺=854.9.

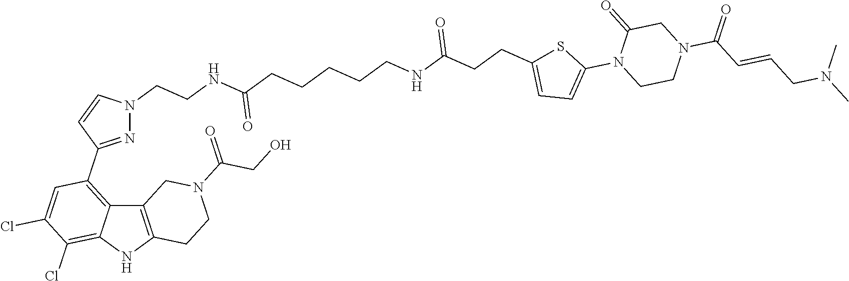 [SMILES: CN(C)C/C=C/C(=O)N1CCN(c2ccc(CCC(=O)NCCCCCC(=O)NCCn3ccc(-c4cc(Cl)c(Cl)c5[nH]c6c(c45)CN(C(=O)CO)CC6)n3)s2)C(=O)C1]

Example 284 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-6-(3-(5-(4-(4-(dimethylamino)but-2-enoyl)-2-oxopiperazin-1-yl)thiophen-2-yl)propanamido)hexanamide (ZD178-63-5) Brown solid, 55% yield. ¹H NMR (400 MHz, CD₃OD) δ 7.78-7.57 (m, 1H), 7.31-7.14 (m, 1H), 7.03-6.68 (m, 2H), 6.67-6.42 (m, 3H), 4.73-4.18 (m, 8H), 4.11-3.57 (m, 10H), 3.16-2.73 (m, 12H), 2.64-2.38 (m, 2H), 2.26-2.05 (m, 2H), 1.66-1.05 (m, 6H). MS (ESI) [M+H]⁺=868.5.

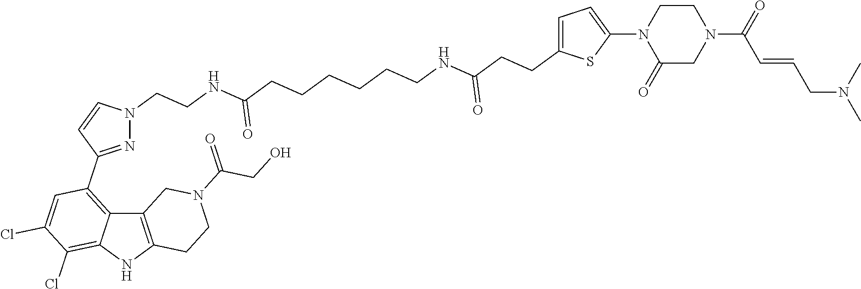 [SMILES: CN(C)C/C=C/C(=O)N1CCN(c2ccc(CCC(=O)NCCCCCCC(=O)NCCn3ccc(-c4cc(Cl)c(Cl)c5[nH]c6c(c45)CN(C(=O)CO)CC6)n3)s2)C(=O)C1]

Example 285 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-7-(3-(5-(4-(4-(dimethylamino)but-2-enoyl)-2-oxopiperazin-1-yl)thiophen-2-yl)propanamido)heptanamide (ZD178-63-6) Brown solid, 55% yield. ¹H NMR (400 MHz, CD₃OD) δ δ 7.78-7.51 (m, 1H), 7.30-7.14 (m, 1H), 7.07-6.68 (m, 2H), 6.68-6.33 (m, 3H), 4.76-4.18 (m, 8H), 4.12-3.61 (m, 10H), 3.15-2.78 (m, 12H), 2.61-2.41 (m, 2H), 2.34-2.01 (m, 2H), 1.70-0.88 (m, 8H). MS (ESI) [M+H]⁺=882.5.

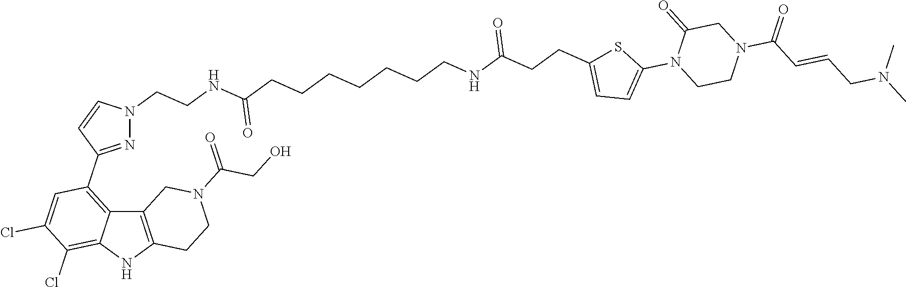 [SMILES: CN(C)C/C=C/C(=O)N1CCN(c2ccc(CCC(=O)NCCCCCCCC(=O)NCCn3ccc(-c4cc(Cl)c(Cl)c5[nH]c6c(c45)CN(C(=O)CO)CC6)n3)s2)C(=O)C1]

Example 286: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-8-(3-(5-(4-(4-(dimethylamino)but-2-enoyl)-2-oxopiperazin-1-yl)thiophen-2-yl)propanamido)octanamide (ZD178-63-7) Brown solid, 49% yield. ¹H NMR (400 MHz, CD₃OD) δ δ 7.78-7.58 (m, 1H), 7.32-7.12 (m, 1H), 7.04-6.68 (m, 2H), 6.66-6.36 (m, 3H), 4.69-4.11 (m, 8H), 4.08-3.61 (m, 10H), 3.16-2.75 (m, 12H), 2.57-2.37 (m, 2H), 2.27-2.03 (m, 2H), 1.65-1.06 (m, 10H). MS (ESI) [M+H]⁺=896.0.

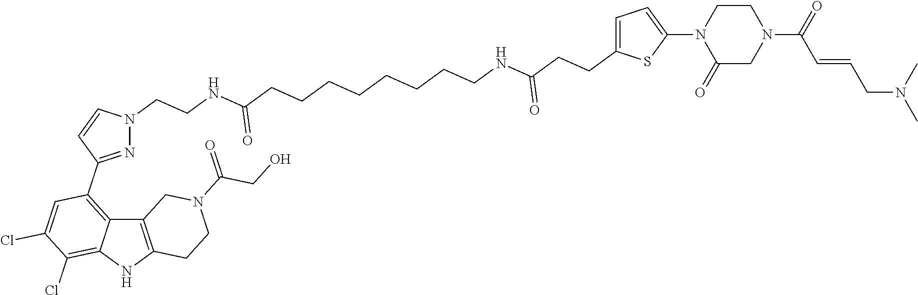 [SMILES: CN(C)C/C=C/C(=O)N1CCN(c2ccc(CCC(=O)NCCCCCCCCC(=O)NCCn3ccc(-c4cc(Cl)c(Cl)c5[nH]c6c(c45)CN(C(=O)CO)CC6)n3)s2)C(=O)C1]

Example 287: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-9-(3-(5-(4-(4-(dimethylamino)but-2-enoyl)-2-oxopiperazin-1-yl)thiophen-2-yl)propanamido)nonanamide (ZD178-63-8) Brown solid, 49% yield. ¹H NMR (400 MHz, CD₃OD) δ 7.77-7.55 (m, 1H), 7.31-7.13 (m, 1H), 7.04-6.70 (m, 2H), 6.71-6.41 (m, 3H), 4.68-4.19 (m, 8H), 4.11-3.61 (m, 10H), 3.18-2.79 (m, 12H), 2.60-2.38 (m, 2H), 2.30-1.92 (m, 2H), 1.74-0.81 (m, 12H). MS (ESI) [M+H]⁺=910.1.

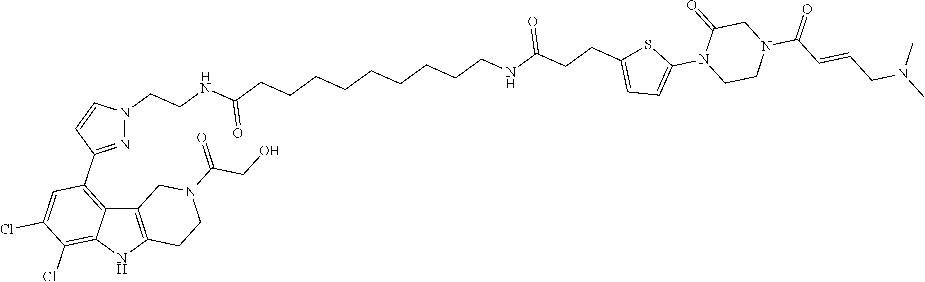 [SMILES: CN(C)C/C=C/C(=O)N1CCN(c2ccc(CCC(=O)NCCCCCCCCCC(=O)NCCn3ccc(-c4cc(Cl)c(Cl)c5[nH]c6c(c45)CN(C(=O)CO)CC6)n3)s2)C(=O)C1]

Example 288: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-10-(3-(5-(4-(4-(dimethylamino)but-2-enoyl)-2-oxopiperazin-1-yl)thiophen-2-yl)propanamido)decanamide (ZD178-63-9) Brown solid, 52% yield. ¹H NMR (400 MHz, CD₃OD) δ 7.80-7.51 (m, 1H), 7.41-7.07 (m, 1H), 7.09-6.70 (m, 2H), 6.68-6.34 (m, 3H), 4.71-4.17 (m, 8H), 4.19-3.54 (m, 10H), 3.27-2.77 (m, 12H), 2.62-2.41 (m, 2H), 2.30-1.99 (m, 2H), 1.66-0.97 (m, 14H). MS (ESI) [M+H]⁺=924.0.

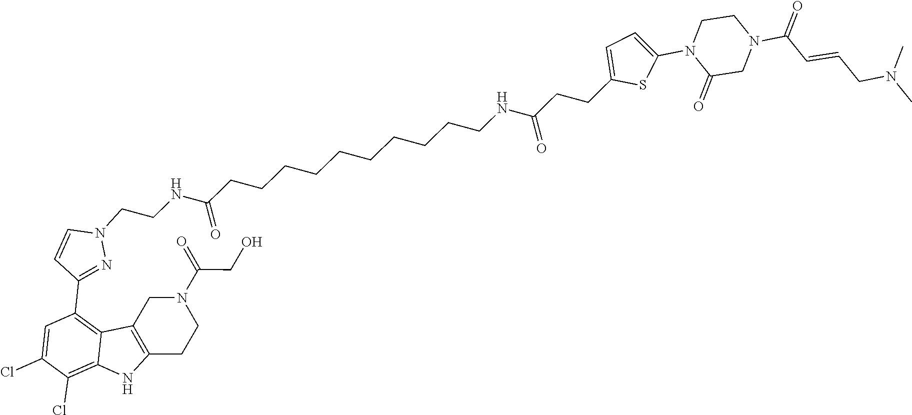 [SMILES: CN(C)C/C=C/C(=O)N1CCN(c2ccc(CCC(=O)NCCCCCCCCCCC(=O)NCCn3ccc(-c4cc(Cl)c(Cl)c5[nH]c6c(c45)CN(C(=O)CO)CC6)n3)s2)C(=O)C1]

Example 289: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-11-(3-(5-(4-(4-(dimethylamino)but-2-enoyl)-2-oxopiperazin-1-yl)thiophen-2-yl)propanamido)undecanamide (ZD178-63-10) Brown solid, 52% yield. ¹H NMR (400 MHz, CD₃OD) δ 7.79-7.55 (m, 1H), 7.21 (dt, J=12.6, 2.5 Hz, 1H), 7.09-6.70 (m, 2H), 6.68-6.34 (m, 3H), 4.68-4.18 (m, 8H), 4.10-3.63 (m, 10H), 3.22-2.80 (m, 12H), 2.61-2.36 (m, 2H), 2.28-2.05 (m, 2H), 1.70-0.89 (m, 16H). MS (ESI) [M+H]⁺=938.7.

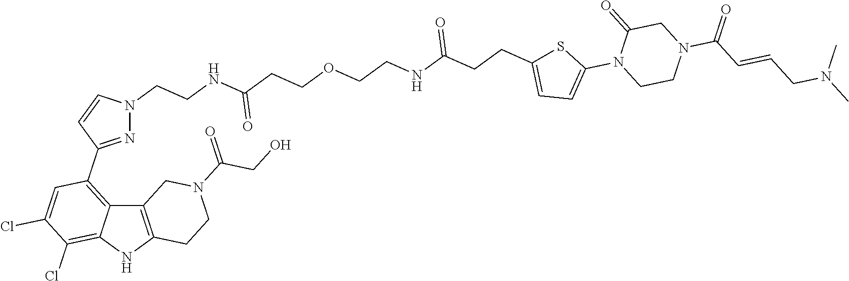 [SMILES: CN(C)C/C=C/C(=O)N1CCN(c2ccc(CCC(=O)NCCOCCC(=O)NCCn3ccc(-c4cc(Cl)c(Cl)c5[nH]c6c(c45)CN(C(=O)CO)CC6)n3)s2)C(=O)C1]

Example 290: (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(3-(5-(4-(4-(dimethylamino)but-2-enoyl)-2-oxopiperazin-1-yl)thiophen-2-yl)propanamido)ethoxy)propenamide (ZD178-63-11) Brown solid, 54% yield. ¹H NMR (400 MHz, CD₃OD) δ 7.73-7.59 (m, 1H), 7.30-7.13 (m, 1H), 7.04-6.66 (m, 2H), 6.63-6.34 (m, 3H), 4.64-4.14 (m, 8H), 4.06-3.54 (m, 12H), 3.48-3.38 (m, 2H), 3.28-3.21 (m, 2H), 3.09-2.83 (m, 10H), 2.63-2.33 (m, 4H). MS (ESI) [M+H]⁻=870.3.

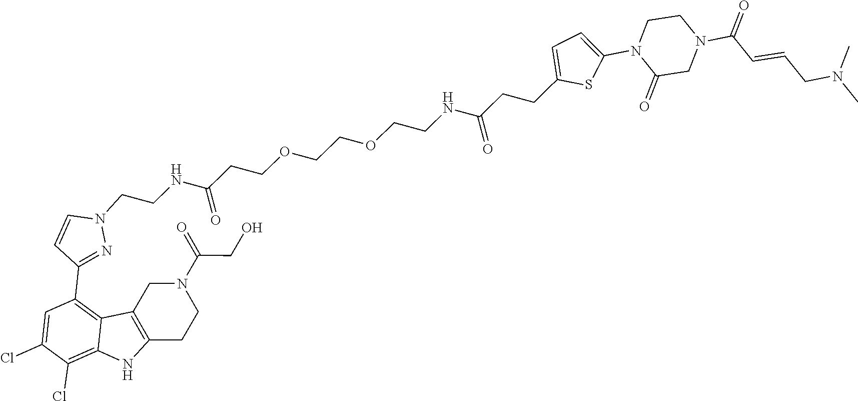 [SMILES: CN(C)C/C=C/C(=O)N1CCN(c2ccc(CCC(=O)NCCOCCOCCC(=O)NCCn3ccc(-c4cc(Cl)c(Cl)c5[nH]c6c(c45)CN(C(=O)CO)CC6)n3)s2)C(=O)C1]

Example 291 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(2-(3-(5-(4-(4-(dimethylamino)but-2-enoyl)-2-oxopiperazin-1-yl)thiophen-2-yl)propanamido)ethoxy)ethoxy)propenamide (ZD178-63-12) Brown solid, 40% yield. ¹H NMR (400 MHz, CD₃OD) δ 7.86-7.59 (m, 1H), 7.31-7.09 (m, 1H), 7.04-6.66 (m, 2H), 6.67-6.33 (m, 3H), 4.74-4.15 (m, 8H), 4.06-3.62 (m, 12H), 3.59-3.36 (m, 8H), 3.06-2.81 (m, 10H), 2.66-2.31 (m, 4H). MS (ESI) [M+H]⁺=914.6.

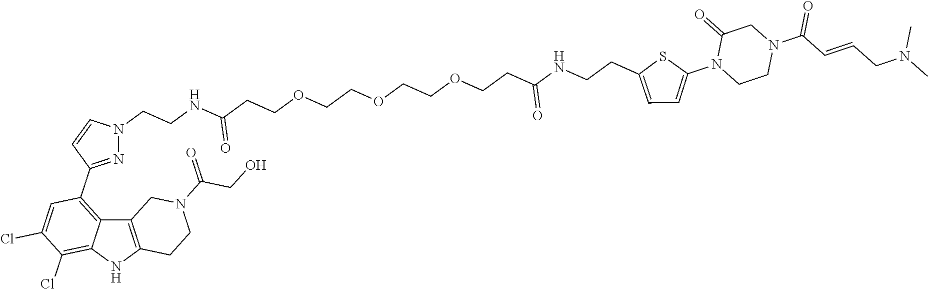 [SMILES: CN(C)C/C=C/C(=O)N1CCN(c2ccc(CCNC(=O)CCOCCOCCOCCC(=O)NCCn3ccc(-c4cc(Cl)c(Cl)c5[nH]c6c(c45)CN(C(=O)CO)CC6)n3)s2)C(=O)C1]

Example 292 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(2-(3-((2-(5-(4-(4-(dimethylamino)but-2-enoyl)-2-oxopiperazin-1-yl)thiophen-2-yl)ethyl)amino)-3-oxopropoxy)ethoxy)ethoxy)propenamide (ZD178-63-13) Brown solid, 45% yield. ¹H NMR (400 MHz, CD₃OD) δ 7.87-7.50 (m, 1H), 7.35-7.12 (m, 1H), 7.06-6.69 (m, 2H), 6.68-6.41 (m, 3H), 4.70-4.16 (m, 8H), 4.07-3.61 (m, 12H), 3.58-3.36 (m, 12H), 3.06-2.82 (m, 10H), 2.54-2.37 (m, 4H). MS (ESI) [M+H]⁺=958.7.

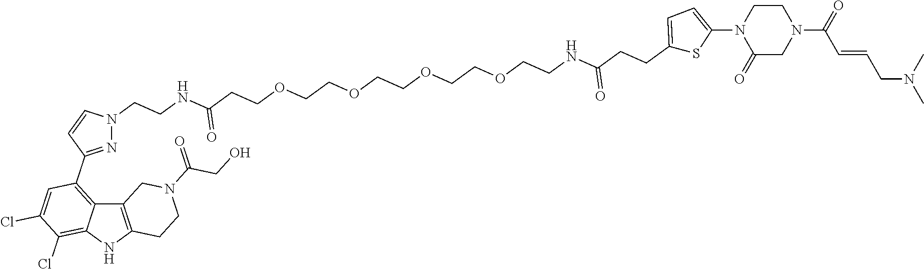 [SMILES: CN(C)C/C=C/C(=O)N1CCN(c2ccc(CCC(=O)NCCOCCOCCOCCOCCC(=O)NCCn3ccc(-c4cc(Cl)c(Cl)c5[nH]c6c(c45)CN(C(=O)CO)CC6)n3)s2)C(=O)C1]

Example 293 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-1-(3-(5-(4-(4-(dimethylamino)but-2-enoyl)-2-oxopiperazin-1-yl)thiophen-2-yl)propanamido)-3,6,9,12-tetraoxapentadecan-15-amide (ZD178-63-14) Brown solid, 46% yield. ¹H NMR (400 MHz, CD₃OD) δ 7.82-7.65 (m, 1H), 7.36-7.15 (m, 1H), 7.05-6.69 (m, 2H), 6.65-6.39 (m, 3H), 4.67-4.19 (m, 8H), 4.10-3.62 (m, 12H), 3.60-3.40 (m, 14H), 3.18-2.85 (m, 10H), 2.63-2.33 (m, 4H). MS (ESI) [M+H]⁺=1002.5.

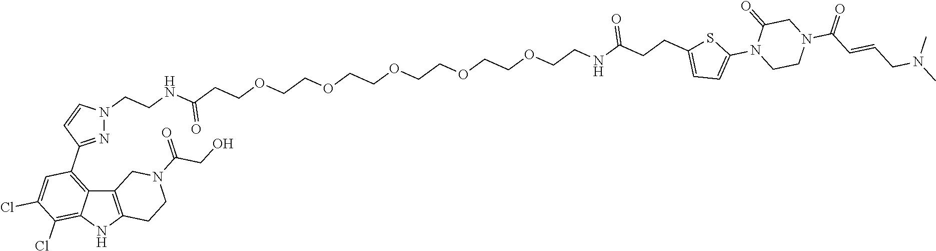 [SMILES: CN(C)C/C=C/C(=O)N1CCN(c2ccc(CCC(=O)NCCOCCOCCOCCOCCOCCC(=O)NCCn3ccc(-c4cc(Cl)c(Cl)c5[nH]c6c(c45)CN(C(=O)CO)CC6)n3)s2)C(=O)C1]

Example 294 (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-1-(3-(5-(4-(4-(dimethylamino)but-2-enoyl)-2-oxopiperazin-1-yl)thiophen-2-yl)propanamido)-3,6,9,12,15-pentaoxaoctadecan-18-amide (ZD178-63-15) Brown solid, 54% yield. ¹H NMR (400 MHz, CD₃OD) δ 7.78-7.64 (m, 1H), 7.30-7.15 (m, 1H), 7.05-6.67 (m, 2H), 6.68-6.36 (m, 3H), 4.70-4.10 (m, 8H), 4.09-3.62 (m, 12H), 3.60-3.41 (m, 18H), 3.12-2.82 (m, 10H), 2.63-2.32 (m, 4H). MS (ESI) [M+H]⁺=1046.4.

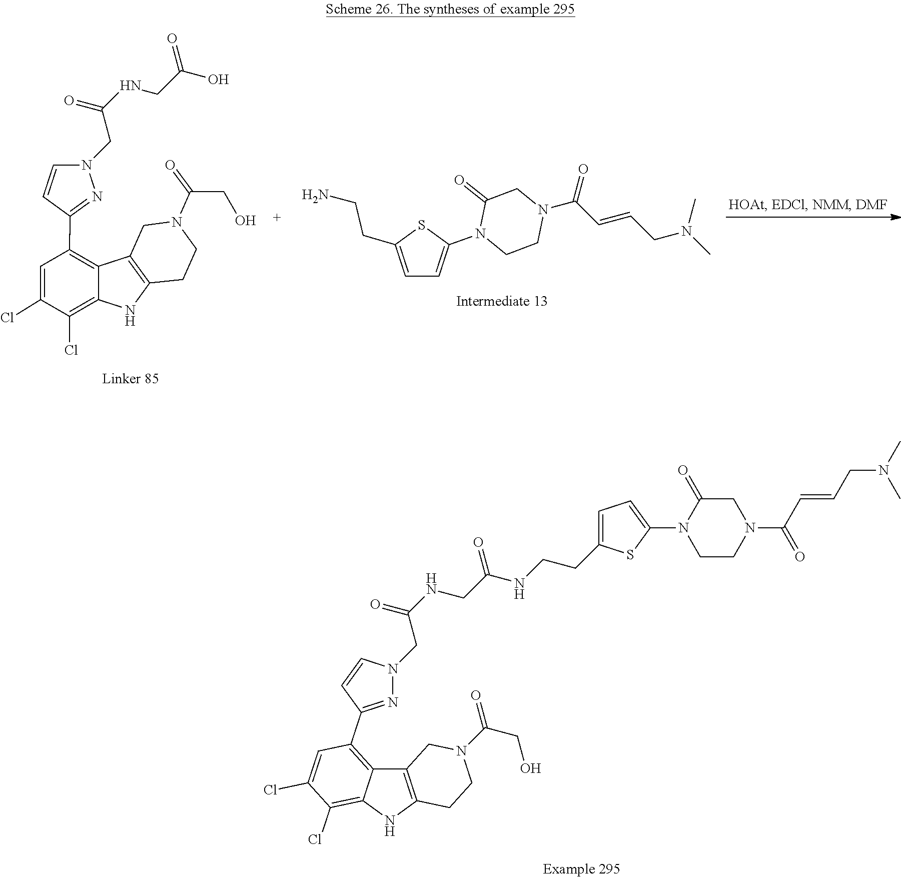 [SMILES: O=C(O)CNC(=O)Cn1ccc(-c2cc(Cl)c(Cl)c3[nH]c4c(c23)CN(C(=O)CO)CC4)n1 ; CN(C)C/C=C/C(=O)N1CCN(c2ccc(CCN)s2)C(=O)C1 ; CN(C)C/C=C/C(=O)N1CCN(c2ccc(CCNC(=O)CNC(=O)Cn3ccc(-c4cc(Cl)c(Cl)c5[nH]c6c(c45)CN(C(=O)CO)CC6)n3)s2)C(=O)C1]

Example 295: (E)-2-(3-(6,7-dichloro-2-(2-hydroxyacetyl)-2,3,4,5-tetrahydro-1H-pyrido[4,3-b]indol-9-yl)-1H-pyrazol-1-yl)-N-(2-((2-(5-(4-(4-(dimethylamino)but-2-enoyl)-2-oxopiperazin-1-yl)thiophen-2-yl)ethyl)amino)-2-oxoethyl)acetamide (ZD178-69-1) To a solution of Linker 85 (0.015 mmol, 1.0 eq) and Intermediate 13 (0.018 mmol, 1.2 eq) in dimethylformamide (1.0 mL) was added HOAt (0.03 mmol, 2.0 eq), NMM (0.15 mmol, 10.0 eq), and EDCI (0.03 mmol, 2.0 eq). The resulting reaction solution was stirred at rt for 12 h. Then the resulting residue was purified by reverse-phase chromatography to yield the desired product. Brown solid, 46% yield. ¹H NM/R (400 MHz, CD₃OD) δ 7.88-7.76 (m, 1H), 7.30-7.18 (m, 1H), 7.04-6.68 (m, 2H), 6.65-6.44 (m, 3H), 5.12-4.98 (m, 2H), 4.72-4.15 (m, 6H), 4.06-3.65 (m, 10H), 3.25-3.12 (m, 2H), 3.01-2.77 (m, 10H). MS (ESI) [M+H]⁺=798.0.

Example 296-309 were synthesized following the same procedure for preparing example 295 from related linkers and intermediate 13.

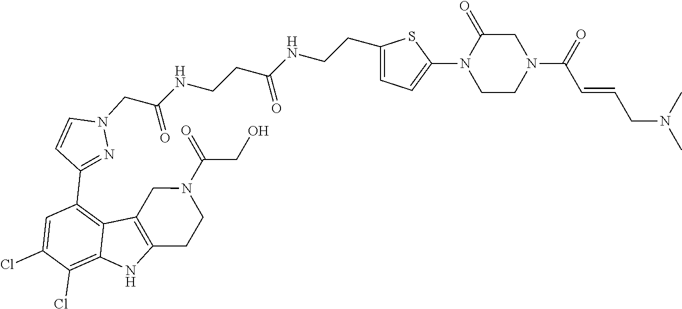 [SMILES: CN(C)C/C=C/C(=O)N1CCN(c2ccc(CCNC(=O)CCNC(=O)Cn3ccc(-c4cc(Cl)c(Cl)c5[nH]c6c(c45)CN(C(=O)CO)CC6)n3)s2)C(=O)C1]

Example 296 (E)-3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)propenamide (ZD178-69-2) Brown solid, 49% yield. ¹H NMR (400 MHz, CD₃OD) δ 7.88-7.60 (m, 1H), 7.37-7.08 (m, 1H), 7.04-6.68 (m, 2H), 6.68-6.41 (m, 3H), 5.04-4.90 (m, 2H), 4.73-4.08 (m, 6H), 4.04-3.65 (m, 8H), 3.56-3.35 (m, 4H), 3.03-2.71 (m, 10H), 2.51-2.30 (m, 2H). MS (ESI) [M+H]⁺=812.4.

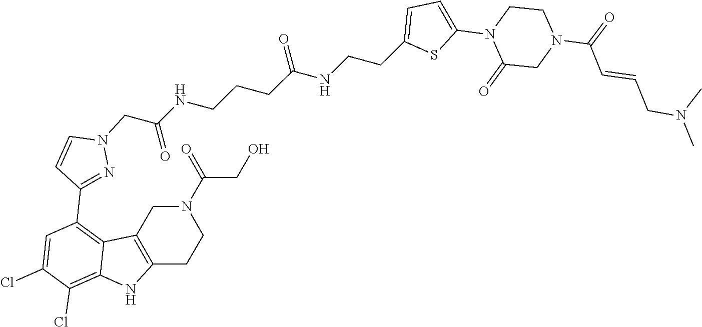 [SMILES: CN(C)C/C=C/C(=O)N1CCN(c2ccc(CCNC(=O)CCCNC(=O)Cn3ccc(-c4cc(Cl)c(Cl)c5[nH]c6c(c45)CN(C(=O)CO)CC6)n3)s2)C(=O)C1]

Example 297 (E)-4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)butanamide (ZD178-69-3) Brown solid, 49% yield. ¹H NMR (400 MHz, CD₃OD) δ 7.83-7.74 (m, 1H), 7.29-7.17 (m, 1H), 7.02-6.68 (m, 2H), 6.67-6.50 (m, 3H), 5.03-4.92 (m, 2H), 4.71-4.14 (m, 6H), 4.05-3.66 (m, 8H), 3.46-3.35 (m, 2H), 3.27-3.12 (m, 4H), 3.03-2.80 (m, 8H), 2.34-2.11 (m, 2H), 1.80 (s, 2H). MS (ESI) [M+H]⁺=826.4.

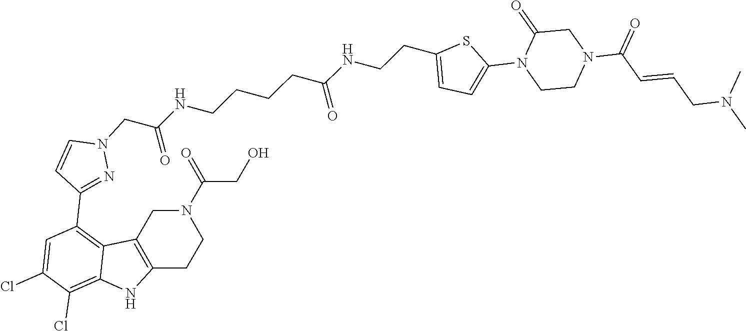 [SMILES: CN(C)C/C=C/C(=O)N1CCN(c2ccc(CCNC(=O)CCCCNC(=O)Cn3ccc(-c4cc(Cl)c(Cl)c5[nH]c6c(c45)CN(C(=O)CO)CC6)n3)s2)C(=O)C1]

Example 298 (E)-5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)pentanamide (ZD178-69-4) Brown solid, 51% yield. ¹H NMR (400 MHz, CD₃OD) δ 7.86-7.65 (m, 1H), 7.36-7.12 (m, 1H), 6.98-6.68 (m, 2H), 6.68-6.49 (m, 3H), 5.06-4.92 (m, 2H), 4.74-4.14 (m, 6H), 4.07-3.66 (m, 8H), 3.46-3.36 (m, 2H), 3.28-3.16 (m, 4H), 3.03-2.79 (m, 8H), 2.31-2.02 (m, 2H), 1.73-1.42 (m, 4H). MS (ESI) [M+H]⁺=840.4.

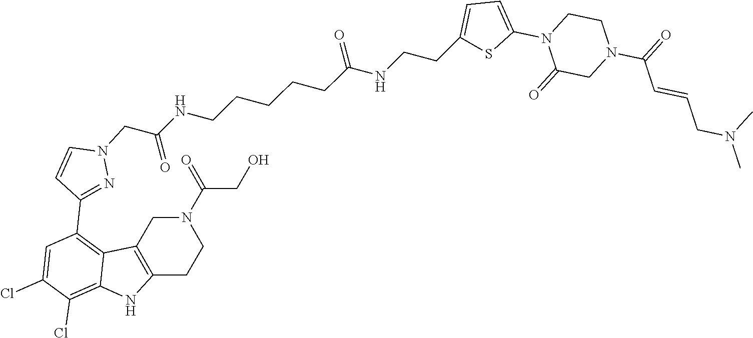 [SMILES: CN(C)C/C=C/C(=O)N1CCN(c2ccc(CCNC(=O)CCCCCNC(=O)Cn3ccc(-c4cc(Cl)c(Cl)c5[nH]c6c(c45)CN(C(=O)CO)CC6)n3)s2)C(=O)C1]

Example 299 (E)-6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)hexanamide (ZD178-69-5) Brown solid, 51% yield. ¹H NMR (400 MHz, CD₃OD) δ 7.86-7.69 (m, 1H), 7.29-7.14 (m, 1H), 7.03-6.69 (m, 2H), 6.69-6.53 (m, 3H), 5.02-4.90 (m, 2H), 4.77-4.11 (m, 6H), 4.08-3.66 (m, 8H), 3.56-3.35 (m, 2H), 3.26-3.09 (m, 4H), 3.03-2.79 (m, 8H), 2.27-2.04 (m, 2H), 1.71-1.12 (m, 6H). MS (ESI) [M+H]⁺=854.4.

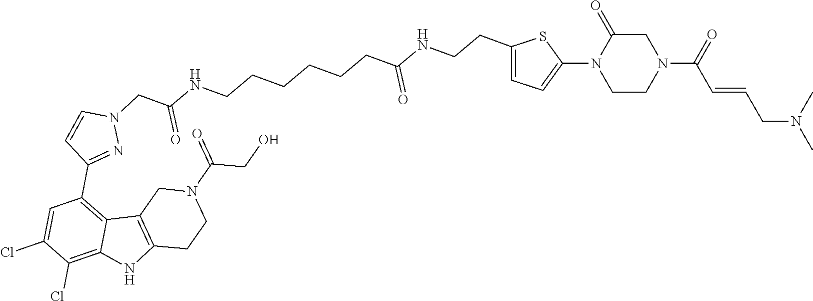 [SMILES: CN(C)C/C=C/C(=O)N1CCN(c2ccc(CCNC(=O)CCCCCCNC(=O)Cn3ccc(-c4cc(Cl)c(Cl)c5[nH]c6c(c45)CN(C(=O)CO)CC6)n3)s2)C(=O)C1]

Example 300 (E)-7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)heptanamide (ZD178-69-6) Brown solid, 53% yield. ¹H NMR (400 MHz, CD₃OD) δ 7.97-7.64 (m, 1H), 7.53-7.18 (m, 1H), 7.02-6.70 (m, 2H), 6.70-6.50 (m, 3H), 5.09-4.92 (m, 2H), 4.73-4.14 (m, 6H), 4.10-3.66 (m, 8H), 3.49-3.36 (m, 2H), 3.27-3.11 (m, 4H), 3.04-2.82 (m, 8H), 2.28-2.04 (m, 2H), 1.72-1.09 (m, 8H). MS (ESI) [M+H]⁺=868.4.

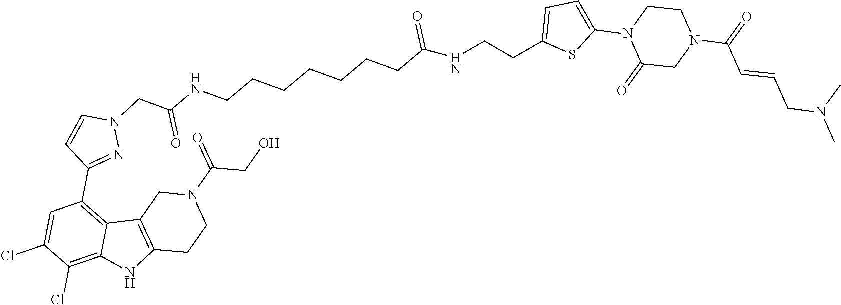 [SMILES: CN(C)C/C=C/C(=O)N1CCN(c2ccc(CCNC(=O)CCCCCCCNC(=O)Cn3ccc(-c4cc(Cl)c(Cl)c5[nH]c6c(c45)CN(C(=O)CO)CC6)n3)s2)C(=O)C1]

Example 301: (E)-8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)octanamide (ZD178-69-7) Brown solid, 53% yield. ¹H NMR (400 MHz, CD₃OD) δ 7.97-7.58 (m, 1H), 7.47-7.12 (m, 1H), 7.05-6.70 (m, 2H), 6.74-6.42 (m, 3H), 5.01-4.91 (m, 2H), 4.71-4.16 (m, 6H), 4.05-3.64 (m, 8H), 3.48-3.36 (m, 2H), 3.27-3.17 (m, 4H), 2.99-2.72 (m, 8H), 2.29-1.99 (m, 2H), 1.71-1.15 (m, 10H). MS (ESI) [M+H]⁺=882.5.

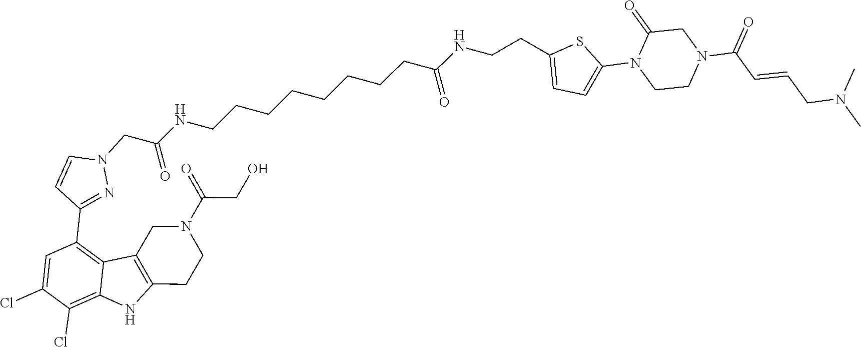 [SMILES: CN(C)C/C=C/C(=O)N1CCN(c2ccc(CCNC(=O)CCCCCCCCNC(=O)Cn3ccc(-c4cc(Cl)c(Cl)c5[nH]c6c(c45)CN(C(=O)CO)CC6)n3)s2)C(=O)C1]

Example 302: (E)-9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)nonanamide (ZD178-69-8) Brown solid, 49% yield. ¹H NMR (400 MHz, CD₃OD) δ 7.97-7.70 (m, 1H), 7.29-7.21 (m, 1H), 7.03-6.70 (m, 2H), 6.70-6.51 (m, 3H), 5.04-4.90 (m, 2H), 4.81-4.15 (m, 6H), 4.10-3.67 (m, 8H), 3.50-3.37 (m, 2H), 3.27-3.12 (m, 4H), 3.08-2.64 (m, 8H), 2.51-1.97 (m, 2H), 1.78-0.65 (m, 12H). MS (ESI) [M+H]⁺=896.3.

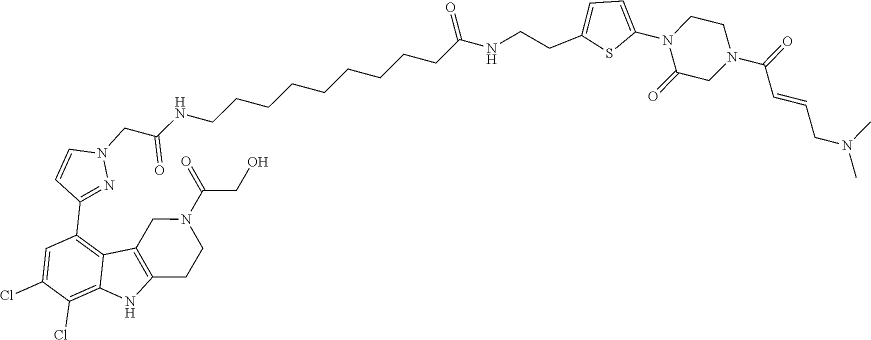 [SMILES: CN(C)C/C=C/C(=O)N1CCN(c2ccc(CCNC(=O)CCCCCCCCCNC(=O)Cn3ccc(-c4cc(Cl)c(Cl)c5[nH]c6c(c45)CN(C(=O)CO)CC6)n3)s2)C(=O)C1]

Example 303: (E)-10-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)decanamide (ZD178-69-9) Brown solid, 49% yield. ¹H NMR (400 MHz, CD₃OD) δ 7.91-7.67 (m, 1H), 7.35-7.14 (m, 1H), 7.02-6.70 (m, 2H), 6.71-6.52 (m, 3H), 5.09-4.90 (m, 2H), 4.78-4.16 (m, 6H), 4.08-3.61 (m, 8H), 3.51-3.37 (m, 2H), 3.28-3.17 (m, 4H), 3.07-2.66 (m, 8H), 2.22-2.02 (m, 2H), 1.67-1.10 (m, 14H). MS (ESI) [M+H]⁺=910.4.

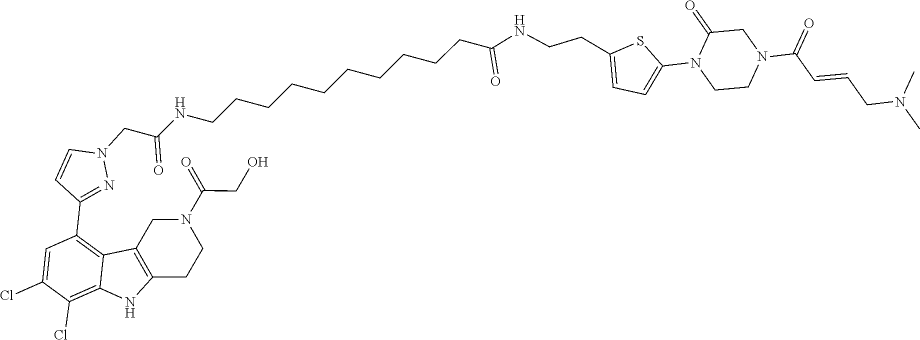 [SMILES: CN(C)C/C=C/C(=O)N1CCN(c2ccc(CCNC(=O)CCCCCCCCCCNC(=O)Cn3ccc(-c4cc(Cl)c(Cl)c5[nH]c6c(c45)CN(C(=O)CO)CC6)n3)s2)C(=O)C1]

Example 304 (E)-11-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)undecanamide (ZD178-69-9) Brown solid, 52% yield. H NMR (400 MHz, CD₃OD) δ 7.87-7.58 (m, 1H), 7.49-7.20 (m, 1H), 7.03-6.70 (m, 2H), 6.72-6.43 (m, 3H), 5.01-4.91 (m, 2H), 4.72-4.15 (m, 6H), 4.08-3.65 (m, 8H), 3.45-3.37 (m, 2H), 3.27-3.15 (m, 4H), 3.03-2.79 (m, 8H), 2.34-2.05 (m, 2H), 1.75-1.02 (m, 16H).MS (ESI) [M+H]⁺=924.4.

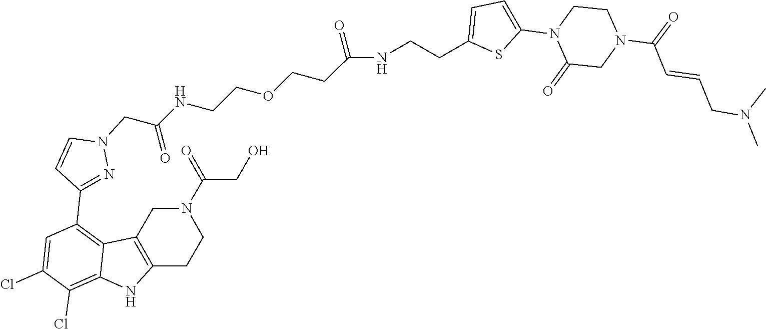 [SMILES: CN(C)C/C=C/C(=O)N1CCN(c2ccc(CCNC(=O)CCOCCNC(=O)Cn3ccc(-c4cc(Cl)c(Cl)c5[nH]c6c(c45)CN(C(=O)CO)CC6)n3)s2)C(=O)C1]

Example 305 (E)-3-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)-N-(2-(5-(4-(4-(dimethylamino)but-2-enoyl)-2-oxopiperazin-1-yl)thiophen-2-yl)ethyl)propenamide (ZD178-69-11) Brown solid, 47% yield. ¹H NMR (400 MHz, CD₃OD) δ 8.04-7.66 (m, 1H), 7.41-7.16 (m, 1H), 7.07-6.68 (m, 2H), 6.72-6.43 (m, 3H), 5.11-4.90 (m, 2H), 4.73-4.12 (m, 6H), 4.04-3.60 (m, 10H), 3.57-3.35 (m, 6H), 2.97-2.83 (m, 10H), 2.50-2.31 (m, 2H). MS (ESI) [M+H]⁺=856.9.

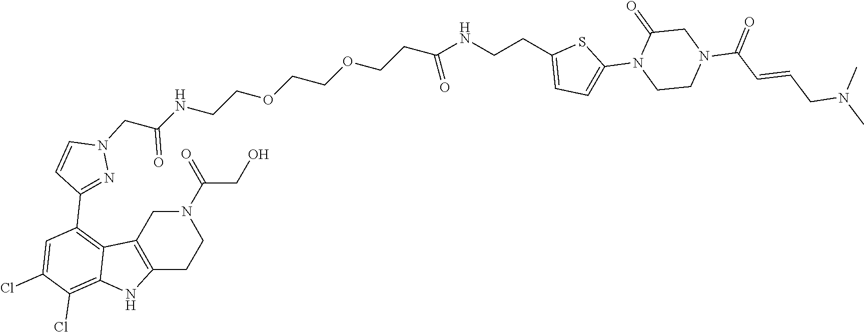 [SMILES: CN(C)C/C=C/C(=O)N1CCN(c2ccc(CCNC(=O)CCOCCOCCNC(=O)Cn3ccc(-c4cc(Cl)c(Cl)c5[nH]c6c(c45)CN(C(=O)CO)CC6)n3)s2)C(=O)C1]

Example 306 (E)-3-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethoxy)-N-(2-(5-(4-(4-(dimethylamino)but-2-enoyl)-2-oxopiperazin-1-yl)thiophen-2-yl)ethyl)propenamide (ZD178-69-12) Brown solid, 50% yield. ¹H NMR (400 MHz, CD₃OD) δ 7.99-7.45 (m, 1H), 7.43-7.05 (m, 1H), 6.99-6.70 (m, 2H), 6.69-6.45 (m, 3H), 5.05-4.92 (m, 2H), 4.74-4.17 (m, 6H), 4.03-3.59 (m, 10H), 3.59-3.34 (m, 10H), 3.02-2.81 (m, 10H), 2.52-2.33 (m, 2H). MS (ESI) [M+H]⁺=900.4.

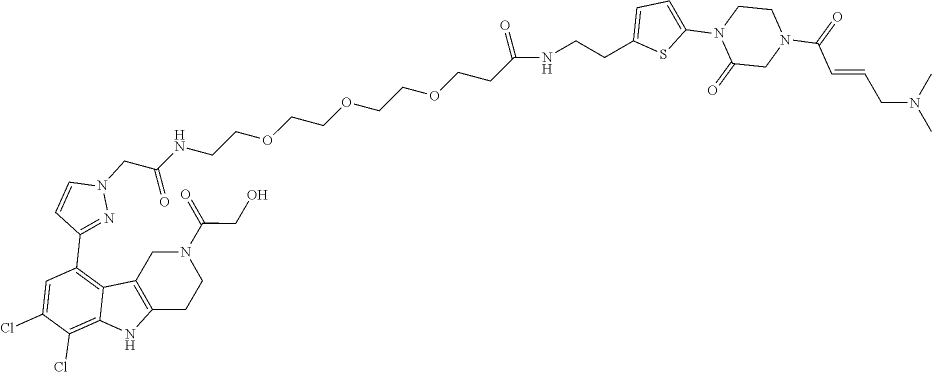 [SMILES: CN(C)C/C=C/C(=O)N1CCN(c2ccc(CCNC(=O)CCOCCOCCOCCNC(=O)Cn3ccc(-c4cc(Cl)c(Cl)c5[nH]c6c(c45)CN(C(=O)CO)CC6)n3)s2)C(=O)C1]

Example 307:(E)-3-(2-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethoxy)ethoxy)-N-(2-(5-(4-(4-(dimethylamino)but-2-enoyl)-2-oxopiperazin-1-yl)thiophen-2-yl)ethyl)propenamide (ZD178-69-13) Brown solid, 33% yield. ¹H NMR (400 MHz, CD₃OD) δ 7.95-7.56 (m, 1H), 7.45-7.14 (m, 1H), 7.01-6.70 (m, 2H), 6.70-6.52 (m, 3H), 5.07-4.91 (m, 2H), 4.71-4.12 (m, 6H), 4.09-3.62 (m, 8H), 3.60-3.35 (m, 16H), 3.05-2.76 (m, 10H), 2.53-2.26 (m, 2H). MS (ESI) [M+H]⁻=944.0.

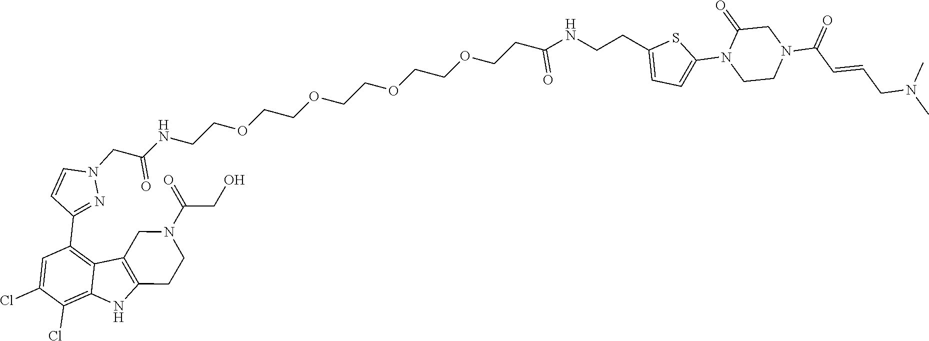 [SMILES: CN(C)C/C=C/C(=O)N1CCN(c2ccc(CCNC(=O)CCOCCOCCOCCOCCNC(=O)Cn3ccc(-c4cc(Cl)c(Cl)c5[nH]c6c(c45)CN(C(=O)CO)CC6)n3)s2)C(=O)C1]

Example 308 (E)-1-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)-3,6,9,12-tetraoxapentadecan-15-amide (ZD178-69-14) Brown solid, 56% yield. ¹H NMR (400 MHz, CD₃OD) δ 7.99-7.62 (m, 1H), 7.46-7.19 (m, 1H), 7.03-6.69 (m, 2H), 6.69-6.49 (m, 3H), 5.07-4.93 (m, 2H), 4.78-4.18 (m, 6H), 4.12-3.62 (m, 10H), 3.62-3.34 (m, 18H), 3.05-2.79 (m, 10H), 2.51-2.33 (m, 2H). MS (ESI) [M+H]⁺=988.1.

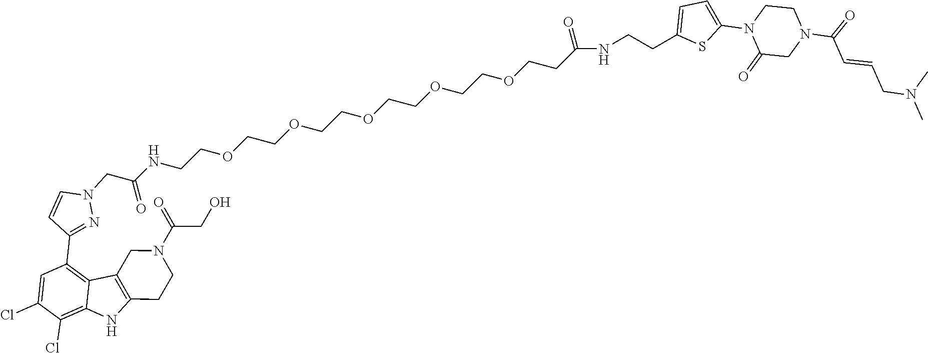 [SMILES: CN(C)C/C=C/C(=O)N1CCN(c2ccc(CCNC(=O)CCOCCOCCOCCOCCOCCNC(=O)Cn3ccc(-c4cc(Cl)c(Cl)c5[nH]c6c(c45)CN(C(=O)CO)CC6)n3)s2)C(=O)C1]

Example 309 (E)-1-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(2-(5-(4-(4-(dimethylamino)but-2-enoyl)-2-oxopiperazin-1-yl)thiophen-2-yl)ethyl)-3,6,9,12,15-pentaoxaoctadecan-18-amide (ZD178-69-15) Brown solid, 50% yield. ¹H NMR (400 MHz, CD₃OD) δ 7.87-7.72 (m, 1H), 7.32-7.21 (m, 1H), 7.01-6.69 (m, 2H), 6.69-6.54 (m, 3H), 5.04-4.94 (m, 2H), 4.71-4.18 (m, 6H), 4.07-3.63 (m, 10H), 3.61-3.36 (m, 22H), 2.98-2.81 (m, 10H), 2.52-2.37 (m, 2H). MS (ESI) [M+H]⁺=1032.4.

### Procedures for the Synthesis of CFTR Based Bivalent Compounds

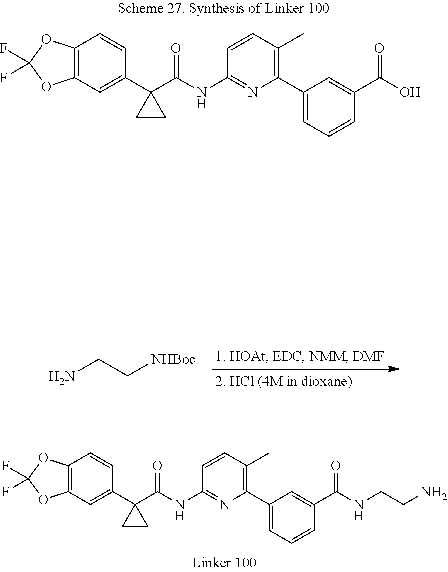 [SMILES: Cc1ccc(NC(=O)C2(c3ccc4c(c3)OC(F)(F)O4)CC2)nc1-c1cccc(C(=O)O)c1 ; CC(C)(C)OC(=O)NCCN ; Cc1ccc(NC(=O)C2(c3ccc4c(c3)OC(F)(F)O4)CC2)nc1-c1cccc(C(=O)NCCN)c1]

Linker 100: N-(2-aminoethyl)-3-(6-(1-(2,2-difluorobenzo[d][1,3]dioxol-5-yl)cyclopropane-1-carboxamido)-3-methylpyridin-2-yl)benzamide 3-(6-(1-(2,2-difluorobenzo[d][1,3]dioxol-5-yl)cyclopropane-1-carboxamido)-3-methylpyridin-2-yl)benzoic acid (100 mg, 0.22 mmol, 1.0 eq), tert-butyl (2-aminoethyl)carbamate (38 mg, 0.24 mmol, 1.1 eq), EDC HCl (63 mg, 0.33 mmol, 1.5 eq), HOAt (45 mg, 0.33 mmol, 1.5 eq), NMM (67 mg, 0.66 mmol, 3.0 eq) were stirred in DMF (1.5 mL) at room temperature for 2 h. Then the mixture was purified via reverse-ISCO to get related intermediate. Then the intermediate was dissolved in MeOH (1 mL) followed by HCl in dioxane (4 M, 1 mL, 4 mmol), the mixture was stirred at room temperature for 2 h. After removed all the volatiles, the titled compound was obtained as a white solid (50 mg, 45% yield over two steps). ¹H NMR (400 MHz, CD₃OD) δ 8.13 (d, J=8.8 Hz, 1H), 8.07 (d, J=7.6 Hz, 1H), 8.02-7.98 (m, 2H), 7.70-7.63 (m, 2H), 7.37-7.30 (m, 2H), 7.23 (d, J=10.8 Hz, 1H), 3.80-3.77 (m, 2H), 3.33-3.29 (m, 2H), 2.36 (s, 3H), 1.77-1.73 (m, 2H), 1.35-1.31 (m, 2H).

Linkers 101-113 are synthesized as the same procedure for preparing linker 100.

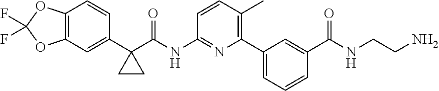 [SMILES: Cc1ccc(NC(=O)C2(c3ccc4c(c3)OC(F)(F)O4)CC2)nc1-c1cccc(C(=O)NCCN)c1]

Linker 101: N-(3-aminopropyl)-3-(6-(1-(2,2-difluorobenzo[d][1,3]dioxol-5-yl)cyclopropane-1-carboxamido)-3-methylpyridin-2-yl)benzamide White solid, 61% yield. ¹H NMR (400 MHz, CD₃OD) δ 8.12 (d, J=9.0 Hz, 1H), 8.08-8.00 (m, 3H), 7.75-7.66 (m, 2H), 7.41 (s, 1H), 7.35 (d, J=8.3 Hz, 1H), 7.25 (d, J=8.3 Hz, 1H), 3.53 (t, J=5.9 Hz, 2H), 3.02 (t, J=7.3 Hz, 2H), 2.35 (s, 3H), 1.99 (p, J=6.9 Hz, 2H), 1.77-1.73 (m, 2H), 1.37-1.33 (m, 2H).

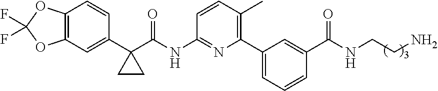

Linker 102: N-(4-aminobutyl)-3-(6-(1-(2,2-difluorobenzo[d][1,3]dioxol-5-yl)cyclopropane-1-carboxamido)-3-methylpyridin-2-yl)benzamide White solid, 60% yield. ¹H NMR (400 MHz, CD₃OD) δ 8.09 (d, J=8.4 Hz, 1H), 7.89 (d, J=5.4 Hz, 1H), 7.84 (s, 1H), 7.80 (d, J=8.6 Hz, 1H), 7.60-7.55 (m, 2H), 7.40 (s, 1H), 7.34 (d, J=8.3 Hz, 1H), 7.23 (d, J=8.3 Hz, 1H), δ 3.37-3.28 (m, 2H), 2.85 (t, J=7.7 Hz, 2H).2.26 (s, 3H), 1.77-1.66 (m, 6H), 1.30-1.20 (m, 2H).

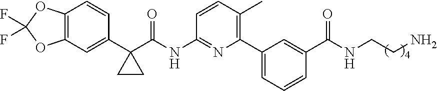

Linker 103: N-(5-aminopentyl)-3-(6-(1-(2,2-difluorobenzo[d][1,3]dioxol-5-yl)cyclopropane-1-carboxamido)-3-methylpyridin-2-yl)benzamide White solid, 65% yield. ¹H NMR (400 MHz, CD₃OD) δ 8.19-7.84 (m, 4H), 7.67-7.53 (m, 2H), 7.30 (s, 1H), 7.24 (d, J=7.9 Hz, 1H), 7.15 (d, J=8.9 Hz, 1H), 3.33 (t, J=7.8 Hz, 2H), 2.84 (t, J=7.6 Hz, 2H), 2.31-2.20 (m, 3H), 1.69-1.54 (m, 6H), 1.45-1.35 (m, 2H), 1.30-1.21 (m, 2H).

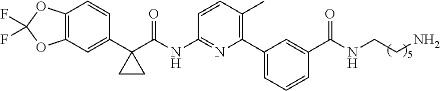

Linker 104: N-(6-aminohexyl)-3-(6-(1-(2,2-difluorobenzo[d][1,3]dioxol-5-yl)cyclopropane-1-carboxamido)-3-methylpyridin-2-yl)benzamide White solid, 62% yield. ¹H NMR (400 MHz, CD₃OD) δ 8.39 (d, J=8.0 Hz, 1H), 8.16-8.06 (m, 3H), 7.80-7.70 (m, 2H), 7.35-7.30 (m, 2H), 7.20-7.18 (m, 1H), 3.38-3.34 (m, 2H), 2.93-2.89 (m, 2H), 2.38 (s, 3H), 1.75-1.63 (m, 6H), 1.42-1.34 (m, 6H).

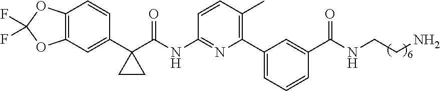

Linker 105: N-(7-aminoheptyl)-3-(6-(1-(2,2-difluorobenzo[d][1,3]dioxol-5-yl)cyclopropane-1-carboxamido)-3-methylpyridin-2-yl)benzamide White solid, 64% yield. ¹H NMR (400 MHz, CD₃OD) δ 8.38 (d, J=8.0 Hz, 1H), 8.12-8.04 (m, 3H), 7.81-7.70 (m, 2H), 7.35-7.30 (m, 2H), 7.19 (d, J=7.2 Hz, 1H), 3.37-3.34 (m, 2H), 2.91-2.87 (m, 2H), 2.38 (s, 3H), 1.74-1.61 (m, 6H), 1.38-1.34 (m, 8H).

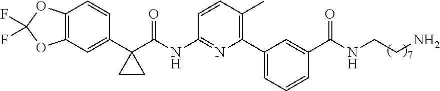

Linker 106: N-(8-aminooctyl)-3-(6-(1-(2,2-difluorobenzo[d][1,3]dioxol-5-yl)cyclopropane-1-carboxamido)-3-methylpyridin-2-yl)benzamide White solid, 60% yield. ¹H NMR (400 MHz, CD₃OD) δ 8.38 (d, J=8.0 Hz, 1H), 8.14-8.04 (m, 3H), 7.81-7.70 (m, 2H), 7.35-7.30 (m, 2H), 7.19-7.18 (m, 1H), 3.36-3.34 (m, 2H), 2.89 (t, J=7.6 Hz, 2H), 2.38 (s, 3H), 1.80-1.53 (m, 6H), 1.37-1.33 (m, 10H).

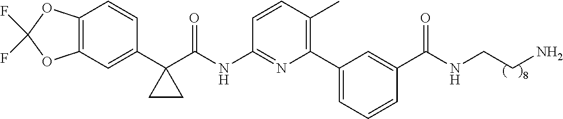

Linker 107: N-(9-aminononyl)-3-(6-(1-(2,2-difluorobenzo[d][1,3]dioxol-5-yl)cyclopropane-1-carboxamido)-3-methylpyridin-2-yl)benzamide White solid, 59% yield. ¹H NMR (400 MHz, CD₃OD) δ 8.38 (d, J=8.0 Hz, 1H), 8.14-8.04 (m, 3H), 7.81-7.70 (m, 2H), 7.35-7.30 (m, 2H), 7.20-7.18 (m, 1H), 3.35-3.37 (m, 2H), 2.89 (t, J=8.0 Hz, 2H), 2.38 (s, 3H), 1.75 (s, 2H), 1.66-1.58 (m, 4H), 1.34-1.33 (m, 12H).

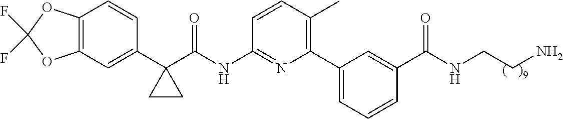

Linker 108: N-(10-aminodecyl)-3-(6-(1-(2,2-difluorobenzo[d][1,3]dioxol-5-yl)cyclopropane-1-carboxamido)-3-methylpyridin-2-yl)benzamide White solid, 64% yield. ¹H NMR (400 MHz, CD₃OD) δ 8.39 (s, 1H), 8.18-8.05 (m, 3H), 7.82-7.69 (m, 2H), 7.33-7.30 (m, 2H), 7.18 (s, 1H), 3.34-7.32 (m, 2H), 2.89 (s, 2H), 2.39 (s, 3H), 1.75-1.59 (m, 6H), 1.33-1.30 (m, 14H).

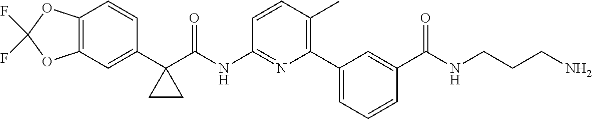 [SMILES: Cc1ccc(NC(=O)C2(c3ccc4c(c3)OC(F)(F)O4)CC2)nc1-c1cccc(C(=O)NCCCN)c1]

Linker 109: N-(2-(2-aminoethoxy)ethyl)-3-(6-(1-(2,2-difluorobenzo[d][1,3]dioxol-5-yl)cyclopropane-1-carboxamido)-3-methylpyridin-2-yl)benzamide White solid, 62% yield. ¹H NMR (400 MHz, CD₃OD) δ 8.39 (d, J=8.4 Hz, 1H), 8.19-8.11 (m, 3H), 7.83-7.81 (m, 1H), 7.71 (t, J=7.6 Hz, 1H), 7.34-7.30 (m, 2H), 7.20-7.18 (m, 1H), 3.72-3.68 (m, 4H), 3.61-3.55 (m, 2H), 3.14-3.08 (m, 2H), 2.39 (s, 3H), 1.77-1.73 (m, 2H), 1.36-1.31 (m, 2H).

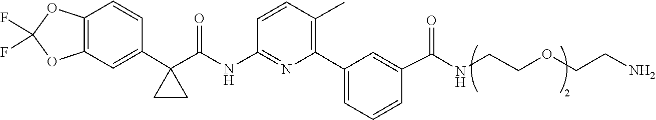

Linker 110: N-(2-(2-(2-aminoethoxy)ethoxy)ethyl)-3-(6-(1-(2,2-difluorobenzo[d][1,3]dioxol-5-yl)cyclopropane-1-carboxamido)-3-methylpyridin-2-yl)benzamide White solid, 60% yield. ¹H NMR (400 MHz, CD₃OD) δ 8.08 (d, J=8.6 Hz, 1H), 7.84-7.77 (m, 3H), 7.52-7.50 (m, 1H), 7.41 (t, J=8.0 Hz, 1H), 7.04-7.00 (m, 2H), 6.91-6.88 (m, 1H), 3.39-3.36 (m, 8H), 3.28-3.27 (m, 2H), 2.79-2.75 (m, 2H), 2.09 (s, 3H), 1.48-1.42 (m, 2H), 1.08-1.02 (m, 2H).

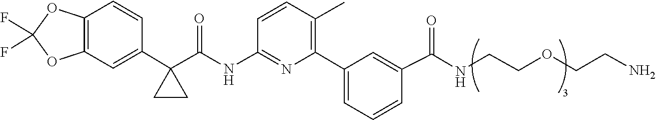

Linker 111: N-(2-(2-(2-(2-aminoethoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide White solid, 61% yield. ¹H NMR (400 MHz, CD₃OD) δ 7.70 (d, J=8.0 Hz, 1H), 7.59-7.50 (m, 3H), 7.22-7.19 (m 2H), 6.95-6.91 (m, 2H), 6.83-6.81 (m, 1H), 3.35-3.31 (m, 12H), 3.25-3.24 (m 2H), 2.80-2.75 (m, 2H), 1.93 (s, 3H), 1.34-1.29 (m, 2H), 0.91-0.87 (m, 2H).

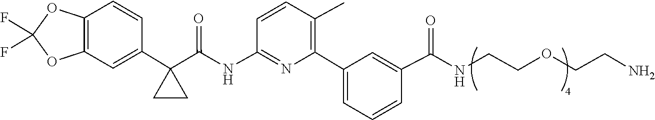

Linker 112: N-(14-amino-3,6,9,12-tetraoxatetradecyl)-3-(6-(1-(2,2-difluorobenzo[d][1,3]dioxol-5-yl)cyclopropane-1-carboxamido)-3-methylpyridin-2-yl)benzamide White solid, 58% yield. ¹H NMR (400 MHz, CD₃OD) δ 7.72 (d, J=8.0 Hz, 1H), 7.62-7.57 (m, 3H), 7.27-7.26 (m, 2H), 6.99-6.94 (m, 2H), 6.85-6.83 (m, 1H), 3.39-3.30 (m, 18H), 2.85-2.82 (m, 2H), 1.96 (s, 3H), 1.37-1.33 (m, 2H), 0.95-0.90 (m, 2H).

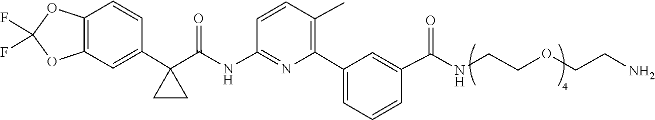

Linker 113: N-(17-amino-3,6,9,12,15-pentaoxaheptadecyl)-3-(6-(1-(2,2-difluorobenzo[d][1,3]dioxol-5-yl)cyclopropane-1-carboxamido)-3-methylpyridin-2-yl)benzamide White solid, 63% yield. ¹H NMR (400 MHz, CD₃OD) δ 7.85 (d, J=8.0 Hz, 1H), 7.73-7.63 (m, 3H), 7.37-7.33 (m, 2H), 7.11-7.06 (m, 2H), 6.98-6.96 (m, 1H), 3.53-3.48 (m, 2H), 3.45-3.40 (m, 20H), 2.96-2.90 (m, 2H), 2.06 (s, 3H), 1.48-1.44 (m, 2H), 1.05-1.00 (m, 2H).

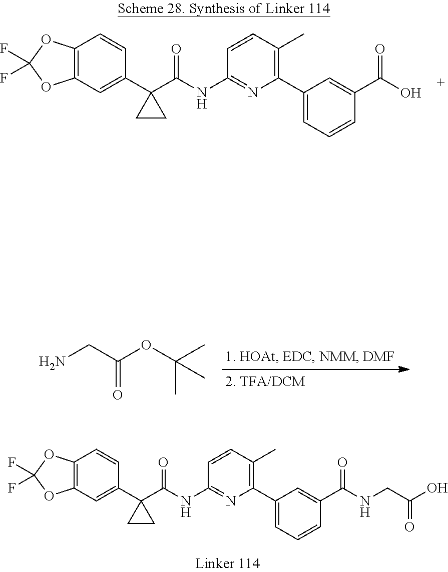

Linker 114: (3-(6-(1-(2,2-difluorobenzo[d1][1,3]dioxol-5-yl)cyclopropane-1-carboxamido)-3-methylpyridin-2-yl)benzoyl)glycine 3-(6-(1-(2,2-difluorobenzo[d][1,3]dioxol-5-yl)cyclopropane-1-carboxamido)-3-methylpyridin-2-yl)benzoic acid (100 mg, 0.22 mmol, 1.0 eq), tert-butyl glycinate (31 mg, 0.24 mmol, 1.1 eq) EDC-HCl (63 mg, 0.33 mmol, 1.5 eq), HOAt (45 mg, 0.33 mmol, 1.5 eq), NMM (67 mg, 0.66 mmol, 3.0 eq) were stirred in 1.5 mL DMF at room temperature for 2 h. Then the mixture was purified via RP-C18 column [MeOH/Water (0.1% TFA)]. Then the product was dissolved in 1 mL DCM and 1 mL TFA, the mixture was stirred at room temperature for 1 h. Then removed all the volatiles and purified via RP-C18 column [MeOH/Water (0.1% TFA)]. The product was obtained as a white solid (70 mg, 61% yield over two steps). 1H NMR (400 MHz, CD₃OD) δ 8.08 (d, J=8.5 Hz, 1H), 7.96 (d, J=6.6 Hz, 1H), 7.92 (s, 1H), 7.84 (d, J=8.5 Hz, 1H), 7.64-7.57 (dt, J=6.4, 1.6 Hz, 2H), 7.39 (s, 1H), 7.34 (d, J=8.3 Hz, 1H), 7.23 (d, J=8.3 Hz, 1H), 4.14-4.06 (m, 2H), 2.32-2.26 (s, 3H), 1.75-1.68 (m, 2H), 1.32-1.23 (m, 2H).

Linkers 115-125 are synthesized following the same procedure for preparing linker 114.

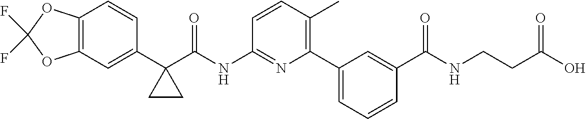 [SMILES: Cc1ccc(NC(=O)C2(c3ccc4c(c3)OC(F)(F)O4)CC2)nc1-c1cccc(C(=O)NCCC(=O)O)c1]

Linker 115: 3-(3-(6-(1-(2,2-difluorobenzo[d][1,3]dioxol-5-yl)cyclopropane-1-carboxamido)-3-methylpyridin-2-yl)benzamido)propanoic acid White solid, 51% yield. ¹H NMR (400 MHz, CD₃OD) δ 8.09-7.90 (m, 4H), 7.69-7.60 (m, 2H), 7.40 (s, 1H), 7.35 (d, J=8.4 Hz, 1H), 7.25 (d, J=8.3 Hz, 1H), 3.71-3.63 (m, 2H), 2.66 (t, J=7.3 Hz, 2H), 2.33 (s, 3H), 1.76-1.69 (m, 2H), 1.36-1.27 (m, 2H).

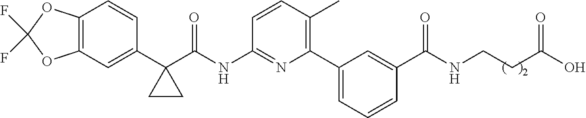

Linker 116: 4-(3-(6-(1-(2,2-difluorobenzo[d][1,3]dioxol-5-yl)cyclopropane-1-carboxamido)-3-methylpyridin-2-yl)benzamido)butanoic acid White solid, 63% yield. ¹H NMR (400 MHz, CD₃OD) δ 7.96 (d, J=8.5 Hz, 1H), 7.83-7.76 (m, 3H), 7.54-7.43 (m, 2H), 7.27 (s, 1H), 7.22 (d, J=8.3 Hz, 1H), 7.11 (d, J=8.3 Hz, 1H), 3.33 (t, J=6.9 Hz, 2H), 2.28 (t, J=7.3 Hz, 2H), 2.18 (s, 3H), 1.85-1.79 (m, 2H), 1.63-1.57 (m, 2H), 1.20-1.12 (m, 2H).

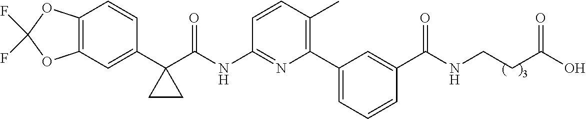

Linker 117: 5-(3-(6-(1-(2,2-difluorobenzo[d][1,3]dioxol-5-yl)cyclopropane-1-carboxamido)-3-methylpyridin-2-yl)benzamido)pentanoic acid White solid, 63% yield. ¹H NMR (400 MHz, CD₃OD) δ 8.08 (d, J=8.6 Hz, 1H), 7.94-7.84 (m, 3H), 7.62-7.57 (m, 2H), 7.40 (s, 1H), 7.34 (d, J=10.4 Hz, 1H), 7.24 (d, J=8.4 Hz, 1H), 3.47-3.35 (m, 2H), 2.39-2.32 (m, 2H), 2.29 (s, 3H), 1.73-1.63 (m, 6H), 1.33-1.26 (m, 2H).

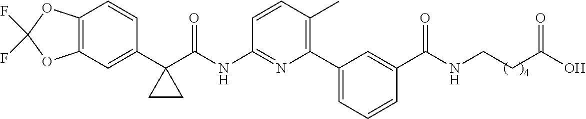

Linker 118: 6-(3-(6-(1-(2,2-difluorobenzo[d][1,3]dioxol-5-yl)cyclopropane-1-carboxamido)-3-methylpyridin-2-yl)benzamido)hexanoic acid White solid, 65% yield. ¹H NMR (400 MHz, CD₃OD) δ 8.06 (d, J=8.5 Hz, 1H), 7.88 (d, J=6.4 Hz, 1H), 7.85-7.80 (m, 2H), 7.56 (d, J=5.2 Hz, 2H), 7.37 (s, 1H), 7.31 (d, J=8.3 Hz, 1H), 7.21 (d, J=8.3 Hz, 1H), 3.38 (t, J=6.3 Hz, 2H), 2.31 (t, J=8.4 Hz, 2H), 2.27 (s, 3H), 1.71-1.58 (m, 6H), 1.48-1.37 (m, 2H), 1.30-1.24 (m, 2H).

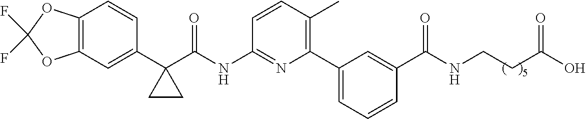

Linker 119: 7-(3-(6-(1-(2,2-difluorobenzo[d][1,3]dioxol-5-yl)cyclopropane-1-carboxamido)-3-methylpyridin-2-yl)benzamido)heptanoic acid White solid, 61% yield. ¹H NMR (400 MHz, CD₃OD) δ 8.05 (d, J=8.5 Hz, 1H), 7.87 (d, J=5.1 Hz, 1H), 7.85-7.79 (m, 2H), 7.59-7.51 (m, 2H), 7.35 (s, 1H), 7.30 (d, J=8.3 Hz, 1H), 7.19 (d, J=8.3 Hz, 1H), 3.38-3.33 (m, 2H), 2.31-2.24 (m, 5H), 1.71-1.64 (m, 2H), 1.64-1.56 (m, 4H), 1.44-1.36 (m, 4H), 1.29-1.22 (m, 2H).

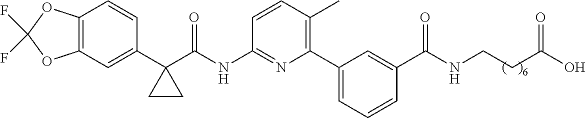

Linker 120: 8-(3-(6-(1-(2,2-difluorobenzo[d][1,3]dioxol-5-yl)cyclopropane-1-carboxamido)-3-methylpyridin-2-yl)benzamido)octanoic acid White solid, 59% yield. ¹H NMR (400 MHz, CD₃OD) δ 8.04 (d, J=8.5 Hz, 1H), 7.86 (d, J=6.0 Hz, 1H), 7.83 (s, 1H), 7.79 (d, J=8.5 Hz, 1H), 7.54 (d, J=5.0 Hz, 2H), 7.34 (s, 1H), 7.29 (d, J=8.1 Hz, 1H), 7.18 (d, J=8.3 Hz, 1H), 3.38-3.33 (m, 2H), 2.30-2.22 (m, 5H), 1.70-1.64 (m, 2H), 1.62-1.53 (m, 4H), 1.42-1.30 (m, 6H), 1.27-1.20 (m, 2H).

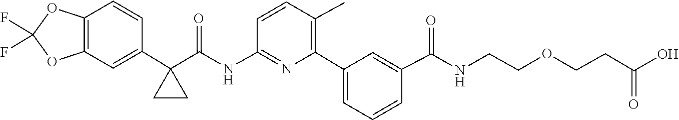 [SMILES: Cc1ccc(NC(=O)C2(c3ccc4c(c3)OC(F)(F)O4)CC2)nc1-c1cccc(C(=O)NCCOCCC(=O)O)c1]

Linker 121: 3-(2-(3-(6-(1-(2,2-difluorobenzo[d][1,3]dioxol-5-yl)cyclopropane-1-carboxamido)-3-methylpyridin-2-yl)benzamido)ethoxy)propanoic acid White solid, 67% yield. ¹H NMR (400 MHz, CD₃OD) δ 8.03 (d, J=8.6 Hz, 1H), 7.88 (d, J=6.8 Hz, 1H), 7.83 (s, 1H), 7.78 (d, J=8.6 Hz, 1H), 7.57-7.49 (m, 2H), 7.32 (s, 1H), 7.28 (d, J=8.3 Hz, 1H), 7.17 (d, J=8.3 Hz, 1H), 3.71 (t, J=6.1 Hz, 2H), 3.64-3.57 (m, 2H), 3.56-3.50 (m, 2H), 2.52 (t, J=6.1 Hz, 2H), 2.24 (s, 3H), 1.67-1.64 (m, 2H), 1.25-1.20 (m, 2H).

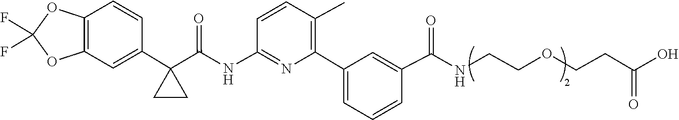

Linker 122: 3-(2-(2-(3-(6-(1-(2,2-difluorobenzo[d][1,3]dioxol-5-yl)cyclopropane-1-carboxamido)-3-methylpyridin-2-yl)benzamido)ethoxy)ethoxy)propanoic acid White solid, 63% yield. ¹H NMR (400 MHz, CD₃OD) δ 8.05 (d, J=8.8 Hz, 1H), 7.93-7.89 (m, 1H), 7.86 (s, 1H), 7.83 (d, J=7.9 Hz, 1H), 7.60-7.53 (m, 2H), 7.36 (s, 1H), 7.31 (d, J=8.2 Hz, 1H), 7.21 (d, J=8.3 Hz, 1H), 3.71 (d, J=5.5 Hz, 2H), 3.67-3.53 (m, 8H), 2.48 (t, J=5.8 Hz, 2H), 2.26 (s, 3H), 1.70-1.62 (m, 2H), 1.28-1.22 (m, 2H).

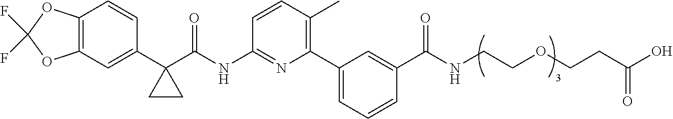

Linker 123: 1-(3-(6-(1-(2,2-difluorobenzo[d][1,3]dioxol-5-yl)cyclopropane-1-carboxamido)-3-methylpyridin-2-yl)phenyl)-1-oxo-5,8,11-trioxa-2-azatetradecan-14-oic acid White solid, 60% yield. ¹H NMR (400 MHz, CD₃OD) δ 8.04 (d, J=7.8 Hz, 1H), 7.95-7.85 (m, 3H), 7.63-7.54 (m, 2H), 7.36 (s, 1H), 7.30 (d, J=9.0 Hz, 1H), 7.20 (d, J=7.4 Hz, 1H), 3.67-3.50 (m, 14H), 2.47 (t, J=6.1 Hz, 2H), 2.27 (s, 3H), 1.72-1.66 (m 2H), 1.30-1.24 (m, 2H).

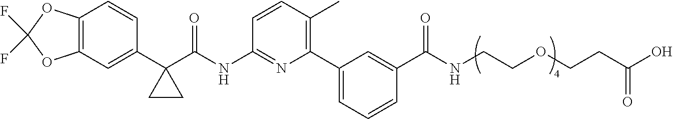

Linker 124: 1-(3-(6-(1-(2,2-difluorobenzo[d][1,3]dioxol-5-yl)cyclopropane-1-carboxamido)-3-methylpyridin-2-yl)phenyl)-1-oxo-5,8,11,14-tetraoxa-2-azaheptadecan-17-oic acid White solid, 65% yield. ¹H NMR (400 MHz, CD₃OD) δ 8.04-7.97 (m, 2H), 7.97-7.93 (m, 1H), 7.91 (s, 1H), 7.65-7.56 (m, 2H), 7.36 (s, 1H), 7.30 (d, J=7.9 Hz, 1H), 7.20 (d, J=8.1 Hz, 1H), 3.68-3.60 (m, 8H), 3.59-3.50 (m, 10H), 2.48 (t, J=6.1 Hz, 2H), 2.29 (s, 3H), 1.74-1.67 (m,

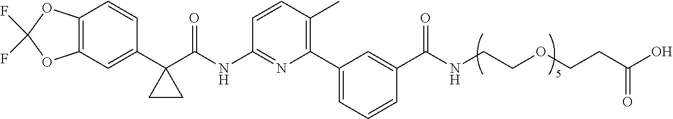

Linker 125: 1-(3-(6-(1-(2,2-difluorobenzo[d][1,3]dioxol-5-yl)cyclopropane-1-carboxamido)-3-methylpyridin-2-yl)phenyl)-1-oxo-5,8,11,14,17-pentaoxa-2-azaicosan-20-oic acid White solid, 61% yield. ¹H NMR (400 MHz, CD₃OD) δ 7.97 (s, 2H), 7.91 (d, J=6.9 Hz, 1H), 7.87 (s, 1H), 7.63-7.53 (m, 2H), 7.31 (s, 1H), 7.25 (d, J=8.2 Hz, 1H), 7.16 (d, J=8.3 Hz, 1H), 3.65-3.56 (m, 8H), 3.55-3.47 (m, 14H), 2.45 (t, J=6.0 Hz, 2H), 2.25 (s, 3H), 1.69-1.63 (m, 2H), 1.27-1.21 (m, 2H).

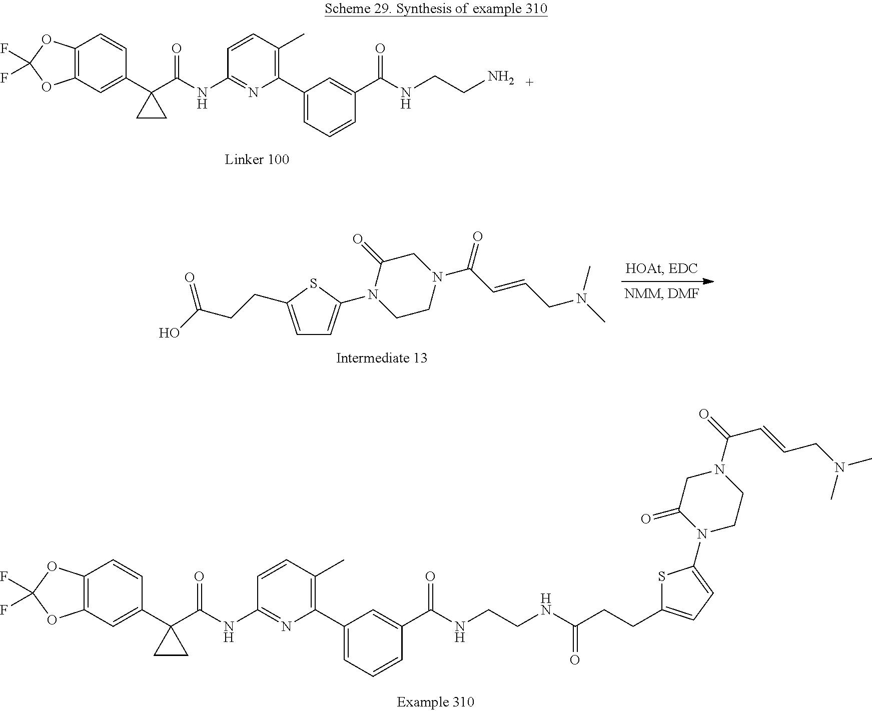 [SMILES: Cc1ccc(NC(=O)C2(c3ccc4c(c3)OC(F)(F)O4)CC2)nc1-c1cccc(C(=O)NCCN)c1 ; CN(C)C/C=C/C(=O)N1CCN(c2ccc(CCC(=O)O)s2)C(=O)C1 ; Cc1ccc(NC(=O)C2(c3ccc4c(c3)OC(F)(F)O4)CC2)nc1-c1cccc(C(=O)NCCNC(=O)CCc2ccc(N3CCN(C(=O)/C=C/CN(C)C)CC3=O)s2)c1]

Example 310 (E)-3-(6-(1-(2,2-difluorobenzo[d][1,3]dioxol-5-yl)cyclopropane-1-carboxamido)-3-methylpyridin-2-yl)-N-(2-(3-(5-(4-(4-(dimethylamino)but-2-enoyl)-2-oxopiperazin-1-yl)thiophen-2-yl)propanamido)ethyl)benzamide Linker 100 (10 mg, 0.02 mmol, 1.0 eq), intermediate 13 (8 mg, 0.022 mmol, 1.1 eq), EDC HCl (5.7 mg, 0.03 mmol, 1.5 eq), HOAt (4.1 mg, 0.03 mmol, 1.5 eq), NMM (6.0 mg, 0.06 mmol, 3.0 eq) were stirred in 1.5 mL DMF at room temperature for 2 h. Then the mixture was purified via Prep-HPLC to yield titled compound as a white solid (7.8 mg, 46%). ¹H NMR (400 MHz, CD₃OD) δ 8.04 (d, J=8.4 Hz, 1H), 7.87-7.77 (m, 3H), 7.61-7.52 (m, 2H), 7.35 (s, 1H), 7.30 (d, J=8.1 Hz, 1H), 7.19 (d, J=7.4 Hz, 1H), 7.00-6.70 (m, 2H), 6.58-6.51 (m, 2H), 4.44 (s, 1H), 4.36 (s, 1H), 4.02-3.90 (m, 4H), 3.84 (s, 1H), 3.74 (s, 1H), 3.48-3.36 (m, 4H), 3.05-2.96 (m, 2H), 2.89 (s, 6H), 2.53-2.46 (m, 2H), 2.25 (s, 3H), 1.74-1.69 (m, 2H), 1.32-1.26 (m, 2H). MS (ESI) [M+H]⁺=842.1.

Examples 311-323 are synthesized as the same procedure for preparing example 310 from related linkers and intermediate 13.

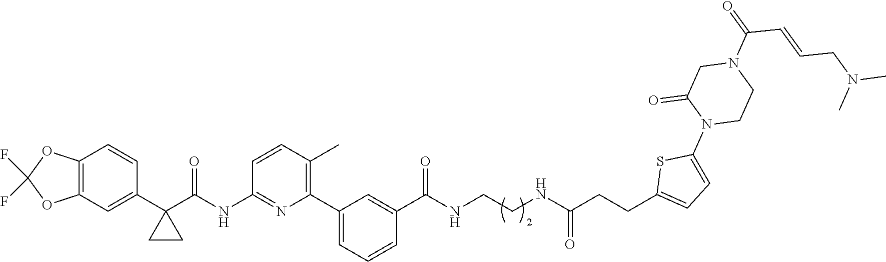

Example 311: (E)-3-(6-(1-(2,2-difluorobenzo[d][1,3]dioxol-5-yl)cyclopropane-1-carboxamido)-3-methylpyridin-2-yl)-N-(3-(3-(5-(4-(4-(dimethylamino)but-2-enoyl)-2-oxopiperazin-1-yl)thiophen-2-yl)propanamido)propyl)benzamide White solid, 45% yield. ¹H NMR (400 MHz, CD₃OD) δ 8.06 (d, J=7.9 Hz, 1H), 7.94-7.83 (m, 3H), 7.63-7.54 (m, 2H), 7.37 (s, 1H), 7.31 (d, J=8.2 Hz, 1H), 7.21 (d, J=7.8 Hz, 1H), 6.92 (dd, J=39.4, 15.1 Hz, 1H), 6.80-7.70 (m, 1H), 6.70-6.62 (m, 2H), 4.46 (s, 1H), 4.38 (s, 1H), 4.06-3.82 (m, 6H), 3.27-3.20 (m, 2H), 3.09-3.02 (m, 2H), 2.99-2.82 (m, 8H), 2.56-2.49 (m, 2H), 2.28 (s, 3H), 1.78-1.65 (m, 4H), 1.32-1.26 (m, 2H). MS (ESI) [M+H]⁺=856.4.

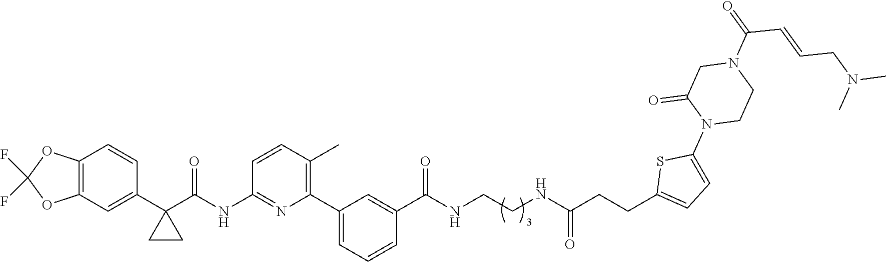

Example 312: (E)-3-(6-(1-(2,2-difluorobenzo[d][1,3]dioxol-5-yl)cyclopropane-1-carboxamido)-3-methylpyridin-2-yl)-N-(4-(3-(5-(4-(4-(dimethylamino)but-2-enoyl)-2-oxopiperazin-1-yl)thiophen-2-yl)propanamido)butyl)benzamide White solid, 40% yield. 1H NMR (400 MHz, CD₃OD) δ 8.04 (d, J=7.5 Hz, 1H), 7.91-7.83 (m, 3H), 7.60-7.53 (m, 2H), 7.35 (s, 1H), 7.30 (d, J=8.9 Hz, 1H), 7.19 (d, J=8.1 Hz, 1H), 6.91 (dd, J=38.0, 15.1 Hz, 1H), 6.79-6.69 (m, 1H), 6.62-6.59 (m, 2H), 4.46 (s, 1H), 4.38 (s, 1H), 4.06-3.80 (m, 6H), 3.36-3.31 (m, 2H), 3.21-3.13 (m, 2H), 3.06-2.97 (m, 2H), 2.89 (s, 6H), 2.51-2.44 (m, 2H), 2.26 (s, 3H), 1.74-1.69 (m, 2H), 1.59-1.52 (m, 4H), 1.32-1.26 (m, 2H). MS (ESI) [M+H]⁺=870.5.

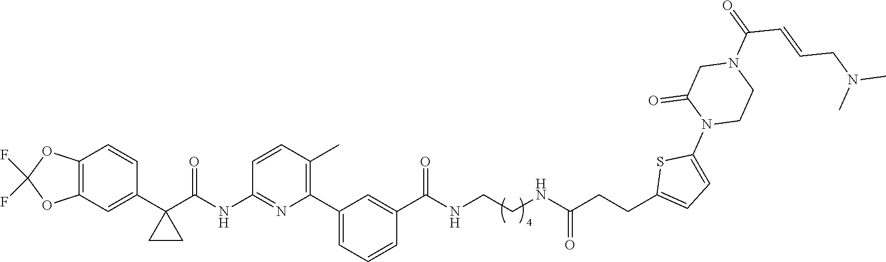

Example 313: (E)-3-(6-(1-(2,2-difluorobenzo[d][1,3]dioxol-5-yl)cyclopropane-1-carboxamido)-3-methylpyridin-2-yl)-N-(5-(3-(5-(4-(4-(dimethylamino)but-2-enoyl)-2-oxopiperazin-1-yl)thiophen-2-yl)propanamido)pentyl)benzamide White solid, 45% yield. ¹H NMR (400 MHz, CD₃OD) δ 8.03 (d, J=8.2 Hz, 1H), 7.90-7.82 (m, 3H), 7.59-7.52 (m, 2H), 7.35 (s, 1H), 7.29 (d, J=8.0 Hz, 1H), 7.19 (d, J=7.4 Hz, 1H), 6.92 (dd, J=36.2, 15.2 Hz, 1H), 6.79-7.69 (m, 1H), 6.69-6.60 (m, 2H), 4.47 (s, 1H), 4.39 (s, 1H), 4.10-4.02 (m, 2H), 3.97-3.80 (m, 4H), 3.35-3.30 (m, 2H), 3.17-3.11(m, 2H), 3.03-2.96 (m, 2H), 2.89 (s, 6H), 2.45 (t, J=7.5 Hz, 2H), 2.25 (s, 3H), 1.69-1.65 (m, 2H), 1.62-1.53 (m, 2H), 1.52-1.42 (m, 2H), 1.36-1.20 (m, 4H). MS (ESI) [M+H]⁺==884.4.

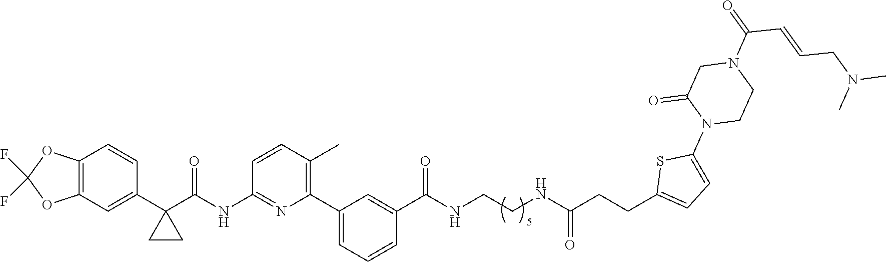

Example 314: (E)-3-(6-(1-(2,2-difluorobenzo[d][1,3]dioxol-5-yl)cyclopropane-1-carboxamido)-3-methylpyridin-2-yl)-N-(6-(3-(5-(4-(4-(dimethylamino)but-2-enoyl)-2-oxopiperazin-1-yl)thiophen-2-yl)propanamido)hexyl)benzamide White solid, 44% yield. ¹H NMR (400 MHz, CD₃OD) δ 8.04 (d, J=8.4 Hz, 1H), 7.91-7.83 (m, 3H), 7.60-7.53 (m, 2H), 7.35 (s, 1H), 7.30 (d, J=8.1 Hz, 1H), 7.20 (d, J=7.5 Hz, 1H), 6.93 (dd, J=36.6, 15.1 Hz, 1H), 6.81-6.69 (m, 1H), 6.66-6.60 (m, 2H), 4.48 (s, 1H), 4.39 (s, 1H), 4.16-3.77 (m, 6H), 3.37-3.31 (m, 2H), 3.17-3.10 (m, 2H), 3.04-2.96 (m, 2H), 2.90 (s, 6H), 2.51-2.44 (m, 2H), 2.26 (s, 3H), 1.70-1.65 (m, 2H), 1.62-1.40 (m, 4H), 1.38-1.23 (m, 6H). MS (ESI) [M+H]⁻=898.5.

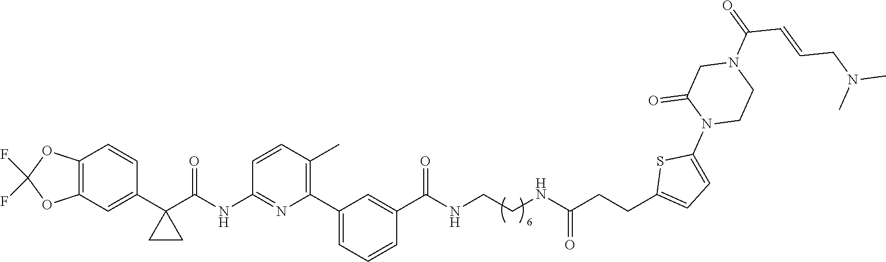

Example 315: (E)-3-(6-(1-(2,2-difluorobenzo[d][1,3]dioxol-5-yl)cyclopropane-1-carboxamido)-3-methylpyridin-2-yl)-N-(7-(3-(5-(4-(4-(dimethylamino)but-2-enoyl)-2-oxopiperazin-1-yl)thiophen-2-yl)propanamido)heptyl)benzamide White solid, 47% yield. ¹H NMR (400 MHz, CD₃OD) δ 8.04 (d, J=8.4 Hz, 1H), 7.91-7.80 (m, 3H), 7.60-7.53 (m, 2H), 7.35 (s, 1H), 7.30 (d, J=8.1 Hz, 1H), 7.19 (d, J=8.1 Hz, 1H), 6.94 (dd, J=37.3, 15.1 Hz, 1H), 6.81-6.69 (m, 1H), 6.65-6.60 (m, 2H), 4.49 (s, 1H), 4.41 (s, 1H), 4.03-3.81 (m, 6H), 3.37-3.31 (m, 2H), 3.16-3.09 (m, 2H), 3.01 (t, J=6.6 Hz, 2H), 2.90 (s, 6H), 2.51-2.44 (m, 2H), 2.25 (s, 3H), 1.67-1.54 (m, 4H), 1.49-1.21 (m, 10H). MS (ESI) [M+H]⁺=912.6.

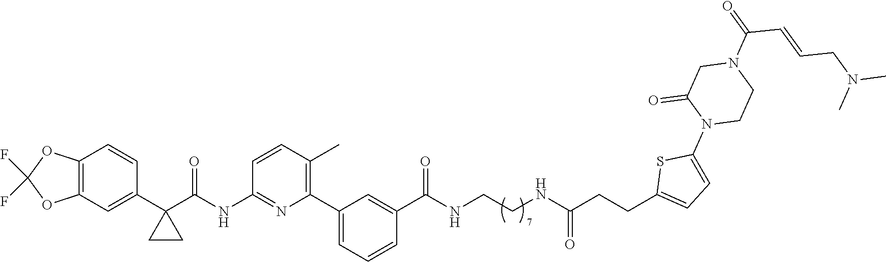

Example 316: (E)-3-(6-(1-(2,2-difluorobenzo[d][1,3]dioxol-5-yl)cyclopropane-1-carboxamido)-3-methylpyridin-2-yl)-N-(8-(3-(5-(4-(4-(dimethylamino)but-2-enoyl)-2-oxopiperazin-1-yl)thiophen-2-yl)propanamido)octyl)benzamide White solid, 43% yield. ¹H NMR (400 MHz, CD₃OD) δ 8.04 (d, J=8.4 Hz, 1H), 7.91-7.80 (m, 3H), 7.60-7.52 (m, 2H), 7.36 (s, 1H), 7.30 (d, J=8.1 Hz, 1H), 7.20 (d, J=8.1 Hz, 1H), 6.94 (dd, J=37.3, 15.1 Hz, 1H), 6.81-6.70 (m, 1H), 6.65-6.60 (m, 2H), 4.49 (s, 1H), 4.41 (s, 1H), 4.05-3.81 (m, 6H), 3.40-3.32 (m, 2H), 3.18-3.08 (m, 2H), 3.01 (t, J=6.6 Hz, 2H), 2.90 (s, 6H), 2.51-2.44 (m, 2H), 2.25 (s, 3H), 1.69-1.54 (m, 4H), 1.49-1.21 (m, 12H). MS (ESI) [M+H]<=926.5.

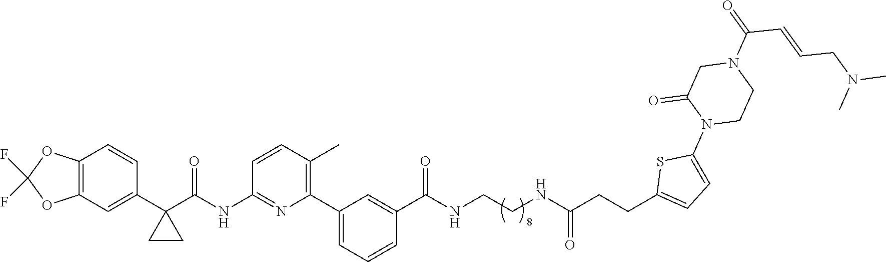

Example 317: (E)-3-(6-(1-(2,2-difluorobenzo[d][1,3]dioxol-5-yl)cyclopropane-1-carboxamido)-3-methylpyridin-2-yl)-N-(9-(3-(5-(4-(4-(dimethylamino)but-2-enoyl)-2-oxopiperazin-1-yl)thiophen-2-yl)propanamido)nonyl)benzamide White solid, 48% yield. 1H NMR (400 MHz, CD₃OD) δ 8.05 (d, J=8.3 Hz, 1H), 7.90-7.81 (m, 3H), 7.60-7.52 (m, 2H), 7.36 (s, 1H), 7.31 (d, J=8.2 Hz, 1H), 7.20 (d, J=7.8 Hz, 1H), 6.94 (dd, J=38.1, 15.1 Hz, 1H), 6.81-6.71 (m, 1H), 6.63 (s, 2H), 4.49 (s, 1H), 4.41 (s, 1H), 4.03 (s, 2H), 3.98-3.82 (m, 4H), 3.39-3.33 (m, 2H), 3.14-3.07 (m, 2H), 3.05-2.99 (m, 2H), 2.91 (s, 6H), 2.52-2.45 (m, 2H), 2.26 (s, 3H), 1.70-1.55 (m, 4H), 1.46-1.22 (m, 14H). MS (ESI) [M+H]⁺=940.6.

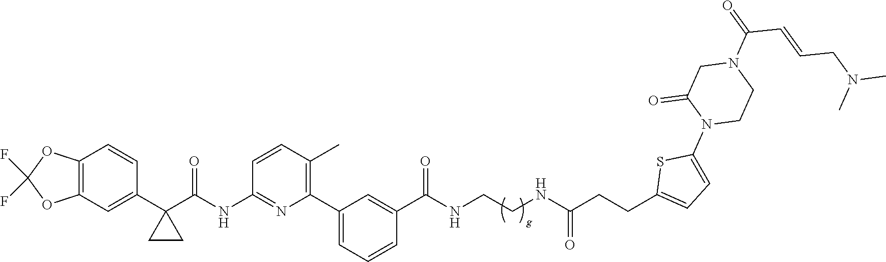

Example 318: (E)-3-(6-(1-(2,2-difluorobenzo[d][1,3]dioxol-5-yl)cyclopropane-1-carboxamido)-3-methylpyridin-2-yl)-N-(10-(3-(5-(4-(4-(dimethylamino)but-2-enoyl)-2-oxopiperazin-1-yl)thiophen-2-yl)propanamido)decyl)benzamide White solid, 45% yield. ¹H NMR (400 MHz, CD₃OD) δ 8.03 (d, J=8.3 Hz, 1H), 7.91-7.82 (m, 3H), 7.61-7.51 (m, 2H), 7.35 (s, 1H), 7.29 (d, J=8.0 Hz, 1H), 7.19 (d, J=8.1 Hz, 1H), 6.93 (dd, J=38.3, 15.2 Hz, 1H), 6.80-6.71 (m, 1H), 6.61 (s, 2H), 4.48 (s, 1H), 4.40 (s, 1H), 4.03 (s, 2H), 3.97-3.80 (m, 4H), 3.39-3.31 (m, 2H), 3.14-3.06 (m, 2H), 3.04-2.97 (m, 2H), 2.89 (s, 6H), 2.49-2.43 (m, 2H), 2.26 (s, 3H), 1.67 (s, 2H), 1.63-1.54 (m, 2H), 1.43-1.22 (m, 16H). MS (ESI) [M+H]⁺=954.6.

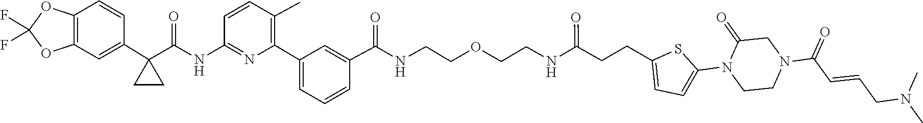 [SMILES: Cc1ccc(NC(=O)C2(c3ccc4c(c3)OC(F)(F)O4)CC2)nc1-c1cccc(C(=O)NCCOCCNC(=O)CCc2ccc(N3CCN(C(=O)/C=C/CN(C)C)CC3=O)s2)c1]

Example 319: (E)-3-(6-(1-(2,2-difluorobenzo[d][1,3]dioxol-5-yl)cyclopropane-1-carboxamido)-3-methylpyridin-2-yl)-N-(2-(2-(3-(5-(4-(4-(dimethylamino)but-2-enoyl)-2-oxopiperazin-1-yl)thiophen-2-yl)propanamido)ethoxy)ethyl)benzamide White solid, 46% yield. ¹H NMR (400 MHz, CD₃OD) δ 8.04 (d, J=8.1 Hz, 1H), 7.90-7.78 (m, 3H), 7.60-7.51 (m, 2H), 7.37 (s, 1H), 7.31 (d, J=8.2 Hz, 1H), 7.20 (d, J=7.9 Hz, 1H), 6.94 (dd, J=34.7, 15.1 Hz, 1H), 6.80-6.71 (m, 1H), 6.57-6.53 (m, 2H), 4.48 (s, 1H), 4.40 (s, 1H), 4.08-3.76 (m, 6H), 3.62-3.48 (m, 6H), 3.37-3.32 (m, 2H), 2.98-2.88 (m, 8H), 2.45-2.36 (m, 2H), 2.25 (s, 3H), 1.71-1.65 (m, 2H), 1.27-1.23 (m, 2H). MS (ESI) [M+H]⁺=886.5.

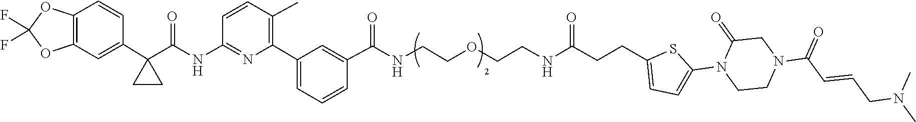

Example 320: (E)-3-(6-(1-(2,2-difluorobenzo[d][1,3]dioxol-5-yl)cyclopropane-1-carboxamido)-3-methylpyridin-2-yl)-N-(2-(2-(2-(3-(5-(4-(4-(dimethylamino)but-2-enoyl)-2-oxopiperazin-1-yl)thiophen-2-yl)propanamido)ethoxy)ethoxy)ethyl)benzamide White solid, 44% yield. ¹H NMR (400 MHz, CD₃OD) δ 8.02 (d, J=8.0 Hz, 1H), 7.91-7.83 (m, 3H), 7.61-7.51 (m, 2H), 7.34 (s, 1H), 7.29 (d, J=8.2 Hz, 1H), 7.19 (d, J=8.2 Hz, 1H), 6.92 (dd, J=37.3, 15.1 Hz, 1H), 6.80-6.69 (m, 1H), 6.61-6.57 (m, 2H), 4.47 (s, 1H), 4.39 (s, 1H), 4.07-3.78 (m, 6H), 3.65-3.51 (m, 8H), 3.47-3.42 (m, 2H), 3.27-3.22 (m, 2H), 3.01-2.93 (m, 2H), 2.89 (s, 6H), 2.47-2.40 (m, 2H), 2.25 (s, 3H), 1.71-1.65 (m, 2H), 1.27-1.23 (m, 2H). MS (ESI) [M+H]⁺=930.6.

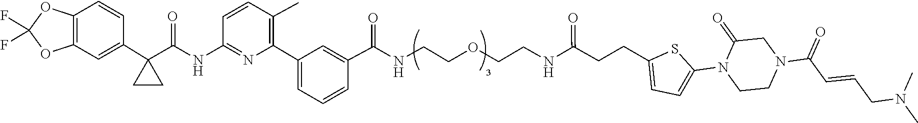

Example 321: (E)-3-(6-(1-(2,2-difluorobenzo[d][1,3]dioxol-5-yl)cyclopropane-1-carboxamido)-3-methylpyridin-2-yl)-N-(15-(5-(4-(4-(dimethylamino)but-2-enoyl)-2-oxopiperazin-1-yl)thiophen-2-yl)-13-oxo-3,6,9-trioxa-12-azapentadecyl)benzamide White solid, 43% yield. ¹H NMR (400 MHz, CD₃OD) δ 8.03 (d, J=7.4 Hz, 1H), 7.91-7.82 (m, 3H), 7.60-7.51 (m, 2H), 7.35 (s, 1H), 7.30 (d, J=8.1 Hz, 1H), 7.19 (d, J=7.7 Hz, 1H), 6.92 (dd, J=37.9, 15.2 Hz, 1H), 6.80-6.69 (m, 1H), 6.64-6.56 (m, 2H), 4.47 (s, 1H), 4.39 (s, 1H), 4.04-3.79 (m, 6H), 3.67-3.47 (m, 12H), 3.44-3.38 (m, 2H), 3.27-3.22 (m, 2H), 3.02-2.95 (m, 2H), 2.90 (s, 6H), 2.49-2.42 (m, 2H), 2.25 (s, 3H), 1.71-1.65 (m, 2H), 1.27-1.23 (m, 2H). MS (ESI) [M+H]⁺=974.5.

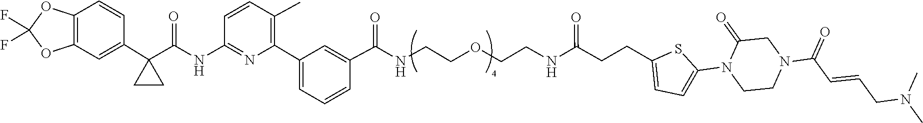

Example 322: (E)-3-(6-(1-(2,2-difluorobenzo[d][1,3]dioxol-5-yl)cyclopropane-1-carboxamido)-3-methylpyridin-2-yl)-N-(18-(5-(4-(4-(dimethylamino)but-2-enoyl)-2-oxopiperazin-1-yl)thiophen-2-yl)-16-oxo-3,6,9,12-tetraoxa-15-azaoctadecyl)benzamide White solid, 45% yield. ¹H NMR (400 MHz, CD₃OD) δ 8.06 (d, J=8.4 Hz, 1H), 7.92-7.80 (m, 3H), 7.61-7.53 (m, 2H), 7.37 (s, 1H), 7.32 (d, J=8.2 Hz, 1H), 7.21 (d, J=7.9 Hz, 1H), 6.94 (dd, J=37.3, 15.2 Hz, 1H), 6.83-6.71 (m, 1H), 6.65-6.58 (m, 2H), 4.49 (s, 1H), 4.42 (s, 1H), 4.09-3.80 (m, 6H), 3.66-3.42 (m, 18H), 3.30-3.27 (m, 2H), 3.06-2.98 (m, 2H), 2.91 (s, 6H), 2.54-2.47 (m, 2H), 2.26 (s, 3H), 1.71-1.65 (m, 2H), 1.27-1.23 (m, 2H). MS (ESI) [M+H]⁺=1018.4.

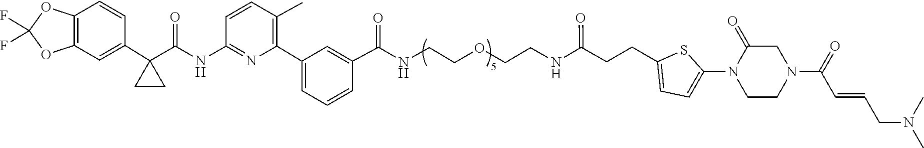

Example 323: (E)-3-(6-(1-(2,2-difluorobenzo[d][1,3]dioxol-5-yl)cyclopropane-1-carboxamido)-3-methylpyridin-2-yl)-N-(21-(5-(4-(4-(dimethylamino)but-2-enoyl)-2-oxopiperazin-1-yl)thiophen-2-yl)-19-oxo-3,6,9,12,15-pentaoxa-18-azahenicosyl)benzamide White solid, 47% yield. ¹H NMR (400 MHz, CD₃OD) δ 8.09 (d, J=8.1 Hz, 1H), 7.91-7.83 (m, 2H), 7.75 (d, J=8.1 Hz, 1H), 7.56 (s, 2H), 7.39 (s, 1H), 7.34 (d, J=8.1 Hz, 1H), 7.23 (d, J=8.1 Hz, 1H), 6.96 (dd, J=36.4, 15.1 Hz, 1H), 6.85-6.72 (m, 1H), 6.68-6.60 (m, 2H), 4.51 (s, 1H), 4.44 (s, 1H), 4.08-3.85 (m, 6H), 3.70-3.52 (m, 20H), 3.52-3.46 (m, 2H), 3.36-3.33 (m, 2H), 3.09-3.00 (m, 2H), 2.93 (s, 6H), 2.56-2.49 (m, 2H), 2.26 (s, 3H), 1.71-1.65 (m, 2H), 1.27-1.23 (m, 2H). MS (ESI) [M+H]⁺=1062.6.

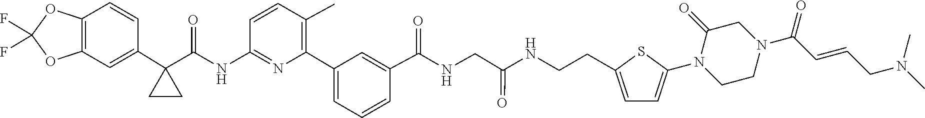 [SMILES: Cc1ccc(NC(=O)C2(c3ccc4c(c3)OC(F)(F)O4)CC2)nc1-c1cccc(C(=O)NCC(=O)NCCc2ccc(N3CCN(C(=O)/C=C/CN(C)C)CC3=O)s2)c1]

Example 324: (E)-3-(6-(1-(2,2-difluorobenzo[d][1,3]dioxol-5-yl)cyclopropane-1-carboxamido)-3-methylpyridin-2-yl)-N-(2-((2-(5-(4-(4-(dimethylamino)but-2-enoyl)-2-oxopiperazin-1-yl)thiophen-2-yl)ethyl)amino)-2-oxoethyl)benzamide Example 324 was synthesized following similar procedure for preparing example 310 from intermediate 15 and linker 114. White solid, 43% yield. ¹H NMR (400 MHz, CD₃OD) δ 7.99 (d, J=7.9 Hz, 1H), 7.91-7.73 (m, 3H), 7.57-7.47 (m, 2H), 7.30 (s, 1H), 7.24 (d, J=8.4 Hz, 1H), 7.13 (d, J=7.4 Hz, 1H), 6.86 (dd, J=34.7, 16.4 Hz, 1H), 6.74-6.64 (m, 1H), 6.62-6.55 (m, 2H), 4.42-4.39 (m, 1H), 4.35-4.31 (m, 1H), 3.98-3.73 (m, 7H), 3.58-3.54 (m, 1H), 3.41-3.34 (m, 2H), 2.91-2.76 (m, 8H), 2.20 (s, 3H), 1.63-1.59 (m, 2H), 1.21-1.17 (m, 2H). MS (ESI) [M+H]⁺=828.3.

Example 325-335 were synthesized following the similar procedure for preparing example 324.

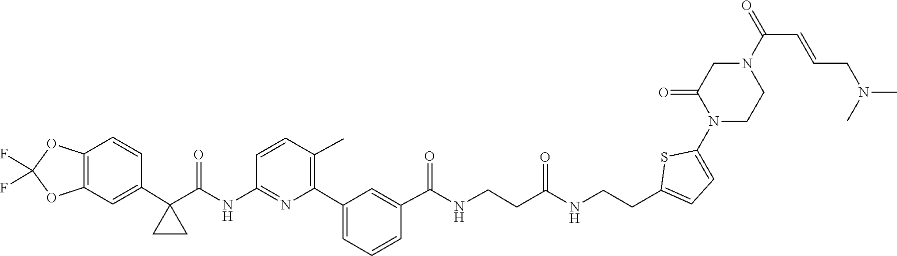 [SMILES: Cc1ccc(NC(=O)C2(c3ccc4c(c3)OC(F)(F)O4)CC2)nc1-c1cccc(C(=O)NCCC(=O)NCCc2ccc(N3CCN(C(=O)/C=C/CN(C)C)CC3=O)s2)c1]

Example 325: (E)-3-(6-(1-(2,2-difluorobenzo[d][1,3]dioxol-5-yl)cyclopropane-1-carboxamido)-3-methylpyridin-2-yl)-N-(3-((2-(5-(4-(4-(dimethylamino)but-2-enoyl)-2-oxopiperazin-1-yl)thiophen-2-yl)ethyl)amino)-3-oxopropyl)benzamide White solid, 42% yield. ¹H NMR (400 MHz, CD₃OD) δ 8.07 (d, J=7.3 Hz, 1H), 7.91-7.78 (m, 3H), 7.60-7.54 (m, 2H), 7.38 (s, 1H), 7.33 (d, J=6.8 Hz, 1H), 7.22 (d, J=7.4 Hz, 1H), 6.94 (dd, J=35.9, 15.3 Hz, 1H), 6.82-6.71 (m, 1H), 6.65-6.61 (m, 2H), 4.49 (s, 1H), 4.42 (s, 1H), 4.05-3.82 (m, 6H), 3.67-3.59 (m, 2H), 3.48-3.39 (m, 2H), 2.95-2.90 (m, 8H), 2.55-2.46 (m, 2H), 2.26 (s, 3H), 1.71-1.69 (m, 2H), 1.29-1.25 (m, 2H). MS (ESI) [M+H]⁺=842.4.

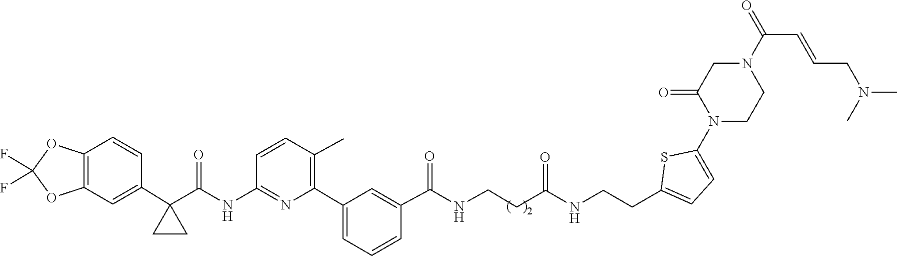

Example 326: (E)-3-(6-(1-(2,2-difluorobenzo[d][1,3]dioxol-5-yl)cyclopropane-1-carboxamido)-3-methylpyridin-2-yl)-N-(4-((2-(5-(4-(4-(dimethylamino)but-2-enoyl)-2-oxopiperazin-1-yl)thiophen-2-yl)ethyl)amino)-4-oxobutyl)benzamide White solid, 45% yield. ¹H NMR (400 MHz, CD₃OD) δ 8.09 (d, J=7.2 Hz, 1H), 7.95-7.79 (m, 3H), 7.60 (s, 2H), 7.40 (s, 1H), 7.35 (d, J=8.1 Hz, 1H), 7.24 (d, J=7.6 Hz, 1H), 6.96 (dd, J=30.3, 15.3 Hz, 1H), 6.84-6.72 (m, 1H), 6.65-6.61 (m, 2H), 4.50 (s, 1H), 4.42 (s, 1H), 4.08-3.97 (m, 4H), 3.93-3.79 (m, 2H), 3.47-3.37 (m, 4H), 2.98-2.90 (m, 8H), 2.38-2.23 (m, 5H), 1.99-1.86 (m, 2H), 1.72-1.70 (m, 2H), 1.31-1.27 (m, 2H). MS (ESI) [M+H]⁺=856.3.

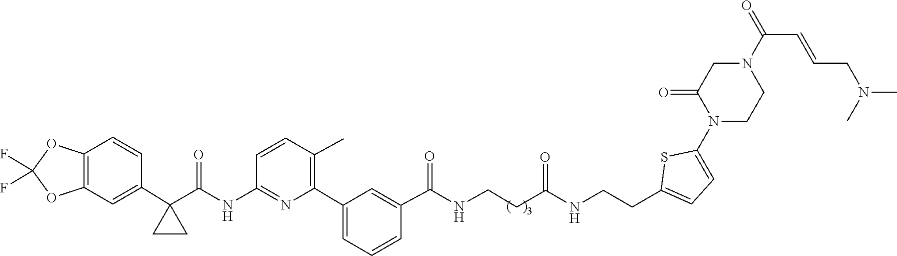

Example 327: (E)-3-(6-(1-(2,2-difluorobenzo[d][1,3]dioxol-5-yl)cyclopropane-1-carboxamido)-3-methylpyridin-2-yl)-N-(5-((2-(5-(4-(4-(dimethylamino)but-2-enoyl)-2-oxopiperazin-1-yl)thiophen-2-yl)ethyl)amino)-5-oxopentyl)benzamide White solid, 46% yield. ¹H NMR (400 MHz, CD₃OD) δ 8.06 (d, 1=8.0 Hz, 1H), 7.93-7.80 (m, 3H), 7.62-7.53 (m, 2H), 7.38 (s, 1H), 7.32 (d, J=7.0 Hz, 1H), 7.22 (d, J=7.4 Hz, 1H), 6.94 (dd, J=37.1, 15.2 Hz, 1H), 6.83-6.70 (m, 1H), 6.68-6.64 (m, 2H), 4.48 (s, 1H), 4.40 (s, 1H), 4.07-3.81 (m, 6H), 3.47-3.34 (m, 4H), 2.94-2.90 (m, 8H), 2.31-2.18 (m, 5H), 1.74-1.55 (m, 6H), 1.29-1.25 (m 2H). MS (ESI [M+H]⁺=870.5.

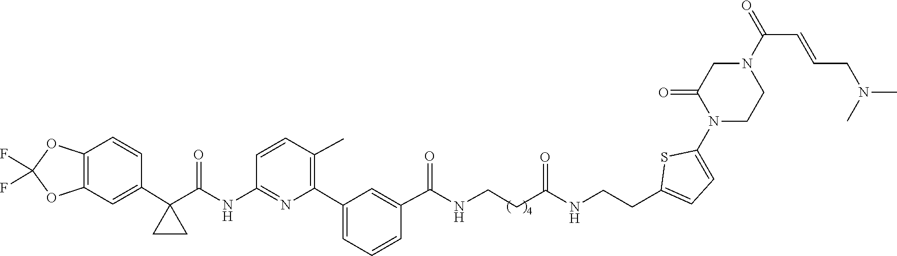

Example 328: (E)-3-(6-(1-(2,2-difluorobenzo[d][1,3]dioxol-5-yl)cyclopropane-1-carboxamido)-3-methylpyridin-2-yl)-N-(6-((2-(5-(4-(4-(dimethylamino)but-2-enoyl)-2-oxopiperazin-1-yl)thiophen-2-yl)ethyl)amino)-6-oxohexyl)benzamide White solid, 42% yield. ¹H NMR (400 MHz, CD₃OD) δ 8.07 (d, J=7.1 Hz, 1H), 7.93-7.76 (m, 3H), 7.57 (s, 2H), 7.40 (s, 1H), 7.34 (d, J=8.1 Hz, 1H), 7.23 (d, J=7.5 Hz, 1H), 6.95 (dd, J=33.8, 15.0 Hz, 1H), 6.83-6.71 (m, 1H), 6.68-6.64 (m, 2H), 4.51 (s, 1H), 4.44 (s, 1H), 4.11-3.83 (m, 6H), 3.43-3.35 (m, 4H), 2.96-2.85 (m, 8H), 2.30-2.15 (m, 5H), 1.74-1.55 (m, 6H), 1.43-1.23 (m, 4H). MS (ESI) [M+H]⁺=884.7.

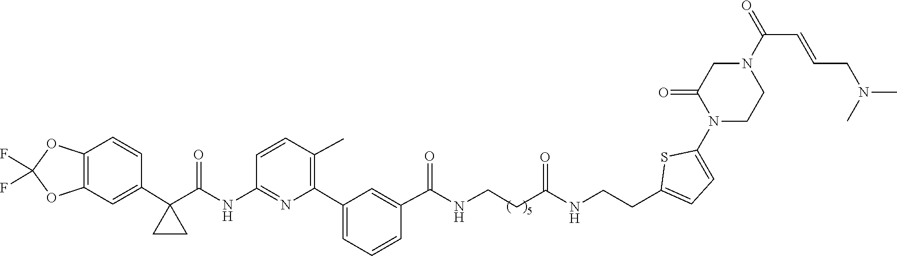

Example 329: (E)-3-(6-(1-(2,2-difluorobenzo[d][1,3]dioxol-5-yl)cyclopropane-1-carboxamido)-3-methylpyridin-2-yl)-N-(7-((2-(5-(4-(4-(dimethylamino)but-2-enoyl)-2-oxopiperazin-1-yl)thiophen-2-yl)ethyl)amino)-7-oxoheptyl)benzamide (MS6178) White solid, 45% yield. ¹H NMR (400 MHz, CD₃OD) δ 8.07 (d, J=7.8 Hz, 1H), 7.94-7.78 (m, 3H), 7.59 (s, 2H), 7.39 (s, 1H), 7.33 (d, J=8.0 Hz, 1H), 7.23 (d, J=8.0 Hz, 1H), 6.95 (dd, J=34.8, 15.2 Hz, 1H), 6.83-6.72 (m, 1H), 6.69-6.66 (m, 2H), 4.51 (s, 1H), 4.44 (s, 1H), 4.11-3.85 (m, 6H), 3.45-3.35 (m, 4H), 2.95-2.90 (m, 8H), 2.27 (s, 3H), 2.21-2.10 (m, 2H), 1.69-1.67 (m, 2H), 1.66-1.56 (m, 4H), 1.39-1.35 (m, 4H), 1.29-1.25 (m, 2H). MS (ESI) [M+H]⁺=898.5.

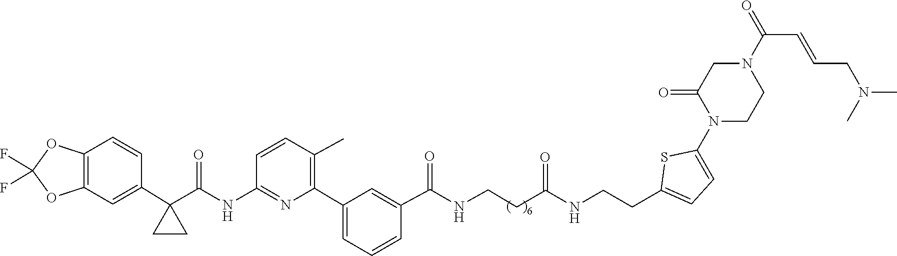

Example 330: (E)-3-(6-(1-(2,2-difluorobenzo[d][1,3]dioxol-5-yl)cyclopropane-1-carboxamido)-3-methylpyridin-2-yl)-N-(8-((2-(5-(4-(4-(dimethylamino)but-2-enoyl)-2-oxopiperazin-1-yl)thiophen-2-yl)ethyl)amino)-8-oxooctyl)benzamide White solid, 43% yield. ¹H NMR (400 MHz, CD₃OD) δ 8.08 (d, J=7.9 Hz, 1H), 7.92-7.77 (m, 3H), 7.58 (s, 2H), 7.39 (s, 1H), 7.34 (d, J=8.1 Hz, 1H), 7.23 (d, J=7.9 Hz, 1H), 6.96 (dd, J=34.2, 15.1 Hz, 1H), 6.83-6.72 (m, 1H), 6.69-6.66 (m, 2H), 4.52 (s, 1H), 4.45 (s, 1H), 4.11-3.87 (m, 6H), 3.45-3.36 (m, 4H), 2.95-2.90 (m, 8H), 2.27 (s, 3H), 2.20-2.14 (m, 2H), 1.74-1.54 (m, 6H), 1.41-1.23 (m, 8H). MS (ESI) [M+H]⁺=912.4.

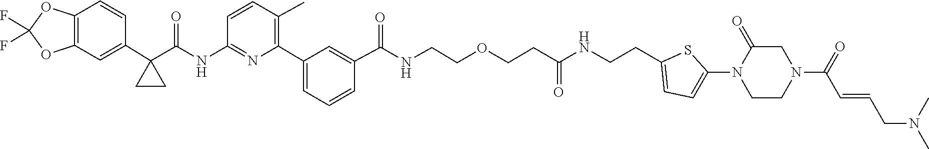 [SMILES: Cc1ccc(NC(=O)C2(c3ccc4c(c3)OC(F)(F)O4)CC2)nc1-c1cccc(C(=O)NCCOCCC(=O)NCCc2ccc(N3CCN(C(=O)/C=C/CN(C)C)CC3=O)s2)c1]

Example 331: (E)-3-(6-(1-(2,2-difluorobenzo[d][1,3]dioxol-5-yl)cyclopropane-1-carboxamido)-3-methylpyridin-2-yl)-N-(2-(3-((2-(5-(4-(4-(dimethylamino)but-2-enoyl)-2-oxopiperazin-1-yl)thiophen-2-yl)ethyl)amino)-3-oxopropoxy)ethyl)benzamide White solid, 41% yield. ¹H NMR (400 MHz, CD₃OD) δ 8.06 (d, J=8.6 Hz, 1H), 7.95-7.75 (m, 3H), 7.57 (s, 2H), 7.39 (s, 1H), 7.33 (d, J=6.0 Hz, 1H), 7.23 (d, J=7.3 Hz, 1H), 6.96 (dd, J=32.6, 15.1 Hz, 1H), 6.85-6.72 (m, 1H), 6.61-6.57 (m, 2H), 4.50 (s, 1H), 4.43 (s, 1H), 4.09-3.82 (m, 6H), 3.77-3.69 (m, 2H), 3.63-3.55 (m, 4H), 3.31-3.26 (m, 2H), 2.93 (s, 6H), 2.84-2.78 (m, 2H), 2.46-2.41 (m, 2H), 2.26 (s, 3H), 1.71-1.67 (m, 2H), 1.29-1.27 (m, 2H). MS (ESI) [M+H]⁺=886.6.

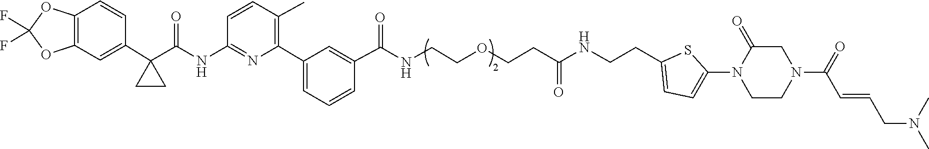

Example 332: (E)-3-(6-(1-(2,2-difluorobenzo[d][1,3]dioxol-5-yl)cyclopropane-1-carboxamido)-3-methylpyridin-2-yl)-N-(2-(2-(3-((2-(5-(4-(4-(dimethylamino)but-2-enoyl)-2-oxopiperazin-1-yl)thiophen-2-yl)ethyl)amino)-3-oxopropoxy)ethoxy)ethyl)benzamide White solid, 47% yield. ¹H NMR (400 MHz, CD₃OD) δ 8.07 (d, J=8.0 Hz, 1H), 7.93-7.78 (m, 3H), 7.58 (s, 2H), 7.39 (s, 1H), 7.33 (d, J=7.9 Hz, 1H), 7.23 (d, J=7.5 Hz, 1H), 6.96 (dd, J=35.0, 15.2 Hz, 1H), 6.84-7.72 (m, 1H), 6.69-6.62 (m, 2H), 4.51 (s, 1H), 4.44 (s, 1H), 4.10-3.85 (m, 6H), 3.71-3.56 (m, 10H), 3.40-3.33 (m, 2H), 2.96-2.85 (m, 8H), 2.42-2.34 (m, 2H), 2.26 (s, 3H), 1.71-1.67 (m, 2H), 1.29-1.27 (m, 2H). MS (ESI) [M+H]⁺=930.4.

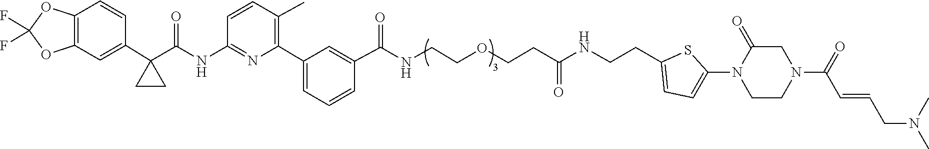

Example 333: (E)-3-(6-(1-(2,2-difluorobenzo[d][1,3]dioxol-5-yl)cyclopropane-1-carboxamido)-3-methylpyridin-2-yl)-N-(15-(5-(4-(4-(dimethylamino)but-2-enoyl)-2-oxopiperazin-1-yl)thiophen-2-yl)-12-oxo-3,6,9-trioxa-13-azapentadecyl)benzamide White solid, 45% yield. ¹H NMR (400 MHz, CD₃OD) δ 8.08 (d, J=8.0 Hz, 1H), 7.94-7.80 (m, 3H), 7.58 (s, 2H), 7.40 (s, 1H), 7.34 (d, J=8.1 Hz, 1H), 7.23 (d, J=7.9 Hz, 1H), 6.96 (dd, J=35.3, 15.1 Hz, 1H), 6.83-6.73 (m, 1H), 6.68-6.66 (m, 2H), 4.51 (s, 1H), 4.44 (s, 1H), 4.09-3.87 (m, 6H), 3.69-3.51 (m, 14H), 3.43-3.36 (m, 2H), 2.98-2.86 (m, 8H), 2.42-2.36 (d, J=7.3 Hz, 2H), 2.27 (s, 3H), 1.71-1.67 (m, 2H), 1.29-1.27 (m, 2H). MS (ESI) [M+H]⁺=974.9.

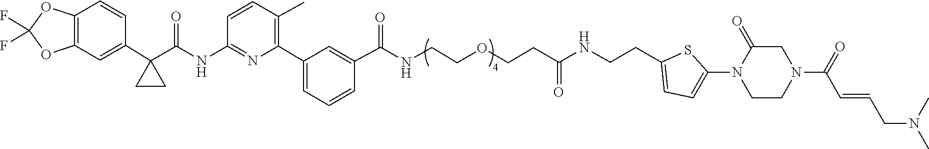

Example 334: (E)-3-(6-(1-(2,2-difluorobenzo[d][1,3]dioxol-5-yl)cyclopropane-1-carboxamido)-3-methylpyridin-2-yl)-N-(18-(5-(4-(4-(dimethylamino)but-2-enoyl)-2-oxopiperazin-1-yl)thiophen-2-yl)-15-oxo-3,6,9,12-tetraoxa-16-azaoctadecyl)benzamide White solid, 46% yield. ¹H NMR (400 MHz, CD₃OD) δ 8.08 (d, J=8.1 Hz, 1H), 7.93-7.77 (m, 3H), 7.58 (s, 2H), 7.39 (s, 1H), 7.34 (d, J=7.6 Hz, 1H), 7.23 (d, J=7.4 Hz, 1H), 6.96 (dd, J=35.4, 15.2 Hz, 1H), 6.84-6.74 (m, 1H), 6.69-6.66 (m, 2H), 4.51 (s, 1H), 4.44 (s, 1H), 4.10-3.86 (m, 6H), 3.73-3.51 (m, 18H), 3.46-3.37 (m, 2H), 2.95-2.90 (m, 8H), 2.45-2.38 (m, 2H), 2.27 (s, 3H), 1.71-1.67 (m, 2H), 1.29-1.27 (m, 2H). MS (ESI) [M+H]⁺=1018.6.

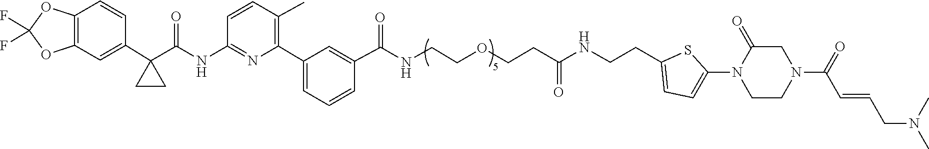

Example 335: (E)-3-(6-(1-(2,2-difluorobenzo[d][1,3]dioxol-5-yl)cyclopropane-1-carboxamido)-3-methylpyridin-2-yl)-N-(21-(5-(4-(4-(dimethylamino)but-2-enoyl)-2-oxopiperazin-1-yl)thiophen-2-yl)-18-oxo-3,6,9,12,15-pentaoxa-19-azahenicosyl)benzamide White solid, 48% yield. ¹H NMR (400 MHz, CD₃OD) δ 8.07 (d, J=7.4 Hz, 1H), 7.95-7.80 (m, 3H), 7.59 (s, 2H), 7.39 (s, 1H), 7.33 (d, J=8.1 Hz, 1H), 7.23 (d, J=7.5 Hz, 1H), 6.96 (dd, J=35.8, 15.0 Hz, 1H), 6.82-6.72 (m, 1H), 6.69-6.67 (m, 2H), 4.51 (s, 1H), 4.44 (s, 1H), 4.10-3.87 (m, 6H), 3.72-3.53 (m, 22H), 3.44-3.36 (m, 2H), 2.95-2.90 (m, 8H), 2.47-2.39 (m, 2H), 2.28 (s, 3H), 1.71-1.67 (m, 2H), 1.29-1.27 (m, 2H). MS (ESI) [M+H]⁺=1062.5.

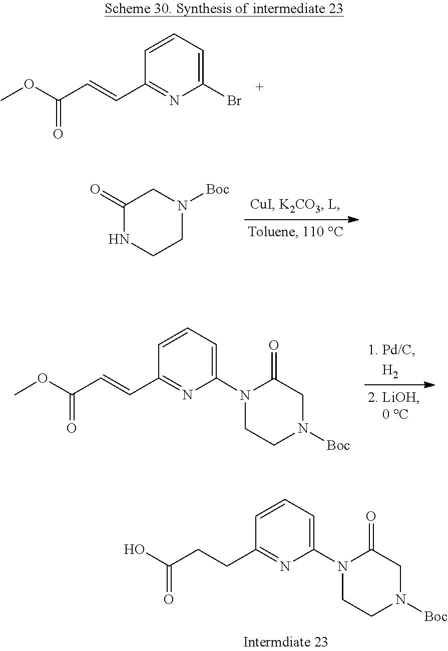 [SMILES: COC(=O)/C=C/c1cccc(Br)n1 ; CC(C)(C)OC(=O)N1CCNC(=O)C1 ; COC(=O)/C=C/c1cccc(N2CCN(C(=O)OC(C)(C)C)CC2=O)n1 ; CC(C)(C)OC(=O)N1CCN(c2cccc(CCC(=O)O)n2)C(=O)C1]

Intermediate 23: 3-(6-(4-(tert-butoxycarbonyl)-2-oxopiperazin-1-yl)pyridin-2-yl)propanoic acid Intermediate 23 was synthesized following similar procedure for preparing intermediate 13. White solid, 35% yield. ¹H NMR (400 MHz, CD₃OD) δ 7.79-7.66 (m, 2H), 7.18 (d, J=6.7 Hz, 1H), 4.26 (s, 2H), 4.10 (s, 2H), 3.78 (s, 2H), 3.17-3.08 (m, 2H), 2.83-2.73 (m, 2H), 1.53 (s, 9H). MS (ESI) [M+H]⁺=350.1.

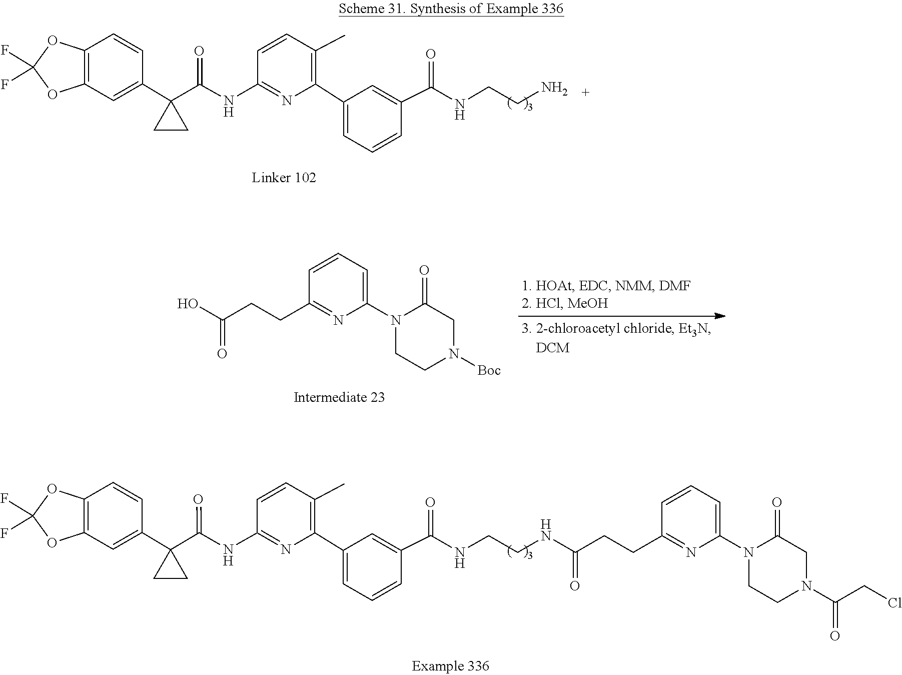

Example 336: N-(4-(3-(6-(4-(2-chloroacetyl)-2-oxopiperazin-1-yl)pyridin-2-yl)propanamido)butyl)-3-(6-(1-(2,2-difluorobenzo[d][1,3]dioxol-5-yl)cyclopropane-1-carboxamido)-3-methylpyridin-2-yl)benzamide Linker 102 (10 mg, 0.02 mmol, 1.0 eq), intermediate 23 (8.7 mg, 0.022 mmol, 1.1 eq), EDC HCl (5.7 mg, 0.03 mmol, 1.5 eq), HOAt (4.1 mg, 0.03 mmol, 1.5 eq), NMM (6.0 mg, 0.06 mmol, 3.0 eq) were stirred in DMF (1.5 mL) at room temperature for 2 h. Then the mixture was purified via Reverse-ISCO. The purified product was then dissolved in MeOH (1 mL) and HCl solution (4 M in dioxane, 1 mL, 4 mmol), stirred for 2h. Then removed all the volatiles and re-dissolved in DCM (1 mL), followed by 2-chloroacetyl chloride (2.5 mg, 0.024 mmol, 1.2 eq) and Et₃N (2.6 mg, 0.026 mmol, 1.3 eq). After stirred at rt for 5 min, removed all the volatiles. the mixture was purified via prep-HPLC to yield titled compound (2.7 mg, 16% over 3 steps). ¹H NMR (400 MHz, CD₃OD) δ 8.06 (d, J=7.5 Hz, 1H), 7.97-7.88 (m, 3H), 7.73-7.57 (m, 4H), 7.39 (s, 1H), 7.33 (d, J=6.2 Hz, 1H), 7.23 (d, J=7.9 Hz, 1H), 7.12 (d, J=7.2 Hz, 1H), 4.42 (s, 1H), 4.38-4.31 (m, 3H), 4.24-4.10 (m, 2H), 3.93 (s, 2H), 3.35 (s, 2H), 3.19 (s, 2H), 3.07 (s, 2H), 2.64 (s, 2H), 2.30 (s, 3H), 1.71 (s, 2H), 1.52 (s, 4H), 1.30 (s, 2H). MS (ESI) [M+H]⁺=830.4.

Examples 337-340 are synthesized as the same procedure for preparing example 336 from related linkers and intermediate 23.

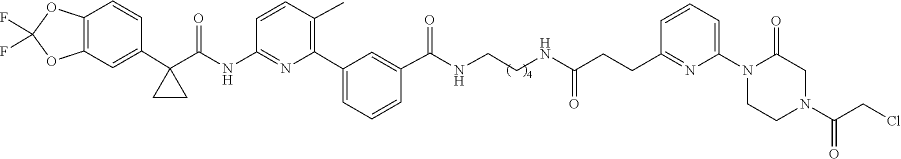

Example 337: N-(5-(3-(6-(4-(2-chloroacetyl)-2-oxopiperazin-1-yl)pyridin-2-yl)propanamido)pentyl)-3-(6-(1-(2,2-difluorobenzo[d][1,3]dioxol-5-yl)cyclopropane-1-carboxamido)-3-methylpyridin-2-yl)benzamide White solid, 15% yield. ¹H NMR (400 MHz, CD₃OD) δ 8.08 (d, J=7.4 Hz, 1H), 7.94-7.80 (m, 3H), 7.70 (s, 2H), 7.58 (s, 2H), 7.39 (s, 1H), 7.34 (d, J=5.9 Hz, 1H), 7.23 (d, J=7.2 Hz, 1H), 7.11 (s, 1H), 4.44 (s, 1H), 4.39-4.31 (m, 3H), 4.21 (s, 1H), 4.13 (s, 1H), 4.00-3.87 (m, 2H), 3.36 (s, 2H), 3.16 (s, 2H), 3.06 (s, 2H), 2.62 (s, 2H), 2.27 (s, 3H), 1.70 (s, 2H), 1.60 (s, 2H), 1.49 (s, 2H), 1.38-1.24 (m, 4H). MS (ESI) [M+H]⁺=844.9.

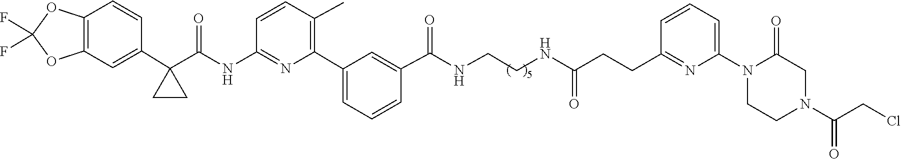

Example 338: N-(6-(3-(6-(4-(2-chloroacetyl)-2-oxopiperazin-1-yl)pyridin-2-yl)propanamido)hexyl)-3-(6-(1-(2,2-difluorobenzo[d][1,3]dioxol-5-yl)cyclopropane-1-carboxamido)-3-methylpyridin-2-yl)benzamide White solid, 14% yield. ¹H NMR (400 MHz, CD₃OD) δ 8.08 (d, J=7.6 Hz, 1H), 7.94-7.82 (m, 3H), 7.70 (s, 2H), 7.59 (s, 2H), 7.39 (s, 1H), 7.33 (d, J=7.7 Hz, 1H), 7.23 (d, J=7.6 Hz, 1H), 7.12 (s, 1H), 4.43 (s, 1H), 4.35 (s, 3H), 4.21 (s, 1H), 4.13 (s, 1H), 3.97-3.89 (m, 2H), 3.37 (s, 2H), 3.15 (s, 2H), 3.08 (s, 2H), 2.63 (s, 2H), 2.28 (s, 3H), 1.69 (s, 2H), 1.60 (s, 2H), 1.49-1.26 (m, 8H). MS (ESI) [M+H]⁺=858.5.

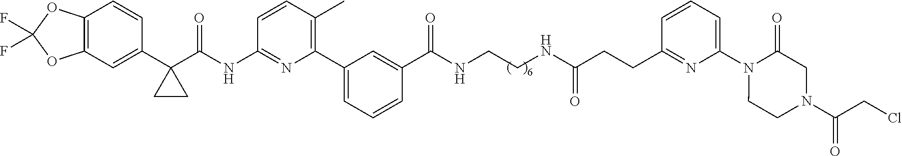

Example 339: N-(7-(3-(6-(4-(2-chloroacetyl)-2-oxopiperazin-1-yl)pyridin-2-yl)propanamido)heptyl)-3-(6-(1-(2,2-difluorobenzo[d][1,3]dioxol-5-yl)cyclopropane-1-carboxamido)-3-methylpyridin-2-yl)benzamide White solid, 12% yield. ¹H NMR (400 MHz, CD₃OD) δ 8.08 (d, J=6.8 Hz, 1H), 7.88 (s, 1H), 7.85 (s, 1H), 7.79 (d, J=7.3 Hz, 1H), 7.70 (s, 2H), 7.57 (s, 2H), 7.39 (s, 1H), 7.34 (d, J=7.9 Hz, 1H), 7.23 (d, J=7.3 Hz, 1H), 7.12 (s, 1H), 4.44 (s, 1H), 4.36 (s, 3H), 4.20 (s, 1H), 4.13 (s, 1H), 3.99-3.87 (m, 2H), 3.40-3.35 (d, J=7.6 Hz, 2H), 3.15 (s, 2H), 3.08 (s, 2H), 2.63 (s, 2H), 2.27 (s, 3H), 1.69 (s, 2H), 1.61 (s, 2H), 1.45 (s, 2H), 1.41-1.23 (m, 8H). MS (ESI) [M+H]⁺=872.5.

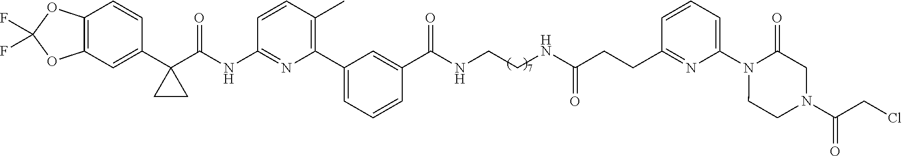

Example 340: N-(8-(3-(6-(4-(2-chloroacetyl)-2-oxopiperazin-1-yl)pyridin-2-yl)propanamido)octyl)-3-(6-(1-(2,2-difluorobenzo[d][1,3]dioxol-5-yl)cyclopropane-1-carboxamido)-3-methylpyridin-2-yl)benzamide White solid, 16% yield. ¹H NMR (400 MHz, CD₃OD) δ 8.08 (d, J=7.9 Hz, 1H), 7.95-7.84 (m, 3H), 7.72 (s, 2H), 7.60 (s, 2H), 7.39 (s, 1H), 7.33 (d, J=6.8 Hz, 1H), 7.23 (d, J=8.4 Hz, 1H), 7.13 (s, 1H), 4.44 (s, 1H), 4.36 (s, 3H), 4.21 (s, 1H), 4.13 (s, 1H), 3.98-3.88 (m, 2H), 3.41-3.35 (m, 2H), 3.17-3.04 (m, 4H), 2.62 (s, 2H), 2.29 (s, 3H), 1.70 (s, 2H), 1.62 (s, 2H), 1.48-1.21 (m, 12H). MS (ESI) [M+H]⁺=886.5.

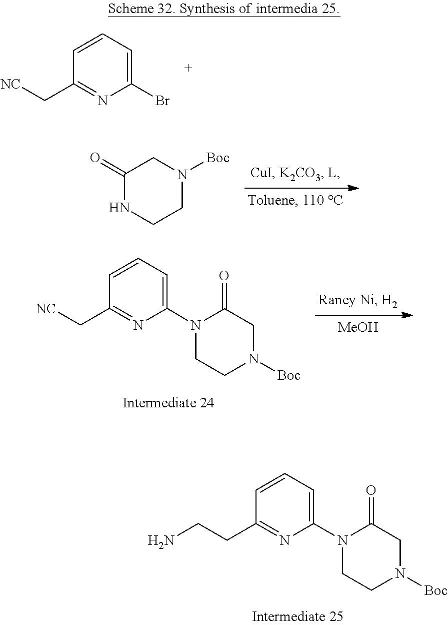

Intermediate 25: tert-butyl 4-(6-(2-aminoethyl)pyridin-2-yl)-3-oxopiperazine-1-carboxylate 2-(6-bromopyridin-2-yl)acetonitrile (196 mg, 1.0 mmol, 1.0 eq), tert-butyl 3-oxopiperazine-1-carboxylate (300 mg, 1.5 mmol, 1.5 eq), CuI (85 mg, 0.5 mmol), N¹,N²-dimethylethane-1,2-diamine (88 mg, 1.0 mmol, 1.0 eq), K₂CO₃ (276 mg, 2.0 mmol, 2.0 eq) were added Toluene (5 mL). The mixture was stirred at 110° C. under N₂ for 6 h. The mixture was purified by ISCO to yield intermediate 17 as a white solid (205 mg, 65%).¹H NMR (400 MHz, CD₃OD) δ 7.96-7.83 (m, 2H), 7.31 (d, J=7.9 Hz, 1H), 4.29 (s, 2H), 4.18 (s, 2H), 4.10 (s, 2H), 3.80 (s, 2H), 1.55 (s, 9H). MS (ESI) [M+H]⁺=317.4.

Intermediate 24 (160 mg, 0.5 mmol, 1.0 eq), Raney Ni (30 mg) were stirred in MeOH (2 mL) at room temperature under 1 atm H₂ for 30 min. Then the mixture was purified by reverse-ISCO to yield titled compound as a white solid (120 mg, 75%). ¹H NMR (400 MHz, CD₃OD) δ 7.69-7.62 (m, 1H), 7.49 (d, J=8.8 Hz, 1H), 7.32 (d, J=9.9 Hz, 1H), 3.38-3.31 (m, 2H), 3.15-3.08 (m, 2H). MS (ESI) [M+H]⁺=321.5.

Example 341-345 were synthesized following similar procedure for preparing example 336 from related linker and intermediate 25.

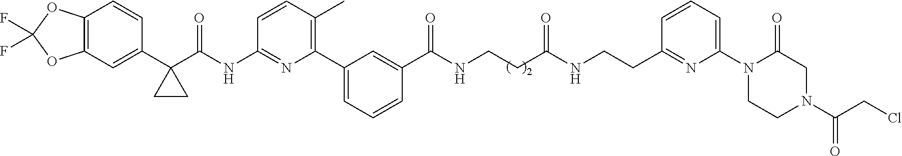

Example 341: N-(4-((2-(6-(4-(2-chloroacetyl)-2-oxopiperazin-1-yl)pyridin-2-yl)ethyl)amino)-4-oxobutyl)-3-(6-(1-(2,2-difluorobenzo[d][1,3]dioxol-5-yl)cyclopropane-1-carboxamido)-3-methylpyridin-2-yl)benzamide White solid, 18% yield. ¹H NMR (400 MHz, CD₃OD) δ 8.06 (d, J=7.8 Hz, 1H), 7.95-7.86 (m, 3H), 7.77-7.56 (m, 4H), 7.39 (s, 1H), 7.33 (d, J=7.3 Hz, 1H), 7.23 (d, J=7.2 Hz, 1H), 7.12 (d, J=7.2 Hz, 1H), 4.43 (s, 1H), 4.34 (s, 3H), 4.21 (s, 1H), 4.12 (s, 1H), 3.99-3.85 (m, 2H), 3.59 (s, 2H), 3.38-3.33 (m, 2H), 2.95 (s, 2H), 2.29 (s, 3H), 2.22 (s, 2H), 1.84 (s, 2H), 1.71 (s, 2H), 1.29 (s, 2H). MS (ESI) [M+H]⁺=816.4.

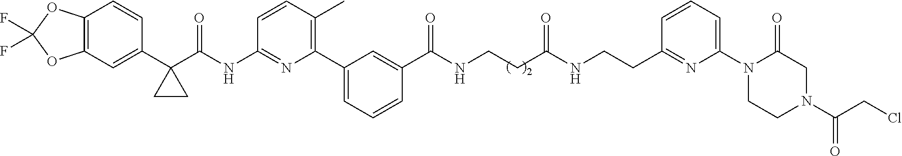

Example 342: N-(5-((2-(6-(4-(2-chloroacetyl)-2-oxopiperazin-1-yl)pyridin-2-yl)ethyl)amino)-5-oxopentyl)-3-(6-(1-(2,2-difluorobenzo[d][1,3]dioxol-5-yl)cyclopropane-1-carboxamido)-3-methylpyridin-2-yl)benzamide White solid, 15% yield. ¹H NMR (400 MHz, CD₃OD) δ 8.08 (d, J=7.7 Hz, 1H), 7.95-7.83 (m, 3H), 7.74-7.55 (m, 4H), 7.39 (s, 1H), 7.33 (d, J=7.0 Hz, 1H), 7.23 (d, J=7.0 Hz, 1H), 7.12 (d, J=5.9 Hz, 1H), 4.44 (s, 1H), 4.36 (s, 3H), 4.21 (s, 1H), 4.13 (s, 1H), 3.98-3.88 (m, 2H), 3.61 (s, 2H), 3.38-3.34 (m, 2H), 2.96 (s, 2H), 2.28 (s, 3H), 2.19 (s, 2H), 1.70 (s, 2H), 1.62 (s, 2H), 1.54 (s, 2H), 1.27 (s, 2H). MS (ESI) [M+H]⁺=830.4.

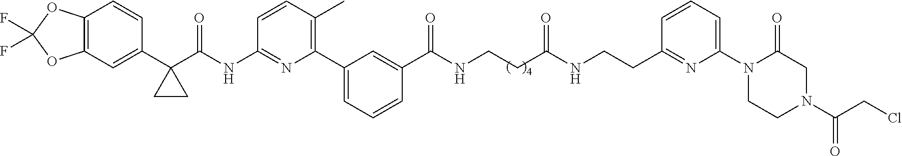

Example 343: N-(6-((2-(6-(4-(2-chloroacetyl)-2-oxopiperazin-1-yl)pyridin-2-yl)ethyl)amino)-6-oxohexyl)-3-(6-(1-(2,2-difluorobenzo[d][1,3]dioxol-5-yl)cyclopropane-1-carboxamido)-3-methylpyridin-2-yl)benzamide White solid, 17% yield. ¹H NMR (400 MHz, CD₃OD) δ 8.07 (d, J=7.2 Hz, 1H), 7.94-7.82 (m, 3H), 7.78-7.66 (m, 2H), 7.60 (s, 2H), 7.40 (s, 1H), 7.34 (d, J=7.5 Hz, 1H), 7.24 (d, J=6.7 Hz, 1H), 7.11 (d, J=7.5 Hz, 1H), 4.45 (s, 1H), 4.37 (s, 3H), 4.22 (s, 1H), 4.13 (s, 1H), 3.98-3.88 (m, 2H), 3.58 (s, 2H), 3.39-3.34 (m, 2H), 2.94 (s, 2H), 2.29 (s, 3H), 2.16 (s, 2H), 1.71 (s, 2H), 1.60 (s, 4H), 1.39-1.26 (m 4H). MS (ESI) [M+H]⁺=844.5.

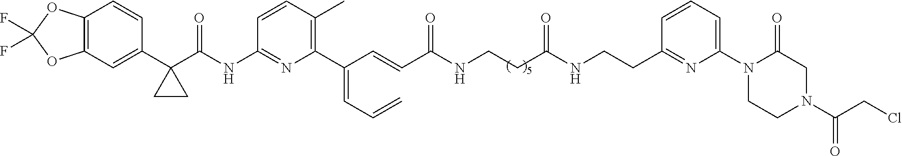

Example 344: N-(7-((2-(6-(4-(2-chloroacetyl)-2-oxopiperazin-1-yl)pyridin-2-yl)ethyl)amino)-7-oxoheptyl)-3-(6-(1-(2,2-difluorobenzo[d][1,3]dioxol-5-yl)cyclopropane-1-carboxamido)-3-methylpyridin-2-yl)benzamide White solid, 14% yield. ¹H NMR (400 MHz, CD₃OD) δ 8.08 (d, J=8.3 Hz, 1H), 7.94-7.81 (m, 3H), 7.76-7.66 (m, 2H), 7.59 (s, 2H), 7.39 (s, 1H), 7.34 (d, J=8.1 Hz, 1H), 7.23 (d, J=6.1 Hz, 1H), 7.13 (s, 1H), 4.44 (s, 1H), 4.36 (s, 3H), 4.23 (s, 1H), 4.14 (s, 1H), 3.98-3.88 (m, 2H), 3.60 (s, 2H), 3.39-3.33 (m, 2H), 2.97 (s, 2H), 2.28 (s, 3H), 2.14 (s, 2H), 1.70 (s, 2H), 1.57 (br, 4H), 1.41-1.22 (m, 6H). MS (ESI) [M+H]⁺=858.5.

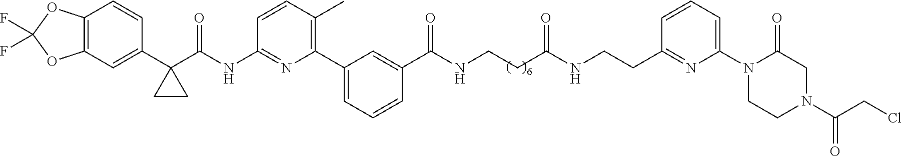

Example 345: N-(8-((2-(6-(4-(2-chloroacetyl)-2-oxopiperazin-1-yl)pyridin-2-yl)ethyl)amino)-8-oxooctyl)-3-(6-(1-(2,2-difluorobenzo[d][1,3]dioxol-5-yl)cyclopropane-1-carboxamido)-3-methylpyridin-2-yl)benzamide White solid, 13% yield. ¹H NMR (400 MHz, CD₃OD) δ 8.08 (d, J=7.9 Hz, 1H), 7.92-7.68 (m, 5H), 7.57 (s, 2H), 7.39 (s, 1H), 7.34 (d, J=7.8 Hz, 1H), 7.23 (d, J=6.9 Hz, 1H), 7.12 (s, 1H), 4.44 (s, 1H), 4.37 (s, 3H), 4.23 (s, 1H), 4.14 (s, 1H), 3.99-3.89 (m, 2H), 3.60 (s, 2H), 3.41-3.34 (m, 2H), 2.96 (s, 2H), 2.27 (s, 3H), 2.13 (s, 2H), 1.69 (s, 2H), 1.64-1.52 (dm, 4H), 1.40-1.22 (m, 8H). MS (ESI) [M+H]⁺=872.5.

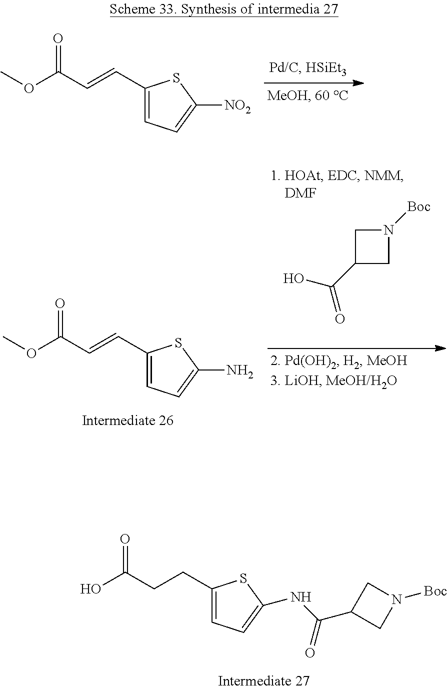 [SMILES: COC(=O)/C=C/c1ccc([N+](=O)[O-])s1 ; COC(=O)/C=C/c1ccc(N)s1 ; CC(C)(C)OC(=O)N1CC(C(=O)O)C1 ; CC(C)(C)OC(=O)N1CC(C(=O)Nc2ccc(CCC(=O)O)s2)C1]

Intermediate 27: 3-(5-(1-(tert-butoxycarbonyl)azetidine-3-carboxamido)thiophen-2-yl)propanoic acid Methyl (E)-3-(5-nitrothiophen-2-yl)acrylate (416 mg, 2.0 mmol, 1.0 eq), Pd/C (120 mg), HSiEt₃ (1.16 g, 10 mmol, 5.0 eq) and 2.5 mL MeOH were stirred in a sealed tube at 60° C. for 30 min. Then the mixture was filtered and purified with silica gel column (Hexane/Ethyl acetate=1:1) to afford intermediate 26 as yellow solid (200 mg, 49%). ¹H NMR (400 MHz, Methanol-d4) δ 7.64 (s, 1H), 6.96 (s, 1H), 5.99 (s, 1H), 5.65 (s, 1H), 3.73 (s, 3H). LCMS m/z=184.1.

Intermediate 26 (180 mg, 1.0 mmol, 1.0 eq), 1-(tert-butoxycarbonyl)azetidine-3-carboxylic acid (201 mg, 1.0 mmol, 1.0 eq), EDC-HCl (288 mg, 1.5 mmol, 1.5 eq), HOAt (204 mg, 1.5 mmol, 1.5 eq), NMM (300 mg, 3.0 mmol, 3.0 eq) were stirred in DMF (2.5 mL) at room temperature for 2 h. Then the mixture was purified via RP-C18 [MeCN/Water (0.10% TFA)] to give 140 mg yellow solid without further purification. MS (ESI) [M+H]⁺=367.2.

The crude product and Pd(OH)₂ (70 mg) were stirred in MeOH (5 mL) at 60° C. under 1 atm H₂ overnight. Then the mixture was filtered. To the filtrate, LiOH (56 mg, 2.3 mmol, 3.0 eq) and H₂O (2 mL) were added, the mixture was stirred at room temperature for 1 h followed by purified via RP-C18 [MeCN/Water (0.1% TFA)] to afford colorless solid (65 mg, 18% over 3 steps). ¹H NMR (400 MHz, Methanol-d₄) δ 6.60 (d, J=3.8 Hz, 1H), 6.54 (d, J=3.8 Hz, 1H), 4.15 (s, 2H), 4.05 (s, 2H), 3.24-3.20 (m, 1H), 3.04 (t, J=7.5 Hz, 2H), 2.56 (t, J=7.4 Hz, 2H), 1.46 (s, 9H). MS (ESI) [M+H]⁺=355.2.

Examples 346-351 were synthesized following the same procedure for preparing example 336 from related linkers and intermediate 27.

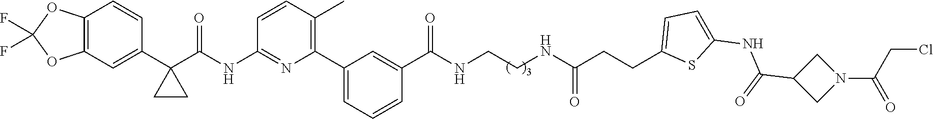

Example 346: 1-(2-chloroacetyl)-N-(5-(3-((4-(3-(6-(1-(2,2-difluorobenzo[d][1,3]dioxol-5-yl)cyclopropane-1-carboxamido)-3-methylpyridin-2-yl)benzamido)butyl)amino)-3-oxopropyl)thiophen-2-yl)azetidine-3-carboxamide White solid, 35% yield. ¹H NMR (400 MHz, Methanol-d₄) δ 8.08 (d, J=8.5 Hz, 1H), 7.93-7.89 (m, 1H), 7.86 (s, 1H), 7.81 (d, J=8.5 Hz, 1H), 7.60-7.57 (d, J=5.9 Hz, 2H), 7.39 (s, 1H), 7.34 (d, J=7.9 Hz, 1H), 7.23 (d, J=8.2 Hz, 1H), 6.58 (d, J=3.7 Hz, 1H), 6.52 (d, J=3.8 Hz, 1H), 4.48-4.42 (m, 2H), 4.24-4.10 (m, 2H), 4.04 (s, 2H), 3.60-3.54 (m, 1H), 3.38-3.35 (m, 2H), 3.24-3.19 (m, 2H), 3.05 (t, J=7.2 Hz, 2H), 2.50 (t, J=7.2 Hz, 2H), 2.28 (s, 3H), 1.70 (q, J=4.0 Hz, 2H), 1.56 (s, 4H), 1.27 (q, J=4.0 Hz, 2H). MS (ESI) [M+H]⁺=835.3.

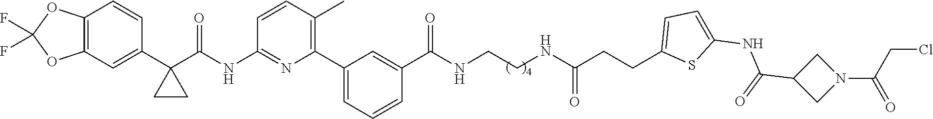

Example 347: 1-(2-chloroacetyl)-N-(5-(3-((5-(3-(6-(1-(2,2-difluorobenzo[d][1,3]dioxol-5-yl)cyclopropane-1-carboxamido)-3-methylpyridin-2-yl)benzamido)pentyl)amino)-3-oxopropyl)thiophen-2-yl)azetidine-3-carboxamide White solid, 36% yield. ¹H NMR (400 MHz, Methanol-d₄) δ 8.11-8.06 (m, 1H), 7.97-7.89 (m, 3H), 7.66-7.59 (m, 2H), 7.41 (s, 1H), 7.39-7.33 (m, 1H), 7.27-7.23 (m, 1H), 6.57 (s, 1H), 6.56-6.51 (m, 1H), 4.49-4.43 (m, 2H), 4.25-4.11 (m, 2H), 4.05 (s, 2H), 3.60 (s, 1H), 3.40-3.36 (m, 2H), 3.23-3.14 (m, 2H), 3.07-3.00 (m, 2H), 2.52-2.44 (m, 2H), 2.31 (s, 3H), 1.73 (s, 2H), 1.62 (s, 2H), 1.52 (s, 2H), 1.32 (s, 4H). MS (ESI) [M+H]⁺=849.3.

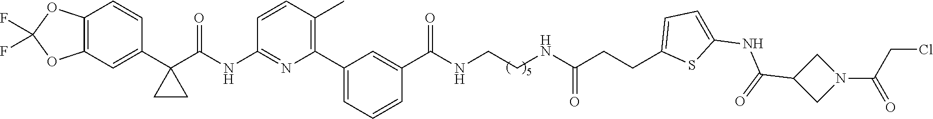

Example 348: 1-(2-chloroacetyl)-N-(5-(3-((6-(3-(6-(1-(2,2-difluorobenzo[d][1,3]dioxol-5-yl)cyclopropane-1-carboxamido)-3-methylpyridin-2-yl)benzamido)hexyl)amino)-3-oxopropyl)thiophen-2-yl)azetidine-3-carboxamide White solid, 32% yield. ¹H NMR (400 MHz, Methanol-d₄) δ 8.09 (d, J=8.5 Hz, 1H), 7.94-7.90 (m, 1H), 7.88 (s, 1H), 7.84 (d, J=8.5 Hz, 1H), 7.62-7.58 (m, 2H), 7.40 (d, J=1.8 Hz, 1H), 7.36-7.32 (m, 1H), 7.24 (d, J=8.2 Hz, 1H), 6.58 (d, J=3.8 Hz, 1H), 6.52 (d, J=3.8 Hz, 1H), 4.50-4.43 (m, 2H), 4.26-4.13 (m, 2H), 4.05 (s, 2H), 3.64-3.56 (m, 1H), 3.38 (t, J=7.2 Hz, 2H), 3.16 (t, J=6.8 Hz, 2H), 3.04 (t, J=7.2 Hz, 2H), 2.50 (t, J=7.3 Hz, 2H), 2.29 (s, 3H), 1.70 (q, J=4.0 Hz, 2H), 1.64-1.56 (m, 2H), 1.52-1.44 (m, 2H), 1.40-1.25 (m, 6H MS (ESI) [M+H]⁺=863.3.

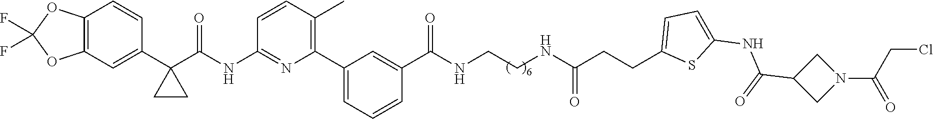

Example 349: 1-(2-chloroacetyl)-N-(5-(3-((7-(3-(6-(1-(2,2-difluorobenzo[d][1,3]dioxol-5-yl)cyclopropane-1-carboxamido)-3-methylpyridin-2-yl)benzamido)heptyl)amino)-3-oxopropyl)thiophen-2-yl)azetidine-3-carboxamide White solid, 25% yield. ¹H NMR (400 MHz, Methanol-d₄) δ 8.09 (d, J=8.5 Hz, 1H), 7.94-7.88 (m, 1H), 7.87 (s, 1H), 7.83 (d, J=8.5 Hz, 1H), 7.61-7.55 (m, 2H), 7.40 (d, J=1.7 Hz, 1H), 7.34 (dd, J=8.2, 1.8 Hz, 1H), 7.24 (d, J=8.2 Hz, 1H), 6.57 (d, J=3.8 Hz, 1H), 6.52 (d, J=3.8 Hz, 1H), 4.53-4.43 (m, 2H), 4.27-4.13 (m, 2H), 4.05 (s, 2H), 3.65-3.57 (m, 1H), 3.38 (t, J=7.2 Hz, 2H), 3.15 (t, J=6.9 Hz, 2H), 3.04 (t, J=7.3 Hz, 2H), 2.50 (t, J=7.3 Hz, 2H), 2.28 (s, 3H), 1.70 (q, J=4.0 Hz, 2H), 1.66-1.58 (m, 2H), 1.50-1.42 (m, 2H), 1.41-1.24 (m, 8H). MS (ESI) [M+H]⁺=877.3.

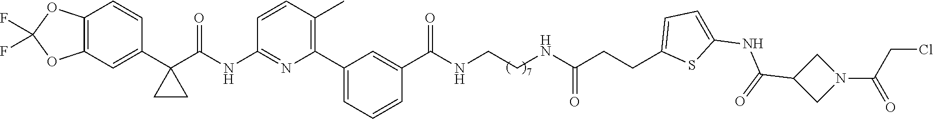

Example 350: 1-(2-chloroacetyl)-N-(5-(3-((8-(3-(6-(1-(2,2-difluorobenzo[d][1,3]dioxol-5-yl)cyclopropane-1-carboxamido)-3-methylpyridin-2-yl)benzamido)octyl)amino)-3-oxopropyl)thiophen-2-yl)azetidine-3-carboxamide White solid, 26% yield. ¹H NMR (400 MHz, Methanol-d₄) δ 8.09 (d, J=8.5 Hz, 1H), 7.93-7.88 (m, 1H), 7.86 (s, 1H), 7.81 (d, J=8.6 Hz, 1H), 7.61-7.55 (m, 2H), 7.40 (d, J=1.8 Hz, 1H), 7.34 (dd, J=8.3, 1.8 Hz, 1H), 7.24 (d, J=8.3 Hz, 1H), 6.58 (d, J=3.8 Hz, 1H), 6.53 (d, J=3.8 Hz, 1H), 4.52-4.44 (m, 2H), 4.26-4.14 (m, 2H), 4.05 (s, 2H), 3.65-3.55 (m, 1H), 3.39 (t, J=7.3 Hz, 2H), 3.14 (t, J=6.9 Hz, 2H), 3.04 (t, J=7.2 Hz, 2H), 2.50 (t, J=7.3 Hz, 2H), 2.28 (s, 3H), 1.70 (q, J=4.0 Hz, 2H), 1.66-1.56 (m, 2H), 1.48-1.23 (m, 12H). MS (ESI) [M+H]⁺=891.3.

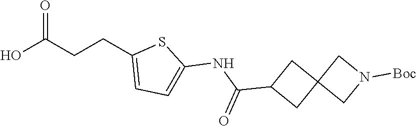 [SMILES: CC(C)(C)OC(=O)N1CC2(CC(C(=O)Nc3ccc(CCC(=O)O)s3)C2)C1]

### Intermediate 28

Intermediate 28: 3-(5-(2-(tert-butoxycarbonyl)-2-azaspiro[3.3]heptane-6-carboxamido)thiophen-2-yl)propanoic acid Intermediate 28 was synthesized following the same procedure for preparing intermediate 27. Yellow solid, 20% yield. ¹H NMR (400 MHz, Methanol-d₄) δ 6.59 (d, J=3.8 Hz, 1H), 6.49 (d, J=3.8 Hz, 1H), 3.98 (s, 2H), 3.89 (s, 2H), 3.12-3.08 (m, 1H), 3.03 (t, J=7.5 Hz, 2H), 2.63 (t, J=7.4 Hz, 2H), 2.49-2.42 (m, 4H), 1.45 (s, 9H). MS (ESI) [M+H]⁺=395.2.

Examples 351-355 were synthesized following the same procedure for preparing example 336 from related linkers and intermediate 28.

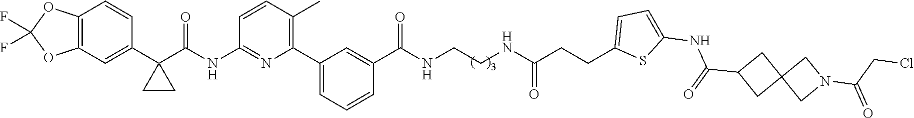

### Example 351: 2-(2-chloroacetyl)-N-(5-(3-((4-(3-(6-(1-(2,2-difluorobenzo[d][1,3]dioxol-5-yl)cyclopropane-1-carboxamido)-3-methylpyridin-2-yl)benzamido)butyl)amino)-3-oxopropyl)thiophen-2-yl)-2-azaspiro[3.3]heptane-6-carboxamide

White solid, 31% yield. ¹H NMR (400 MHz, Methanol-d₄) δ 8.09 (d, J=8.5 Hz, 1H), 7.92-7.88 (m, 1H), 7.85 (s, 1H), 7.77 (d, J=8.5 Hz, 1H), 7.60-7.53 (m, 2H), 7.39 (d, J=1.8 Hz, 1H), 7.36-7.29 (m, 1H), 7.23 (d, J=8.3 Hz, 1H), 6.56 (s, 1H), 6.47 (d, J=3.8 Hz, 1H), 4.33 (s, 1H), 4.23 (s, 1H), 4.05 (s, 1H), 4.02 (s, 1H), 4.00 (s, 1H), 3.97 (s, 1H), 3.40-3.36 (m, 2H), 3.24-3.18 (m, 2H), 3.16-3.12 (m, 1H), 3.04 (t, J=7.7 Hz, 2H), 2.52-2.42 (m, 6H), 2.26 (s, 3H), 1.69 (q, J=3.9 Hz, 2H), 1.56 (s, 4H), 1.26 (q, J=4.0 Hz, 2H). MS (ESI) [M+H]⁺=875.3.

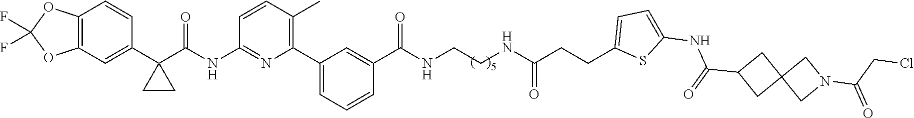

### Example 352: 2-(2-chloroacetyl)-N-(5-(3-((5-(3-(6-(1-(2,2-difluorobenzo[d][1,3]dioxol-5-yl)cyclopropane-1-carboxamido)-3-methylpyridin-2-yl)benzamido)pentyl)amino)-3-oxopropyl)thiophen-2-yl)-2-azaspiro[3.3]heptane-6-carboxamide

White solid, 33% yield. ¹H NMR (400 MHz, Methanol-d₄) δ 8.09 (d, J=8.5 Hz, 1H), 7.94-7.89 (m, 1H), 7.87 (s, 1H), 7.82 (d, J=8.3 Hz, 1H), 7.61-7.55 (m, 2H), 7.40 (d, J=1.7 Hz, 1H), 7.34 (dd, J=8.2, 1.8 Hz, 1H), 7.24 (d, J=8.3 Hz, 1H), 6.57-6.53 (m, 1H), 6.48 (d, J=3.7 Hz, 1H), 4.30 (s, 1H), 4.24 (s, 1H), 4.04 (s, 1H), 4.00 (s, 2H), 3.98 (s, 1H), 3.40-3.35 (m, 2H), 3.18 (t, J=6.7 Hz, 2H), 3.13-3.07 (m, 1H), 3.02 (t, J=7.2 Hz, 2H), 2.51-2.44 (m, 6H), 2.28 (s, 3H), 1.70 (q, J=4.0 Hz, 2H), 1.60 (p, J=7.4 Hz, 2H), 1.51 (p, J=7.0 Hz, 2H), 1.37-1.25 (m, 4H). MS (ESI) [M+H]⁺=889.3.

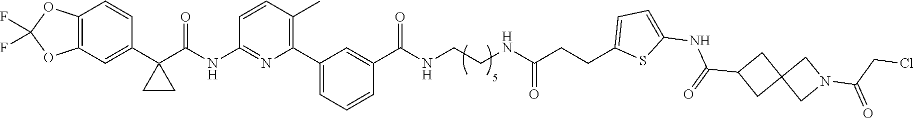

### Example 353: 2-(2-chloroacetyl)-N-(5-(3-((6-(3-(6-(1-(2,2-difluorobenzo[d][1,3]dioxol-5-yl)cyclopropane-1-carboxamido)-3-methylpyridin-2-yl)benzamido)hexyl)amino)-3-oxopropyl)thiophen-2-yl)-2-azaspiro[3.3]heptane-6-carboxamide

White solid, 35% yield. ¹H NMR (400 MHz, Methanol-d₄) δ 8.09 (d, J=8.5 Hz, 1H), 7.94-7.89 (m, 1H), 7.87 (s, 1H), 7.81 (d, J=8.6 Hz, 1H), 7.62-7.53 (m, 2H), 7.40 (d, J=1.7 Hz, 1H), 7.34 (dd, J=8.2, 1.8 Hz, 1H), 7.24 (d, J=8.3 Hz, 1H), 6.56 (d, J=3.8 Hz, 1H), 6.48 (d, J=3.8 Hz, 1H), 4.31 (s, 1H), 4.25 (s, 1H), 4.05 (s, 1H), 4.01 (s, 2H), 3.99 (s, 1H), 3.41-3.36 (m, 2H), 3.19-2.99 (m, 5H), 2.54-2.46 (m, 6H), 2.28 (s, 3H), 1.70 (q, J=4.0 Hz, 2H), 1.60 (p, J=7.1 Hz, 2H), 1.47 (p, J=6.9 Hz, 2H), 1.39-1.26 (m, 6H). MS (ESI) [M+H]⁺=903.3.

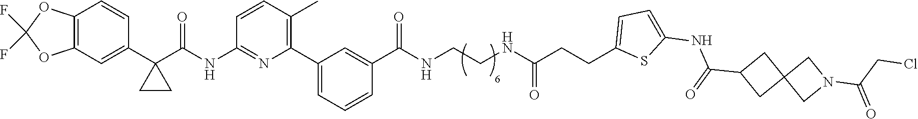

### Example 354: 2-(2-chloroacetyl)-N-(5-(3-((7-(3-(6-(1-(2,2-difluorobenzo[d][1,3]dioxol-5-yl)cyclopropane-1-carboxamido)-3-methylpyridin-2-yl)benzamido)heptyl)amino)-3-oxopropyl)thiophen-2-yl)-2-azaspiro[3.3]heptane-6-carboxamide

White solid, 32% yield. ¹H NMR (400 MHz, Methanol-d₄) δ 8.09 (d, J=8.5 Hz, 1H), 7.93-7.88 (m, 1H), 7.86 (s, 1H), 7.80 (d, J=8.5 Hz, 1H), 7.60-7.55 (m, 2H), 7.40 (d, J=1.7 Hz, 1H), 7.34 (dd, J=8.3, 1.8 Hz, 1H), 7.24 (d, J=8.3 Hz, 1H), 6.55 (d, J=3.7 Hz, 1H), 6.47 (d, J=3.7 Hz, 1H), 4.32 (s, 1H), 4.26 (s, 1H), 4.06 (s, 1H), 4.01 (s, 3H), 3.38 (t, J=7.0 Hz, 2H), 3.18-3.00 (m, 5H), 2.52-2.46 (m, 6H), 2.27 (s, 3H), 1.70 (q, J=4.0 Hz, 2H), 1.61 (p, J=6.7 Hz, 2H), 1.50-1.27 (m, 10H). MS (ESI) [M+H]⁻=917.3.

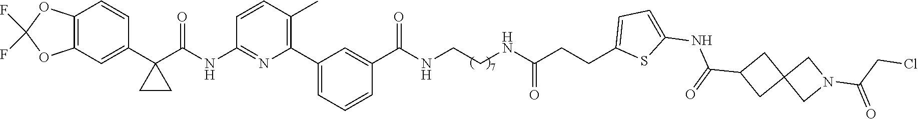

### Example 355: 2-(2-chloroacetyl)-N-(5-(3-((8-(3-(6-(1-(2,2-difluorobenzo[d][1,3]dioxol-5-yl)cyclopropane-1-carboxamido)-3-methylpyridin-2-yl)benzamido)octyl)amino)-3-oxopropyl)thiophen-2-yl)-2-azaspiro[3.3]heptane-6-carboxamide

White solid, 31% yield. ¹H NMR (400 MHz, Methanol-d4) δ 8.10 (d, J=8.5 Hz, 1H), 7.91-7.87 (m, 1H), 7.85 (s, 1H), 7.78 (d, J=8.5 Hz, 1H), 7.59-7.55 (m, 2H), 7.40 (d, J=1.7 Hz, 1H), 7.36-7.32 (m, 1H), 7.24 (d, J=8.3 Hz, 1H), 6.55 (s, 1H), 6.48 (d, J=3.7 Hz, 1H), 4.32 (s, 1H), 4.27 (s, 1H), 4.06 (s, 1H), 4.01 (d, 0.1=3.6 Hz, 3H), 3.39 (t, 0.1=7.2 Hz, 2H), 3.18-3.00 (m, 5H), 2.55-2.40 (m, 6H), 2.27 (s, 3H), 1.70 (q, J=4.0 Hz, 2H), 1.62 (p, J=7.2 Hz, 2H), 1.49-1.26 (m, 12H). MS (ESI) [M+H]⁺=931.3.

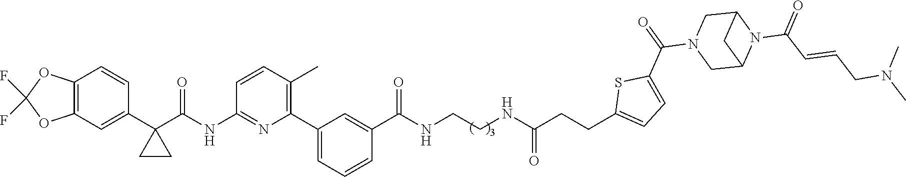

Example 356: (E)-3-(6-(1-(2,2-difluorobenzo[d][1,3]dioxol-5-yl)cyclopropane-1-carboxamido)-3-methylpyridin-2-yl)-N-(4-(3-(5-(6-(4-(dimethylamino)but-2-enoyl)-3,6-diazabicyclo[3.1.1]heptane-3-carbonyl)thiophen-2-yl)propanamido)butyl)benzamide (QC192-153) White solid, 31% yield. ¹H NMR (400 MHz, Methanol-d₄) δ 8.00 (d, J=8.5 Hz, 1H), 7.87-7.77 (m, 3H), 7.56-7.46 (m, 2H), 7.38-7.30 (m, 2H), 7.25 (dd, J=8.2, 1.7 Hz, 1H), 7.15 (d, J=8.2 Hz, 1H), 6.81 (d, J=3.8 Hz, 1H), 6.73-6.64 (m, 1H), 6.50 (d, J=15.2 Hz, 1H), 4.70 (s, 1H), 4.46 (s, 1H), 4.35-3.73 (m, 6H), 3.32-3.26 (m, 2H), 3.12 (t, J=6.2 Hz, 2H), 3.06 (t, J=7.2 Hz, 2H), 2.85-2.69 (m, 7H), 2.47 (t, J=7.2 Hz, 2H), 2.21 (s, 3H), 1.65-1.59 (m, 3H), 1.54-1.40 (m, 4H), 1.20 (q, J=4.0 Hz, 2H). MS (ESI) [M+H]⁺=896.4.

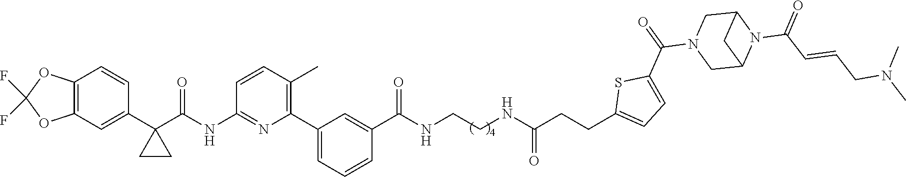

Example 357: (E)-3-(6-(1-(2,2-difluorobenzo[d][1,3]dioxol-5-yl)cyclopropane-1-carboxamido)-3-methylpyridin-2-yl)-N-(5-(3-(5-(6-(4-(dimethylamino)but-2-enoyl)-3,6-diazabicyclo[3.1.1]heptane-3-carbonyl)thiophen-2-yl)propanamido)pentyl)benzamide (QC192-154) White solid, 28% yield. ¹H NMR (400 MHz, Methanol-d₄) δ 7.99 (d, J=8.6 Hz, 1H), 7.89-7.77 (m, 3H), 7.60-7.47 (m, 2H), 7.40-7.29 (m, 2H), 7.25 (dd, J=8.3, 1.7 Hz, 1H), 7.15 (d, J=8.3 Hz, 1H), 6.79 (d, J=3.8 Hz, 1H), 6.73-6.63 (m, 1H), 6.49 (d, J=15.1 Hz, 1H), 4.70 (s, 1H), 4.47 (s, 1H), 4.37-3.76 (m, 6H), 3.32-3.26 (m, 2H), 3.12-3.00 (m, 4H), 2.86-2.67 (m, 7H), 2.45 (t, J=7.2 Hz, 2H), 2.21 (s, 3H), 1.66-1.58 (m, 3H), 1.58-1.38 (m, 4H), 1.32-1.18 (m, 4H). MS (ESI) [M+H]⁺=910.4.

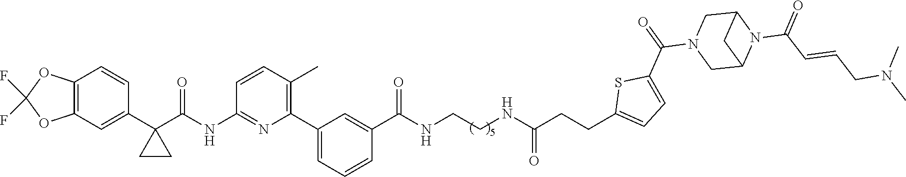

Example 358: (E)-3-(6-(1-(2,2-difluorobenzo[d][1,3]dioxol-5-yl)cyclopropane-1-carboxamido)-3-methylpyridin-2-yl)-N-(6-(3-(5-(6-(4-(dimethylamino)but-2-enoyl)-3,6-diazabicyclo[3.1.1]heptane-3-carbonyl)thiophen-2-yl)propanamido)hexyl)benzamide (QC192-155) White solid, 30% yield. ¹H NMR (400 MHz, Methanol-d₄) δ 8.00 (d, J=8.6 Hz, 1H), 7.87-7.78 (m, 3H), 7.56-7.48 (m, 2H), 7.41-7.30 (m, 2H), 7.25 (dd, J=8.3, 1.7 Hz, 1H), 7.15 (d, J=8.3 Hz, 1H), 6.81 (d, J=3.8 Hz, 1H), 6.73-6.64 (m, 1H), 6.50 (d, J=15.0 Hz, 1H), 4.70 (s, 1H), 4.47 (s, 1H), 4.37-3.77 (m, 6H), 3.34-3.27 (m, 2H), 3.11-3.02 (m, 4H), 2.87-2.68 (m, 7H), 2.46 (t, J=7.2 Hz, 2H), 2.21 (s, 3H), 1.66-1.18 (m, 13H). MS (ESI) [M+H]⁺=924.4.

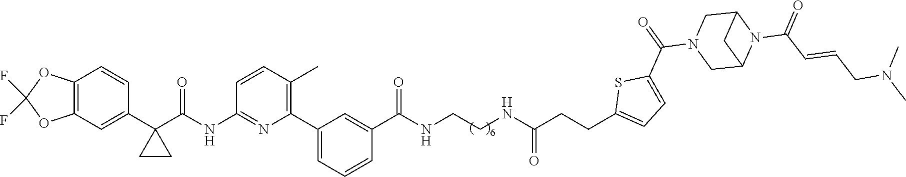

Example 359: (E)-3-(6-(1-(2,2-difluorobenzo[d][1,3]dioxol-5-yl)cyclopropane-1-carboxamido)-3-methylpyridin-2-yl)-N-(7-(3-(5-(6-(4-(dimethylamino)but-2-enoyl)-3,6-diazabicyclo[3.1.1]heptane-3-carbonyl)thiophen-2-yl)propanamido)heptyl)benzamide (QC192-156) White solid, 31% yield. ¹H NMR (400 MHz, Methanol-d₄) δ 7.99 (d, J=8.6 Hz, 1H), 7.86-7.78 (m, 3H), 7.56-7.47 (m, 2H), 7.39-7.29 (m, 2H), 7.25 (dd, J=8.3, 1.7 Hz, 1H), 7.15 (d, J=8.3 Hz, 1H), 6.80 (d, J=3.8 Hz, 1H), 6.73-6.64 (m, 1H), 6.50 (d, J=15.1 Hz, 1H), 4.71 (s, 1H), 4.47 (s, 1H), 4.37-3.79 (m, 6H), 3.29 (t, J=7.2 Hz, 2H), 3.09-3.02 (m, 4H), 2.88-2.70 (m, 7H), 2.46 (t, J=7.2 Hz, 2H), 2.20 (s, 3H), 1.66-1.15 (m, 15H). MS (ESI) [M+H]⁺=938.4.

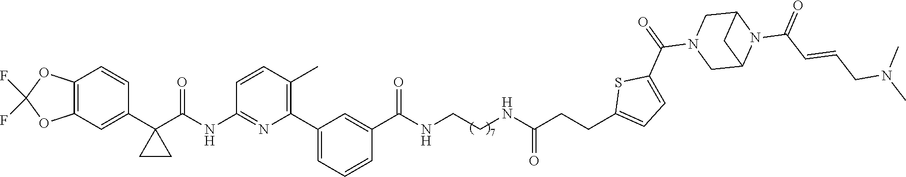

Example 360: (E)-3-(6-(1-(2,2-difluorobenzo[d][1,3]dioxol-5-yl)cyclopropane-1-carboxamido)-3-methylpyridin-2-yl)-N-(8-(3-(5-(6-(4-(dimethylamino)but-2-enoyl)-3,6-diazabicyclo[3.1.1]heptane-3-carbonyl)thiophen-2-yl)propanamido)octyl)benzamide (QC192-157) White solid, 27% yield. ¹H NMR (400 MHz, Methanol-d₄) δ 8.00 (d, J=8.6 Hz, 1H), 7.86-7.77 (m, 3H), 7.56-7.48 (m, 2H), 7.41-7.29 (m, 2H), 7.25 (dd, J=8.3, 1.7 Hz, 1H), 7.15 (d, J=8.3 Hz, 1H), 6.81 (d, J=3.8 Hz, 1H), 6.74-6.63 (m, 1H), 6.50 (d, J=14.8 Hz, 1H), 4.71 (s, 1H), 4.48 (s, 1H), 4.38-3.78 (m, 6H), 3.29 (t, J=7.4 Hz, 2H), 3.12-3.00 (m, 4H), 2.86-2.67 (m, 7H), 2.46 (t, J=7.3 Hz, 2H), 2.21 (s, 3H), 1.64-1.50 (m, 5H), 1.40-1.19 (m, 12H). MS (ESI) [M+H]⁺=952.4.

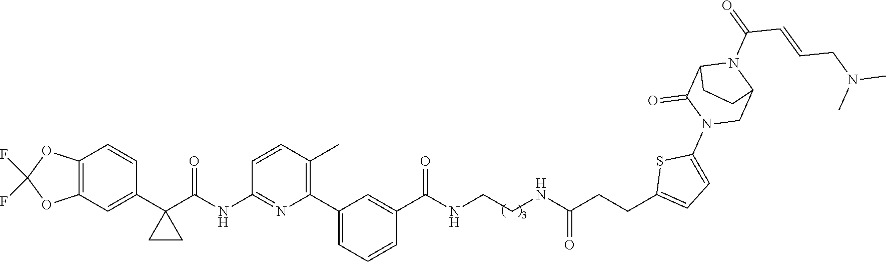

Example 361: (E)-3-(6-(1-(2,2-difluorobenzo[d][1,3]dioxol-5-yl)cyclopropane-1-carboxamido)-3-methylpyridin-2-yl)-N-(4-(3-(5-(8-(4-(dimethylamino)but-2-enoyl)-2-oxo-3,8-diazabicyclo[3.2.1]octan-3-yl)thiophen-2-yl)propanamido)butyl)benzamide (QC192-184) White solid, 30% yield. ¹H NMR (400 MHz, Methanol-d₄) δ 8.00 (d, J=8.5 Hz, 1H), 7.86-7.75 (m, 3H), 7.55-7.48 (m, 2H), 7.32 (d, J=1.7 Hz, 1H), 7.25 (dd, J=8.4, 1.7 Hz, 1H), 7.15 (d, J=8.2 Hz, 1H), 6.91-6.65 (m, 2H), 6.54 (d, J=3.9 Hz, 1H), 6.48-6.40 (m, 1H), 3.98-3.82 (m, 3H), 3.59 (dd, J=31.1, 11.6 Hz, 1H), 3.31-3.20 (m, 4H), 3.15-3.08 (m, 2H), 2.95 (t, J=7.3 Hz, 2H), 2.85-2.79 (m, 6H), 2.41 (t, J=7.3 Hz, 2H), 2.35-1.80 (m, 7H), 1.61 (q, J=4.1 Hz, 2H), 1.54-1.37 (m, 4H), 1.20 (q, J=4.1 Hz, 2H). MS (ESI) [M+H]⁺=896.4.

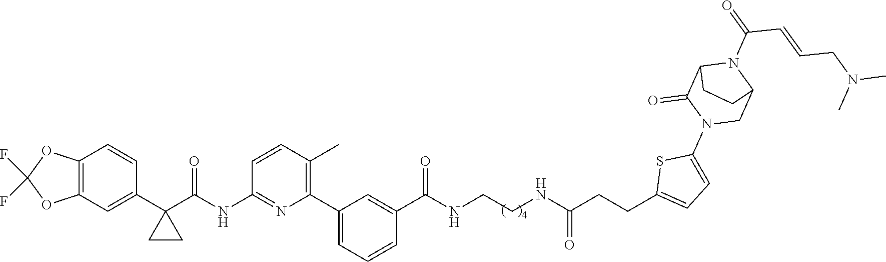

Example 362: (E)-3-(6-(1-(2,2-difluorobenzo[d][1,3]dioxol-5-yl)cyclopropane-1-carboxamido)-3-methylpyridin-2-yl)-N-(5-(3-(5-(8-(4-(dimethylamino)but-2-enoyl)-2-oxo-3,8-diazabicyclo[3.2.1]octan-3-yl)thiophen-2-yl)propanamido)pentyl)benzamide (QC192-178) White solid, 31% yield. ¹H NMR (400 MHz, Methanol-d₄) δ 7.99 (d, J=8.5 Hz, 1H), 7.86-7.74 (m, 3H), 7.55-7.45 (m, 2H), 7.31 (d, J=1.7 Hz, 1H), 7.25 (dd, J=8.4, 1.7 Hz, 1H), 7.15 (d, J=8.2 Hz, 1H), 6.90-6.65 (m, 2H), 6.55-6.40 (m, 2H), 4.00-3.81 (m, 3H), 3.61 (dd, J=27.0, 11.5 Hz, 1H), 3.30-3.20 (m, 4H), 3.08 (t, J=6.8 Hz, 2H), 2.92 (t, J=7.3 Hz, 2H), 2.88-2.78 (m, 6H), 2.43-1.81 (m, 9H), 1.65-1.36 (m, 6H), 1.31-1.14 (m, 4H). MS (ESI) [M+H]⁺=910.4.

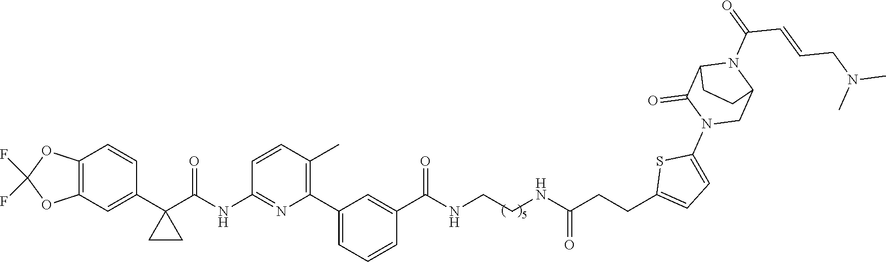

### Example 363: (E)-3-(6-(1-(2,2-difluorobenzo[d][1,3]dioxol-5-yl)cyclopropane-1-carboxamido)-3-methylpyridin-2-yl)-N-(6-(3-(5-(8-(4-(dimethylamino)but-2-enoyl)-2-oxo-3,8-diazabicyclo[3.2.1]octan-3-yl)thiophen-2-yl)propanamido)hexyl)benzamide (QC192-179)

White solid, 30% yield. ¹H NMR (400 MHz, Methanol-d₄) δ 8.00 (d, J=8.6 Hz, 1H), 7.85-7.73 (m, 3H), 7.56-7.47 (m, 2H), 7.32 (d, J=1.8 Hz, 1H), 7.25 (dd, J=8.4, 1.7 Hz, 1H), 7.15 (d, J=8.2 Hz, 1H), 6.93-6.65 (m, 2H), 6.56-6.42 (m, 2H), 4.01-3.85 (m, 3H), 3.61 (dd, J=25.6, 11.6 Hz, 1H), 3.35-3.20 (m, 4H), 3.07 (t, J=6.8 Hz, 2H), 2.94 (t, J=7.3 Hz, 2H), 2.87-2.79 (m, 6H), 2.45-1.84 (m, 9H), 1.65-1.16 (m, 12H). MS (ESI) [M+H]⁺=924.4.

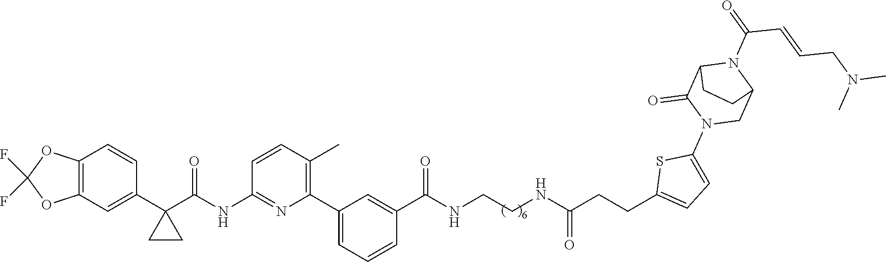

Example 364: (E)-3-(6-(1-(2,2-difluorobenzo[d][1,3]dioxol-5-yl)cyclopropane-1-carboxamido)-3-methylpyridin-2-yl)-N-(7-(3-(5-(8-(4-(dimethylamino)but-2-enoyl)-2-oxo-3,8-diazabicyclo[3.2.1]octan-3-yl)thiophen-2-yl)propanamido)heptyl)benzamide (QC192-180) White solid, 29% yield. ¹H NMR (400 MHz, Methanol-d₄) δ 8.00 (d, J=8.6 Hz, 1H), 7.87-7.74 (m, 3H), 7.56-7.46 (m, 2H), 7.32 (d, J=1.7 Hz, 1H), 7.25 (dd, J=8.2, 1.8 Hz, 1H), 7.16 (d, J=8.3 Hz, 1H), 6.94-6.65 (m, 2H), 6.58-6.41 (m, 2H), 4.02-3.83 (m, 3H), 3.61 (dd, J=24.3, 11.6 Hz, 1H), 3.34-3.20 (m, 4H), 3.05 (t, J=6.9 Hz, 2H), 2.94 (t, J=7.3 Hz, 2H), 2.88-2.78 (m, 6H), 2.45-1.79 (m, 9H), 1.65-1.47 (m, 4H), 1.42-1.12 (m, 10H). MS (ESI) [M+H]⁻=938.4.

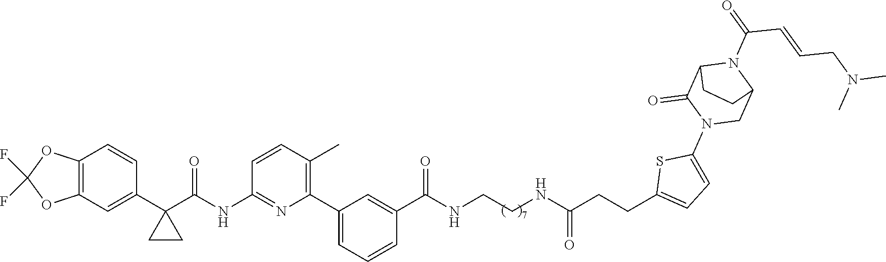

### Example 365: (E)-3-(6-(1-(2,2-difluorobenzo[d][1,3]dioxol-5-yl)cyclopropane-1-carboxamido)-3-methylpyridin-2-yl)-N-(8-(3-(5-(8-(4-(dimethylamino)but-2-enoyl)-2-oxo-3,8-diazabicyclo[3.2.1]octan-3-yl)thiophen-2-yl)propanamido)octyl)benzamide (QC192-181)

White solid, 29% yield. ¹H NMR (400 MHz, Methanol-d₄) δ 8.01 (d, J=8.5 Hz, 1H), 7.87-7.73 (m, 3H), 7.56-7.46 (m, 2H), 7.32 (d, J=1.7 Hz, 1H), 7.26 (dd, J=8.3, 1.7 Hz, 1H), 7.16 (d, J=8.2 Hz, 1H), 6.92-6.66 (m, 2H), 6.57-6.42 (m, 2H), 4.02-3.85 (m, 3H), 3.62 (dd, J=24.5, 11.5 Hz, 1H), 3.35-3.20 (m, 4H), 3.05 (t, J=6.9 Hz, 2H), 2.94 (t, J=7.3 Hz, 2H), 2.87-2.79 (m, 6H), 2.46-1.84 (m, 9H), 1.67-1.48 (m, 4H), 1.43-1.12 (m, 12H). MS (ESI) [M+H]⁺=952.4.

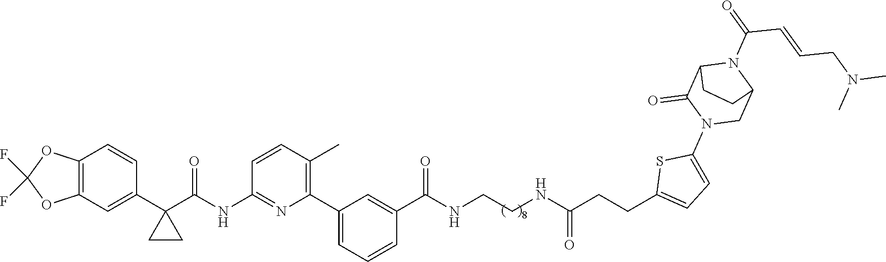

Example 366: (E)-3-(6-(1-(2,2-difluorobenzo[d][1,3]dioxol-5-yl)cyclopropane-1-carboxamido)-3-methylpyridin-2-yl)-N-(9-(3-(5-(8-(4-(dimethylamino)but-2-enoyl)-2-oxo-3,8-diazabicyclo[3.2.1]octan-3-yl)thiophen-2-yl)propanamido)nonyl)benzamide (QC192-182) White solid, 26% yield. ¹H NMR (400 MHz, Methanol-d₄) δ 8.01 (d, J=8.5 Hz, 1H), 7.86-7.74 (m, 3H), 7.56-7.47 (m, 2H), 7.32 (d, J=1.7 Hz, 1H), 7.26 (dd, J=8.2, 1.7 Hz, 1H), 7.15 (d, J=8.2 Hz, 1H), 6.92-6.65 (m, 2H), 6.57-6.40 (m, 2H), 4.02-3.80 (m, 3H), 3.61 (dd, J=24.3, 11.5 Hz, 1H), 3.36-3.20 (m, 4H), 3.04 (t, J=7.0 Hz, 2H), 2.94 (t, J=7.4 Hz, 2H), 2.87-2.79 (m, 6H), 2.44-1.80 (m, 9H), 1.66-1.47 (m, 4H), 1.44-1.11 (m, 14H). MS (ESI) [M+H]⁺=966.4.

Examples of OTUB1 covalent binders are set forth in table 1 below.

TABLE 1

| Ex- | | | |
|---|---|---|---|
| ample | Cpd | | Chemical |
| No. | ID | Structure | name |
| 1 | XS154- 91 | 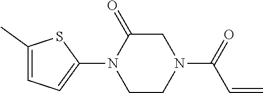 [SMILES: C=CC(=O)N1CCN(c2ccc(C)s2)C(=O)C1] | 4-acryloyl-1-(5- methylthiophen-2- yl)piperazin-2-one |
| 2 | XS154- 130 | 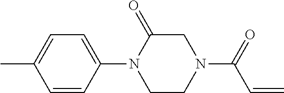 [SMILES: C=CC(=O)N1CCN(c2ccc(C)cc2)C(=O)C1] | 4-acryloyl-1-(p- tolyl)piperazin-2-one |
| 3 | XS154- 148 | 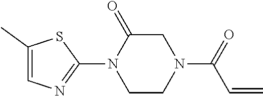 [SMILES: C=CC(=O)N1CCN(c2ncc(C)s2)C(=O)C1] | 4-acryloyl-1-(5- methylthiazol-2- yl)piperazin-2-one |
| 4 | XS154- 114 | 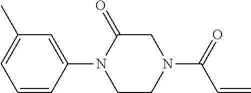 [SMILES: C=CC(=O)N1CCN(c2cccc(C)c2)C(=O)C1] | 4-acryloyl-1-(m- tolyl)piperazin-2-one |
| 5 | XS154- 149 | 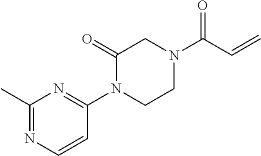 [SMILES: C=CC(=O)N1CCN(c2ccnc(C)n2)C(=O)C1] | 4-acryloyl-1-(2- methylpyrimidin-4- yl)piperazin-2-one |
| 6 | XS159- 13 | 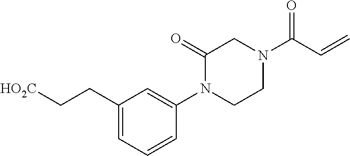 [SMILES: C=CC(=O)N1CCN(c2cccc(CCC(=O)O)c2)C(=O)C1] | 3-(3-(4-acryloyl-2- oxopiperazin-1- yl)phenyl)propanoic |
| 7 | XS159- 107 | 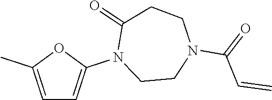 [SMILES: C=CC(=O)N1CCC(=O)N(c2ccc(C)o2)CC1] | 1-acryloyl-4-(5- methylfuran-2-yl)-1,4- diazepan-5-one |
| 8 | XS165- 30 | 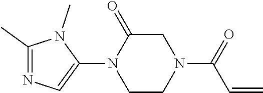 [SMILES: C=CC(=O)N1CCN(c2cnc(C)n2C)C(=O)C1] | 4-acryloyl-1-(1,2-dimethyl- 1H-imidazol-5-yl)piperazin- 2-one |
| 9 | XS159- 90 | 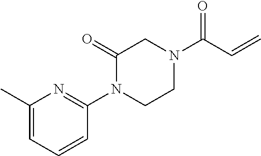 [SMILES: C=CC(=O)N1CCN(c2cccc(C)n2)C(=O)C1] | 4-acryloyl-1-(6- methylpyridin-2- yl)piperazin-2-one |
| 10 | XS165- 53 | 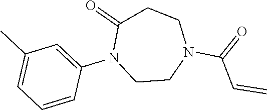 [SMILES: C=CC(=O)N1CCC(=O)N(c2cccc(C)c2)CC1] | 1-acryloyl-4-(m-tolyl)-1,4- diazepan-5-one |
| 11 | XS165- 38 | 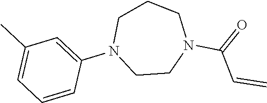 [SMILES: C=CC(=O)N1CCCN(c2cccc(C)c2)CC1] | 1-(4-(m-tolyl)-1,4-diazepan- 1-yl)prop-2-en-1-one |
| 12 | XS165- 33 | 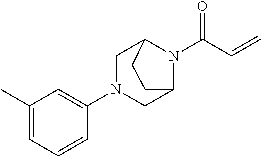 [SMILES: C=CC(=O)N1C2CCC1CN(c1cccc(C)c1)C2] | 1-(3-(m-tolyl)-3,8- diazabicyclo[3.2.1]octan-8- yl)prop-2-en-1-one |
| 13 | XS165- 54 | 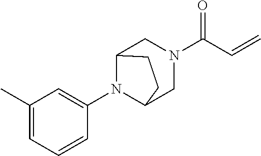 [SMILES: C=CC(=O)N1CC2CCC(C1)N2c1cccc(C)c1] | 1-(8-(m-tolyl)-3,8- diazabicyclo[3.2.1]octan-3- yl)prop-2-en-1-one |
| 14 | XS154- 184 | 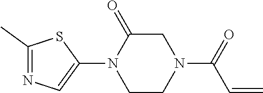 [SMILES: C=CC(=O)N1CCN(c2cnc(C)s2)C(=O)C1] | 4-acryloyl-1-(2- methylthiazol-5- yl)piperazin-2-one |
| 15 | XS165- 75 | 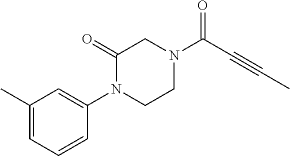 [SMILES: CC#CC(=O)N1CCN(c2cccc(C)c2)C(=O)C1] | 4-(but-2-ynoyl)-1-(m- tolyl)piperazin-2-one |
| 16 | XS165- 77 | 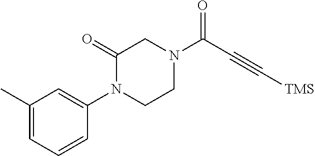 [SMILES: Cc1cccc(N2CCN(C(=O)C#C[Si](C)(C)C)CC2=O)c1] | 1-(m-tolyl)-4-(3- (trimethylsilyl)propioloyl) piperazin-2-one |
| 17 | XS165- 127 | 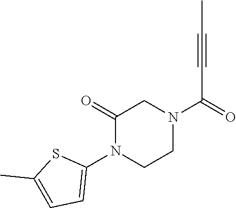 [SMILES: CC#CC(=O)N1CCN(c2ccc(C)s2)C(=O)C1] | 4-(but-2-ynoyl)-1-(5- methylthiophen-2- yl)piperazin-2-one |
| 18 | XS165- 106 | 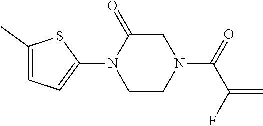 [SMILES: C=C(F)C(=O)N1CCN(c2ccc(C)s2)C(=O)C1] | 4-(2-fluoroacryloyl)-1-(5- methylthiophen-2- yl)piperazin-2-one |
| 19 | XS165- 112 | 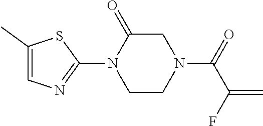 [SMILES: C=C(F)C(=O)N1CCN(c2ncc(C)s2)C(=O)C1] | 4-(2-fluoroacryloyl)-1-(5- methylthiazol-2- yl)piperazin-2-one |
| 20 | XS165- 97 | 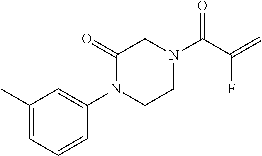 [SMILES: C=C(F)C(=O)N1CCN(c2cccc(C)c2)C(=O)C1] | 4-(2-fluoroacryloyl)-1-(m- tolyl)piperazin-2-one |
| 21 | XS165- 118 | 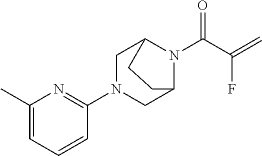 [SMILES: C=C(F)C(=O)N1C2CCC1CN(c1cccc(C)n1)C2] | 2-fluoro-1-(3-(6- methylpyridin-2-yl)-3,8- diazabicyclo[3.2.1]octan-8- yl)prop-2-en-1-one |
| 22 | XS165- 110 | 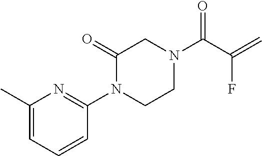 [SMILES: C=C(F)C(=O)N1CCN(c2cccc(C)n2)C(=O)C1] | 4-(2-fluoroacryloyl)-1-(6- methylpyridin-2- yl)piperazin-2-one |
| 23 | XS165- 119 | 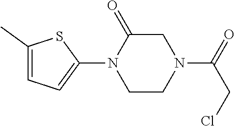 [SMILES: Cc1ccc(N2CCN(C(=O)CCl)CC2=O)s1] | 4-(2-chloroacetyl)-1-(5- methylthiophen-2- yl)piperazin-2-one |
| 24 | XS165- 123 | 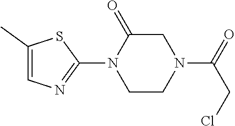 [SMILES: Cc1cnc(N2CCN(C(=O)CCl)CC2=O)s1] | 4-(2-chloroacetyl)-1-(5- methylthiazol-2- yl)piperazin-2-one |
| 25 | XS165- 126 | 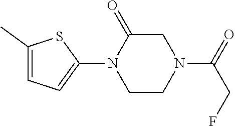 [SMILES: Cc1ccc(N2CCN(C(=O)CF)CC2=O)s1] | 4-(2-fluoroacetyl)-1-(5- methylthiophen-2- yl)piperazin-2-one |
| 26 | XS165- 120 | 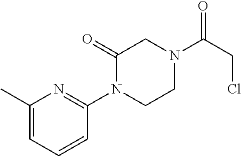 [SMILES: Cc1cccc(N2CCN(C(=O)CCl)CC2=O)n1] | 4-(2-chloroacetyl)-1-(6- methylpyridin-2- yl)piperazin-2-one |
| 27 | XS165- 121 | 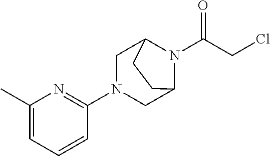 [SMILES: Cc1cccc(N2CC3CCC(C2)N3C(=O)CCl)n1] | 2-chloro-1-(3-(6- methylpyridin-2-yl)-3,8- diazabicyclo[3.2.1]octan-8- yl)ethan-1-one |
| 28 | XS165- 100 | 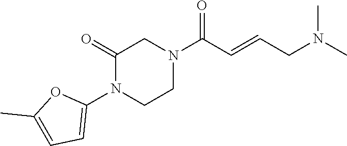 | (E)-4-(4- (dimethylamino)but-2- enoyl)-1-(5-methylfuran-2- yl)piperazin-2-one |
| 29 | XS165- 99 | 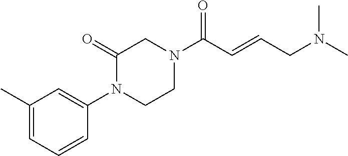 | (E)-4-(4- (dimethylamino)but-2- enoyl)-1-(m-tolyl)piperazin- 2-one |
| 30 | XS165- 113 | 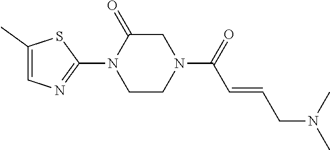 | (E)-4-(4- (dimethylamino)but-2- enoyl)-1-(5-methylthiazol-2- yl)piperazin-2-one |
| 31 | XS165- 109 | 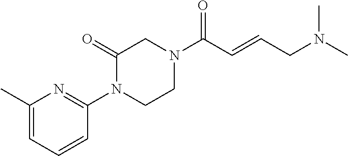 | (E)-4-(4- (dimethylamino)but-2- enoyl)-1-(6-methylpyridin- 2-yl)piperazin-2-one |
| 32 | XS165- 117 | 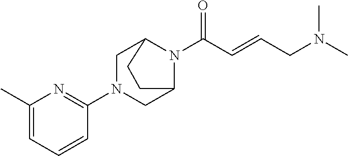 | (E)-4-(dimethylamino)-1-(3- (6-methylpyridin-2-yl)-3,8- diazabicyclo[3.2.1]octan-8- yl)but-2-en-1-one |
| 33 | XS165- 105 (MS51 05) | 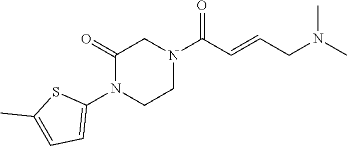 | (E)-4-(4- (dimethylamino)but-2- enoyl)-1-(5- methylthiophen-2- yl)piperazin-2-one |
| 34 | XS165- 154 | 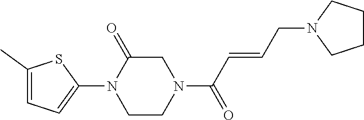 [SMILES: Cc1ccc(N2CCN(C(=O)/C=C/CN3CCCC3)CC2=O)s1] | (E)-1-(5-methylthiophen-2- yl)-4-(4-(pyrrolidin-1- yl)but-2-enoyl)piperazin-2- one |
| 35 | XS165- 170 | 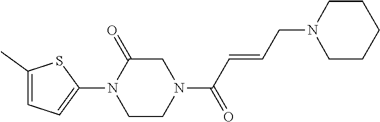 [SMILES: Cc1ccc(N2CCN(C(=O)/C=C/CN3CCCCC3)CC2=O)s1] | (E)-1-(5-methylthiophen-2- yl)-4-(4-(piperidin-1-yl)but- 2-enoyl)piperazin-2-one |
| 36 | XS165- 155 | 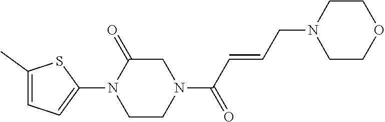 [SMILES: Cc1ccc(N2CCN(C(=O)/C=C/CN3CCOCC3)CC2=O)s1] | (E)-1-(5-methylthiophen-2- yl)-4-(4-morpholinobut-2- enoyl)piperazin-2-one |
| 37 | XS165- 172 | 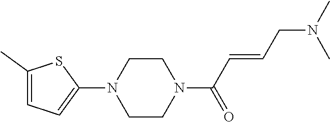 | (E)-4-(dimethylamino)-1-(4- (5-methylthiophen-2- yl)piperazin-1-yl)but-2-en- 1-one |
| 38 | XS165- 169 | 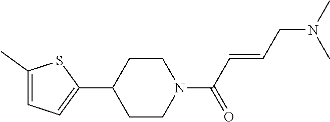 | (E)-4-(dimethylamino)-1-(4- (5-methylthiophen-2- yl)piperidin-1-yl)but-2-en- 1-one |
| 39 | XS165- 177 | 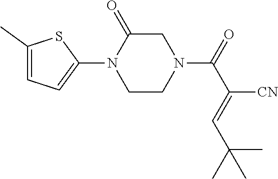 [SMILES: Cc1ccc(N2CCN(C(=O)/C(C#N)=C/C(C)(C)C)CC2=O)s1] | (E)-4,4-dimethyl-2-(4-(5- methylthiophen-2-yl)-3- oxopiperazine-1- carbonyl)pent-2-enenitrile |
| 40 | XS175- 45 | 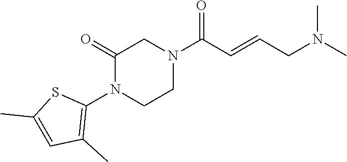 | (E)-4-(4- (dimethylamino)but-2- enoyl)-1-(3,5- dimethylthiophen-2- yl)piperazin-2-one |
| 41 | XS175- 46 | 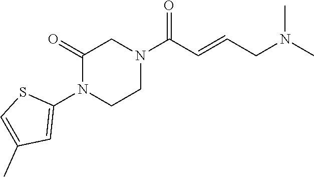 [SMILES: Cc1csc(N2CCN(C(=O)/C=C/CN(C)C)CC2=O)c1] | (E)-4-(4- (dimethylamino)but-2- enoyl)-1-(4- methylthiophen-2- yl)piperazin-2-one |
| 42 | XS175- 59 | 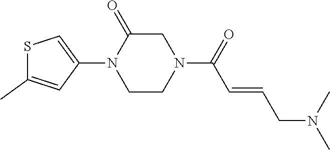 | (E)-4-(4- (dimethylamino)but-2- enoyl)-1-(5- methylthiophen-3- yl)piperazin-2-one |
| 43 | XS175- 63 | 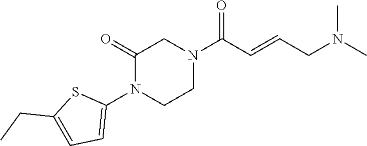 [SMILES: CCc1ccc(N2CCN(C(=O)/C=C/CN(C)C)CC2=O)s1] | (E)-4-(4- (dimethylamino)but-2- enoyl)-1-(5-ethylthiophen- 2-yl)piperazin-2-one |
| 44 | XS175- 64 | 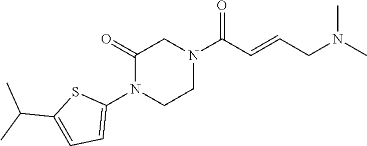 [SMILES: CC(C)c1ccc(N2CCN(C(=O)/C=C/CN(C)C)CC2=O)s1] | (E)-4-(4- (dimethylamino)but-2- enoyl)-1-(5- isopropylthiophen-2- yl)piperazin-2-one |
| 45 | XS175- 67 | 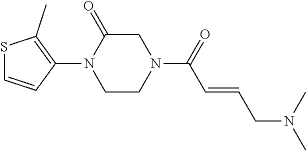 [SMILES: Cc1sccc1N1CCN(C(=O)/C=C/CN(C)C)CC1=O] | (E)-4-(4- (dimethylamino)but-2- enoyl)-1-(2- methylthiophen-3- yl)piperazin-2-one |
| 46 | XS175- 68 | 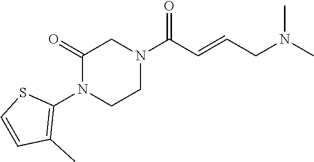 [SMILES: Cc1ccsc1N1CCN(C(=O)/C=C/CN(C)C)CC1=O] | (E)-4-(4- (dimethylamino)but-2- enoyl)-1-(3- methylthiophen-2- yl)piperazin-2-one |
| 47 | XS175- 70 | 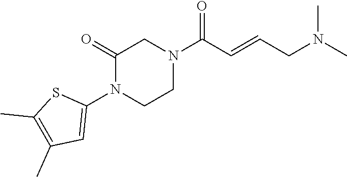 | (E)-4-(4- (dimethylamino)but-2- enoyl)-1-(4,5- dimethylthiophen-2- yl)piperazin-2-one |
| 48 | XS175- 71 | 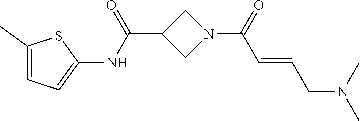 | (E)-1-(4- (dimethylamino)but-2- enoyl)-N-(5- methylthiophen-2- yl)azetidine-3-carboxamide |
| 49 | XS175- 76 | 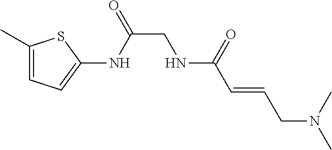 | (E)-4-(dimethylamino)-N- (2-((5-methylthiophen-2- yl)amino)-2-oxoethyl)but-2- |
| 50 | XS175- 110 | 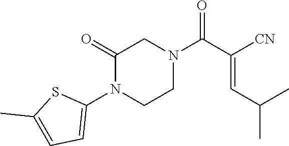 [SMILES: Cc1ccc(N2CCN(C(=O)/C(C#N)=C/C(C)C)CC2=O)s1] | (E)-4-methyl-2-(4-(5- methylthiophen-2-yl)-3- oxopiperazine-1- carbonyl)pent-2-enenitrile |
| 51 | XS175- 120 | 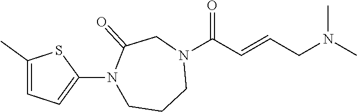 | (E)-4-(4- (dimethylamino)but-2- enoyl)-1-(5- methylthiophen-2-yl)-1,4- diazepan-2-one |
| 52 | XS175- 126 | 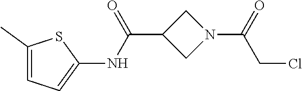 [SMILES: Cc1ccc(NC(=O)C2CN(C(=O)CCl)C2)s1] | 1-(2-chloroacetyl)-N-(5- methylthiophen-2- yl)azetidine-3-carboxamide |
| 53 | XS175- 132 | 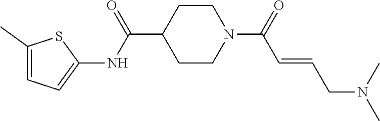 | (E)-1-(4- (dimethylamino)but-2- enoyl)-N-(5- methylthiophen-2- yl)piperidine-4- carboxamide |
| 54 | XS175- 133 | 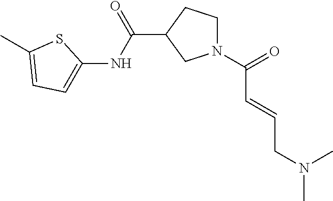 | (E)-1-(4- (dimethylamino)but-2- enoyl)-N-(5- methylthiophen-2- yl)pyrrolidine-3- carboxamide |
| 55 | XS175- 137 | 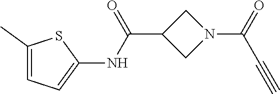 [SMILES: C#CC(=O)N1CC(C(=O)Nc2ccc(C)s2)C1] | N-(5-methylthiophen-2-yl)- 1-propioloylazetidine-3- carboxamide |
| 56 | XS175- 143 | 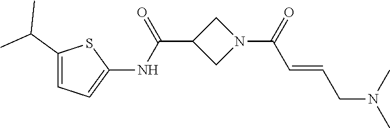 [SMILES: CC(C)c1ccc(NC(=O)C2CN(C(=O)/C=C/CN(C)C)C2)s1] | (E)-1-(4- (dimethylamino)but-2- enoyl)-N-(5- isopropylthiophen-2- yl)azetidine-3-carboxamide |
| 57 | XS175- 148 | 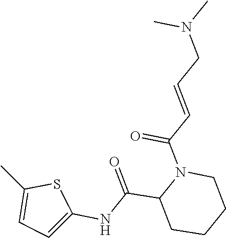 [SMILES: Cc1ccc(NC(=O)C2CCCCN2C(=O)/C=C/CN(C)C)s1] | (E)-1-(4- (dimethylamino)but-2- enoyl)-N-(5- methylthiophen-2- yl)piperidine-2- carboxamide |
| 58 | XS175- 149 | 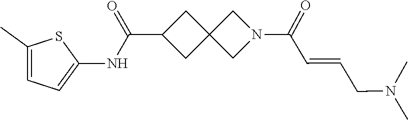 | (E)-2-(4- (dimethylamino)but-2- enoyl)-N-(5- methylthiophen-2-yl)-2- azaspiro[3.3]heptane-6- carboxamide |
| 59 | XS175- 158 | 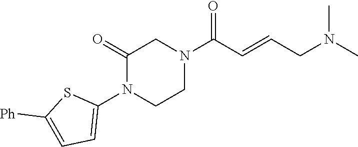 [SMILES: CN(C)C/C=C/C(=O)N1CCN(c2ccc(-c3ccccc3)s2)C(=O)C1] | (E)-4-(4- (dimethylamino)but-2- enoyl)-1-(5-phenylthiophen- 2-yl)piperazin-2- |
| 60 | XS175- 159 | 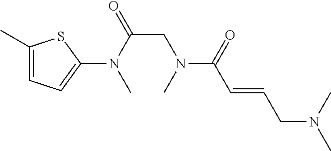 | (E)-4-(dimethylamino)-N- methyl-N-(2-(methyl(5- methylthiophen-2- yl)amino)-2-oxoethyl)but-2- enamide |
| 61 | XS175- 160 | 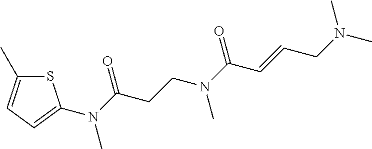 | (E)-4-(dimethylamino)-N- methyl-N-(3-(methyl(5- methylthiophen-2- yl)amino)-3-oxopropyl)but- 2-enamide |
| 62 | XS175- 173 | 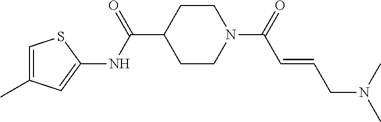 | (E)-1-(4- (dimethylamino)but-2- enoyl)-N-(4- methylthiophen-2- yl)piperidine-4- carboxamide |
| 63 | XS175- 174 | 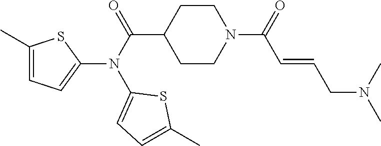 | (E)-1-(4- (dimethylamino)but-2- enoyl)-N,N-bis(5- methylthiophen-2- yl)piperidine-4- carboxamide |
| 64 | XS175- 175 | 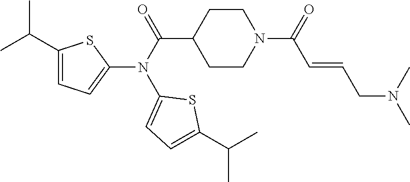 [SMILES: CC(C)c1ccc(N(C(=O)C2CCN(C(=O)/C=C/CN(C)C)CC2)c2ccc(C(C)C)s2)s1] | (E)-1-(4- (dimethylamino)but-2- enoyl)-N,N-bis(5- isopropylthiophen-2- yl)piperidine-4- carboxamide |
| 65 | XS175- 176 | 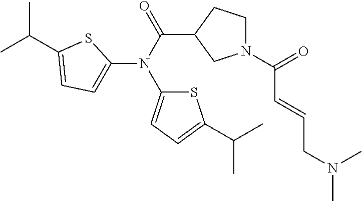 [SMILES: CC(C)c1ccc(N(C(=O)C2CCN(C(=O)/C=C/CN(C)C)C2)c2ccc(C(C)C)s2)s1] | (E)-1-(4- (dimethylamino)but-2- enoyl)-N,N-bis(5- isopropylthiophen-2- yl)pyrrolidine-3- carboxamide |
| 66 | XS175- 178 | 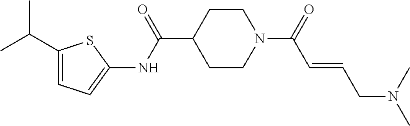 [SMILES: CC(C)c1ccc(NC(=O)C2CCN(C(=O)/C=C/CN(C)C)CC2)s1] | (E)-1-(4- (dimethylamino)but-2- enoyl)-N-(5- isopropylthiophen-2- yl)piperidine-4- carboxamide |
| 67 | XS175- 179 | 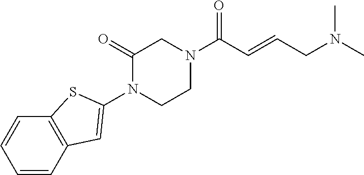 [SMILES: CN(C)C/C=C/C(=O)N1CCN(c2cc3ccccc3s2)C(=O)C1] | (E)-1-(benzo[b]thiophen-2- yl)-4-(4- (dimethylamino)but-2- enoyl)piperazin-2-one |
| 68 | XS175- 180 | 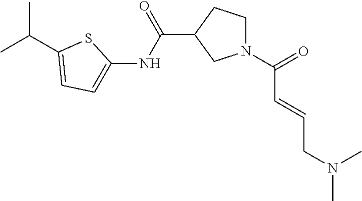 [SMILES: CC(C)c1ccc(NC(=O)C2CCN(C(=O)/C=C/CN(C)C)C2)s1] | (E)-1-(4- (dimethylamino)but-2- enoyl)-N-(5- isopropylthiophen-2- yl)pyrrolidine-3- carboxamide |
| 69 | XS175- 186 | 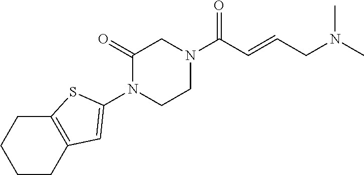 [SMILES: CN(C)C/C=C/C(=O)N1CCN(c2cc3c(s2)CCCC3)C(=O)C1] | (E)-4-(4- (dimethylamino)but-2- enoyl)-1-(4,5,6,7- tetrahydrobenzo[b]thiophen- 2-yl)piperazin-2-one |
| 70 | XS186- 3 | 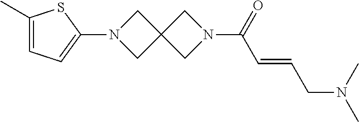 | (E)-4-(dimethylamino)-1-(6- (5-methylthiophen-2-yl)- 2,6-diazaspiro[3.3]heptan- 2-yl)but-2-en-1-one |
| 71 | XS185- 4 | 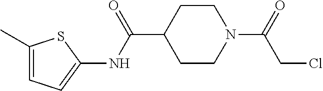 [SMILES: Cc1ccc(NC(=O)C2CCN(C(=O)CCl)CC2)s1] | 1-(2-chloroacetyl)-N-(5- methylthiophen-2- yl)piperidine-4- carboxamide |
| 72 | XS186- 5 | 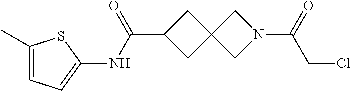 [SMILES: Cc1ccc(NC(=O)C2CC3(C2)CN(C(=O)CCl)C3)s1] | 2-(2-chloroacetyl)-N-(5- methylthiophen-2-yl)-2- azaspiro[3.3]heptane-6- carboxamide |
| 73 | XS185- 6 | 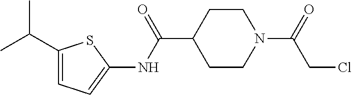 [SMILES: CC(C)c1ccc(NC(=O)C2CCN(C(=O)CCl)CC2)s1] | 1-(2-chloroacetyl)-N-(5- isopropylthiophen-2- yl)piperidine-4- carboxamide |
| 74 | XS185- 24 | 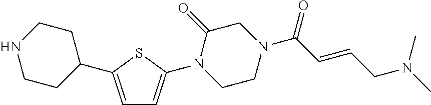 [SMILES: CN(C)C/C=C/C(=O)N1CCN(c2ccc(C3CCNCC3)s2)C(=O)C1] | (E)-4-(4- (dimethylamino)but-2- enoyl)-1-(5-(piperidin-4- yl)thiophen-2-yl)piperazin- 2-one |
| 75 | XS185- 29 | 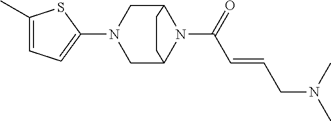 | (E)-4-(dimethylamino)-1-(3- (5-methylthiophen-2-yl)- 3,8- diazabicyclo[3.2.1]octan-8- yl)but-2-en-1-one |
| 76 | XS185- 43 | 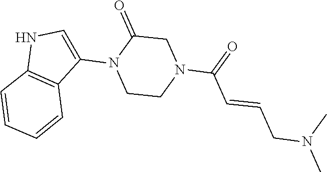 [SMILES: CN(C)C/C=C/C(=O)N1CCN(c2c[nH]c3ccccc23)C(=O)C1] | (E)-4-(4- (dimethylamino)but-2- enoyl)-1-(1H-indol-3- yl)piperazin-2-one |
| 77 | XS185- 46 | 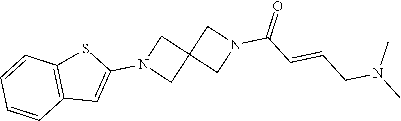 [SMILES: CN(C)C/C=C/C(=O)N1CC2(C1)CN(c1cc3ccccc3s1)C2] | (E)-1-(6-(benzo[b]thiophen- 2-yl)-2,6- diazaspiro[3.3]heptan-2-yl)- 4-(dimethylamino)but-2-en- 1-one |
| 78 | XS185- 59 | 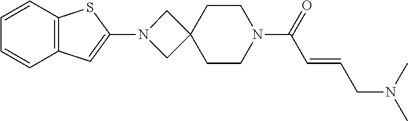 [SMILES: CN(C)C/C=C/C(=O)N1CCC2(CC1)CN(c1cc3ccccc3s1)C2] | (E)-1-(2-(benzo[b]thiophen- 2-yl)-2,7- diazaspiro[3.5]nonan-7-yl)- 4-(dimethylamino)but-2-en- 1-one |
| 79 | XS185- 64 | 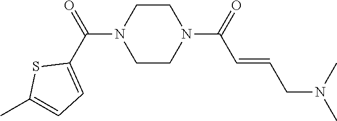 | (E)-4-(dimethylamino)-1-(4- (5-methylthiophene-2- carbonyl)piperazin-1- yl)but-2-en-1-one |
| 80 | XS185- 65 | 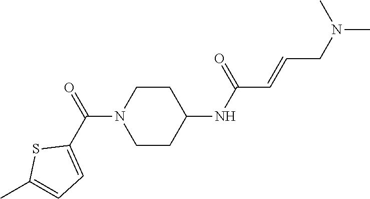 | (E)-4-(dimethylamino)-N- (1-(5-methylthiophene-2- carbonyl)piperidin-4- yl)but-2-enamide |
| 81 | XS185- 66 | 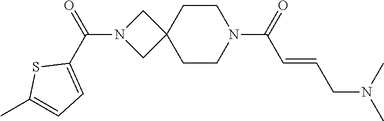 | (E)-4-(dimethylamino)-1-(2- (5-methylthiophene-2- carbonyl)-2,7- diazaspiro[3.5]nonan-7- yl)but-2-en-1-one |
| 82 | XS185- 67 | 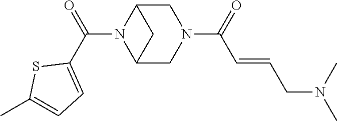 | (E)-4-(dimethylamino)-1-(6- (5-methylthiophene-2- carbonyl)-3,6- diazabicyclo[3.1.1]heptan- 3-yl)but-2-en-1-one |
| 83 | XS185- 69 | 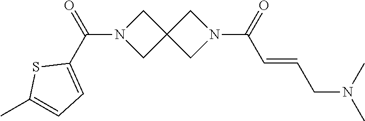 | (E)-4-(dimethylamino)-1-(6- (5-methylthiophene-2- carbonyl)-2,6- diazaspiro[3.3]heptan-2- yl)but-2-en-1-one |
| 84 | XS185- 70 | 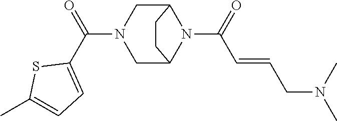 | (E)-4-(dimethylamino)-1-(3- (5-methylthiophene-2- carbonyl)-3,8- diazabicyclo[3.2.1]octan-8- yl)but-2-en-1-one |
| 85 | XS185- 71 | 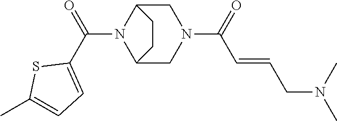 | (E)-4-(dimethylamino)-1-(8- (5-methylthiophene-2- carbonyl)-3,8- diazabicyclo[3.2.1]octan-3- yl)but-2-en-1-one |
| 86 | XS185- 72 | 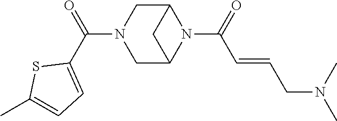 | (E)-4-(dimethylamino)-1-(3- (5-methylthiophene-2- carbonyl)-3,6- diazabicyclo[3.1.1]heptan- 6-yl)but-2-en-1-one |
| 87 | XS185- 75 | 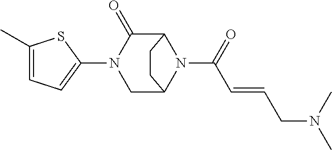 | (E)-8-(4- (dimethylamino)but-2- enoyl)-3-(5- methylthiophen-2-yl)-3,8- diazabicyclo[3.2.1]octan-2- one |
| 88 | XS185- 78 | 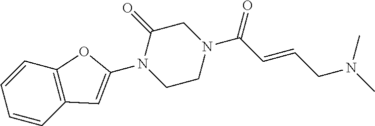 [SMILES: CN(C)C/C=C/C(=O)N1CCN(c2cc3ccccc3o2)C(=O)C1] | (E)-1-(benzofuran-2-yl)-4- (4-(dimethylamino)but-2- enoyl)piperazin-2-one |
| 89 | XS185- 86 | 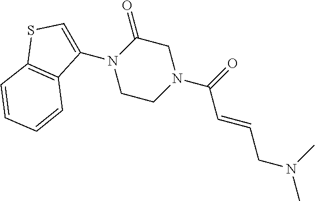 [SMILES: CN(C)C/C=C/C(=O)N1CCN(c2csc3ccccc23)C(=O)C1] | (E)-1-(benzo[b]thiophen-3- yl)-4-(4- (dimethylamino)but-2- enoyl)piperazin-2-one |
| 90 | XS185- 87 | 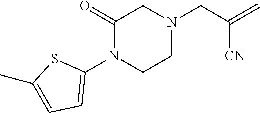 [SMILES: C=C(C#N)CN1CCN(c2ccc(C)s2)C(=O)C1] | 2-((4-(5-methylthiophen-2- yl)-3-oxopiperazin-1- yl)methyl)acrylonitrile |
| 91 | XS185- 90 | 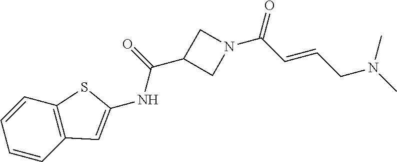 [SMILES: CN(C)C/C=C/C(=O)N1CC(C(=O)Nc2cc3ccccc3s2)C1] | (E)-N-(benzo[b]thiophen-2- yl)-1-(4- (dimethylamino)but-2- enoyl)azetidine-3- carboxamide |
| 92 | XS185- 91 | 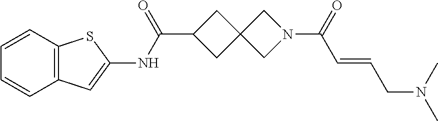 [SMILES: CN(C)C/C=C/C(=O)N1CC2(CC(C(=O)Nc3cc4ccccc4s3)C2)C1] | (E)-N-(benzo[b]thiophen-2- yl)-2-(4- (dimethylamino)but-2- enoyl)-2- azaspiro[3.3]heptane-6- carboxamide |
| 93 | XS185- 96 | 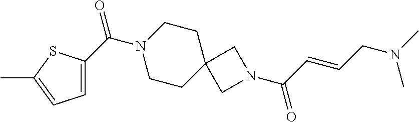 | (E)-4-(dimethylamino)-1-(7- (5-methylthiophene-2- carbonyl)-2,7- diazaspiro[3.5]nonan-2- yl)but-2-en-1-one |
| 94 | XS185- 97 | 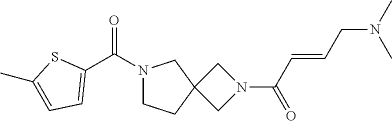 | (E)-4-(dimethylamino)-1-(6- (5-methylthiophene-2- carbonyl)-2,6- diazaspiro[3.4]octan-2- yl)but-2-en-1-one |
| 95 | XS185- 101 | 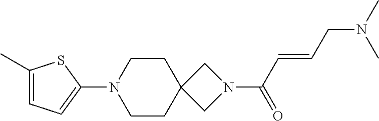 | (E)-4-(dimethylamino)-1-(7- (5-methylthiophen-2-yl)- 2,7-diazaspiro[3.5]nonan-2- yl)but-2-en-1-one |
| 96 | XS185- 109 | 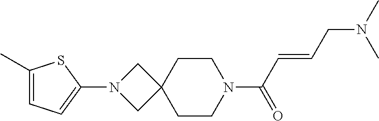 | (E)-4-(dimethylamino)-1-(2- (5-methylthiophen-2-yl)- 2,7-diazaspiro[3.5]nonan-7- yl)but-2-en-1-one |
| 97 | XS185- 113 | 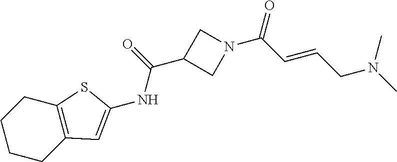 [SMILES: CN(C)C/C=C/C(=O)N1CC(C(=O)Nc2cc3c(s2)CCCC3)C1] | (E)-4-(dimethylamino)-1-(2- (5-methylthiophen-2-yl)- 2,7-diazaspiro[3.5]nonan-7- yl)but-2-en-1-one |
| 98 | XS185- 114 | 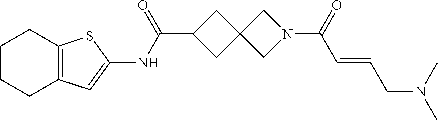 [SMILES: CN(C)C/C=C/C(=O)N1CC2(CC(C(=O)Nc3cc4c(s3)CCCC4)C2)C1] | (E)-2-(4- (dimethylamino)but-2- enoyl)-N-(4,5,6,7- tetrahydrobenzo[b]thiophen- 2-yl)-2- azaspiro[3.3]heptane-6- carboxamide |
| 99 | XS185- 116 | 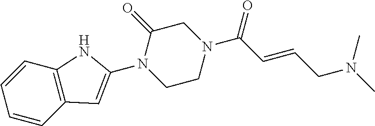 [SMILES: CN(C)C/C=C/C(=O)N1CCN(c2cc3ccccc3[nH]2)C(=O)C1] | (E)-4-(4- (dimethylamino)but-2- enoyl)-1-(1H-indol-2- yl)piperazin-2-one |
| 100 | XS185- 118 | 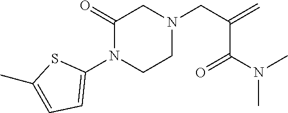 | N,N-dimethyl-2-((4-(5- methylthiophen-2-yl)-3- oxopiperazin-1- yl)methyl)acrylamide |
| 101 | XS185- 122 | 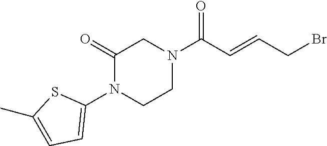 [SMILES: Cc1ccc(N2CCN(C(=O)/C=C/CBr)CC2=O)s1] | (E)-4-(4-bromobut-2- enoyl)-1-(5- methylthiophen-2- yl)piperazin-2-one |
| 102 | XS185- 131 | 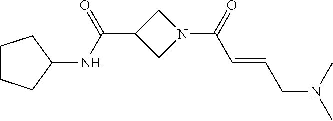 [SMILES: CN(C)C/C=C/C(=O)N1CC(C(=O)NC2CCCC2)C1] | (E)-N-cyclopentyl-1-(4- (dimethylamino)but-2- enoyl)azetidine-3- carboxamide |
| 103 | XS185- 134 | 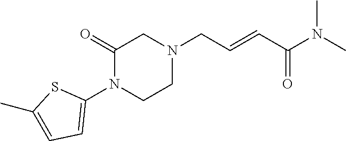 | (E)-N,N-dimethyl-4-(4-(5- methylthiophen-2-yl)-3- oxopiperazin-1-yl)but-2- enamide |
| 104 | XS185- 135 | 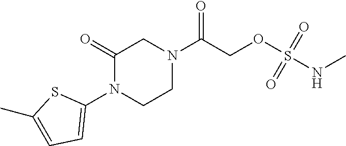 | (2-(4-(5-methylthiophen-2- yl)-3-oxopiperazin-1-yl)-2- oxoethyl methylsulfamate |
| 105 | XS185- 138 | 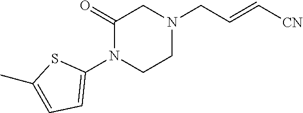 [SMILES: Cc1ccc(N2CCN(C/C=C/C#N)CC2=O)s1] | (E)-4-(4-(5-methylthiophen- 2-yl)-3-oxopiperazin-1- yl)but-2-enenitrile |
| 106 | XS185- 140 | 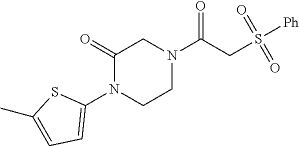 [SMILES: Cc1ccc(N2CCN(C(=O)CS(=O)(=O)c3ccccc3)CC2=O)s1] | 1-(5-methylthiophen-2-yl)- 4-(2- (phenylsulfonyl)acetyl) piperazin-2-one |
| 107 | XS185- 147 | 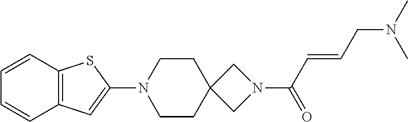 [SMILES: CN(C)C/C=C/C(=O)N1CC2(CCN(c3cc4ccccc4s3)CC2)C1] | (E)-1-(7-(benzo[b]thiophen- 2-yl)-2,7- diazaspiro[3.5]nonan-2-yl)- 4-(dimethylamino)but-2-en- 1-one |
| 108 | XS185- 149 | 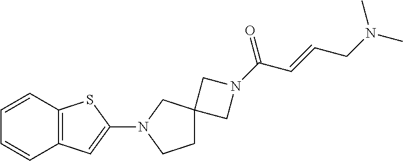 [SMILES: CN(C)C/C=C/C(=O)N1CC2(CCN(c3cc4ccccc4s3)C2)C1] | (E)-1-(6-(benzo[b]thiophen- 2-yl)-2,6- diazaspiro[3.4]octan-2-yl)- 4-(dimethylamino)but-2-en- 1-one |
| 109 | XS185- 150 | 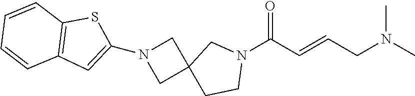 [SMILES: CN(C)C/C=C/C(=O)N1CCC2(C1)CN(c1cc3ccccc3s1)C2] | (E)-1-(2-(benzo[b]thiophen- 2-yl)-2,6- diazaspiro[3.4]octan-6-yl)- 4-(dimethylamino)but-2-en- 1-one |
| 110 | XS185- 171 | 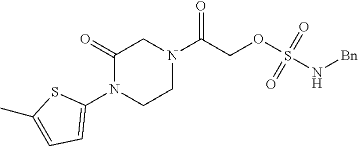 [SMILES: Cc1ccc(N2CCN(C(=O)COS(=O)(=O)NCc3ccccc3)CC2=O)s1] | 2-(4-(5-methylthiophen-2- yl)-3-oxopiperazin-1-yl)-2- oxoethyl benzylsulfamate |
| 111 | XS190- 9 | 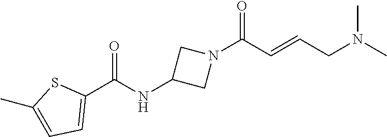 | (E)-N-(1-(4- (dimethylamino)but-2- enoyl)azetidin-3-yl)-5- methylthiophene-2- carboxamide |
| 112 | XS190- 27 | 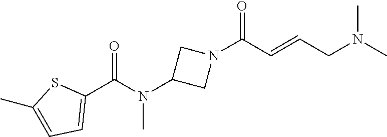 | (E)-N-(1-(4- (dimethylamino)but-2- enoyl)azetidin-3-yl)-N,5- dimethylthiophene-2- carboxamide |
| 113 | XS190- 38 | 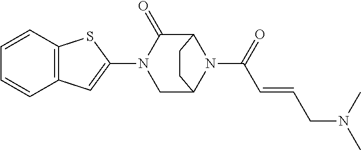 [SMILES: CN(C)C/C=C/C(=O)N1C2CCC1C(=O)N(c1cc3ccccc3s1)C2] | (E)-3-(benzo[b]thiophen-2- yl)-8-(4- (dimethylamino)but-2- enoyl)-3,8- diazabicyclo[3.2.1]octan-2- one |
| 114 | XS190- 44 | 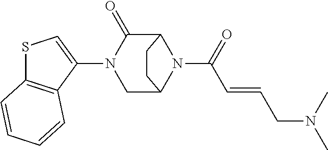 [SMILES: CN(C)C/C=C/C(=O)N1C2CCC1C(=O)N(c1csc3ccccc13)C2] | (E)-3-(benzo[b]thiophen-3- yl)-8-(4- (dimethylamino)but-2- enoyl)-3,8- diazabicyclo[3.2.1]octan-2- one |
| 115 | XS190- 45 | 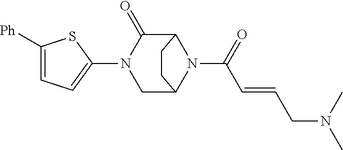 [SMILES: CN(C)C/C=C/C(=O)N1C2CCC1C(=O)N(c1ccc(-c3ccccc3)s1)C2] | (E)-8-(4- (dimethylamino)but-2- enoyl)-3-(5-phenylthiophen- 2-yl)-3,8- diazabicyclo[3.2.1]octan-2- one |
| 116 | XS190- 46 | 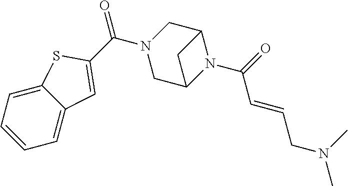 [SMILES: CN(C)C/C=C/C(=O)N1C2CC1CN(C(=O)c1cc3ccccc3s1)C2] | (E)-1-(3- (benzo[b]thiophene-2- carbonyl)-3,6- diazabicyclo[3.1.1]heptan- 6-yl)-4- (dimethylamino)but-2-en-1- one |
| 117 | XS190- 47 | 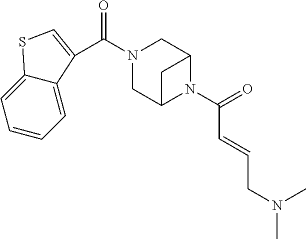 [SMILES: CN(C)C/C=C/C(=O)N1C2CC1CN(C(=O)c1csc3ccccc13)C2] | (E)-1-(3- (benzo[b]thiophene-3- carbonyl)-3,6- diazabicyclo[3.1.1]heptan- 6-yl)-4- (dimethylamino)but-2-en-1- one |
| 118 | XS190- 48 | 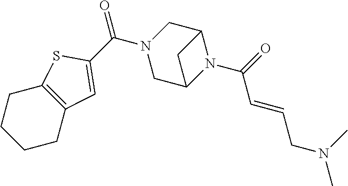 [SMILES: CN(C)C/C=C/C(=O)N1C2CC1CN(C(=O)c1cc3c(s1)CCCC3)C2] | (E)-4-(dimethylamino)-1-(3- (4,5,6,7- tetrahydrobenzo[b]thio- phene-2-carbonyl)-3,6- diazabicyclo[3.1.1]heptan- 6-yl)but-2-en-1-one |
| 119 | XS190- 49 | 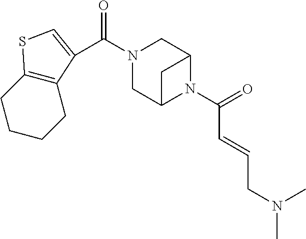 [SMILES: CN(C)C/C=C/C(=O)N1C2CC1CN(C(=O)c1csc3c1CCCC3)C2] | (E)-4-(dimethylamino)-1-(3- (4,5,6,7- tetrahydrobenzo[b]thio- phene-3-carbonyl)-3,6- diazabicyclo[3.1.1]heptan- 6-yl)but-2-en-1-one |
| 120 | XS190- 50 | 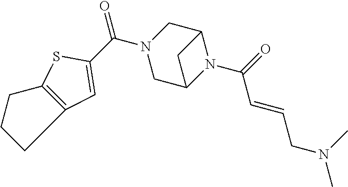 [SMILES: CN(C)C/C=C/C(=O)N1C2CC1CN(C(=O)c1cc3c(s1)CCC3)C2] | (E)-1-(3-(5,6-dihydro-4H- cyclopenta[b]thiophene-2- carbonyl)-3,6- diazabicyclo[3.1.1]heptan- 6-yl)-4- (dimethylamino)but-2-en-1- one |
| 121 | XS190- 59 | 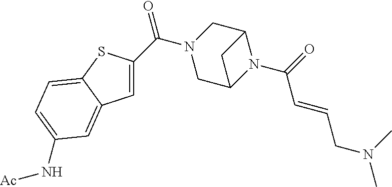 [SMILES: CC(=O)Nc1ccc2sc(C(=O)N3CC4CC(C3)N4C(=O)/C=C/CN(C)C)cc2c1] | (E)-N-(2-(6-(4- (dimethylamino)but-2- enoyl)-3,6- diazabicyclo[3.1.1]heptane- 3- carbonyl)benzo[b]thiophen- 5-yl)acetamide |
| 122 | XS190- 68 | 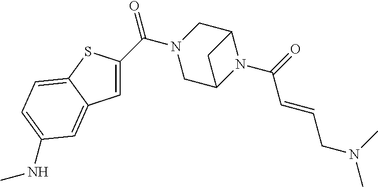 | (E)-4-(dimethylamino)-1-(3- (5- (methylamino)benzo[b] thiophene-2-carbonyl)-3,6- diazabicyclo[3.1.1]heptan- 6-yl)but-2-en-1-one |
| 123 | XS190- 69 | 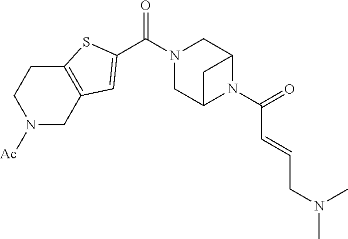 [SMILES: CC(=O)N1CCc2sc(C(=O)N3CC4CC(C3)N4C(=O)/C=C/CN(C)C)cc2C1] | (E)-1-(3-(5-acetyl-4,5,6,7- tetrahydrothieno[3,2- c]pyridine-2-carbonyl)-3,6- diazabicyclo[3.1.1]heptan- 6-yl)-4- (dimethylamino)but-2-en-1- one |
| 124 | XS190- 70 | 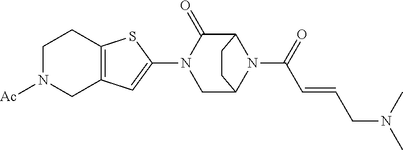 [SMILES: CC(=O)N1CCc2sc(N3CC4CCC(C3=O)N4C(=O)/C=C/CN(C)C)cc2C1] | (E)-3-(5-acetyl-4,5,6,7- tetrahydrothieno[3,2- c]pyridin-2-yl)-8-(4- (dimethylamino)but-2- enoyl)-3,8- diazabicyclo[3.2.1]octan-2- one |
| 125 | XS190- 75 | 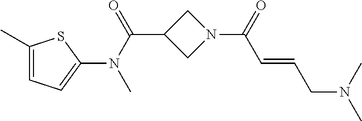 | (E)-1-(4- (dimethylamino)but-2- enoyl)-N-methyl-N-(5- methylthiophen-2- yl)azetidine-3-carboxamide |
| 126 | XS190- 75-2 | 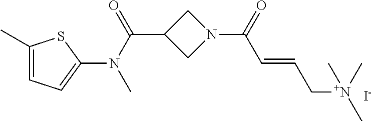 | (E)-N,N,N-trimethyl-4-(3- (methyl(5-methylthiophen- 2-yl)carbamoyl)azetidin-1- yl)-4-oxobut-2-en-1- aminium |
| 127 | XS190- 77 | 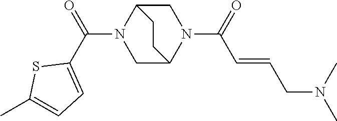 | (E)-4-(dimethylamino)-1-(5- (5-methylthiophene-2- carbonyl)-2,5- diazabicyclo[2.2.2]octan-2- yl)but-2-en-1-one |
| 128 | XS190- 80 | 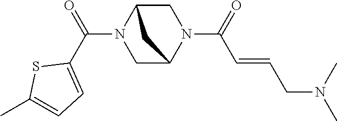 | (E)-4-(dimethylamino)-1- ((1S,4S)-5-(5- methylthiophene-2- carbonyl)-2,5- diazabicyclo[2.2.1]heptan- 2-yl)but-2-en-1-one |
| 129 | XS190- 81 | 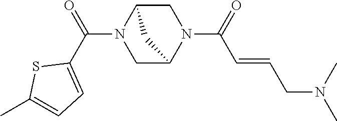 | (E)-4-(dimethylamino)-1- ((1R,4R)-5-(5- methylthiophene-2- carbonyl)-2,5- diazabicyclo [2.2.1]heptan- 2-yl)but-2-en-1-one |
| 130 | XS190- 126 | 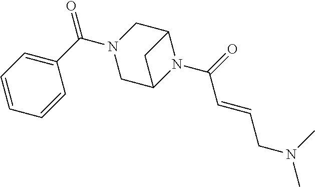 [SMILES: CN(C)C/C=C/C(=O)N1C2CC1CN(C(=O)c1ccccc1)C2] | (E)-1-(3-benzoyl-3,6- diazabicyclo[3.1.1]heptan- 6-yl)-4- (dimethylamino)but-2-en-1- one |
| 131 | XS190- 127 | 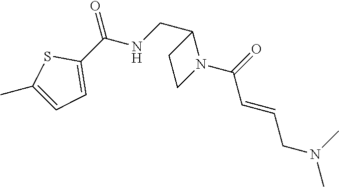 | (E)-N-((1-(4- (dimethylamino)but-2- enoyl)azetidin-2-yl)methyl)- 5-methylthiophene-2- carboxamide |
| 132 | XS190- 128 | 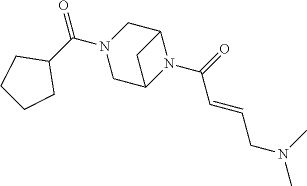 [SMILES: CN(C)C/C=C/C(=O)N1C2CC1CN(C(=O)C1CCCC1)C2] | (E)-1-(3- (cyclopentanecarbonyl)-3,6- diazabicyclo[3.1.1]heptan- 6-yl)-4- (dimethylamino)but-2-en-1- one |
| 133 | XS190- 129 | 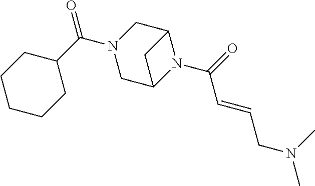 [SMILES: CN(C)C/C=C/C(=O)N1C2CC1CN(C(=O)C1CCCCC1)C2] | (E)-1-(3- (cyclohexanecarbonyl)-3,6- diazabicyclo[3.1.1]heptan- 6-yl)-4- (dimethylamino)but-2-en-1- one |
| 134 | XS190- 130 | 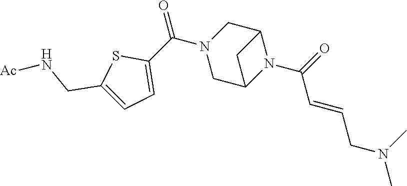 [SMILES: CC(=O)NCc1ccc(C(=O)N2CC3CC(C2)N3C(=O)/C=C/CN(C)C)s1] | (E)-N-((5-(6-(4- (dimethylamino)but-2- enoyl)-3,6- diazabicyclo[3.1.1]heptane- 3-carbonyl)thiophen-2- yl)methyl)acetamide |
| 135 | XS190- 137 | 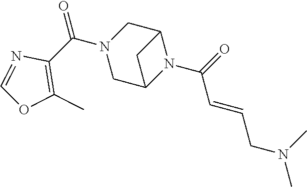 [SMILES: Cc1ocnc1C(=O)N1CC2CC(C1)N2C(=O)/C=C/CN(C)C] | (E)-4-(dimethylamino)-1-(3- (5-methyloxazole-4- carbonyl)-3,6- diazabicyclo[3.1.1]heptan- 6-yl)but-2-en-1-one |
| 136 | XS190- 138 | 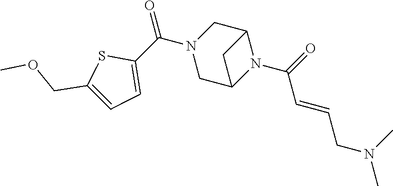 | (E)-4-(dimethylamino)-1-(3- (5- (methoxymethyl)thiophene- 2-carbonyl)-3,6- diazabicyclo[3.1.1]heptan- 6-yl)but-2-en-1-one |
| 137 | XS190- 144 | 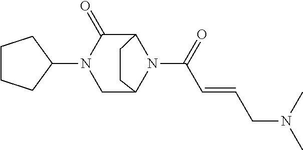 [SMILES: CN(C)C/C=C/C(=O)N1C2CCC1C(=O)N(C1CCCC1)C2] | (E)-3-cyclopentyl-8-(4- (dimethylamino)but-2- enoyl)-3,8- diazabicyclo[3.2.1]octan-2- one |
| 138 | XS190- 157 | 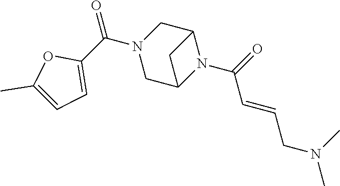 | (E)-4-(dimethylamino)-1-(3- (5-methylfuran-2- carbonyl)-3,6- diazabicyclo[3.1.1]heptan- 6-yl)but-2-en-1-one |
| 139 | XS190- 158 | 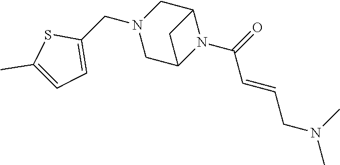 | (E)-4-(dimethylamino)-1-(3- ((5-methylthiophen-2- yl)methyl)-3,6- diazabicyclo[3.1.1]heptan- 6-yl)but-2-en-1-one |
| 140 | XS190- 170 | 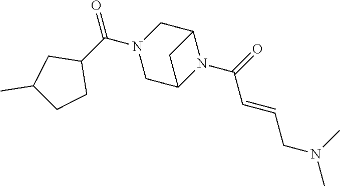 | (E)-4-(dimethylamino)-1-(3- (3-methylcyclopentane-1- carbonyl)-3,6- diazabicyclo[3.1.1]heptan- 6-yl)but-2-en-1-one |
| 141 | XS190- 181 | 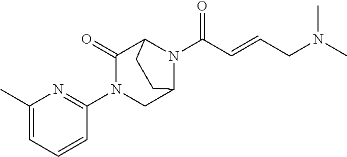 | ((E)-8-(4- (dimethylamino)but-2- enoyl)-3-(6-methylpyridin- 2-yl)-3,8- diazabicyclo[3.2.1]octan-2- one |
| 142 | XS190- 182 | 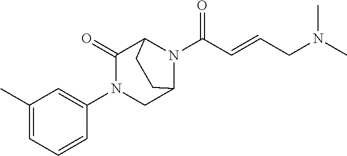 | (E)-8-(4- (dimethylamino)but-2- enoyl)-3-(m-tolyl)-3,8- diazabicyclo[3.2.1]octan-2- one |
| 143 | XS190- 183 | 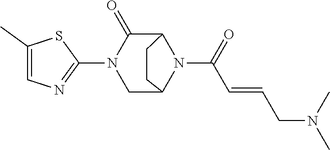 | (E)-8-(4- (dimethylamino)but-2- enoyl)-3-(5-methylthiazol-2- yl)-3,8- diazabicyclo[3.2.1]octan-2- one |
| 144 | XS190- 186 | 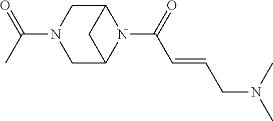 [SMILES: CC(=O)N1CC2CC(C1)N2C(=O)/C=C/CN(C)C] | ((E)-1-(3-acetyl-3,6- diazabicyclo[3.1.1]heptan- 6-yl)-4- (dimethylamino)but-2-en-1- one |
| 145 | XS197- 3 | 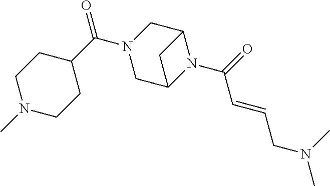 | (E)-4-(dimethylamino)-1-(3- (1-methylpiperidine-4- carbonyl)-3,6- diazabicyclo[3.1.1]heptan- 6-yl)but-2-en-1-one |
| 146 | XS197- 4 | 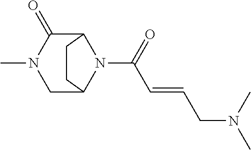 | (E)-8-(4- (dimethylamino)but-2- enoyl)-3-methyl-3,8- diazabicyclo[3.2.1]octan-2- one |
| 147 | XS197- 5 | 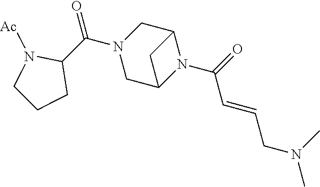 [SMILES: CC(=O)N1CCCC1C(=O)N1CC2CC(C1)N2C(=O)/C=C/CN(C)C] | (E)-8-(4- (dimethylamino)but-2- enoyl)-3-methyl-3,8- diazabicyclo[3.2.1]octan-2- one |
| 148 | XS197- 6 | 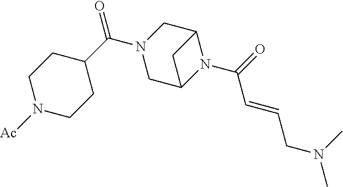 [SMILES: CC(=O)N1CCC(C(=O)N2CC3CC(C2)N3C(=O)/C=C/CN(C)C)CC1] | (E)-1-(3-(1- acetylpiperidine-4- carbonyl)-3,6- diazabicyclo[3.1.1]heptan- 6-yl)-4- (dimethylamino)but-2-en-1- one |
| 149 | XS197- 14 | 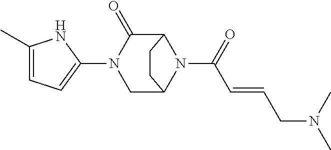 | (E)-8-(4- (dimethylamino)but-2- enoyl)-3-(5-methyl-1H- pyrrol-2-yl)-3,8- diazabicyclo[3.2.1]octan-2- one |
| 150 | XS197- 29 | 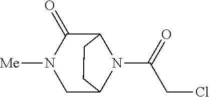 [SMILES: CN1CC2CCC(C1=O)N2C(=O)CCl] | 8-(2-chloroacetyl)-3- methyl-3,8- diazabicyclo[3.2.1]octan-2- one |
| 151 | XS197- 38 | 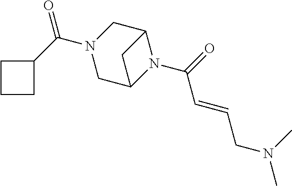 [SMILES: CN(C)C/C=C/C(=O)N1C2CC1CN(C(=O)C1CCC1)C2] | (E)-1-(3- (cyclobutanecarbonyl)-3,6- diazabicyclo[3.1.1]heptan- 6-yl)-4- (dimethylamino)but-2-en-1- one |
| 152 | XS197- 49 | 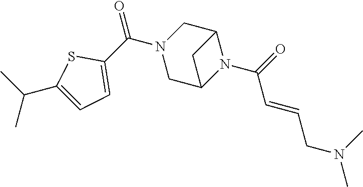 [SMILES: CC(C)c1ccc(C(=O)N2CC3CC(C2)N3C(=O)/C=C/CN(C)C)s1] | (E)-4-(dimethylamino)-1-(3- (5-isopropylthiophene-2- carbonyl)-3,6- diazabicyclo[3.1.1]heptan- 6-yl)but-2-en-1-one |
| 153 | XS197- 50 | 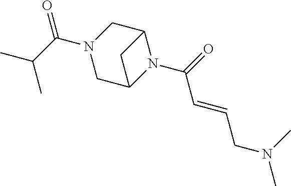 [SMILES: CC(C)C(=O)N1CC2CC(C1)N2C(=O)/C=C/CN(C)C] | (E)-4-(dimethylamino)-1-(3- isobutyryl-3,6- diazabicyclo[3.1.1]heptan- 6-yl)but-2-en-1-one |
| 154 | XS197- 70 | 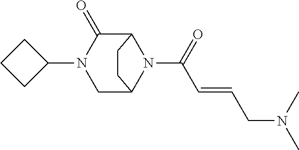 [SMILES: CN(C)C/C=C/C(=O)N1C2CCC1C(=O)N(C1CCC1)C2] | (E)-3-cyclobutyl-8-(4- (dimethylamino)but-2- enoyl)-3,8- diazabicyclo[3.2.1]octan-2- one |
| 155 | XS197- 72 | 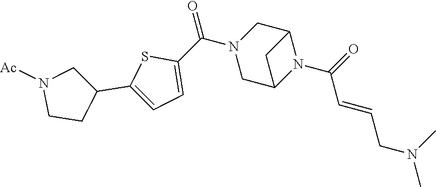 [SMILES: CC(=O)N1CCC(c2ccc(C(=O)N3CC4CC(C3)N4C(=O)/C=C/CN(C)C)s2)C1] | (E)-1-(3-(5-(1- acetylpyrrolidin-3- yl)thiophene-2-carbonyl)- 3,6- diazabicyclo[3.1.1]heptan- 6-yl)-4- (dimethylamino)but-2-en-1- one |
| 156 | XS197- 72 | 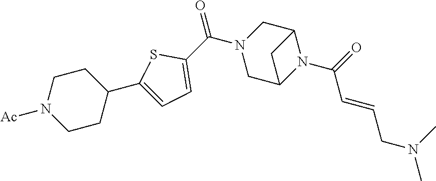 [SMILES: CC(=O)N1CCC(c2ccc(C(=O)N3CC4CC(C3)N4C(=O)/C=C/CN(C)C)s2)CC1] | (E)-1-(3-(5-(1- acetylpiperidin-4- yl)thiophene-2-carbonyl)- 3,6- diazabicyclo[3.1.1]heptan- 6-yl)-4- (dimethylamino)but-2-en-1- one |
| 157 | XS197- 73 | 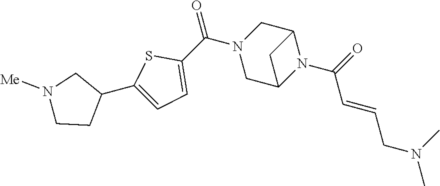 [SMILES: CN(C)C/C=C/C(=O)N1C2CC1CN(C(=O)c1ccc(C3CCN(C)C3)s1)C2] | (E)-4-(dimethylamino)-1-(3- (5-(1-methylpyrrolidin-3- yl)thiophene-2-carbonyl)- 3,6- diazabicyclo[3.1.1]heptan- 6-yl)but-2-en-1-one |
| 158 | XS197- 74 | 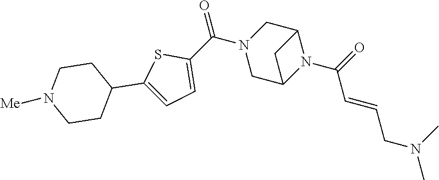 [SMILES: CN(C)C/C=C/C(=O)N1C2CC1CN(C(=O)c1ccc(C3CCN(C)CC3)s1)C2] | (E)-4-(dimethylamino)-1-(3- (5-(1-methylpiperidin-4- yl)thiophene-2-carbonyl)- 3,6- diazabicyclo[3.1.1]heptan- 6-yl)but-2-en-1-one |
| 159 | XS197- 85 | 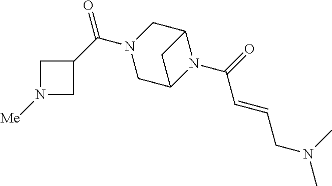 [SMILES: CN(C)C/C=C/C(=O)N1C2CC1CN(C(=O)C1CN(C)C1)C2] | (E)-4-(dimethylamino)-1-(3- (1-methylazetidine-3- carbonyl)-3,6- diazabicyclo[3.1.1]heptan- 6-yl)but-2-en-1-one |
| 160 | XS197- 93 | 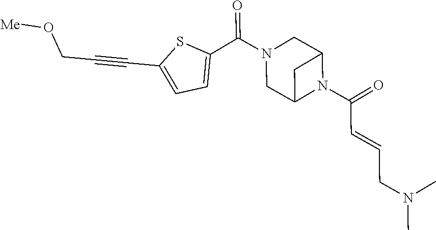 [SMILES: COCC#Cc1ccc(C(=O)N2CC3CC(C2)N3C(=O)/C=C/CN(C)C)s1] | (E)-4-(dimethylamino)-1-(3- (5-(3-methoxyprop-1-yn-1- yl)thiophene-2-carbonyl)- 3,6- diazabicyclo[3.1.1]heptan- 6-yl)but-2-en-1-one |
| 161 | XS197- 94 | 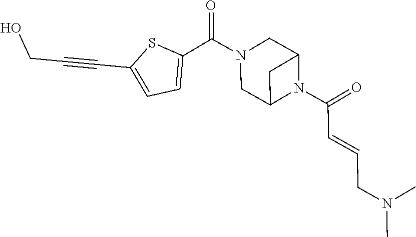 [SMILES: CN(C)C/C=C/C(=O)N1C2CC1CN(C(=O)c1ccc(C#CCO)s1)C2] | (E)-4-(dimethylamino)-1-(3- (5-(3-hydroxyprop-1-yn-1- yl)thiophene-2-carbonyl)- 3,6- diazabicyclo[3.1.1]heptan- 6-yl)but-2-en-1-one |
| 162 | XS197- 96 | 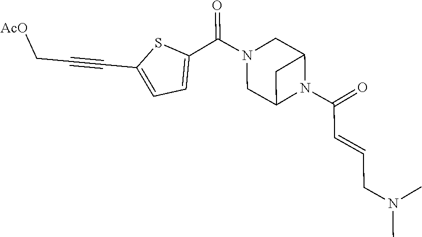 [SMILES: CC(=O)OCC#Cc1ccc(C(=O)N2CC3CC(C2)N3C(=O)/C=C/CN(C)C)s1] | (E)-3-(5-(6-(4- (dimethylamino)but-2- enoyl)-3,6- diazabicyclo[3.1.1]heptane- 3-carbonyl)thiophen-2- yl)prop-2-yn-1-yl acetate |
| 163 | XS197- 133 | 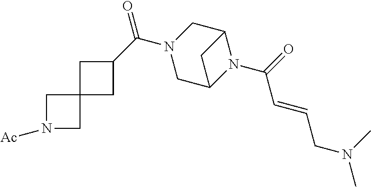 [SMILES: CC(=O)N1CC2(CC(C(=O)N3CC4CC(C3)N4C(=O)/C=C/CN(C)C)C2)C1] | (E)-1-(3-(2-acetyl-2- azaspiro[3.3]heptane-6- carbonyl)-3,6- diazabicyclo[3.1.1]heptan- 6-yl)-4- (dimethylamino)but-2-en-1- one |
| 164 | XS197- 145 | 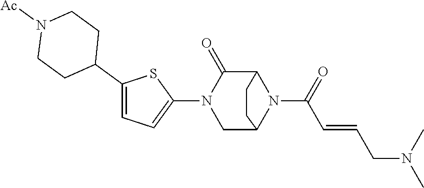 [SMILES: CC(=O)N1CCC(c2ccc(N3CC4CCC(C3=O)N4C(=O)/C=C/CN(C)C)s2)CC1] | (E)-3-(5-(1-acetylpiperidin- 4-yl)thiophen-2-yl)-8-(4- (dimethylamino)but-2- enoyl)-3,8- diazabicyclo[3.2.1]octan-2- one |
| 165 | XS197- 176 | 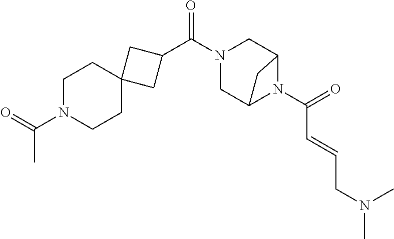 [SMILES: CC(=O)N1CCC2(CC1)CC(C(=O)N1CC3CC(C1)N3C(=O)/C=C/CN(C)C)C2] | (E)-1-(3-(7-acetyl-7- azaspiro[3.5]nonane-2- carbonyl)-3,6- diazabicyclo[3.1.1]heptan- 6-yl)-4- (dimethylamino)but-2-en-1- one |
| 166 | XS197- 185 | 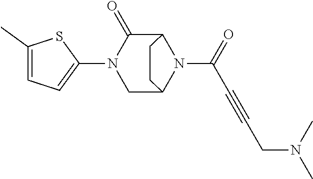 | 8-(4-(dimethylamino)but-2- ynoyl)-3-(5- methylthiophen-2-yl)-3,8- diazabicyclo[3.2.1]octan-2- one |
| 167 | XS197- 186 | 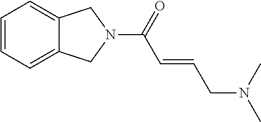 [SMILES: CN(C)C/C=C/C(=O)N1Cc2ccccc2C1] | (E)-4-(dimethylamino)-1- (isoindolin-2-yl)but-2-en-1- one |
| 168 | XS209- 21 | 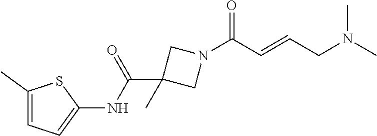 | (E)-1-(4- (dimethylamino)but-2- enoyl)-3-methyl-N-(5- methylthiophen-2- yl)azetidine-3-carboxamide |
| 169 | XS209- 22 | 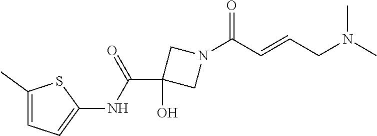 | (E)-1-(4- (dimethylamino)but-2- cnoyl)-3-hydroxy-N-(5- methylthiophen-2- yl)azetidine-3-carboxamide |
| 170 | XS209- 23 | 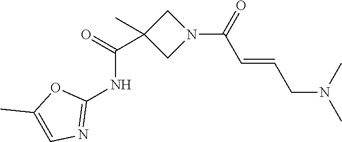 | (E)-1-(4- (dimethylamino)but-2- enoyl)-3-methyl-N-(5- methyloxazol-2- yl)azetidine-3-carboxamide |
| 171 | XS209- 24 | 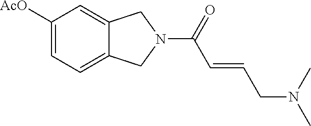 [SMILES: CC(=O)Oc1ccc2c(c1)CN(C(=O)/C=C/CN(C)C)C2] | (E)-2-(4- (dimethylamino)but-2- enoyl)isoindolin-5-yl acetate |
| 172 | XS209- 25 | 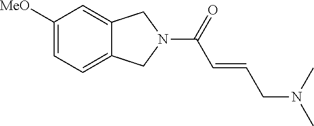 [SMILES: COc1ccc2c(c1)CN(C(=O)/C=C/CN(C)C)C2] | (E)-4-(dimethylamino)-1-(5- methoxyisoindolin-2-yl)but- 2-en-1-one |
| 173 | XS209- 26 | 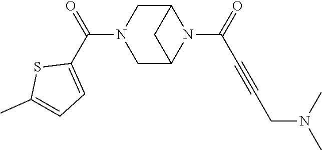 | 4-(dimethylamino)-1-(3-(5- methylthiophene-2- carbonyl)-3,6- diazabicyclo[3.1.1]heptan- 6-yl)but-2-yn-1-one |
| 174 | XS209- 27 | 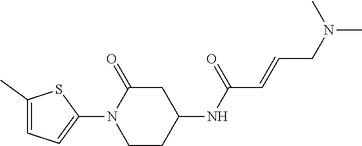 | (E)-4-(dimethylamino)-N- (1-(5-methylthiophen-2-yl)- 2-oxopiperidin-4-yl)but-2- enamide |
| 175 | XS209- 28 | 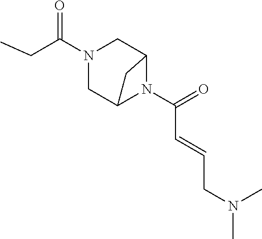 [SMILES: CCC(=O)N1CC2CC(C1)N2C(=O)/C=C/CN(C)C] | (E)-4-(dimethylamino)-1-(3- propionyl-3,6- diazabicyclo[3.1.1]heptan- 6-yl)but-2-en-1-one |
| 176 | XS209- 30 | 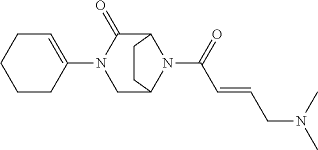 [SMILES: CN(C)C/C=C/C(=O)N1C2CCC1C(=O)N(C1=CCCCC1)C2] | (E)-3-(cyclohex-1-en-1-yl)- 8-(4-(dimethylamino)but-2- enoyl)-3,8- diazabicyclo[3.2.1]octan-2- one |
| 177 | XS209- 38 | 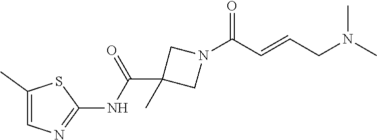 | (E)-1-(4- (dimethylamino)but-2- enoyl)-3-methyl-N-(5- methylthiazol-2- yl)azetidine-3-carboxamide |
| 178 | XS209- 39 | 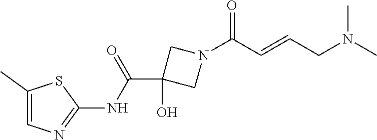 | (E)-1-(4- (dimethylamino)but-2- enoyl)-3-hydroxy-N-(5- methylthiazol-2- yl)azetidine-3-carboxamide |
| 179 | XS209- 40 | 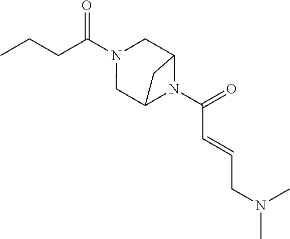 [SMILES: CCCC(=O)N1CC2CC(C1)N2C(=O)/C=C/CN(C)C] | (E)-1-(3-butyryl-3,6- diazabicyclo[3.1.1]heptan- 6-yl)-4- (dimethylamino)but-2-en-1- one |
| 180 | XS209- 53 | 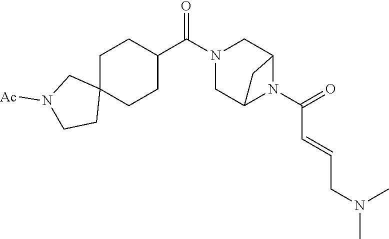 [SMILES: CC(=O)N1CCC2(CCC(C(=O)N3CC4CC(C3)N4C(=O)/C=C/CN(C)C)CC2)C1] | (E)-1-(3-(2-acetyl-2- azaspiro[4.5]decane-8- carbonyl)-3,6- diazabicyclo[3.1.1]heptan- 6-yl)-4- (dimethylamino)but-2-en-1- one |
| 181 | XS209- 54 | 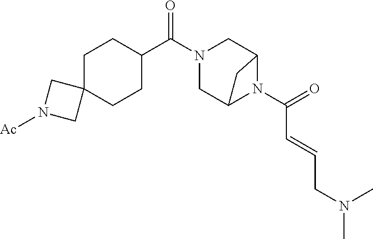 [SMILES: CC(=O)N1CC2(CCC(C(=O)N3CC4CC(C3)N4C(=O)/C=C/CN(C)C)CC2)C1] | (E)-1-(3-(2-acetyl-2- azaspiro[3.5]nonane-7- carbonyl)-3,6- diazabicyclo[3.1.1]heptan- 6-yl)-4- (dimethylamino)but-2-en-1- one |
| 182 | XS209- 55 | 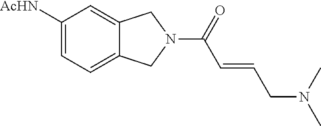 [SMILES: CC(=O)Nc1ccc2c(c1)CN(C(=O)/C=C/CN(C)C)C2] | (E)-N-(2-(4- (dimethylamino)but-2- enoyl)isoindolin-5- yl)acetamide |
| 183 | XS209- 57 | 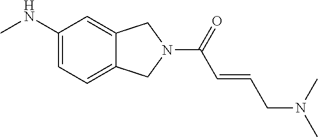 | (E)-4-(dimethylamino)-1-(5- (methylamino)isoindolin-2- yl)but-2-en-1-one |
| 184 | XS209- 60 | 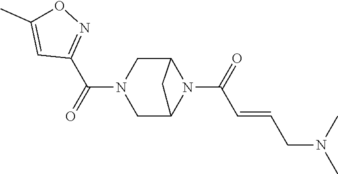 | (E)-4-(dimethylamino)-1-(5- (methylamino)isoindolin-2- yl)but-2-en-1-one |
| 185 | XS209- 62 | 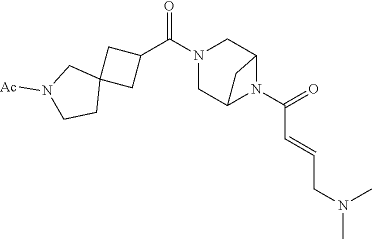 [SMILES: CC(=O)N1CCC2(CC(C(=O)N3CC4CC(C3)N4C(=O)/C=C/CN(C)C)C2)C1] | (E)-1-(3-(6-acetyl-6- azaspiro[3.4]octane-2- carbonyl)-3,6- diazabicyclo[3.1.1]heptan- 6-yl)-4- (dimethylamino)but-2-en-1- one |
| 186 | XS209- 65 | 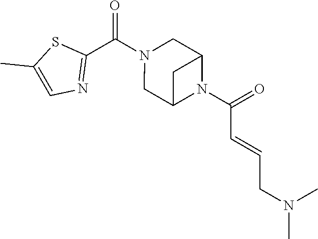 | (E)-4-(dimethylamino)-1-(3- (5-methylthiazole-2- carbonyl)-3,6- diazabicyclo[3.1.1]heptan- 6-yl)but-2-en-1-one |
| 187 | XS209- 74 | 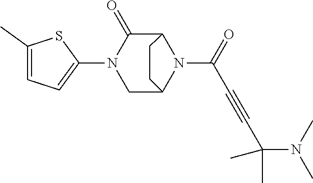 | 8-(4-(dimethylamino)-4- methylpent-2-ynoyl)-3-(5- methylthiophen-2-yl)-3,8- diazabicyclo[3.2.1]octan-2- one |
| 188 | XS209- 75 | 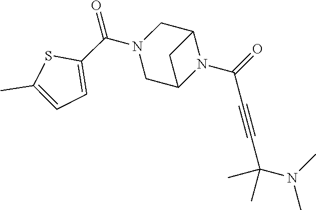 | 4-(dimethylamino)-4- methyl-1-(3-(5- methylthiophene-2- carbonyl)-3,6- diazabicyclo[3.1.1]heptan- 6-yl)pent-2-yn-1-one |
| 189 | XS209- 92 | 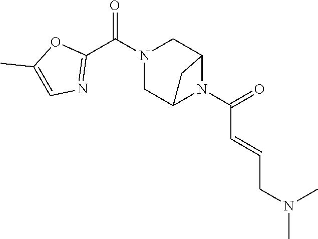 | (E)-4-(dimethylamino)-1-(3- (5-methyloxazole-2- carbonyl)-3,6- diazabicyclo[3.1.1]heptan- 6-yl)but-2-en-1-one |
| 190 | XS209- 99 | 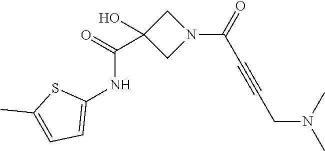 | 1-(4-(dimethylamino)but-2- ynoyl)-3-hydroxy-N-(5- methylthiophen-2- yl)azetidine-3-carboxamide |
| 191 | XS209- 100 | 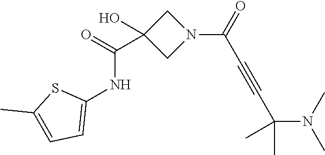 | 1-(4-(dimethylamino)-4- methylpent-2-ynoyl)-3- hydroxy-N-(5- methylthiophen-2- yl)azetidine-3-carboxamide |
| 192 | XS209- 101 | 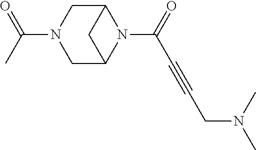 [SMILES: CC(=O)N1CC2CC(C1)N2C(=O)C#CCN(C)C] | 1-(3-acetyl-3,6- diazabicyclo[3.1.1]heptan- 6-yl)-4- (dimethylamino)but-2-yn-1- one |
| 193 | XS209- 122 | 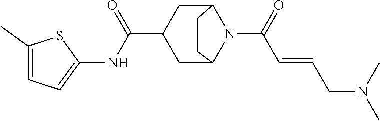 | (E)-8-(4- (dimethylamino)but-2- enoyl)-N-(5- methylthiophen-2-yl)-8- azabicyclo[3.2.1]octane-3- carboxamide |
| 194 | XS209- 139 | 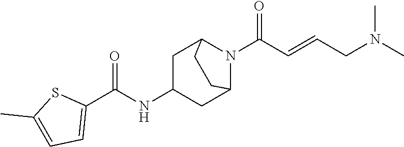 | (E)-N-(8-(4- (dimethylamino)but-2- enoyl)-8- azabicyclo[3.2.1]octan-3- yl)-5-methylthiophene-2- carboxamide |
| 195 | XS209- 140 | 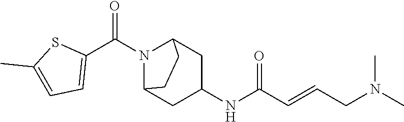 | (E)-4-(dimethylamino)-N- (8-(5-methylthiophene-2- carbonyl)-8- azabicyclo[3.2.1]octan-3- yl)but-2-enamide |
| 196 | XS209- 165 | 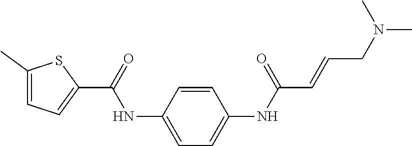 | (E)-N-(4-(4- (dimethylamino)but-2- enamido)phenyl)-5- methylthiophene-2- carboxamide |
| 197 | XS209- 166 | 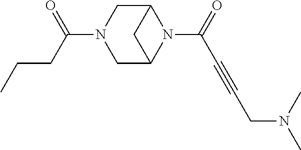 [SMILES: CCCC(=O)N1CC2CC(C1)N2C(=O)C#CCN(C)C] | Example 197 1-(3-butyryl- 3,6- diazabicyclo[3.1.1]heptan- 6-yl)-4- (dimethylamino)but-2-yn-1- one |
| 198 | XS209- 167 | 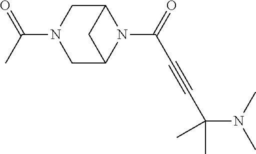 [SMILES: CC(=O)N1CC2CC(C1)N2C(=O)C#CC(C)(C)N(C)C] | 1-(3-acetyl-3,6- diazabicyclo[3.1.1]heptan- 6-yl)-4-(dimethylamino)-4- methylpent-2-yn-1-one |
| 199 | XS209- 168 | 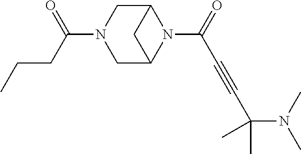 [SMILES: CCCC(=O)N1CC2CC(C1)N2C(=O)C#CC(C)(C)N(C)C] | 1-(3-butyryl-3,6- diazabicyclo[3.1.1]heptan- 6-yl)-4-(dimethylamino)-4- methylpent-2-yn-1-one |
| 200 | XS209- 174 | 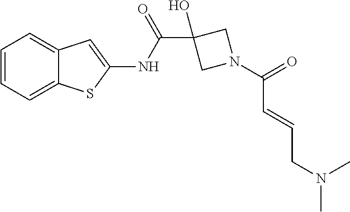 [SMILES: CN(C)C/C=C/C(=O)N1CC(O)(C(=O)Nc2cc3ccccc3s2)C1] | 1-(3-butyryl-3,6- diazabicyclo[3.1.1]heptan- 6-yl)-4-(dimethylamino)-4- methylpent-2-yn-1-one |
| 201 | XS209- 175 | 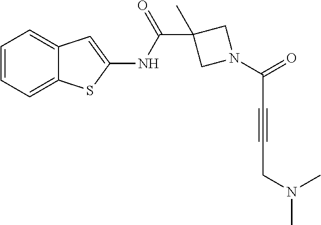 [SMILES: CN(C)CC#CC(=O)N1CC(C)(C(=O)Nc2cc3ccccc3s2)C1] | N-(benzo[b]thiophen-2-yl)- 1-(4-(dimethylamino)but-2- ynoyl)-3-methylazetidine-3- carboxamide |
| 202 | XS209- 176 | 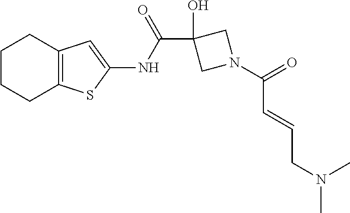 [SMILES: CN(C)C/C=C/C(=O)N1CC(O)(C(=O)Nc2cc3c(s2)CCCC3)C1] | (E)-1-(4- (dimethylamino)but-2- enoyl)-3-hydroxy-N- (4,5,6,7- tetrahydrobenzo[b] thiophen-2-yl)azetidine-3- carboxamide |
| 203 | XS209- 184 | 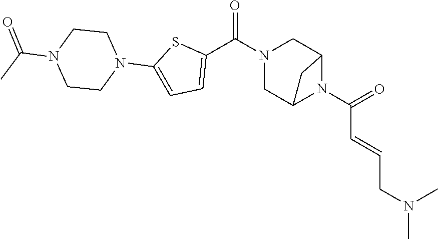 [SMILES: CC(=O)N1CCN(c2ccc(C(=O)N3CC4CC(C3)N4C(=O)/C=C/CN(C)C)s2)CC1] | (E)-1-(3-(5-(4- acetylpiperazin-1- yl)thiophene-2-carbonyl)- 3,6- diazabicyclo[3.1.1]heptan- 6-yl)-4- (dimethylamino)but-2-en-1- one |
| 204 | XS209- 185 | 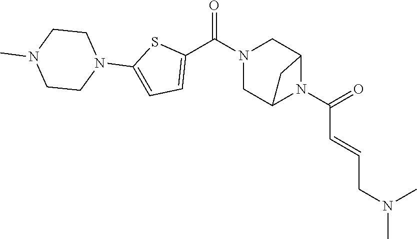 | (E)-4-(dimethylamino)-1-(3- (5-(4-methylpiperazin-1- yl)thiophene-2-carbonyl)- 3,6- diazabicyclo[3.1.1]heptan- 6-yl)but-2-en-1-one |
| 205 | XS224- 6 | 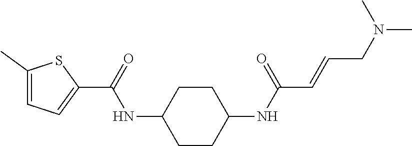 | (E)-N-(4-(4- (dimethylamino)but-2- enamido)cyclohexyl)-5- methylthiophene-2- carboxamide |
| 206 | XS224- 106 | 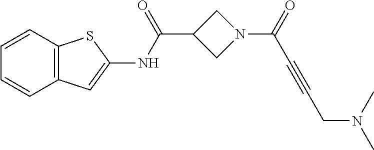 [SMILES: CN(C)CC#CC(=O)N1CC(C(=O)Nc2cc3ccccc3s2)C1] | N-(benzo[b]thiophen-2-yl)- 1-(4-(dimethylamino)but-2- ynoyl)azetidine-3- carboxamide |
| 207 | XS224- 107 | 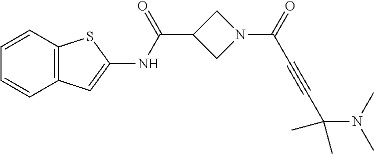 [SMILES: CN(C)C(C)(C)C#CC(=O)N1CC(C(=O)Nc2cc3ccccc3s2)C1] | N-(benzo[b]thiophen-2-yl)- 1-(4-(dimethylamino)-4- methylpent-2- ynoyl)azetidine-3- carboxamide |
| 208 | XS224- 108 | 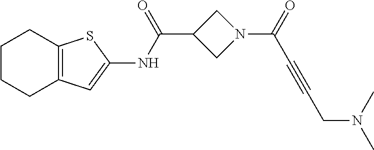 [SMILES: CN(C)CC#CC(=O)N1CC(C(=O)Nc2cc3c(s2)CCCC3)C1] | 1-(4-(dimethylamino)but-2- ynoyl)-N-(4,5,6,7- tetrahydrobenzo[b] thiophen-2-yl)azetidine-3- carboxamide |
| 209 | XS224- 109 | 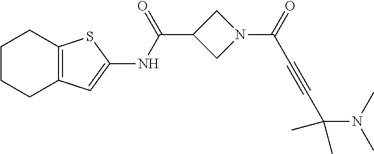 [SMILES: CN(C)C(C)(C)C#CC(=O)N1CC(C(=O)Nc2cc3c(s2)CCCC3)C1] | 1-(4-(dimethylamino)-4- methylpent-2-ynoyl)-N- (4,5,6,7- tetrahydrobenzo[b] thiophen-2-yl)azetidine-3- carboxamide |
| 210 | XS224- 110 | 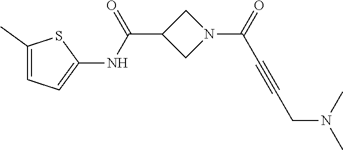 | 1-(4-(dimethylamino)but-2- ynoyl)-N-(5- methylthiophen-2- yl)azetidine-3-carboxamide |
| 211 | XS224- 111 | 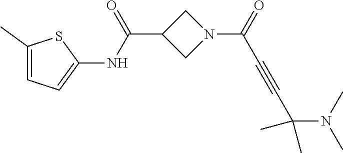 | 1-(4-(dimethylamino)-4- methylpent-2-ynoyl)-N-(5- methylthiophen-2- yl)azetidine-3-carboxamide |
| 212 | XS224- 116 | 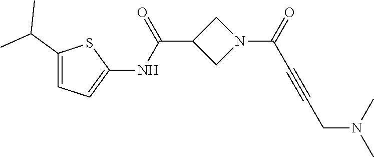 [SMILES: CC(C)c1ccc(NC(=O)C2CN(C(=O)C#CCN(C)C)C2)s1] | 1-(4-(dimethylamino)but-2- ynoyl)-N-(5- isopropylthiophen-2- yl)azetidine-3-carboxamide |
| 213 | XS224- 117 | 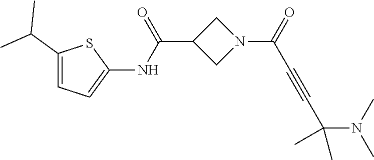 [SMILES: CC(C)c1ccc(NC(=O)C2CN(C(=O)C#CC(C)(C)N(C)C)C2)s1] | 1-(4-(dimethylamino)-4- methylpent-2-ynoyl)-N-(5- isopropylthiophen-2- yl)azetidine-3-carboxamide |
| 214 | XS224- 118 | 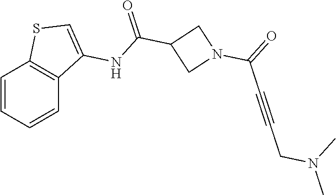 [SMILES: CN(C)CC#CC(=O)N1CC(C(=O)Nc2csc3ccccc23)C1] | 214 N-(benzo[b]thiophen-3- yl)-1-(4- (dimethylamino)but-2- ynoyl)azetidine-3- carboxamide |
| 215 | XS224- 119 | 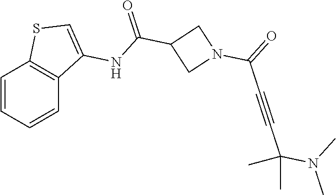 [SMILES: CN(C)C(C)(C)C#CC(=O)N1CC(C(=O)Nc2csc3ccccc23)C1] | N-(benzo[b]thiophen-3-yl)- 1-(4-(dimethylamino)-4- methylpent-2- ynoyl)azetidine-3- carboxamide |
| 216 | XS224- 143 | 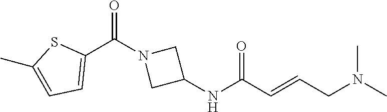 | (E)-4-(dimethylamino)-N- (1-(5-methylthiophene-2- carbonyl)azetidin-3-yl)but- 2-enamide |
| 217 | XS224- 144 | 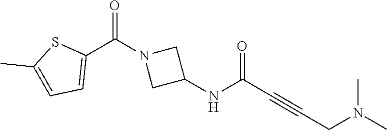 | 4-(dimethylamino)-N-(1-(5- methylthiophene-2- carbonyl)azetidin-3-yl)but- 2-ynamide |
| 218 | XS224- 145 | 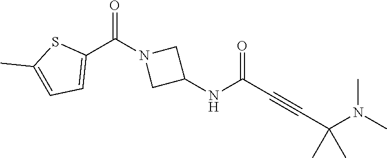 | 4-(dimethylamino)-4- methyl-N-(1-(5- methylthiophene-2- carbonyl)azetidin-3- yl)pent-2-ynamide |
| 219 | XS224- 147 | 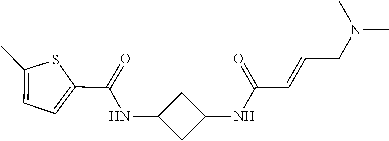 | (E)-N-(3-(4- (dimethylamino)but-2- enamido)cyclobutyl)-5- methylthiophene-2- carboxamide |
| 220 | XS224- 148 | 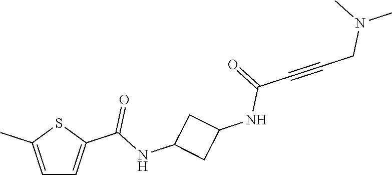 | N-(3-(4- (dimethylamino)but-2- ynamido)cyclobutyl)-5- methylthiophene-2- carboxamide |
| 221 | XS224- 149 | 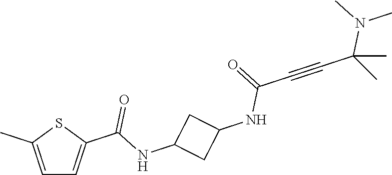 | N-(3-(4-(dimethylamino)-4- methylpent-2- ynamido)cyclobutyl)-5- methylthiophene-2- carboxamide |
| 222 | XS224- 150 | 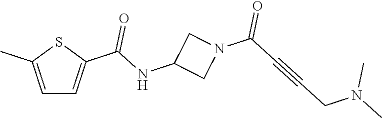 | N-(1-(4- (dimethylamino)but-2- ynoyl)azetidin-3-yl)-5- methylthiophene-2- carboxamide |
| 223 | XS224- 154 | 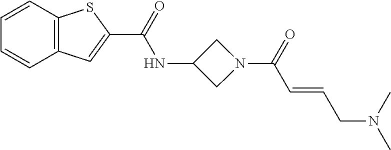 [SMILES: CN(C)C/C=C/C(=O)N1CC(NC(=O)c2cc3ccccc3s2)C1] | (E)-N-(1-(4- (dimethylamino)but-2- enoyl)azetidin-3- yl)benzo[b]thiophene-2- carboxamide |
| 224 | XS224- 155 | 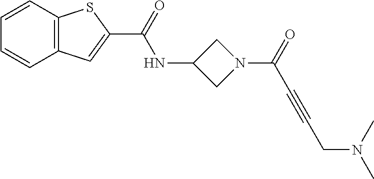 [SMILES: CN(C)CC#CC(=O)N1CC(NC(=O)c2cc3ccccc3s2)C1] | N-(1-(4- (dimethylamino)but-2- ynoyl)azetidin-3- yl)benzo[b]thiophene-2- carboxamide |
| 225 | XS224- 156 | 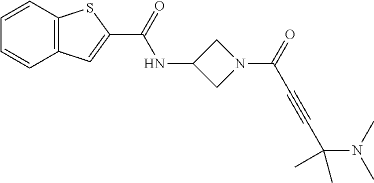 [SMILES: CN(C)C(C)(C)C#CC(=O)N1CC(NC(=O)c2cc3ccccc3s2)C1] | N-(1-(4-(dimethylamino)-4- methylpent-2- ynoyl)azetidin-3- yl)benzo[b]thiophene-2- carboxamide |
| 226 | XS224- 157 | 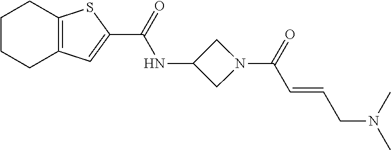 [SMILES: CN(C)C/C=C/C(=O)N1CC(NC(=O)c2cc3c(s2)CCCC3)C1] | (E)-N-(1-(4- (dimethylamino)but-2- enoyl)azetidin-3-yl)-4,5,6,7- tetrahydrobenzo[b] thiophene-2-carboxamide |
| 227 | XS224- 158 | 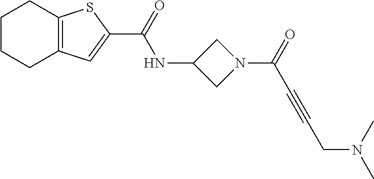 [SMILES: CN(C)CC#CC(=O)N1CC(NC(=O)c2cc3c(s2)CCCC3)C1] | N-(1-(4- (dimethylamino)but-2- ynoyl)azetidin-3-yl)-4,5,6,7- tetrahydrobenzo[b] thiophene-2-carboxamide |
| 228 | XS224- 159 | 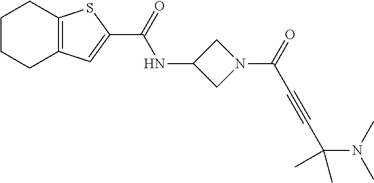 [SMILES: CN(C)C(C)(C)C#CC(=O)N1CC(NC(=O)c2cc3c(s2)CCCC3)C1] | N-(1-(4-(dimethylamino)-4- methylpent-2- ynoyl)azctidin-3-yl)-4,5,6,7- tetrahydrobenzo[b] thiophene-2-carboxamide |

Examples of AMPK based bivalent compounds are set forth in table 2 below.

TABLE 2

| | Note | |
|---|---|---|
| Examples | ID | Structure |
| 229 | XF137-81 | 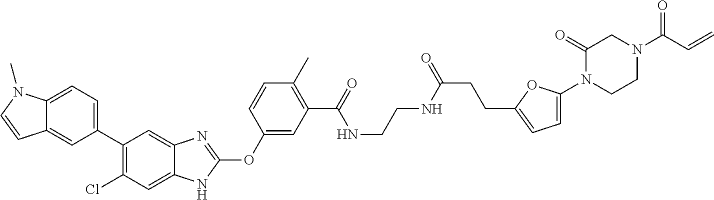 [SMILES: C=CC(=O)N1CCN(c2ccc(CCC(=O)NCCNC(=O)c3cc(Oc4nc5cc(-c6ccc7c(ccn7C)c6)c(Cl)cc5[nH]4)ccc3C)o2)C(=O)C1] |
| 230 | XF137-82 | 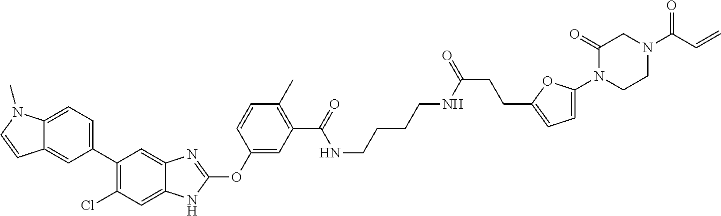 [SMILES: C=CC(=O)N1CCN(c2ccc(CCC(=O)NCCCCNC(=O)c3cc(Oc4nc5cc(-c6ccc7c(ccn7C)c6)c(Cl)cc5[nH]4)ccc3C)o2)C(=O)C1] |
| 231 | XF137-83 | 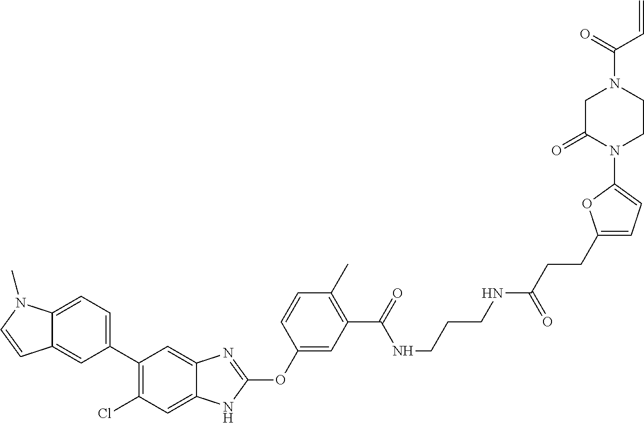 [SMILES: C=CC(=O)N1CCN(c2ccc(CCC(=O)NCCCNC(=O)c3cc(Oc4nc5cc(-c6ccc7c(ccn7C)c6)c(Cl)cc5[nH]4)ccc3C)o2)C(=O)C1] |
| 232 | XF137-84 | 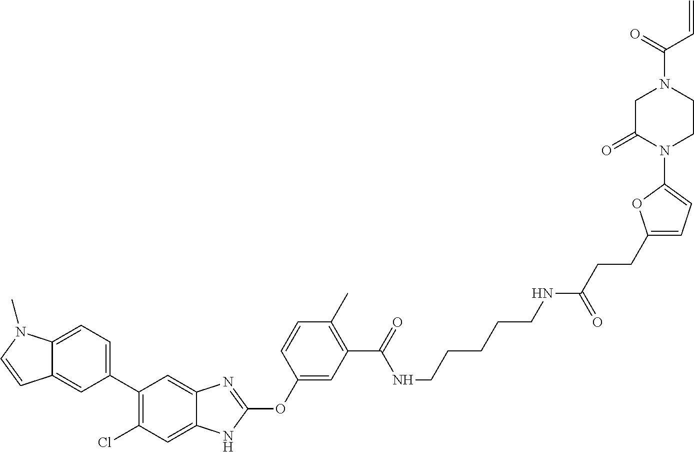 [SMILES: C=CC(=O)N1CCN(c2ccc(CCC(=O)NCCCCCNC(=O)c3cc(Oc4nc5cc(-c6ccc7c(ccn7C)c6)c(Cl)cc5[nH]4)ccc3C)o2)C(=O)C1] |
| 233 | XF137-85 | 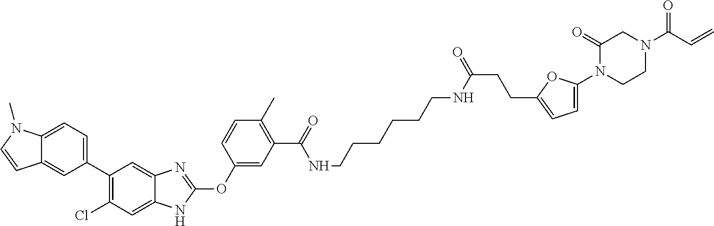 [SMILES: C=CC(=O)N1CCN(c2ccc(CCC(=O)NCCCCCCNC(=O)c3cc(Oc4nc5cc(-c6ccc7c(ccn7C)c6)c(Cl)cc5[nH]4)ccc3C)o2)C(=O)C1] |
| 234 | XF137-86 | 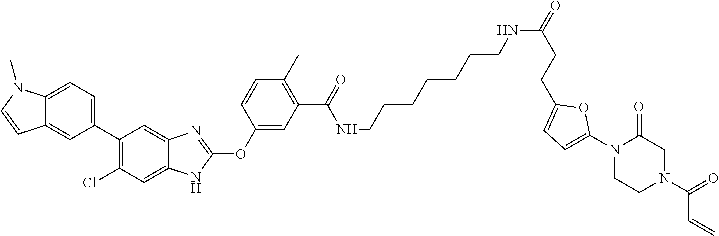 [SMILES: C=CC(=O)N1CCN(c2ccc(CCC(=O)NCCCCCCCNC(=O)c3cc(Oc4nc5cc(-c6ccc7c(ccn7C)c6)c(Cl)cc5[nH]4)ccc3C)o2)C(=O)C1] |
| 235 | XF137-87 | 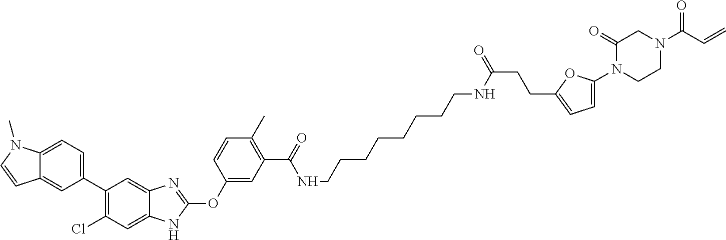 [SMILES: C=CC(=O)N1CCN(c2ccc(CCC(=O)NCCCCCCCCNC(=O)c3cc(Oc4nc5cc(-c6ccc7c(ccn7C)c6)c(Cl)cc5[nH]4)ccc3C)o2)C(=O)C1] |
| 236 | XF137-88 | 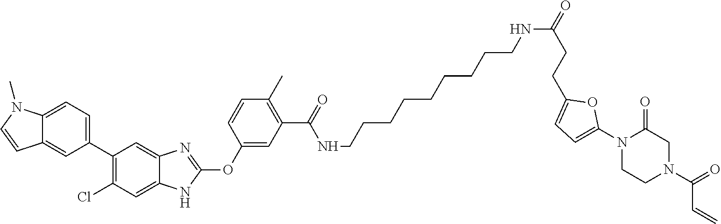 [SMILES: C=CC(=O)N1CCN(c2ccc(CCC(=O)NCCCCCCCCCNC(=O)c3cc(Oc4nc5cc(-c6ccc7c(ccn7C)c6)c(Cl)cc5[nH]4)ccc3C)o2)C(=O)C1] |
| 237 | XF137-89 | 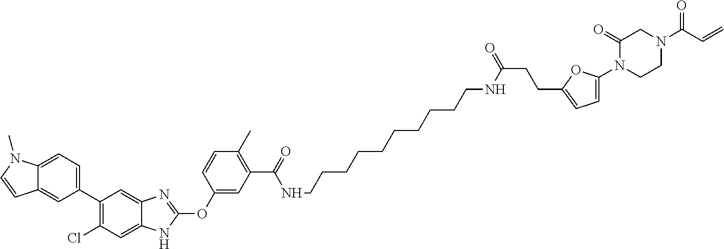 [SMILES: C=CC(=O)N1CCN(c2ccc(CCC(=O)NCCCCCCCCCCNC(=O)c3cc(Oc4nc5cc(-c6ccc7c(ccn7C)c6)c(Cl)cc5[nH]4)ccc3C)o2)C(=O)C1] |
| 238 | XF137-90 | 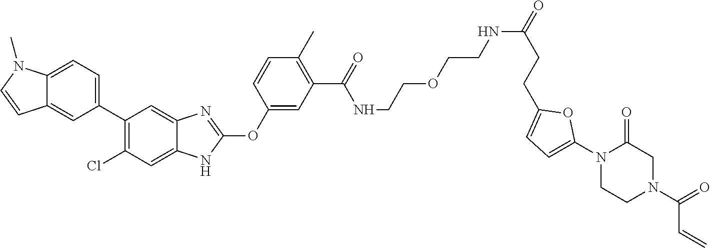 [SMILES: C=CC(=O)N1CCN(c2ccc(CCC(=O)NCCOCCNC(=O)c3cc(Oc4nc5cc(-c6ccc7c(ccn7C)c6)c(Cl)cc5[nH]4)ccc3C)o2)C(=O)C1] |
| 239 | XF137-91 | 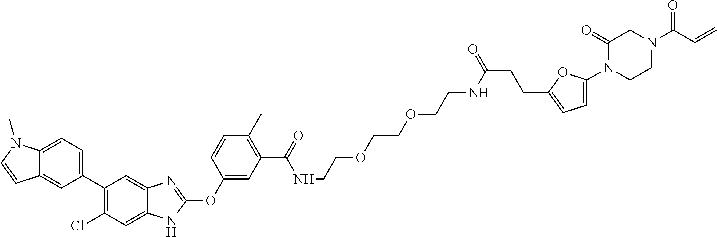 [SMILES: C=CC(=O)N1CCN(c2ccc(CCC(=O)NCCOCCOCCNC(=O)c3cc(Oc4nc5cc(-c6ccc7c(ccn7C)c6)c(Cl)cc5[nH]4)ccc3C)o2)C(=O)C1] |
| 240 | XF137-92 | 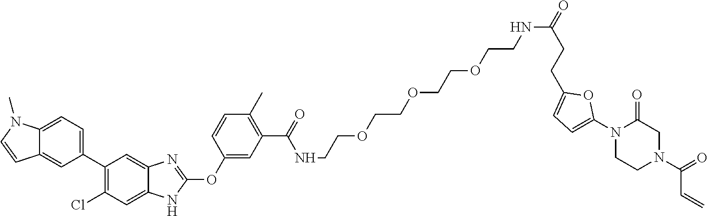 [SMILES: C=CC(=O)N1CCN(c2ccc(CCC(=O)NCCOCCOCCOCCNC(=O)c3cc(Oc4nc5cc(-c6ccc7c(ccn7C)c6)c(Cl)cc5[nH]4)ccc3C)o2)C(=O)C1] |
| 241 | XF137-93 | 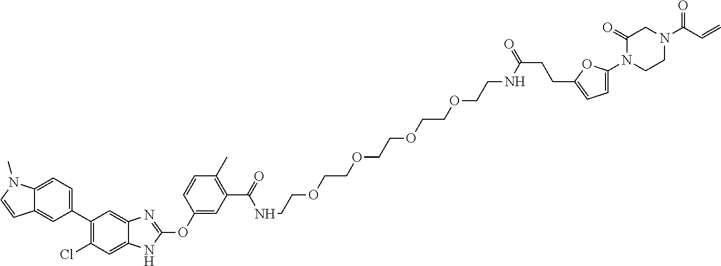 [SMILES: C=CC(=O)N1CCN(c2ccc(CCC(=O)NCCOCCOCCOCCOCCNC(=O)c3cc(Oc4nc5cc(-c6ccc7c(ccn7C)c6)c(Cl)cc5[nH]4)ccc3C)o2)C(=O)C1] |
| 242 | XF137-94 | 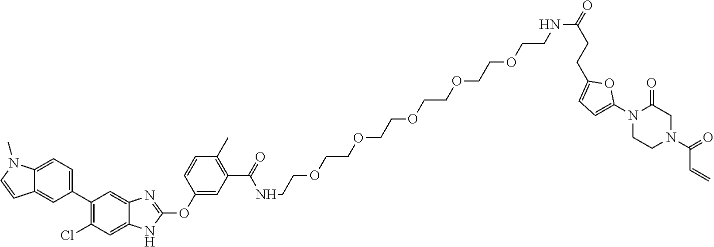 [SMILES: C=CC(=O)N1CCN(c2ccc(CCC(=O)NCCOCCOCCOCCOCCOCCNC(=O)c3cc(Oc4nc5cc(-c6ccc7c(ccn7C)c6)c(Cl)cc5[nH]4)ccc3C)o2)C(=O)C1] |
| 243 | QC179-047 | 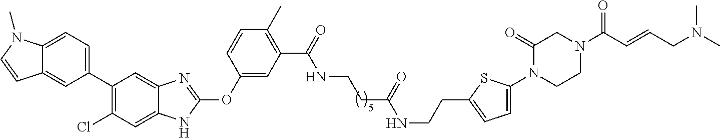 |
| 244 | QC179-048 | 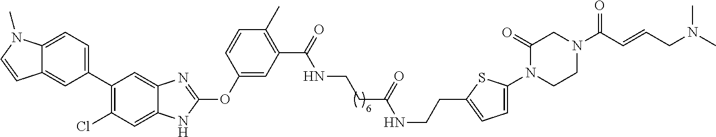 |
| 245 | QC179-049 | 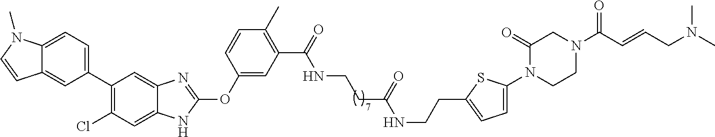 |
| 246 | QC179-050 | 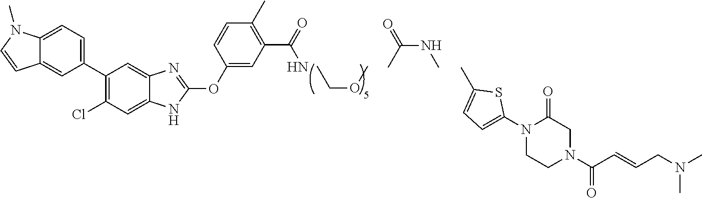 |
| 247 | QC179-051 | 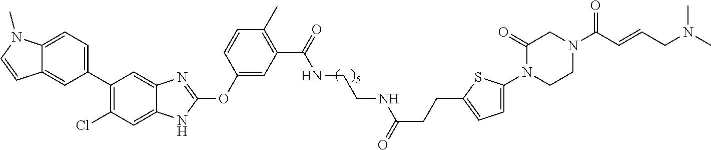 |
| 248 | QC179-052 | 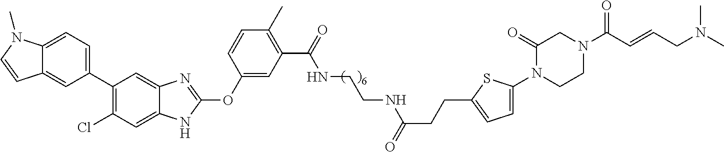 |
| 249 | QC179-053 | 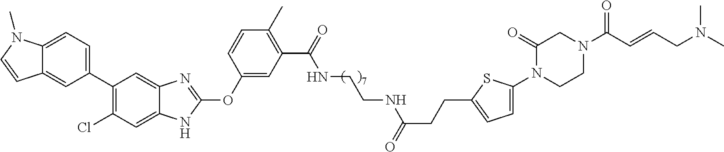 |
| 250 | QC179-054 | 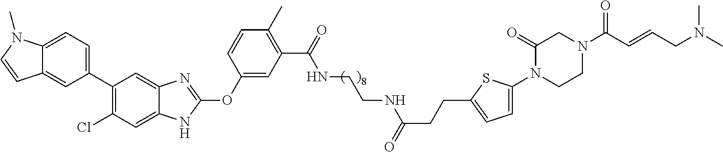 |
| 251 | QC179-055 | 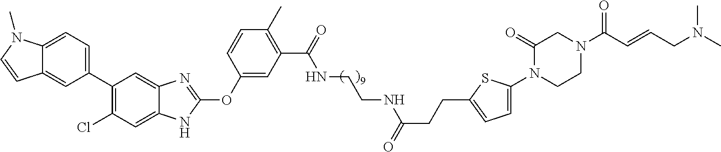 |
| | Examples | Chemical Name |
| | 229 | N-(2-(3-(5-(4-acryloyl-2-oxopiperazin-1-yl)furan-2-yl)propanamido)ethyl)-5-((6-chloro-5-(1- |
| | | methyl-1H-indol-5-yl)-1H-benzo[d]imidazol-2-yl)oxy)-2-methylbenzamide |
| | 230 | N-(4-(3-(5-(4-acryloyl-2-oxopiperazin-1-yl)furan-2-yl)propanamido)butyl)-5-((6-chloro-5-(1- |
| | | methyl-1H-indol-5-yl)-1H-benzo[d]imidazol-2-yl)oxy)-2-methylbenzamide |
| | 231 | N-(3-(3-(5-(4-acryloyl-2-oxopiperazin-1-yl)furan-2-yl)propanamido)propyl)-5-((6-chloro-5-(1- |
| | | methyl-1H-indol-5-yl)-1H-benzo[d]imidazol-2-yl)oxy)-2-methylbenzamide |
| | 232 | N-(5-(3-(5-(4-acryloyl-2-oxopiperazin-1-yl)furan-2-yl)propanamido)pentyl)-5-((6-chloro-5-(1- |
| | | methyl-1H-indol-5-yl)-1H-benzo[d]imidazol-2-yl)oxy)-2-methylbenzamide |
| | 233 | N-(6-(3-(5-(4-acryloyl-2-oxopiperazin-1-yl)furan-2-yl)propanamido)hexyl)-5-((6-chloro-5-(1- |
| | | methyl-1H-indol-5-yl)-1H-benzo[d]imidazol-2-yl)oxy)-2-methylbenzamide |
| | 234 | N-(7-(3-(5-(4-acryloyl-2-oxopiperazin-1-yl)furan-2-yl)propanamido)heptyl)-5-((6-chloro-5-(1- |
| | | methyl-1H-indol-5-yl)-1H-benzo[d]imidazol-2-yl)oxy)-2-methylbenzamide |
| | 235 | N-(8-(3-(5-(4-acryloyl-2-oxopiperazin-1-yl)furan-2-yl)propanamido)octyl)-5-((6-chloro-5-(1- |
| | | methyl-1H-indol-5-yl)-1H-benzo[d]imidazol-2-yl)oxy)-2-methylbenzamide |
| | 236 | N-(9-(3-(5-(4-acryloyl-2-oxopiperazin-1-yl)furan-2-yl)propanamido)nonyl)-5-((6-chloro-5-(1- |
| | | methyl-1H-indol-5-yl)-1H-benzo[d]imidazol-2-yl)oxy)-2-methylbenzamide |
| | 237 | N-(10-(3-(5-(4-acryloyl-2-oxopiperazin-1-yl)furan-2-yl)propanamido)decyl)-5-((6-chloro-5-(1- |
| | | methyl-1H-indol-5-yl)-1H-benzo[d]imidazol-2-yl)oxy)-2-methylbenzamide |
| | 238 | N-(2-(2-(3-(5-(4-acryloyl-2-oxopiperazin-1-yl)furan-2-yl)propanamido)ethoxy)ethyl)-5-((6-chloro-5-(1- |
| | | methyl-1H-indol-5-yl)-1H-benzo[d]imidazol-2-yl)oxy)-2-methylbenzamide |
| | 239 | N-(2-(2-(2-(3-(5-(4-acryloyl-2-oxopiperazin-1-yl)furan-2-yl)propanamido)ethoxy)ethoxy)ethyl)-5-((6-chloro-5-(1- |
| | | methyl-1H-indol-5-yl)-1H-benzo[d]imidazol-2-yl)oxy)-2-methylbenzamide |
| | 240 | N-(15-(5-(4-acryloyl-2-oxopiperazin-1-yl)furan-2-yl)-13-oxo-3,6,9-trioxa-12-azapentadecyl)-5-((6-chloro-5-(1- |
| | | methyl-1H-indol-5-yl)-1H-benzo[d]imidazol-2-yl)oxy)-2-methylbenzamide |
| | 241 | N-(18-(5-(4-acryloyl-2-oxopiperazin-1-yl)furan-2-yl)-16-oxo-3,6,9,12-tetraoxa-15-azaoctadecyl)-5-((6- |
| | | chloro-5-(1-methyl-1H-indol-5-yl)-1H-benzo[d]imidazol-2-yl)oxy)-2-methylbenzamide |
| | 242 | N-(21-(5-(4-acryloyl-2-oxopiperazin-1-yl)furan-2-yl)-19-oxo-3,6,9,12,15-pentaoxa-18-azahenicosyl)-5- |
| | | ((6-chloro-5-(1-methyl-1H-indol-5-yl)-1H-benzo[d]imidazol-2-yl)oxy)-2-methylbenzamide |
| | 243 | (E)-5-((6-chloro-5-(1-methyl-1H-indol-5-yl)-1H-benzo[d]imidazol-2-yl)oxy)-N-(7-((2-(5-(4-(4- |
| | | (dimethylamino)but-2-enoyl)-2-oxopiperazin-1-yl)thiophen-2-yl)ethyl)amino)-7-oxoheptyl)-2-methylbenzamide |
| | 244 | (E)-5-((6-chloro-5-(1-methyl-1H-indol-5-yl)-1H-benzo[d]imidazol-2-yl)oxy)-N-(8-((2-(5-(4-(4- |
| | | (dimethylamino)but-2-enoyl-2-oxopiperazin-1-yl)thiophen-2-yl)ethyl)amino)-8-oxooctyl)-2-methylbenzamide |
| | 245 | (E)-5-((6-chloro-5-(1-methyl-1H-indol-5-yl)-1H-benzo[d]imidazol-2-yl)oxy)-N-(9-((2-(5-(4-(4- |
| | | (dimethylamino)but-2-enoyl)-2-oxopiperazin-1-yl)thiophen-2-yl)ethyl)amino)-9-oxononyl)-2-methylbenzamide |
| | 246 | (E)-1-(5-((6-chloro-5-(1-methyl-1H-indol-5-yl)-1H-benzo[d]imidazol-2-yl)oxy)-2-methylbenzamido)-N- |
| | | (2-(5-(4-(4-(dimethylamino)but-2-enoyl)-2-oxopiperazin-1-yl)thiophen-2-yl)ethyl)-3,6,9,12,15- |
| | | pentaoxaoctadecan-18-amide |
| | 247 | (E)-N-(2-((5-((6-chloro-5-(1-methyl-1H-indol-5-yl)-1H-benzo[d]imidazol-2-yl)oxy)-2-methylbenzoyl) |
| | | tetramethyl-1⁷-azaneyl)ethyl)-3-(5-(4-(4-(dimethylamino)but-2-enoyl)-2-oxopiperazin-1-yl)thiophen-2- |
| | | yl)propanamide |
| | 248 | (E)-N-(2-((5-((6-chloro-5-(1-methyl-1H-indol-5-yl)-1H-benzo[d]imidazol-2-yl)oxy)-2-methylbenzoyl) |
| | | pentamethyl-1⁸-azaneyl)ethyl)-3-(5-(4-(4-(dimethylamino)but-2-enoyl)-2-oxopiperazin-1-yl)thiophen- |
| | | 2-yl)propanamide |
| | 249 | (E)-N-(2-((5-((6-chloro-5-(1-methyl-1H-indol-5-yl)-1H-benzo[d]imidazol-2-yl)oxy)-2-methylbenzoyl) |
| | | hexamethyl-1⁹-azaneyl)ethyl)-3-(5-(4-(4-(dimethylamino)but-2-enoyl)-2-oxopiperazin-1-yl)thiophen- |
| | | 2-yl)propanamide |
| | 250 | (E)-N-(2-((5-((6-chloro-5-(1-methyl-1H-indol-5-yl)-1H-benzo[d]imidazol-2-yl)oxy)-2-methylbenzoyl) |
| | | heptamethyl-1¹⁰-azaneyl)ethyl)-3-(5-(4-(4-(dimethylamino)but-2-enoyl)-2-oxopiperazin-1-yl)thiophen- |
| | | 2-yl)propanamide |
| | 251 | (E)-N-(2-((5-((6-chloro-5-(1-methyl-1H-indol-5-yl)-1H-benzo[d]imidazol-2-yl)oxy)-2-methylbenzoyl) |
| | | octamethyl-1¹¹-azaneyl)ethyl)-3-(5-(4-(4-(dimethylamino)but-2-enoyl)-2-oxopiperazin-1-yl)thiophen- |
| | | 2-yl)propanamide |

Examples of cGAS based bivalent compounds are set forth in table 3 below.

TABLE 3

| Example | Cpd | |
|---|---|---|
| No. | ID | Structure |
| 252 | ZD178- 22 (H1) | 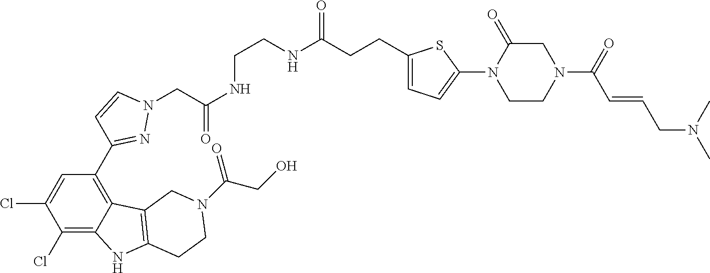 [SMILES: CN(C)C/C=C/C(=O)N1CCN(c2ccc(CCC(=O)NCCNC(=O)Cn3ccc(-c4cc(Cl)c(Cl)c5[nH]c6c(c45)CN(C(=O)CO)CC6)n3)s2)C(=O)C1] |
| 253 | ZD178- 23 (H2) | 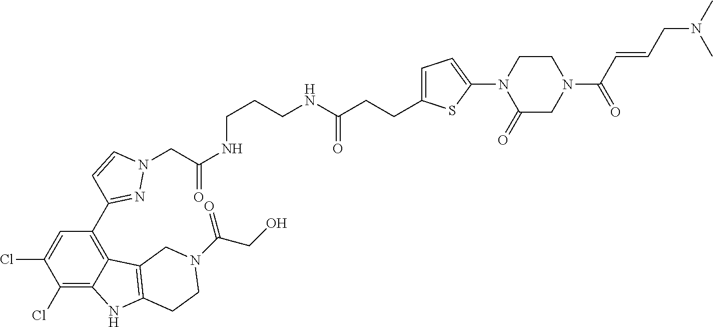 [SMILES: CN(C)C/C=C/C(=O)N1CCN(c2ccc(CCC(=O)NCCCNC(=O)Cn3ccc(-c4cc(Cl)c(Cl)c5[nH]c6c(c45)CN(C(=O)CO)CC6)n3)s2)C(=O)C1] |
| 254 | ZD178- 24 (H3) | 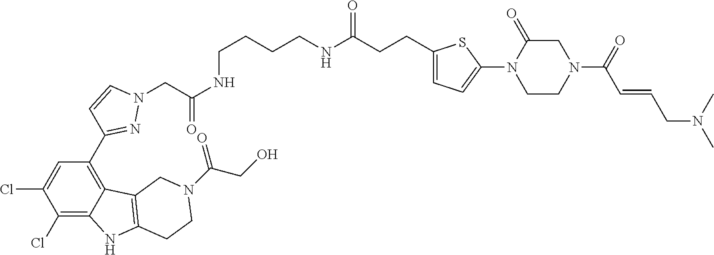 [SMILES: CN(C)C/C=C/C(=O)N1CCN(c2ccc(CCC(=O)NCCCCNC(=O)Cn3ccc(-c4cc(Cl)c(Cl)c5[nH]c6c(c45)CN(C(=O)CO)CC6)n3)s2)C(=O)C1] |
| 255 | ZD178- 25 (H4) | 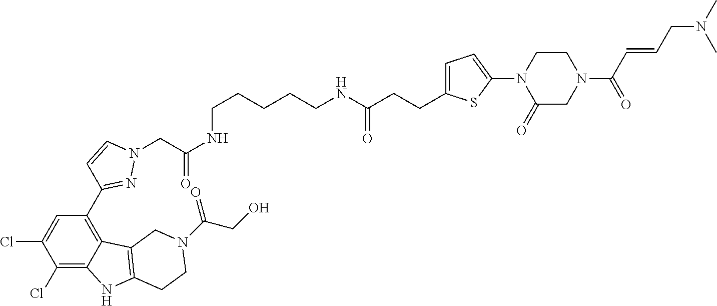 [SMILES: CN(C)C/C=C/C(=O)N1CCN(c2ccc(CCC(=O)NCCCCCNC(=O)Cn3ccc(-c4cc(Cl)c(Cl)c5[nH]c6c(c45)CN(C(=O)CO)CC6)n3)s2)C(=O)C1] |
| 256 | ZD178- 26 (H5) | 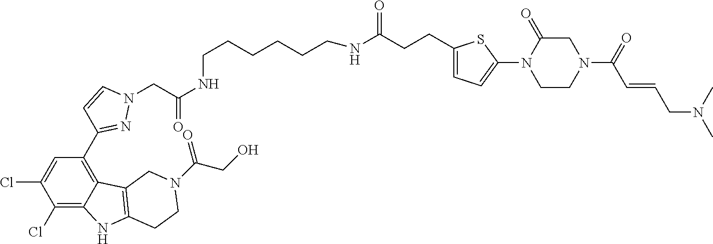 [SMILES: CN(C)C/C=C/C(=O)N1CCN(c2ccc(CCC(=O)NCCCCCCNC(=O)Cn3ccc(-c4cc(Cl)c(Cl)c5[nH]c6c(c45)CN(C(=O)CO)CC6)n3)s2)C(=O)C1] |
| 257 | ZD178- 27 (H6) | 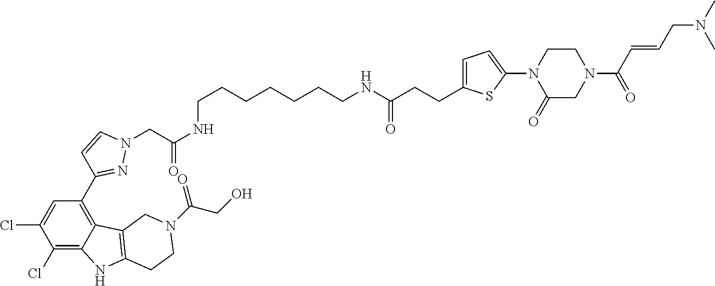 [SMILES: CN(C)C/C=C/C(=O)N1CCN(c2ccc(CCC(=O)NCCCCCCCNC(=O)Cn3ccc(-c4cc(Cl)c(Cl)c5[nH]c6c(c45)CN(C(=O)CO)CC6)n3)s2)C(=O)C1] |
| 258 | ZD178- 28 (H7) | 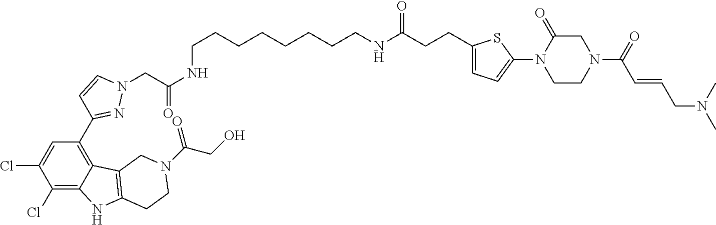 [SMILES: CN(C)C/C=C/C(=O)N1CCN(c2ccc(CCC(=O)NCCCCCCCCNC(=O)Cn3ccc(-c4cc(Cl)c(Cl)c5[nH]c6c(c45)CN(C(=O)CO)CC6)n3)s2)C(=O)C1] |
| 259 | ZD178- 29 (MS7829)/ (H8) | 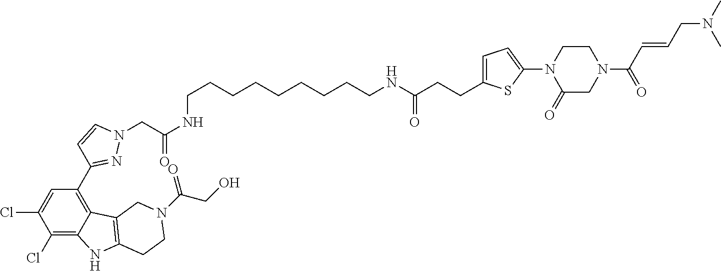 [SMILES: CN(C)C/C=C/C(=O)N1CCN(c2ccc(CCC(=O)NCCCCCCCCCNC(=O)Cn3ccc(-c4cc(Cl)c(Cl)c5[nH]c6c(c45)CN(C(=O)CO)CC6)n3)s2)C(=O)C1] |
| 260 | ZD178- 30 (H9) | 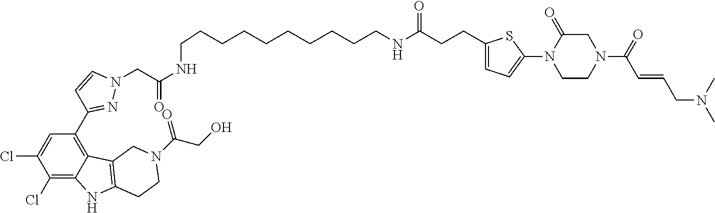 [SMILES: CN(C)C/C=C/C(=O)N1CCN(c2ccc(CCC(=O)NCCCCCCCCCCNC(=O)Cn3ccc(-c4cc(Cl)c(Cl)c5[nH]c6c(c45)CN(C(=O)CO)CC6)n3)s2)C(=O)C1] |
| 261 | ZD178- 31 (H10) | 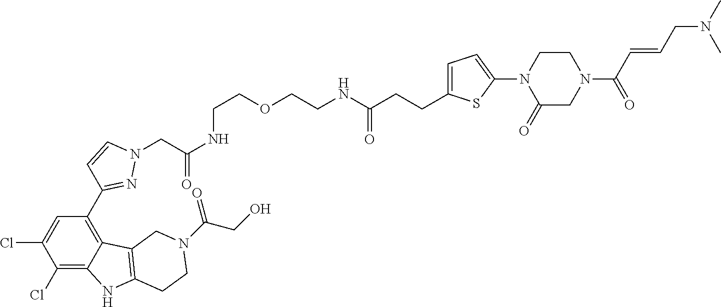 [SMILES: CN(C)C/C=C/C(=O)N1CCN(c2ccc(CCC(=O)NCCOCCNC(=O)Cn3ccc(-c4cc(Cl)c(Cl)c5[nH]c6c(c45)CN(C(=O)CO)CC6)n3)s2)C(=O)C1] |
| 262 | ZD178- 32 (H11) | 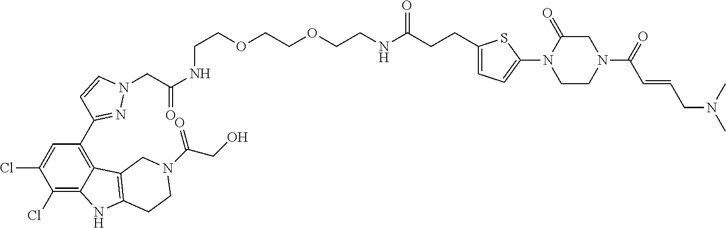 [SMILES: CN(C)C/C=C/C(=O)N1CCN(c2ccc(CCC(=O)NCCOCCOCCNC(=O)Cn3ccc(-c4cc(Cl)c(Cl)c5[nH]c6c(c45)CN(C(=O)CO)CC6)n3)s2)C(=O)C1] |
| 263 | ZD178- 33 (H12) | 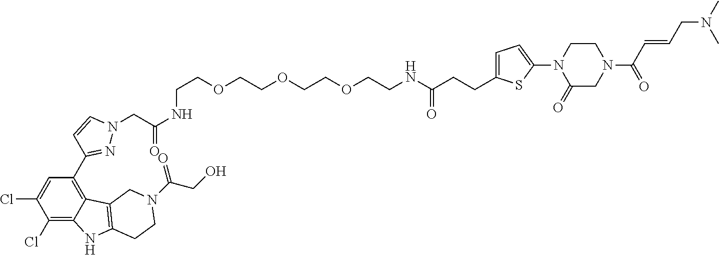 [SMILES: CN(C)C/C=C/C(=O)N1CCN(c2ccc(CCC(=O)NCCOCCOCCOCCNC(=O)Cn3ccc(-c4cc(Cl)c(Cl)c5[nH]c6c(c45)CN(C(=O)CO)CC6)n3)s2)C(=O)C1] |
| 264 | ZD178- 34 (H13) | 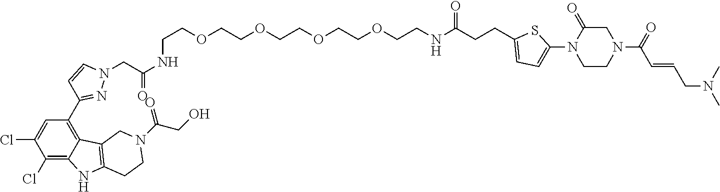 [SMILES: CN(C)C/C=C/C(=O)N1CCN(c2ccc(CCC(=O)NCCOCCOCCOCCOCCNC(=O)Cn3ccc(-c4cc(Cl)c(Cl)c5[nH]c6c(c45)CN(C(=O)CO)CC6)n3)s2)C(=O)C1] |
| 265 | ZD178- 35 (H14) | 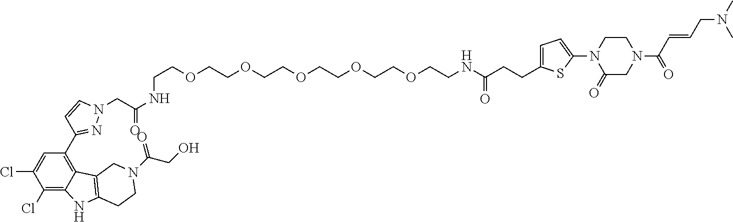 [SMILES: CN(C)C/C=C/C(=O)N1CCN(c2ccc(CCC(=O)NCCOCCOCCOCCOCCOCCNC(=O)Cn3ccc(-c4cc(Cl)c(Cl)c5[nH]c6c(c45)CN(C(=O)CO)CC6)n3)s2)C(=O)C1] |
| 266 | ZD178- 58-1 (Z1) | 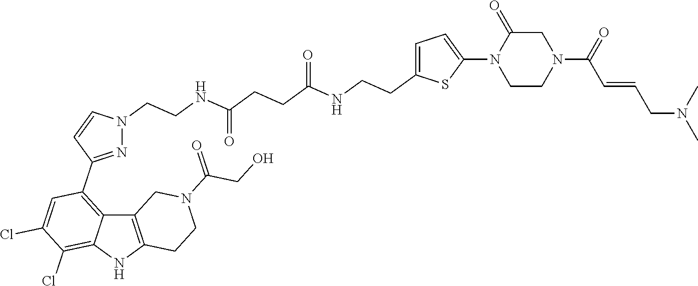 [SMILES: CN(C)C/C=C/C(=O)N1CCN(c2ccc(CCNC(=O)CCC(=O)NCCn3ccc(-c4cc(Cl)c(Cl)c5[nH]c6c(c45)CN(C(=O)CO)CC6)n3)s2)C(=O)C1] |
| 267 | ZD178- 58-2 (Z2) | 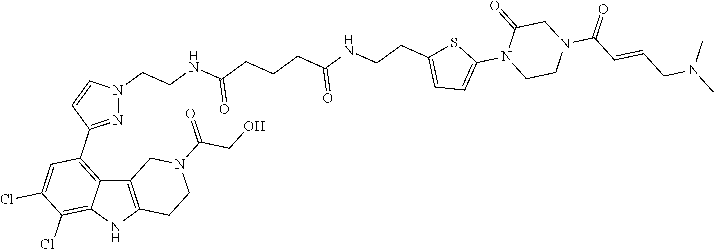 [SMILES: CN(C)C/C=C/C(=O)N1CCN(c2ccc(CCNC(=O)CCCC(=O)NCCn3ccc(-c4cc(Cl)c(Cl)c5[nH]c6c(c45)CN(C(=O)CO)CC6)n3)s2)C(=O)C1] |
| 268 | ZD178- 58-3 (Z3) | 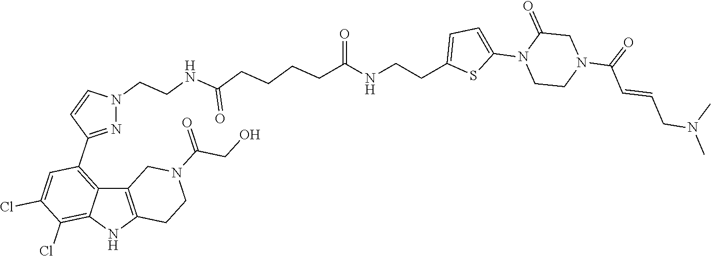 [SMILES: CN(C)C/C=C/C(=O)N1CCN(c2ccc(CCNC(=O)CCCCC(=O)NCCn3ccc(-c4cc(Cl)c(Cl)c5[nH]c6c(c45)CN(C(=O)CO)CC6)n3)s2)C(=O)C1] |
| 269 | ZD178- 58-4 (Z4) | 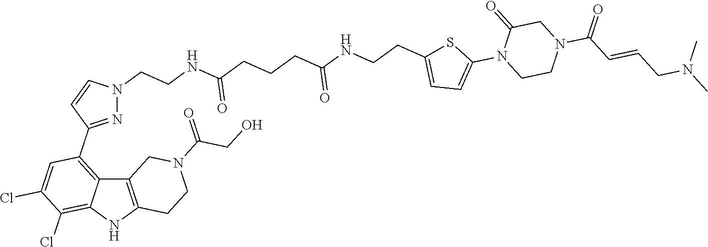 [SMILES: CN(C)C/C=C/C(=O)N1CCN(c2ccc(CCNC(=O)CCCC(=O)NCCn3ccc(-c4cc(Cl)c(Cl)c5[nH]c6c(c45)CN(C(=O)CO)CC6)n3)s2)C(=O)C1] |
| 270 | ZD178- 58-5 (Z5) | 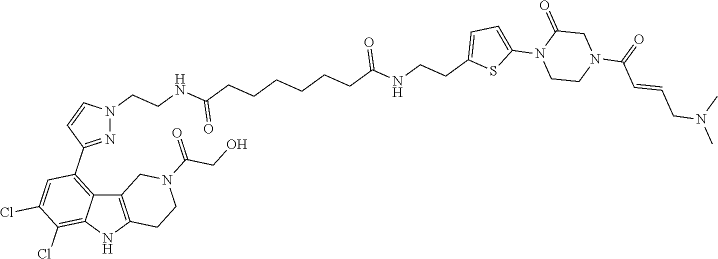 [SMILES: CN(C)C/C=C/C(=O)N1CCN(c2ccc(CCNC(=O)CCCCCCC(=O)NCCn3ccc(-c4cc(Cl)c(Cl)c5[nH]c6c(c45)CN(C(=O)CO)CC6)n3)s2)C(=O)C1] |
| 271 | ZD178- 58-6 (Z6) | 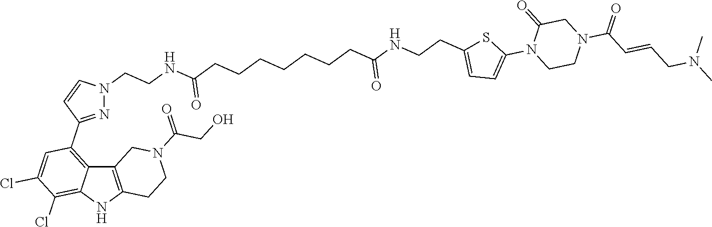 [SMILES: CN(C)C/C=C/C(=O)N1CCN(c2ccc(CCNC(=O)CCCCCCCC(=O)NCCn3ccc(-c4cc(Cl)c(Cl)c5[nH]c6c(c45)CN(C(=O)CO)CC6)n3)s2)C(=O)C1] |
| 272 | ZD178- 58-7 (Z7) | 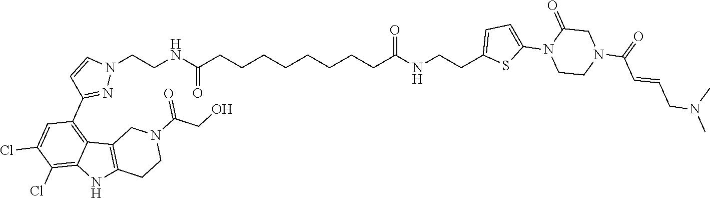 [SMILES: CN(C)C/C=C/C(=O)N1CCN(c2ccc(CCNC(=O)CCCCCCCCC(=O)NCCn3ccc(-c4cc(Cl)c(Cl)c5[nH]c6c(c45)CN(C(=O)CO)CC6)n3)s2)C(=O)C1] |
| 273 | ZD178- 58-8 (MS8588)/ (Z8) | 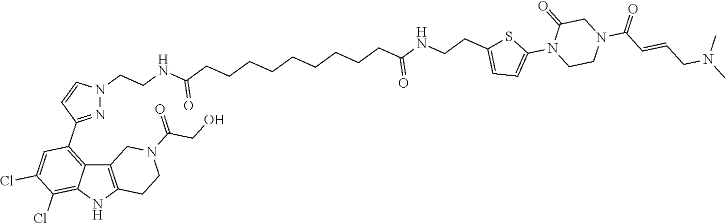 [SMILES: CN(C)C/C=C/C(=O)N1CCN(c2ccc(CCNC(=O)CCCCCCCCCC(=O)NCCn3ccc(-c4cc(Cl)c(Cl)c5[nH]c6c(c45)CN(C(=O)CO)CC6)n3)s2)C(=O)C1] |
| 274 | ZD178- 58-9 (Z9) | 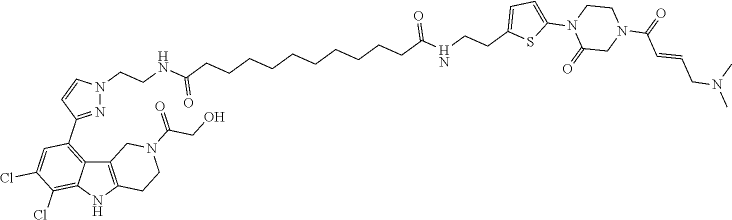 [SMILES: CN(C)C/C=C/C(=O)N1CCN(c2ccc(CCNC(=O)CCCCCCCCCCC(=O)NCCn3ccc(-c4cc(Cl)c(Cl)c5[nH]c6c(c45)CN(C(=O)CO)CC6)n3)s2)C(=O)C1] |
| 275 | ZD178- 58-10 (Z10) | 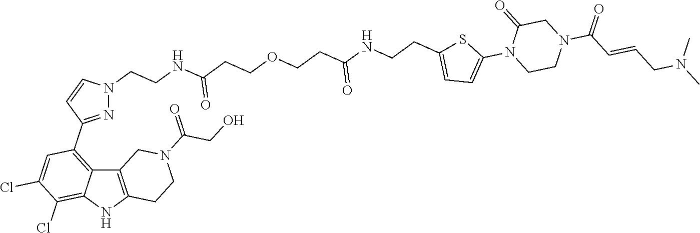 [SMILES: CN(C)C/C=C/C(=O)N1CCN(c2ccc(CCNC(=O)CCOCCC(=O)NCCn3ccc(-c4cc(Cl)c(Cl)c5[nH]c6c(c45)CN(C(=O)CO)CC6)n3)s2)C(=O)C1] |
| 276 | ZD178- 58-11 (Z11) | 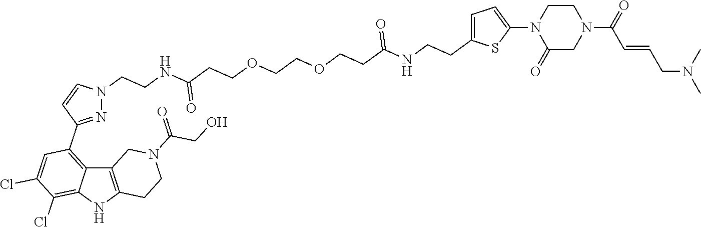 [SMILES: CN(C)C/C=C/C(=O)N1CCN(c2ccc(CCNC(=O)CCOCCOCCC(=O)NCCn3ccc(-c4cc(Cl)c(Cl)c5[nH]c6c(c45)CN(C(=O)CO)CC6)n3)s2)C(=O)C1] |
| 277 | ZD178- 58-12 (Z12) | 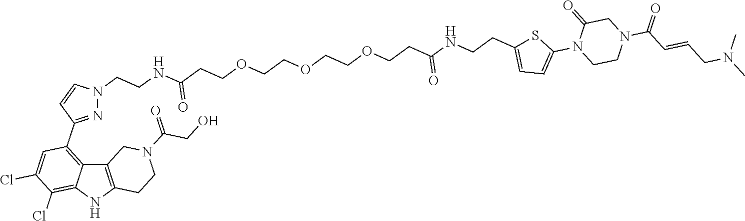 [SMILES: CN(C)C/C=C/C(=O)N1CCN(c2ccc(CCNC(=O)CCOCCOCCOCCC(=O)NCCn3ccc(-c4cc(Cl)c(Cl)c5[nH]c6c(c45)CN(C(=O)CO)CC6)n3)s2)C(=O)C1] |
| 278 | ZD178- 58-13 (Z13) | 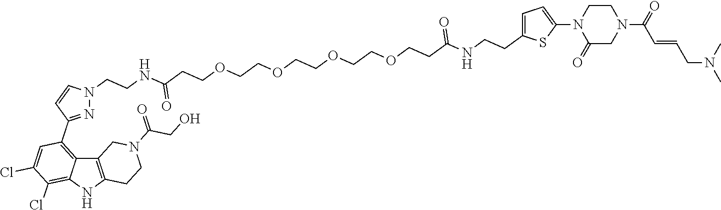 [SMILES: CN(C)C/C=C/C(=O)N1CCN(c2ccc(CCNC(=O)CCOCCOCCOCCOCCC(=O)NCCn3ccc(-c4cc(Cl)c(Cl)c5[nH]c6c(c45)CN(C(=O)CO)CC6)n3)s2)C(=O)C1] |
| 279 | ZD178- 58-14 (Z14) | 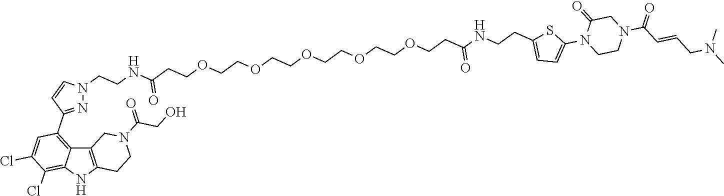 [SMILES: CN(C)C/C=C/C(=O)N1CCN(c2ccc(CCNC(=O)CCOCCOCCOCCOCCOCCC(=O)NCCn3ccc(-c4cc(Cl)c(Cl)c5[nH]c6c(c45)CN(C(=O)CO)CC6)n3)s2)C(=O)C1] |
| 280 | ZD178- 63-1 (M1) | 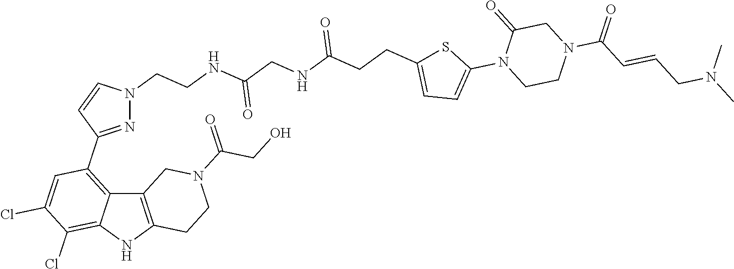 [SMILES: CN(C)C/C=C/C(=O)N1CCN(c2ccc(CCC(=O)NCC(=O)NCCn3ccc(-c4cc(Cl)c(Cl)c5[nH]c6c(c45)CN(C(=O)CO)CC6)n3)s2)C(=O)C1] |
| 281 | ZD178- 63-2 (M2) | 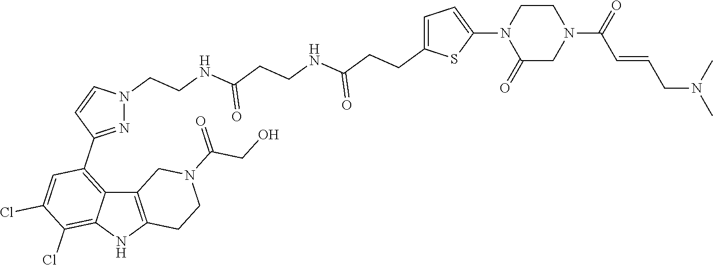 [SMILES: CN(C)C/C=C/C(=O)N1CCN(c2ccc(CCC(=O)NCCC(=O)NCCn3ccc(-c4cc(Cl)c(Cl)c5[nH]c6c(c45)CN(C(=O)CO)CC6)n3)s2)C(=O)C1] |
| 282 | ZD178- 63-3 (M3) | 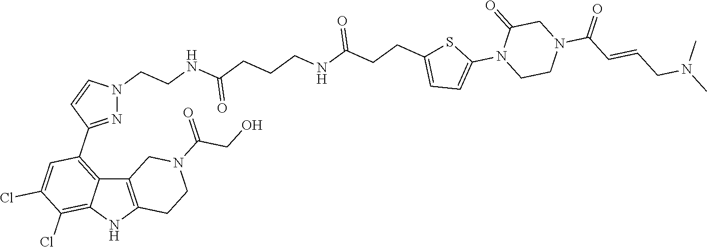 [SMILES: CN(C)C/C=C/C(=O)N1CCN(c2ccc(CCC(=O)NCCCC(=O)NCCn3ccc(-c4cc(Cl)c(Cl)c5[nH]c6c(c45)CN(C(=O)CO)CC6)n3)s2)C(=O)C1] |
| 283 | ZD178- 63-4 (M4) | 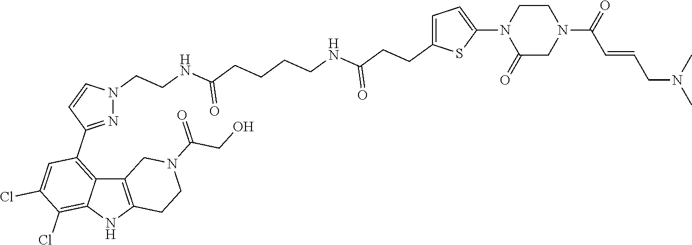 [SMILES: CN(C)C/C=C/C(=O)N1CCN(c2ccc(CCC(=O)NCCCCC(=O)NCCn3ccc(-c4cc(Cl)c(Cl)c5[nH]c6c(c45)CN(C(=O)CO)CC6)n3)s2)C(=O)C1] |
| 284 | ZD178- 63-5 (M5) | 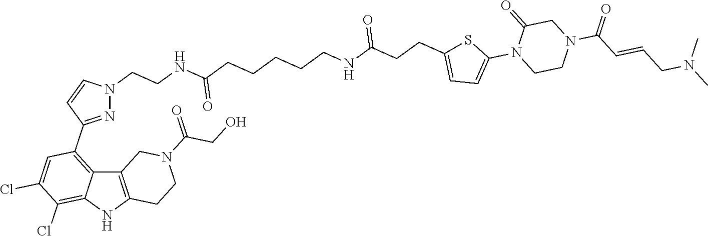 [SMILES: CN(C)C/C=C/C(=O)N1CCN(c2ccc(CCC(=O)NCCCCCC(=O)NCCn3ccc(-c4cc(Cl)c(Cl)c5[nH]c6c(c45)CN(C(=O)CO)CC6)n3)s2)C(=O)C1] |
| 285 | ZD178- 63-6 (M6) | 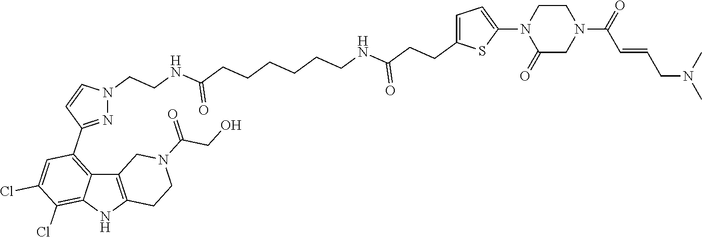 [SMILES: CN(C)C/C=C/C(=O)N1CCN(c2ccc(CCC(=O)NCCCCCCC(=O)NCCn3ccc(-c4cc(Cl)c(Cl)c5[nH]c6c(c45)CN(C(=O)CO)CC6)n3)s2)C(=O)C1] |
| 286 | ZD178- 63-7 (M7) | 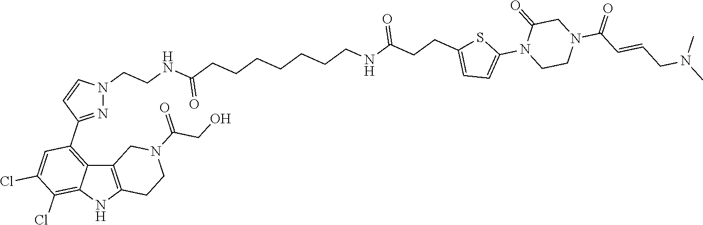 [SMILES: CN(C)C/C=C/C(=O)N1CCN(c2ccc(CCC(=O)NCCCCCCCC(=O)NCCn3ccc(-c4cc(Cl)c(Cl)c5[nH]c6c(c45)CN(C(=O)CO)CC6)n3)s2)C(=O)C1] |
| 287 | ZD178- 63-8 (M8) | 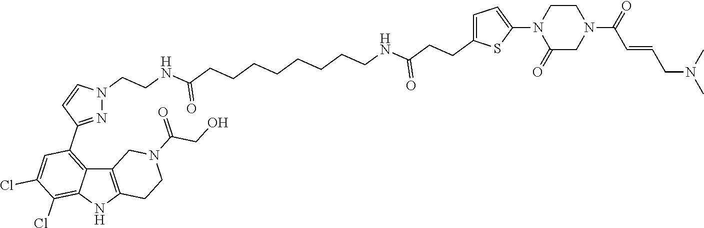 [SMILES: CN(C)C/C=C/C(=O)N1CCN(c2ccc(CCC(=O)NCCCCCCCCC(=O)NCCn3ccc(-c4cc(Cl)c(Cl)c5[nH]c6c(c45)CN(C(=O)CO)CC6)n3)s2)C(=O)C1] |
| 288 | ZD178- 63-9 (M9) | 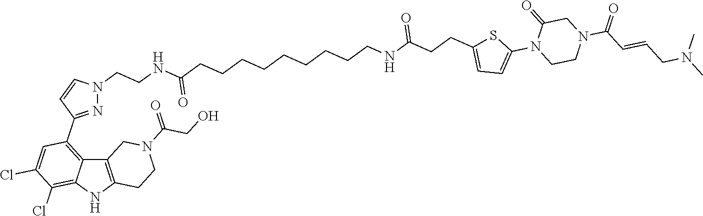 [SMILES: CN(C)C/C=C/C(=O)N1CCN(c2ccc(CCC(=O)NCCCCCCCCCC(=O)NCCn3ccc(-c4cc(Cl)c(Cl)c5[nH]c6c(c45)CN(C(=O)CO)CC6)n3)s2)C(=O)C1] |
| 289 | ZD178- 63-10 (M10) | 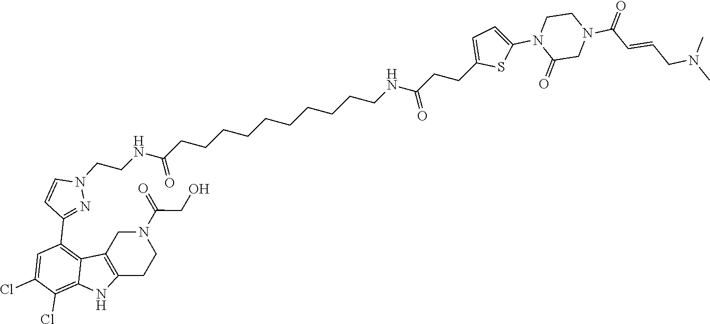 [SMILES: CN(C)C/C=C/C(=O)N1CCN(c2ccc(CCC(=O)NCCCCCCCCCCC(=O)NCCn3ccc(-c4cc(Cl)c(Cl)c5[nH]c6c(c45)CN(C(=O)CO)CC6)n3)s2)C(=O)C1] |
| 290 | ZD178- 63-11 (M11) | 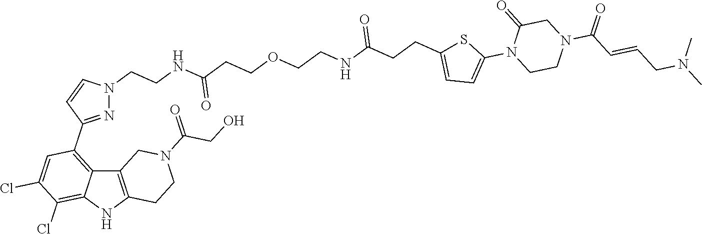 [SMILES: CN(C)C/C=C/C(=O)N1CCN(c2ccc(CCC(=O)NCCOCCC(=O)NCCn3ccc(-c4cc(Cl)c(Cl)c5[nH]c6c(c45)CN(C(=O)CO)CC6)n3)s2)C(=O)C1] |
| 291 | ZD178- 63-12 (M12) | 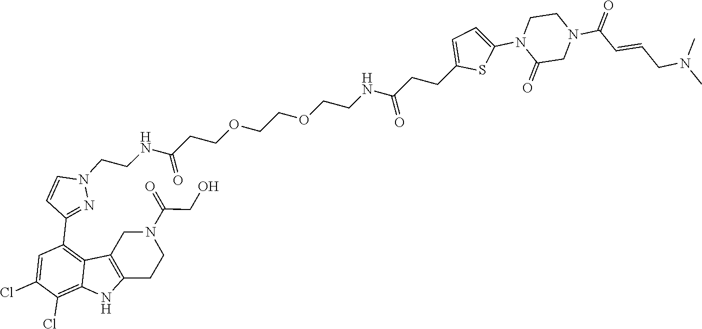 [SMILES: CN(C)C/C=C/C(=O)N1CCN(c2ccc(CCC(=O)NCCOCCOCCC(=O)NCCn3ccc(-c4cc(Cl)c(Cl)c5[nH]c6c(c45)CN(C(=O)CO)CC6)n3)s2)C(=O)C1] |
| 292 | ZD178- 63-13 (M13) | 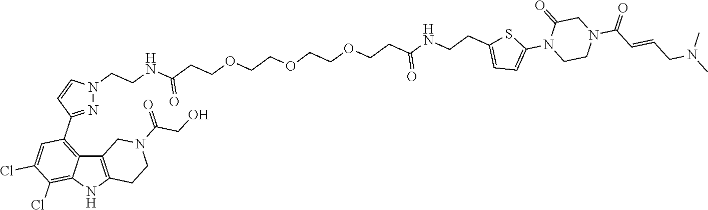 [SMILES: CN(C)C/C=C/C(=O)N1CCN(c2ccc(CCNC(=O)CCOCCOCCOCCC(=O)NCCn3ccc(-c4cc(Cl)c(Cl)c5[nH]c6c(c45)CN(C(=O)CO)CC6)n3)s2)C(=O)C1] |
| 293 | ZD178- 63-14 (M14) | 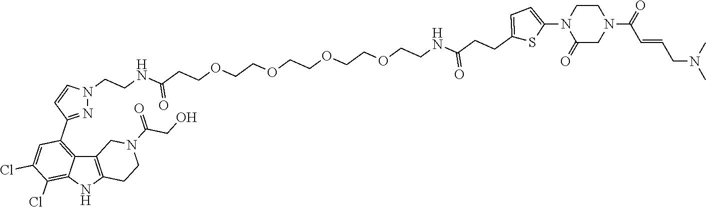 [SMILES: CN(C)C/C=C/C(=O)N1CCN(c2ccc(CCC(=O)NCCOCCOCCOCCOCCC(=O)NCCn3ccc(-c4cc(Cl)c(Cl)c5[nH]c6c(c45)CN(C(=O)CO)CC6)n3)s2)C(=O)C1] |
| 294 | ZD178- 63-15 (M15) | 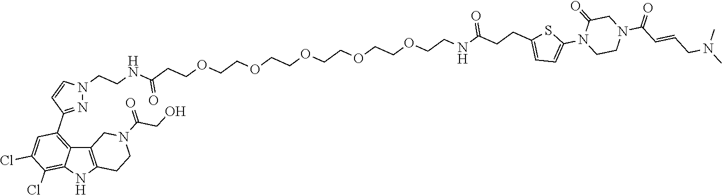 [SMILES: CN(C)C/C=C/C(=O)N1CCN(c2ccc(CCC(=O)NCCOCCOCCOCCOCCOCCC(=O)NCCn3ccc(-c4cc(Cl)c(Cl)c5[nH]c6c(c45)CN(C(=O)CO)CC6)n3)s2)C(=O)C1] |
| 295 | ZD178- 69-1 (X1) | 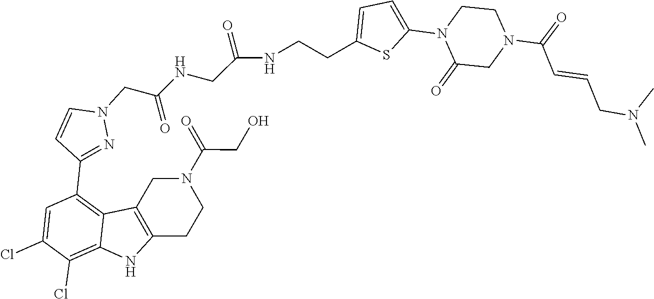 [SMILES: CN(C)C/C=C/C(=O)N1CCN(c2ccc(CCNC(=O)CNC(=O)Cn3ccc(-c4cc(Cl)c(Cl)c5[nH]c6c(c45)CN(C(=O)CO)CC6)n3)s2)C(=O)C1] |
| 296 | ZD178- 69-2 (X2) | 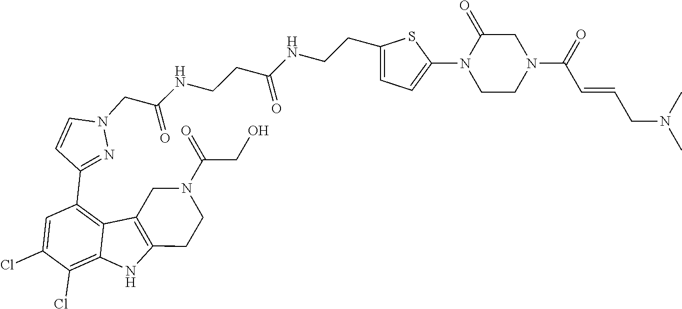 [SMILES: CN(C)C/C=C/C(=O)N1CCN(c2ccc(CCNC(=O)CCNC(=O)Cn3ccc(-c4cc(Cl)c(Cl)c5[nH]c6c(c45)CN(C(=O)CO)CC6)n3)s2)C(=O)C1] |
| 297 | ZD178- 69-3 (X3) | 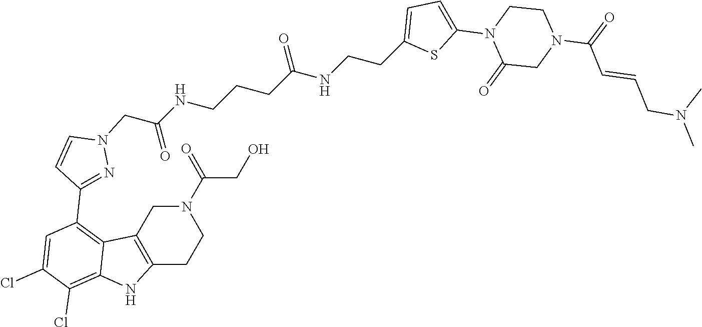 [SMILES: CN(C)C/C=C/C(=O)N1CCN(c2ccc(CCNC(=O)CCCNC(=O)Cn3ccc(-c4cc(Cl)c(Cl)c5[nH]c6c(c45)CN(C(=O)CO)CC6)n3)s2)C(=O)C1] |
| 298 | ZD178- 69-4 (X4) | 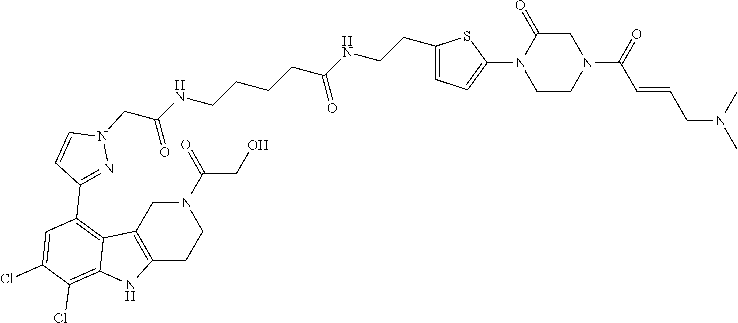 [SMILES: CN(C)C/C=C/C(=O)N1CCN(c2ccc(CCNC(=O)CCCCNC(=O)Cn3ccc(-c4cc(Cl)c(Cl)c5[nH]c6c(c45)CN(C(=O)CO)CC6)n3)s2)C(=O)C1] |
| 299 | ZD178- 69-5 (X5) | 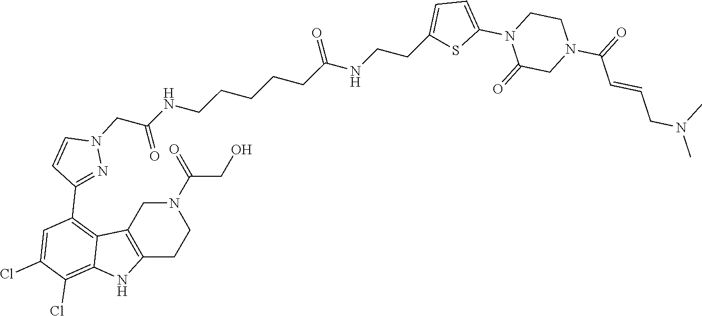 [SMILES: CN(C)C/C=C/C(=O)N1CCN(c2ccc(CCNC(=O)CCCCCNC(=O)Cn3ccc(-c4cc(Cl)c(Cl)c5[nH]c6c(c45)CN(C(=O)CO)CC6)n3)s2)C(=O)C1] |
| 300 | ZD178- 69-6 (X6) | 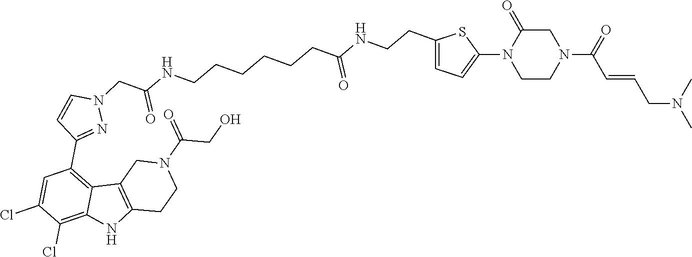 [SMILES: CN(C)C/C=C/C(=O)N1CCN(c2ccc(CCNC(=O)CCCCCCNC(=O)Cn3ccc(-c4cc(Cl)c(Cl)c5[nH]c6c(c45)CN(C(=O)CO)CC6)n3)s2)C(=O)C1] |
| 301 | ZD178- 69-7 (X7) | 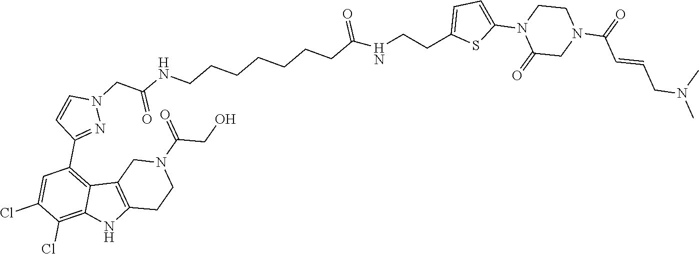 [SMILES: CN(C)C/C=C/C(=O)N1CCN(c2ccc(CCNC(=O)CCCCCCCNC(=O)Cn3ccc(-c4cc(Cl)c(Cl)c5[nH]c6c(c45)CN(C(=O)CO)CC6)n3)s2)C(=O)C1] |
| 302 | ZD178- 69-8 (X8) | 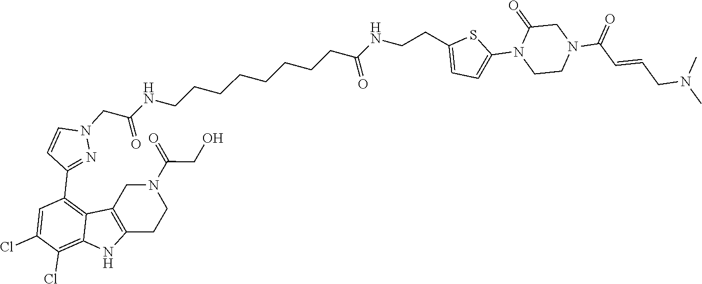 [SMILES: CN(C)C/C=C/C(=O)N1CCN(c2ccc(CCNC(=O)CCCCCCCCNC(=O)Cn3ccc(-c4cc(Cl)c(Cl)c5[nH]c6c(c45)CN(C(=O)CO)CC6)n3)s2)C(=O)C1] |
| 303 | ZD178- 69-9 (X9) | 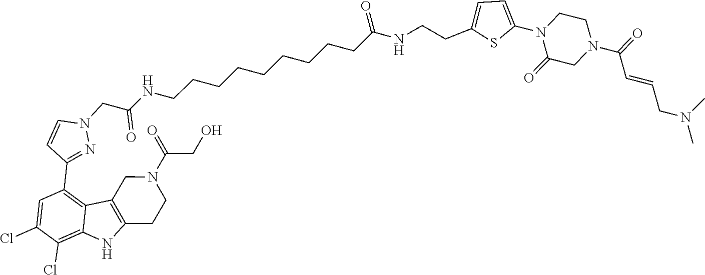 [SMILES: CN(C)C/C=C/C(=O)N1CCN(c2ccc(CCNC(=O)CCCCCCCCCNC(=O)Cn3ccc(-c4cc(Cl)c(Cl)c5[nH]c6c(c45)CN(C(=O)CO)CC6)n3)s2)C(=O)C1] |
| 304 | ZD178- 69-10 (X10) | 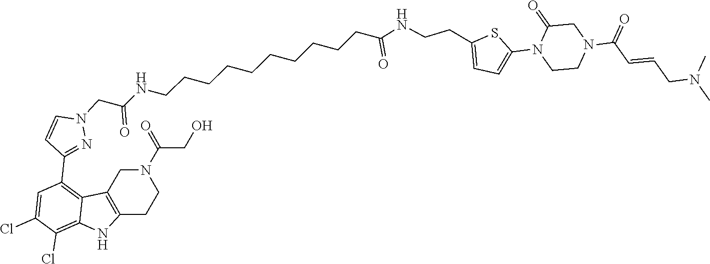 [SMILES: CN(C)C/C=C/C(=O)N1CCN(c2ccc(CCNC(=O)CCCCCCCCCCNC(=O)Cn3ccc(-c4cc(Cl)c(Cl)c5[nH]c6c(c45)CN(C(=O)CO)CC6)n3)s2)C(=O)C1] |
| 305 | ZD178- 69-11 (X11) | 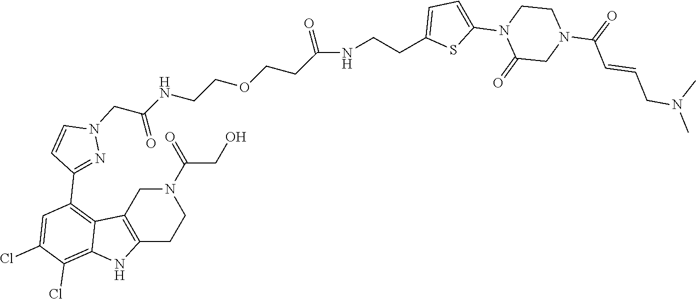 [SMILES: CN(C)C/C=C/C(=O)N1CCN(c2ccc(CCNC(=O)CCOCCNC(=O)Cn3ccc(-c4cc(Cl)c(Cl)c5[nH]c6c(c45)CN(C(=O)CO)CC6)n3)s2)C(=O)C1] |
| 306 | ZD178- 69-12 (X12) | 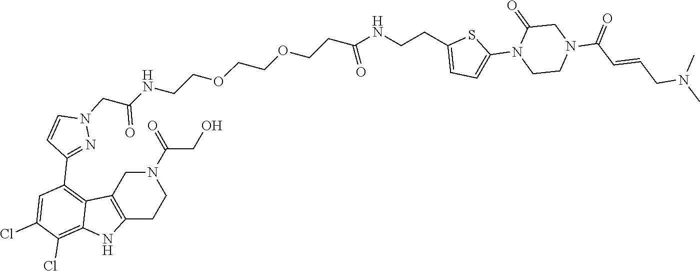 [SMILES: CN(C)C/C=C/C(=O)N1CCN(c2ccc(CCNC(=O)CCOCCOCCNC(=O)Cn3ccc(-c4cc(Cl)c(Cl)c5[nH]c6c(c45)CN(C(=O)CO)CC6)n3)s2)C(=O)C1] |
| 307 | ZD178- 69-13 (X13) | 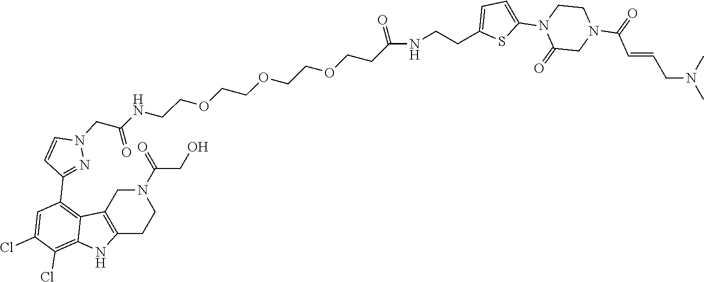 [SMILES: CN(C)C/C=C/C(=O)N1CCN(c2ccc(CCNC(=O)CCOCCOCCOCCNC(=O)Cn3ccc(-c4cc(Cl)c(Cl)c5[nH]c6c(c45)CN(C(=O)CO)CC6)n3)s2)C(=O)C1] |
| 308 | ZD178- 69-14 (X14) | 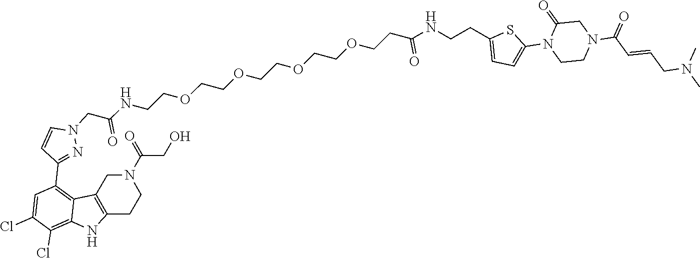 [SMILES: CN(C)C/C=C/C(=O)N1CCN(c2ccc(CCNC(=O)CCOCCOCCOCCOCCNC(=O)Cn3ccc(-c4cc(Cl)c(Cl)c5[nH]c6c(c45)CN(C(=O)CO)CC6)n3)s2)C(=O)C1] |
| 309 | ZD178- 69-15 (X15) | 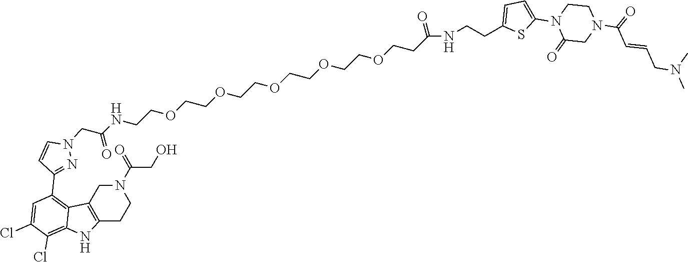 [SMILES: CN(C)C/C=C/C(=O)N1CCN(c2ccc(CCNC(=O)CCOCCOCCOCCOCCOCCNC(=O)Cn3ccc(-c4cc(Cl)c(Cl)c5[nH]c6c(c45)CN(C(=O)CO)CC6)n3)s2)C(=O)C1] |
| | Example | |
| | No. | name |
| | 252 | (E)-N-(2-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)ethyl)-3-(5-(4-(4-(dimethylamino)but-2-enoyl)-2-oxopiperazin-1-yl)thiophen-2-yl)propenamide |
| | 253 | (E)-N-(3-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)propyl)-3-(5-(4-(4-(dimethylamino)but-2-enoyl)-2-oxopiperazin-1-yl)thiophen-2-yl)propenamide |
| | 254 | (E)-N-(4-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)butyl)-3-(5-(4-(4-(dimethylamino)but-2-enoyl)-2-oxopiperazin-1-yl)thiophen-2-yl)propenamide |
| | 255 | (E)-N-(5-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)pentyl)-3-(5-(4-(4-(dimethylamino)but-2-enoyl)-2-oxopiperazin-1-yl)thiophen-2-yl)propanamide |
| | 256 | (E)-N-(6-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)hexyl)-3-(5-(4-(4-(dimethylamino)but-2-enoyl)-2-oxopiperazin-1-yl)thiophen-2-yl)propenamide |
| | 257 | (E)-N-(7-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)heptyl)-3-(5-(4-(4-(dimethylamino)but-2-enoyl)-2-oxopiperazin-1-yl)thiophen-2-yl)propenamide |
| | 258 | (E)-N-(8-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)octyl)-3-(5-(4-(4-(dimethylamino)but-2-enoyl)-2-oxopiperazin-1-yl)thiophen-2-yl)propenamide |
| | 259 | (E)-N-(9-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)nonyl)-3-(5-(4-(4-(dimethylamino)but-2-enoyl)-2-oxopiperazin-1-yl)thiophen-2-yl)propenamide |
| | 260 | (E)-N-(10-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)decyl)-3-(5-(4-(4-(dimethylamino)but-2-enoyl)-2-oxopiperazin-1-yl)thiophen-2-yl)propenamide |
| | 261 | (E)-N-(2-(2-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)ethoxy)ethyl)-3-(5-(4-(4-(dimethylamino)but-2-enoyl)-2-oxopiperazin-1-yl)thiophen-2-yl)propenamide |
| | 262 | (E)-N-(2-(2-(2-(2-(3-(6,7-dichloro-2-(2-hydroxyacetyl)-2,3,4,5-tetrahydro-1H-pyrido[4,3-b]indol-9-yl)-1H-pyrazol- |
| | | 1-yl)acetamido)ethoxy)ethoxy)ethyl)-3-(5-(4-(4-(dimethylamino)but-2-enoyl)-2-oxopiperazin-1-yl)thiophen-2- |
| | | yl)propenamide |
| | 263 | (E)-N-(1-(3-(6,7-dichloro-2-(2-hydroxyacetyl)-2,3,4,5-tetrahydro-1H-pyrido[4,3-b]indol-9-yl)-1H-pyrazol-1-yl)-2- |
| | | oxo-6,9,12-trioxa-3-azatetradecan-14-yl)-3-(5-(4-(4-(dimethylamino)but-2-enoyl)-2-oxopiperazin-1-yl)thiophen- |
| | | 2-yl)propenamide |
| | 264 | (E)-N-(1-(3-(6,7-dichloro-2-(2-hydroxyacetyl)-2,3,4,5-tetrahydro-1H-pyrido[4,3-b]indol-9-yl)-1H-pyrazol-1-yl)-2- |
| | | oxo-6,9,12,15-tetraoxa-3-azaheptadecan-17-yl)-3-(5-(4-(4-(dimethylamino)but-2-enoyl)-2-oxopiperazin-1-yl)thiophen- |
| | | 2-yl)propenamide |
| | 265 | (E)-N-(1-(3-(6,7-dichloro-2-(2-hydroxyacetyl)-2,3,4,5-tetrahydro-1H-pyrido[4,3-b]indol-9-yl)-1H-pyrazol-1-yl)-2-oxo- |
| | | 6,9,12,15,18-pentaoxa-3-azaicosan-20-yl)-3-(5-(4-(4-(dimethylamino)but-2-enoyl)-2-oxopiperazin-1-yl)thiophen- |
| | | 2-yl)propenamide |
| | 266 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N4-(2-(5-(4-(4-(dimethylamino)but-2-enoyl)-2-oxopiperazin-1-yl)thiophen-2-yl)ethyl)succinamide |
| | 267 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N5-(2-(5-(4-(4-(dimethylamino)but-2-enoyl)-2-oxopiperazin-1-yl)thiophen-2-yl)ethyl)glutaramide |
| | 268 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N6-(2-(5-(4-(4-(dimethylamino)but-2-enoyl)-2-oxopiperazin-1-yl)thiophen-2-yl)ethyl)adipamide |
| | 269 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N7-(2-(5-(4-(4-(dimethylamino)but-2-enoyl)-2-oxopiperazin-1-yl)thiophen-2-yl)ethyl)heptanediamide |
| | 270 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N8-(2-(5-(4-(4-(dimethylamino)but-2-enoyl)-2-oxopiperazin-1-yl)thiophen-2-yl)ethyl)octanediamide |
| | 271 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N9-(2-(5-(4-(4-(dimethylamino)but-2-enoyl)-2-oxopiperazin-1-yl)thiophen-2-yl)ethyl)nonanediamide |
| | 272 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N10-(2-(5-(4-(4-(dimethylamino)but-2-enoyl)-2-oxopiperazin-1-yl)thiophen-2-yl)ethyl)decanediamide |
| | 273 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N11-(2-(5-(4-(4-(dimethylamino)but-2-enoyl)-2-oxopiperazin-1-yl)thiophen-2-yl)ethyl)undecanediamide |
| | 274 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N12-(2-(5-(4-(4-(dimethylamino)but-2-enoyl)-2-oxopiperazin-1-yl)thiophen-2-yl)ethyl)dodecanediamide |
| | 275 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 3-(3-((2-(5-(4-(4-(dimethylamino)but-2-enoyl)-2-oxopiperazin-1-yl)thiophen-2-yl)ethyl)amino)-3-oxopropoxy)propenamide |
| | 276 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 3-(2-(3-((2-(5-(4-(4-(dimethylamino)but-2-enoyl)-2-oxopiperazin-1-yl)thiophen-2-yl)ethyl)amino)-3- |
| | | oxopropoxy)ethoxy)propenamide |
| | 277 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 3-(2-(2-(3-((2-(5-(4-(4-(dimethylamino)but-2-enoyl)-2-oxopiperazin-1-yl)thiophen-2-yl)ethyl)amino)-3-oxopropoxy) |
| | | ethoxy)ethoxy)propenamide |
| | 278 | (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | N16-(2-(5-(4-(4-(dimethylamino)but-2-enoyl)-2-oxopiperazin-1-yl)thiophen-2-yl)ethyl)-4,7,10,13-tetraoxahexadecanediamide |
| | 279 | (E)-N1-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)ethyl)-N19-(2-(5-(4-(4-(dimethylamino)but-2-enoyl)-2-oxopiperazin-1-yl)thiophen-2-yl)ethyl)-4,7,10,13,16- |
| | | pentaoxanonadecanediamide |
| | 280 | (E)-N-(2-((2-(3-(6,7-dichloro-2-(2-hydroxyacetyl)-2,3,4,5-tetrahydro-1H-pyrido[4,3-b]indol-9-yl)-1H-pyrazol-1- |
| | | yl)ethyl)amino)-2-oxoethyl)-3-(5-(4-(4-(dimethylamino)but-2-enoyl)-2-oxopiperazin-1-yl)thiophen-2-yl)propenamide |
| | 281 | (E)-N-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)ethyl)-3-(3-(5-(4-(4-(dimethylamino)but-2-enoyl)-2-oxopiperazin-1-yl)thiophen-2-yl)propanamido)propenamide |
| | 282 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | ethyl)-4-(3-(5-(4-(4-(dimethylamino)but-2-enoyl)-2-oxopiperazin-1-yl)thiophen-2-yl)propanamido)butanamide |
| | 283 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 5-(3-(5-(4-(4-(dimethylamino)but-2-enoyl)-2-oxopiperazin-1-yl)thiophen-2-yl)propanamido)pentanamide |
| | 284 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | ethyl)-6-(3-(5-(4-(4-(dimethylamino)but-2-enoyl)-2-oxopiperazin-1-yl)thiophen-2-yl)propanamido)hexanamide |
| | 285 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 7-(3-(5-(4-(4-(dimethylamino)but-2-enoyl)-2-oxopiperazin-1-yl)thiophen-2-yl)propanamido)heptanamide |
| | 286 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 8-(3-(5-(4-(4-(dimethylamino)but-2-enoyl)-2-oxopiperazin-1-yl)thiophen-2-yl)propanamido)octanamide |
| | 287 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 9-(3-(5-(4-(4-(dimethylamino)but-2-enoyl)-2-oxopiperazin-1-yl)thiophen-2-yl)propanamido)nonanamide |
| | 288 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 10-(3-(5-(4-(4-(dimethylamino)but-2-enoyl)-2-oxopiperazin-1-yl)thiophen-2-yl)propanamido)decanamide |
| | 289 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 11-(3-(5-(4-(4-(dimethylamino)but-2-enoyl)-2-oxopiperazin-1-yl)thiophen-2-yl)propanamido)undecanamide |
| | 290 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 3-(2-(3-(5-(4-(4-(dimethylamino)but-2-enoyl)-2-oxopiperazin-1-yl)thiophen-2-yl)propanamido)ethoxy)propenamide |
| | 291 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 3-(2-(2-(3-(5-(4-(4-(dimethylamino)but-2-enoyl)-2-oxopiperazin-1-yl)thiophen-2-yl)propanamido)ethoxy)ethoxy) |
| | | propenamide |
| | 292 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 3-(2-(2-(3-((2-(5-(4-(4-(dimethylamino)but-2-enoyl)-2-oxopiperazin-1-yl)thiophen-2-yl)ethyl)amino)-3-oxopropoxy) |
| | | ethoxy)ethoxy)propenamide |
| | 293 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 1-(3-(5-(4-(4-(dimethylamino)but-2-enoyl)-2-oxopiperazin-1-yl)thiophen-2-yl)propanamido)-3,6,9,12-tetraoxapentadecan- |
| | | 15-amide |
| | 294 | (E)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)- |
| | | 1-(3-(5-(4-(4-(dimethylamino)but-2-enoyl)-2-oxopiperazin-1-yl)thiophen-2-yl)propanamido)-3,6,9,12,15- |
| | | pentaoxaoctadecan-18-amide |
| | 295 | (E)-2-(3-(6,7-dichloro-2-(2-hydroxyacetyl)-2,3,4,5-tetrahydro-1H-pyrido[4,3-b]indol-9-yl)-1H-pyrazol-1-yl)-N-(2- |
| | | ((2-(5-(4-(4-(dimethylamino)but-2-enoyl)-2-oxopiperazin-1-yl)thiophen-2-yl)ethyl)amino)-2-oxoethyl)acetamide |
| | 296 | (E)-3-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)-N-(2-(5-(4-(4-(dimethylamino)but-2-enoyl)-2-oxopiperazin-1-yl)thiophen-2-yl)ethyl)propenamide |
| | 297 | (E)-4-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)-N-(2-(5-(4-(4-(dimethylamino)but-2-enoyl)-2-oxopiperazin-1-yl)thiophen-2-yl)ethyl)butanamide |
| | 298 | (E)-5-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)-N-(2-(5-(4-(4-(dimethylamino)but-2-enoyl)-2-oxopiperazin-1-yl)thiophen-2-yl)ethyl)pentanamide |
| | 299 | (E)-6-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)-N-(2-(5-(4-(4-(dimethylamino)but-2-enoyl)-2-oxopiperazin-1-yl)thiophen-2-yl)ethyl)hexanamide |
| | 300 | (E)-7-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)-N-(2-(5-(4-(4-(dimethylamino)but-2-enoyl)-2-oxopiperazin-1-yl)thiophen-2-yl)ethyl)heptanamide |
| | 301 | (E)-8-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)-N-(2-(5-(4-(4-(dimethylamino)but-2-enoyl)-2-oxopiperazin-1-yl)thiophen-2-yl)ethyl)octanamide |
| | 302 | (E)-9-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)-N-(2-(5-(4-(4-(dimethylamino)but-2-enoyl)-2-oxopiperazin-1-yl)thiophen-2-yl)ethyl)nonanamide |
| | 303 | (E)-10-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)-N-(2-(5-(4-(4-(dimethylamino)but-2-enoyl)-2-oxopiperazin-1-yl)thiophen-2-yl)ethyl)decanamide |
| | 304 | (E)-11-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)-N-(2-(5-(4-(4-(dimethylamino)but-2-enoyl)-2-oxopiperazin-1-yl)thiophen-2-yl)ethyl)undecanamide |
| | 305 | (E)-3-(2-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)ethoxy)-N-(2-(5-(4-(4-(dimethylamino)but-2-enoyl)-2-oxopiperazin-1-yl)thiophen-2-yl)ethyl)propenamide |
| | 306 | (E)-3-(2-(2-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)ethoxy)ethoxy)-N-(2-(5-(4-(4-(dimethylamino)but-2-enoyl)-2-oxopiperazin-1-yl)thiophen-2-yl)ethyl) |
| | | propenamide |
| | 307 | (E)-3-(2-(2-(2-(2-(3-(6,7-dichloro-2-(2-hydroxyacetyl)-2,3,4,5-tetrahydro-1H-pyrido[4,3-b]indol-9-yl)-1H-pyrazol-1- |
| | | yl)acetamido)ethoxy)ethoxy)ethoxy)-N-(2-(5-(4-(4-(dimethylamino)but-2-enoyl)-2-oxopiperazin-1-yl)thiophen-2-yl) |
| | | ethyl)propenamide |
| | 308 | (E)-1-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)-N-(2-(5-(4-(4-(dimethylamino)but-2-enoyl)-2-oxopiperazin-1-yl)thiophen-2-yl)ethyl)-3,6,9,12- |
| | | tetraoxapentadecan-15-amide |
| | 309 | (E)-1-(2-(3-(6,7-dichloro-2-(2-hydroxyacetyl)-2,3,4,5-tetrahydro-1H-pyrido[4,3-b]indol-9-yl)-1H-pyrazol-1-yl) |
| | | acetamido)-N-(2-(5-(4-(4-(dimethylamino)but-2-enoyl)-2-oxopiperazin-1-yl)thiophen-2-yl)ethyl)-3,6,9,12,15- |
| | | pentaoxaoctadecan-18-amide |

Examples of CFTR based bivalent compounds are set forth in table 4 below.

TABLE 4

| | Note | | |
|---|---|---|---|
| Examples | ID | Structure | Chemical Name |
| 310 | QC166- 130 (Q1) | 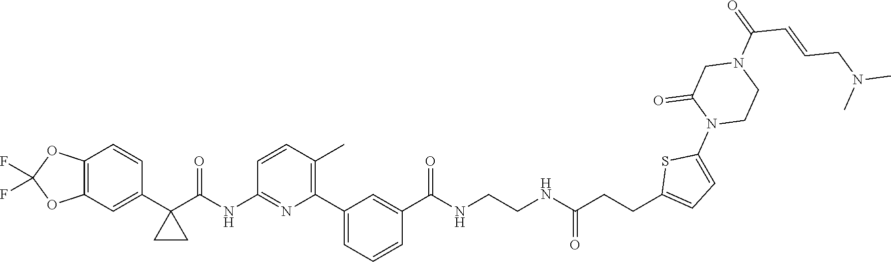 [SMILES: Cc1ccc(NC(=O)C2(c3ccc4c(c3)OC(F)(F)O4)CC2)nc1-c1cccc(C(=O)NCCNC(=O)CCc2ccc(N3CCN(C(=O)/C=C/CN(C)C)CC3=O)s2)c1] | (E)-3-(6-(1-(2,2- difluorobenzo[d][1,3] dioxol-5- yl)cyclopropane-1- carboxamido)-3- methylpyridin-2-yl)- N-(2-(3-(5-(4-(4- (dimethylamino)but- 2-enoyl)-2- oxopiperazin-1- yl)thiophen-2- yl)propanamido)ethyl) benzamide |
| 311 | QC166- 131 (Q2) | 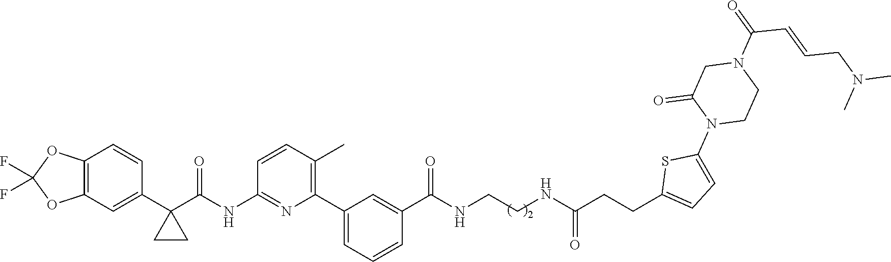 | (E)-3-(6-(1-(2,2- difluorobenzo[d][1,3] dioxol-5- yl)cyclopropane-1- carboxamido)-3- methylpyridin-2-yl)- N-(3-(3-(5-(4-(4- (dimethylamino)but- 2-enoyl)-2- oxopiperazin-1- yl)thiophen-2- yl)propanamido) propyl)benzamide |
| 312 | QC166- 132 (Q3) | 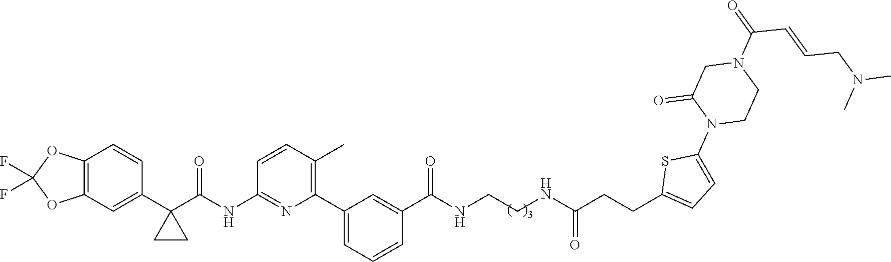 | (E)-3-(6-(1-(2,2- difluorobenzo[d][1,3] dioxol-5- yl)cyclopropane-1- carboxamido)-3- methylpyridin-2-yl)- N-(4-(3-(5-(4-(4- (dimethylamino)but- 2-enoyl)-2- oxopiperazin-1- yl)thiophen-2- yl)propanamido)butyl) benzamide |
| 313 | QC166- 133 (Q4) | 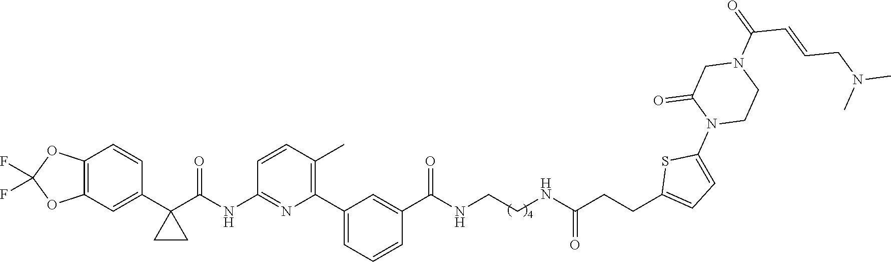 | E)-3-(6-(1-(2,2- difluorobenzo[d][1,3] dioxol-5- yl)cyclopropane-1- carboxamido)-3- methylpyridin-2-yl)- N-(5-(3-(5-(4-(4- (dimethylamino)but- 2-enoyl)-2- oxopiperazin-1- yl)thiophen-2- yl)propanamido)pentyl) benzamide |
| 314 | QC166- 134 (Q5) | 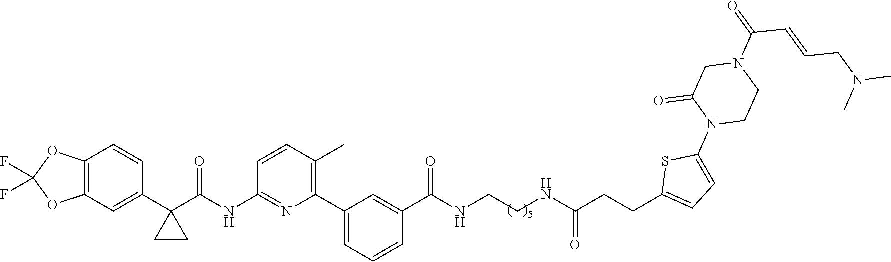 | (E)-3-(6-(1-(2,2- difluorobenzo[d][1,3] dioxol-5- yl)cyclopropane-1- carboxamido)-3- methylpyridin-2-yl)- N-(6-(3-(5-(4-(4- (dimethylamino)but- 2-enoyl)-2- oxopiperazin-1- yl)thiophen-2- yl)propanamido)hexyl) benzamide |
| 315 | QC166- 135 (Q6) | 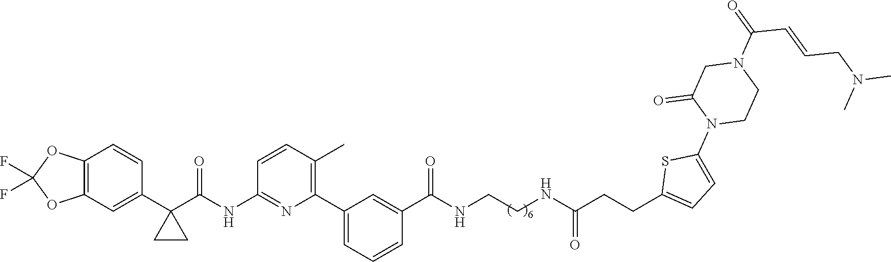 | (E)-3-(6-(1-(2,2- difluorobenzo[d][1,3] dioxol-5- yl)cyclopropane-1- carboxamido)-3- methylpyridin-2-yl)- N-(7-(3-(5-(4-(4- (dimethylamino)but- 2-enoyl)-2- oxopiperazin-1- yl)thiophen-2- yl)propanamido)heptyl) benzamide |
| 316 | QC166- 136 (Q7) | 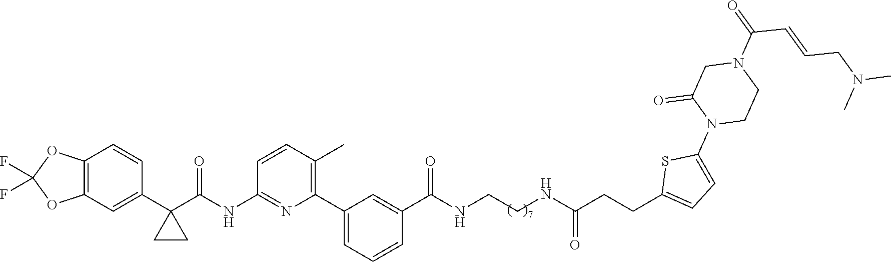 | (E)-3-(6-(1-(2,2- difluorobenzo[d][1,3] dioxol-5- yl)cyclopropane-1- carboxamido)-3- methylpyridin-2-yl)- N-(8-(3-(5-(4-(4- (dimethylamino)but- 2-enoyl)-2- oxopiperazin-1- yl)thiophen-2- yl)propanamido)octyl) benzamide |
| 317 | QC166- 137 | 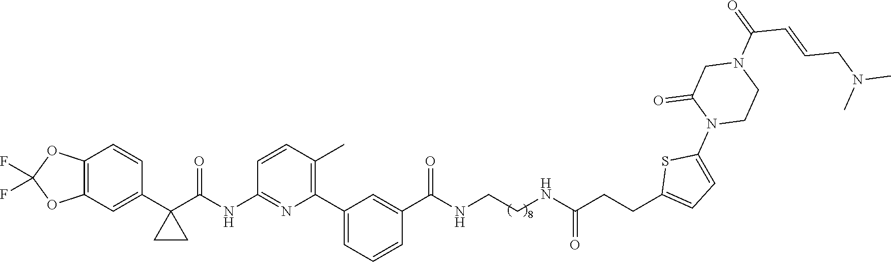 | (E)-3-(6-(1-(2,2- difluorobenzo[d][1,3] dioxol-5- yl)cyclopropane-1- carboxamido)-3- methylpyridin-2-yl)- N-(9-(3-(5-(4-(4- (dimethylamino)but- 2-enoyl)-2- oxopiperazin-1- yl)thiophen-2- yl)propanamido)nonyl) benzamide |
| 318 | QC166- 138 | 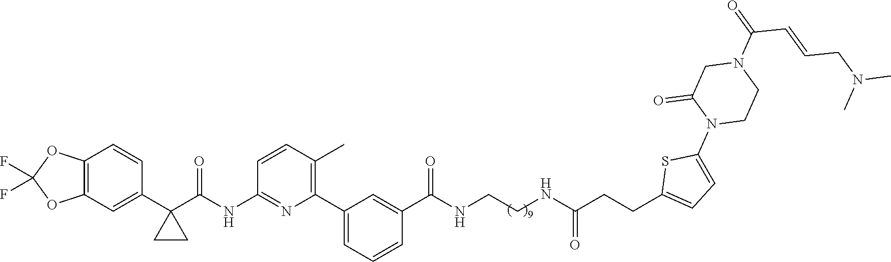 | (E)-3-(6-(1-(2,2- difluorobenzo[d][1,3] dioxol-5- yl)cyclopropane-1- carboxamido)-3- methylpyridin-2-yl)- N-(10-(3-(5-(4-(4- (dimethylamino)but- 2-enoyl)-2- oxopiperazin-1- yl)thiophen-2- yl)propanamido)decyl) benzamide |
| 319 | QC166- 139 (Q8) | 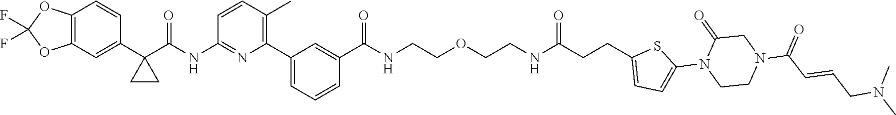 [SMILES: Cc1ccc(NC(=O)C2(c3ccc4c(c3)OC(F)(F)O4)CC2)nc1-c1cccc(C(=O)NCCOCCNC(=O)CCc2ccc(N3CCN(C(=O)/C=C/CN(C)C)CC3=O)s2)c1] | (E)-3-(6-(1-(2,2- difluorobenzo[d][1,3] dioxol-5- yl)cyclopropane-1- carboxamido)-3- methylpyridin-2-yl)- N-(2-(2-(3-(5-(4-(4- (dimethylamino)but- 2-enoyl)-2- oxopiperazin-1- yl)thiophen-2- yl)propanamido)ethoxy) ethyl)benzamide |
| 320 | QC166- 140 (Q9) | 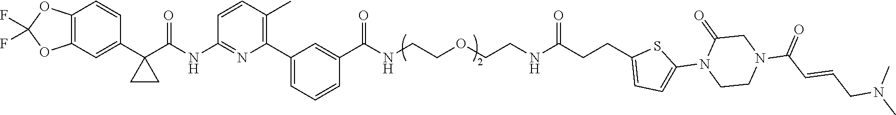 | (E)-3-(6-(1-(2,2- difluorobenzo[d][1,3] dioxol-5- yl)cyclopropane-1- carboxamido)-3- methylpyridin-2-yl)- N-(2-(2-(2-(3-(5-(4-(4- (dimethylamino)but- 2-enoyl)-2- oxopiperazin-1- yl)thiophen-2- yl)propanamido)ethoxy) ethoxy)ethyl) benzamide |
| 321 | QC166- 141 (Q10) | 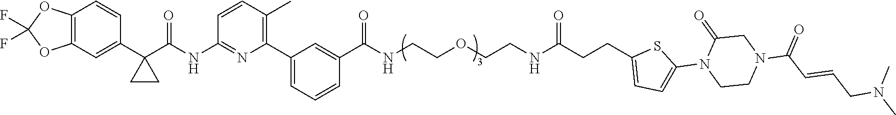 | (E)-3-(6-(1-(2,2- difluorobenzo[d][1,3] dioxol-5- yl)cyclopropane-1- carboxamido)-3- methylpyridin-2-yl)- N-(15-(5-(4-(4- (dimethylamino)but- 2-enoyl)-2- oxopiperazin-1- yl)thiophen-2-yl)-13- oxo-3,6,9-trioxa-12- azapentadecyl) benzamide |
| 322 | QC166- 142 (Q11) | 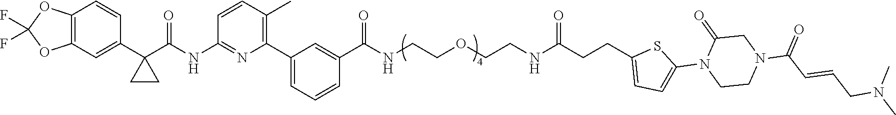 | (E)-3-(6-(1-(2,2- difluorobenzo[d][1,3] dioxol-5- yl)cyclopropane-1- carboxamido)-3- methylpyridin-2-yl)- N-(18-(5-(4-(4- (dimethylamino)but- 2-enoyl)-2- oxopiperazin-1- yl)thiophen-2-yl)-16- oxo-3,6,9,12-tetraoxa- 15- azaoctadecyl) benzamide |
| 323 | QC166- 143 (Q12) | 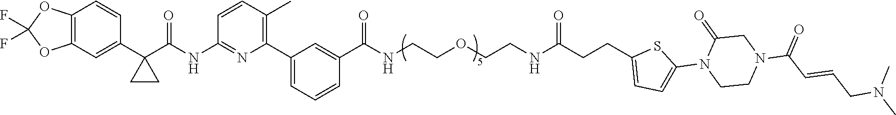 | (E)-3-(6-(1-(2,2- difluorobenzo[d][1,3] dioxol-5- yl)cyclopropane-1- carboxamido)-3- methylpyridin-2-yl)- N-(21-(5-(4-(4- (dimethylamino)but- 2-enoyl)-2- oxopiperazin-1- yl)thiophen-2-yl)-19- oxo-3,6,9,12,15- pentaoxa-18- azahenicosyl) benzamide |
| 324 | QC166- 167 (G1) | 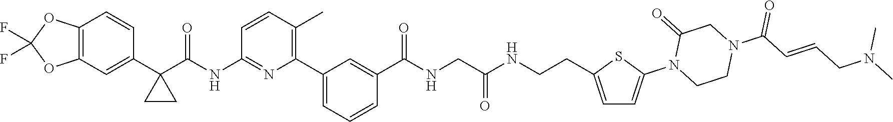 [SMILES: Cc1ccc(NC(=O)C2(c3ccc4c(c3)OC(F)(F)O4)CC2)nc1-c1cccc(C(=O)NCC(=O)NCCc2ccc(N3CCN(C(=O)/C=C/CN(C)C)CC3=O)s2)c1] | (E)-3-(6-(1-(2,2- difluorobenzo[d][1,3] dioxol-5- yl)cyclopropane-1- carboxamido)-3- methylpyridin-2-yl)- N-(2-((2-(5-(4-(4- (dimethylamino)but- 2-enoyl)-2- oxopiperazin-1- yl)thiophen-2- yl)ethyl)amino)-2- oxoethyl)benzamide |
| 325 | QC166- 174 (G2) | 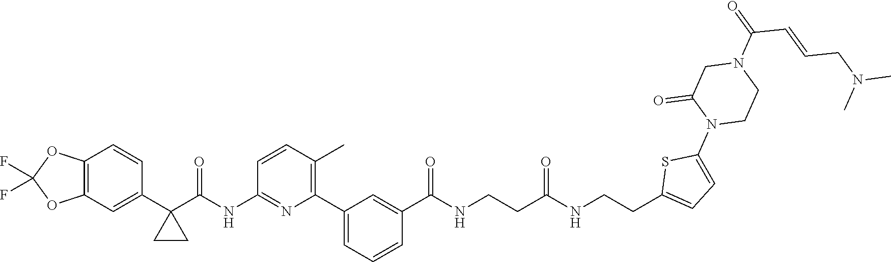 [SMILES: Cc1ccc(NC(=O)C2(c3ccc4c(c3)OC(F)(F)O4)CC2)nc1-c1cccc(C(=O)NCCC(=O)NCCc2ccc(N3CCN(C(=O)/C=C/CN(C)C)CC3=O)s2)c1] | (E)-3-(6-(1-(2,2- difluorobenzo[d][1,3] dioxol-5- yl)cyclopropane-1- carboxamido)-3- methylpyridin-2-yl)- N-(3-((2-(5-(4-(4- (dimethylamino)but- 2-enoyl)-2- oxopiperazin-1- yl)thiophen-2- yl)ethyl)amino)-3- oxopropyl)benzamide |
| 326 | QC166- 175 (G3) | 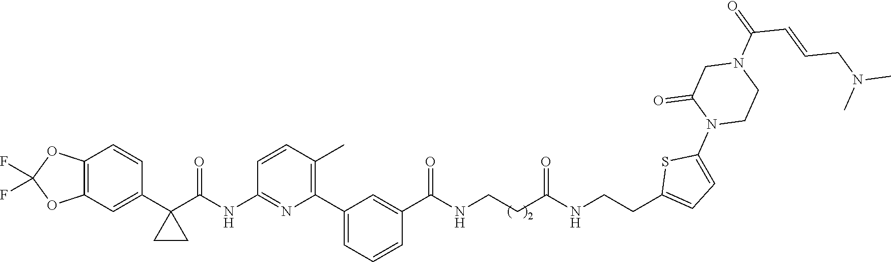 | (E)-3-(6-(1-(2,2- difluorobenzo[d][1,3] dioxol-5- yl)cyclopropane-1- carboxamido)-3- methylpyridin-2-yl)- N-(4-((2-(5-(4-(4- (dimethylamino)but- 2-enoyl)-2- oxopiperazin-1- yl)thiophen-2- yl)ethyl)amino)-4- oxobutyl)benzamide |
| 327 | QC166- 176 (G4) | 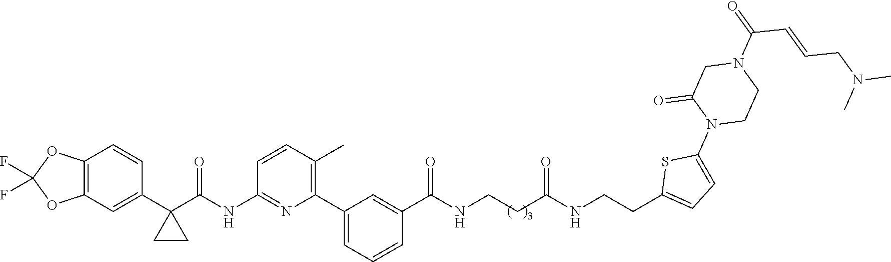 | (E)-3-(6-(1-(2,2- difluorobenzo[d][1,3] dioxol-5- yl)cyclopropane-1- carboxamido)-3- methylpyridin-2-yl)- N-(5-((2-(5-(4-(4- (dimethylamino)but- 2-enoyl)-2- oxopiperazin-1- yl)thiophen-2- yl)ethyl)amino)-5- oxopentyl)benzamide |
| 328 | QC166- 177 (G5) | 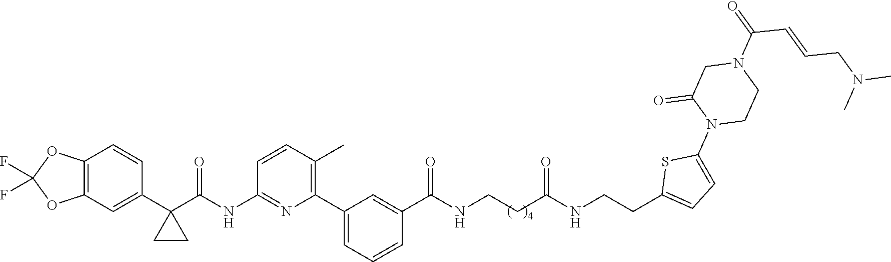 | (E)-3-(6-(1-(2,2- difluorobenzo[d][1,3] dioxol-5- yl)cyclopropane-1- carboxamido)-3- methylpyridin-2-yl)- N-(6-((2-(5-(4-(4- (dimethylamino)but- 2-enoyl)-2- oxopiperazin-1- yl)thiophen-2- yl)ethyl)amino)-6- oxohexyl)benzamide |
| 329 | QC166- 178 (MS6178) (G6) | 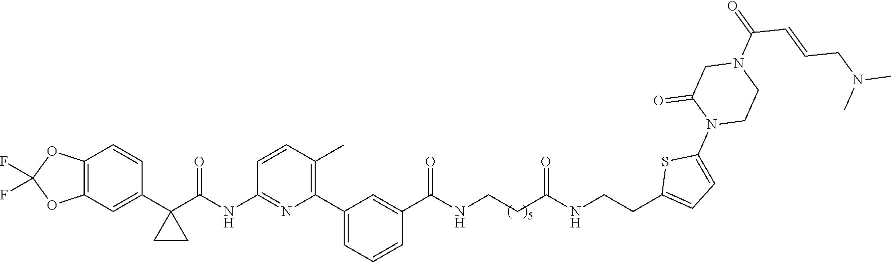 | (E)-3-(6-(1-(2,2- difluorobenzo[d][1,3] dioxol-5- yl)cyclopropane-1- carboxamido)-3- methylpyridin-2-yl)- N-(7-((2-(5-(4-(4- (dimethylamino)but- 2-enoyl)-2- oxopiperazin-1- yl)thiophen-2- yl)ethyl)amino)-7- oxoheptyl)benzamide |
| 330 | QC166- 179 (G7) | 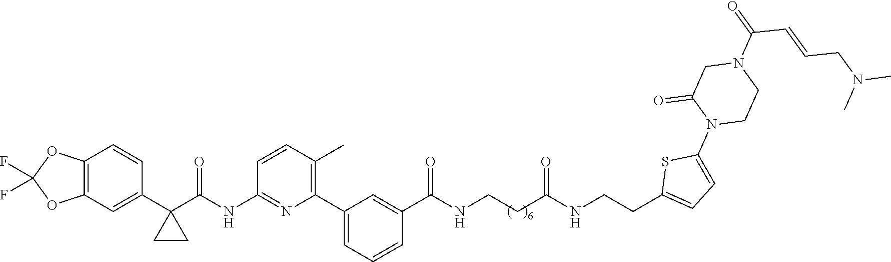 | (E)-3-(6-(1-(2,2- difluorobenzo[d][1,3] dioxol-5- yl)cyclopropane-1- carboxamido)-3- methylpyridin-2-yl)- N-(8-((2-(5-(4-(4- (dimethylamino)but- 2-enoyl)-2- oxopiperazin-1- yl)thiophen-2- yl)ethyl)amino)-8- oxooctyl)benzamide |
| 331 | QC166- 181 (G8) | 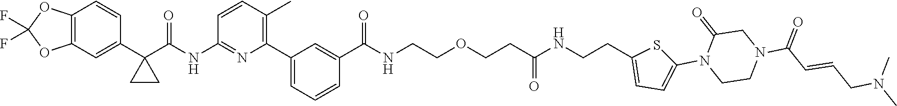 [SMILES: Cc1ccc(NC(=O)C2(c3ccc4c(c3)OC(F)(F)O4)CC2)nc1-c1cccc(C(=O)NCCOCCC(=O)NCCc2ccc(N3CCN(C(=O)/C=C/CN(C)C)CC3=O)s2)c1] | (E)-3-(6-(1-(2,2- difluorobenzo[d][1,3] dioxol-5- yl)cyclopropane-1- carboxamido)-3- methylpyridin-2-yl)- N-(2-(3-((2-(5-(4-(4- (dimethylamino)but- 2-enoyl)-2- oxopiperazin-1- yl)thiophen-2- yl)ethyl)amino)-3- oxopropoxy)ethyl) benzamide |
| 332 | QC166- 182 (G9) | 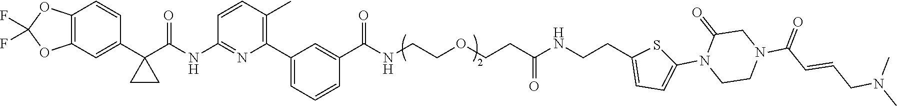 | (E)-3-(6-(1-(2,2- difluorobenzo[d][1,3] dioxol-5- yl)cyclopropane-1- carboxamido)-3- methylpyridin-2-yl)- N-(2-(2-(3-((2-(5-(4-(4- (dimethylamino)but- 2-enoyl)-2- oxopiperazin-1- yl)thiophen-2- yl)ethyl)amino)-3- oxopropoxy)ethoxy) ethyl)benzamide |
| 333 | QC166- 183 (G10) | 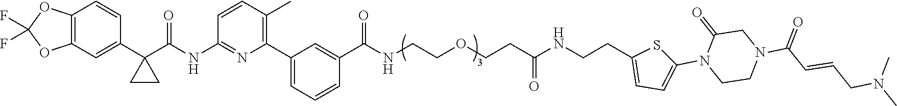 | (E)-3-(6-(1-(2,2- difluorobenzo[d][1,3] dioxol-5- yl)cyclopropane-1- carboxamido)-3- methylpyridin-2-yl)- N-(15-(5-(4-(4- (dimethylamino)but- 2-enoyl)-2- oxopiperazin-1- yl)thiophen-2-yl)-12- oxo-3,6,9-trioxa-13- azapentadecyl) benzamide |
| 334 | QC166- 184 (G11) | 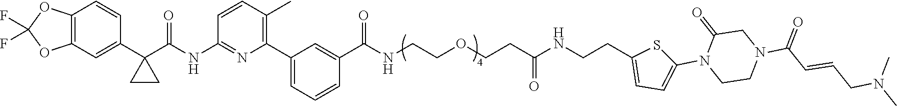 | (E)-3-(6-(1-(2,2- difluorobenzo[d][1,3] dioxol-5- yl)cyclopropane-1- carboxamido)-3- methylpyridin-2-yl)- N-(18-(5-(4-(4- (dimethylamino)but- 2-enoyl)-2- oxopiperazin-1- yl)thiophen-2-yl)-15- oxo-3,6,9,12-tetraoxa- 16- azaoctadecyl) benzamide |
| 335 | QC166- 185 (G12) | 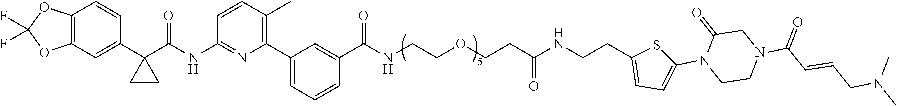 | (E)-3-(6-(1-(2,2- difluorobenzo[d][1,3] dioxol-5- yl)cyclopropane-1- carboxamido)-3- methylpyridin-2-yl)- N-(21-(5-(4-(4- (dimethylamino)but- 2-enoyl)-2- oxopiperazin-1- yl)thiophen-2-yl)-18- oxo-3,6,9,12,15- pentaoxa-19- azahenicosyl) benzamide |
| 336 | QC179- 104 | 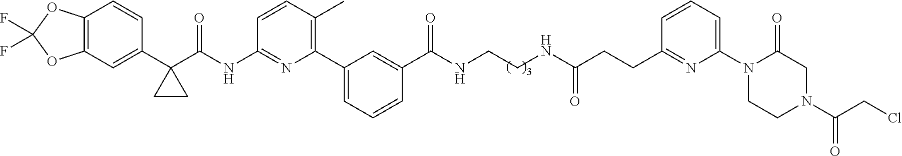 | N-(4-(3-(6-(4-(2- chloroacetyl)-2- oxopiperazin-1- yl)pyridin-2- yl)propanamido)butyl)- 3-(6-(1-(2,2- difluorobenzo[d][1,3] dioxol-5- yl)cyclopropane-1- carboxamido)-3- methylpyridin-2- yl)benzamide |
| 337 | QC179- 105 | 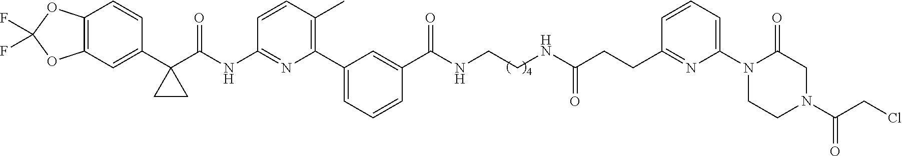 | N-(5-(3-(6-(4-(2- chloroacetyl)-2- oxopiperazin-1- yl)pyridin-2- yl)propanamido)pentyl)- 3-(6-(1-(2,2- difluorobenzo[d][1,3] dioxol-5- yl)cyclopropane-1- carboxamido)-3- methylpyridin-2- yl)benzamide |
| 338 | QC179- 106 | 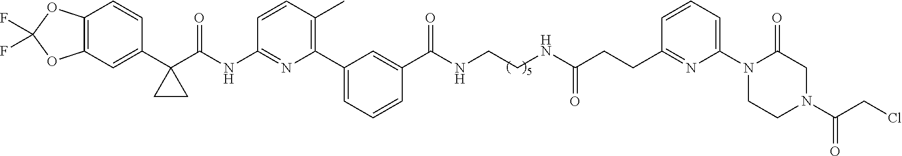 | N-(6-(3-(6-(4-(2- chloroacetyl)-2- oxopiperazin-1- yl)pyridin-2- yl)propanamido)hexyl)- 3-(6-(1-(2,2- difluorobenzo[d][1,3] dioxol-5- yl)cyclopropane-1- carboxamido)-3- methylpyridin-2- yl)benzamide |
| 339 | QC179- 107 | 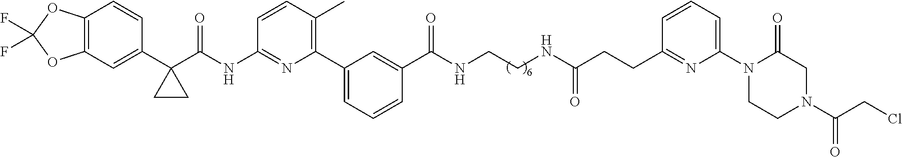 | N-(7-(3-(6-(4-(2- chloroacetyl)-2- oxopiperazin-1- yl)pyridin-2- yl)propanamido)heptyl)- 3-(6-(1-(2,2- difluorobenzo[d][1,3] dioxol-5- yl)cyclopropane-1- carboxamido)-3- methylpyridin-2- yl)benzamide |
| 340 | QC179- 108 | 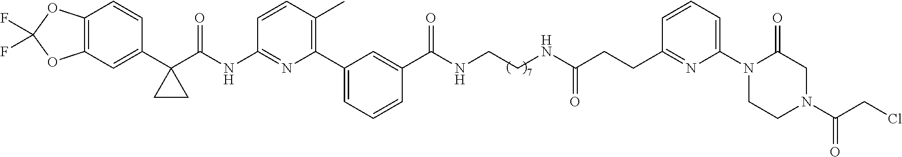 | N-(8-(3-(6-(4-(2- chloroacetyl)-2- oxopiperazin-1- yl)pyridin-2- yl)propanamido)octyl)- 3-(6-(1-(2,2- difluorobenzo[d][1,3] dioxol-5- yl)cyclopropane-1- carboxamido)-3- methylpyridin-2- yl)benzamide |
| 341 | QC179- 109 | 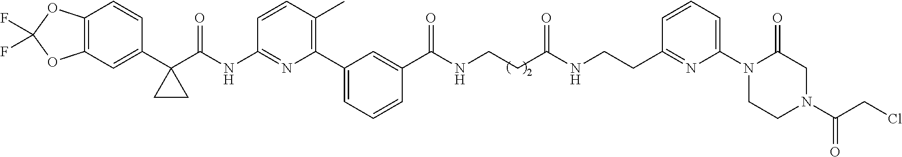 | N-(4-((2-(6-(4-(2- chloroacetyl)-2- oxopiperazin-1- yl)pyridin-2- yl)ethyl)amino)-4- oxobutyl)-3-(6-(1-(2,2- difluorobenzo[d][1,3] dioxol-5- yl)cyclopropane-1- carboxamido)-3- methylpyridin-2- yl)benzamide |
| 342 | QC179- 110 | 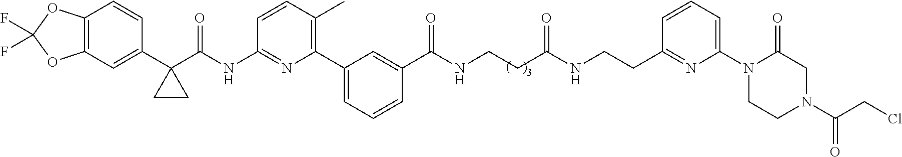 | N-(5-((2-(6-(4-(2- chloroacetyl)-2- oxopiperazin-1- yl)pyridin-2- yl)ethyl)amino)-5- oxopentyl)-3-(6-(1- (2,2- difluorobenzo[d][1,3] dioxol-5- yl)cyclopropane-1- carboxamido)-3- methylpyridin-2- yl)benzamide |
| 343 | QC179- 111 | 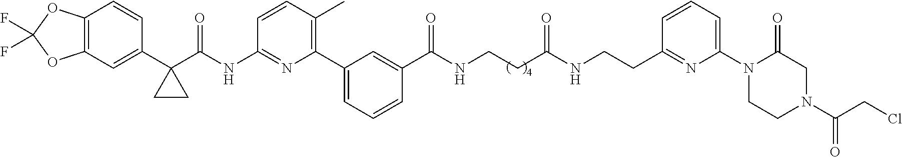 | N-(6-((2-(6-(4-(2- chloroacetyl)-2- oxopiperazin-1- yl)pyridin-2- yl)ethyl)amino)-6- oxohexyl)-3-(6-(1-(2,2- difluorobenzo[d][1,3] dioxol-5- yl)cyclopropane-1- carboxamido)-3- methylpyridin-2- yl)benzamide |
| 344 | QC179- 112 | 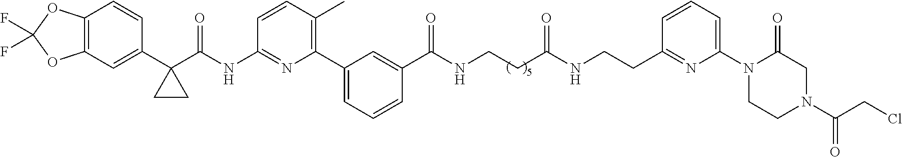 | N-(7-((2-(6-(4-(2- chloroacetyl)-2- oxopiperazin-1- yl)pyridin-2- yl)ethyl)amino)-7- oxoheptyl)-3-(6-(1- (2,2- difluorobenzo[d][1,3] dioxol-5- yl)cyclopropane-1- carboxamido)-3- methylpyridin-2- yl)benzamide |
| 345 | QC179- 113 | 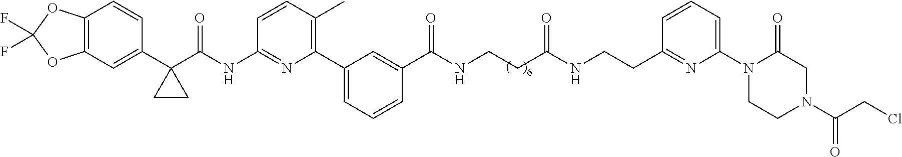 | N-(8-((2-(6-(4-(2- chloroacetyl)-2- oxopiperazin-1- yl)pyridin-2- yl)ethyl)amino)-8- oxooctyl)-3-(6-(1-(2,2- difluorobenzo[d][1,3] dioxol-5- yl)cyclopropane-1- carboxamido)-3- methylpyridin-2- yl)benzamide |
| 346 | QC179- 137 | 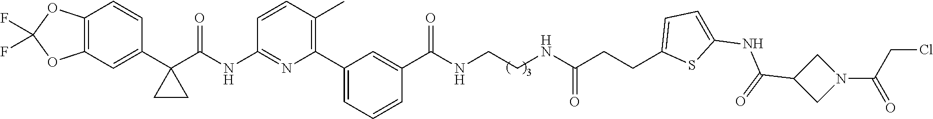 | 1-(2-chloroacetyl)-N- (5-(3-((4-(3-(6-(1-(2,2- difluorobenzo[d][1,3] dioxol-5- yl)cyclopropane-1- carboxamido)-3- methylpyridin-2- yl)benzamido)butyl) amino)-3- oxopropyl)thiophen-2- yl)azetidine-3- carboxamide |
| 347 | QC179- 138 | 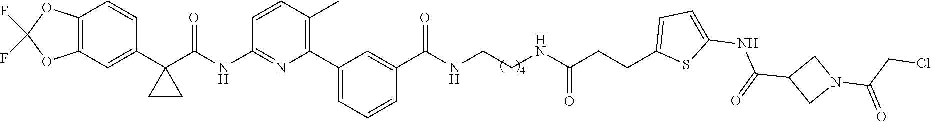 | 1-(2-chloroacetyl)-N- (5-(3-((5-(3-(6-(1-(2,2- difluorobenzo[d][1,3] dioxol-5- yl)cyclopropane-1- carboxamido)-3- methylpyridin-2- yl)benzamido)pentyl) amino)-3- oxopropyl)thiophen-2- yl)azetidine-3- carboxamide |
| 348 | QC179- 139 | 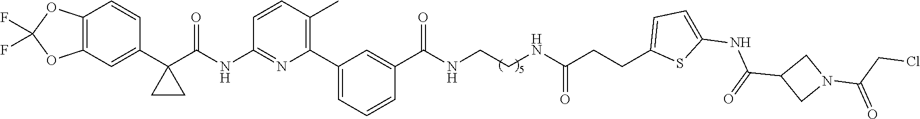 | 1-(2-chloroacetyl)-N- (5-(3-((6-(3-(6-(1-(2,2- difluorobenzo[d][1,3] dioxol-5- yl)cyclopropane-1- carboxamido)-3- methylpyridin-2- yl)benzamido)hexyl) amino)-3- oxopropyl)thiophen-2- yl)azetidine-3- carboxamide |
| 349 | QC179- 140 | 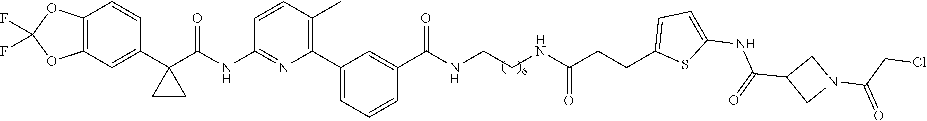 | 1-(2-chloroacetyl)-N- (5-(3-((7-(3-(6-(1-(2,2- difluorobenzo[d][1,3] dioxol-5- yl)cyclopropane-1- carboxamido)-3- methylpyridin-2- yl)benzamido)heptyl) amino)-3- oxopropyl)thiophen-2- yl)azetidine-3- carboxamide |
| 350 | QC179- 141 | 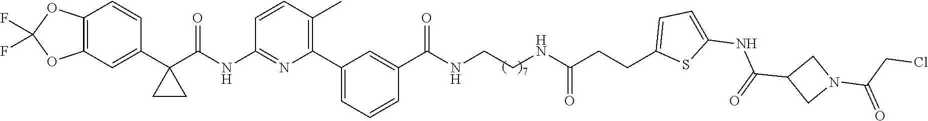 | 1-(2-chloroacetyl)-N- (5-(3-((8-(3-(6-(1-(2,2- difluorobenzo[d][1,3] dioxol-5- yl)cyclopropane-1- carboxamido)-3- methylpyridin-2- yl)benzamido)octyl) amino)-3- oxopropyl)thiophen-2- yl)azetidine-3- carboxamide |
| 351 | QC179- 154 | 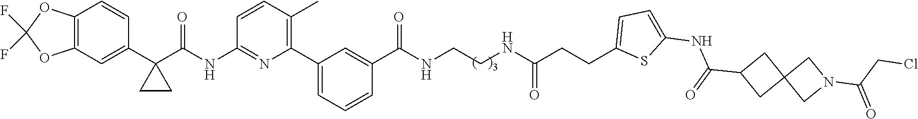 | 2-(2-chloroacetyl)-N- (5-(3-((4-(3-(6-(1-(2,2- difluorobenzo[d][1,3] dioxol-5- yl)cyclopropane-1- carboxamido)-3- methylpyridin-2- yl)benzamido)butyl) amino)-3- oxopropyl)thiophen-2- yI)-2- azaspiro[3.3]heptane- 6-carboxamide |
| 352 | QC179- 155 | 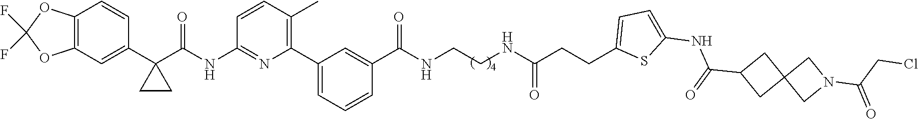 | 2-(2-chloroacetyl)-N- (5-(3-((5-(3-(6-(1-(2,2- difluorobenzo[d][1,3] dioxol-5- yl)cyclopropane-1- carboxamido)-3- methylpyridin-2- yl)benzamido)pentyl) amino)-3- oxopropyl)thiophen-2- yl)-2- azaspiro[3.3]heptane- 6-carboxamide |
| 353 | QC179- 156 | 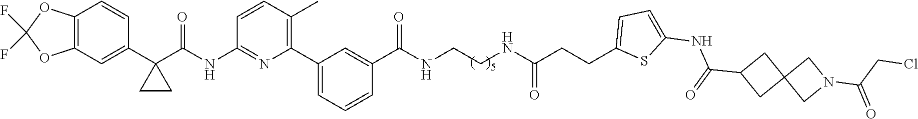 | 2-(2-chloroacetyl)-N- (5-(3-((6-(3-(6-(1-(2,2- difluorobenzo[d][1,3] dioxol-5- yl)cyclopropane-1- carboxamido)-3- methylpyridin-2- yl)benzamido)hexyl) amino)-3- oxopropyl)thiophen-2- yl)-2- azaspiro[3.3]heptane- 6-carboxamide |
| 354 | QC179- 157 | 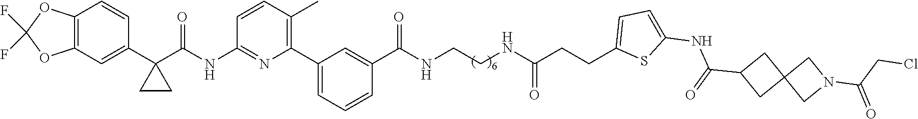 | 2-(2-chloroacetyl)-N- (5-(3-((7-(3-(6-(1-(2,2- difluorobenzo[d][1,3] dioxol-5- yl)cyclopropane-1- carboxamido)-3- methylpyridin-2- yl)benzamido)heptyl) amino)-3- oxopropyl)thiophen-2- yl)-2- azaspiro[3.3]heptane- 6-carboxamide |
| 355 | QC179- 158 | 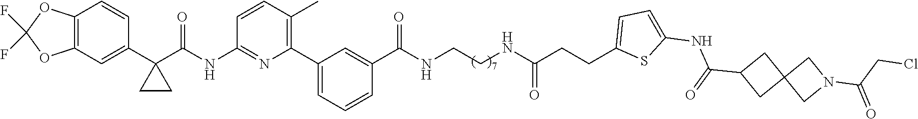 | 2-(2-chloroacetyl)-N- (5-(3-((8-(3-(6-(1-(2,2- difluorobenzo[d][1,3] dioxol-5- yl)cyclopropane-1- carboxamido)-3- methylpyridin-2- yl)benzamido)octyl) amino)-3- oxopropyl)thiophen-2- yl)-2- azaspiro[3.3]heptane- 6-carboxamide |
| 356 | QC192- 153 | 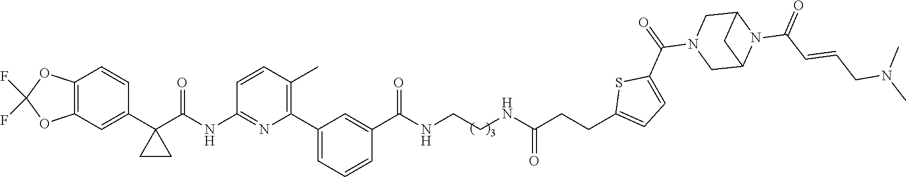 | (E)-3-(6-(1-(2,2- difluorobenzo[d][1,3] dioxol-5- yl)cyclopropane-1- carboxamido)-3- methylpyridin-2-yl)- N-(4-(3-(5-(6-(4- (dimethylamino)but- 2-enoyl)-3,6- diazabicyclo[3.1.1] heptane-3- carbonyl)thiophen-2- yl)propanamido)butyl) benzamide |
| 357 | QC192- 154 | 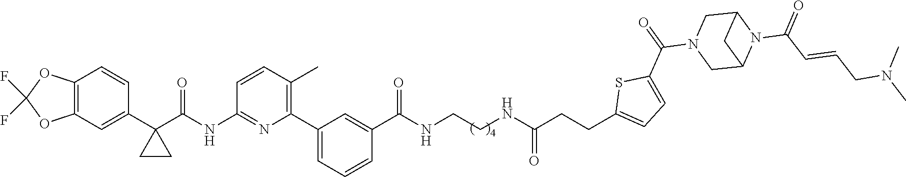 | (E)-3-(6-(1-(2,2- difluorobenzo[d][1,3] dioxol-5- yl)cyclopropane-1- carboxamido)-3- methylpyridin-2-yl)- N-(5-(3-(5-(6-(4- (dimethylamino)but- 2-enoyl)-3,6- diazabicyclo[3.1.1] heptane-3- carbonyl)thiophen-2- yl)propanamido) pentyl)benzamide |
| 358 | QC192- 155 | 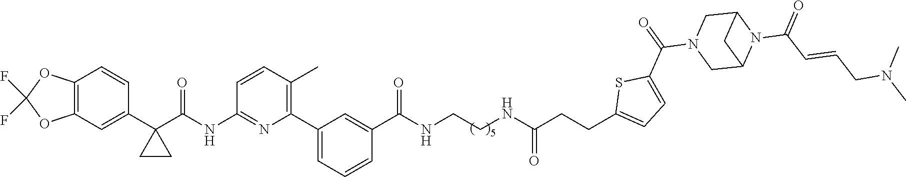 | (E)-3-(6-(1-(2,2- difluorobenzo[d][1,3] dioxol-5- yl)cyclopropane-1- carboxamido)-3- methylpyridin-2-yl)- N-(6-(3-(5-(6-(4- (dimethylamino)but- 2-enoyl)-3,6- diazabicyclo[3.1.1] heptane-3- carbonyl)thiophen-2- yl)propanamido)hexyl) benzamide |
| 359 | QC192- 156 | 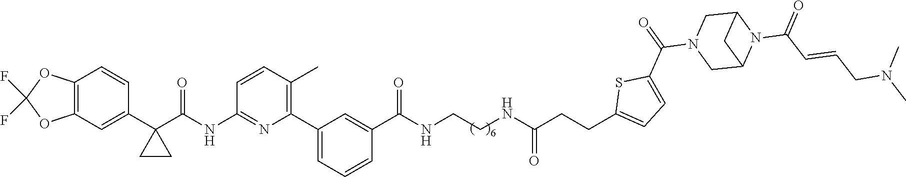 | (E)-3-(6-(1-(2,2- difluorobenzo[d][1,3] dioxol-5- yl)cyclopropane-1- carboxamido)-3- methylpyridin-2-yl)- N-(7-(3-(5-(6-(4- (dimethylamino)but- 2-enoyl)-3,6- diazabicyclo[3.1.1] heptane-3- carbonyl)thiophen-2- yl)propanamido) heptyl)benzamide |
| 360 | QC192- 157 | 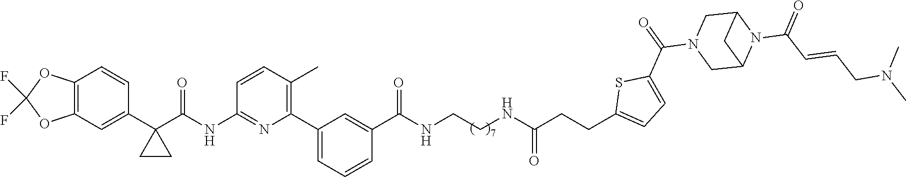 | (E)-3-(6-(1-(2,2- difluorobenzo[d][1,3] dioxol-5- yl)cyclopropane-1- carboxamido)-3- methylpyridin-2-yl)- N-(8-(3-(5-(6-(4- (dimethylamino)but- 2-enoyl)-3,6- diazabicyclo[3.1.1] heptane-3- carbonyl)thiophen-2- yl)propanamido)octyl) benzamide |
| 361 | QC192- 184 | 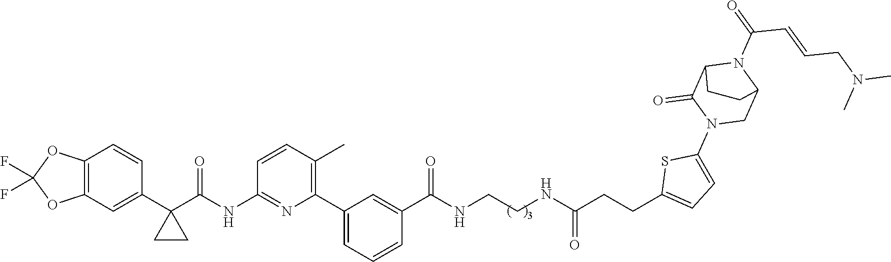 | (E)-3-(6-(1-(2,2- difluorobenzo[d][1,3] dioxol-5- yl)cyclopropane-1- carboxamido)-3- methylpyridin-2-yl)- N-(4-(3-(5-(8-(4- (dimethylamino)but- 2-enoyl)-2-oxo-3,8- diazabicyclo[3.2.1] octan-3-yl)thiophen-2- yl)propanamido)butyl) benzamide |
| 362 | QC192- 178 | 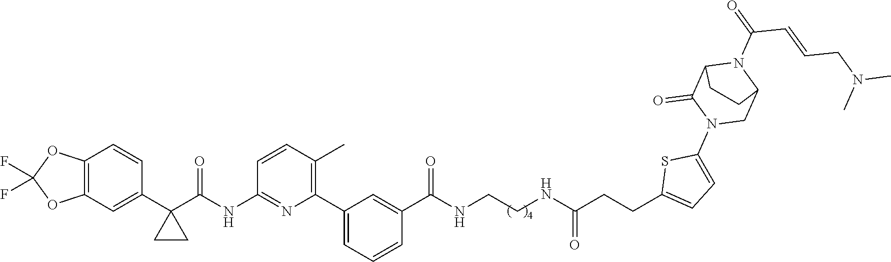 | (E)-3-(6-(1-(2,2- difluorobenzo[d][1,3] dioxol-5- yl)cyclopropane-1- carboxamido)-3- methylpyridin-2-yl)- N-(5-(3-(5-(8-(4- (dimethylamino)but- 2-enoyl)-2-oxo-3,8- diazabicyclo[3.2.1] octan-3-yl)thiophen-2- yl)propanamido) pentyl)benzamide |
| 363 | QC192- 179 | 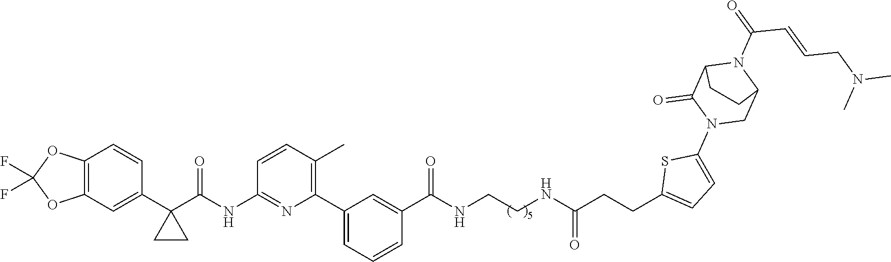 | (E)-3-(6-(1-(2,2- difluorobenzo[d][1,3] dioxol-5- yl)cyclopropane-1- carboxamido)-3- methylpyridin-2-yl)- N-(6-(3-(5-(8-(4- (dimethylamino)but- 2-enoyl)-2-oxo-3,8- diazabicyclo[3.2.1] octan-3-yl)thiophen-2- yl)propanamido)hexyl) benzamide |
| 364 | QC192- 180 | 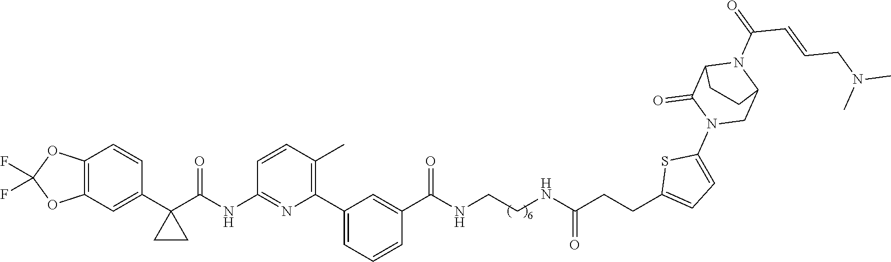 | (E)-3-(6-(1-(2,2- difluorobenzo[d][1,3] dioxol-5- yl)cyclopropane-1- carboxamido)-3- methylpyridin-2-yl)- N-(7-(3-(5-(8-(4- (dimethylamino)but- 2-enoyl)-2-oxo-3,8- diazabicyclo[3.2.1] octan-3-yl)thiophen-2- yl)propanamido)heptyl) benzamide |
| 365 | QC192- 181 | 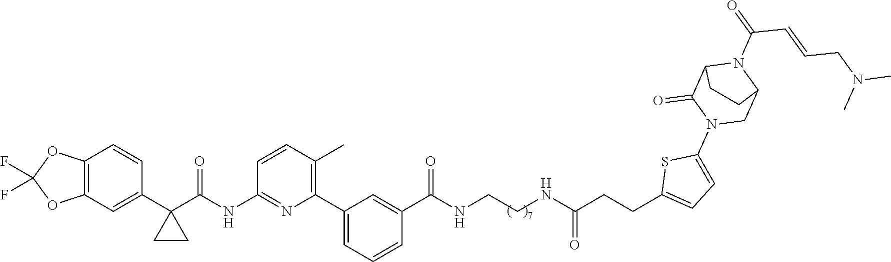 | (E)-3-(6-(1-(2,2- difluorobenzo[d][1,3] dioxol-5- yl)cyclopropane-1- carboxamido)-3- methylpyridin-2-yl)- N-(8-(3-(5-(8-(4- (dimethylamino)but- 2-enoyl)-2-oxo-3,8- diazabicyclo[3.2.1] octan-3-yl)thiophen-2- yl)propanamido)octyl) benzamide |
| 366 | QC192- 182 | 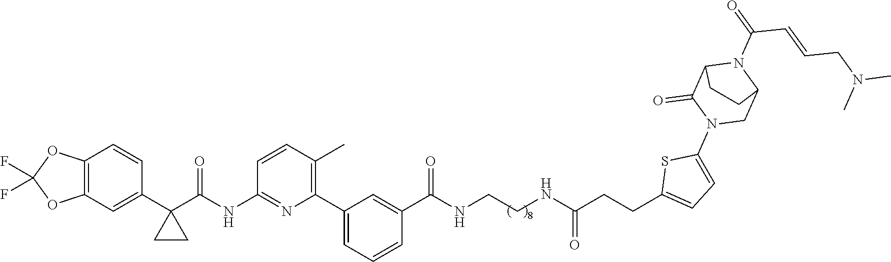 | (E)-3-(6-(1-(2,2- difluorobenzo[d][1,3] dioxol-5- yl)cyclopropane-1- carboxamido)-3- methylpyridin-2-yl)- N-(9-(3-(5-(8-(4- (dimethylamino)but- 2-enoyl)-2-oxo-3,8- diazabicyclo[3.2.1] octan-3-yl)thiophen-2- yl)propanamido)nonyl) benzamide |

Additional OTUB1 recruiting bivalent compounds that can be made are shown in table 5.

TABLE 5

| Example No. | Structure | Name |
|---|---|---|
| 367 | 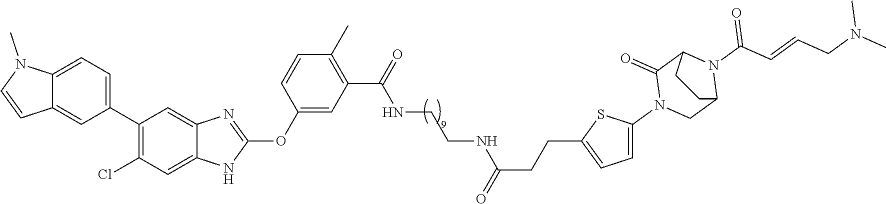 | (E)-N-(2-((5-((6-chloro-5- (1-methyl-1H-indol-5-yl)- 1H-benzo[d]imidazol-2- yl)oxy)-2- methylbenzoyl)octamethyl- λ¹¹-azaneyl)ethyl)-3-(5- (8-(4-(dimethylamino)but- 2-enoyl)-2-oxo-3,8- diazabicyclo[3.2.1]octan- 3-yl)thiophen-2- yl)propanamide |
| 368 | 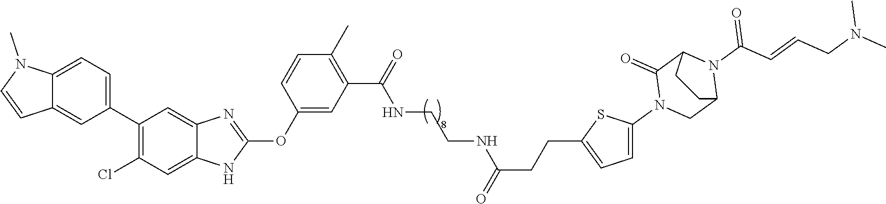 | (E)-N-(2-((5-((6-chloro-5- (1-methyl-1H-indol-5-yl)- 1H-benzo[d]imidazol-2- yl)oxy)-2- methylbenzoyl)heptamethyl- λ¹⁰-azaneyl)ethyl)-3-(5- (8-(4-(dimethylamino)but- 2-enoyl)-2-oxo-3,8- diazabicyclo[3.2.1]octan- 3-yl)thiophen-2- yl)propanamide |
| 369 | 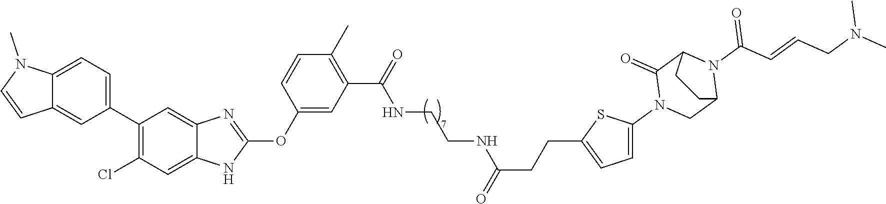 | (E)-N-(2-((5-((6-chloro-5- (1-methyl-1H-indol-5-yl)- 1H-benzo[d]imidazol-2- yl)oxy)-2- methylbenzoyl)hexamethyl- λ⁹-azaneyl)ethyl)-3-(5- (8-(4-(dimethylamino)but- 2-enoyl)-2-oxo-3,8- diazabicyclo[3.2.1]octan- 3-yl)thiophen-2- yl)propanamide |
| 370 | 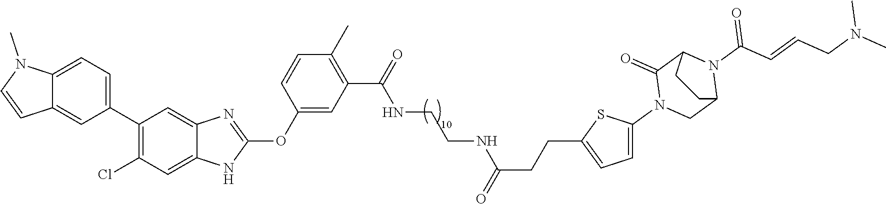 | (E)-N-(2-((5-((6-chloro-5- (1-methyl-1H-indol-5-yl)- 1H-benzo[d]imidazol-2- yl)oxy)-2- methylbenzoyl)nonamethyl- λ¹²-azaneyl)ethyl)-3-(5- (8-(4-(dimethylamino)but- 2-enoyl)-2-oxo-3,8- diazabicyclo[3.2.1]octan- 3-yl)thiophen-2- yl)propanamide |
| 371 | 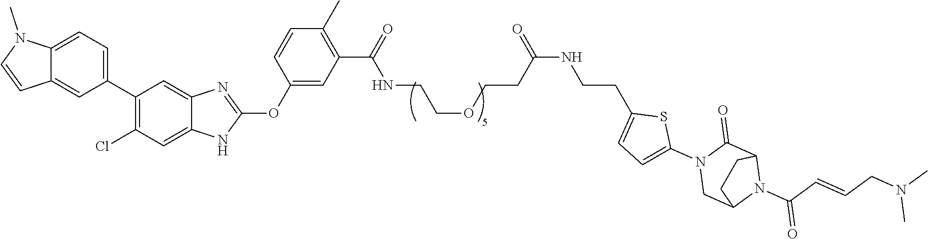 | (E)-1-(5-((6-chloro-5-(1- methyl-1H-indol-5-yl)-1H- benzo[d]imidazol-2- yl)oxy)-2- methylbenzamido)-N-(2- (5-(8-(4- (dimethylamino)but-2- enoyl)-2-oxo-3,8- diazabicyclo[3.2.1]octan- 3-yl)thiophen-2-yl)ethyl)- 3,6,9,12,15- pentaoxaoctadecan-18- amide |
| 372 | 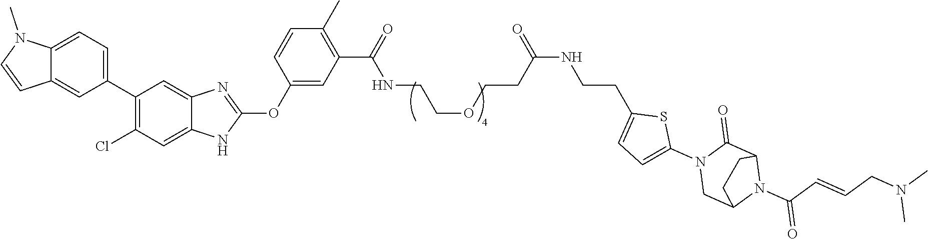 | (E)-1-(5-((6-chloro-5-(1- methyl-1H-indol-5-yl)-1H- benzo[d]imidazol-2- yl)oxy)-2- methylbenzamido)-N-(2- (5-(8-(4- (dimethylamino)but-2- enoyl)-2-oxo-3,8- diazabicyclo[3.2.1]octan- 3-yl)thiophen-2-yl)ethyl)- 3,6,9,12- tetraoxapentadecan-15- amide |
| 373 | 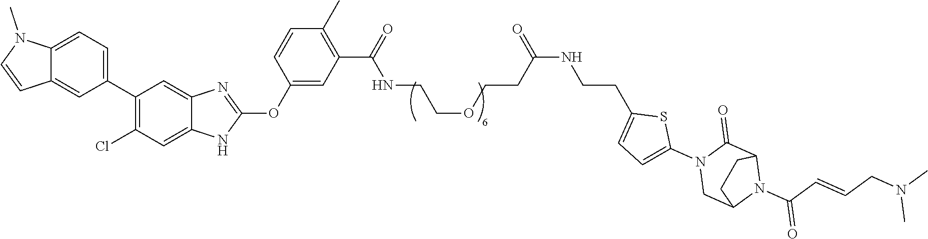 | (E)-1-(5-((6-chloro-5-(1- methyl-1H-indol-5-yl)-1H- benzo[d]imidazol-2- yl)oxy)-2- methylbenzamido)-N-(2- (5-(8-(4- (dimethylamino)but-2- enoyl)-2-oxo-3,8- diazabicyclo[3.2.1]octan- 3-yl)thiophen-2-yl)ethyl)- 3,6,9,12,15,18- hexaoxahenicosan-21- amide |
| 374 | 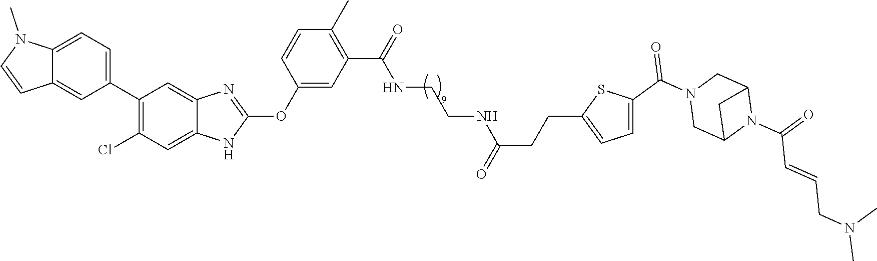 | (E)-N-(2-((5-((6-chloro-5- (1-methyl-1H-indol-5-yl)- 1H-benzo[d]imidazol-2- yl)oxy)-2- methylbenzoyl)octamethyl- λ¹¹-azaneyl)ethyl)-3-(5- (6-(4-(dimethylamino)but- 2-enoyl)-3,6- diazabicyclo[3.1.1]heptane- 3-carbonyl)thiophen-2- yl)propanamide |
| 375 | 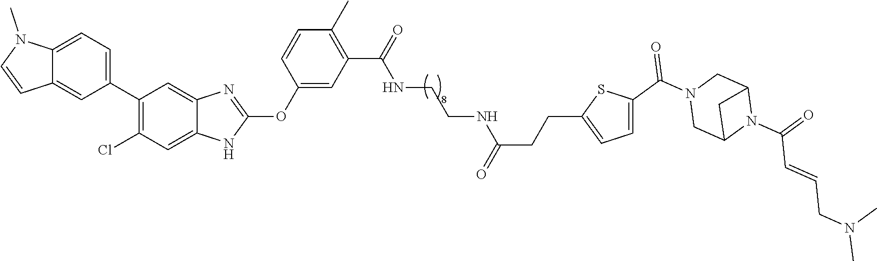 | (E)-N-(2-((5-((6-chloro-5- (1-methyl-1H-indol-5-yl)- 1H-benzo[d]imidazol-2- yl)oxy)-2- methylbenzoyl)heptamethyl- λ¹⁰-azaneyl)ethyl)-3-(5- (6-(4-(dimethylamino)but- 2-enoyl)-3,6- diazabicyclo[3.1.1]heptane- 3-carbonyl)thiophen-2- yl)propanamide |
| 376 | 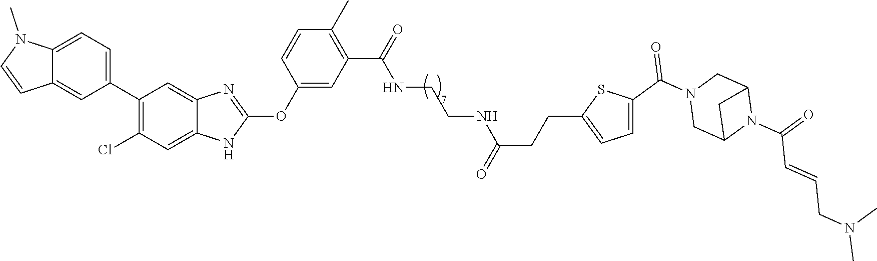 | (E)-N-(2-((5-((6-chloro-5- (1-methyl-1H-indol-5-yl)- 1H-benzo[d]imidazol-2- yl)oxy)-2- methylbenzoyl)hexamethyl- λ⁹-azaneyl)ethyl)-3-(5- (6-(4-(dimethylamino)but- 2-enoyl)-3,6- diazabicyclo[3.1.1]heptane- 3-carbonyl)thiophen-2- yl)propanamide |
| 377 | 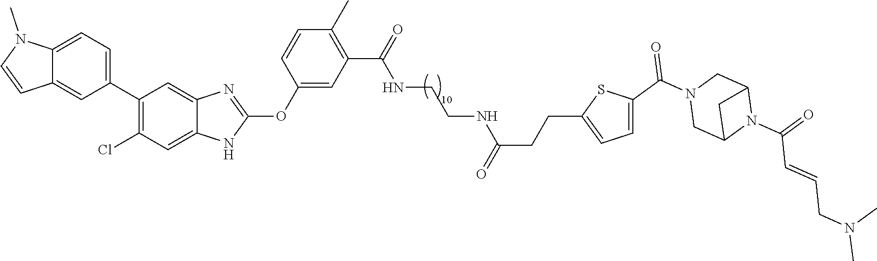 | (E)-N-(2-((5-((6-chloro-5- (1-methyl-1H-indol-5-yl)- 1H-benzo[d]imidazol-2- yl)oxy)-2- methylbenzoyl)nonamethyl- λ¹²-azaneyl)ethyl)-3-(5- (6-(4-(dimethylamino)but- 2-enoyl)-3,6- diazabicyclo[3.1.1]heptane- 3-carbonyl)thiophen-2- yl)propanamide |
| 378 | 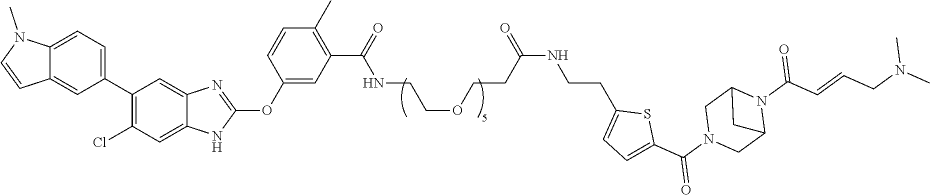 | (E)-1-(5-((6-chloro-5-(1- methyl-1H-indol-5-yl)-1H- benzo[d]imidazol-2- yl)oxy)-2- methylbenzamido)-N-(2- (5-(6-(4- (dimethylamino)but-2- enoyl)-3,6- diazabicyclo[3.1.1]heptane- 3-carbonyl)thiophen-2- yl)ethyl)-3,6,9,12,15- pentaoxaoctadecan-18- amide |
| 379 | 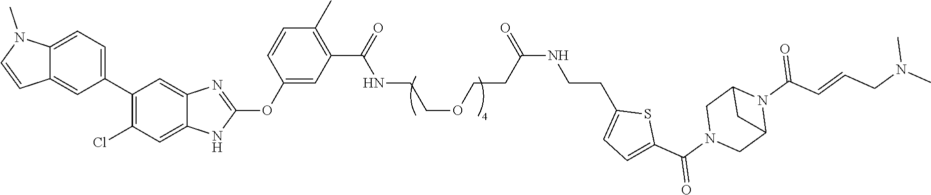 | (E)-1-(5-((6-chloro-5-(1- methyl-1H-indol-5-yl)-1H- benzo[d]imidazol-2- yl)oxy)-2- methylbenzamido)-N-(2- (5-(6-(4- (dimethylamino)but-2- enoyl)-3,6- diazabicyclo[3.1.1]heptane- 3-carbonyl)thiophen-2- yl)ethyl)-3,6,9,12- tetraoxapentadecan-15- amide |
| 380 | 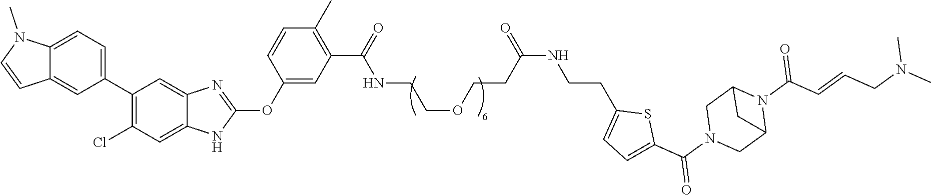 | (E)-1-(5-((6-chloro-5-(1- methyl-1H-indol-5-yl)-1H- benzo[d]imidazol-2- yl)oxy)-2- methylbenzamido)-N-(2- (5-(6-(4- (dimethylamino)but-2- enoyl)-3,6- diazabicyclo[3.1.1]heptane- 3-carbonyl)thiophen-2- yl)ethyl)-3,6,9,12,15,18- hexaoxahenicosan-21- amide |
| 381 | 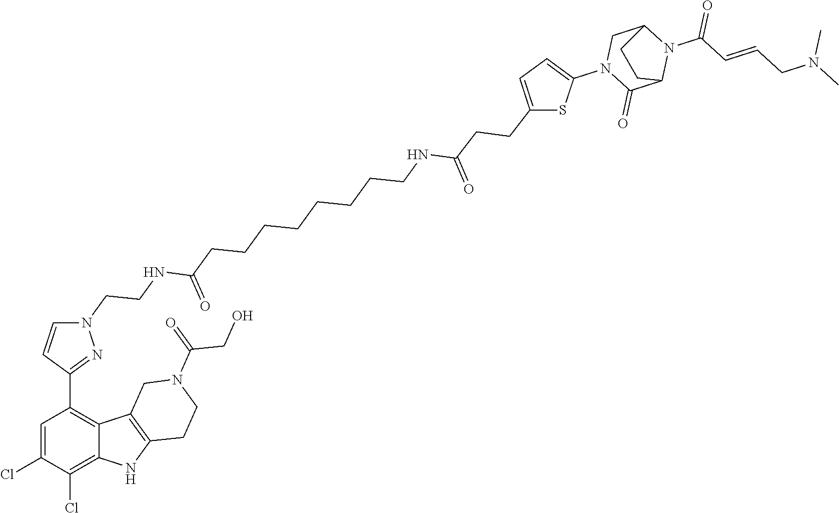 [SMILES: CN(C)C/C=C/C(=O)N1C2CCC1C(=O)N(c1ccc(CCC(=O)NCCCCCCCCC(=O)NCCn3ccc(-c4cc(Cl)c(Cl)c5[nH]c6c(c45)CN(C(=O)CO)CC6)n3)s1)C2] | (E)-N-(2-(3-(6,7-dichloro- 2-(2-hydroxyacetyl)- 2,3,4,5-tetrahydro-1H- pyrido[4,3-b]indol-9-yl)- 1H-pyrazol-1-yl)ethyl)-9- (3-(5-(8-(4- (dimethylamino)but-2- enoyl)-2-oxo-3,8- diazabicyclo[3.2.1]octan- 3-yl)thiophen-2- yl)propanamido) nonanamide |
| 382 | 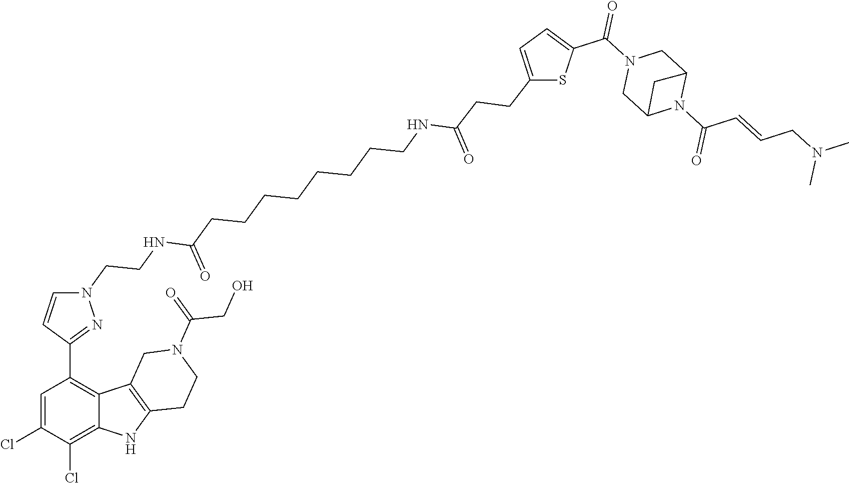 [SMILES: CN(C)C/C=C/C(=O)N1C2CC1CN(C(=O)c1ccc(CCC(=O)NCCCCCCCCC(=O)NCCn3ccc(-c4cc(Cl)c(Cl)c5[nH]c6c(c45)CN(C(=O)CO)CC6)n3)s1)C2] | (E)-N-(2-(3-(6,7-dichloro- 2-(2-hydroxyacetyl)- 2,3,4,5-tetrahydro-1H- pyrido[4,3-b]indol-9-yl)- 1H-pyrazol-1-yl)ethyl)-9- (3-(5-(6-(4- (dimethylamino)but-2- enoyl)-3,6- diazabicyclo[3.1.1]heptane- 3-carbonyl)thiophen-2- yl)propanamido) nonanamide |
| 383 | 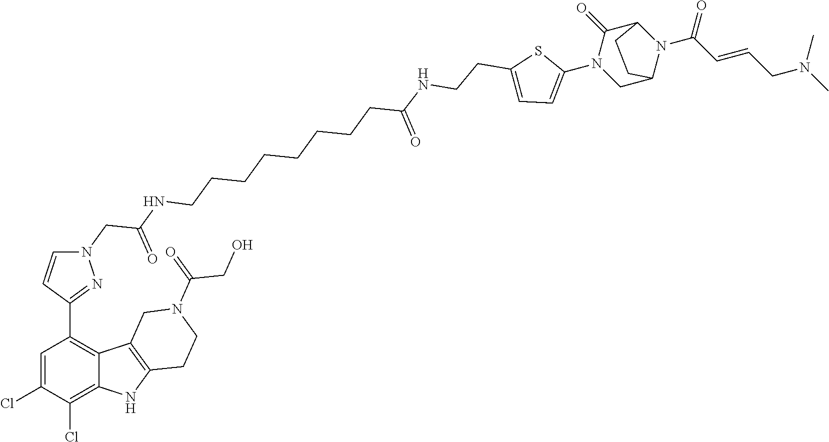 [SMILES: CN(C)C/C=C/C(=O)N1C2CCC1C(=O)N(c1ccc(CCNC(=O)CCCCCCCCNC(=O)Cn3ccc(-c4cc(Cl)c(Cl)c5[nH]c6c(c45)CN(C(=O)CO)CC6)n3)s1)C2] | (E)-9-(2-(3-(6,7-dichloro- 2-(2-hydroxyacetyl)- 2,3,4,5-tetrahydro-1H- pyrido[4,3-b]indol-9-yl)- 1H-pyrazol-1- yl)acetamido)-N-(2-(5-(8- (4-(dimethylamino)but-2- enoyl)-2-oxo-3,8- diazabicyclo[3.2.1]octan- 3-yl)thiophen-2- yl)ethyl)nonanamide |
| 384 | 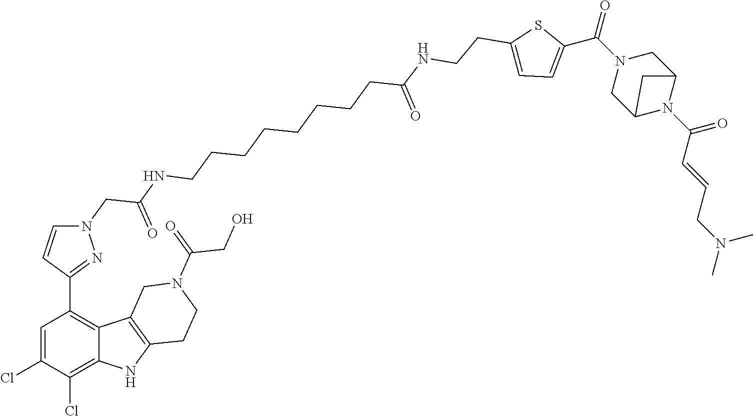 [SMILES: CN(C)C/C=C/C(=O)N1C2CC1CN(C(=O)c1ccc(CCNC(=O)CCCCCCCCNC(=O)Cn3ccc(-c4cc(Cl)c(Cl)c5[nH]c6c(c45)CN(C(=O)CO)CC6)n3)s1)C2] | (E)-9-(2-(3-(6,7-dichloro- 2-(2-hydroxyacetyl)- 2,3,4,5-tetrahydro-1H- pyrido[4,3-b]indol-9-yl)- 1H-pyrazol-1- yl)acetamido)-N-(2-(5-(6- (4-(dimethylamino)but-2- enoyl)-3,6- diazabicyclo[3.1.1]heptane- 3-carbonyl)thiophen-2- yl)ethyl)nonanamide |
| 385 | 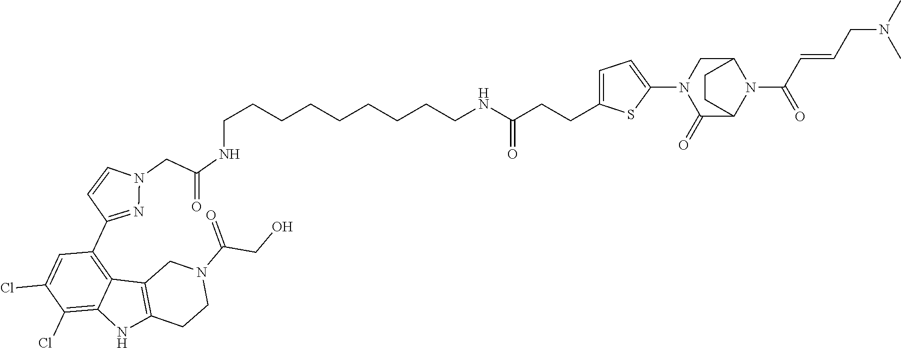 [SMILES: CN(C)C/C=C/C(=O)N1C2CCC1C(=O)N(c1ccc(CCC(=O)NCCCCCCCCCNC(=O)Cn3ccc(-c4cc(Cl)c(Cl)c5[nH]c6c(c45)CN(C(=O)CO)CC6)n3)s1)C2] | (E)-N-(9-(2-(3-(6,7- dichloro-2-(2- hydroxyacetyl)-2,3,4,5- tetrahydro-1H-pyrido[4,3- b]indol-9-yl)-1H-pyrazol- 1-yl)acetamido)nonyl)-3- (5-(8-(4- (dimethylamino)but-2- enoyl)-2-oxo-3,8- diazabicyclo[3.2.1]octan- 3-yl)thiophen-2- yl)propanamide |
| 386 | 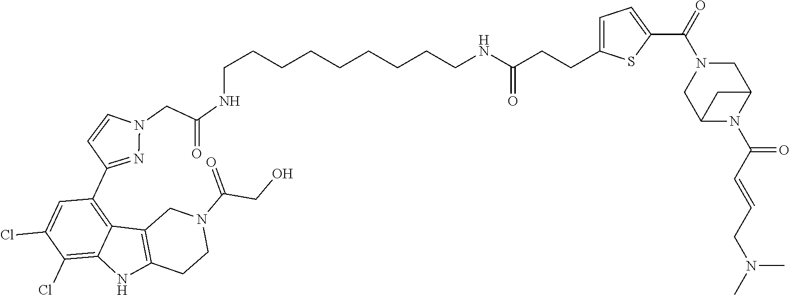 [SMILES: CN(C)C/C=C/C(=O)N1C2CC1CN(C(=O)c1ccc(CCC(=O)NCCCCCCCCCNC(=O)Cn3ccc(-c4cc(Cl)c(Cl)c5[nH]c6c(c45)CN(C(=O)CO)CC6)n3)s1)C2] | (E)-N-(9-(2-(3-(6,7- dichloro-2-(2- hydroxyacetyl)-2,3,4,5- tetrahydro-1H-pyrido[4,3 b]indol-9-yl)-1H-pyrazol- 1-yl)acetamido)nonyl)-3- (5-(6-(4- (dimethylamino)but-2- enoyl)-3,6- diazabicyclo[3.1.1]heptane- 3-carbonyl)thiophen-2- yl)propanamide |
| 387 | 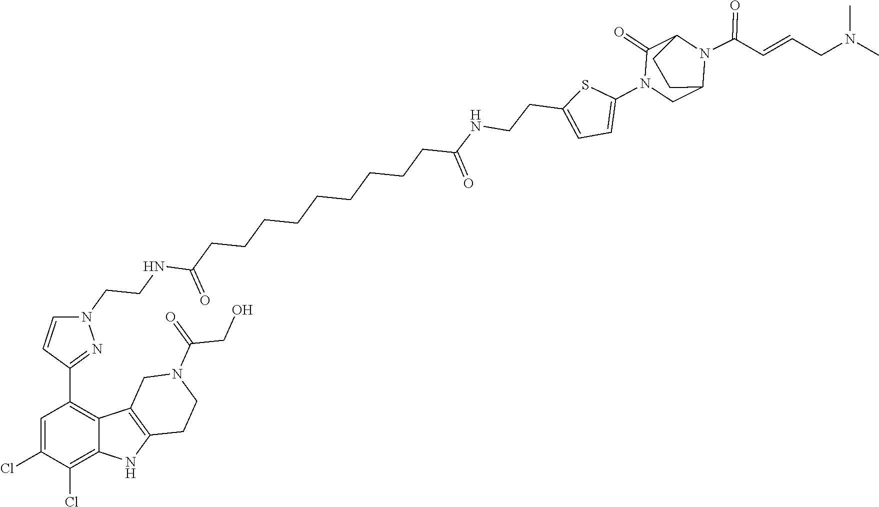 [SMILES: CN(C)C/C=C/C(=O)N1C2CCC1C(=O)N(c1ccc(CCNC(=O)CCCCCCCCCC(=O)NCCn3ccc(-c4cc(Cl)c(Cl)c5[nH]c6c(c45)CN(C(=O)CO)CC6)n3)s1)C2] | (E)-N¹-(2-(3-(6,7-dichloro- 2-(2-hydroxyacetyl)- 2,3,4,5-tetrahydro-1H- pyrido[4,3-b]indol-9-yl)- 1H-pyrazol-1-yl)ethyl)- N¹¹-(2-(5-(8-(4- (dimethylamino)but-2- enoyl)-2-oxo-3,8- diazabicyclo[3.2.1]octan- 3-yl)thiophen-2- yl)ethyl)undecanediamide |
| 388 | 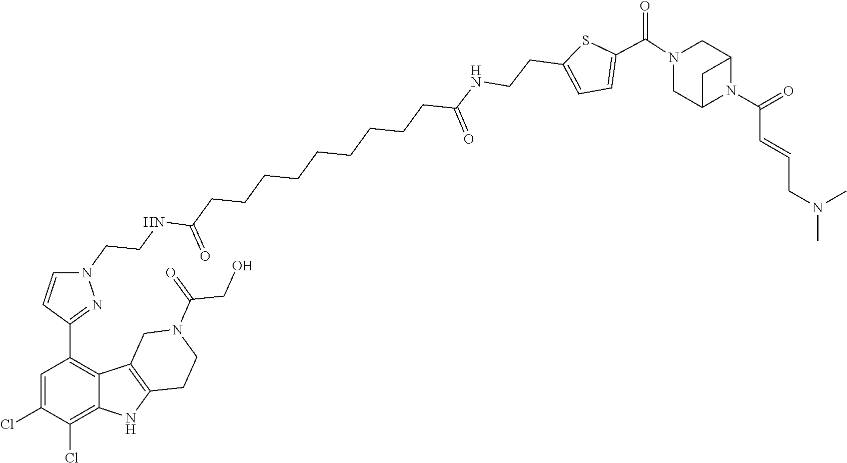 [SMILES: CN(C)C/C=C/C(=O)N1C2CC1CN(C(=O)c1ccc(CCNC(=O)CCCCCCCCCC(=O)NCCn3ccc(-c4cc(Cl)c(Cl)c5[nH]c6c(c45)CN(C(=O)CO)CC6)n3)s1)C2] | (E)-N¹-(2-(3-(6,7-dichloro- 2-(2-hydroxyacetyl)- 2,3,4,5-tetrahydro-1H- pyrido[4,3-b]indol-9-yl)- 1H-pyrazol-1-yl)ethyl)- N¹¹-(2-(5-(6-(4- (dimethylamino)but-2- enoyl)-3,6- diazabicyclo[3.1.1]heptane- 3-carbonyl)thiophen-2- yl)ethyl)undecanediamide |

As used herein, in case of discrepancy between the structure and chemical name provided for a particular compound, the structure shall control.

OTUB1 covalent binders were tested for their ability to form the covalent adduct with OTUB1 protein using a mass-spectrometry based assay.

TABLE 6

| Example | Compound | % of OTUB1 | Example | Compound | % of OTUB1 |
|---|---|---|---|---|---|
| number | ID | modified | number | ID | modified |
| | EN523 | 40% | 1 | XS154-91 | 52% |
| 2 | XS154-130 | N/A | 3 | XS154-148 | 39% |
| 4 | XS154-114 | N/A | 5 | XS154-149 | N/A |
| 6 | XS159-13 | N/A | 7 | XS159-107 | N/A |
| 8 | XS165-30 | <5% | 9 | XS159-90 | <5% |
| 10 | XS165-53 | 5% | 11 | XS165-38 | N/A |
| 12 | XS165-33 | 5% | 13 | XS165-54 | 1% |
| 14 | XS154-184 | <5% | 15 | XS165-75 | N/A |
| 16 | XS165-77 | 100% | 17 | XS165-127 | N/A |
| 18 | XS165-106 | N/A | 19 | XS165-112 | N/A |
| 20 | XS165-97 | N/A | 21 | XS165-118 | N/A |
| 22 | XS165-110 | N/A | 23 | XS165-119 | 25% |
| 24 | XS165-123 | 100% | 25 | XS165-126 | N/A |
| 26 | XS165-120 | 100% | 27 | XS165-121 | 100% |
| 28 | XS165-100 | 25% | 29 | XS165-99 | 38% |
| 30 | XS165-113 | N/A | 31 | XS165-109 | 10% |
| 32 | XS165-117 | N/A | 33 | XS165-105 | 70% |
| 34 | XS165-154 | 70% | 35 | XS165-170 | 70% |
| 36 | XS165-155 | <5% | 37 | XS165-172 | <1% |
| 38 | XS165-169 | <1% | 39 | XS165-177 | 10% |
| 40 | XS175-45 | 45% | 41 | XS175-46 | 90% |
| 42 | XS175-59 | 80% | 43 | XS175-63 | 75% |
| 44 | XS175-64 | 95% | 45 | XS175-67 | 60% |
| 46 | XS175-68 | 70% | 47 | XS175-70 | 90% |
| 48 | XS175-71 | 40% | 49 | XS175-76 | N/A |
| 50 | XS175-110 | N/A | 51 | XS175-120 | 47% |
| 52 | XS175-126 | 100% | 53 | XS175-132 | N/A |
| 54 | XS175-133 | 2% | 55 | XS175-137 | 100% |
| 56 | XS175-143 | 45% | 57 | XS175-148 | 20% |
| 58 | XS175-149 | 18% | 59 | XS175-158 | 45% |
| 60 | XS175-159 | N/A | 61 | XS175-160 | <1% |
| 62 | XS175-173 | 3% | 63 | XS175-174 | N/A |
| 64 | XS175-175 | N/A | 65 | XS175-176 | N/A |
| 66 | XS175-178 | 3% | 67 | XS175-179 | 100% |
| 68 | XS175-180 | 8% | 69 | XS175-186 | 100% |
| 70 | XS185-3 | 35% | 71 | XS185-4 | 100% |
| 72 | XS185-5 | 100% | 73 | XS185-6 | 100% |
| 74 | XS185-24 | 60% | 75 | XS185-29 | N/A |
| 76 | XS185-43 | 58% | 77 | XS185-46 | 1% |
| 78 | XS185-59 | N/A | 79 | XS185-64 | 20% |
| 80 | XS185-65 | 1% | 81 | XS185-66 | 20% |
| 82 | XS185-67 | 15% | 83 | XS185-69 | 30% |
| 84 | XS185-70 | 20% | 85 | XS185-71 | 15% |
| 86 | XS185-72 | 90% | 87 | XS185-75 | 98% |
| 88 | XS185-78 | 40% | 89 | XS185-86 | 80% |
| 90 | XS185-87 | 1% | 91 | XS185-90 | 60% |
| 92 | XS185-91 | 25% | 93 | XS185-96 | 10% |
| 94 | XS185-97 | 25% | 95 | XS185-101 | 23% |
| 96 | XS185-109 | 1% | 97 | XS185-113 | 60% |
| 98 | XS185-114 | 18% | 99 | XS185-116 | 55% |
| 100 | XS185-118 | N/A | 101 | XS185-122 | N/A |
| 102 | XS185-131 | 18% | 103 | XS185-134 | N/A |
| 104 | XS185-135 | N/A | 105 | XS185-138 | N/A |
| 106 | XS185-140 | N/A | 107 | XS185-147 | 15% |
| 108 | XS185-149 | N/A | 109 | XS185-150 | N/A |
| 110 | XS185-171 | N/A | 111 | XS190-9 | 38% |
| 112 | XS190-27 | N/A | 113 | XS190-38 | 100% |
| 114 | XS190-44 | 97% | 115 | XS190-45 | 90% |
| 116 | XS190-46 | 90% | 117 | XS190-47 | 70% |
| 118 | XS190-48 | 90% | 119 | XS190-49 | 70% |
| 120 | XS190-50 | 92% | 121 | XS190-59 | 92% |
| 122 | XS190-68 | N/A | 123 | XS190-69 | 88% |
| 124 | XS190-70 | 97% | 125 | XS190-75 | N/A |
| 126 | XS190-75-2 | N/A | 127 | XS190-77 | 7% |
| 128 | XS190-80 | 7% | 129 | XS190-81 | 10% |
| 130 | XS190-126 | 49% | 131 | XS190-127 | 15% |
| 132 | XS190-128 | 47% | 133 | XS190-129 | 48% |
| 134 | XS190-130 | 53% | 135 | XS190-137 | 20% |
| 136 | XS190-138 | 45% | 137 | XS190-144 | 80% |
| 138 | XS190-157 | 80% | 139 | XS190-158 | 8% |
| 140 | XS190-170 | 55% | 141 | XS190-181 | 30% |
| 142 | XS190-182 | 90% | 143 | XS190-183 | 95% |
| 144 | XS190-186 | 70% | 145 | XS197-3 | N/A |
| 146 | XS197-4 | 75% | 147 | XS197-5 | 25% |
| 148 | XS197-6 | 22% | 149 | XS197-14 | 85% |
| 150 | XS197-29 | 75% | 151 | XS197-38 | 43% |
| 152 | XS197-49 | 85% | 153 | XS197-50 | 8% |
| 154 | XS197-70 | 75% | 155 | XS197-71 | 78% |
| 156 | XS197-72 | 90% | 157 | XS197-73 | 90% |
| 158 | XS197-74 | 92% | 159 | XS197-85 | 45% |
| 160 | XS197-93 | 80% | 161 | XS197-94 | 80% |
| 162 | XS197-96 | 45% | 163 | XS197-133 | 25% |
| 164 | XS197-145 | 90% | 165 | XS197-176 | 30% |
| 166 | XS197-185 | 95% | 167 | XS197-186 | 15% |
| 168 | XS209-21 | 22% | 169 | XS209-22 | 47% |
| 170 | XS209-23 | 10% | 171 | XS209-24 | N/A |
| 172 | XS209-25 | 5% | 173 | XS209-26 | 98% |
| 174 | XS209-27 | N/A | 175 | XS209-28 | 25% |
| 176 | XS209-30 | 75% | 177 | XS209-38 | 20% |
| 178 | XS209-39 | 40% | 179 | XS209-40 | 60% |
| 180 | XS209-53 | 40% | 181 | XS209-54 | 35% |
| 182 | XS209-55 | N/A | 183 | XS209-57 | N/A |
| 184 | XS209-60 | 75% | 185 | XS209-62 | N/A |
| 186 | XS209-65 | 80% | 187 | XS209-74 | 98% |
| 188 | XS209-75 | 75% | 189 | XS209-92 | 15% |
| 190 | XS209-99 | 98% | 191 | XS209-100 | 25% |
| 192 | XS209-101 | 100% | 193 | XS209-122 | 2% |
| 194 | XS209-139 | 2% | 195 | XS209-140 | N/A |
| 196 | XS209-165 | 20% | 197 | XS209-166 | 90% |
| 198 | XS209-167 | N/A | 199 | XS209-168 | 15% |
| 200 | XS209-174 | 70% | 201 | XS209-175 | 90% |
| 202 | XS209-176 | 55% | 203 | XS209-184 | 80% |
| 204 | XS209-185 | 85% | 205 | XS224-6 | N/A |
| 206 | XS224-106 | 98% | 207 | XS224-107 | 60% |
| 208 | XS224-108 | 100% | 209 | XS224-109 | 75% |
| 210 | XS224-110 | 100% | 211 | XS224-111 | 53% |
| 212 | XS224-116 | 100% | 213 | XS224-117 | 60% |
| 214 | XS224-118 | 10% | 215 | XS224-119 | 60% |
| 216 | XS224-143 | 7% | 217 | XS224-144 | 90% |
| 218 | XS224-145 | 60% | 219 | XS224-147 | N/A |
| 220 | XS224-148 | 90% | 221 | XS224-149 | 15% |
| 222 | XS224-150 | 73% | 223 | XS224-154 | 80% |
| 224 | XS224-155 | 97% | 225 | XS224-156 | 85% |
| 226 | XS224-157 | 49% | 227 | XS224-158 | 92% |
| 228 | XS224-159 | 92% | | | |

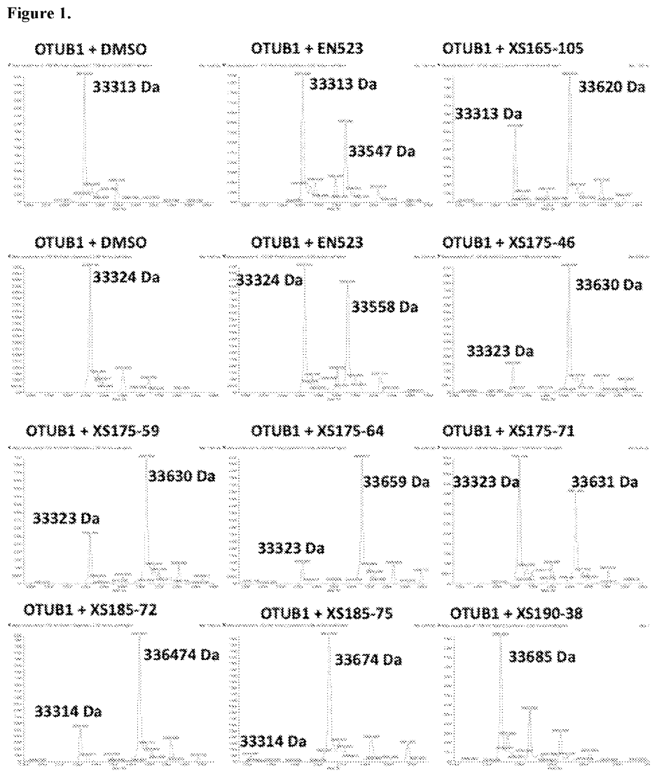

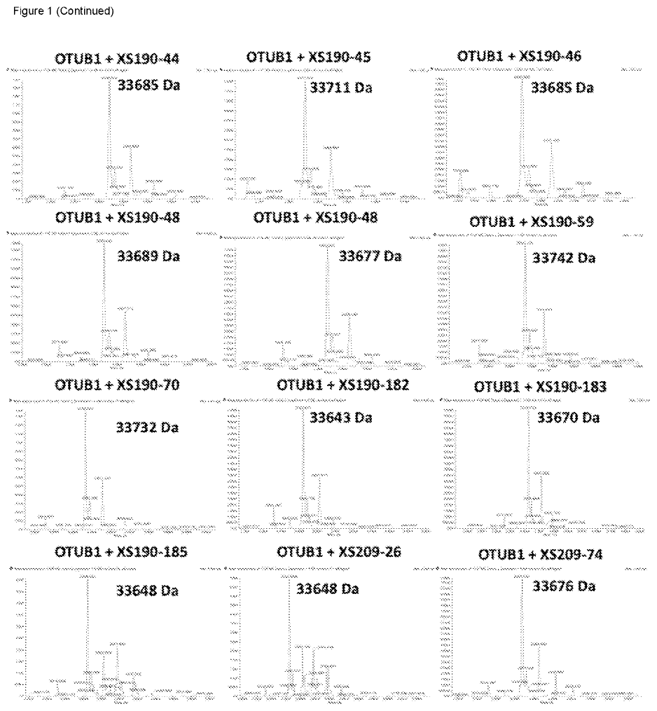

### Example 389. Assessment of the Covalent Modification of OTUB1 Protein by Selected OTUB1 Covalent Binders Using a Mass Spectrometry-Based Assay (FIG. 1)

OTUB1 protein (10 μM) was incubated with selecting compounds (2500 μM) at RT for 1 h. The adduct was qualified by mass spectrometry-based assays.

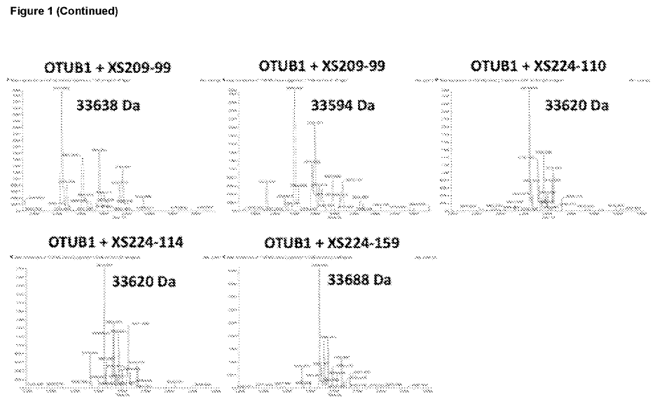

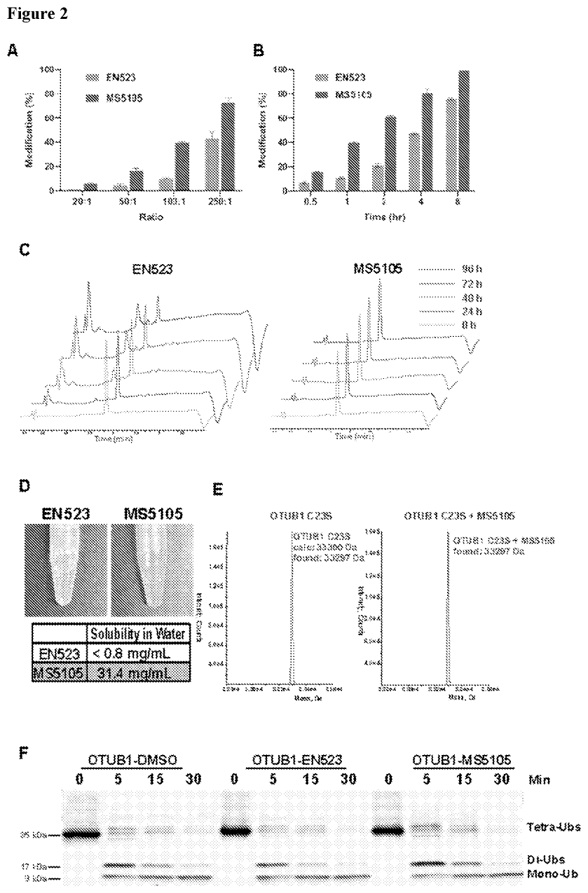

### Example 390. Assessment of the Improved OTUB1 Ligand MS5105 (FIG. 2)

A) Quantification of the OTUB1-ligand adduct formed after EN523 (red) or MS5105 (blue) was incubated with WT OTUB1 at the indicated ligand:OTUB 1 ratio for 1 h. Data shown are the means ±SD from two independent experiments. B) Quantification of the OTUB1-ligand adduct formed after EN523 (red) or MS5105 (blue) was incubated with WT OTUB1 for the indicated time at the 100:1 (ligand:OTUB1) ratio. Data shown are the means±SD from two independent experiments. C) Representative HPLC spectra of EN523 (left) or MS5105 (right) at 1 M concentration after the indicated time in 0.01 M HCl MeOH solution from two independent experiments. D) Representative images of EN523 (0.4 mg) and MS5105 (15.7 mg) in 0.5 mL water and aqueous solubility of EN523 and MS5105. E) Representative mass spectra of the OTUB1 C23S mutant (10 μM) incubated with DMSO (top) or MS5105 (bottom) for 1 h at the 250:1 (ligand:protein) ratio from two independent experiments. F) Representative western blot results of the tetra-ubiquitin (3.6 μM) substrate incubated with OTUB1 (1.5 μM) with or without MS5105 (750 μM) at the indicated time point (0, 0.5, or 1 h) from two independent experiments.

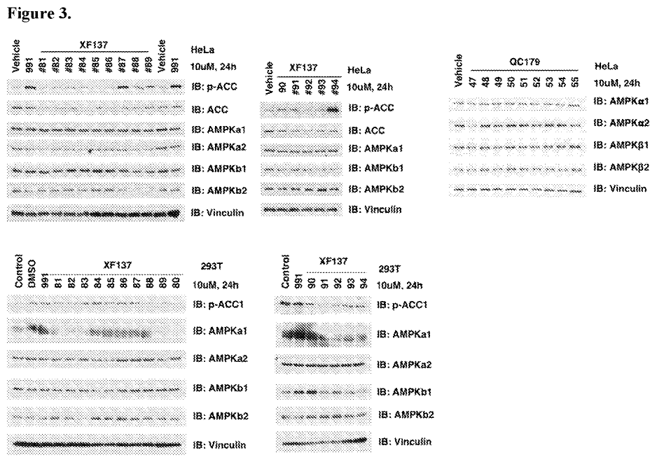

### Example 391. Assessment of the Effect of Selected AMPK Based Bivalent Compounds on Stabilization of the AMPK Protein Level in Hela and 11E.K293T Cells Using WB Assay (FIG. 3)

The Hela or HEK293T cells was treated with DMSO, positive control (991) or selected bivalent compound at 10 μM for 24 h. The Western blot results showed that multiple compounds can increase AMPKβ1 protein level.

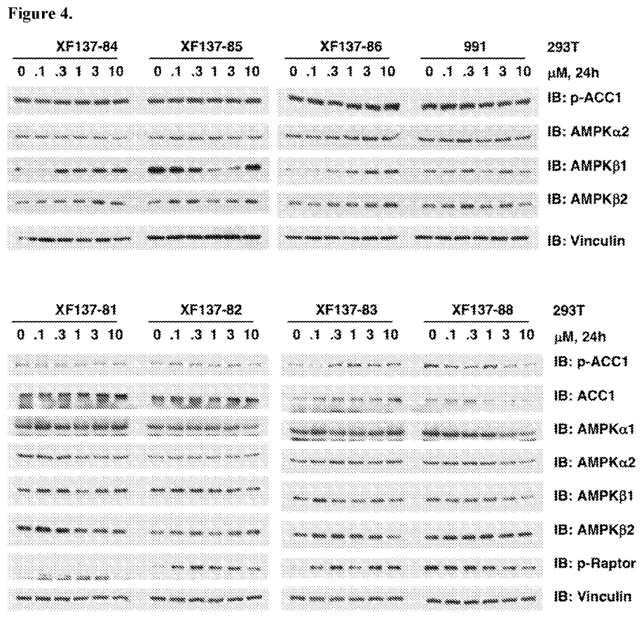

### Example 392. Assessment of Selected AMPK Based Bivalent Compounds in Stabilization of the AMPK Protein Level in 1HEK293T Cells Using WB Assay in Multiple Concentrations (FIG. 4)

The HEK293T cells was treated with DMSO, positive control (991) or selected bivalent compound at indicated concentrations (0.1, 0.3 1, 3, and 10 μM) for 24 h. The Western blot results showed that several compounds dose-dependently increase AMPKβ1 protein level.

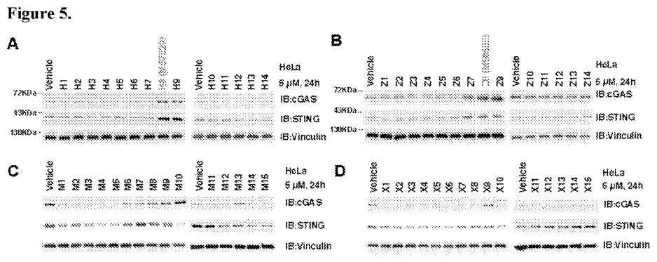

### Example 393. Assessment of the Effect of Selected cGAS Based Bivalent Compounds on Stabilization of the cGAS Protein Level in Hela Cells Using WB Assay (FIG. 5)

The Hela cells was treated with DMSO, or selected bivalent compound at 10 M for 24 h. The Western blot results showed that multiple compounds can increase cGAS and its downstream target STING proteins level.

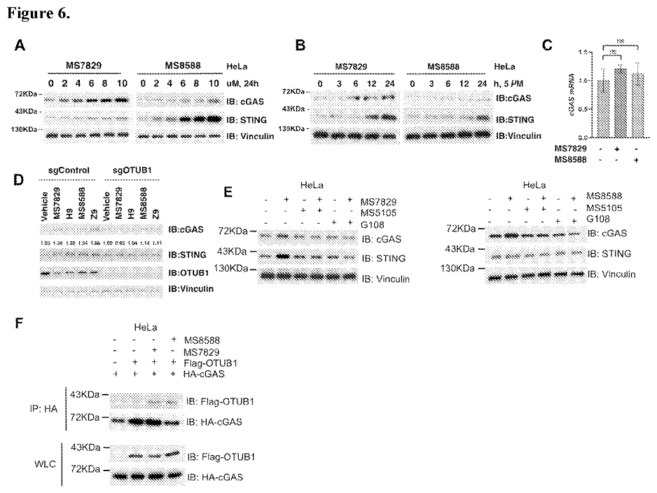

### Example 394. Assessment of the Leading cGAS Based Bivalent Compounds (FIG. 6)

A) WB results of cGAS and STING in HeLa cells treated with MS7829 or MS8588 at the indicated concentration for 24 h. B) WB results of cGAS and STING in HeLa cells treated with 5 μM of MS7829 or MS8588 for the indicated time. C) RT-qPCR analysis of the cGAS mRNA level in HeLa cells treated with DMSO or 5 μM of MS7829 or MS8588 for 24 h. Data shown are the means ±SD from 2 independent experiments. D) WB results of cGAS, STING and OTUB1 in HeLa cells infected with lentivirus of sgOTUB1 to deplete OTUB1 using CRISPR/Cas-9, followed by treatment with the indicated cGAS DUBTAC at 10 μM for 24 h. E) WB result of cGAS protein level in Hela cells treated with (left) MS7829 (5 μM), cGAS antagonist G108 (25 μM), or MS5105 (25 μM), alone or in combination, for 24 h; (right) MS8588 (5 μM), cGAS antagonist G108 (25 μM), or MS5105 (25 M), alone or in combination, for 24 h. F) cGAS co-elutes with OTUB1 in the presence of MS7829 or MS8588.

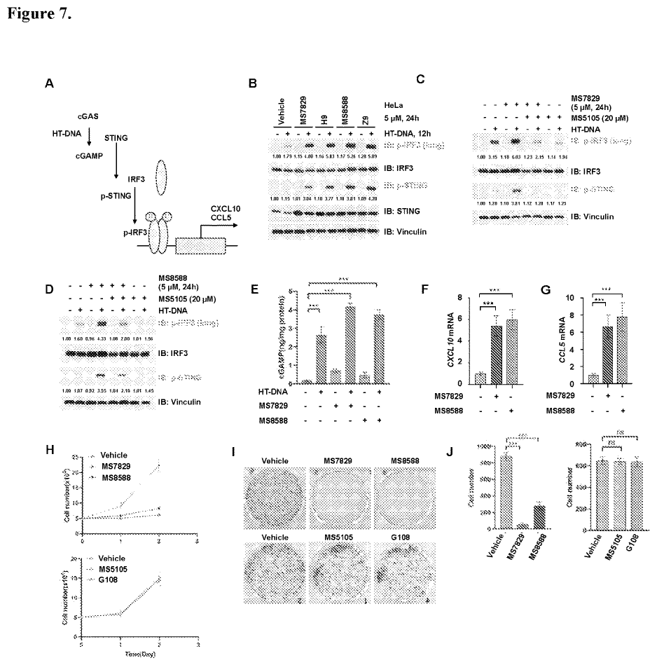

### Example 395. Assessment of Selected cGAS Based Bivalent Compounds in Stabilization of the cGAS Protein Level and Activation of its Downstream Targets in Hela Cells (FIG. 7)

A) A schematic diagram of the cGAS/STING/IRF3 signaling. B) WB results of p-IRF3, IRF3, p-STING and STING in HeLa cells treated with 5 μM of the indicated compound for 24 h followed by stimulation with 1 g/mL HT-DNA for additional 12 h. C-D. WB results of p-IRF3, IRF3 and p-STING in HeLa cells treated with 5 M of MS7829 (C) or MS8588 (D) in the presence or absence of 20 μM of MS5105 for 24 h followed by stimulation with 1 μg/mL HT-DNA for additional 12 h. E) ELISA analysis of the cGAMP level in HeLa cells treated with 5 μM of MS7829 or MS8588 for 24 h followed by stimulation with 1 μg/mL HT-DNA for additional 12 h. Data shown are the means±SD from 2 independent experiments. F-G) RT-qPCR analysis of CXCL10 (F) and CCL5 (G) mRNA levels in HeLa cells treated with 5 μM of MS7829 or MS8588 for 24 h followed by stimulation with 1 μg/mL HT-DNA for additional 12 h. Data shown are the means±SD from 2 independent experiments. H) Growth curves of HeLa cells treated with 5 μM of MS7829, MS8588, (top) MS5105 or G108 (bottom). Data shown are the means±SD from 2 independent experiments. I. Clonogenic assay results of HeLa cells (300/well) treated with 5 M of MS7829, MS8588, MS5105 or G108 for 3 weeks. Cells were fixed and stained with crystal violet. The images shown are representative of two independent experiments. J. Quantification of the clonogenic assay results in panel I. WB results shown in panels B-D are representative of at least 2 independent experiments. For panels E-G and J, ***P<0.001.

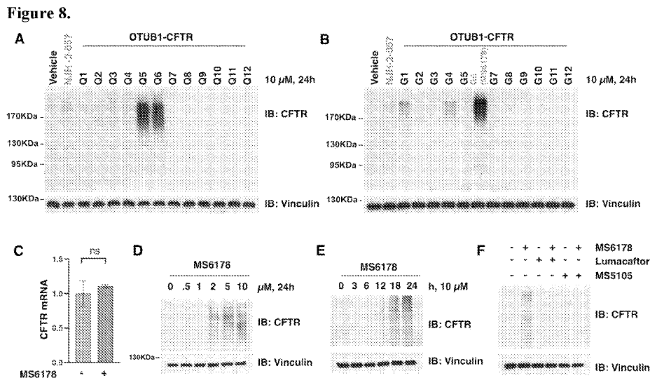

### Example 396. Assessment of the Effect of Selected CFTR Based Bivalent Compounds on Stabilization of the CFTR Protein Level in CFBE41o- 4.7 ΔF508-CFTR Human Cystic Fibrosis Bronchial Epithelial Cells Using WB Assay (FIG. 8)

A-B) Western blot (WB) results of the CFTR protein level in CFBE41o-4.7 ΔF508-CFTR cells treated with 10 μM of the indicated compound for 24 h, with NJH-2-057 as a positive control. C) RT-qPCR analysis of the CFTR mRNA level in CFBE41o-4.7 ΔF508-CFTR cells treated with 10 M of MS6178 for 24 h. Data shown are the means±SD from two independent experiments. D) WB results of the CFTR protein level in CFBE41o-4.7 ΔF508-CFTR cells treated with MS6178 at the indicated concentration for 24 h. E) WB results of the CFTR protein level in CFBE41o-4.7 ΔF508-CFTR cells treated with 10 μM of MS6178 for the indicated time. F) WB results of the CFTR protein level in CFBE41o-4.7 ΔF508-CFTR cells treated with MS6178 (10 μM), Lumacaftor (50 μM), or MS5105 (50 μM), alone or in combination, for 24 h.

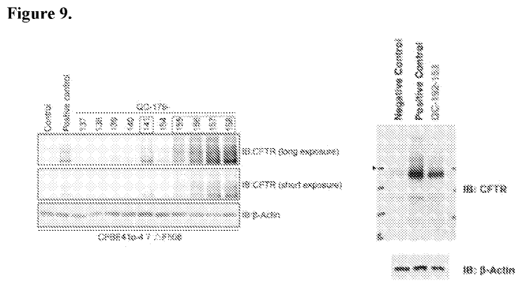

### Example 397. Assessment of the Effect of Selected CFTR Based Bivalent Compounds on Stabilization of the CFTR Protein Level in CFBE41o- 4.7 ΔF508-CFTR Human Cystic Fibrosis Bronchial Epithelial Cells Using WB Assay (FIG. 9)

The CFBE41o- 4.7 ΔF508-CFTR human cystic fibrosis bronchial epithelial cells was treated with DMSO, positive control (NJH-2-057) or selected bivalent compound at 10 μM for 24 h. The Western blot results showed that multiple compounds significantly increase CFTR protein level.

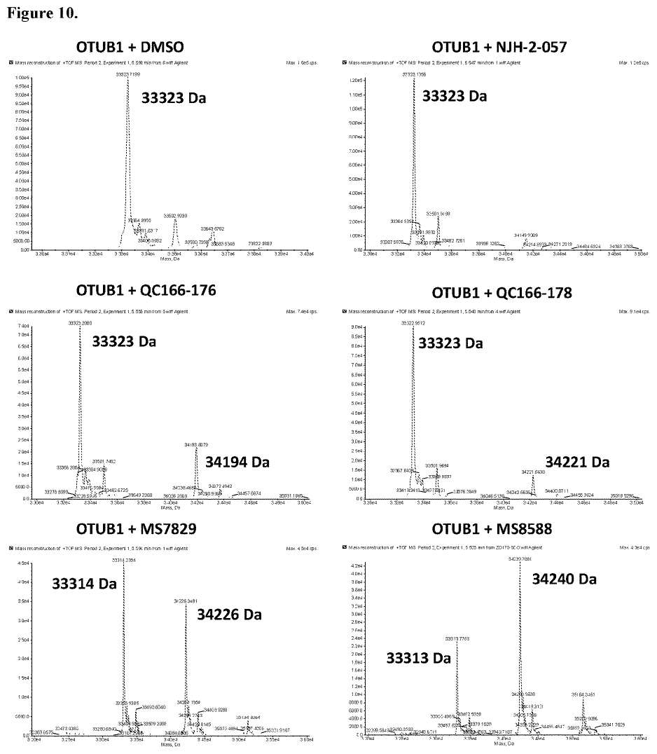

### Example 398. Assessment of the Covalent Modification of OTUB1 Protein by Selected CFTR Based Bivalent Compounds Using Mass Spectrometry-Based Assay (FIG. 10)

OTUB1 protein (10 μM) was incubated with selecting bivalent compounds (2500 μM) at RT for 1 h. The adduct was qualified by mass spectrometry-based assays.

### Materials and Methods:

### General Chemistry Methods:

All chemicals and reagents were purchased from commercial suppliers and used without further purification. HPLC spectra for all compounds were acquired using an Agilent 1200 Series system with DAD detector. Chromatography was performed on a 2.1×150 mm Zorbax 300SB—C18 5 μm column with water containing 0.1% formic acid as solvent A and acetonitrile containing 0.1% formic acid as solvent B at a flow rate of 0.4 ml/min. The linear gradient was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Proton Nuclear Magnetic Resonance (¹H-NMR) spectra were recorded on a Bruker DRX-400 MHz spectrometer, and Carbon Nuclear Magnetic (¹³C NMR) were recorded at 150 MHz. Chemical shifts are expressed in parts per million (ppm) and reported as 6 value (chemical shift 6). Coupling constants are reported in units of hertz (Jvalue, Hz; Integration and splitting patterns: where s=singlet, d=double, t=triplet, q=quartet, brs=broad singlet, m=multiple). Preparative HPLC was performed on Agilent Prep 1200 series with UV detector set to 254 nm. Samples were injected onto a Phenomenex Luna 75×30 mm, 5 μm, C18 column at room temperature. The flow rate was 40 ml/min. A linear gradient was used with 10% (or 50%) of MeOH (A) in H₂O (with 0.1% TFA) (B) to 100% of MeOH (A). HPLC was used to establish the purity of target compounds. All compounds showed >95% purity using the HPLC methods described above.

### Expression and Purification of OTUB1 Proteins

Human OTUB1 wild-type (NM_017670.3) and C23S mutation genes were optimized based on the codon preference of E. coli, synthesized and cloned into pET15b vector with a N-terminal His tag by Genscript, respectively. The plasmid was transferred to BL21-CodonPlus (DE3)-RIPIL Competent Cell (230280, Agilent Technologies) and the recombinant protein was induced expressed by 0.5 mM IPTG at 16° C. with shaking 200 rpm for 18 h. Induced cells were harvested by centrifugation and resuspended in cold lysis buffer (50 mM Tris-H—Cl pH 7.5, 150 mM NaCl, 25 mM imidazole pH 7.5, 0.01% IGEPAL, 5% Glycerol) in the presence of Pierce Protease Inhibitor tablets (PIA32963, Fisher Scientific) and 2 mM TCEP pH 7.5.

The cells were lysed by sonication, clarified by centrifugation, and the filtered supernatant loaded onto a 5 ml HisTrap HP affinity column (17524802, Cytiva) in AKTA system. The His-recombinant protein bound to column was washed using buffer A (50 mM Tris-HCl pH 7.5, 150 mM NaCl, 25 mM imidazole pH 7.5, 0.01% IGEPAL, 5% Glycerol) and eluted with buffer B (50 mM Tris-HCl pH 7.5, 150 mM NaCl, 250 mM imidazole pH 7.5, 0.01% IGEPAL, 5% Glycerol). The eluted fractions containing OTUB1 protein were concentrated and subjected to size exclusion chromatography using a 320 mL HiLoad 26/600 Superdex 200 pg column (28989336, Cytiva) under the buffer condition (50 nM Tris-HCl pH 7.5, 150 mM NaCl, and 2 mM TCEP pH 7.5). The eluted fractions containing OTUB1 protein were concentrated, divided into aliquots and stored at −80° C. freezer for further usage.

The final concentration of OTUB1 wild-type and OTUB1 C23S mutation proteins were 33 mg/mL and 38 mg/mL, respectively. Quality control of the OTUB1 proteins was performed by SDS-PAGE, Nanodrop and mass spectrometry.

### Cell Culture

The CFBE41o- 4.7 ΔF508-CFTR human cystic fibrosis bronchial epithelial cells were purchased from Millipore Sigma (SCC159) CFBE41o- 4.7 ΔF508-CFTR cells are derived from the parental CFBE41o- cells by introduced the ΔF508-CFTR construct. CFBE41o- 4.7 ΔF508-CFTR cells were cultured in α-MEM media (Sigma. #M2279) supplemented with 10% FBS, 2 mM L-Glutamine, 300 μg/mL Hygromycin B, 10,000 units/mL Penicillin and 10,000 μg/mL Streptomycin. For treatment, cells were plated in 6-well plate which is pre-coated with coating mixture (10 pg/mL Fibronectin (Sigma, #F2006), 30 μg/mL PureCol Collagen (Sigma, #5006), and 100 μg/mL BSA(Sigma. #126575)), and treated with indicated drugs when cells reach 60-70% confluency. HeLa cells were cultured in Dulbecco's Modified Eagle's Medium (DMEM) containing 10% fetal bovine serum (FBS), 100 Units of penicillin and 100 μg/ml streptomycin. For treatment, cells in 6-well plate were treated with indicated compounds. To activate the cGAS/STING pathways, cells were transfected with either HT-DNA (Sigma, D6898) with PEl (Polysciences, #23966). For free ligand competition assay, the free ligand, USP7 ligand, cGAS ligand or AMPK ligand in DMSO were co-treated with indicated DUBTAC compounds.

### Western Blot

Cells were lysed in EBC buffer (120 mM NaCl, 50 mM Tris-Cl pH 8.0, 0.5% NP-40) supplemented with protease inhibitors cocktail (Pierce) and phosphatase inhibitors (phosphatase inhibitor cocktail set I and II, Calbiochem). The protein samples were resolved by 7% SDS-PAGE at 130 V for 80 min, and immunoblotted with indicated antibodies at 4° C. overnight, washed four times with Tris-buffered saline with 0.1% Tween-20 (TBST) buffer, incubated with secondary antibody in 5% non-fat milk for 1 hour at room temperature, and then washed another four times with TBST buffer. CFTR (#78335, 1:1,000) antibody was purchased from Cell Signaling Technologies. Anti-Actin (A2228, 1:50,000), anti-vinculin antibody (V-4505, 1:50,000), peroxidase-conjugated anti-mouse secondary antibody (A-4416, 1:3000) and peroxidase-conjugated anti-rabbit secondary antibody (A-4914, 1:3,000) were purchased from Sigma. All primary antibodies were diluted in 5% bovine serum albumin (BSA) in TBST buffer, and secondary antibodies were diluted in 5% non-fat milk in TBST buffer.

### Mass Spectrometry-Based Analysis of OTUB1-Covalent Ligand Adducts

To screen the covalent ligands, recombinant OTUB1 protein (10 μM) was respectively incubated with DMSO, EN523 or seven novel ligands (2500 μM) in reaction buffer (50 mM Tris-HCl pH 7.5, 150 mM NaCl, and 0.5 mM TCEP pH 7.5) for 1 h at room temperature.

For quantitative analysis of the effect of different protein:ligand ratios, recombinant OTUB1 protein (10 μM) was respectively incubated with EN523 or MS5105 (200 μM, 500 μM, 1000 μM, 2500 μM) in reaction buffer for 1 h at room temperature.

To quantitatively assess the time course of the OTUB1 covalent modification by the ligands, recombinant OTUB1 protein (10 μM) was respectively incubated with EN523 or MS5105 (1000 μM) in reaction buffer for 0.5 h, 1 h, 2 h, 4 h, 8 h at room temperature.

Then, the total 500 μL reaction solution was exchanged and concentrated to 50 μL 50 mM ammonium bicarbonate with a 10 kDa MWCO centrifugal filter unit (UFC901024, Millipore) and diluted in MS buffer (30% ACN, 0.2% FA) to a final concentration of 10 M. Mass spectrometry detection was performed by an Agilent LC/MSD Time-Of-Flight (TOF) mass spectrometer (Agilent Technologies) equipped with an electrospray ion source. The data was analyzed by TOF Protein Confirmation Software. All experiments were performed in duplicate.

### In Vitro Deubiquitinase Assay

Recombinant OTUB1 protein (10 μM) was preincubated with DMSO or MS5105 (5000 μM) in reaction buffer for 1 h at room temperature. Then, the pretreated protein was purified with a 10 kDa MWCO centrifugal filter unit (UFC901024, Millipore).

To initiate the assay, untreated or pretreated OTUB1 (1.5 μM) was incubated with K48-Linked Tetra-Ubiquitin (3.7 μM, SI-4804-0025, LifeSensors) in reaction buffer for 0, 5, 15, and 30 min at 37° C. The reaction was terminated by adding 4x Laemmli Sample Buffer (1610747, BIO-RAD) with 100 mM TECP PH 7.5 and heating at 95° C. for 6 min.

The appearance of tetra-ubiquitin and mono-ibiquitin was monitored with western blotting. The primary antibody was Ubiquitin (P4D1) Mouse mAb (3936S, Cell Signaling Technology). The secondary antibody was IRDye 680RD Donkey anti-Mouse IgG (926-68072, LI-COR). Protein signals were detected by OdysseyCLx imaging system (L1-COR) and then analyzed by image Studio Lite software (LI-COR).

Measurement of cGAMP Level

The cGAMP level was measured using 2′3′-cGAMP ELISA kit (Cayman, #501700) following the manufacturer's manual. Briefly, cells were lysed using the M-PERtm Mammalian Protein Extraction Reagent (ThermoFisher, #78503), and 100 μL cell lysates were used for analysis. The OD450 were measured for calculation of cGAMP level, and finally normalized with protein concentration.

### RT-qPCR

Total RNAs were extracted using Qiagen RNeasy mini kit (Qiagen. #74106) and reversed transcripted into cDNA using iScript™ Reverse Transcription Supermix (Bio-Rad, #1708841). RT-qPCR was performed with SYBR Select Master Mix (ThermoFisher, #4472908) using indicated primers.

### Clonal Formation Assay

HeLa cells in a 6-well plate were treated with the indicated DUBTAC for 24 hours. Two weeks later, cells were fixed in fixation buffer (acetic acid: methanol=1:7) and stained with 0.4% crystal violet in 20% ethanol. The clonal numbers were quantified using ImageJ software.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Aledavood, E., Gheeraert, A., Forte, A., Vuillon, L., Rivalta, I., Luque, F. J., and Estarellas, C. (2021). Elucidating the activation mechanism of AMPK by direct pan-activator PF-739. Front. Mol. Biosci. 8, 760026. 10.3389/fmolb.2021.760026.
Cusi, K., Alkhouri, N., Harrison, S. A., Fouqueray, P., Moller, D. E., Hallakou-Bozec, S., Bolze, S., Grouin, J. M., Jeannin Megnien, S., Dubourg, J., and Ratziu, V. (2021). Efficacy and safety of PXL770, a direct AMP kinase activator, for the treatment of non-alcoholic fatty liver disease (STAMP-NAFLD): a randomised, double-blind, placebo-controlled, phase 2a study. Lancet Gastroenterol Hepatol. 6, 889-902. 10.1016/s2468-1253(21)00300-9.
Dale, B., Anderson, C., Park, K.-S., Kaniskan, H. U., Ma, A., Shen, Y., Zhang, C., Xie, L., Chen, X., Yu, X., and Jin, J. (2022). Targeting Triple-Negative Breast Cancer by a Novel Proteolysis Targeting Chimera Degrader of Enhancer of Zeste Homolog 2. ACS Pharmacol. Transl. Sci. 5, 491-507. 10.1021/acs ptsci.2c00100.
Edmonds, D. J., Kung, D. W., Kalgutkar, A. S., Filipski, K. J., Ebner, D. C., Cabral, S., Smith, A. C., Aspnes, G. E., Bhattacharya, S. K., Borzilleri, K. A., et al. (2018). Optimization of metabolic and renal clearance in a series of indole acid direct activators of 5′-Adenosine Monophosphate-Activated Protein Kinase (AMPK). J. Med. Chem. 61, 2372-2383. 10.1021/acs.jmedchem.7b01641.
Figarola, J. L., and Rahbar, S. (2013). Small-molecule COH—SR4 inhibits adipocyte differentiation via AMPK activation. Int. J. Mol. Med. 31, 1166-1176. 10.3892/ijmm.2013.1313.
Henning, N. J., Boike, L., Spradlin, J. N., Ward, C. C., Liu, G., Zhang, E., Belcher, B. P., Brittain, S. M., Hesse, M. J., Dovala, D., et al. (2022). Deubiquitinase-targeting chimeras for targeted protein stabilization. Nat. Chem. Biol. 18, 412-421. 10.1038/s41589-022-00971-2.
Jeon, S. M. (2016). Regulation and function of AMPK in physiology and diseases. Exp. Mol. Med. 48, e245. 10.1038/emm.2016.81.
Kim, Y. M., Kim, M. Y., Kim, H. J., Roh, G. S., Ko, G. H., Seo, H. G., Lee, J. H., and Chang, K. C. (2011). Compound C independent ofAMPK inhibits ICAM-1 and VCAM-1 expression in inflammatory stimulants-activated endothelial cells in vitro and in vivo. Atherosclerosis 219, 57-64. 10.1016/j.atherosclerosis.2011.06.043.
Lama, L., Adura, C., Xie, W., Tomita, D., Kamei, T., Kuryavyi, V., Gogakos, T., Steinberg, J. I., Miller, M., Ramos-Espiritu, L., et al. (2019). Development of human cGAS-specific small-molecule inhibitors for repression of dsDNA-triggered interferon expression. Nat. Comm. 10, 2261. 10.1038/s41467-019-08620-4.
Lemos, C., Schulze, V. K., Baumgart, S. J., Nevedomskaya, E., Heinrich, T., Lefranc, J., Bader, B., Christ, C. D., Briem, H., Kuhnke, L. P., et al. (2021). The potent AMPK inhibitor BAY-3827 shows strong efficacy in androgen-dependent prostate cancer models. Cell Oncol (Dordr) 44, 581-594. 10.1007/s13402-020-00584-8.
Liu, J., Yu, X., Chen, H., Kaniskan, H. Ü., Xie, L., Chen, X., Jin, J., and Wei, W. (2022). TF-DUBTACs stabilize tumor suppressor transcription factors. J. Am. Chem. Soc 144, 12934-12941. 10.1021/jacs.2c04824.
Myers, R. W., Guan, H. P., Ehrhart, J., Petrov, A., Prahalada, S., Tozzo, E., Yang, X., Kurtz, M. M., Trujillo, M., Gonzalez Trotter, D., et al. (2017). Systemic pan-AMPK activator MK-8722 improves glucose homeostasis but induces cardiac hypertrophy. Science 357, 507-511. 10.1126/science.aah5582.
Nakamaru, K., Matsumoto, K., Taguchi, T., Suefuji, M., Murata, Y., Igata, M., Kawashima, J., Kondo, T., Motoshima, H., Tsuruzoe, K., et al. (2005). AICAR, an activator of AMP-activated protein kinase, down-regulates the insulin receptor expression in HepG2 cells. Biochem.
Biophys. Res. Commun. 328, 449-454. 10.1016/j.bbrc.2005.01.004. Ngoei, K. R. W., Langendorf, C. G., Ling, N. X. Y., Hoque, A., Varghese, S., Camerino, M. A., Walker,
S. R., Bozikis, Y. E., Dite, T. A., Ovens, A. J., et al. (2018). Structural determinants for small-molecule activation of skeletal muscle AMPK α2β2γ1 by the glucose importagog SC4. Cell Chem. Biol. 25, 728-737.e729. 10.1016/j.chembiol.2018.03.008.
Rhodes, N., Heerding, D. A., Duckett, D. R., Eberwein, D. J., Knick, V. B., Lansing, T. J., McConnell, R. T., Gilmer, T. M., Zhang, S. Y., Robell, K., et al. (2008). Characterization of an Akt kinase inhibitor with potent pharmacodynamic and antitumor activity. Cancer Res. 68, 2366-2374. 10.1158/0008-5472.Can-07-5783.
Samson, N., and Ablasser, A. (2022). The cGAS-STI NG pathway and cancer. Nat. Cancer 3, 1452-1463. 10.1038/s43018-022-00468-w.
Sanders, M. J., Ali, Z. S., Hegarty, B. D., Heath, R., Snowden, M. A., and Carling, D. (2007). Defining the mechanism of activation of AMP-activated protein kinase by the small molecule A-769662, a member of the thienopyridone family. J. Biol. Chem. 282, 32539-32548. 10.1074/jbc.M706543200.
Sun, G., You, Y., Li, H., Cheng, Y., Qian, M., Zhou, X., Yuan, H., Xu, Q. L., Dai, L., Wang, P., et al. (2020). Discovery of AdipoRon analogues as novel AMPK activators without inhibiting mitochondrial complex I. Eur. J. Med. Chem. 200, 112466. 10.1016/j.ejmech.2020.112466.
Tan, L., Shan, H., Han, C., Zhang, Z., Shen, J., Zhang, X., Xiang, H., Lu, K., Qi, C., Li, Y., et al. (2022). Discovery of potent OTUB1/USP8 dual inhibitors targeting proteostasis in non-small-cell lung cancer. J. Med. Chem. 65, 13645-13659. 10.1021/acs.jmedchem.2c00408.
Vingtdeux, V., Chandakkar, P., Zhao, H., Davies, P., and Marambaud, P. (2011). Small-molecule activators of AMP-activated protein kinase (AMPK), RSVA314 and RSVA405, inhibit adipogenesis. Mol. Med. 17, 1022-1030. 10.2119/molmed.2011.00163.
Ward, C. L., Omura, S., and Kopito, R. R. (1995). Degradation of CFTR by the ubiquitin-proteasome pathway. Cell 83, 121-127. 10.1016/0092-8674(95)₉₀₂₄₀-6.
Xiao, D., Lu, Z., Wang, Z., Zhou, S., Cao, M., Deng, J., Hu, X., Peng, M., He, C., Wu, J., et al. (2020). Synthesis, biological evaluation and anti-proliferative mechanism of fluorine-containing proguanil derivatives. Bioorg. Med. Chem. 28, 115258. 10.1016/j.bmc.2019.115258.
Zhao, J., Xiao, R., Zeng, R., He, E., and Zhang, A. (2022). Small molecules targeting cGAS-STING pathway for autoimmune disease. E. J. Med. Chem. 238, 114480. https://doi.org/10.1016/j.ejmech.2022.114480.

What is claimed is:

1. An OTUB1 binder comprising a compound according to Formula (A-I): 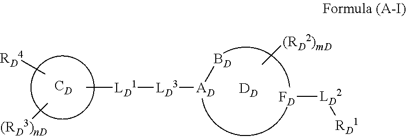
wherein
A_{D} is selected from N or CR_{D}²;
B_{D} is selected from C═O, CH₂, CH R_{D}², or C(R_{D}²)₂;
Ring C_{D} is absent, or selected from C₃-C₁₂ cycloalkyl, 3-12-membered heterocyclic, C₆-C₁₀ aryl, and 5-10 membered heteroaryl;
Ring D_{D} is a saturated or partially unsaturated 4-12 membered heterocyclic;
F_{D} is selected from N, or CR_{D}²;
L_{D}¹ is a bond, or a bivalent group selected from —O—, —NR_{D}⁵—, —C(O)—, —C(O)O—, —C(O)NR_{D}⁵-—NR_{D}⁵C(O)—, —OC(O)NR_{D}⁵—, —NR_{D}⁵C(O)O—, —NR_{D}⁶C(O)NR_{D}⁵—, —S(O)—, —S(O)₂—, —S(O)NR_{D}⁵—, —S(O)₂NR_{D}⁵—, C₁-C₆ alkylene, C₁-C₆ haloalkylene, C₁-C₆ heteroalkylene, C₂-C₆ alkenylene, and C₂-C₆ alkynylene;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₆ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸,—NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring C_{D}, optionally form fused rings or bridged rings;
R_{D}⁴ is R_{D}^{4a} or R_{D}^{4b};
R_{D}^{4a} is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}^{4b} is optionally a bivalent group for connection to a linker, and is selected from a bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene,
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
m_{D} is an integer of 0-8;
n_{D} is an integer of 0-8; and tautomers and pharmaceutically acceptable salts thereof.

2. An OTUB1 binder comprising a compound according to one of Formulae (A-I-a), (A-I-b), (A-I-c) and (A-I-d): 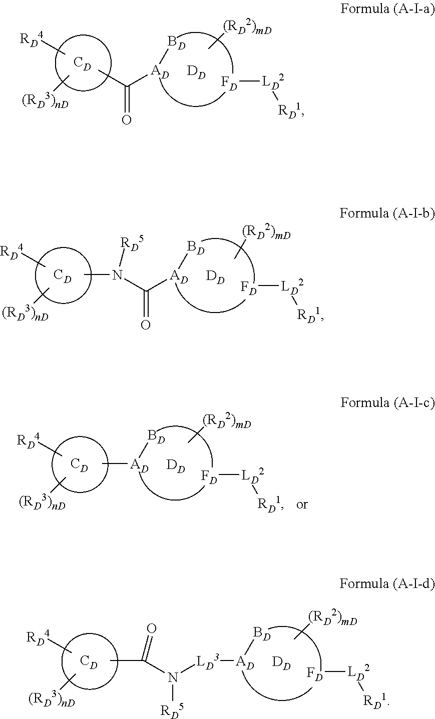
wherein
A_{D} is selected from N or CR_{D}²;
B_{D} is selected from C═O, CH₂, CH R_{D}², or C(R_{D}²)₂;
Ring C_{D} is absent, or selected from C₃-C₁₂ cycloalkyl, 3-12-membered heterocyclic, C₆-C₁₀ aryl, and 5-10 membered heteroaryl;
Ring D_{D} is a saturated or partially unsaturated 4-12 membered heterocyclic;
F_{D} is selected from N, or CR_{D}²;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₆ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸,—NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring C_{D}, optionally form fused rings or bridged rings;
R_{D}⁴ is R_{D}^{4a} or R_{D}^{4b};
R_{D}^{4a} is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}^{4b} is optionally a bivalent group for connection to a linker, and is selected froma bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene;
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
m_{D} is an integer of 0-8;
n_{D} is an integer of 0-8; and
tautomers and pharmaceutically acceptable salts thereof.

3. An OTUB1 binder comprising a compound according to one of Formulae (A-I-a1), (A-I-b1), (A-I-c1) and (A-I-d1): 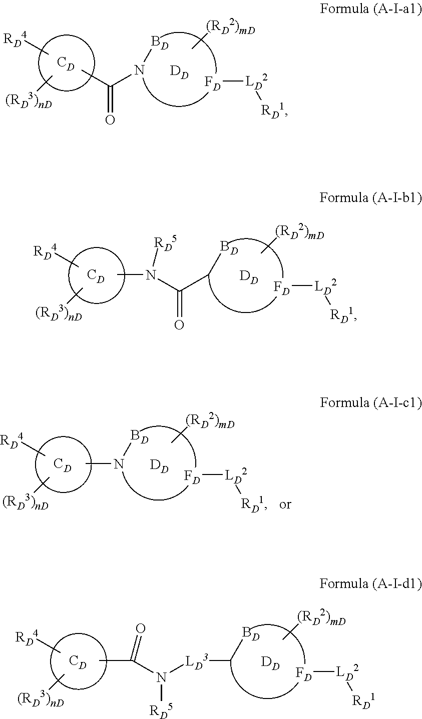
wherein
B_{D} is selected from C═O, CH₂, CH R_{D}², or C(R_{D}²)₂;
Ring C_{D} is absent, or selected from C₃-C₁₂ cycloalkyl, 3-12-membered heterocyclic, C₆-C₁₀ aryl, and 5-10 membered heteroaryl;
Ring D_{D} is a saturated or partially unsaturated 4-12 membered heterocyclic;
F_{D} is selected from N, or CR_{D}²;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₆ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸,—NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring C_{D}, optionally form fused rings or bridged rings;
R_{D}⁴ is R_{D}^{4a} or R_{D}^{4b};
R_{D}⁴a is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}^{4b} is optionally a bivalent group for connection to a linker, and is selected from a bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene;
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
m_{D} is an integer of 0-8;
n_{D} is an integer of 0-8; and
tautomers and pharmaceutically acceptable salts thereof.

4. An OTUB1 binder comprising a compound according to one of Formulae (A-I-a2), (A-I-a3), (A-I-a4), (A-I-a5), (A-I-b2), (A-I-b3), (A-I-b4), (A-I-b5), (A-I-c2), (A-I-c3), (A-I-c4), (A-I-c5), (A-I-d2), (A-I-d3), (A-I-b4) and (A-I-d5): 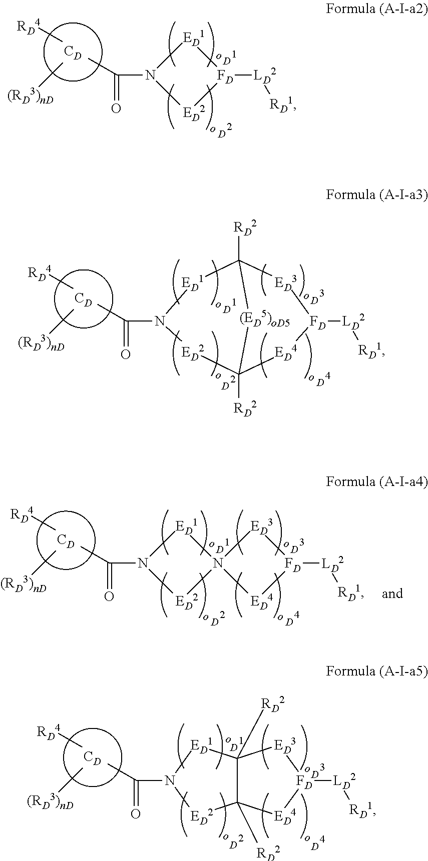 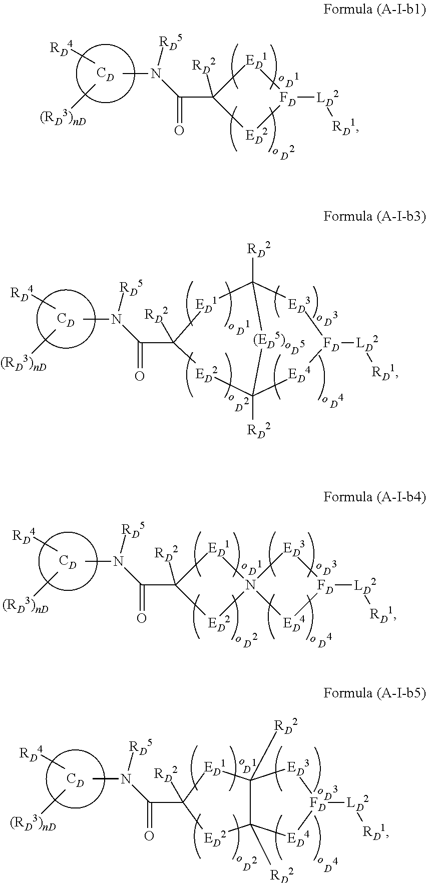 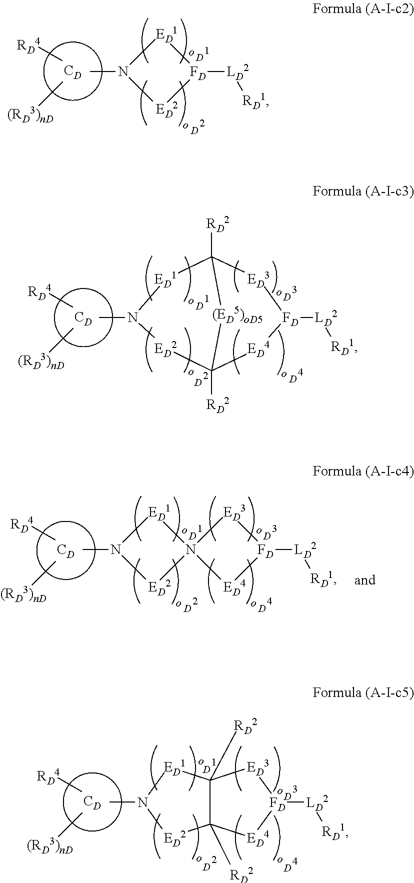 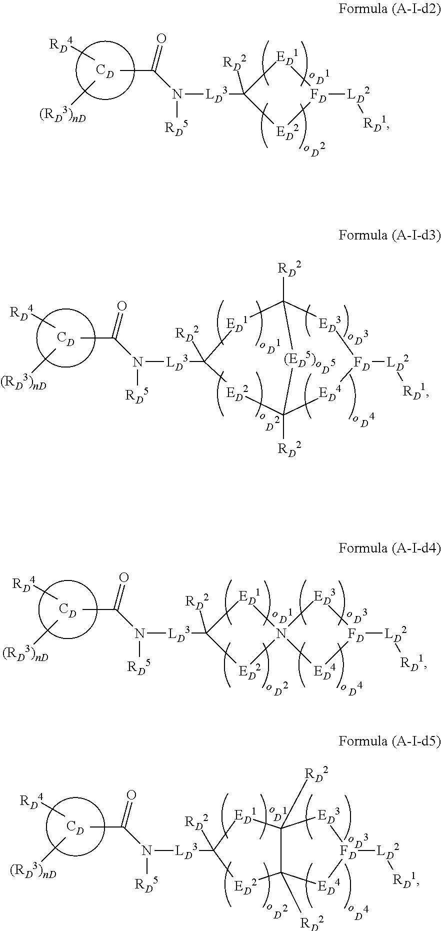
wherein
Ring C_{D} is absent, or selected from C₃-C₁₂ cycloalkyl, 3-12-membered heterocyclic, C₆-C₁₀ aryl, and 5-10 membered heteroaryl;
F_{D} is selected from N, or CR_{D}²;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₆ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is independently selected from hydrogen, halogen, cyano, OR_{D}⁷, NR_{D}⁷R_{D}⁸, C₁-C₆alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸,—NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring C_{D}, optionally form fused rings or bridged rings;
R_{D}⁴ is R_{D}^{4a} or R_{D}^{4b};
R_{D}⁴a is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}^{4b} is optionally a bivalent group for connection to a linker, and is selected from a bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene;
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
n_{D} is an integer of 0-8; and
each E_{D}¹, each E_{D}², each E_{D}³, each E_{D}⁴, and each E_{D}⁵ are independently selected from null, O, CO, SO, S(O)₂, NR_{D}², and CR_{D}²R_{D}², with the proviso that no two oxygen atoms are connected to each other;
o_{D}¹ is selected from an integer of 1-4;
o_{D}², o_{D}³, o_{D}⁴, and o_{D}⁵ are independent selected from an integer of 0-4; and tautomers and pharmaceutically acceptable salts thereof.

5. An OTUB1 binder comprising a compound according to one of Formulae (A-I-a6), (A-I-a7), (A-I-a8), (A-I-a9), (A-I-b6), (A-I-b7), (A-I-b8), (A-I-b9), (A-I-c6), (A-I-c7), (A-I-c8), (A-I-c9), (A-I-d6), (A-I-d7), (A-I-d8), and (A-I-d9): 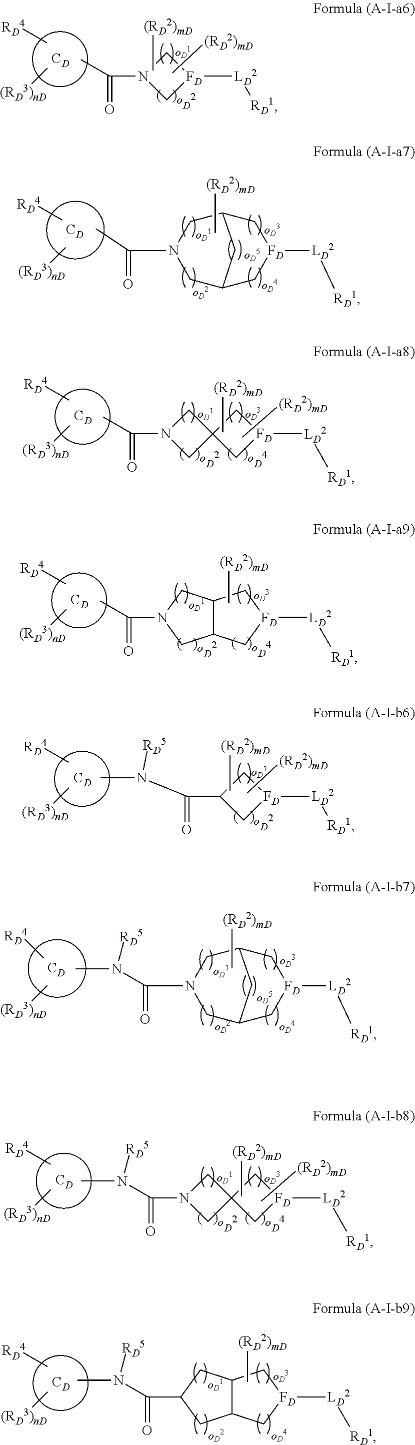 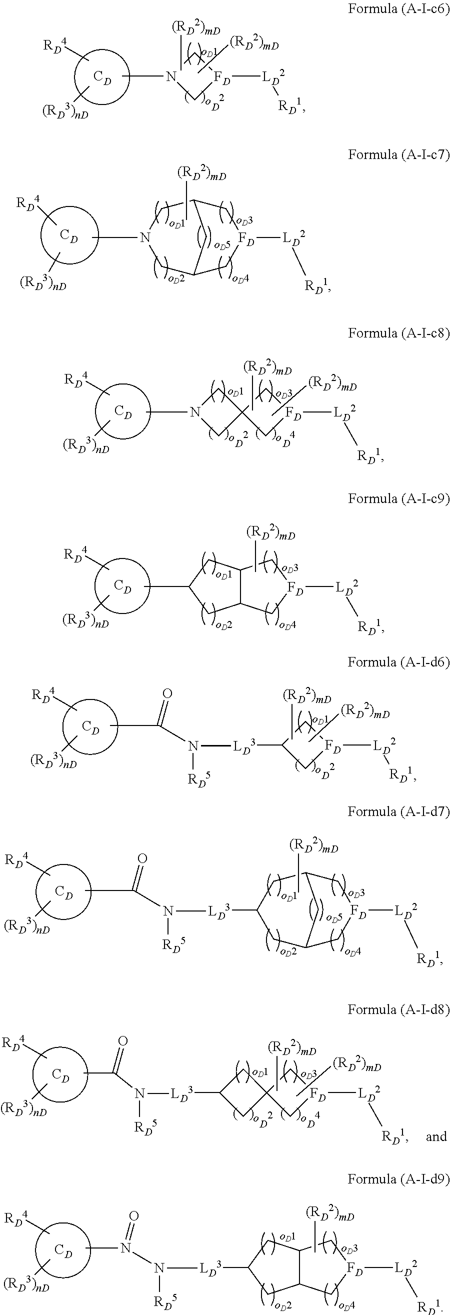
wherein
ring C_{D} is absent, or selected from C₃-C₁₂ cycloalkyl, 3-12-membered heterocyclic, C₆-C₁₀ aryl, and 5-10 membered heteroaryl;
F_{D} is selected from N, or CR_{D}²;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₀ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is a substituent that can attach to anywhere on the monocyclic and bicyclic rings and is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸,—NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring C_{D}, optionally form fused rings or bridged rings;
R_{D}⁴ is R_{D}^{4a} or R_{D}^{4b};
R_{D}⁴a is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}⁴b is optionally a bivalent group for connection to a linker, and is selected from a bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene;
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
o_{D}¹ is selected from an integer of 1-4;
o_{D}², o_{D}³, o_{D}⁴, and o_{D}⁵ are independent selected from an integer of 0-4;
m_{D} is an integer of 0-8;
n_{D} is an integer of 0-8, and
tautomers and pharmaceutically acceptable salts thereof.

6. An OTUB1 binder according to one of claims 1-3, wherein Ring D_{D} is selected from

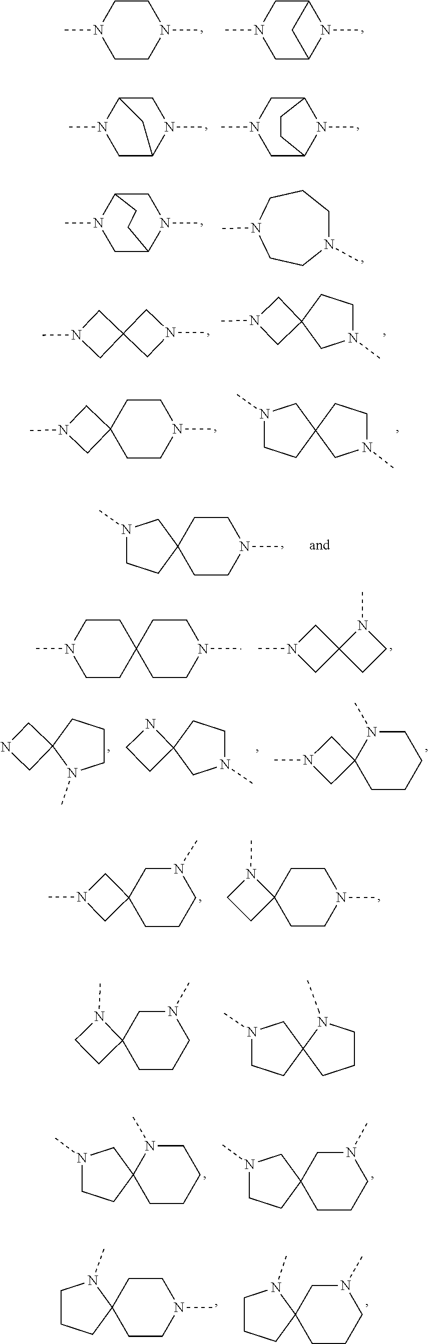

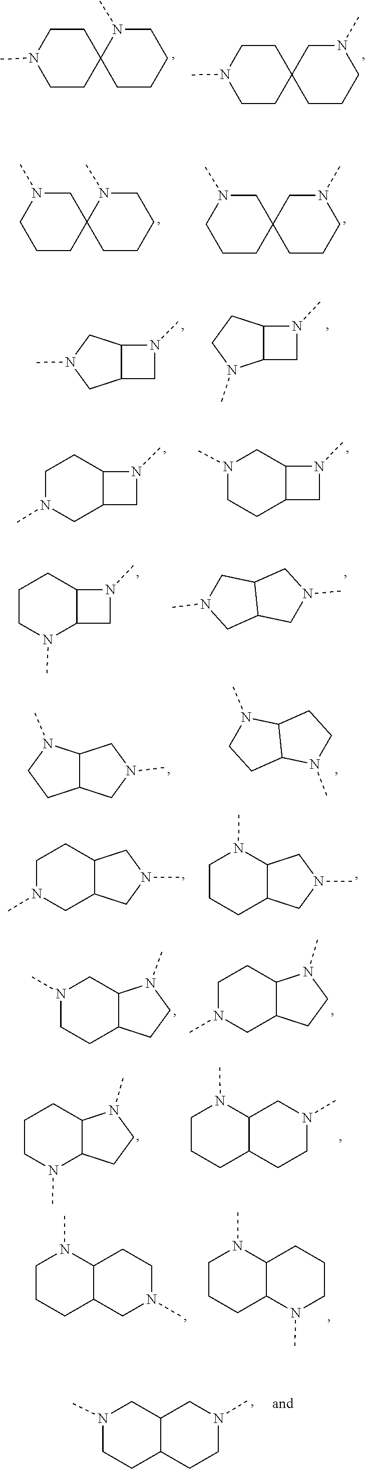

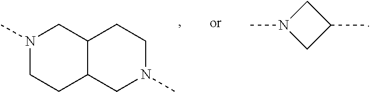

7. An OTUB1 binder according to any one of claims 1-3, wherein
Ring D_{D} is selected from:

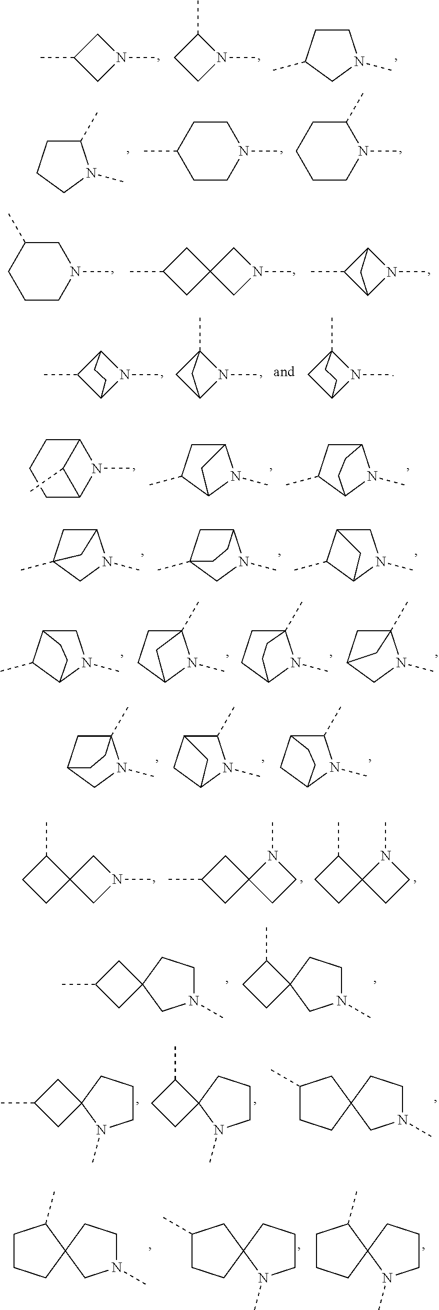

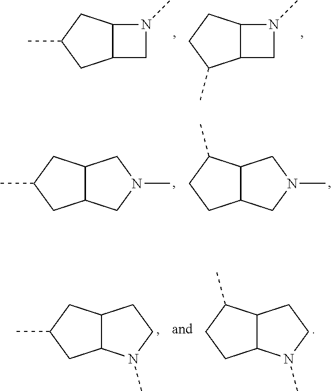

8. An OTUB1 binder according to any one of claim 1-3, wherein
Ring D_{D} is selected from:

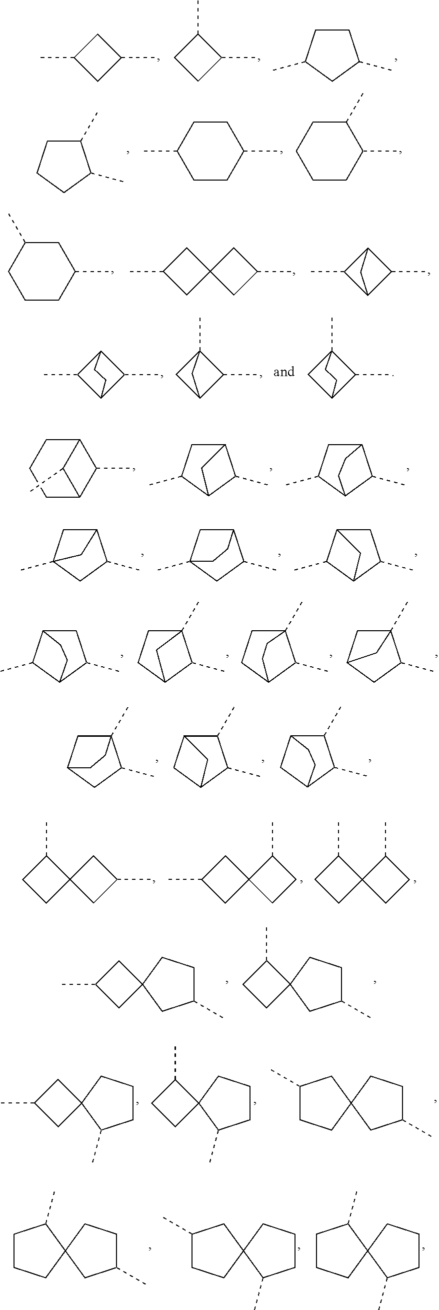

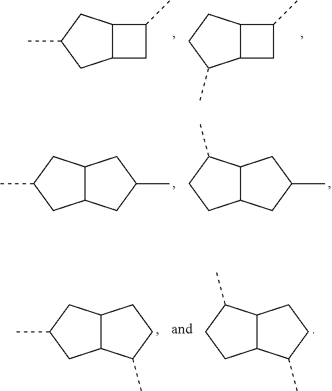

9. An OTUB1 binder according to any one of claims 1-3, wherein
Ring D_{D} is selected from:

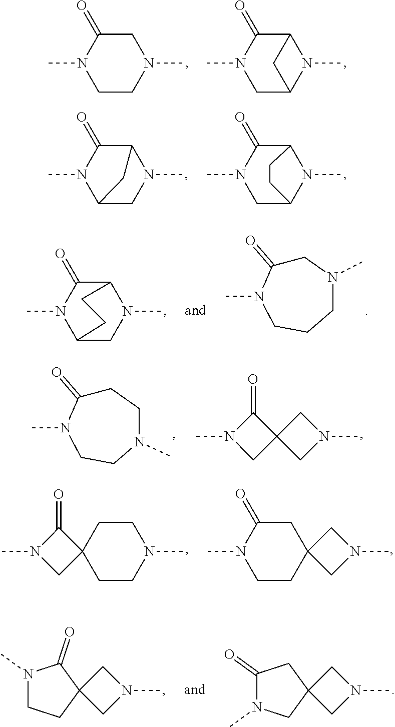

10. An OTUB1 binder according to any one of claim 1-5, wherein ring C_{D} is selected from azetidinyl, pyrrolidinyl, piperidinyl, and piperazinyl thiophenyl, benzothiophenyl, tetrahydrobenzothiophenyl, thiazolyl, imidazolyl, furanyl, pyrrolyl, pyrazolyl, pyridinyl, pyrimidinyl, pyridazinyl, pyrazinyl, benzofuranyl, indolyl, and indazolyl.

11. An OTUB1 binder according to any one of claims 1-10 wherein -L_{D}²-R_{D}¹ is selected from

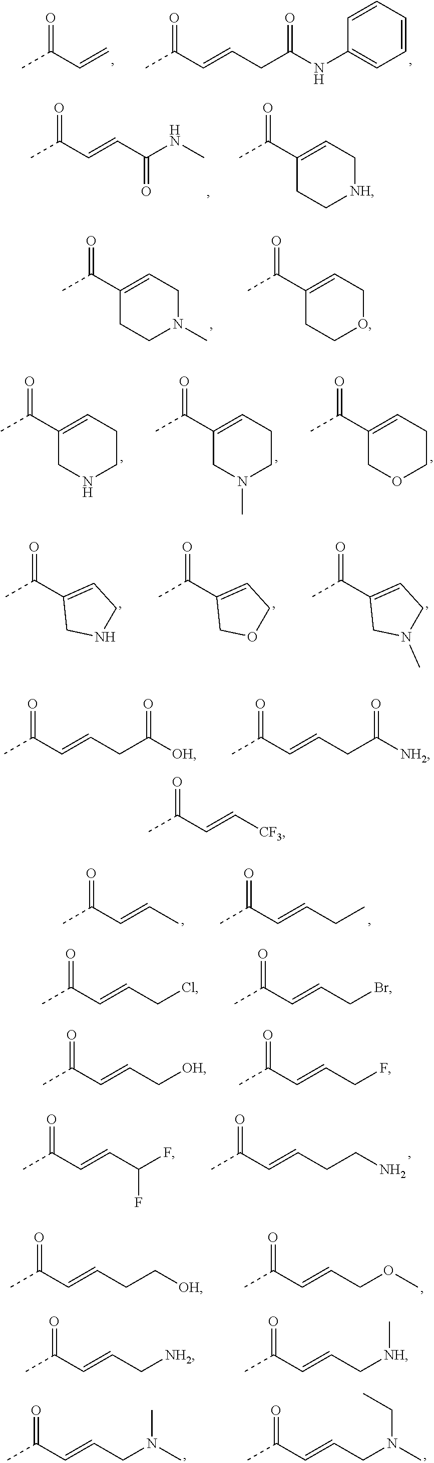

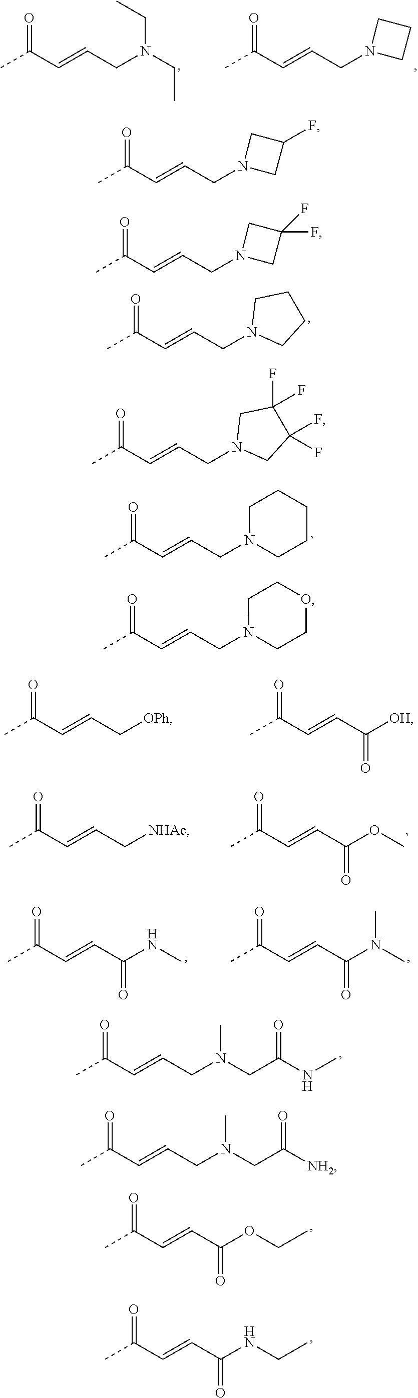

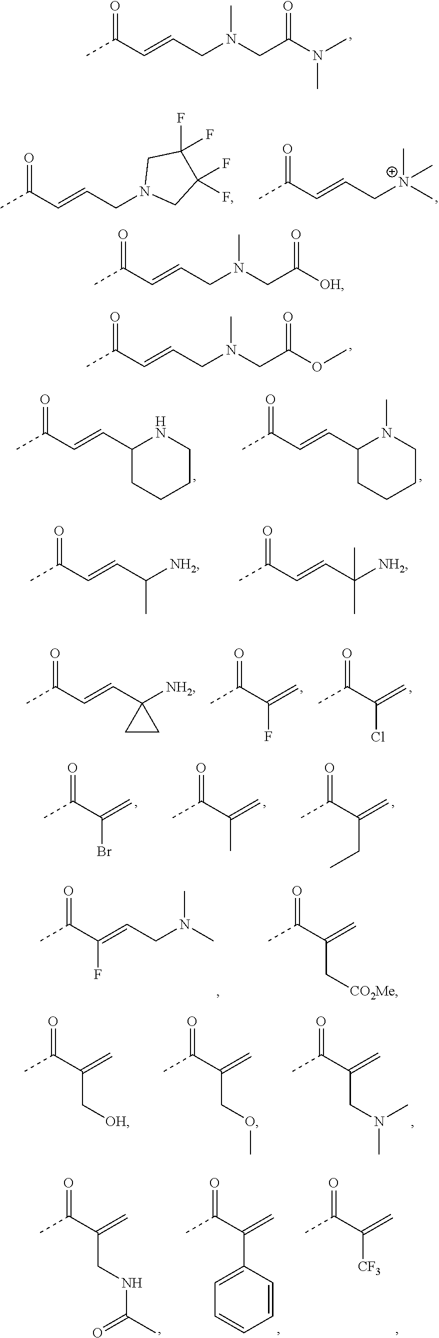

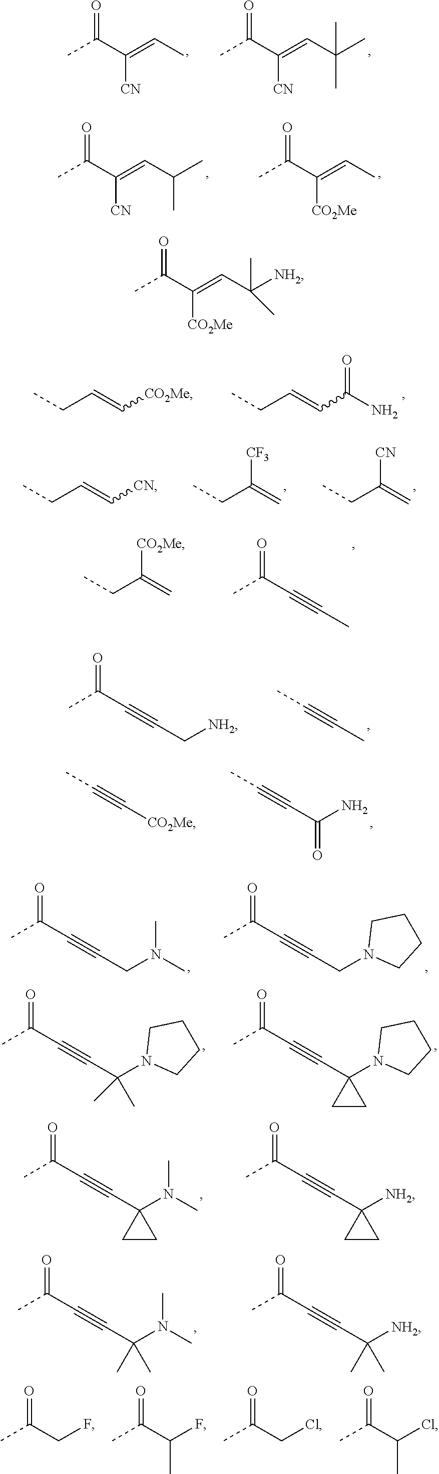

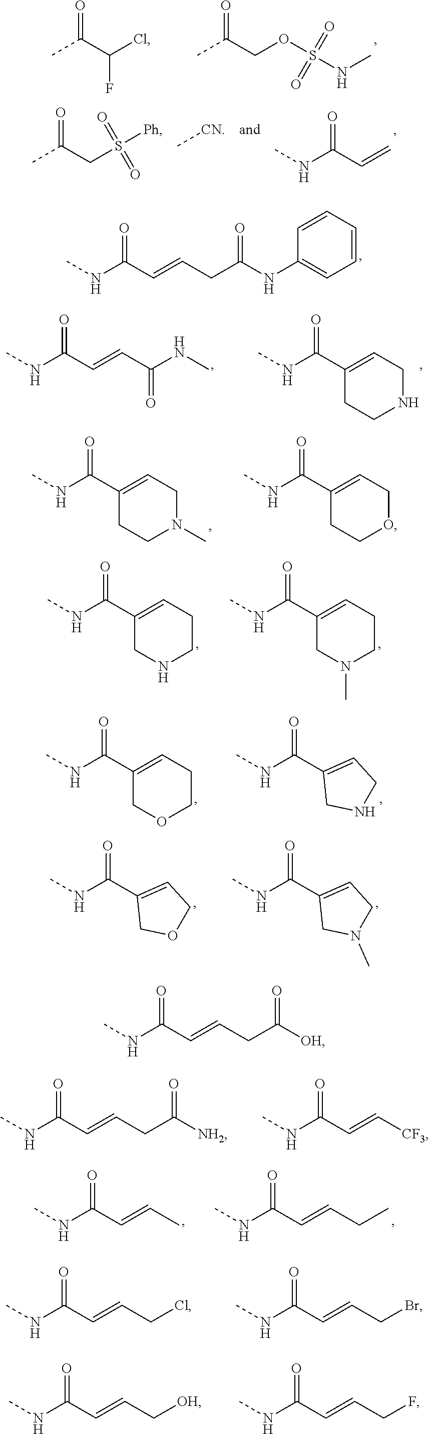

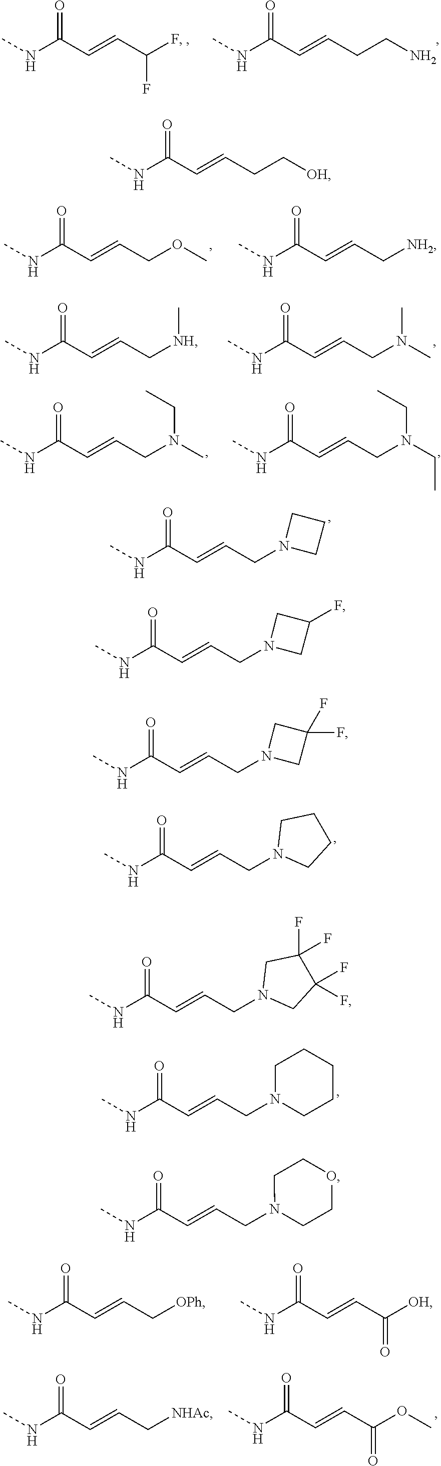

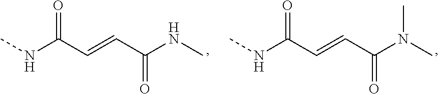

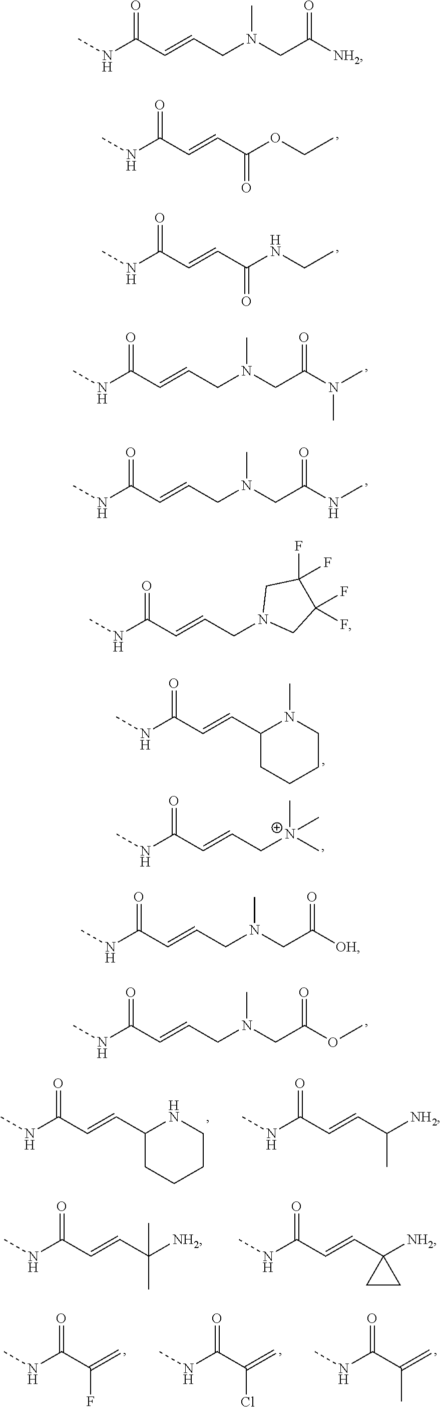

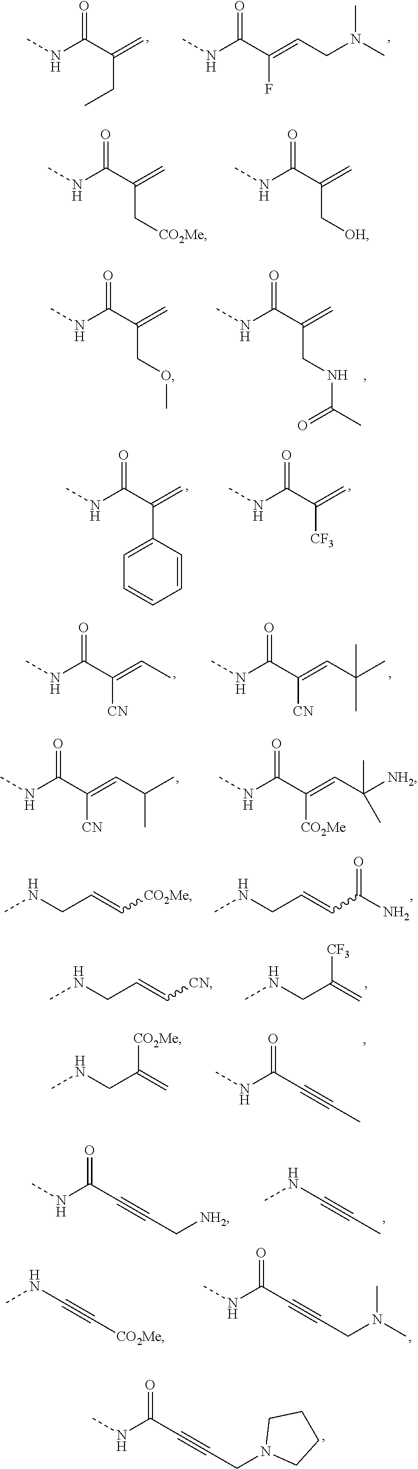

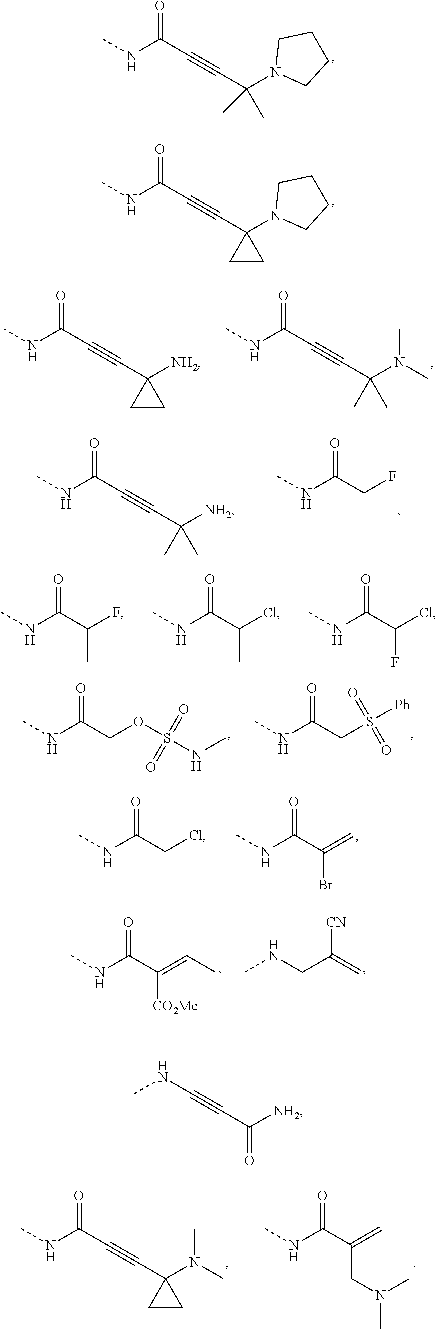

12. An OTUB1 binder selected from XS154-91, XS154-130, XS154-148, XS154-114, XS154-149, XS159-13, XS159-107, XS165-30, XS159-90, XS165-53, XS165-38, XS165-33, XS165-54, XS154-184, XS165-75, XS165-77, XS165-127, XS165-106, XS165-112, XS165-97, XS165-118, XS165-110, XS165-119, XS165-123, XS165-126, XS165-120, XS165-121, XS165-100, XS165-99, XS165-113, XS165-109, XS165-117, XS165-105, XS165-154, XS165-170, XS165-155, XS165-172, XS165-169, XS165-177, XS175-45, XS175-46, XS175-59, XS175-63, XS175-64, XS175-67, XS175-68, XS175-70, XS175-71, XS175-76, XS175-110, XS175-120, XS175-126, XS175-132, XS175-133, XS175-137, XS175-143, XS175-148, XS175-149, XS175-158, XS175-159, XS175-160, XS175-173, XS175-174, XS175-175, XS175-176, XS175-178, XS175-179, XS175-180, XS175-186, XS186-3, XS185-4, XS186-5, XS185-6, XS185-24, XS185-29, XS185-43, XS185-46, XS185-59, XS185-64, XS185-65, XS185-66, XS185-67, XS185-69, XS185-70, XS185-71, XS185-72, XS185-75, XS185-78, XS185-86, XS185-87, XS185-90, 91, XS185-96, XS185-97, XS185-101, XS185-109, XS185-113, XS185-114, XS185-116, XS185-118, XS185-122, XS185-131, XS185-134, XS185-135, XS185-138, XS185-140, XS185-147, XS185-149, XS185-150, XS185-171, XS190-9, XS190-27, XS190-38, XS190-44, XS190-45, XS190-46, XS190-47, XS190-48, XS190-49, XS190-50, XS190-59, XS190-68, XS190-69, XS190-70, XS190-75, XS190-75-2, XS190-77, XS190-80, XS190-81, XS190-126, XS190-127, XS190-128, XS190-129, XS190-130, XS190-137, XS190-138, XS190-144, XS190-157, XS190-158, XS190-170, XS190-181, XS190-182, XS190-183, XS190-186, XS197-3, XS197-4, XS197-5, XS197-6, XS197-14, XS197-29, XS197-38, XS197-49, XS197-50, XS197-70, XS197-71, XS197-72, XS197-73, XS197-74, XS197-85, XS197-93, XS197-94, XS197-96, XS197-133, XS197-145, XS197-176, XS197-185, XS197-186, XS209-21, XS209-22, XS209-23, XS209-24, XS209-25, XS209-26, XS209-27, XS209-28, XS209-30, XS209-38, XS209-39, XS209-40, XS209-53, XS209-54, XS209-55, XS209-57, XS209-60, XS209-62, XS209-65, XS209-74, XS209-75, XS209-92, XS209-99, XS209-100, XS209-101, XS209-122, XS209-139, XS209-140, XS209-165, XS209-166, XS209-167, XS209-168, XS209-174, XS209-175, XS209-176, XS209-184, XS209-185, XS224-6, XS224-106, XS224-107, XS224-108, XS224-109, XS224-110, XS224-111, XS224-116, XS224-117, XS224-118, XS224-119, XS224-143, XS224-144, XS224-145, XS224-147, XS224-148, XS224-149, XS224-150, XS224-154, XS224-155, XS224-156, XS224-157, XS224-158, XS224-159, or analogs thereof.

13. An OTUB1 binder selected from

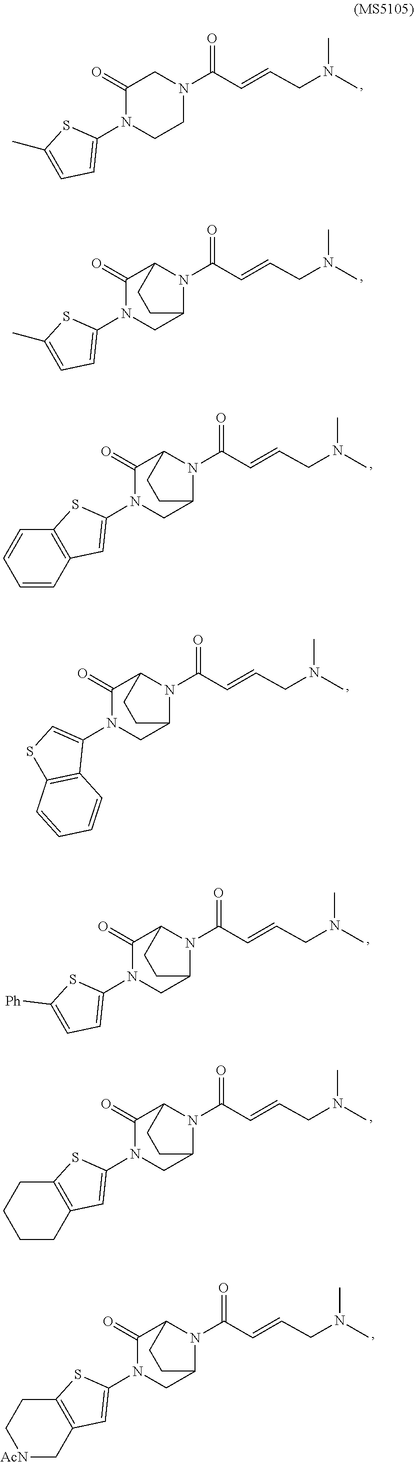

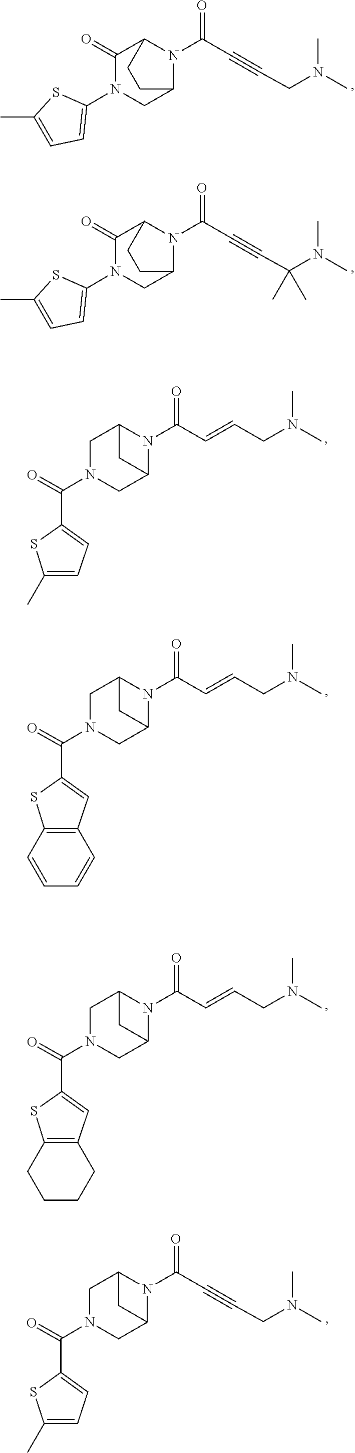

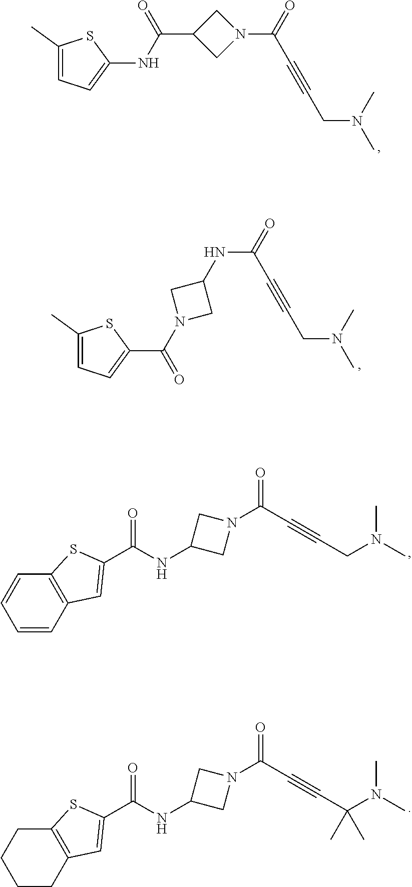

14. A bivalent compound comprising a ligand bound to a de-ubiquitination tag through a linker, the de-ubiquitination tag including an OTUB1 recruiter moiety according to Formula A-I 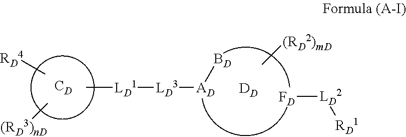
wherein
A_{D} is selected from N or CR_{D}²;
B_{D} is selected from C═O, CH₂, CH R_{D}², or C(R_{D}²)₂;
Ring C_{D} is absent, or selected from C₃-C₁₂ cycloalkyl, 3-12-membered heterocyclic, C₆-C₁₀ aryl, and 5-10 membered heteroaryl;
Ring D_{D} is a saturated or partially unsaturated 4-12 membered heterocyclic;
F_{D} is selected from N, or CR_{D}²;
L_{D}¹ is a bond, or a bivalent group selected from —O—, —NR_{D}⁵—, —C(O)—, —C(O)O—, —C(O)NR_{D}⁵-—NR_{D}⁵C(O)—, —OC(O)NR_{D}⁵—, —NR_{D}⁵C(O)O—, —NR_{D}⁶C(O)NR_{D}⁵—, —S(O)—, —S(O)₂—, —S(O)NR_{D}⁵—, —S(O)₂NR_{D}⁵—, C₁-C₆ alkylene, C₁-C₆ haloalkylene, C₁-C₆ heteroalkylene, C₂-C₆ alkenylene, and C₂-C₆ alkynylene;
L_{D}² is selected from —C(O)—, —S(O)—, —S(O)₂—, —NR_{D}⁵C(O)—, and C₁-C₆ alkylene;
L_{D}³ is a bond, or a bivalent group selected from C₁-C₆ alkylene, C₁-C₆ haloalkylene, C₁-C₆ heteroalkylene, C₂-C₆ alkenylene, and C₂-C₆ alkynylene;
R_{D}¹ is selected from C₁-C₆ haloalkyl, C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic, where each said C₂-C₆ alkenyl, C₂-C₆ alkynyl, C₂-C₆ heteroalkenyl, C₂-C₆ heteroalkynyl, partially unsaturated C₄-C₈ cycloalkyl, and partially unsaturated 4-8 membered heterocyclic were optionally substituted with hydrogen, halogen, cyano, OR_{D}⁷, NR_{D}⁷R_{D}⁸, C(O)NR_{D}⁷R_{D}⁸, C(O)OR_{D}⁷, tri(C₁-C₃alkyl) silyl, C₁-C₆ alkyl, C₁-C₆ heteroalkyl, and 3-8 membered heterocyclic;
each R_{D}² is independently selected from hydrogen, halogen, cyano, OR_{D}⁷, NR_{D}⁷R_{D}⁸, C₁-C₆ alkyl, C₁-C₆ haloalkyl, C₁-C₆ heteroalkyl, C₂-C₆ alkenyl, C₂-C₆ heteroalkenyl, C₁-C₆ alkynyl, C₂-C₆ heteroalkynyl, C₃-C₈ cycloalkyl, and 3-8 membered heterocyclic; or
two R_{D}² groups, together with the atom(s) to which they are connected, optionally form a C₃-C₁₂ cycloalkyl ring or 3-12-membered heterocyclic ring, where each said C₃-C₁₂cycloalkyl ring or 3-12-membered heterocyclic ring, together with Ring D_{D}, can optionally form fused rings or bridged rings;
each R_{D}³ is selected from hydrogen, halogen, cyano, OR_{D}⁷, NR_{D}⁷R_{D}⁸, C(O)OR_{D}⁷, C(O)NR_{D}⁷R_{D}⁸, —OC(O)NR_{D}⁷R_{D}⁸,—NR_{D}⁹C(O)NR_{D}⁷R_{D}⁸—, —S(O)NR_{D}⁷R_{D}⁸—, —S(O)₂NR_{D}⁷R_{D}⁸—, C₁-C₆ alkyl, C₁-C₆ haloalkyl, C₁-C₆ heteroalkyl, C₂-C₆ alkenyl, C₁-C₆ alkynyl, C₃-C₁₂ cycloalkyl, 3-12-membered heterocyclic, C₆-C₁₀ aryl, and 5-10 membered heteroaryl; or
two R_{D}³ groups, together with the atom(s) to which they are connected, optionally form a C₃-C₁₂ cycloalkyl ring or 3-12-membered heterocyclic ring, where each said C₃-C₁₂ cycloalkyl ring or 3-12-membered heterocyclic ring, together with Ring C_{D}, optionally form fused rings or bridged rings;
R_{D}⁴ is R_{D}^{4a} or R_{D}^{4b};
R_{D}⁴a is selected from hydrogen, OR_{D}⁷, NR_{D}⁷R_{D}⁸, C(O)R_{D}⁷, C(O)OR_{D}⁷, C(O)NR_{D}⁷R_{D}⁸, —OC(O)NR_{D}⁷R_{D}⁸, —NR_{D}⁹C(O)NR_{D}⁷R_{D}⁸—, S(O)R_{D}⁷, S(O)₂R_{D}⁷, S(O)NR_{D}⁷R_{D}⁸, S(O)₂NR_{D}⁷R_{D}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12-membered heterocyclic, C₆-C₁₀ aryl, and 5-10 membered heteroaryl;
R_{D}⁴b is optionally a bivalent group for connection to a linker, and is selected from a bond, —O—, —N—, —C(O)—, —C(O)O—, —C(O)NR_{D}⁷—, —OC(O)NR_{D}⁷—, —NR_{D}⁹C(O)NR_{D}⁷—, —S(O)—, —S(O)₂—, —S(O)NR_{D}⁷—, —S(O)₂NR_{D}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12-membered heterocyclicene, C₆-C₁₀ arylene, and 5-10 membered heteroarylene;
each R_{D}⁵ and each R_{D}⁶ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
each R_{D}⁷, each R_{D}⁸ and each R_{D}⁹ are independently selected from hydrogen, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{D}⁷ and R_{D}⁸, together with the atom(s) to which they are connected, optionally form 3-12-membered heterocyclic ring;
m_{D} is an integer of 0-8;
n_{D} is an integer of 0-8; and tautomers and pharmaceutically acceptable salts thereof.

15. The bivalent compound of claim 14, wherein the linker is a moiety according to Formula (E): 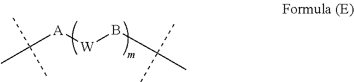
wherein
A, W and B, at each occurrence, are independently selected from null, or bivalent moiety selected from R′—R″, R′COR″, R′CO₂R′, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R¹)R″, R′N(R¹)R″, R′NR¹COR″, R′NR¹C(O)OR″, R′NR¹CON(R²)R″, R′NR¹C(S)R″, R′NR¹S(O)R″, R′NR¹S(O)₂R″, and R′NR¹S(O)₂N(R²)R″, wherein
R′ and R″ are independently selected from a bond, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R¹ and R², R′ and R¹, R′ and R², R″ and R¹, R″ and R² together with the atom to which they are connected form a 3-20 membered cycloalkyl or 4-20 membered heterocyclyl ring;
m is 0 to 15; and
and pharmaceutically acceptable salts and the tautomers thereof.

16. The bivalent compound according to claim 14, wherein the linker is a moiety according to Formula (E-1): 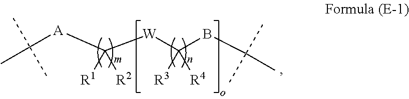
wherein
R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or
R¹ and R², R³ and R⁴ together with the atom to which they are connected form a 3-20 membered cycloalkyl or 4-20 membered heterocyclyl ring;
A, W and B, at each occurrence, are independently selected from null, or bivalent moiety selected from R′—R″, R′COR″, R′CO₂R″, R′C(O)N(R⁵)R″, R′C(S)N(R⁵)R″, R′OR″, R′OC(O)R″, R′OC(O)OR″, R′OCONR⁵R″, R′SR″, R′SOR″, R′SO₂R″, R′SO₂N(R⁵)R″, R′N(R⁵)R″, R′NR⁵COR″, R′NR⁵C(O)OR″, R′NR⁵CON(R⁶)R″, R′NR⁵C(S)R″, R′NR⁵S(O)R″, R′NR⁵S(O)₂R″, and R′NR⁵S(O)₂N(R⁶)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;
m is 0 to 15;
n, at each occurrence, is 0 to 15; and
is 0 to 15.

17. The bivalent compound according to claim 14, wherein the linker is a moiety according to Formula (E-2): 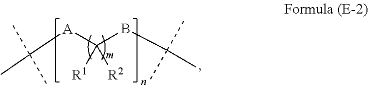
wherein
R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;
A and B, at each occurrence, are independently selected from null, or bivalent moiety selected from R′—R″, R′COR″, R′CO₂R″, R′C(O)NR³R′, R′C(S)NR³R″, R′OR″, R′OC(O)R″, R′OC(O)OR″, R′OCON(R³)R″, R′SR″, R′SOR″, R′SO₂R″, R′SO₂N(R³)R″, R′N(R³)R″, R′NR³COR″, R′NR³C(O)OR″, R′NR³CON(R⁴)R″, R′NR³C(S)R″, R′NR³S(O)R″, R′NR³S(O)₂R″, and R′NR³S(O)₂N(R⁴)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;
m, at each occurrence, is 0 to 15; and
n is 0 to 15.

18. The bivalent compound according to claim 14, wherein the linker is a moiety according to Formula (E-3): 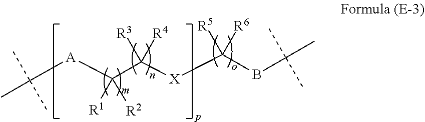
wherein
X is selected from O, NH, and NR⁷;
R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;
A and B are independently selected from null, or bivalent moiety selected from R′—R″, R′COR″, R′CO₂R″, R′C(O)N(R⁸)R″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R⁸)R″, R′N(R⁸)R″, R′NR⁸COR″, R′NR⁸C(O)OR″, R′NR⁸CON(R⁹)R″, R′NR⁸C(S)R″, R′NR⁸S(O)R″, R′NR^{B}S(O)₂R″, and R′NR⁸S(O)₂N(R⁹)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;
m, at each occurrence, is 0 to 15;
n, at each occurrence, is 0 to 15;
o is 0 to 15; and
p is 0 to 15.

19. The bivalent compound according to claim 14, wherein the linker is a moiety according to Formula (E-2-a) or (E-3-a): 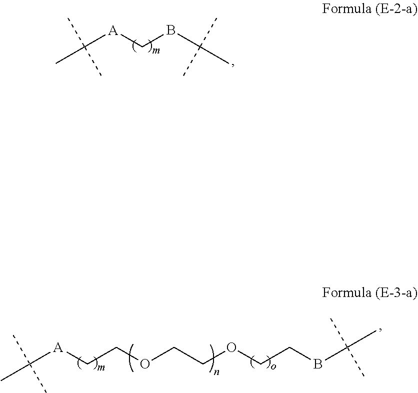
wherein
A is C═O or CH₂,
B is C═O or CH₂, and
m is 0-16,
n is 0-6,and
o is 0-6.

20. The bivalent compound according to claim 13, wherein the linker is a moiety according to Formula (E-2-b) or (E-3-b): 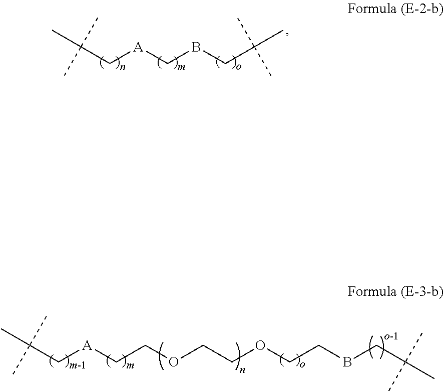
A is —C(O)N—,
B is —C(O)N—,
m is 0-16,
n is 0-6,
is 0-6,
n-1 is 0-6, and
o-1 is 0-6.

21. The bivalent compound according to any one of claims claim 14-20, wherein the OTUB1 recruiter moiety is selected from:

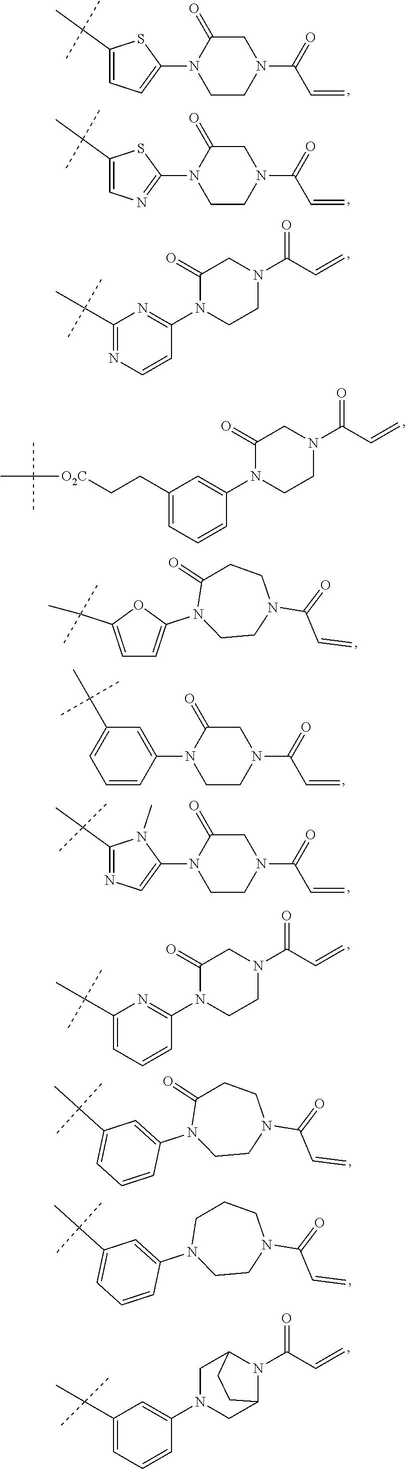

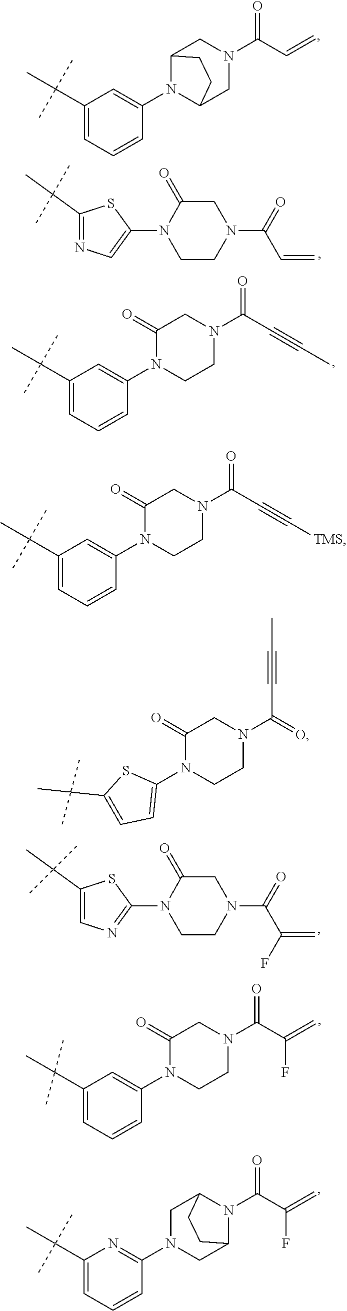

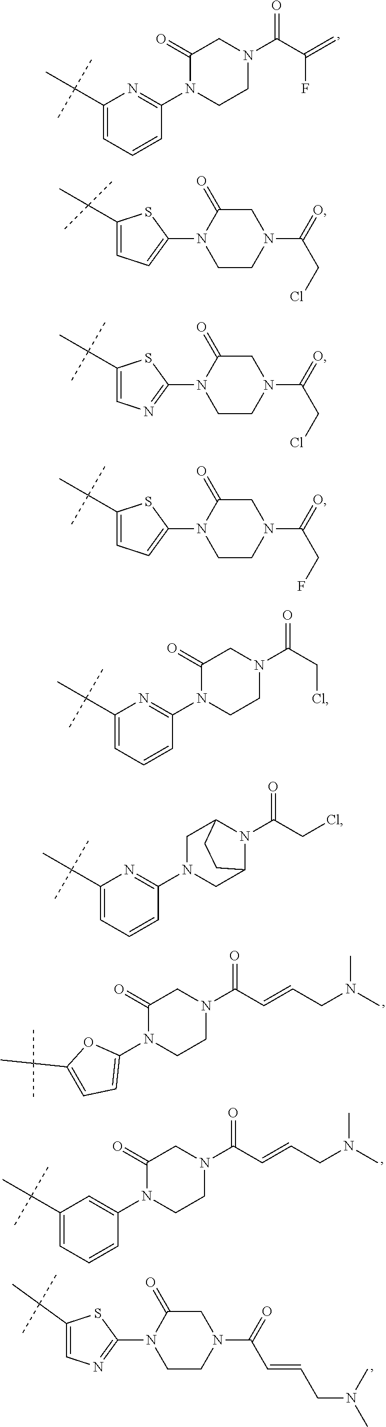

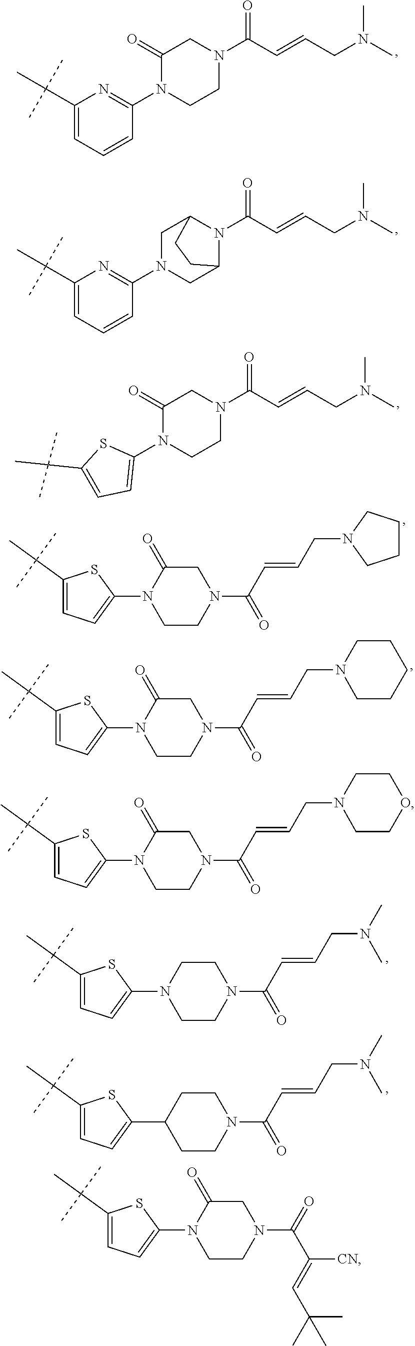

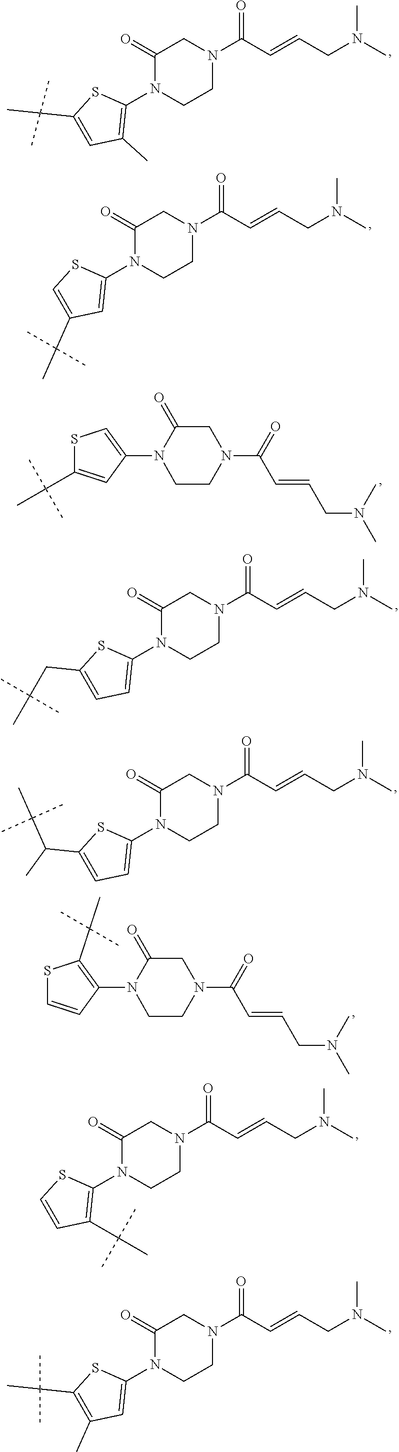

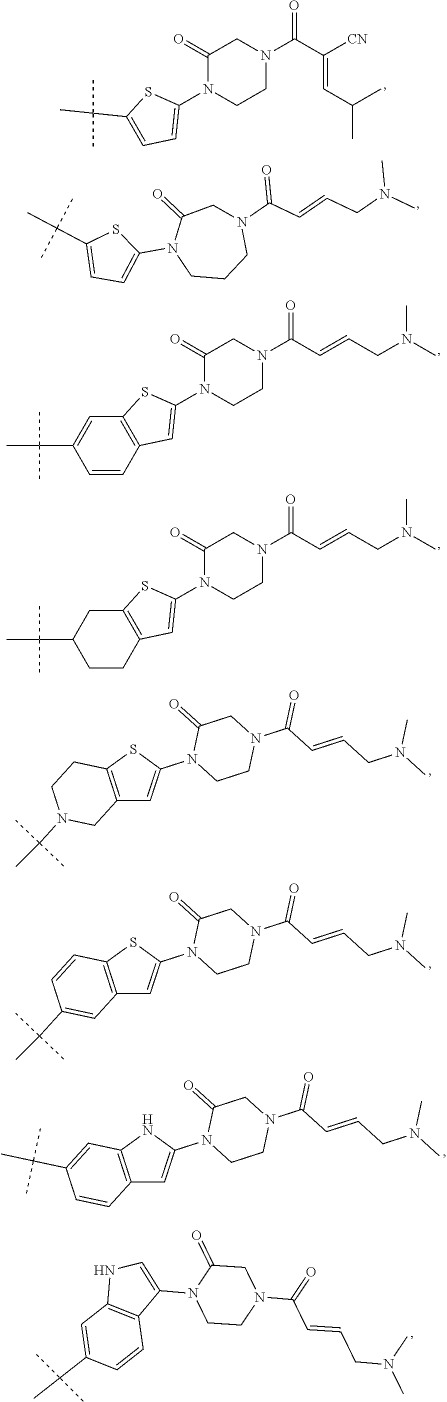

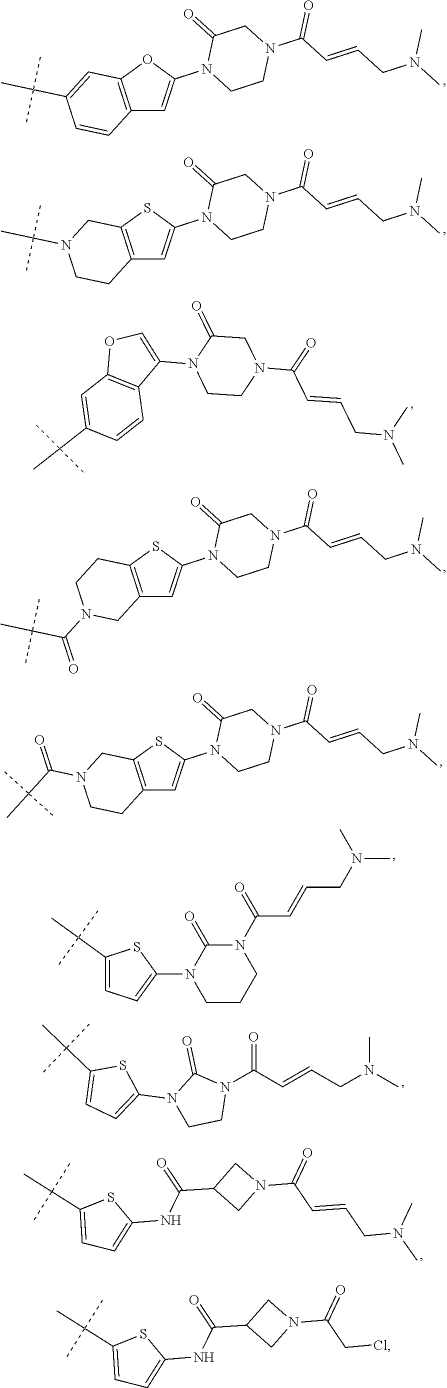

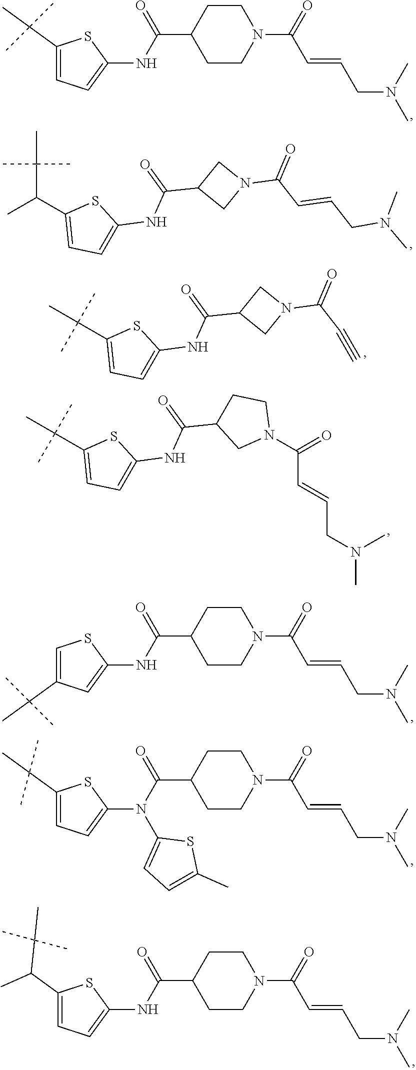

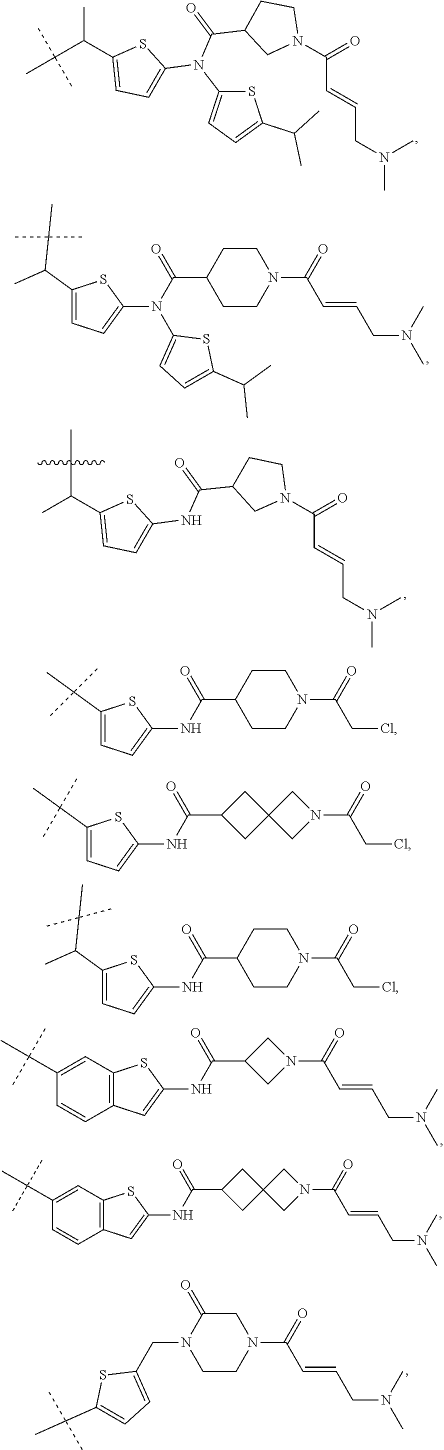

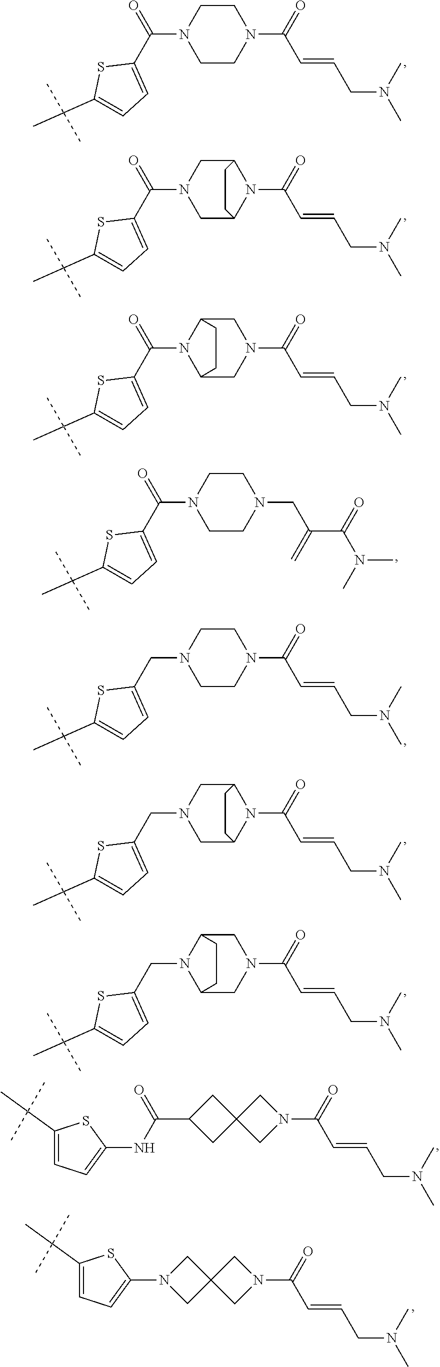

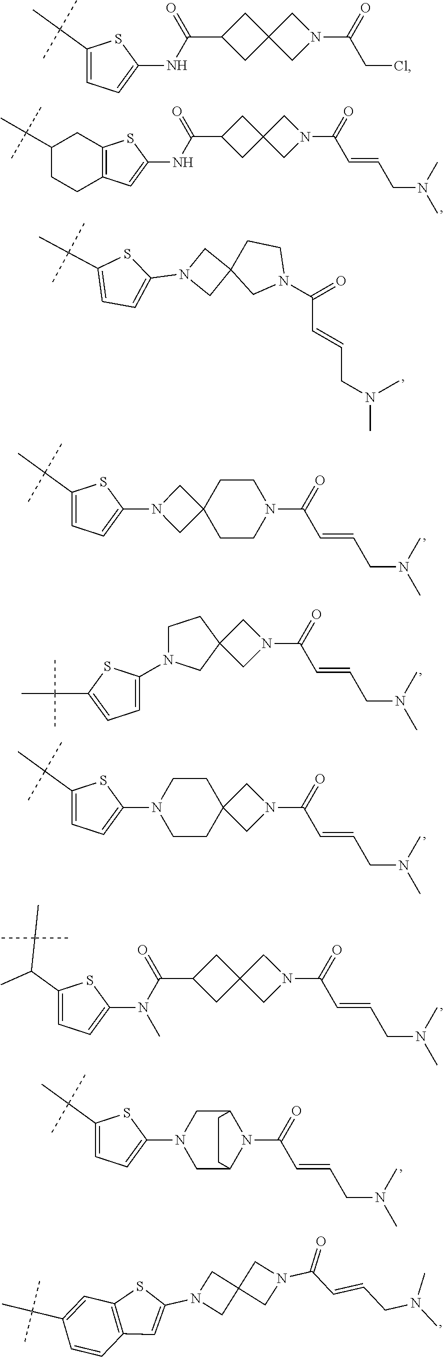

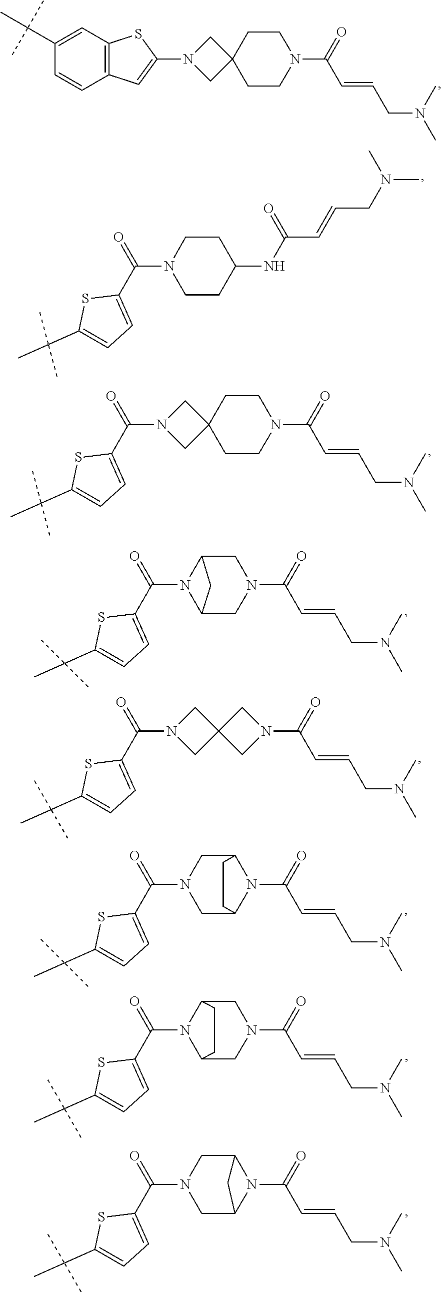

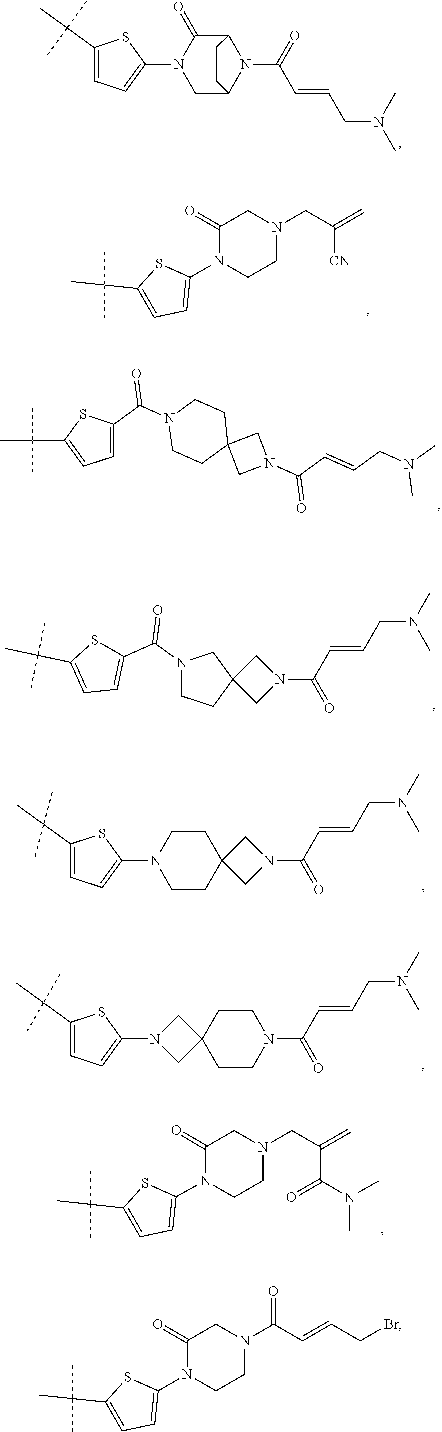

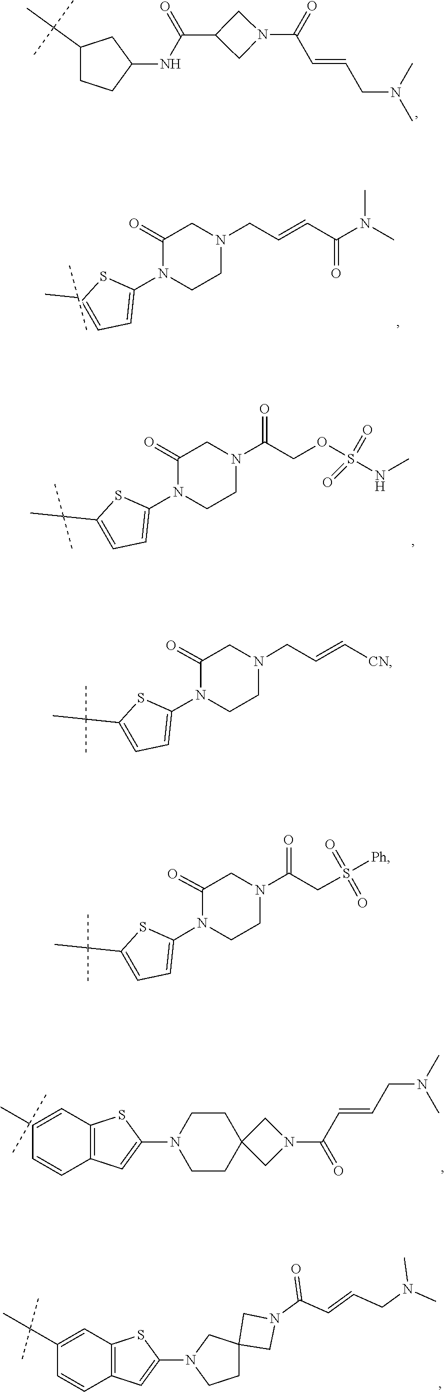

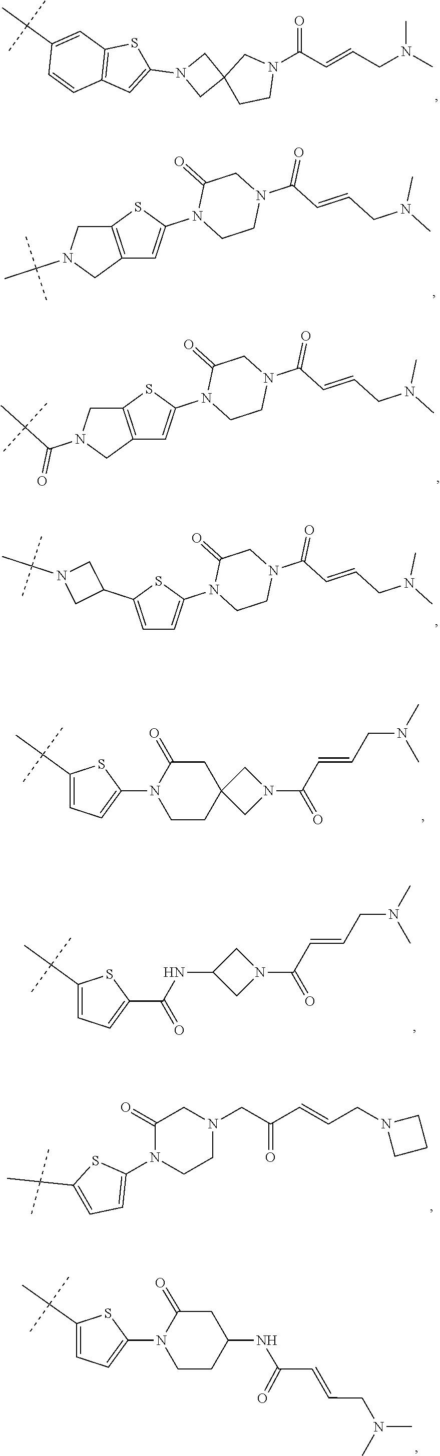

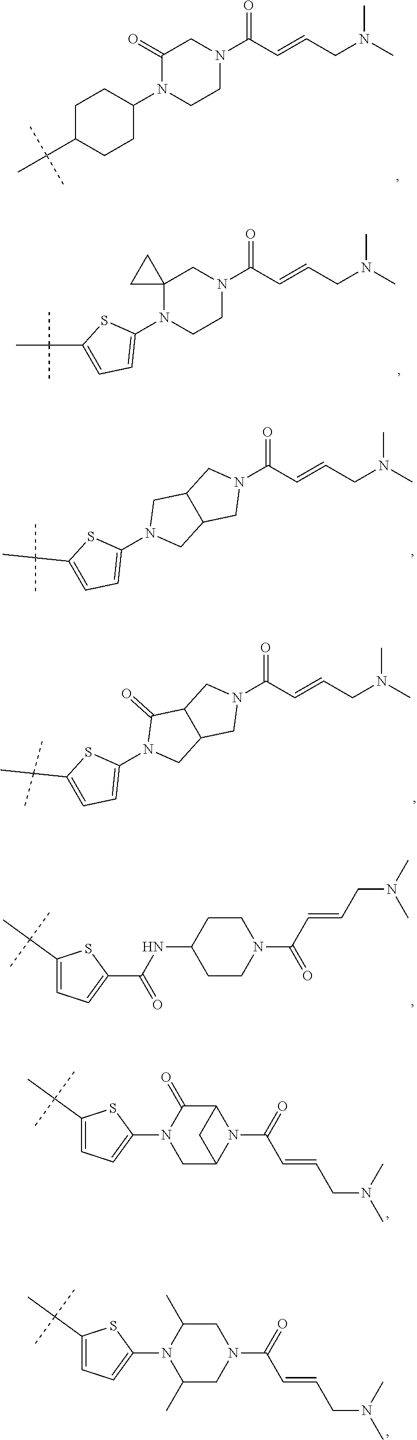

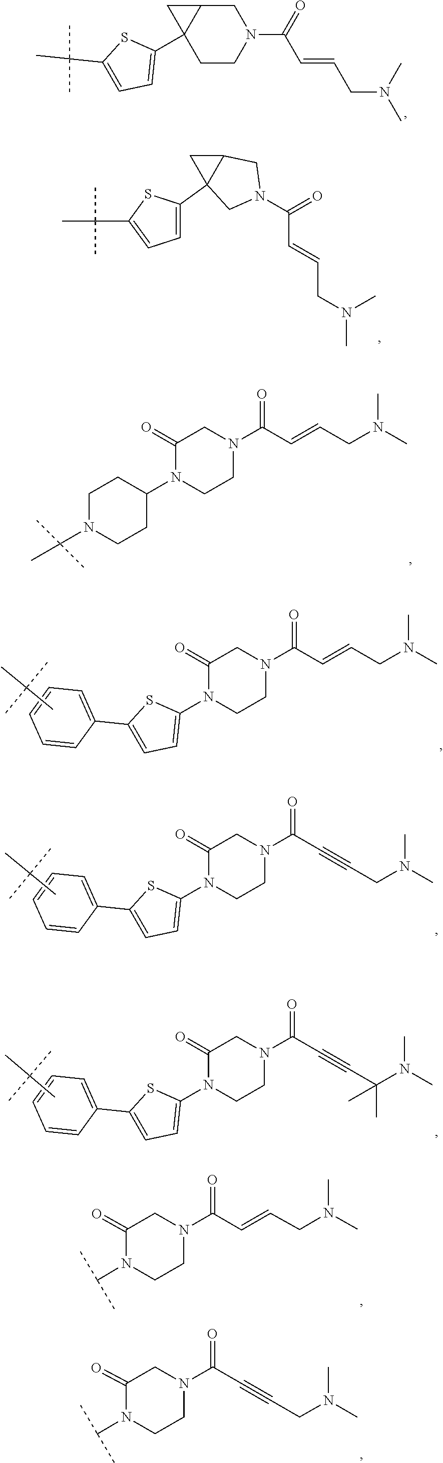

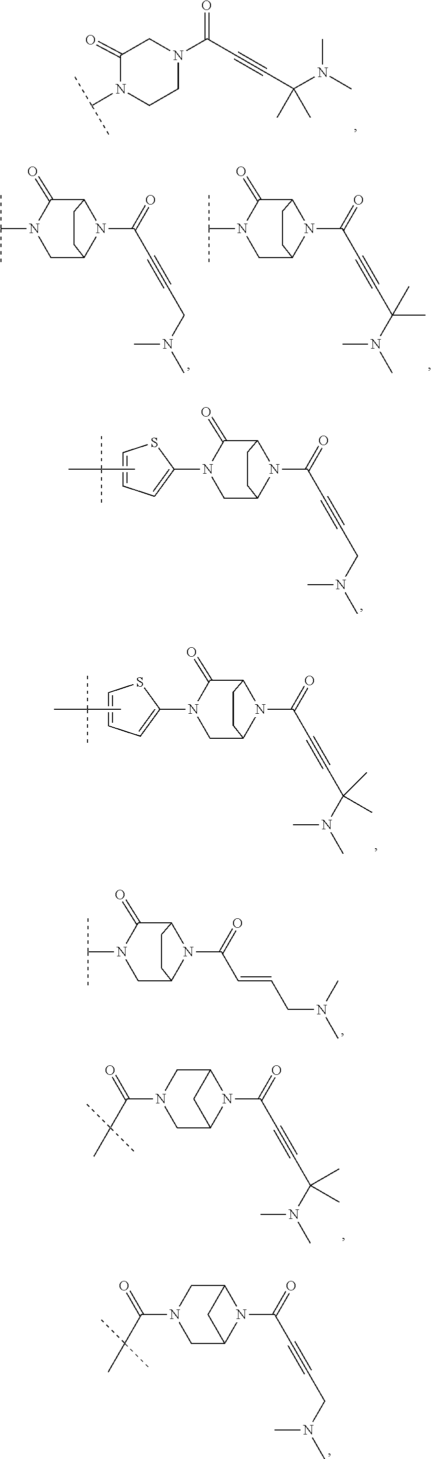

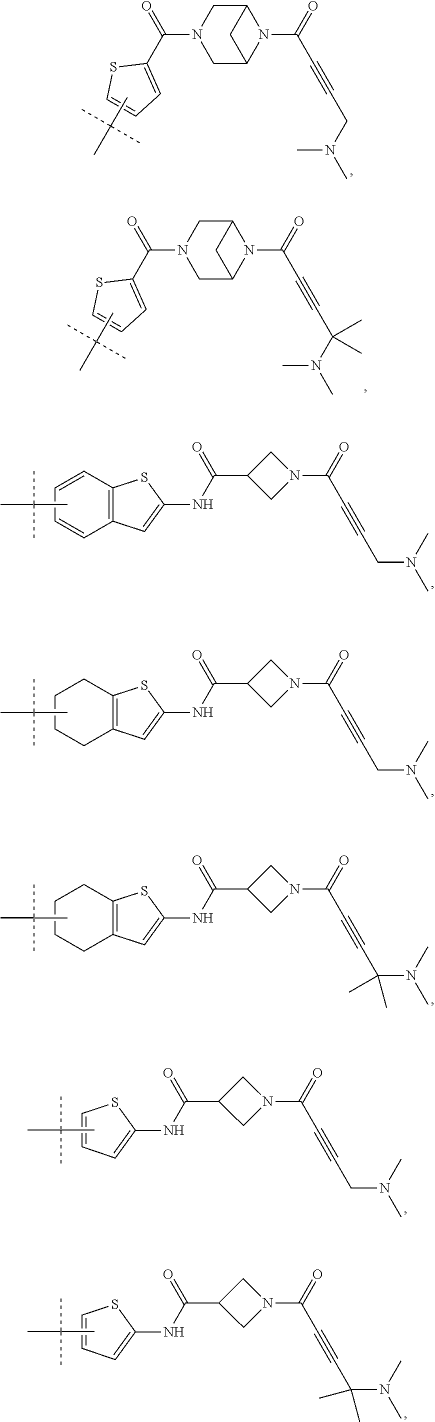

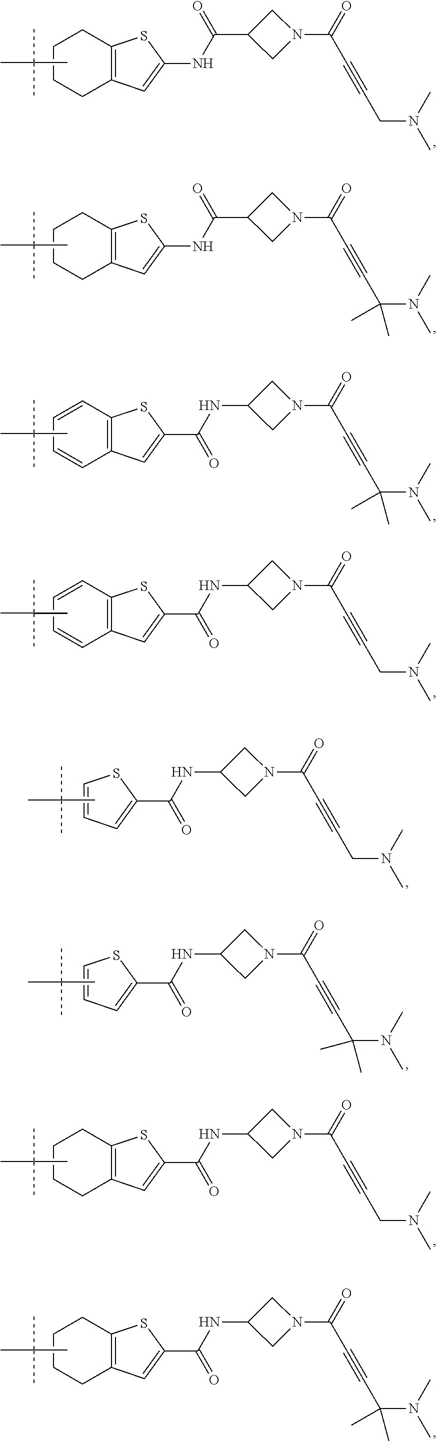

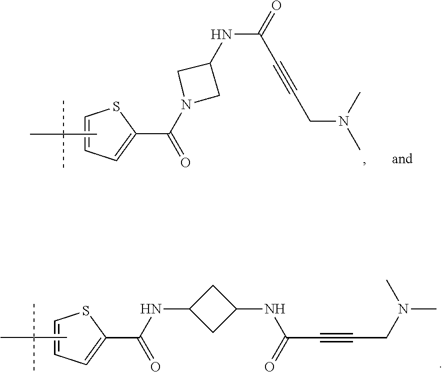

22. A bivalent compound according to any one of claims 14-21, wherein the ligand is an AMPK ligand selected from: 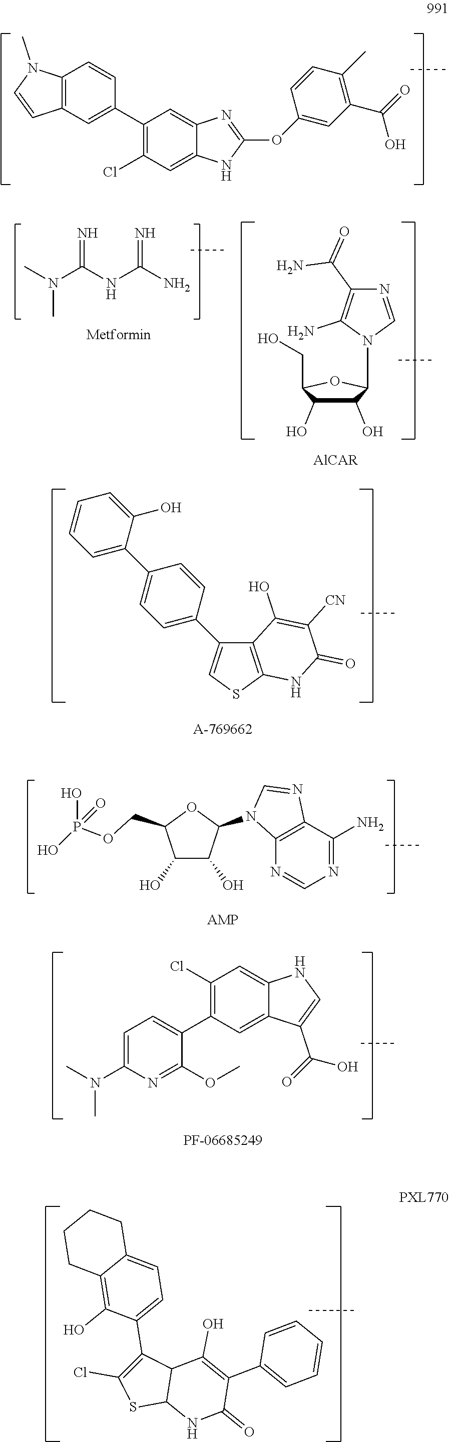 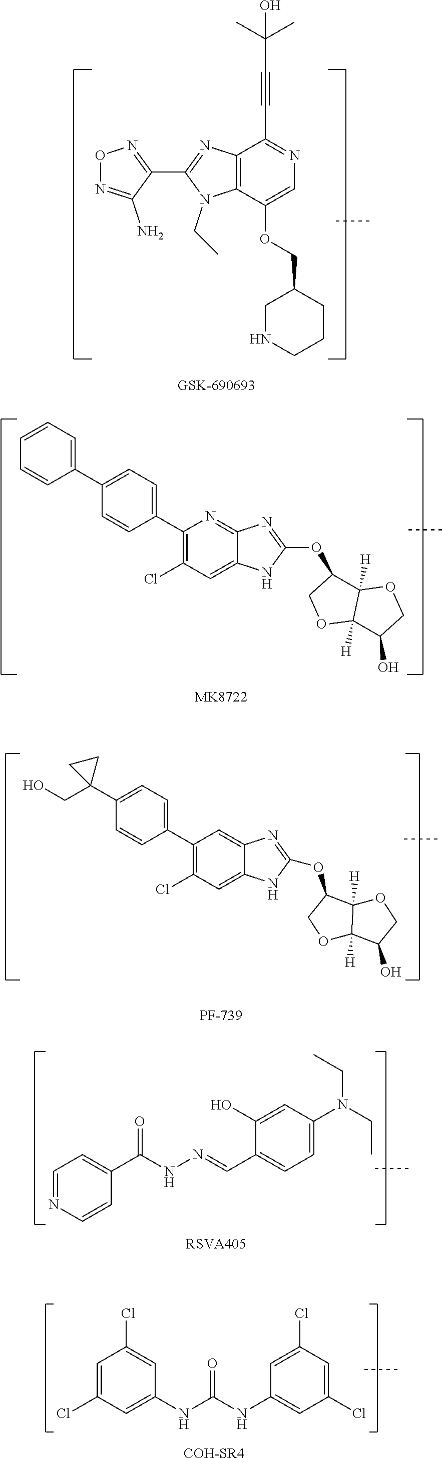 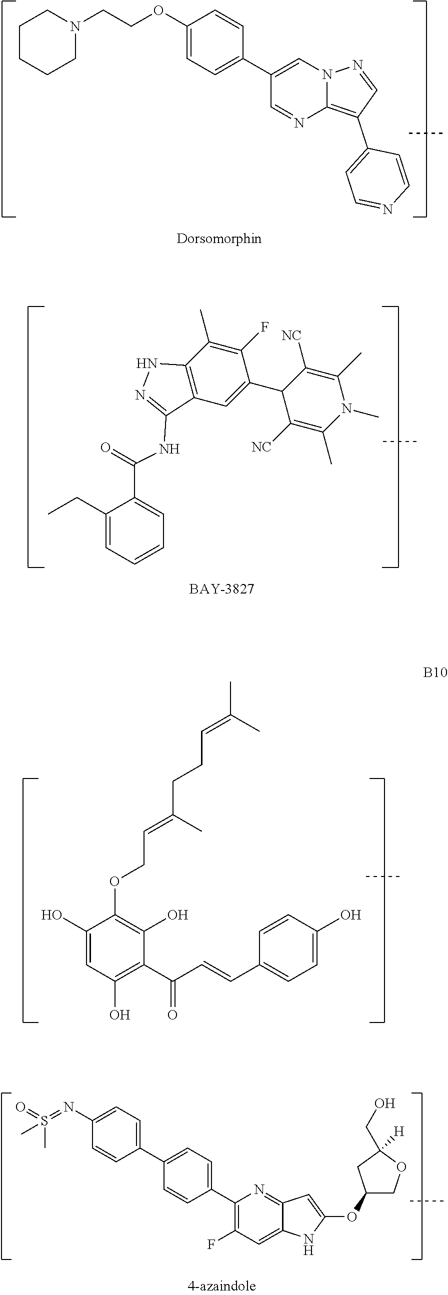
wherein [symbol] denotes the point of attachment to Linker.

23. A bivalent compound according to any one of claims 14-22, wherein the ligand is an AMPK ligand according to Formula (B-I): 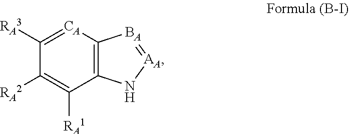
wherein
A_{A} is selected from N, CR_{A}⁴, or 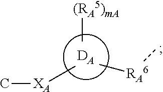
B_{A} is selected from N, CR_{A}⁴, or 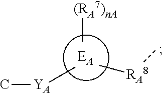 [symbol]
denotes the point of attachment to Linker in Formula (A);
C_{A} is selected from N or CR_{A}⁴;
ring D_{A} and ring E_{A} are independently selected from null, C₃-C₁₂ cycloalkyl, 3-12-membered heterocyclyl, C₆-C₁₀ aryl, and 5-10 membered heteroaryl;
X_{A} and Y_{A} are independently selected from a bond, —O—, —S—, —NR_{A}⁹—, —C(O)—, —C(O)O—, —C(O)NR_{A}⁹—, —O—C(O)NR_{A}⁹—, —NR_{A}¹⁰C(O)NR_{A}⁹—, —S(O)—, —S(O)₂—, —S(O)NR_{A}⁹—, —S(O)₂NR_{A}⁹—, C₁-C₆ alkylene, C₁-C₆ haloalkylene, C₁-C₆ heteroalkylene, C₂-C₆ alkenylene, and C₂-C₆ alkynylene;
R_{A}¹, R_{A}², each R_{A}⁴, each R_{A}⁵, and each R_{A}⁷ are independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclyl;
R_{A}³ is selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclyl, C₆-C₁₀ aryl, and 5-10 membered heteroaryl; or
R_{A}¹ and R_{A}², R_{A}² and R_{A}, RAA and R_{A}⁴, two R_{A}⁵, or two R_{A}⁷, together with the atoms to which they are attached, optionally form C₄-C₆ cycloalkyl or 5-7 membered heterocyclyl, C₆-C₁₀ aryl, and 5-10 membered heteroaryl;
R_{A}⁶ and R_{A}S are independently selected from a bond, —O—, —NR_{A}¹³—, —C(O)—, —C(O)O—, —C(O)NR_{A}¹³—, —O—C(O)NR_{A}¹³—, —NR_{A}¹⁴C(O)NR_{A}¹³—, —S(O)—, —S(O)₂—, —S(O)NR_{A}¹³—, —S(O)₂NR_{A}¹³—, C₁-C₆ alkylene, C₁-C₆ haloalkylene, C₁-C₆ heteroalkylene, C₂-C₆ alkenylene, C₂-C₆ alkynylene, C₆-C₁₀ arylene, and 5-10 membered heteroarylene;
each R_{A}⁹, each R_{A}¹⁰, each R_{A}¹¹, each R_{A}¹², each R_{A}¹³, and each R_{A}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclyl, C₆-C₁₀ aryl, and 5-10 membered heteroaryl; or
R_{A}¹¹ and R_{A}¹² together with the atoms to which they are attached, optionally form 3-12 membered heterocyclyl; and
m_{A} and n_{A} are independently selected from 0, 1, 2, 3, and 4.

24. A bivalent compound according to any one of claims 14-21, wherein the ligand is a cGAS ligand according to Formula (C-I): 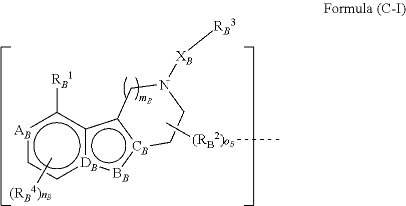
wherein [symbol]
denotes the point of attachment to Linker in Formula (A); [symbol]
connects to either R_{B}^{1b} or R_{B}^{3b}, with the proviso that when [symbol] connects to R_{B}^{1b}, R_{B}³ is R_{B}^{3a}; and when [symbol] connects to R_{B}^{3b}, R_{B}¹ is R_{B}^{1a};
A_{B} is selected from N and CR_{B}⁴;
B_{B} is selected from N, O, S, CR_{B}⁵ and NR_{B}⁵;
C_{B} is selected from N and C;
D_{B} is selected from N and C;
X_{B} is X_{B}^{a}—X_{B}^{b};
X_{B}^{a} is selected from a bond, —C(O)—, —C(O)O—, —C(O)NR_{B}⁶—, —S(O)—, —S(O)₂—, —S(O)NR_{B}⁶—, —S(O)₂NR_{B}⁶—;
X_{B}^{b} is selected from a bond, C₁-C₆ alkylene, C₁-C₆ haloalkylene, C₁-C₆ heteroalkylene, C₃-C₁₂ cycloalkylene, and 3-12 membered heterocyclylene, C₆-C₁₀ arylene, and 5-10 membered heteroarylene;
R_{B}¹ is selected from monovalent group R_{B}^{1a} and bivalent group R_{B}^{1b};
R_{B}^{1a} is selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}^{B}, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclyl, C₆-C₁₀ aryl, and 5-10 membered heteroaryl;
R_{B}^{1b} is connected to the Linker, and is selected from a bond, —O—, —NR_{B}⁷—, —C(O)O—, —C(O)NR_{B}⁷—, —S(O)₂NR_{B}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12 membered heterocyclylene, C₆-C₁₀ arylene, and 5-10 membered heteroarylene;
each R_{B}² is independently selected from H, halogen, C₁-C₆ alkyl, C₁-C₆ haloalkyl, C₁-C₆ heteroalkyl, C₃-C₁₂ cycloalkyl, or
two R_{B}², together with the atoms to which they are attached, optionally form C₃-C₁₂ cycloalkyl or 3-12 membered heterocyclyl;
R_{B}³ is selected from monovalent group R_{B}^{3a} and bivalent group R_{B}^{3b};
R_{B}^{3a} is selected from H, halogen, OR_{B}⁹, NR_{B}⁹R_{B}¹⁰, C(O)OR_{B}⁹, C(O)NR_{B}⁹R_{B}¹⁰, S(O)₂NR_{B}⁹R_{B}¹⁰, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclyl;
R_{B}^{3b} is connected to the Linker, and is selected from a bond, —O—, —NR_{B}⁹—, —C(O)O—, —C(O)NR_{B}⁹—, —S(O)₂NR_{B}⁹—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, and 3-12 membered heterocyclylene;
each R_{B}⁴ is independently selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₃ alkyl, C₁-C₃ haloalkyl, C₁-C₃ heteroalkyl, C₂-C₄ alkenyl, C₂-C₄ alkynyl, C₃-C₆ cycloalkyl, and 3-6 membered heterocyclyl, or
two R_{B}⁴, together with the atoms to which they are attached, optionally form partially unsaturated C₃-C₁₂ cycloalkyl, partially unsaturated 3-12 membered heterocyclyl, C₆-C₁₀ arylene, or 5-10 membered heteroarylene;
R_{B}⁵ is selected from H, halogen, C₁-C₃ alkyl, C₁-C₃ haloalkyl, C₁-C₃ heteroalkyl, C₃-C₆ cycloalkyl, and 3-6 membered heterocyclyl;
R_{B}⁶, each R_{B}⁷, each R_{B}⁸, each R_{B}⁹, and each R_{B}¹⁰, are independently selected from H, C₁-C₆ alkyl, C₁-C₆ haloalkyl, C₁-C₆ heteroalkyl, C₂-C₆alkenyl, C₂-C₆ alkynyl, C₃-C₁₂ cycloalkyl, and 3-12 membered heterocyclyl, or
R_{B}⁷ and R_{B}⁸, and R_{B}⁹ and R_{B}¹⁰ together with the atoms to which they are attached, optionally form 3-12 membered heterocyclyl;
m_{B} is selected from 1 and 2;
n_{B} is selected from 0, 1, 2 and 3; and
o_{B} is selected from 0, 1, 2, 3, 4, 5 and 6.

25. A bivalent compound according to any one of claims 14-21, wherein the ligand is a cGAS ligand selected from 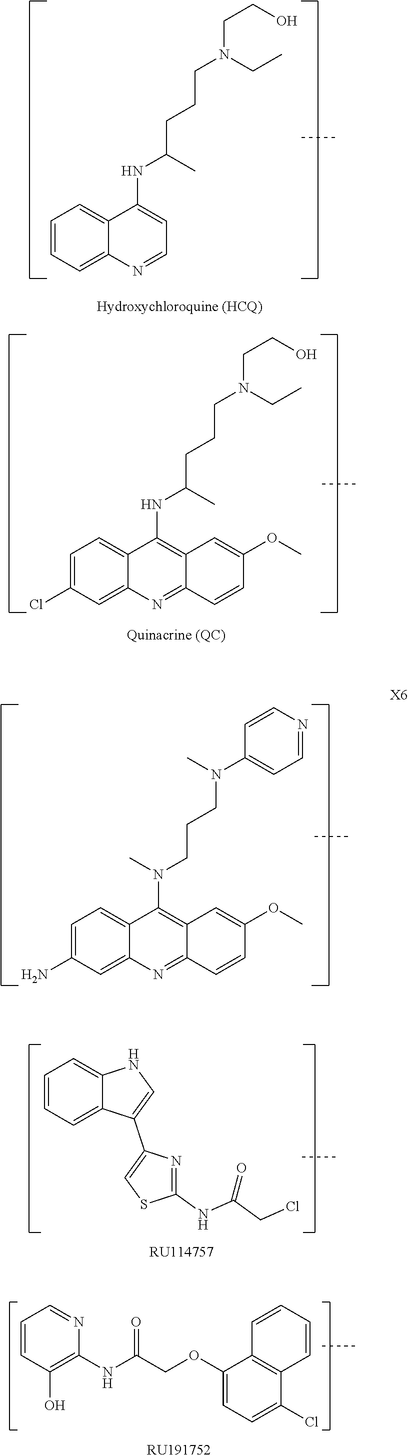 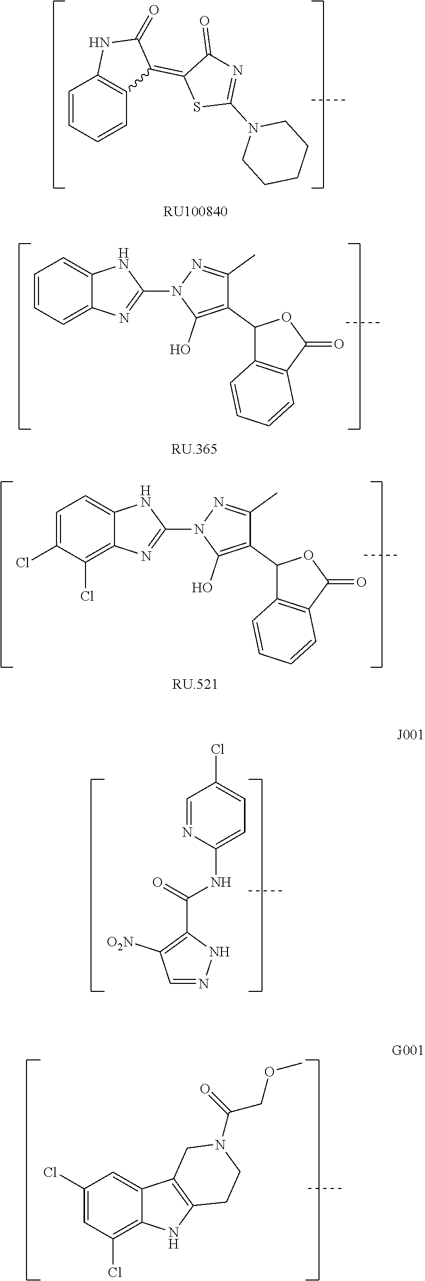 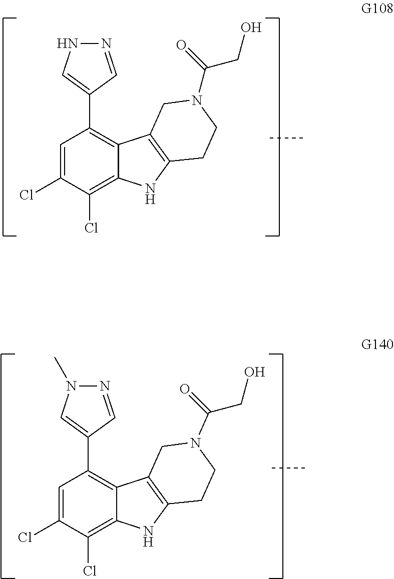 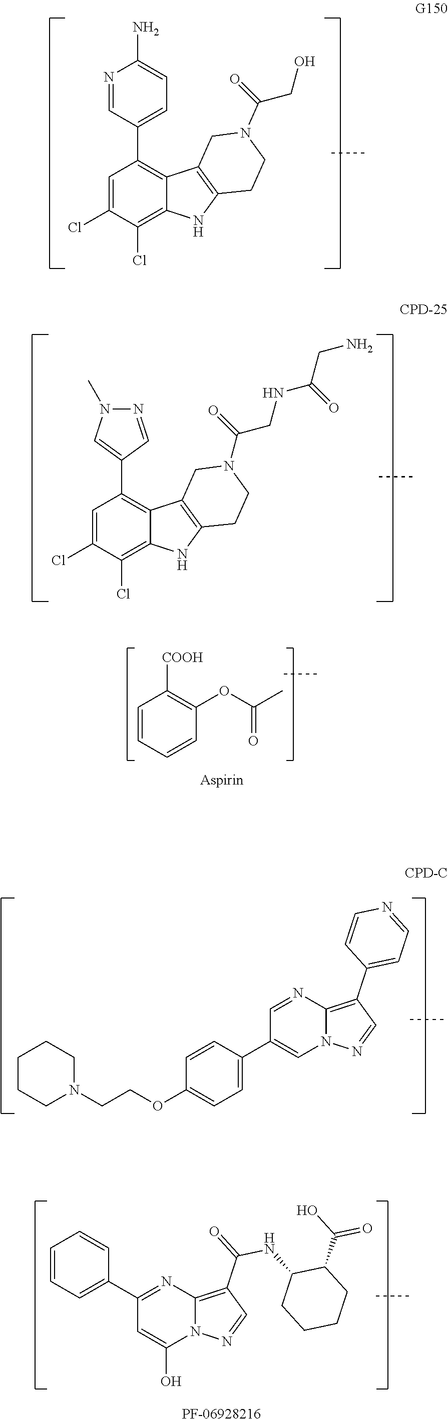
wherein, [symbol] denotes the point of attachment to Linker.

26. A bivalent compound according to any one of claims 14-21, wherein the ligand is a CFTR ligand according to Formula (D-I): 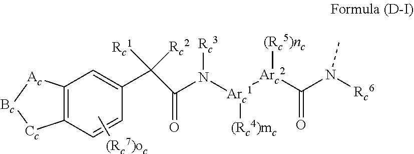
wherein [symbol]
denotes the point of attachment to the linker,
A_{C} and C_{C} are independently selected from O, S, or C(R_{C}⁸)(R_{C}⁹);
B_{C} is C(R_{C}¹⁰)(R_{C}¹¹) or NR_{C}¹²;
Ar_{C}¹ and Ar_{C}² are independently selected from null, C₆-C₁₀ aryl and 5-10 membered heteroaryl;
R_{C}¹ and R_{C}² are independently selected from H, halo, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclyl, or
R_{C}¹ and R_{C}², together with the atoms to which they are attached, optionally form C₃-C₁₂ cycloalkyl;
R_{C}³ and R_{C}⁶ are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclyl;
each R_{C}⁴, each R_{C}⁵, and each R_{C}⁷ are independently selected from H, halogen, cyano, OR_{C}¹³ NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclyl,
two R_{C}⁴, two R_{C}⁵, or two R_{C}⁷, together with the atoms to which they are attached, optionally form partially unsaturated C₃-C₁₂ cycloalkyl, partially unsaturated 3-12 membered heterocyclyl, C₆-C₁₀ aryl and 5-10 membered heteroaryl;
R_{C}⁸, R_{C}⁹, R_{C}¹⁰ and R_{C}¹¹ are independently selected from H, halogen, OR_{C}¹³ NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₃-C₁₂ cycloalkyl, and 3-12 membered heterocyclyl, or
R_{C}⁸ and R_{C}⁹, and R_{C}¹⁰ and R_{C}¹¹, together with the atoms to which they are attached, optionally form C₃-C₁₂ cycloalkyl and 3-12 membered heterocyclyl;
R_{C}¹² is selected from H, C₁-C₆ alkyl, C₁-C₆ haloalkyl, C₁-C₆ heteroalkyl, C₃-C₁₂ cycloalkyl, and 3-12 membered heterocyclyl;
R_{C}³ and R_{C}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclyl, or
R_{C}¹³ and R_{C}¹⁴ together with the atoms to which they are attached, optionally form 3-12 membered heterocyclyl;
m_{C} is 0, 1, 2, or 3;
n_{C} is 0, 1, 2, or 3; and
o_{C} is 0, 1, 2, or 3.

27. A bivalent compound according to any one of claims 14-21, wherein the ligand is a CFTR ligand selected from 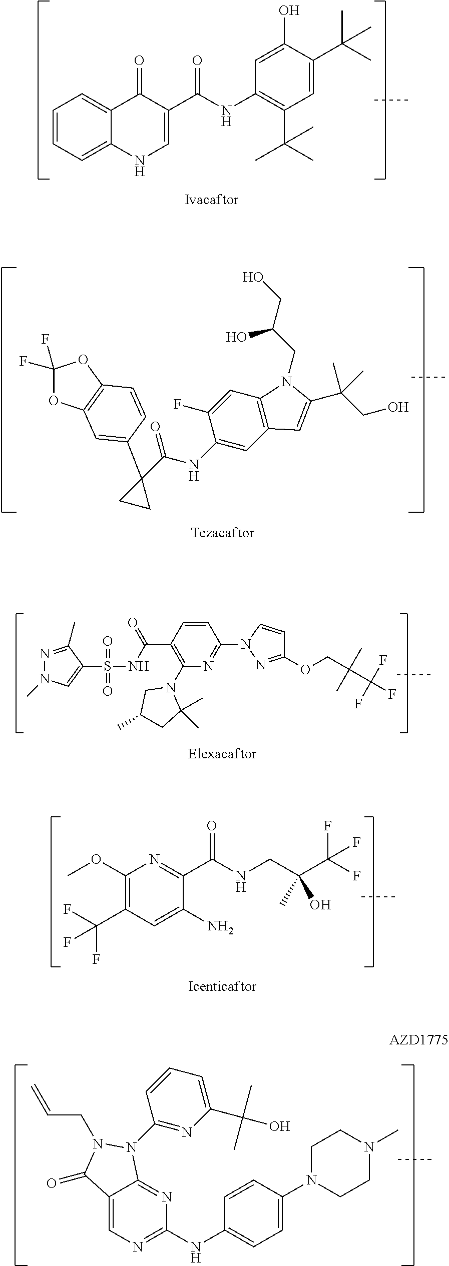 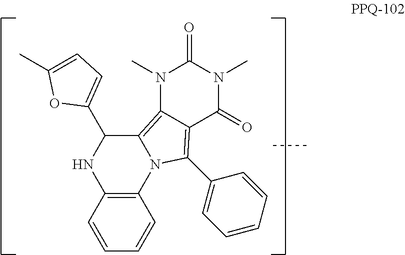
wherein, [symbol] denotes the point of attachment to Linker.

28. A bivalent compound including an AMPK ligand selected from XF137-81, XF137-82, XF137-83, XF137-84, XF137-85, XF137-86, XF137-87, XF137-88, XF137-89, XF137-90, XF137-91, XF137-92, XF137-93, XF137-94, QC179-047, QC179-048, QC137-049, QC179-050, QC179-051, QC179-052, QC179-053, QC179-054, QC137-055, and examples 367-380, or analogs thereof.

29. A bivalent compound including a cGAS ligand selected from ZD178-28, ZD178-29, ZD178-30, ZD178-31, ZD178-32, ZD178-33, ZD178-34, ZD178-35, ZD178-58-1, ZD178-58-2, ZD178-58-3, ZD178-58-4, ZD178-58-5, ZD178-58-6, ZD178-58-7, ZD178-58-8, ZD178-58-9, ZD178-58-10, ZD178-58-11, ZD178-58-12, ZD178-58-13, ZD178-58-14, ZD178-63-1, ZD178-63-2, ZD178-63-3, ZD178-63-4, ZD178-63-5, ZD178-63-6, ZD178-63-78, ZD178-63-8, ZD178-63-9, ZD178-63-10, ZD178-63-11, ZD178-63-12, ZD178-63-13, ZD178-63-14, ZD178-63-15, ZD178-69-1, ZD178-69-2, ZD178-69-3, ZD178-69-4, ZD178-69-5, ZD178-69-6, ZD178-69-7, ZD178-69-8, ZD178-69-9, ZD178-69-10, ZD178-69-11, ZD178-69-12, ZD178-69-13, ZD178-69-14, ZD178-69-15, and examples 381-388, or analogs thereof.

30. A bivalent compound including a CFTR ligand selected from QC166-130, QC166-131, QC166-132, QC166-133, QC166-134, QC166-135, QC166-136, QC166-137, QC166-138, QC166-139, QC166-140, QC166-141, QC166-142, QC166-143, QC166-167, QC166-174, QC166-175, QC166-176, QC166-177, QC166-178, QC166-179, QC166-181, QC166-182, QC166-183, QC166-184, QC166-185, QC179-104, QC179-105, QC179-106, QC179-107, QC179-108, QC179-109, QC179-110, QC179-111, QC179-112, QC179-113, QC179-137, QC179-138, QC179-139, QC179-140, QC179-141, QC179-154, QC179-155, QC179-156, QC179-157, QC179-158, QC192-153, QC192-154, QC192-155, QC192-156, QC192-157, QC192-184, QC192-178, QC192-179, QC192-180, QC192-181, QC192-182, or analogs thereof.

31. The bivalent compound (E)-N-(9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)nonyl)-3-(5-(4-(4-(dimethylamino)but-2-enoyl)-2-oxopiperazin-1-yl)thiophen-2-yl)propenamide (ZD178-29, MS7829).

32. The bivalent compound (E)-N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N11-(2-(5-(4-(4-(dimethylamino)but-2-enoyl)-2-oxopiperazin-1-yl)thiophen-2-yl)ethyl)undecanediamide (ZD178-58-8, MS8588).

33. The bivalent compound (E)-3-(6-(1-(2,2-difluorobenzo[d][1,3]dioxol-5-yl)cyclopropane-1-carboxamido)-3-methylpyridin-2-yl)-N-(7-((2-(5-(4-(4-(dimethylamino)but-2-enoyl)-2-oxopiperazin-1-yl)thiophen-2-yl)ethyl)amino)-7-oxoheptyl)benzamide (QC166-178, MS6178).

34. A pharmaceutical composition, comprising a compound according to any one of claims 1-33 and a pharmaceutically acceptable carrier.

35. A pharmaceutical composition according to claim 34, wherein the compound is present in an effective amount.

36. A pharmaceutical composition according to claim 35, wherein the composition is formulated for administration by oral, parenteral intradermal, subcutaneous, topical, rectal or transdermal delivery.

37. A method of treating an AMPK-mediated disease, comprising administering to a subject in need thereof of a bivalent compound according to any one of claims 14-23 and 28.

38. A method of treating a cGAS-mediated disease, comprising administering to a subject in need thereof of a bivalent compound according to any one of claims 14-21, 24, 25 and 29.

39. A method of treating a CFTR-mediated disease, comprising administering to a subject in need thereof of a bivalent compound according to any one of claims 14-21, 26, 27 and 30.

40. The method of any one of claims 37-39, wherein the disease is selected from cystic fibrosis, breast cancer, ovarian cancer, prostate cancer, colon cancer, pancreatic cancer, bladder cancer, liver cancer and cervical cancer.
